(19) United States
(12) Patent Application Publication
(10) Pub. No.: US 2023/0357766 A1
(43) Pub. Date: Nov. 9, 2023

(54) PRIME EDITING GUIDE RNAS, COMPOSITIONS THEREOF, AND METHODS OF USING THE SAME

(71) Applicants: The Broad Institute, Inc., Cambridge, MA (US); President and Fellows of Harvard College, Cambridge, MA (US)

(72) Inventors: David R. Liu, Cambridge, MA (US); James William Nelson, Cambridge, MA (US); Peyton Barksdale Randolph, Cambridge, MA (US); Andrew Vito Anzalone, Cambridge, MA (US); Simon Shen, Cambridge, MA (US); Kelcee Everette, Cambridge, MA (US); Peter J. Chen, Cambridge, MA (US)

(73) Assignees: The Broad Institute, Inc., Cambridge, MA (US); President and Fellows of Harvard College, Cambridge, MA (US)

(21) Appl. No.: 18/028,183

(22) PCT Filed: Sep. 24, 2021
(86) PCT No.: PCT/US2021/052097

§ 371 Date: Mar. 23, 2023

Related U.S. Application Data
(60) Provisional application No. 63/231,231, filed on Aug. 9, 2021.
(60) Provisional application No. 63/182,633, filed on Apr. 30, 2021.
(60) Provisional application No. 63/091,272, filed on Oct. 13, 2020.
(60) Provisional application No. 63/083,067, filed on Sep. 24, 2020.

(51) Int. Cl. C12N 15/113; C12N 9/22

(52) U.S. Cl. CPC C12N 15/113; C12N 9/22; C12N 2310/20; C12N 2310/3519; C12N 2310/531

(57) ABSTRACT

The disclosure provides modified pegRNAs comprising one or more appended nucleotide structural motifs which increase the editing efficiency during prime editing, increase half-life in vivo, and increase lifespan in a cell. Modifications include, but are not limited to, an aptamer (e.g., prequeosim-1 riboswitch aptamer or “evopreQi-1”) or a variant thereof, a pseudoknot (the MMLV viral genome pseudoknot or “Mpknot-1”) or a variant thereof, a tRNA (e.g., the modified tRNA used by MMLV as a primer for reverse transcription) or a variant thereof, or a G-quadruplex or a variant thereof. The disclosure further provides prime editor complexes comprising the modified pegRNAs and having improved characteristics and/or performance, including stability, improved cellular lifespan, and improved editing efficiency. The disclosure also provides methods of editing a genome using the prime editor complexes with modified pegRNAs, and to nucleotide sequences and expression vectors encoding said prime editors and modified pegRNAs, and to cells, kits, and pharmaceutical compositions comprising the improved prime editor complexes.

### RELATED APPLICATIONS

This application claims the benefit of U.S. Provisional Application Ser. No. 63/231,231, filed Aug. 9, 2021, U.S. Provisional Application Ser. No. 63/182,633, filed Apr. 30, 2021, and U.S. Provisional Application Ser. No. 63/083,067, filed Sep. 24, 2020. In addition, this application claims the benefit of U.S. Provisional Application Ser. No. 63/091,272, filed Oct. 13, 2020, the contents of each of which are incorporated herein by reference.

### GOVERNMENT SUPPORT

This invention was made with government support under grant numbers AI142756, HG009490, EB022376, and GM118062 awarded by the National Institutes of Health. The government has certain rights in the invention.

### BACKGROUND OF THE INVENTION

Prime editing (PE) is a nucleic acid editing platform that enables the targeted and programmable installation of defined changes in a nucleotide sequence at a desired locus. It involves targeting of a prime editor to a target site in the genome, wherein the prime editor comprises a nucleic acid programmable DNA binding protein (napDNAbp) fused to a polymerase (e.g., a reverse transcriptase (RT)) associated with a prime editing guide RNA (pegRNA). The pegRNA comprises a scaffold (which binds to the napDNAbp), a spacer sequence (which is complementary to the genomic site), and an extension arm at the 3′ or 5′ end of the pegRNA. The extension arm includes a DNA synthesis template which includes the sequence of the desired edit. During prime editing, once the prime editor complexed with the pegRNA localizes to the genomic site, the polymerase (e.g., reverse transcriptase) synthesizes a new strand of DNA containing a desired edit using the DNA synthesis template. The new strand of DNA then replaces the corresponding endogenous DNA strand at the genomic site, thereby installing the desired, edited nucleotide sequence into the genome at the edit site.

Despite the many advantages of prime editing over other modes of genome editing, such as the ease of programming the DNA synthesis template to specify the desired edit, it remains desirous to further enhance the characteristics and performance of prime editing, including, for example, the efficiency of installing desired edits and/or reducing indel formation.

Modifications to prime editing and/or to the components thereof which result in increased editing efficiencies and/or increased specificity would significantly advance the field of genome editing.

### SUMMARY OF THE INVENTION

The present disclosure provides next-generation pegRNAs with improved properties, including, but not limited to, increased stability, increased half-life in vivo, and/or improved binding affinity for a napDNAbp and/or a target DNA sequence. These improved properties may be achieved in various ways, including, but not limited to, appending three-dimensional RNA structures, such as stem loops, to pegRNAs to increase their stability, or modifications to reduce the binding affinity of the primer binding site (PBS) of the pegRNA extension arm to the spacer sequence of the pegRNA (e.g., through occluding the PBS with toeholds that dissociate upon napDNAbp binding, providing the 3′ extension arm in trans, or introducing chemical and/or genetic modifications to the pegRNA, as described further herein). These modified pegRNAs result in improved activity and/or efficiency of prime editing when used in conjunction with a prime editor, such as a fusion protein comprising a napDNAbp domain (e.g., a Cas9 domain) and a polymerase domain (e.g., a reverse transcriptase domain. In particular, the inventors have discovered that pegRNAs may suffer from various deficiencies, including reduced affinity to a nucleic acid programmable DNA binding protein (e.g., a Cas9 nickase), increased susceptibility to degradation compared to canonical single guide RNAs (sgRNAs) (in particular, degradation of the extension arm), and tendency toward inactivation due to unwanted duplex formation between the extension arm (specifically, the primer binding site of the extension arm) and the spacer sequence in the pegRNA, thereby competing against the binding of the pegRNA to a target DNA. Without wishing to be bound by any particular theory, these issues arise because of the presence of the extension arm that is an integral part of the pegRNA which is not present in typical sgRNAs. To overcome these deficiencies, the present inventors have discovered that pegRNAs may be modified in one or more ways to improve their overall stability and/or performance in prime editing.

First, the inventors have discovered that appending one or more RNA structural motifs to a pegRNA can protect against degradation of the pegRNA. Such RNA structural motifs can include, but are not limited to, a prequeosine1-1 riboswitch aptamer (evopreQ1) and variants thereof, a frameshifting pseudoknot from Moloney murine leukemia virus (MMLV)22, hereafter referred to as “mpknot,” and variants thereof, G-quadruplexes, hairpin structures (e.g., 15-bp hairpins), and a P4-P6 domain of the group I intron.

Second, the inventors have discovered various ways to reduce the formation of a duplex between the primer binding site (PBS) of the extension arm and the spacer sequence of the pegRNA (i.e., reducing PBS/spacer binding interactions). In one embodiment, PBS/spacer binder interaction is avoided by stabilizing the 3′ extension arm, including, but not limited to, (i) occluding the PBS with toeholds that dissociate upon napDNAbp (e.g., Cas9 nickase) binding, (ii) providing the 3′ extension arm in trans, i.e., moving the 3′ extension arm or portion thereof (e.g, PBS and/or PBS and the DNA template portions) from the pegRNA to another molecule, e.g., the nicking gRNA, and (iii) introduction of chemical and/or genetic modifications to pegRNA that favor RNA/DNA duplex formation but disfavor RNA/RNA duplex formation, thereby promoting the desired interaction between the PBS of the pegRNA and the target DNA.

Collectively, the modified pegRNAs disclosed herein resulting from the implementation of these strategies are referred to herein as “engineered” pegRNAs or “epegRNAs.”

In another aspect of the disclosure, the inventors have developed a novel computational algorithm, which may be embodied in software, for identifying one or more nucleotide linkers for coupling a prime editing guide RNA (pegRNA) to a nucleic acid moiety, such as, but not limited to, an aptamer (e.g., prequeosin₁-1 riboswitch aptamer or “evopreQ₁-1”) or a variant thereof, a pseudoknot (the MMLV viral genome pseudoknot or “Mpknot-1”) or a variant thereof, a tRNA (e.g., the modified tRNA used by MMLV as a primer for reverse transcription) or a variant thereof, or a G-quadruplex or a variant thereof, to form or result in an engineered pegRNA. The computational technique, which may be referred to herein as the pegRNA Linker Identification Tool (“pegLIT”), involves efficiently evaluating nucleic acid linker candidates to identify those which have lower propensity for base pairing to other regions of the pegRNA (e.g., regions comprising the primer binding site, spacer, DNA synthesis template, and/or gRNA core).

In addition, the present disclosure provides for nucleic acid molecules encoding and/or expressing the epegRNAs, as well as expression vectors and constructs for expressing the epegRNAs described herein, host cells comprising said nucleic acid molecules and expression vectors, and compositions for delivering and/or administering the epegRNAs in conjunction with a prime editing system described herein. In addition, the disclosure provides for isolated epegRNAs, as well as compositions comprising said epegRNAs as described herein. Still further, the disclosure provides for prime editor systems comprising (a) a prime editor (e.g., a complex or fusion protein comprising a napDNAbp (e.g., Cas9 nickase) and a reverse transcriptase or other RNA-dependent DNA polymerase) and (b) an epegRNA disclosed herein. Still further, the present disclosure provides for methods of making the epegRNAs disclosed herein, as well as methods of using the epegRNAs in methods of prime editing for introducing one or more changes into a target nucleic acid molecule, e.g., a genome, with improved efficiency as compared to a prime editor and uses a pegRNA. The specification also provides methods for efficiently editing a target nucleic acid molecule, e.g., a single nucleobase of a genome, with a prime editing system described herein (e.g., in the form of a prime editor as described herein or a vector or construct encoding same and an epegRNA described herein) or any prime editing system described previously. Still further, the specification provides therapeutic methods for treating a genetic disease and/or for altering or changing a genetic trait or condition by contacting a target nucleic acid molecule, e.g., a genome, with a prime editing system described herein or describe previously which utilizes an epegRNA described herein.

In a particular embodiment, it has been surprisingly found that by appending a nucleotide structural motif to the end of the extension arm of a pegRNA, including not but limited to, an aptamer (e.g., prequeosin₁-1 riboswitch aptamer or “evopreQ₁-1”) or a variant thereof, a pseudoknot (the MMLV viral genome pseudoknot or “Mpknot-1”) or a variant thereof, a tRNA (e.g., the modified tRNA used by MMLV as a primer for reverse transcription) or a variant thereof, or a G-quadruplex or a variant thereof, a consistent increase in editing efficiency was achieved. Thus, the present disclosure provides modified pegRNAs comprising one or more appended nucleotide structural motifs which improve the editing efficiency of prime editors when complexed therewith. In addition, the disclosure provides prime editing complexes comprising a prime editor complexed with a engineered pegRNA disclosed herein, as well as to nucleotide sequences and expression vectors encoding said modified pegRNAs, and optionally which may also encode the prime editors on the same or different vector molecules. Still further, the disclosure provides genome editing methods based on prime editing that involve the use of a prime editor associated with a modified pegRNA as disclosed herein to install a desired nucleotide sequence change at a desired site in a nucleic acid characterized by an editing efficiency that is higher than prime editing that uses a pegRNAs (i.e., those pegRNAs not modified in the manner described herein). The disclosure also provides cells and kits comprising the disclosed modified pegRNAs, or prime editing complexes comprising said modified pegRNAs. The present disclosure also provides methods of making the disclosed modified pegRNAs comprising coupling one or more structural nucleotide motifs (e.g., aptamers, G-quadruplexes, tRNAs, or pseudoknot) to the terminus of the extension arm of a pegRNA, optionally through a nucleotide linker. The disclosure further provides methods for delivering the modified pegRNAs and optionally, prime editors to target cells for conducting genome editing at a desired target site, as well as methods for treating genetic disorders using prime editing in combination with the disclosed modified pegRNAs.

The process of prime editing may introduce at least one or more of the following genetic changes into a nucleic acid (e.g., genome): transversions, transitions, deletions, and insertions. In addition, prime editing may be implemented for specific applications. For example, prime editing can be used to (a) install mutation-correcting changes to a nucleotide sequence, (b) install protein and RNA tags, (c) install immunoepitopes on proteins of interest, (d) install dimerization domains in proteins, (e) install or remove sequences that alter the activity of a biomolecule, (f) install recombinase target sites to direct specific genetic changes, and (g) mutagenize a target sequence by using an error-prone RT, as well as other purposes. And, with the modified pegRNAs described herein, these applications of prime editing may be conducted with high efficiency and/or reduced occurrence of indels.

In a first aspect, the disclosure provides a pegRNA for prime editing comprising a guide RNA and at least one nucleic acid extension arm comprising a DNA synthesis template and a primer binding site, wherein the extension arm comprises a nucleic acid moiety attached thereto selected from the group consisting of a toe-loop, hairpin, stem-loop, pseudoknot, aptamer, G-quadraplex, tRNA, riboswitch, or ribozyme. In certain embodiments, the nucleic acid moiety is attached to the 3′ end of the extension arm of the pegRNA. In other embodiments, the nucleic acid moiety is attached to the 5′ end of the extension arm of the pegRNA.

In various embodiments, the nucleic acid moiety is a Mpknot1 moiety having a nucleotide sequence selected from the group consisting of: SEQ ID NO: 195 (Mpknot1), SEQ ID NO: 196 (Mpknot1 3′ trimmed), SEQ ID NO: 197 (Mpknot1 with 5′ extra), SEQ ID NO: 198 (Mpknot1 U38A), SEQ ID NO: 199 (Mpknot1 U38A A29C), SEQ ID NO: 200 (MMLC A29C), SEQ ID NO: 201 (Mpknot1 with 5′ extra and U38A), SEQ ID NO: 202 (Mpknot1 with 5′ extra and U38A A29C), and SEQ ID NO: 203 (Mpknot1 with 5′ extra and A29C), or a nucleotide sequence having at least 80% sequence identity therewith.

In other embodiments, the nucleic acid moiety is a G-quadruplex having a nucleotide sequence selected from the group consisting of: SEQ ID NO: 204 (tns1), SEQ ID NO: 205 (stk40), SEQ ID NO: 206 (apc2), SEQ ID NO: 207 (ceacam4), SEQ ID NO: 208 (pitpnm3), SEQ ID NO: 209 (rlf), SEQ ID NO: 210 (erc1), SEQ ID NO: 211 (ube3c), SEQ ID NO: 212(taf15), SEQ ID NO: 213 (stard3), and SEQ ID NO: 214 (g2), or a nucleotide sequence having at least 80% sequence identity therewith.

In still other embodiments, the nucleic acid moiety that modifies a pegRNA is an evopreq1 aptamer having a nucleotide sequence selected from the group consisting of: SEQ ID NO: 215 (evopreq1), SEQ ID NO: 216 (evopreq1motif1), SEQ ID NO: 217 (evopreq1motif2), SEQ ID NO: 218 (evopreq1motif3), SEQ ID NO: 219 (shorter preq1-1), SEQ ID NO: 220 (preq1-1 G5C (mut1)), and SEQ ID NO: 221 (preq1-1 G15C (mut2)), or a nucleotide sequence having at least 80% sequence identity therewith.

In still other embodiments, the nucleic acid moiety is a the tRNA moiety having a nucleotide sequence of SEQ ID NO: 222, or a nucleotide sequence having at least 80% sequence identity therewith.

In yet other embodiments, the nucleic acid moiety has a nucleotide sequence of SEQ ID NO: 223 (xrn1), or a nucleotide sequence having at least 80% sequence identity therewith.

In other embodiments, the nucleic acid moiety has a nucleotide sequence of SEQ ID NO: 224 (grp1 intron P4P6), or a nucleotide sequence having at least 80% sequence identity therewith.

Any of the nucleic acid moieties described herein can be attached to the pegRNA, e.g., to the 3′ end of the pegRNA, by a linker, e.g., a nucleotide linker. The linker can have a nucleotide sequence selected from the group consisting of SEQ ID NOs: 225-236. The linker can be of any suitable sequence. Optionally, the linker sequence can be determined empirically for each pegRNA.

The linker can be of any suitable length. In certain embodiments, the linker is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides in length.

In a preferred embodiment, the linker is at least 8 nucleotides in length.

In various embodiments, the extension arm of the pegRNA is positioned at the 3′ or 5′ end of the guide RNA, or at an intramolecular position in the guide RNA, and wherein the nucleic acid extension arm is DNA or RNA.

In various embodiments, the pegRNA is capable of binding to a napDNAbp and directing the napDNAbp to a target DNA sequence. The target DNA sequence can comprise a target strand and a complementary non-target strand. The guide RNA can hybridize to the target strand to form an RNA-DNA hybrid and an R-loop.

In various embodiments, the length of the extension arm can vary, and depends upon the length of the DNA synthesis template. In certain embodiments, the nucleic acid extension arm is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides.

The DNA synthesis template can also vary depending on the desired edit and can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length.

In various embodiments, the desired edit is a single nucleotide substitution, or a single nucleotide deletion, or insertion. The desired edits can also be of any length capable of being installed by prime editing, and can include deletions, insertions, or inversions.

The primer binding site can also vary in length and can be, for example, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length.

In another aspect, the disclosure provides pegRNAs for prime editing comprising (i) a guide RNA comprising a spacer and (ii) at least one nucleic acid extension arm comprising a DNA synthesis template, a primer binding site, a toehold motif, and an additional nucleic acid moiety, wherein the toehold motif occludes interaction of the primer binding site and the spacer when the PEgRNA is not bound by a prime editor, but does not occlude interaction of the primer binding site and a protospacer sequence on a target DNA molecule when the PEgRNA is bound by a prime editor. In some embodiments, the toehold motif and the additional nucleic acid moiety are attached to the 3′ end of the extension arm. In some embodiments, the toehold motif is attached to the 3′ end of the extension arm, and the additional nucleic acid moiety is attached to the 3′ end of the toehold motif. In some embodiments, the toehold motif is attached to the PEgRNA by a linker.

In another aspect, the disclosure provides pairs of PEgRNAs for prime editing comprising (i) a first PEgRNA comprising a guide RNA, wherein the guide RNA comprises a spacer; and (ii) a second PEgRNA comprising a second strand nicking guide RNA, wherein the second strand nicking guide RNA comprises at least one nucleic acid extension arm comprising a DNA synthesis template and a primer binding site. In some embodiments, the first PEgRNA and the second PEgRNA are each capable of binding to a nucleic acid programmable DNA binding protein (napDNAbp) of a prime editor and directing the napDNAbp to a target DNA sequence.

In another aspect, the disclosure provides a PEgRNA comprising (i) a guide RNA comprising a spacer and (ii) at least one nucleic acid extension arm comprising a DNA synthesis template and a primer binding site, wherein the primer binding site comprises one or more modified nucleotides, wherein the one or more modified nucleotides result in a greater reduction in binding affinity of the primer binding site to the spacer than of the primer binding site to a protospacer sequence on a target DNA molecule. In some embodiments, the one or more modified nucleotides comprise genetic mutations. In some embodiments, the one or more modified nucleotides comprise chemically-modified nucleotides.

In another aspect, the disclosure provides a complex for prime editing comprising:
(a) a fusion protein comprising a nucleic acid programmable DNA binding protein (napDNAbp) and a domain comprising an RNA-dependent DNA polymerase activity; and
(b) any pegRNA described above which comprises a nucleic acid moiety appended to the end of the extension arm.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (Cas(Φ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (Cas(Φ), and Argonaute and optionally has a nickase activity such that one DNA strand is cut preferentially to the other DNA strand. In various embodiments, the domain comprising an RNA-dependent DNA polymerase activity is a reverse transcriptase comprising any one of the amino acid sequences of SEQ ID NOs: 32, 34, 36, 102-128, and 132.

The domain comprising an RNA-dependent DNA polymerase activity, in some embodiments, is a reverse transcriptase comprising an amino acid sequence having at least 80%, 85%, 90%, 95%, 98%, or 99% sequence identity with the amino acid sequence of any one of SEQ ID NOs: 32, 34, 36, 102-128, and 132. In other embodiments, the domain comprising an RNA-dependent DNA polymerase activity is a naturally-occurring reverse transcriptase from a retrovirus or a retrotransposon.

In another aspect, the disclosure provides a nucleic acid molecule encoding a modified pegRNA described above and provided in this disclosure.

In yet another aspect, the disclosure provides an expression vector comprising the above nucleic acid molecule. The nucleic acid molecule can be under the control of a promoter. The promoter can be a polIII promoter. The promoter can also be a U6, U6v4, U6v7, or U6v9 promoter or a fragment thereof, including a promoter having a nucleotide sequence of any of SEQ ID NOs: 3915-3918.

In yet another aspect, the disclosure provides cells (e.g., transformed cell lines) that comprise the modified pegRNA described above. The cells can also comprise the prime editing complexes described above (e.g., wherein the cell comprises both a modified pegRNA and a prime editor). The cells can also comprise any of the nucleic acid molecules described above, which express the modified pegRNA, and optionally which express the prime editors. In addition, the cells can comprise any of the expression vectors described above, which express the modified pegRNA, and optionally which express the prime editors.

In another aspect, the disclosure provides a pharmaceutical composition comprising: (i) a modified pegRNA described above, or a prime editing complex described above, a nucleic acid molecule described above, or an expression vector described above, or any of the cells described above, and (ii) a pharmaceutically acceptable excipient.

In yet another aspect, the disclosure provides a kit comprising: (i) a modified pegRNA described above, or a prime editing complex described above, a nucleic acid molecule described above, or an expression vector described above, or any of the cells described above, and (ii) a set of instructions for conducting prime editing.

In another aspect, the disclosure provides systems comprising (i) any of the pegRNAs or epegRNAs disclosed herein, and (ii) at least one prime editor comprising a napDNAbp and a DNA polymerase.

In another aspect, the disclosure provides a method of prime editing comprising contacting a target DNA sequence with a modified pegRNA described above and a prime editor comprising a napDNAbp and a domain having an RNA-dependent DNA polymerase activity, wherein the editing efficiency is increased as compared to the same method using a pegRNA not comprising the modification. In certain embodiments, the editing efficiency is increased by at least 1.5 fold. In other embodiments, the editing efficiency is increased by at least 2.0 fold. In still other embodiments, the editing efficiency is increased by at least 3.0 fold. In yet other embodiments, the editing efficiency is increased by at least 4, 5, 6, 7, 8, 9, or 10 fold.

In another aspect, the present disclosure uses a prime editor (e.g., PE1, PE2, or PE3) in combination with a guide RNA (pegRNA) to carry out prime editing to directly install or correct mutations in the CDKL5 gene which cause CDKL5 deficiency disorder. In various embodiments, the disclosure provides a complex comprising a prime editor (e.g., PE1, PE2, or PE3) and a pegRNA that is capable of directly installing or correcting more than one mutation in the CDKL5 gene in multiple subjects.

In the methods of prime editing disclosed herein, the napDNAbp can have a nickase activity. The napDNAbp can be a Cas9 protein or variant thereof. The napDNAbp can also be a nuclease active Cas9, a nuclease inactive Cas9 (dCas9), or a Cas9 nickase (nCas9). The napDNAbp can also be a Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (Cas(Φ), and Argonaute and optionally have a nickase activity.

In the methods of prime editing, the RNA-dependent DNA polymerase activity can be a reverse transcriptase comprising any one of the amino acid sequences of SEQ ID NOs: 32, 34, 36, 102-128, and 132. In other embodiments, the RNA-dependent DNA polymerase activity can be a reverse transcriptase comprising an amino acid sequence having at least 80%, 85%, 90%, 95%, 98%, or 99% sequence identity with the amino acid sequence of any one of SEQ ID NOs: 32, 34, 36, 102-128, and 132.

This Specification further refers to and incorporates by reference the following applications relating to prime editing, namely, U.S. Provisional Application No. 62/820,813, filed Mar. 19, 2019 (Attorney Docket No. B1195.70074US00), U.S. Provisional Application No. 62/858,958 (Attorney Docket No. B1195.70074US01), filed Jun. 7, 2019, U.S. Provisional Application No. 62/889,996 (Attorney Docket No. B1195.70074US02), filed Aug. 21, 2019, U.S. Provisional Application No. 62/922,654, filed Aug. 21, 2019 (Attorney Docket No. B1195.70083US00), U.S. Provisional Application No. 62/913,553 (Attorney Docket No. B1195.70074US03), filed Oct. 10, 2019, U.S. Provisional Application No. 62/973,558 (Attorney Docket No. B1195.70083US01), filed Oct. 10, 2019, U.S. Provisional Application No. 62/931,195 (Attorney Docket No. B1195.70074US04), filed Nov. 5, 2019, U.S. Provisional Application No. 62/944,231 (Attorney Docket No. B1195.70074US05), filed Dec. 5, 2019, U.S. Provisional Application No. 62/974,537 (Attorney Docket No. B1195.70083US02), filed Dec. 5, 2019, U.S. Provisional Application No. 62/991,069 (Attorney Docket No. B1195.70074US06), filed Mar. 17, 2020, and U.S. Provisional Application No. (63/100,548) (Attorney Docket No. B1195.70083US03), filed Mar. 17, 2020. In addition, this U.S. Provisional Application refers to and incorporates by reference International PCT Application Nos.: PCT/US20/23721; PCT/US20/23730; PCT/US20/23713; PCT/US20/23712; PCT/US20/23727; PCT/US20/23724; PCT/US20/23725; PCT/US20/23728; PCT/US20/23732; PCT/US20/23723; PCT/US20/23553; and PCT/US20/23583, each filed on Mar. 19, 2020.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A provides a schematic of an exemplary process for introducing a nucleotide change, insertion, and/or deletion into a DNA molecule (e.g., a genome) using a fusion protein comprising a reverse transcriptase fused to a Cas9 protein (i.e., a prime editor) in complex with a pegRNA (i.e., a prime editor complex). In this embodiment, the guide RNA is extended at the 3′ end to include a DNA synthesis template sequence. The schematic shows how a polymerase (e.g., a reverse transcriptase (RT)) fused to a Cas9 nickase, in a complex with a pegRNA binds the DNA target site and nicks the PAM-containing DNA strand adjacent to the target nucleotide. The RT uses the nicked DNA as a primer for DNA synthesis from the gRNA, which is used as a template for the synthesis of a new DNA strand that encodes the desired edit (e.g., mutation, insertion, and/or deletion). The editing process shown may be referred to as “prime editing.”

FIG. 1B provides the same representation as in FIG. 1A, except that the prime editor complex is represented more generally as [napDNAbp]-[P]:pegRNA or [P]-[napDNAbp]:pegRNA, wherein “P” refers to any polymerase (e.g., a reverse transcriptase), “napDNAbp” refers to a nucleic acid programmable DNA binding protein (e.g., SpCas9), and “pegRNA” refers to a prime editing guide RNA, and “]-[” refers to an optional linker. As described elsewhere, e.g., FIGS. 3A-3G, the pegRNA comprises an 5′ extension arm comprising a primer binding site and a DNA synthesis template. Although not shown, it is contemplated that the extension arm of the pegRNA (i.e., which comprises a primer binding site and a DNA synthesis template) can be DNA or RNA. The particular polymerase contemplated in this configuration will depend upon the nature of the DNA synthesis template. For instance, if the DNA synthesis template is RNA, then the polymerase case be an RNA-dependent DNA polymerase (e.g., reverse transcriptase). If the DNA synthesis template is DNA, then the polymerase can be a DNA-dependent DNA polymerase.

FIG. 1C provides a schematic of an exemplary process for introducing a single nucleotide change, insertion, and/or deletion into a DNA molecule (e.g., a genome) using a fusion protein comprising a reverse transcriptase fused to a Cas9 protein in complex with a pegRNA. In this embodiment, the guide RNA is extended at the 5′ end to include a reverse transcriptase template sequence. The schematic shows how a reverse transcriptase (RT) fused to a Cas9 nickase, in a complex with a pegRNA binds the DNA target site and nicks the PAM-containing DNA strand adjacent to the target nucleotide. The RT uses the nicked DNA as a primer for DNA synthesis from the gRNA, which is used as a template for the synthesis of a new DNA strand that encodes the desired edit. The editing process shown may be referred to as “prime editing.”

FIG. 1D provides the same representation as in FIG. 1C, except that the prime editor complex is represented more generally as [napDNAbp]-[P]:pegRNA or [P]-[napDNAbp]:pegRNA, wherein “P” refers to any polymerase (e.g., a reverse transcriptase), “napDNAbp” refers to a nucleic acid programmable DNA binding protein (e.g., SpCas9), and “pegRNA” refers to a prime editing guide RNA, and “]-[” refers to an optional linker. As described elsewhere, e.g., FIGS. 3A-3G, the pegRNA comprises an 3′ extension arm comprising a primer binding site and a DNA synthesis template. Although not shown, it is contemplated that the extension arm of the pegRNA (i.e., which comprises a primer binding site and a DNA synthesis template) can be DNA or RNA. The particular polymerase contemplated in this configuration will depend upon the nature of the DNA synthesis template. For instance, if the DNA synthesis template is RNA, then the polymerase case be an RNA-dependent DNA polymerase (e.g., reverse transcriptase). If the DNA synthesis template is DNA, then the polymerase can be a DNA-dependent DNA polymerase. In various embodiments, the pegRNA can be engineered or synthesized to incorporate a DNA-based DNA synthesis template.

FIG. 1E is a schematic depicting an exemplary process of how the synthesized single strand of DNA (which comprises the desired nucleotide change) becomes resolved such that the desired nucleotide change is incorporated into the DNA. As shown, following synthesis of the edited strand (or “mutagenic strand”), equilibration with the endogenous strand, flap cleavage of the endogenous strand, and ligation leads to incorporation of the DNA edit after resolution of the mismatched DNA duplex through the action of endogenous DNA repair and/or replication processes.

FIG. 1F is a schematic showing that “opposite strand nicking” can be incorporated into the resolution method of FIG. 1E to help drive the formation of the desired product versus the reversion product. In opposite strand nicking, a second Cas9/gRNA complex is used to introduce a second nick on the opposite strand from the initial nicked strand. This induces the endogenous cellular DNA repair and/or replication processes to preferentially replace the unedited strand (i.e., the strand containing the second nick site).

FIG. 1G provides another schematic of an exemplary process for introducing a single nucleotide change, and/or insertion, and/or deletion into a DNA molecule (e.g., a genome) of a target locus using a nucleic acid programmable DNA binding protein (napDNAbp) complexed with a pegRNA. This process may be referred to as an embodiment of prime editing. The pegRNA comprises an extension at the 3′ or 5′ end of the guide RNA, or at an intramolecular location in the guide RNA. In step (a), the napDNAbp/gRNA complex contacts the DNA molecule, and the gRNA guides the napDNAbp to bind to the target locus. In step (b), a nick in one of the strands of DNA (the R-loop strand, or the PAM-containing strand, or the non-target DNA strand, or the protospacer strand) of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence. In step (c), the 3′ end DNA strand interacts with the extended portion of the guide RNA in order to prime reverse transcription. In certain embodiments, the 3′ ended DNA strand hybridizes to a specific RT priming sequence on the extended portion of the guide RNA. In step (d), a reverse transcriptase is introduced which synthesizes a single strand of DNA from the 3′ end of the primed site towards the 3′ end of the guide RNA. This forms a single-strand DNA flap comprising the desired nucleotide change (e.g., the single base change, insertion, or deletion, or a combination thereof). In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the complementary sequence on the other strand. The process can also be driven towards product formation with second strand nicking, as exemplified in FIG. 1F. This process may introduce at least one or more of the following genetic changes: transversions, transitions, deletions, and insertions.

FIG. 1H is a schematic depicting the types of genetic changes that are possible with the prime editing processes described herein. The types of nucleotide changes achievable by prime editing include deletions (including short and long deletions), single-nucleotide changes (including transitions and transversions), inversions, and insertions (including short and long deletions).

FIG. 1I is a schematic depicting temporal second strand nicking exemplified by PE3b (PE3b=PE2 prime editor+pegRNA+second strand nicking guide RNA). Temporal second strand nicking is a variant of second strand nicking in order to facilitate the formation of the desired edited product. The “temporal” term refers to the fact that the second-strand nick to the unedited strand occurs only after the desired edit is installed in the edited strand. This avoids concurrent nicks on both strands to lead to double-stranded DNA breaks.

FIG. 1J depicts a variation of prime editing contemplated herein that replaces the napDNAbp (e.g., SpCas9 nickase) with any programmable nuclease domain, such as zinc finger nucleases (ZFN) or transcription activator-like effector nucleases (TALEN). As such, it is contemplated that suitable nucleases do not necessarily need to be “programmed” by a nucleic acid targeting molecule (such as a guide RNA), but rather, may be programmed by defining the specificity of a DNA-binding domain, such as and in particular, a nuclease. Just as in prime editing with napDNAbp moieties, it is preferable that such alternative programmable nucleases be modified such that only one strand of a target DNA is cut. In other words, the programmable nucleases should function as nickases, preferably. Once a programmable nuclease is selected (e.g., a ZFN or a TALEN), then additional functionalities may be engineered into the system to allow it to operate in accordance with a prime editing-like mechanism. For example, the programmable nucleases may be modified by coupling (e.g., via a chemical linker) an RNA or DNA extension arm thereto, wherein the extension arm comprises a primer binding site (PBS) and a DNA synthesis template. The programmable nuclease may also be coupled (e.g., via a chemical or amino acid linker) to a polymerase, the nature of which will depend upon whether the extension arm is DNA or RNA. In the case of an RNA extension arm, the polymerase can be an RNA-dependent DNA polymerase (e.g., reverse transcriptase). In the case of a DNA extension arm, the polymerase can be a DNA-dependent DNA polymerase (e.g., a prokaryotic polymerase, including Pol I, Pol II, or Pol III, or a eukaryotic polymerase, including Pol a, Pol b, Pol g, Pol d, Pol e, or Pol z). The system may also include other functionalities added as fusions to the programmable nucleases, or added in trans to facilitate the reaction as a whole (e.g., (a) a helicase to unwind the DNA at the cut site to make the cut strand with the 3′ end available as a primer, (b) a flap endonuclease (e.g., FEN1) to help remove the endogenous strand on the cut strand to drive the reaction towards replacement of the endogenous strand with the synthesized strand, or (c) a nCas9:gRNA complex to create a second site nick on the opposite strand, which may help drive the integration of the synthesize repair through favored cellular repair of the non-edited strand). In an analogous manner to prime editing with a napDNAbp, such a complex with an otherwise programmable nuclease could be used to synthesize and then install a newly synthesized replacement strand of DNA carrying an edit of interest permanently into a target site of DNA.

FIG. 1K depicts, in one embodiment, the anatomical features of a target DNA that may be edited by prime editing. The target DNA comprises a “non-target strand” and a “target strand.” The target-strand is the strand that becomes annealed to the spacer of a pegRNA of a prime editor complex that recognizes the PAM site (in this case, NGG, which is recognized by the canonical SpCas9-based prime editors) The target strand may also be referred to as the “non-PAM strand” or the “non-edit strand.” By contrast, the non-target strand (i.e., the strand containing the protospacer and the PAM sequence of NGG) may be referred to as the “PAM-strand” or the “edit strand.” In various embodiments, the nick site of the PE complex will be in the protospacer on the PAM-strand (e.g., with the SpCas9-based PE). The location of the nick will be characteristic of the particular Cas9 that forms the PE. For example, with an SpCas9-based PE, the nick site in the phosphodiester bond between bases three (“−3” position relative to the position 1 of the PAM sequence) and four (“−4” position relative to position 1 of the PAM sequence). The nick site in the protospacer forms a free 3′ hydroxyl group, which as seen in the following figures, complexes with the primer binding site of the extension arm of the pegRNA and provides the substrate to begin polymerization of a single strand of DNA code for by the DNA synthesis template of the extension arm of the pegRNA. This polymerization reaction is catalyzed by the polymerase (e.g., reverse transcriptase) of the prime editor in the 5′ to 3′ direction. Polymerization terminates before reaching the gRNA core (e.g., by inclusion of a polymerization termination signal, or secondary structure, which functions to terminate the polymerization activity of PE), producing a single strand DNA flap that is extended from the original 3′ hydroxyl group of the nicked PAM strand. The DNA synthesis template codes for a single strand DNA that is homologous to the endogenous 5′-ended single strand of DNA that immediately follows the nick site on the PAM strand and incorporates the desired nucleotide change (e.g., single base substitution, insertion, deletion, inversion). The position of the desired edit can be in any position following downstream of the nick site on the PAM strand, which can include position +1, +2, +3, +4 (the start of the PAM site), +5 (position 2 of the PAM site), +6 (position 3 of the PAM site), +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, +60, +61, +62, +63, +64, +65, +66, +67, +68, +69, +70, +71, +72, +73, +74, +75, +76, +77, +78, +79, +80, +81, +82, +83, +84, +85, +86, +87, +88, +89, +90, +91, +92, +93, +94, +95, +96, +97, +98, +99, +100, +101, +102, +103, +104, +105, +106, +107, +108, +109, +110, +111, +112, +113, +114, +115, +116, +117, +118, +119, +120, +121, +122, +123, +124, +125, +126, +127, +128, +129, +130, +131, +132, +133, +134, +135, +136, +137, +138, +139, +140, +141, +142, +143, +144, +145, +146, +147, +148, +149, or +150, or more (relative to the downstream position of the nick site). Once the 3′end single stranded DNA (containing the edit of interest) replaces the endogenous 5′ end single stranded DNA, the DNA repair and replication processes will result in permanent installation of the edit on the PAM strand, and then correction of the mismatch on the non-PAM strand that will exist at the target site. In this way, the edit will extend to both strands of DNA on the target DNA site. It will be appreciated that reference to “edited strand” and “non-edited” strand only intends to delineate the strands of DNA involved in the PE mechanism. The “edited strand” is the strand that first becomes edited by replacement of the 5′ ended single strand DNA immediately downstream of the nick site with the synthesized 3′ ended single stranded DNA containing the desired edit. The “non-edited” strand is the strand pair with the edited strand, but which itself also becomes edited through repair and/or replication to be complementary to the edited strand, and in particular, the edit of interest.

FIG. 1L depicts the mechanism of prime editing showing the anatomical features of the target DNA, prime editor complex, and the interaction between the pegRNA and the target DNA. First, a prime editor comprising a fusion protein having a polymerase (e.g., reverse transcriptase) and a napDNAbp (e.g., SpCas9 nickase, e.g., a SpCas9 having a deactivating mutation in an HNH nuclease domain (e.g., H840A) or a deactivating mutation in a RuvC nuclease domain (D10A)) is complexed with a pegRNA and DNA having a target DNA to be edited. The pegRNA comprises a spacer, gRNA core (aka gRNA scaffold or gRNA backbone) (which binds to the napDNAbp), and an extension arm. The extension arm can be at the 3′ end, the 5′ end, or somewhere within the pegRNA molecule. As shown, the extension arm is at the 3′ end of the pegRNA. The extension arm comprises in the 3′ to 5′ direction a primer binding site and a DNA synthesis template (comprising both an edit of interest and regions of homology (i.e., homology arms) that are homologous with the 5′ ended single stranded DNA immediately following the nick site on the PAM strand. As shown, once the nick is introduced thereby producing a free 3′ hydroxyl group immediately upstream of the nick site, the region immediately upstream of the nick site on the PAM strand anneals to a complementary sequence at the 3′ end of the extension arm referred to as the “primer binding site,” creating a short double-stranded region with an available 3′ hydroxyl end, which forms a substrate for the polymerase of the prime editor complex. The polymerase (e.g., reverse transcriptase) then polymerase as strand of DNA from the 3′ hydroxyl end to the end of the extension arm. The sequence of the single stranded DNA is coded for by the DNA synthesis template, which is the portion of the extension arm (i.e., excluding the primer binding site) that is “read” by the polymerase to synthesize new DNA. This polymerization effectively extends the sequence of the original 3′ hydroxyl end of the initial nick site. The DNA synthesis template encodes a single strand of DNA that comprises not only the desired edit, but also regions that are homologous to the endogenous single strand of DNA immediately downstream of the nick site on the PAM strand. Next, the encoded 3′ ended single strand of DNA (i.e., the 3′ single strand DNA flap) displaces the corresponding homologous endogenous 5′-ended single strand of DNA immediately downstream of the nick site on the PAM strand, forming a DNA intermediate having a 5′-ended single strand DNA flap, which is removed by the cell (e.g., by a flap endonuclease). The 3′-ended single strand DNA flap, which anneals to the complement of the endogenous 5′-ended single strand DNA flap, is ligated to the endogenous strand after the 5′ DNA flap is removed. The desired edit in the 3′ ended single strand DNA flap, now annealed and ligate, forms a mismatch with the complement strand, which undergoes DNA repair and/or a round of replication, thereby permanently installing the desired edit on both strands.

FIG. 2 shows three Cas complexes (SpCas9, SaCas9, and LbCas12a) that can be used in the herein described prime editors and their PAM, gRNA, and DNA cleavage features. The figure shows designs for complexes involving SpCas9, SaCas9, and LbCas12a.

FIGS. 3A-3F show designs for engineered 5′ prime editor gRNA (FIG. 3A), 3′ prime editor gRNA (FIG. 3B), and an intramolecular extension (FIG. 3C). The pegRNA may also be referred to herein as pegRNA or “prime editing guide RNA.” FIG. 3D and FIG. 3E provide additional embodiments of 3′ and 5′ prime editor gRNAs (pegRNAs), respectively. FIG. 3F illustrates the interaction between a 3′ end prime editor guide RNA with a target DNA sequence. The embodiments of FIGS. 3A-3C depict exemplary arrangements of the reverse transcription template sequence (i.e., or more broadly referred to as a DNA synthesis template, as indicated, since the RT is only one type of polymerase that may be used in the context of prime editors), the primer binding site, and an optional linker sequence in the extended portions of the 3′, 5′, and intramolecular versions, as well as the general arrangements of the spacer and core regions. The disclosed prime editing process is not limited to these configurations of pegRNAs. The embodiment of FIG. 3D provides the structure of an exemplary pegRNA contemplated herein. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a optional homology arm, a DNA synthesis template, and a primer binding site (PBS). In addition, the pegRNA may comprise an optional 3′ end modifier region (e1) and an optional 5′ end modifier region (e2). Still further, the pegRNA may comprise a transcriptional termination signal at the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The pegRNA could comprise, in certain embodiments, secondary RNA structure, such as, but not limited to, hairpins, stem/loops, toe loops, RNA-binding protein recruitment domains (e.g., the MS2 aptamer which recruits and binds to the MS2cp protein). For instance, such secondary structures could be position within the spacer, the gRNA core, or the extension arm, and in particular, within the e1 and/or e2 modifier regions. In addition to secondary RNA structures, the pegRNAs could comprise (e.g., within the e1 and/or e2 modifier regions) a chemical linker or a poly(N) linker or tail, where “N” can be any nucleobase. In some embodiments (e.g., as shown in FIG. 72(c)), the chemical linker may function to prevent reverse transcription of the sgRNA scaffold or core. In addition, in certain embodiments (e.g., see FIG. 72(c)), the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the pegRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the pegRNA (e.g., in the e1 or e2 region, or elsewhere and an MS2cp protein fused to the DNA polymerase, thereby co-localizing the DNA polymerase to the pegRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

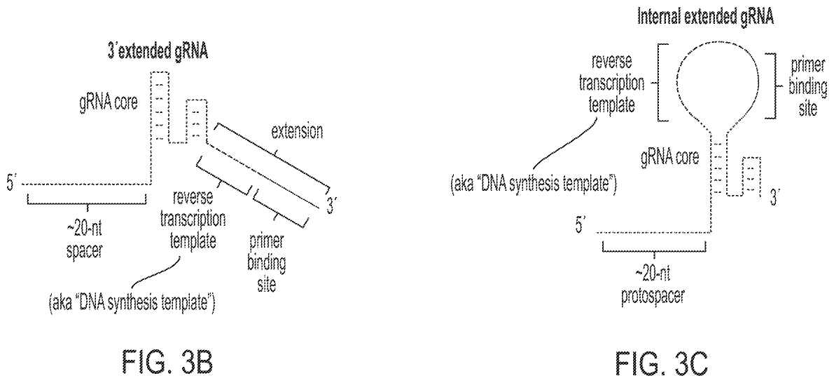

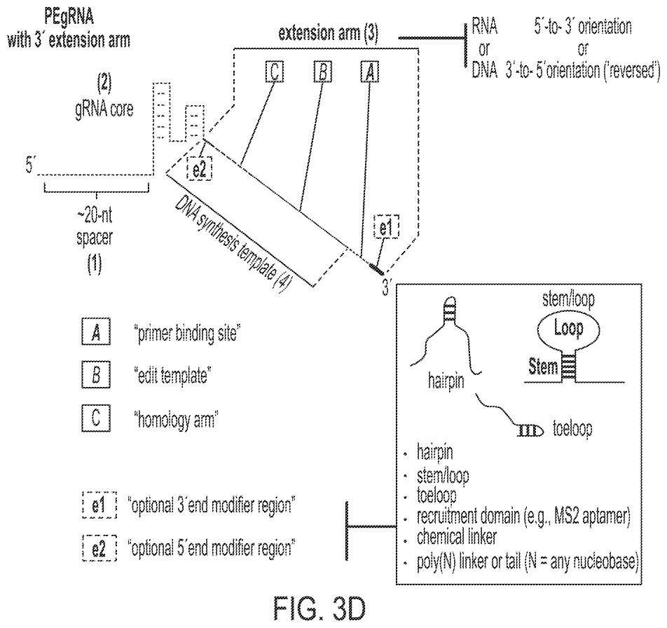

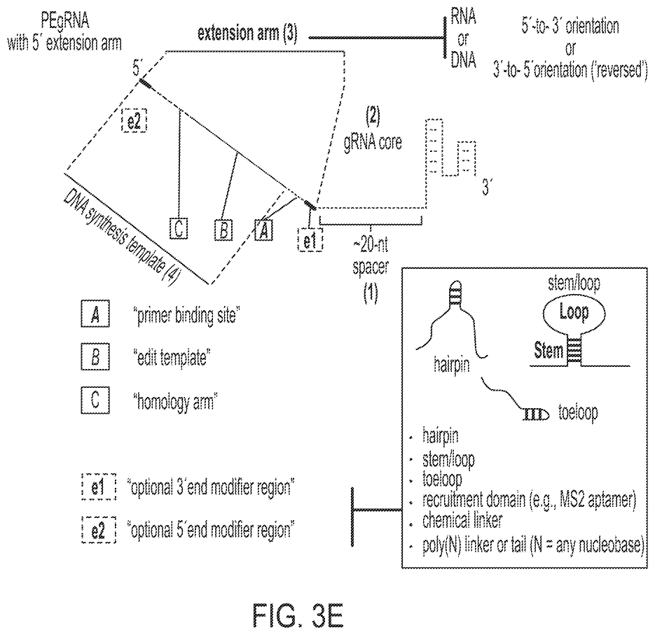

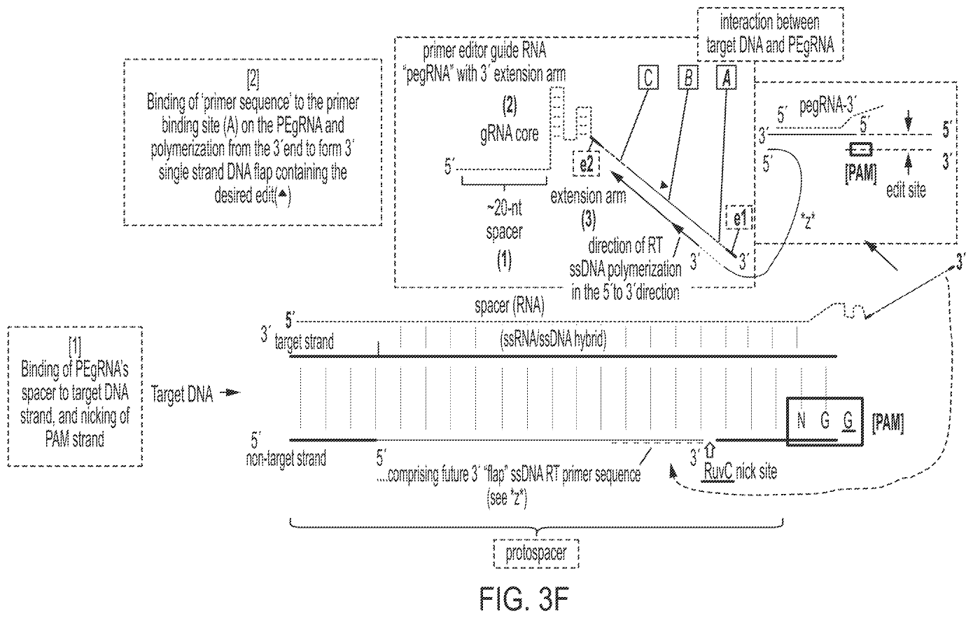

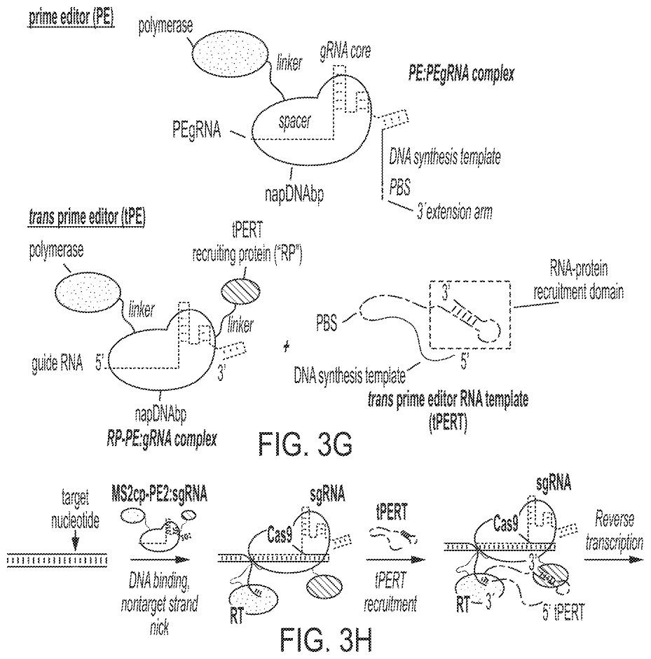

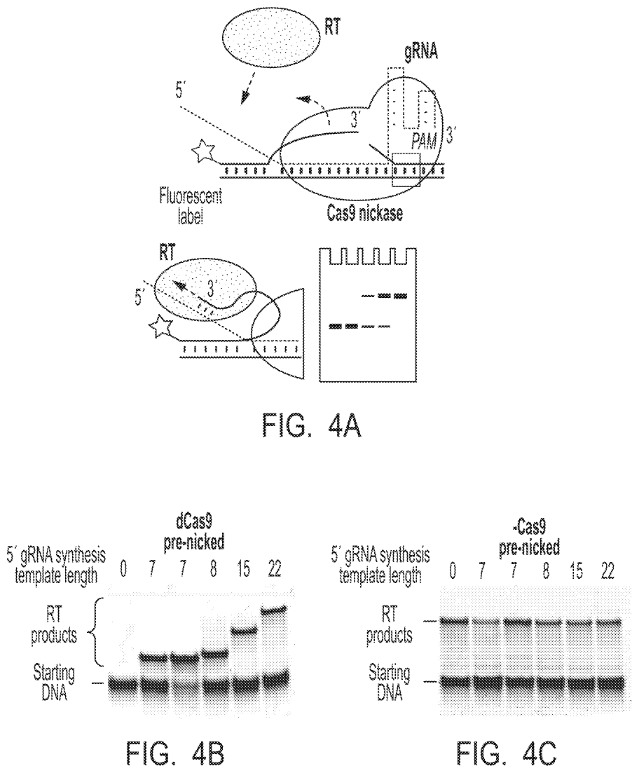

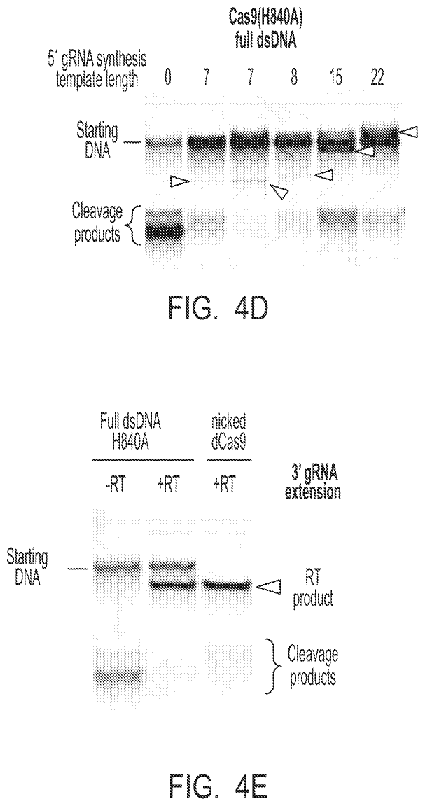

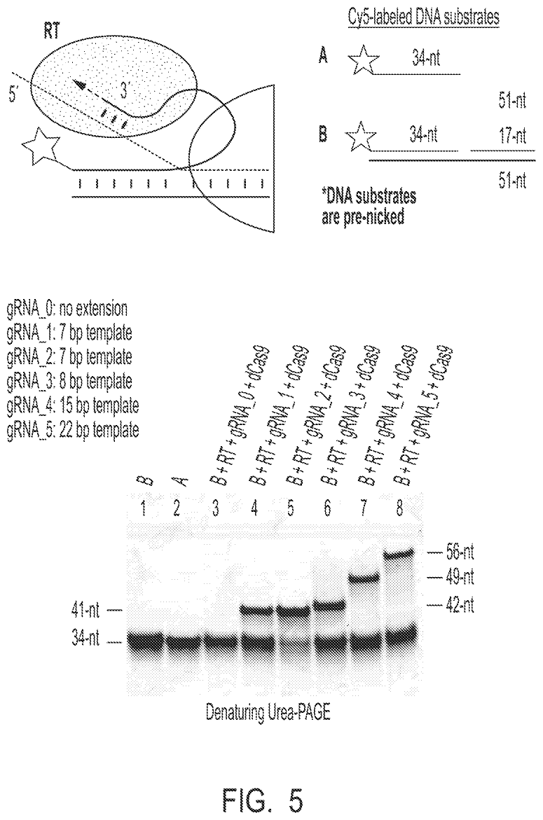

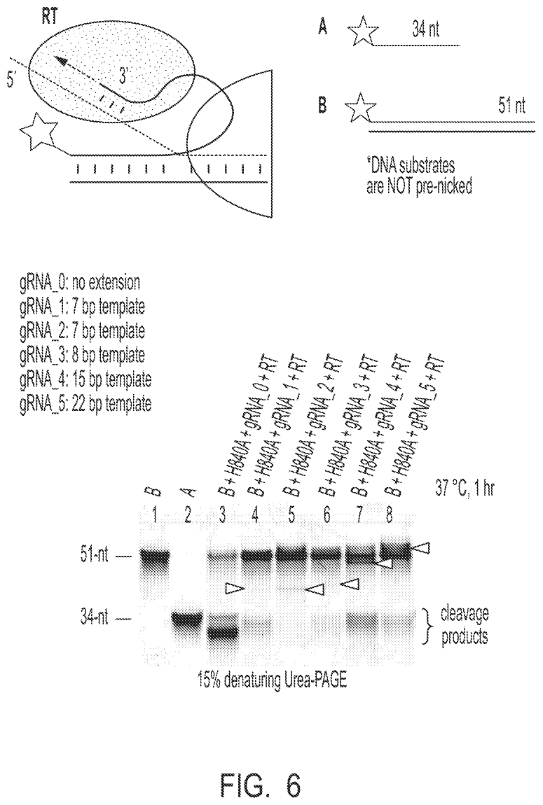

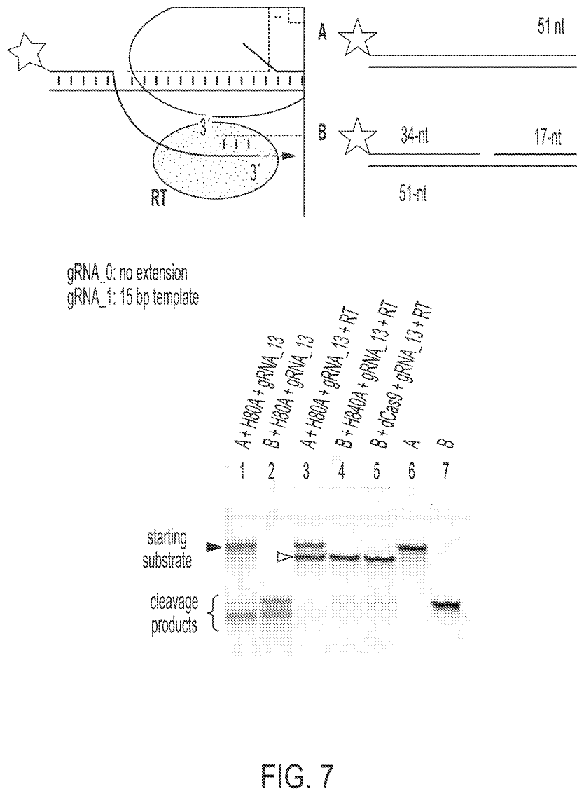

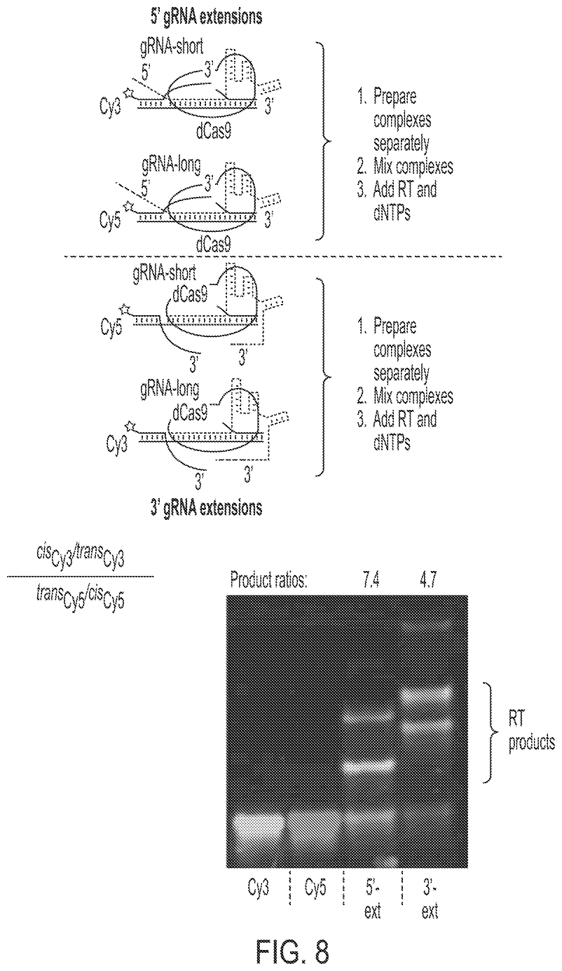

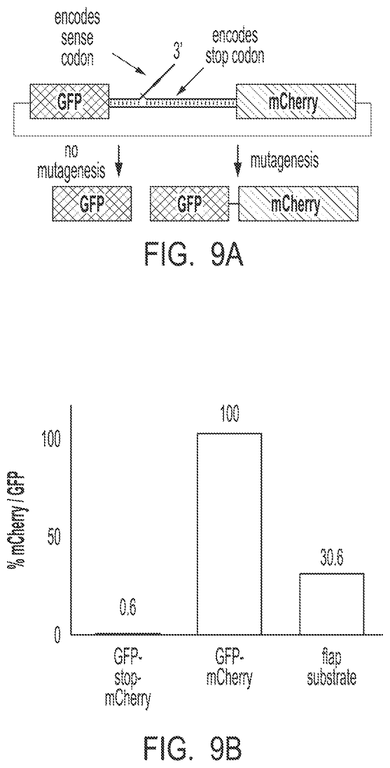

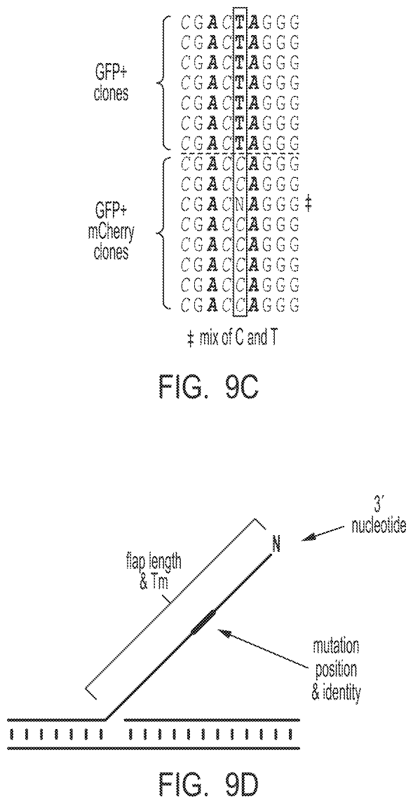

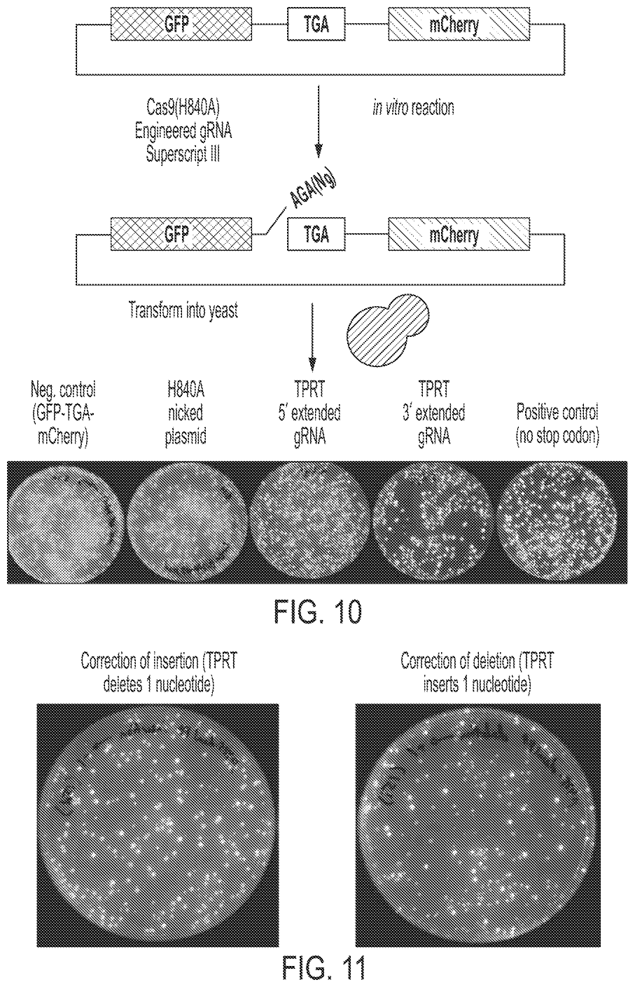

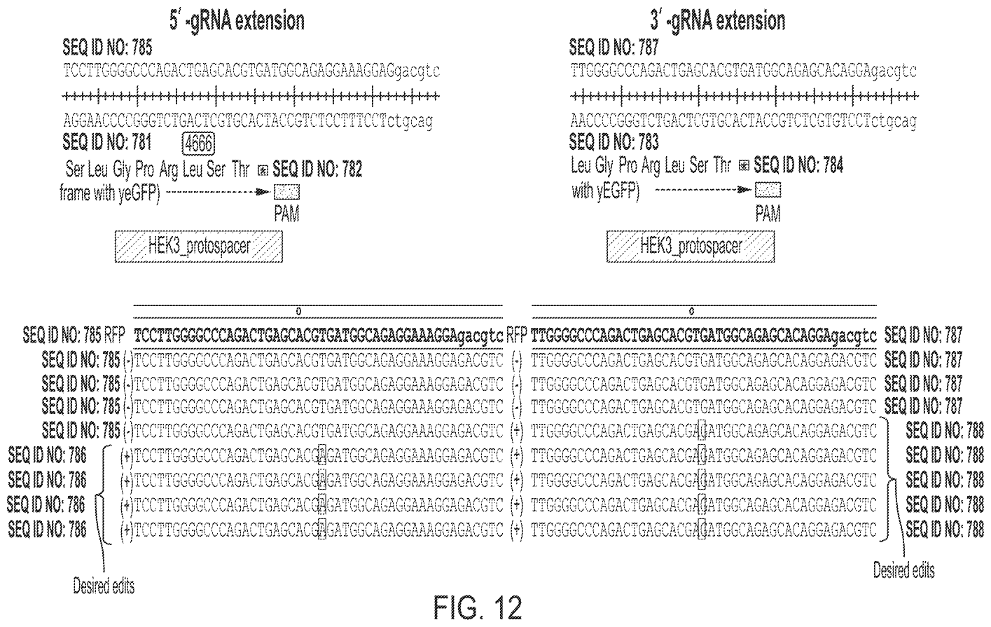

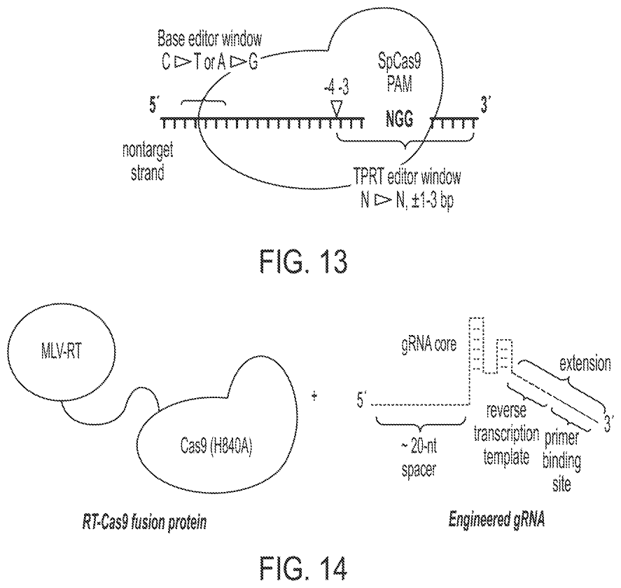

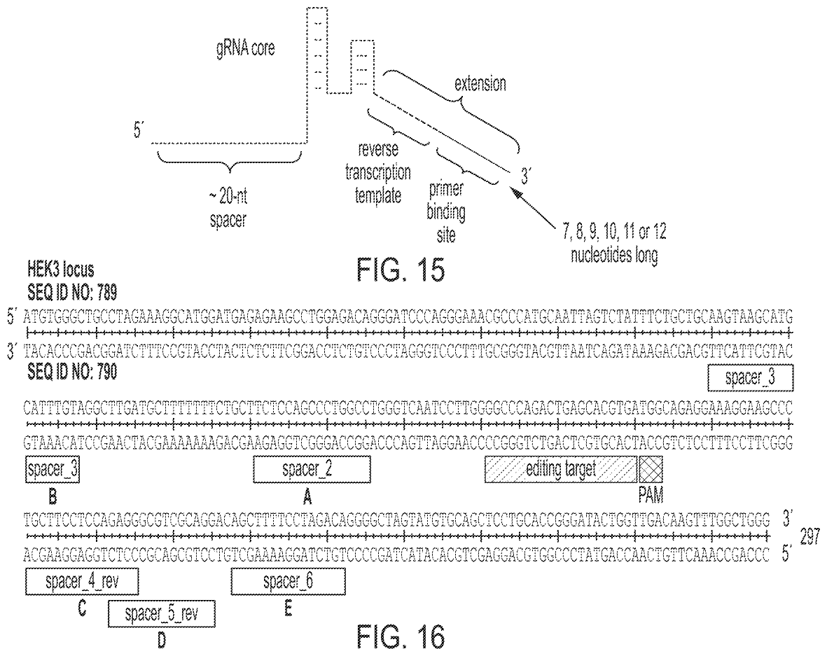

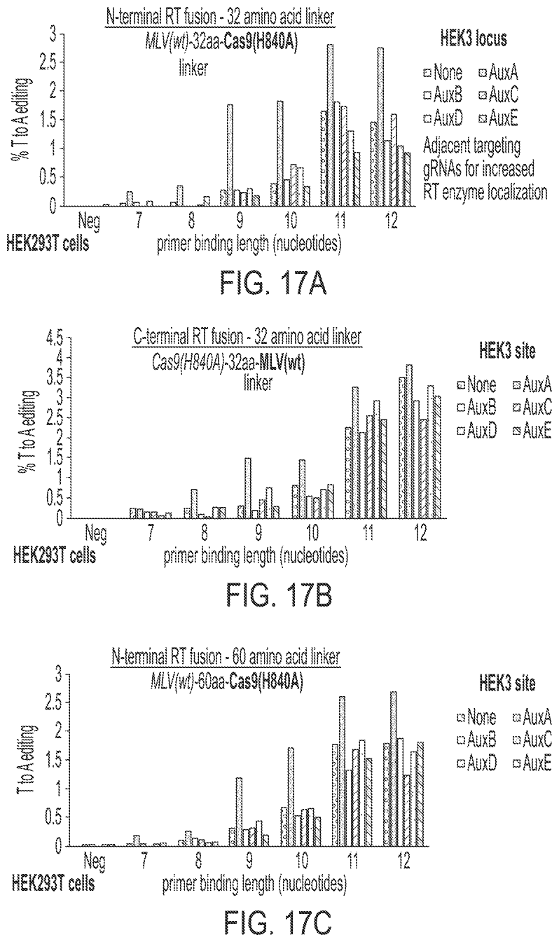

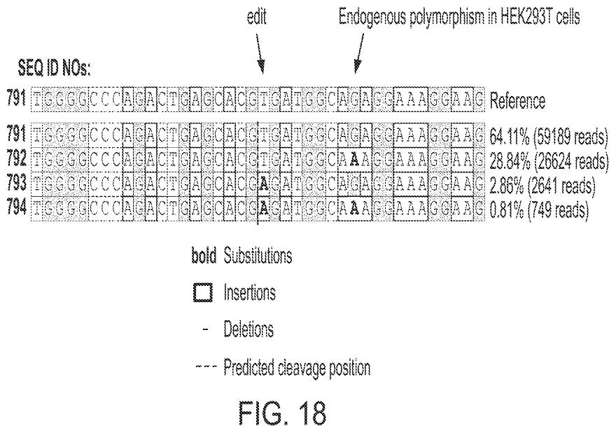

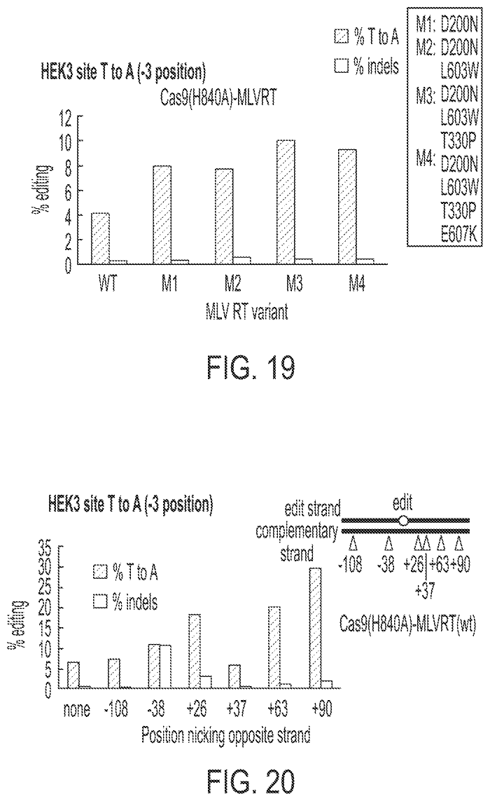

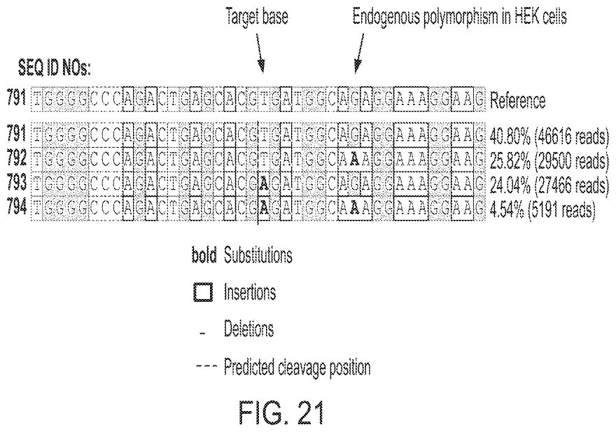

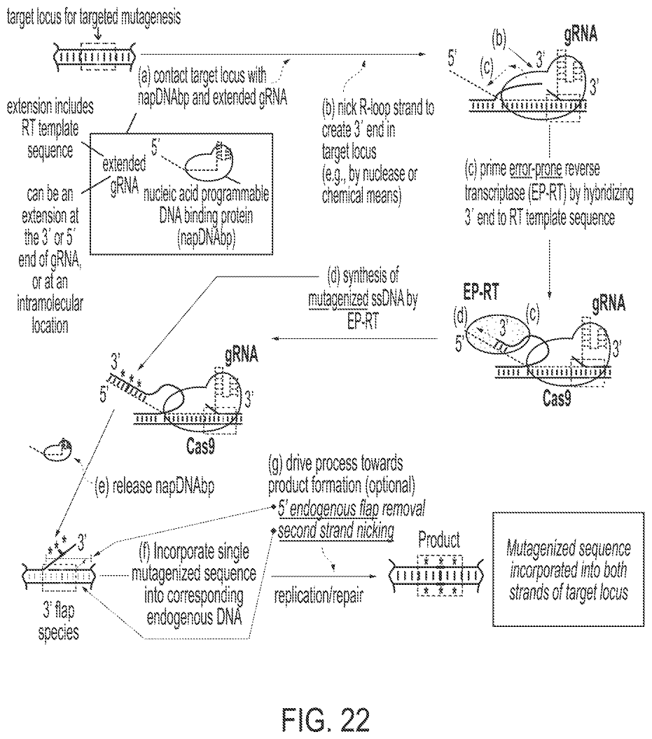

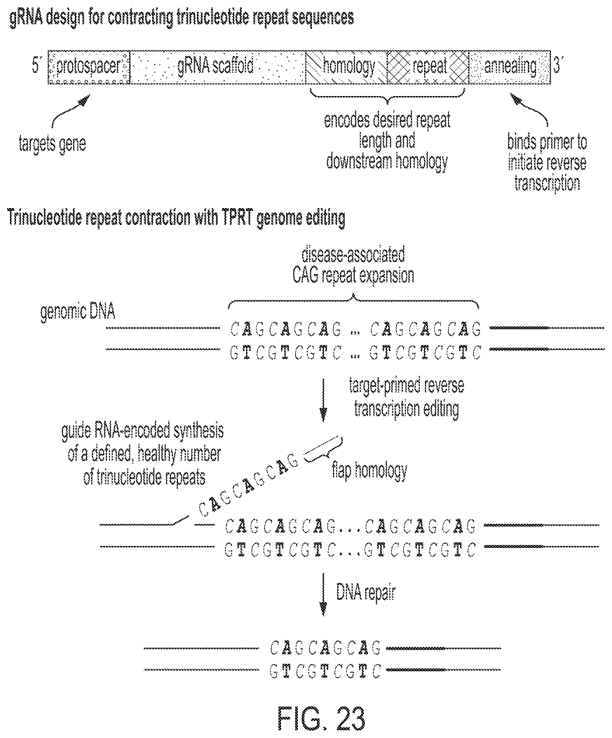

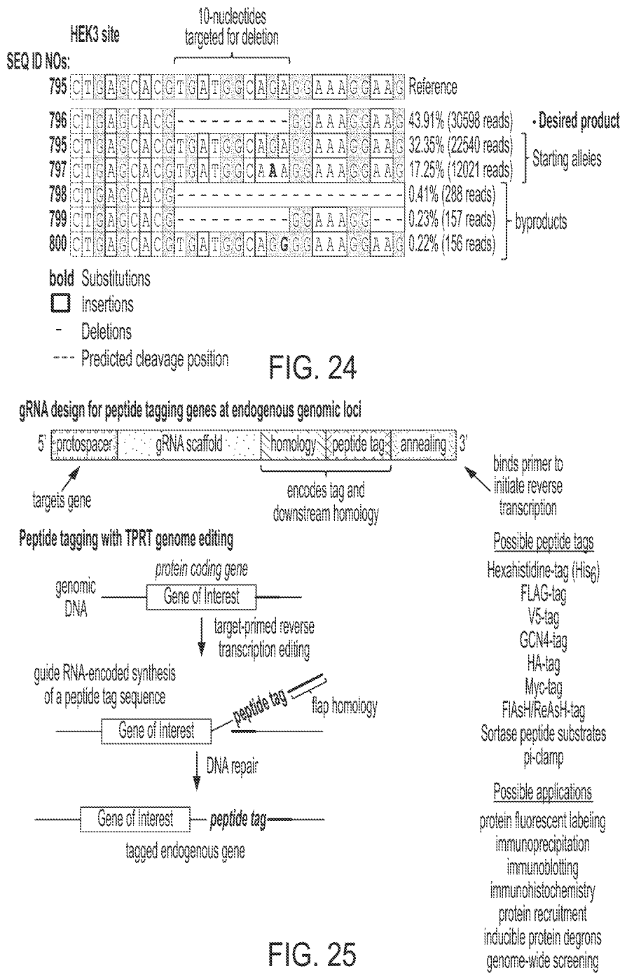

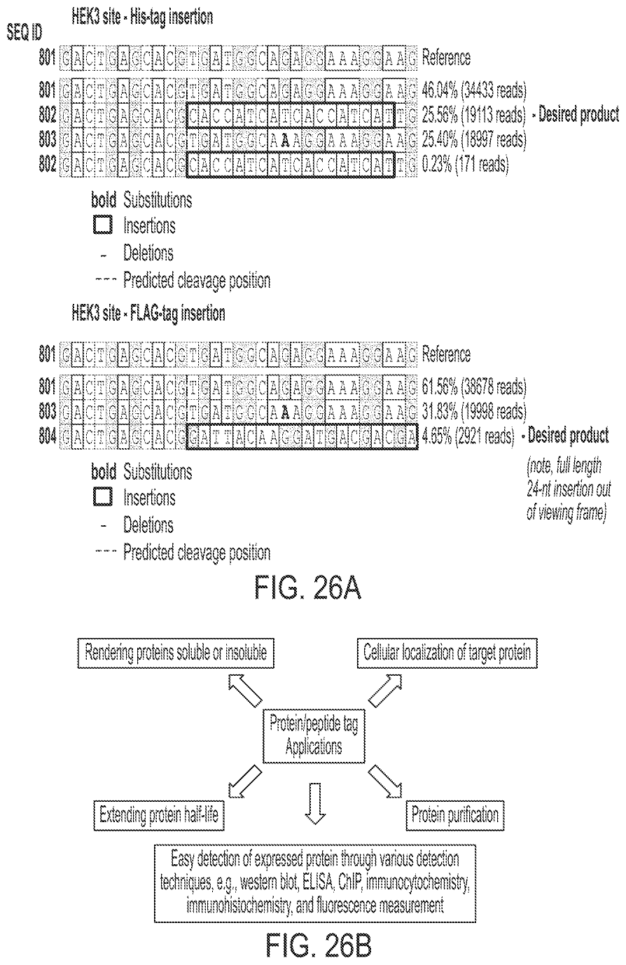

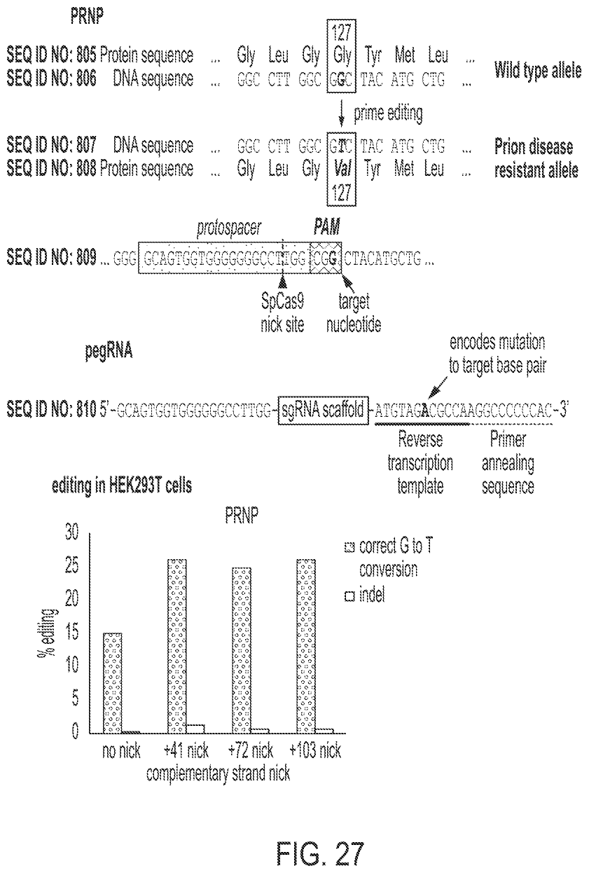

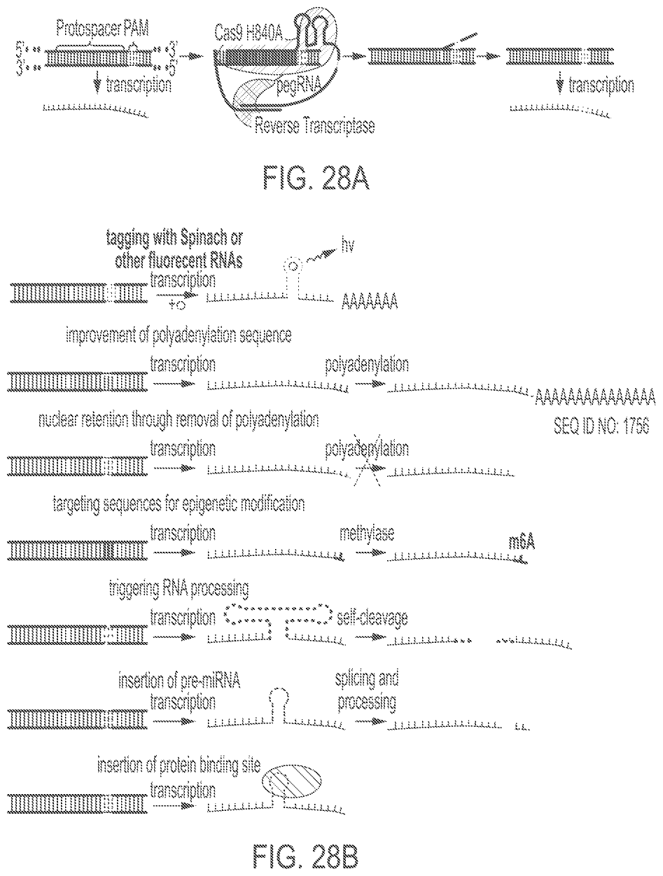

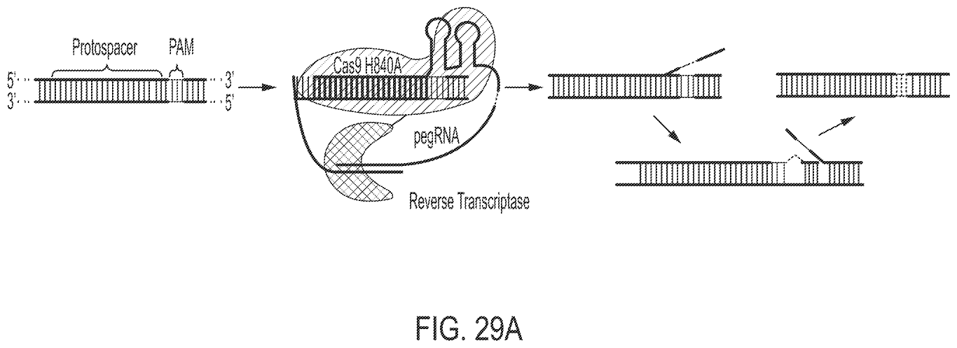

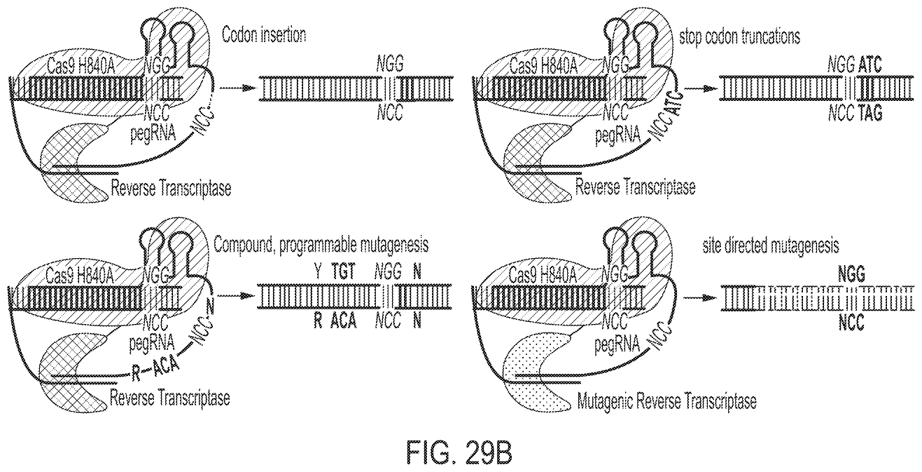

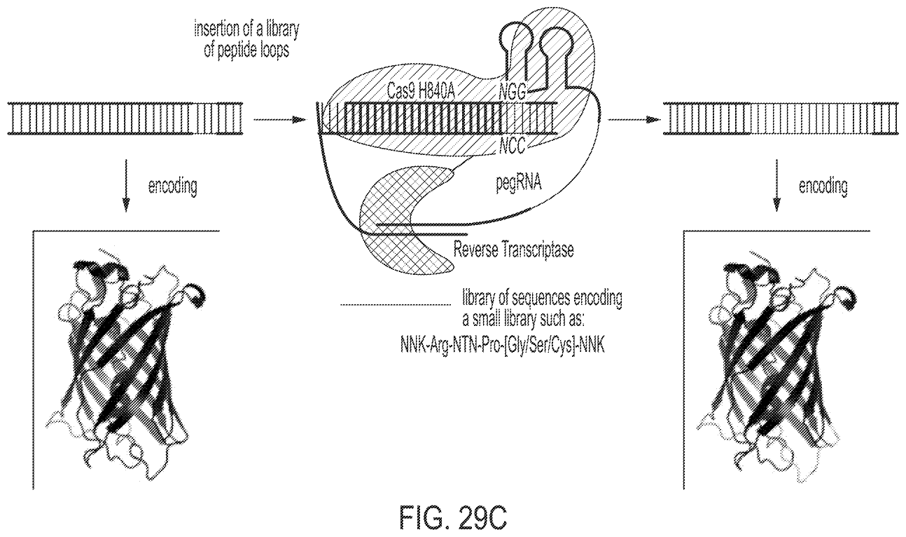

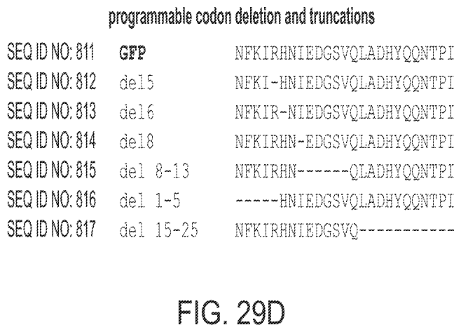

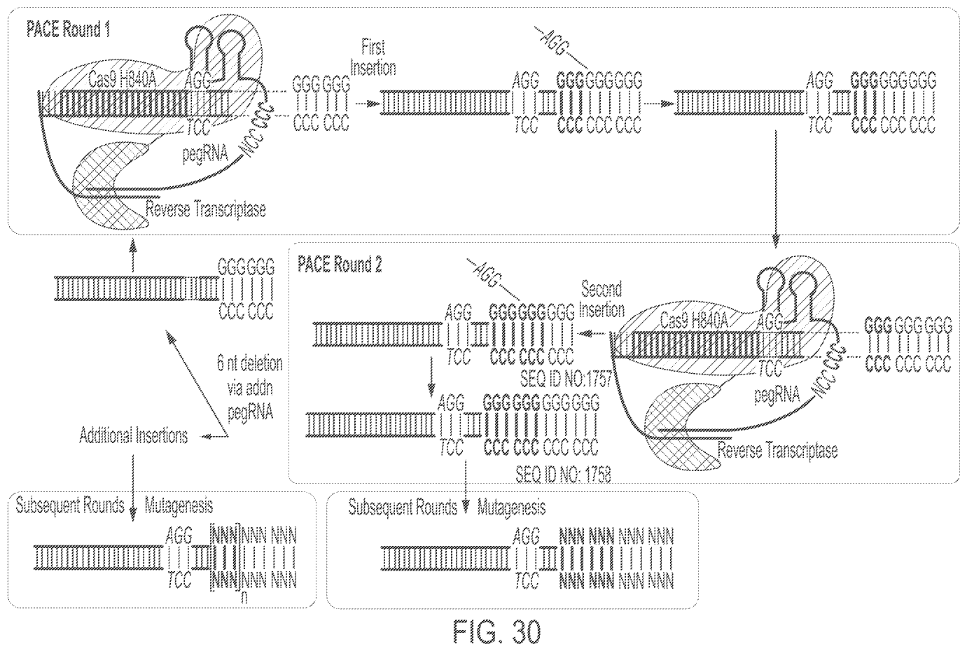

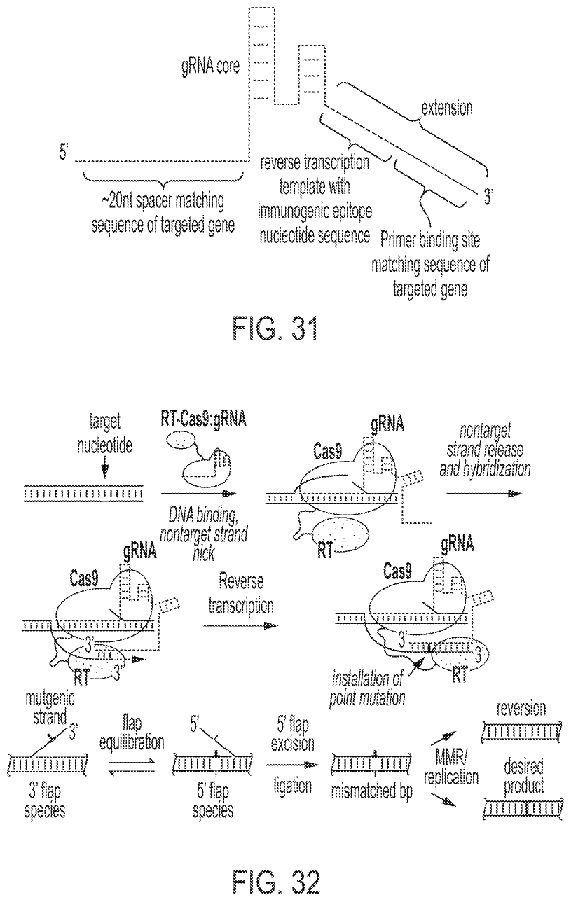

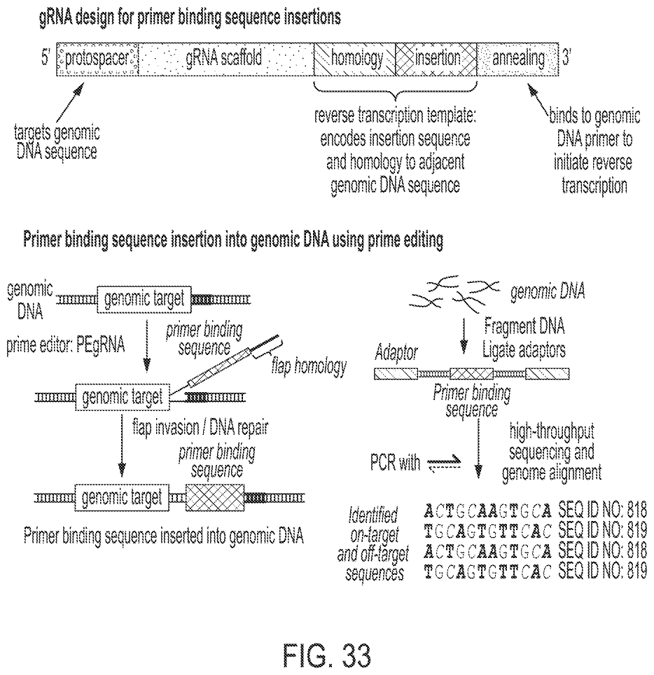

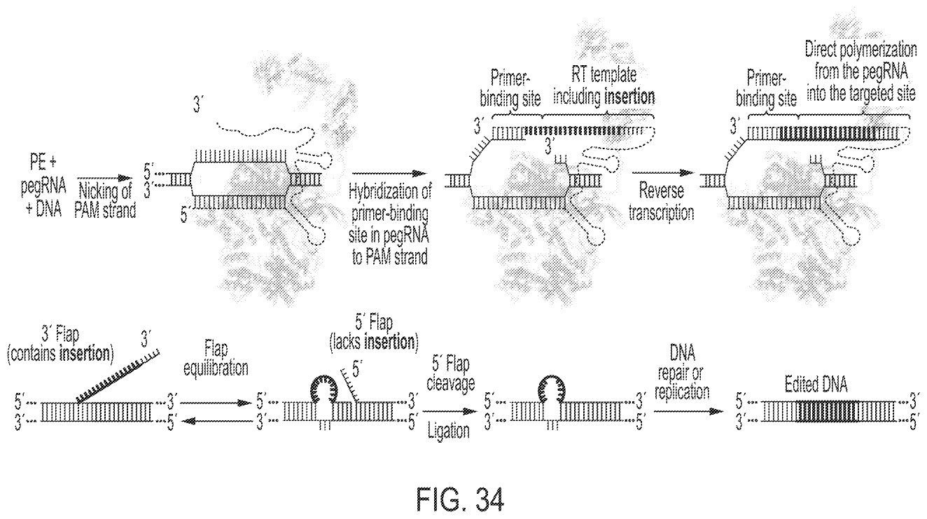

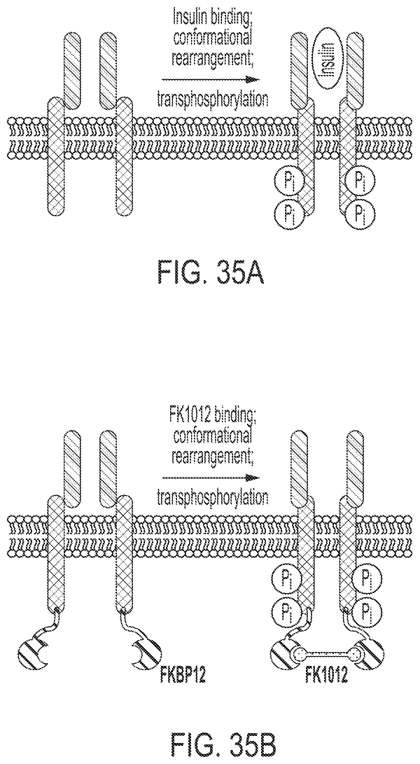

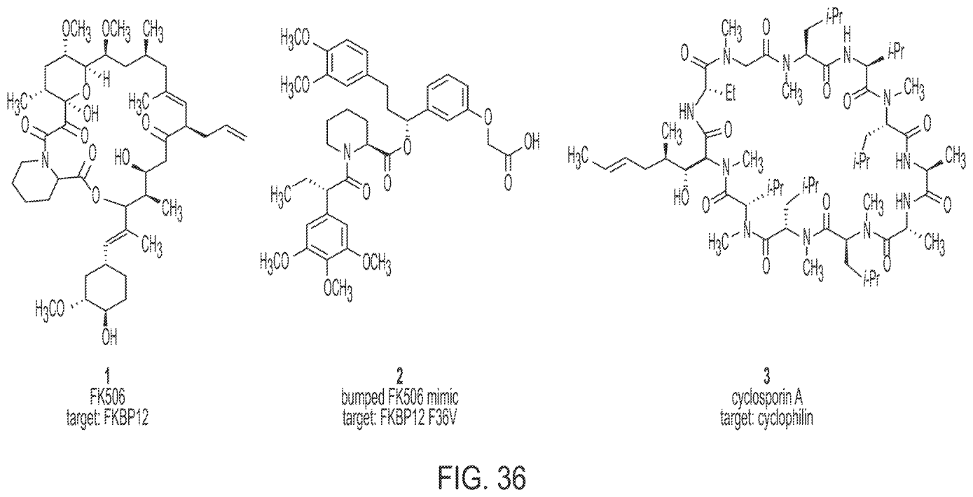

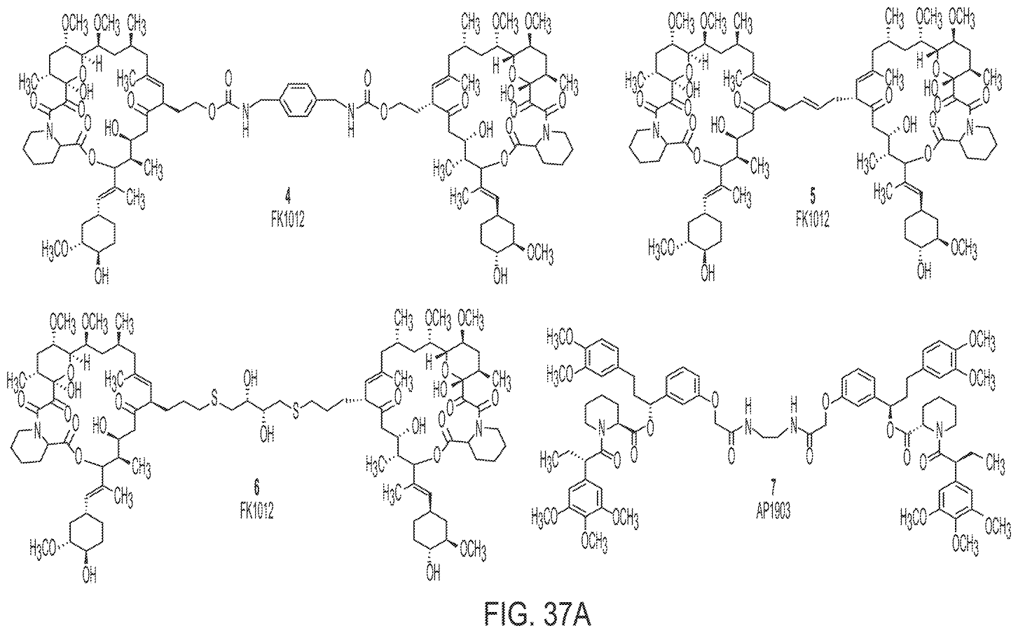

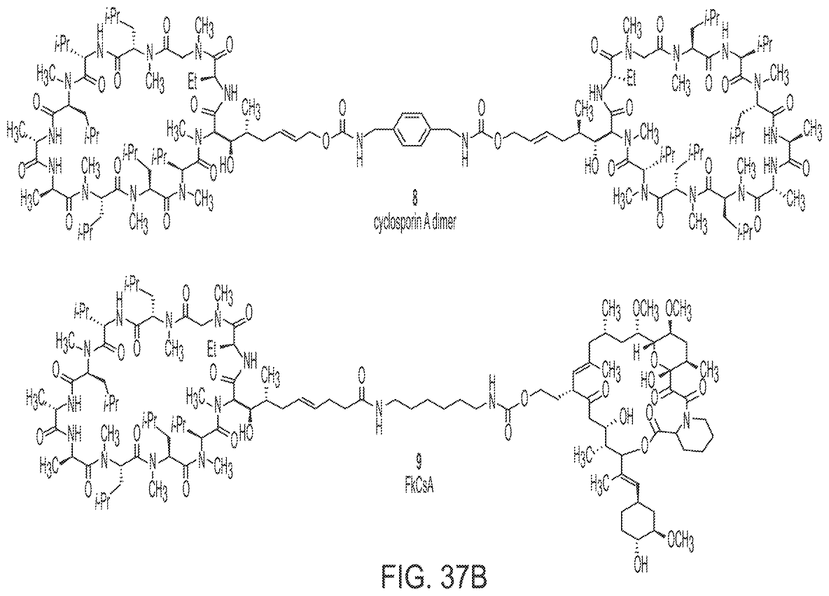

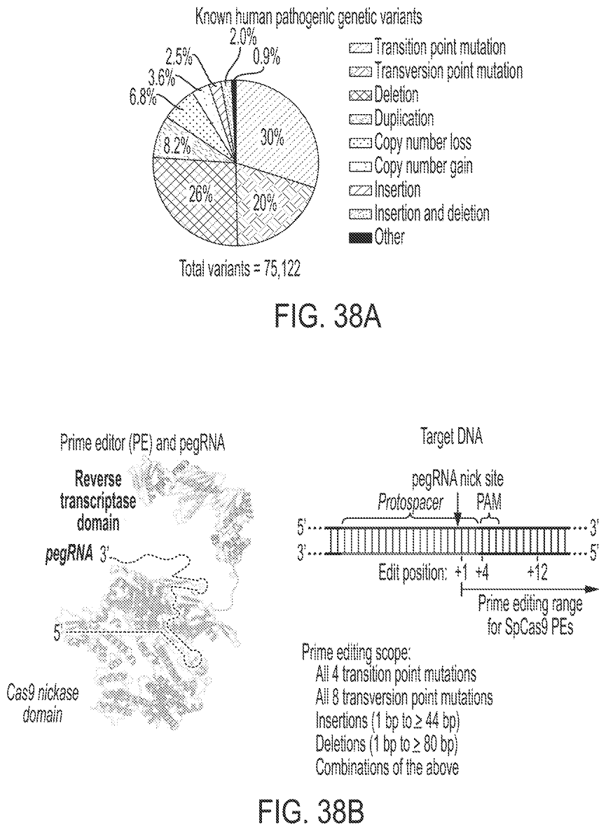

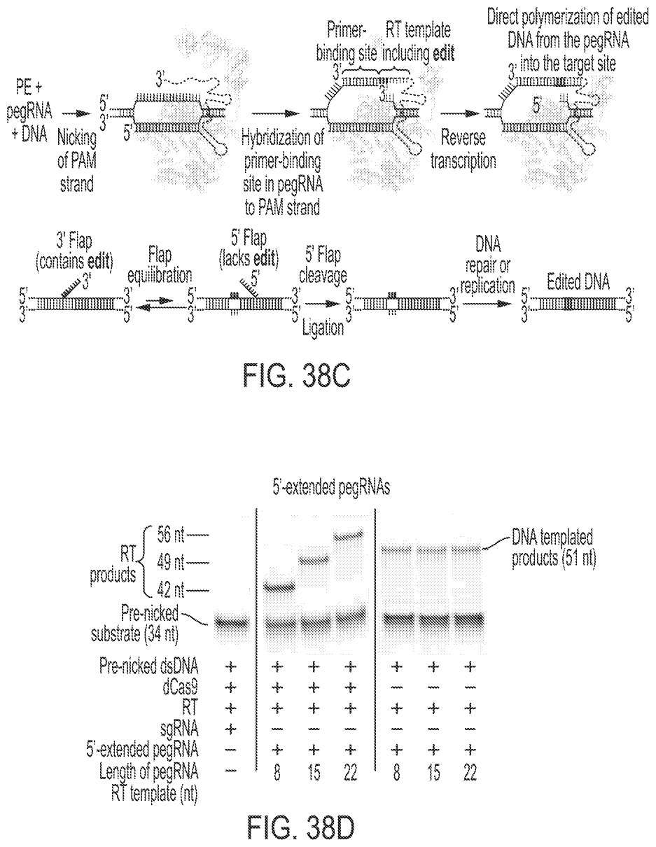

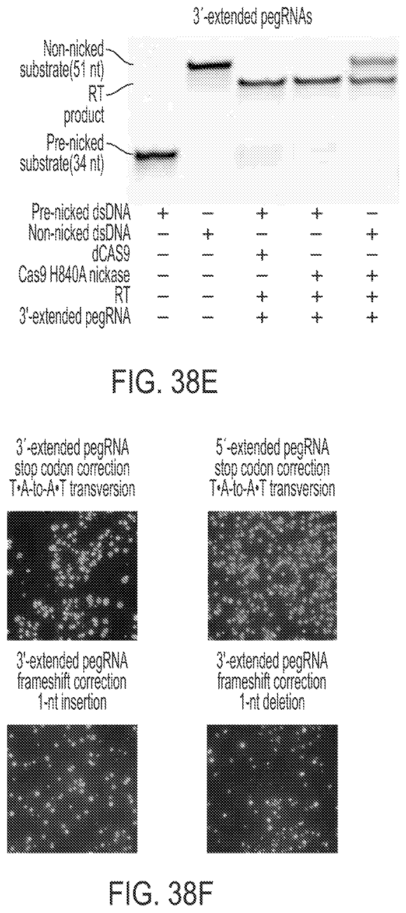

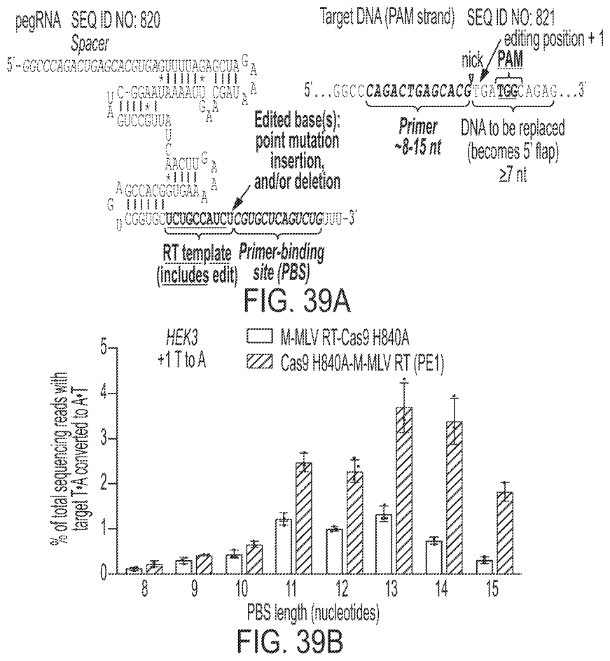

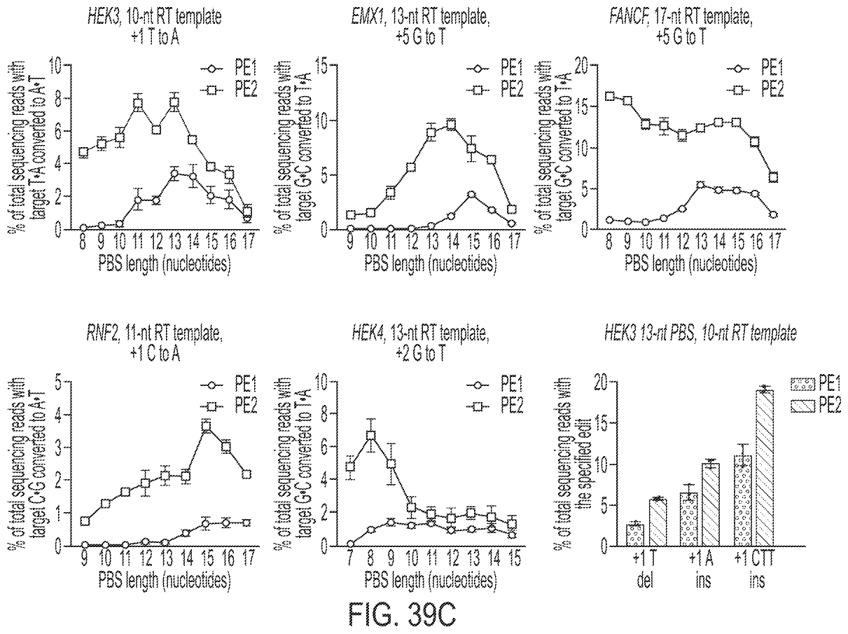

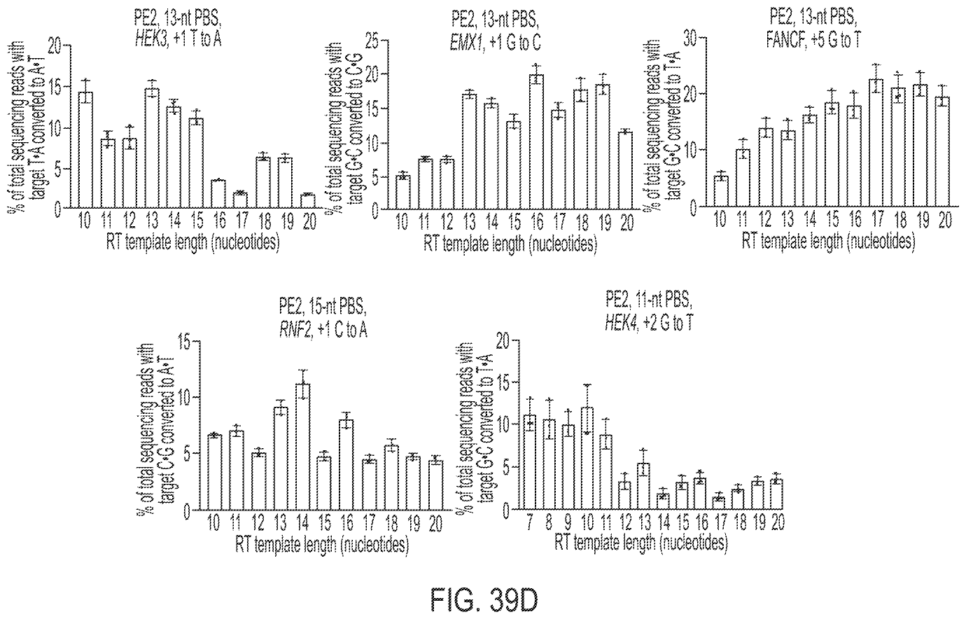

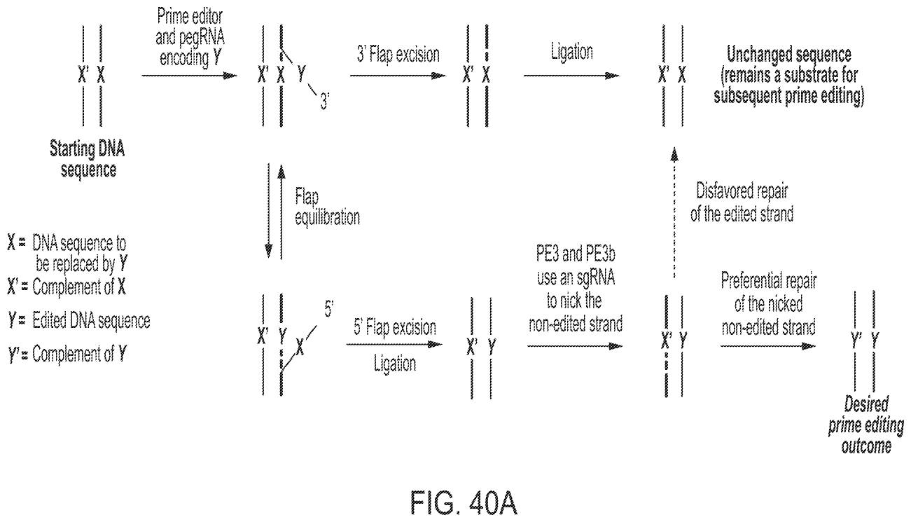

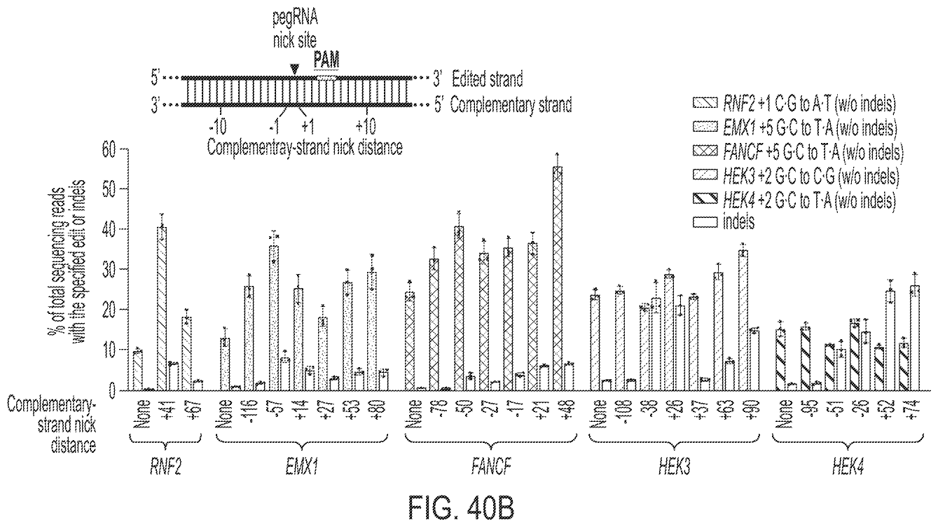

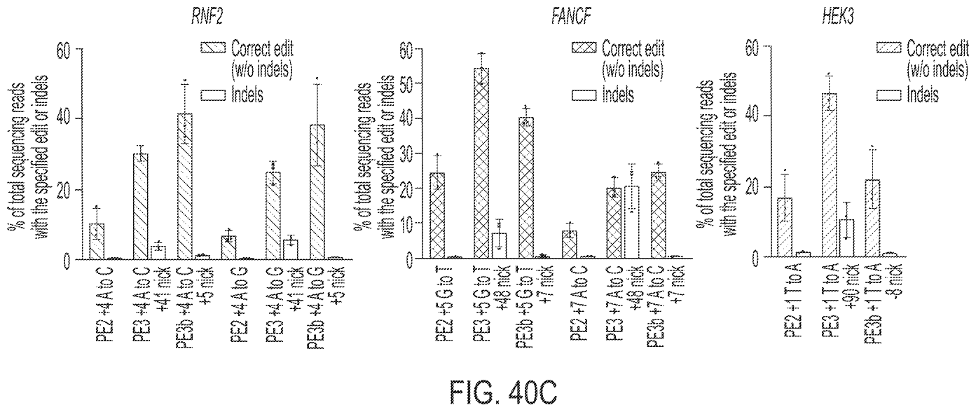

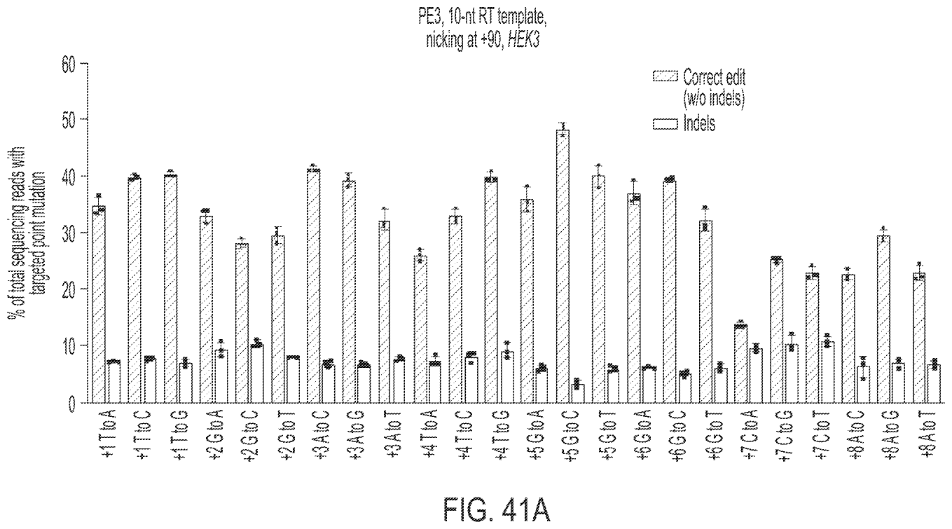

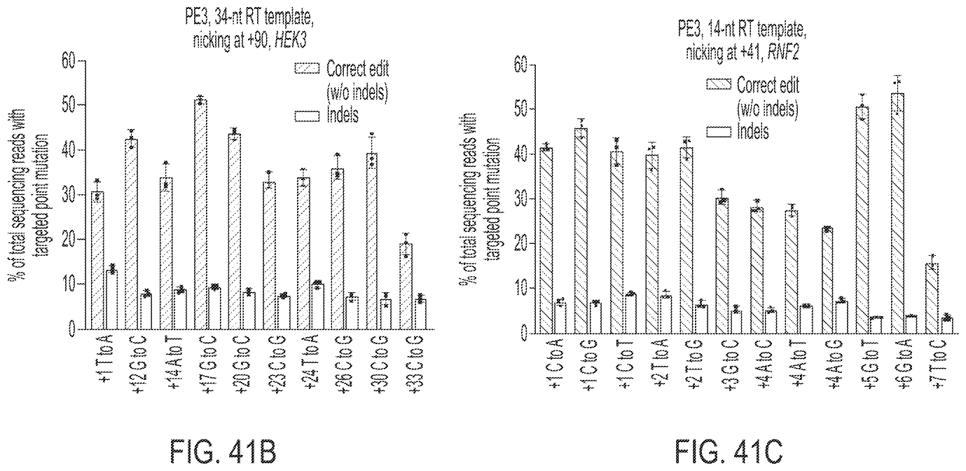

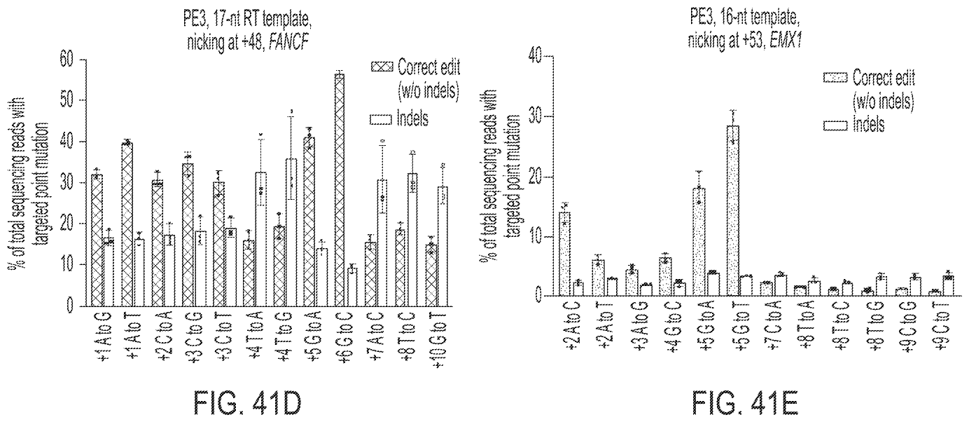

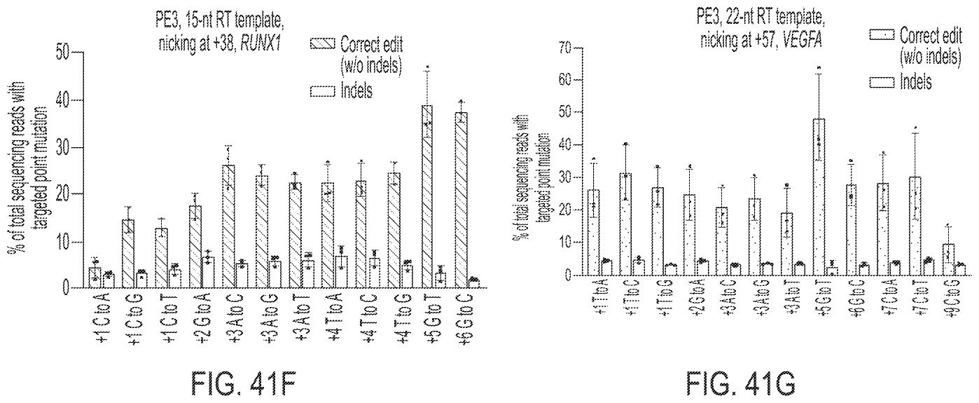

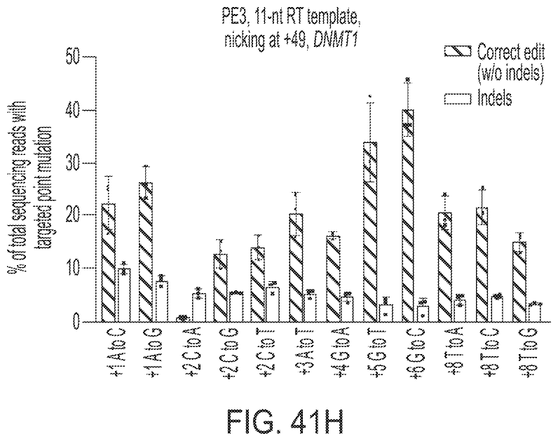

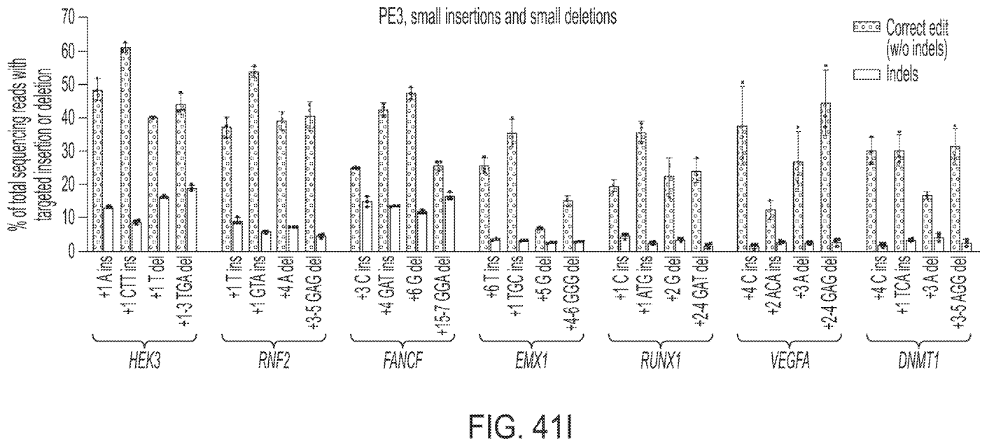

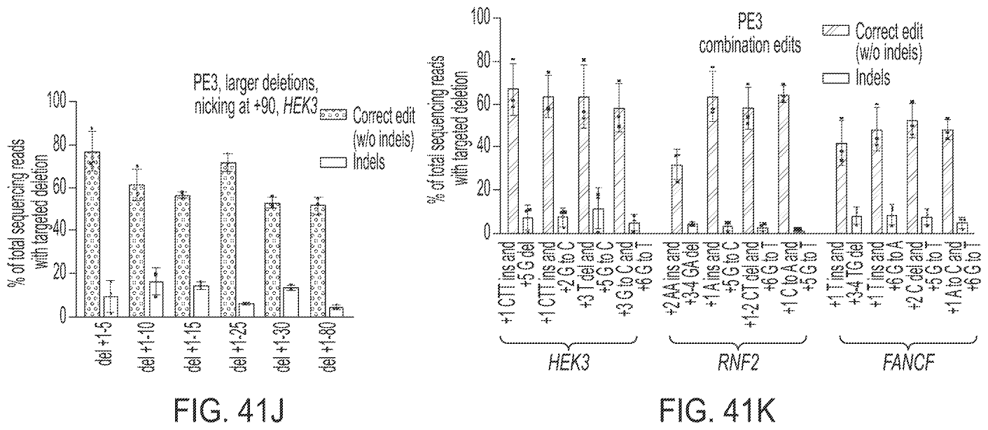

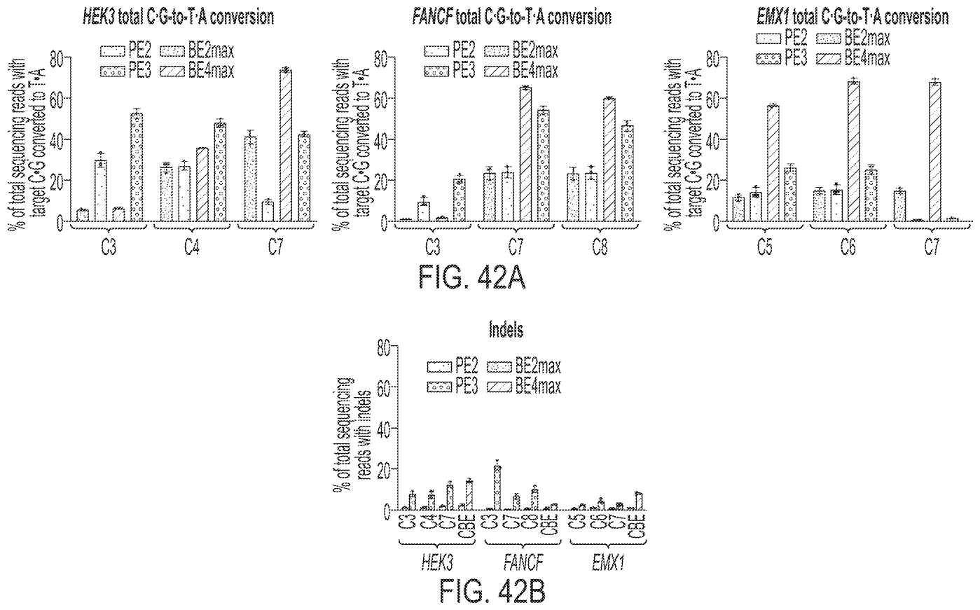

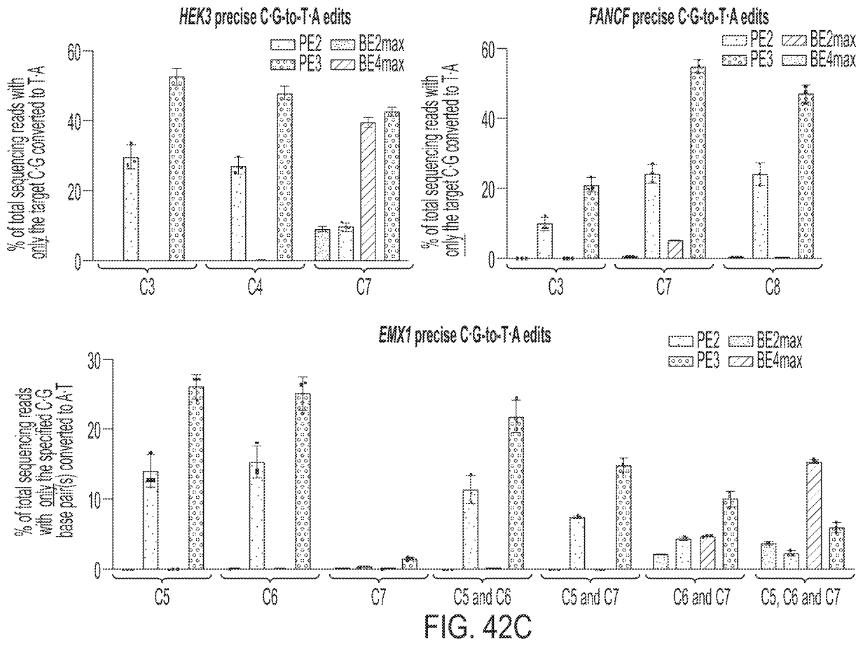

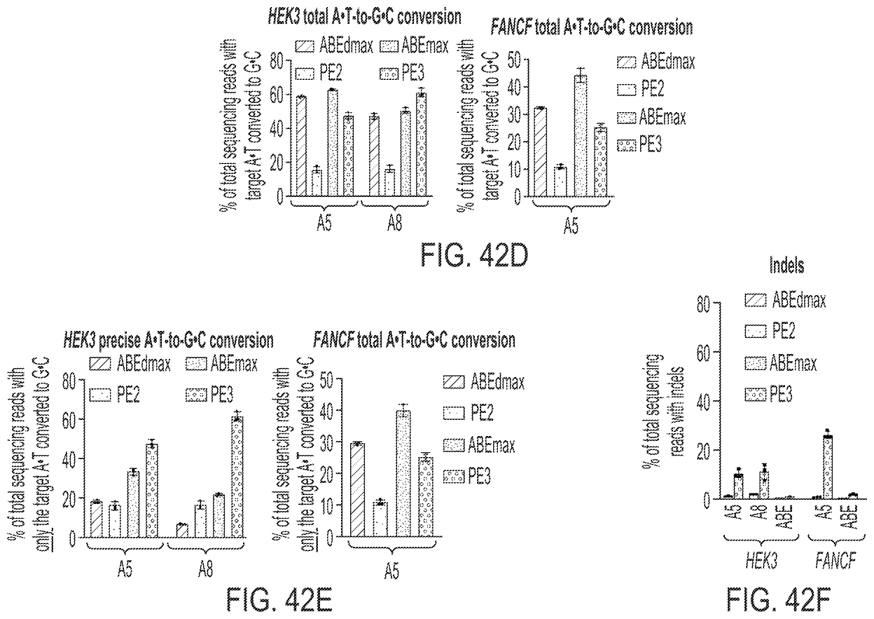

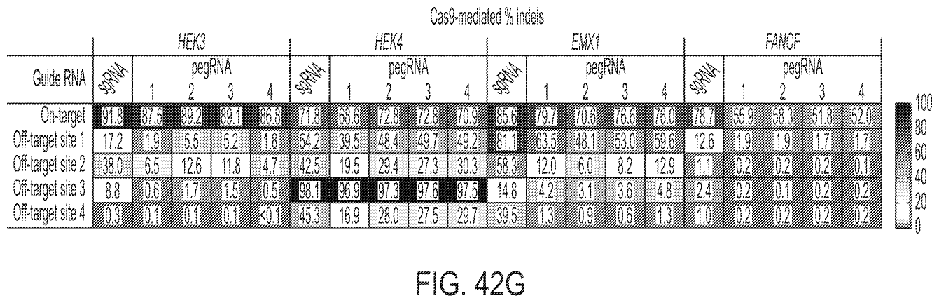

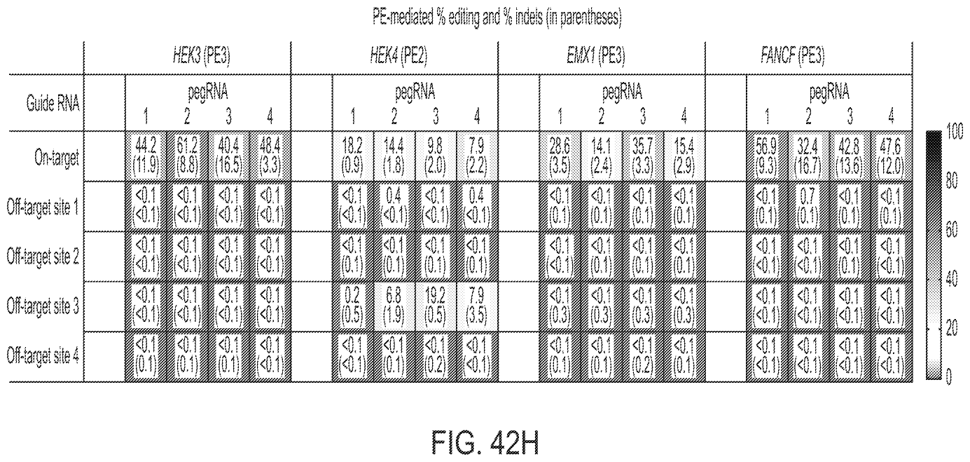

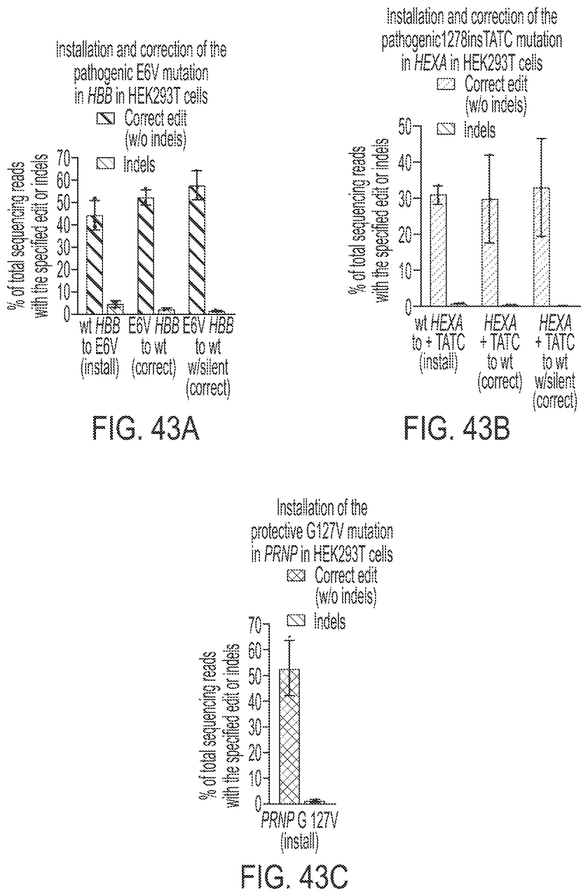

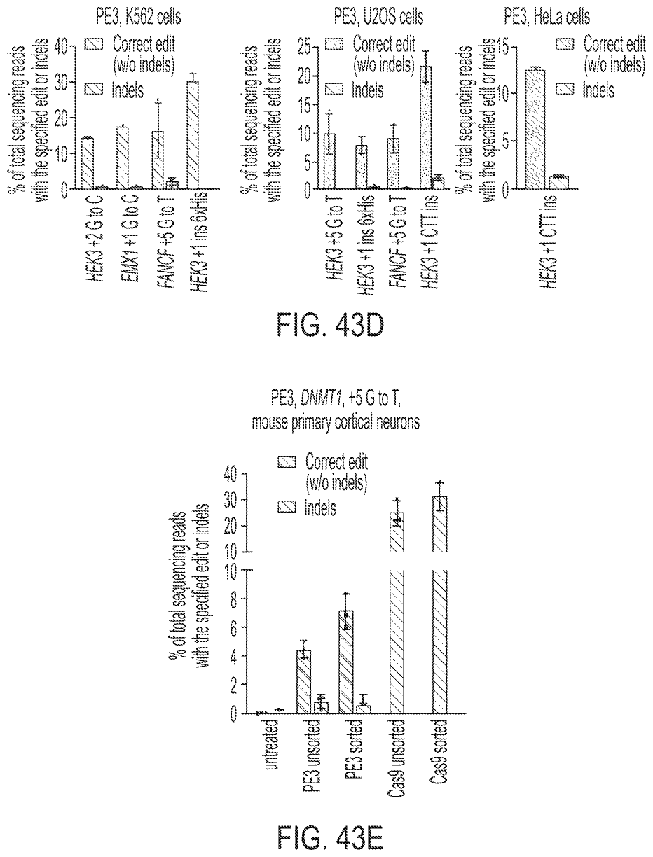

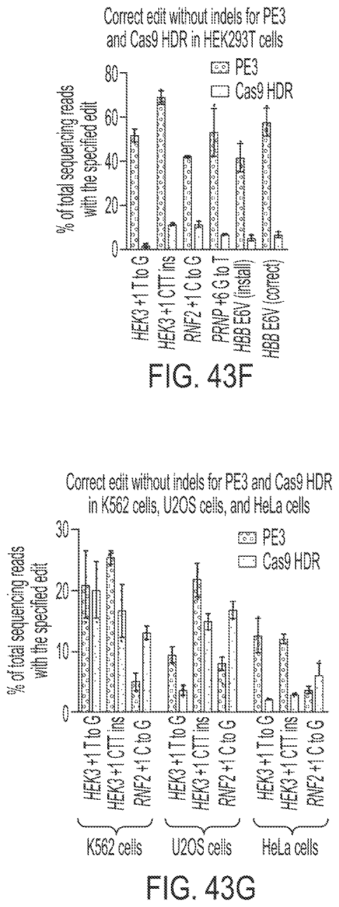

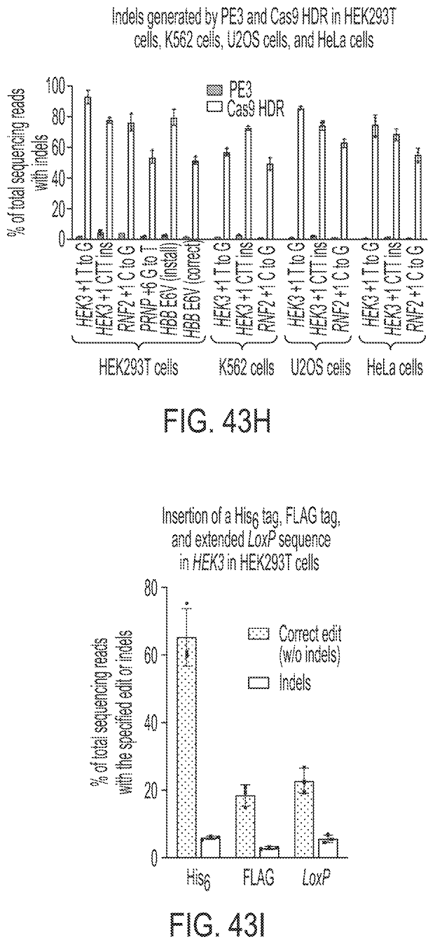

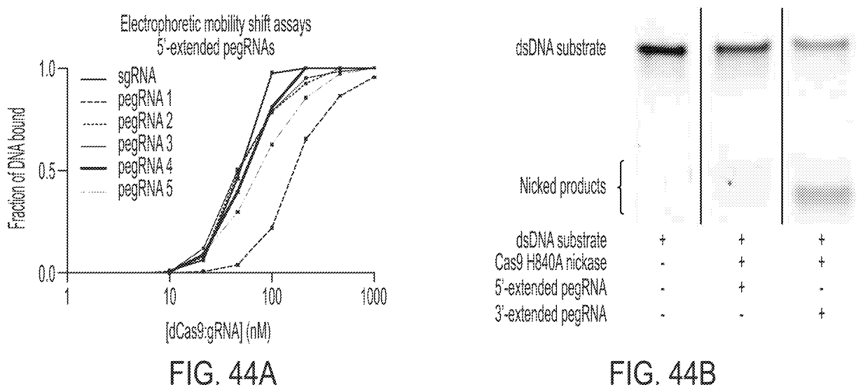

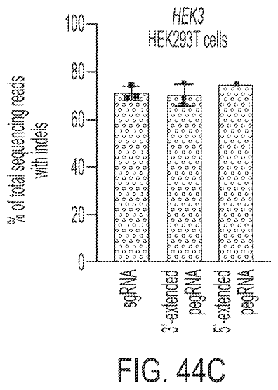

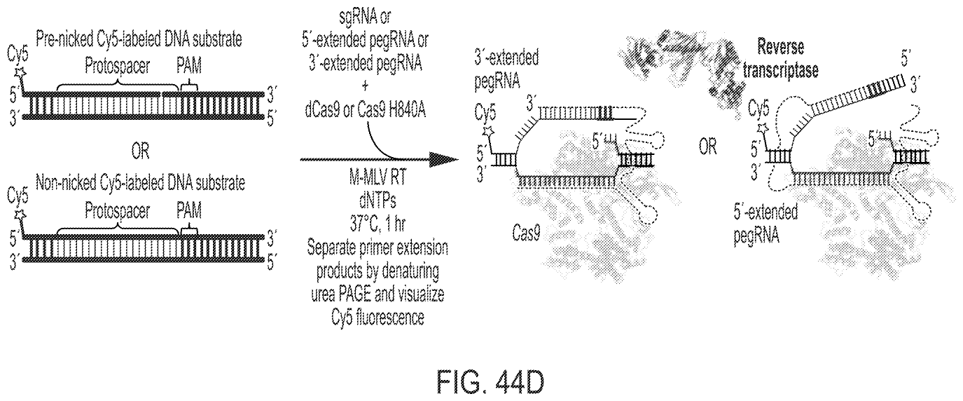

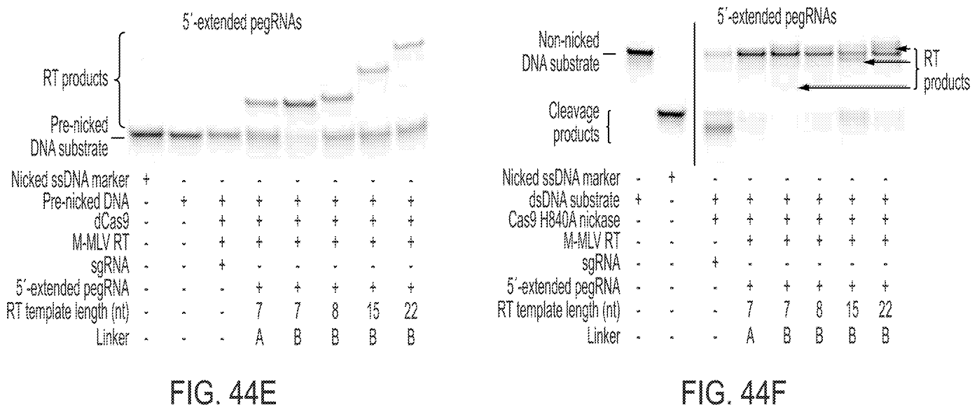

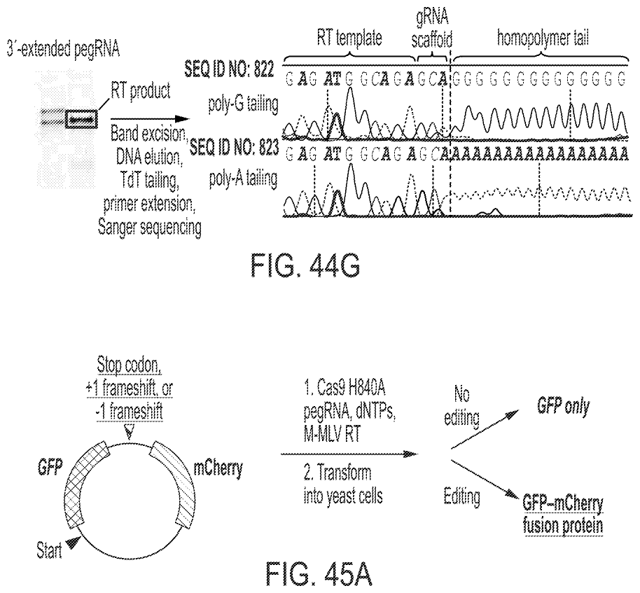

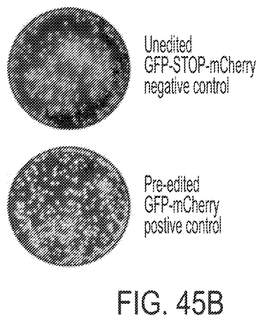

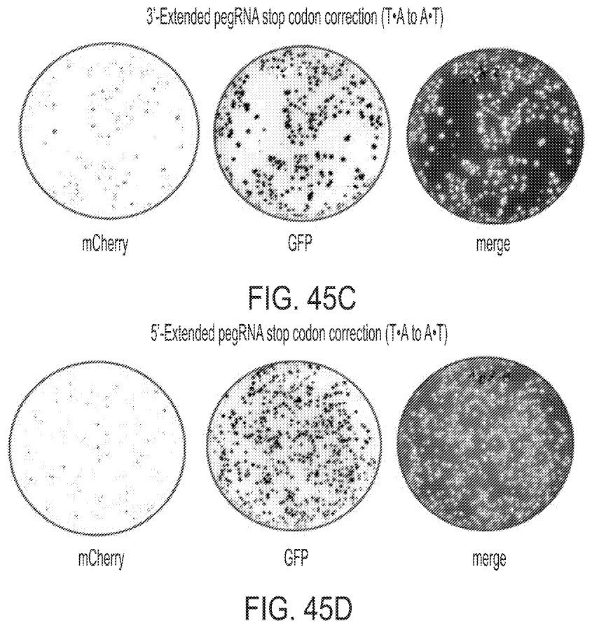

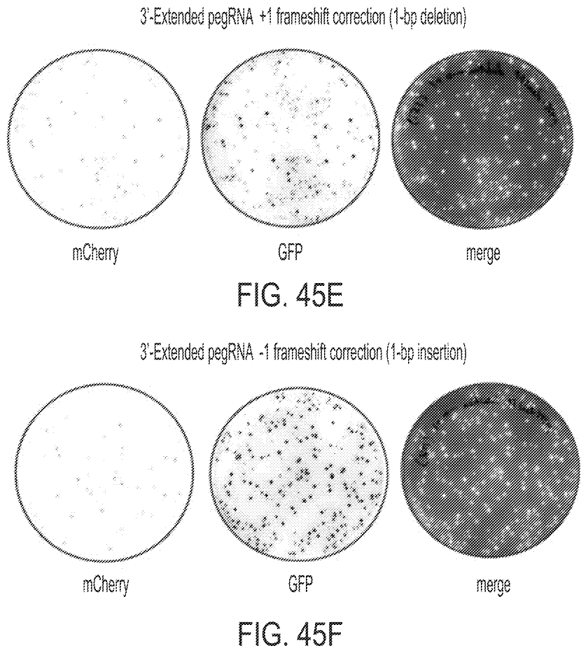

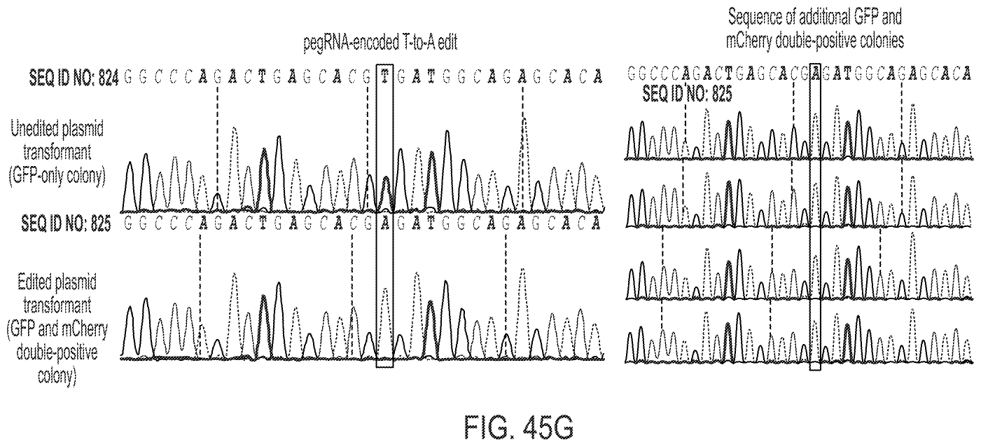

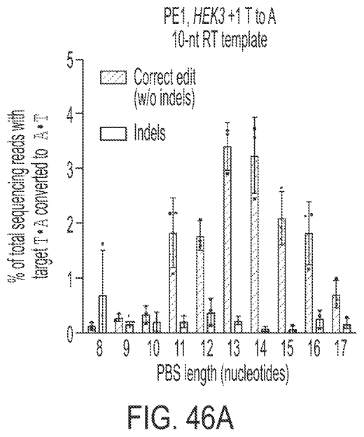

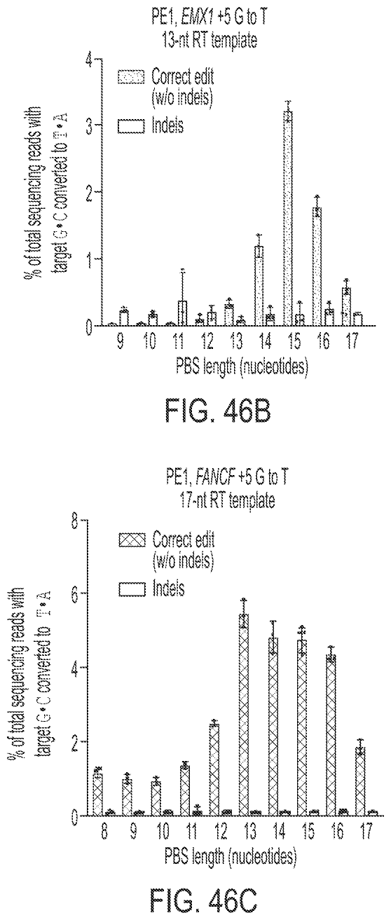

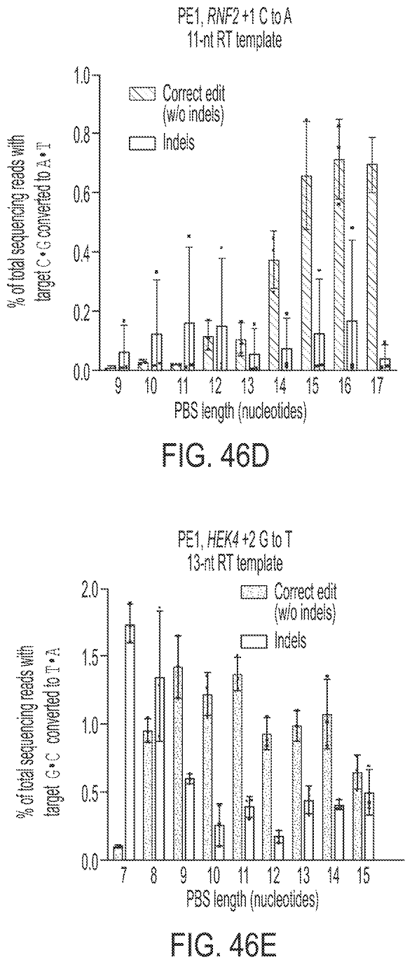

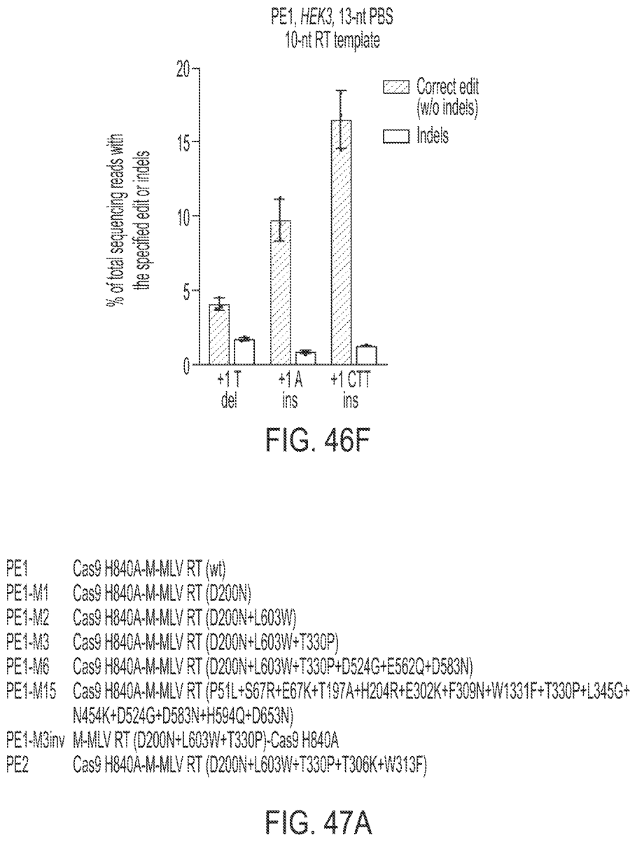

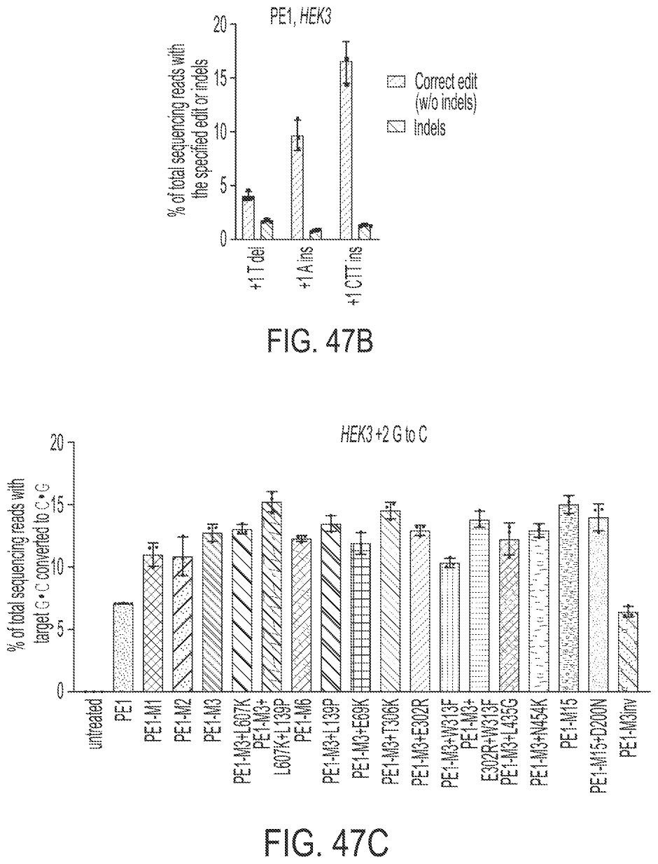

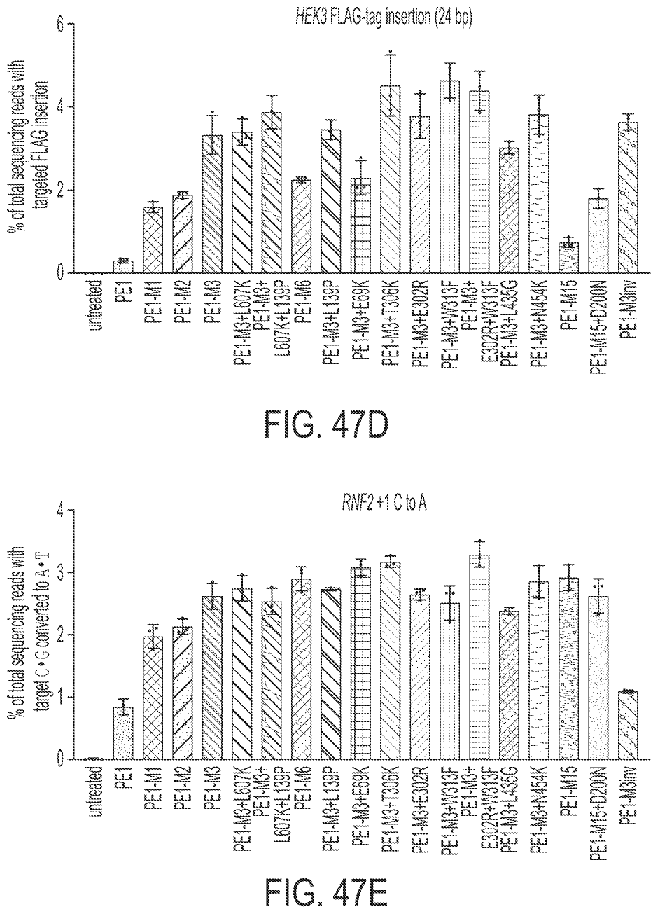

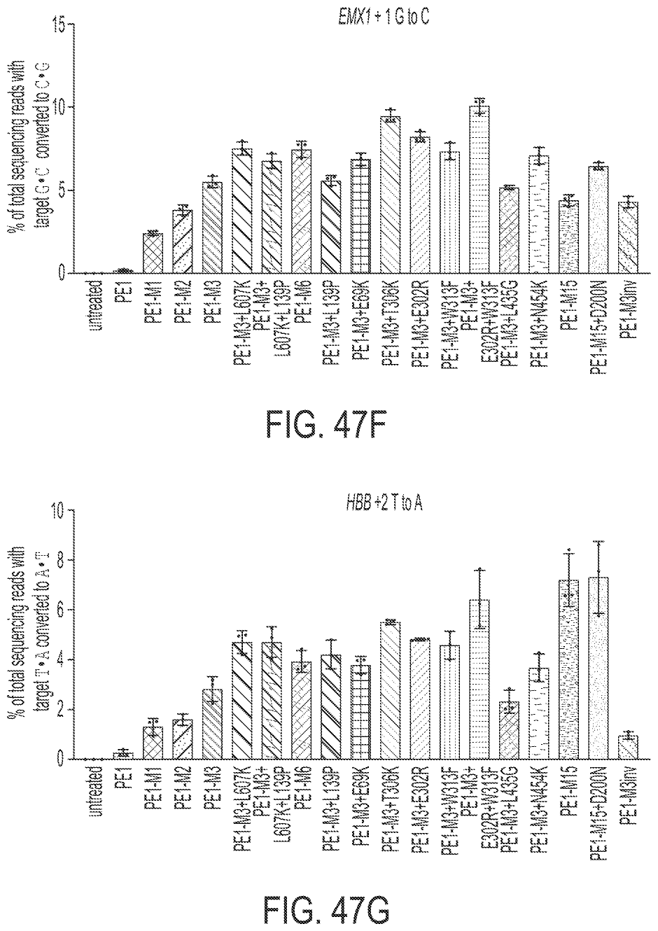

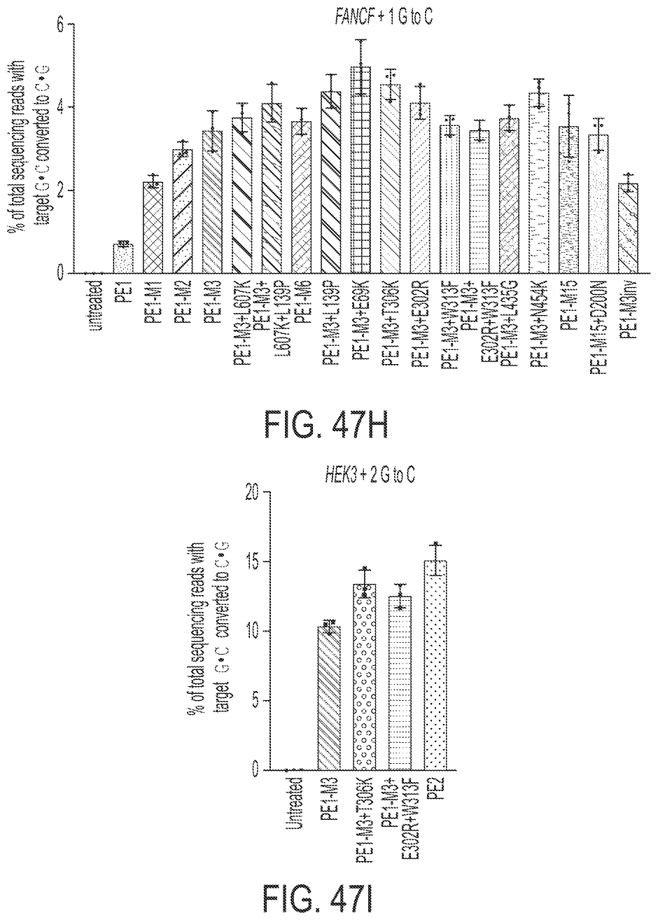

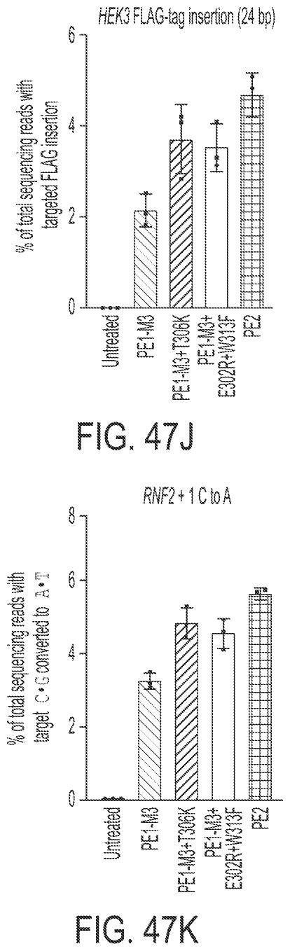

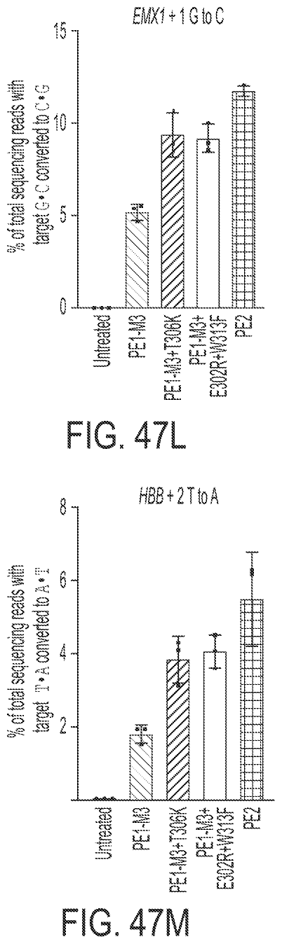

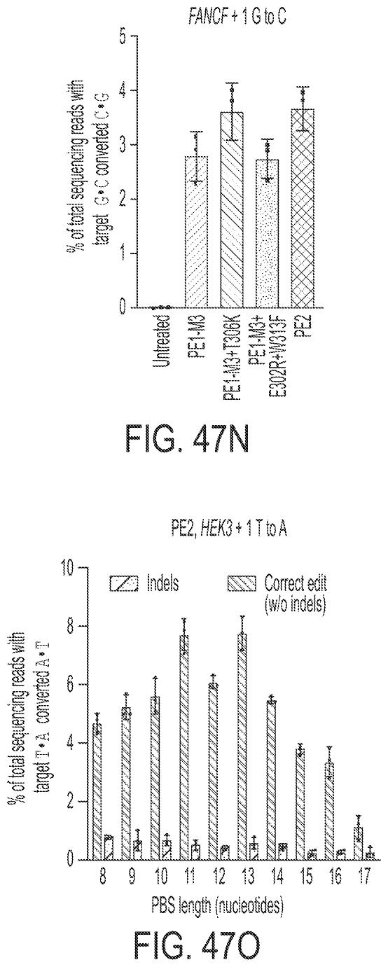

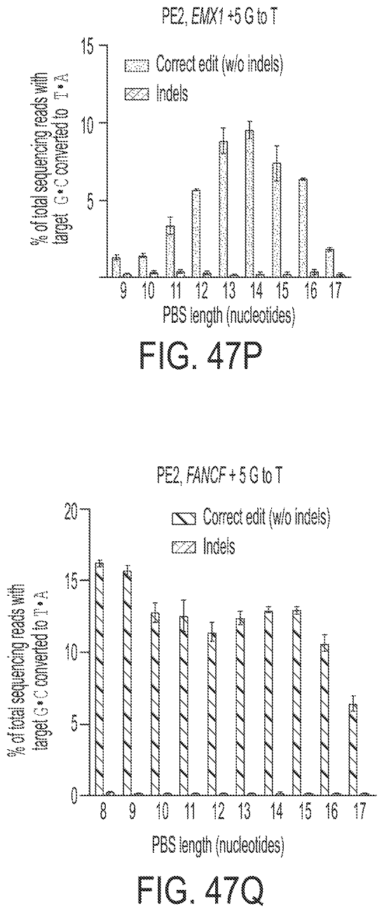

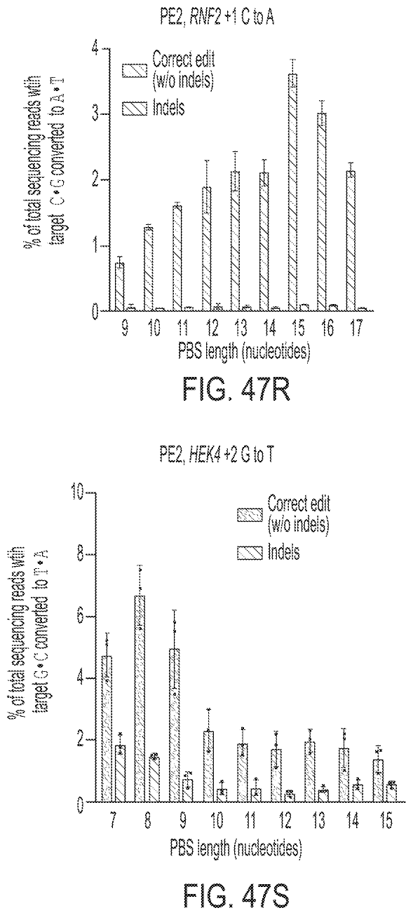

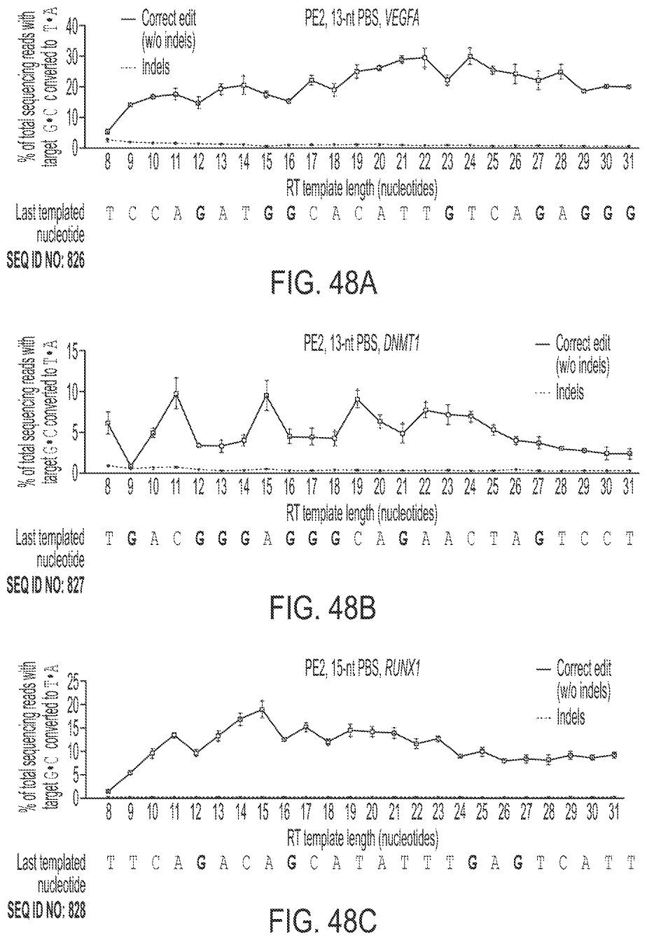

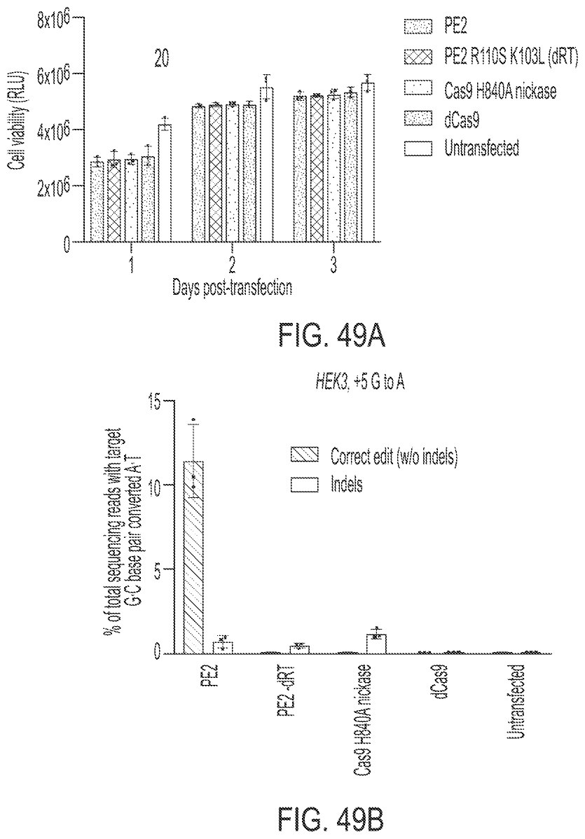

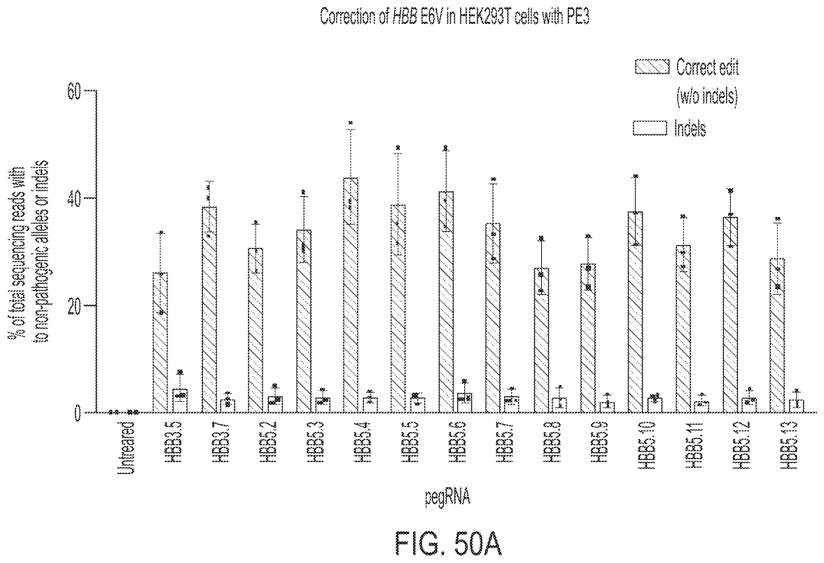

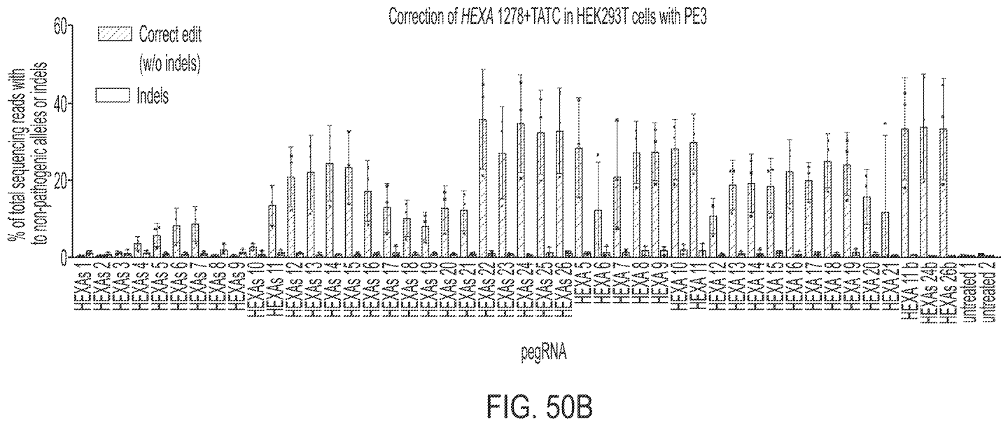

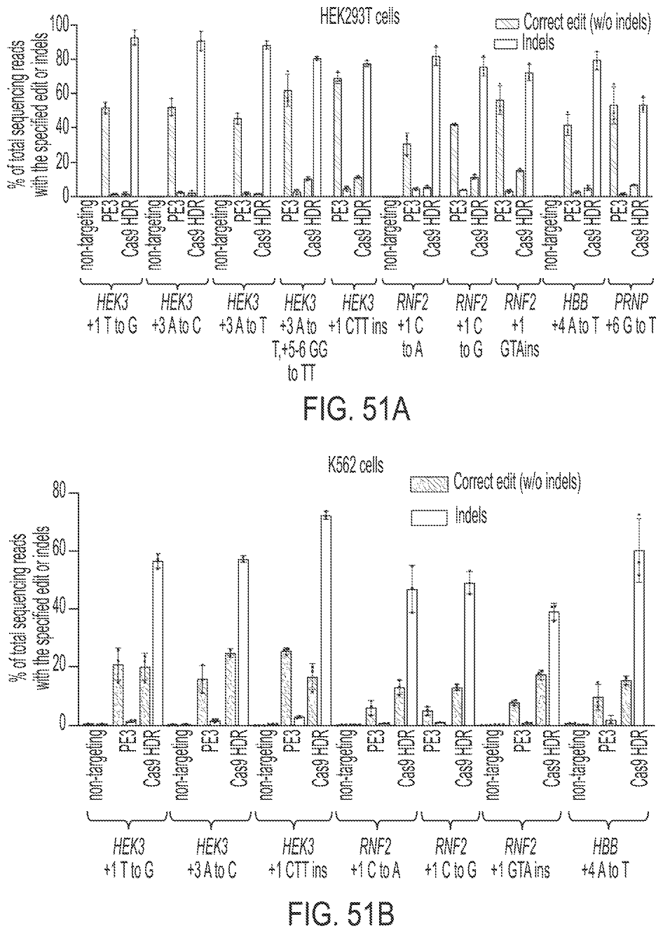

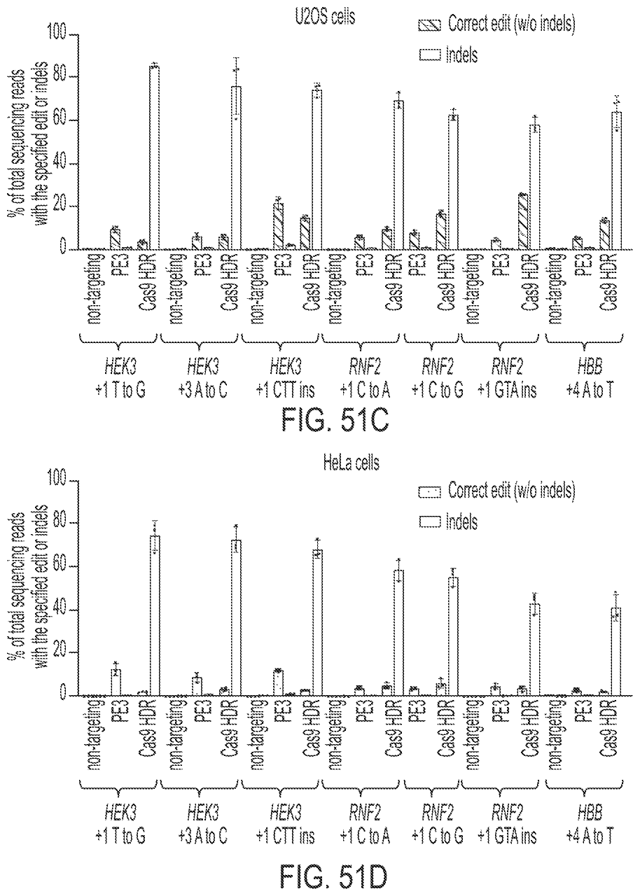

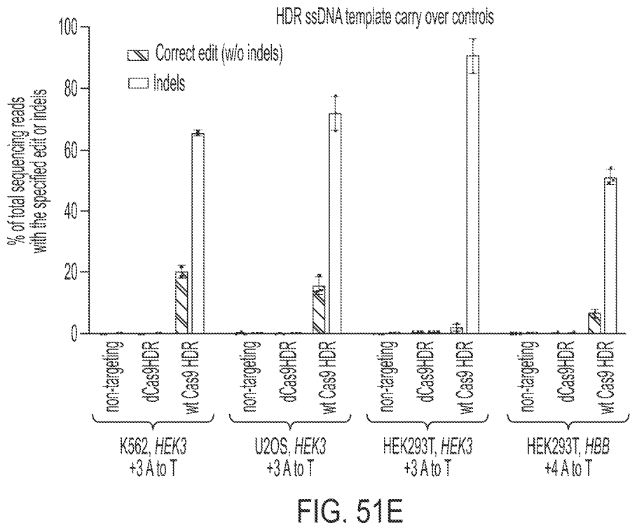

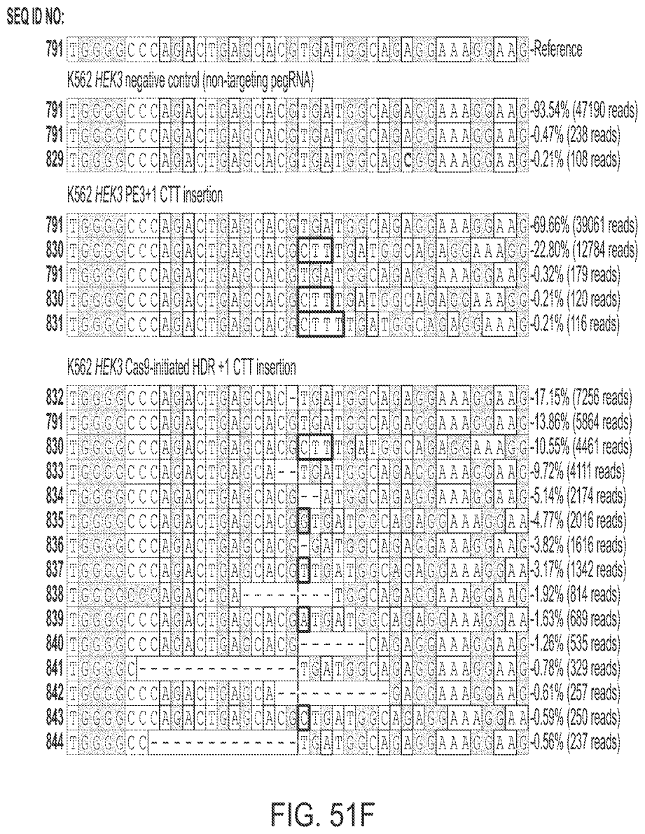

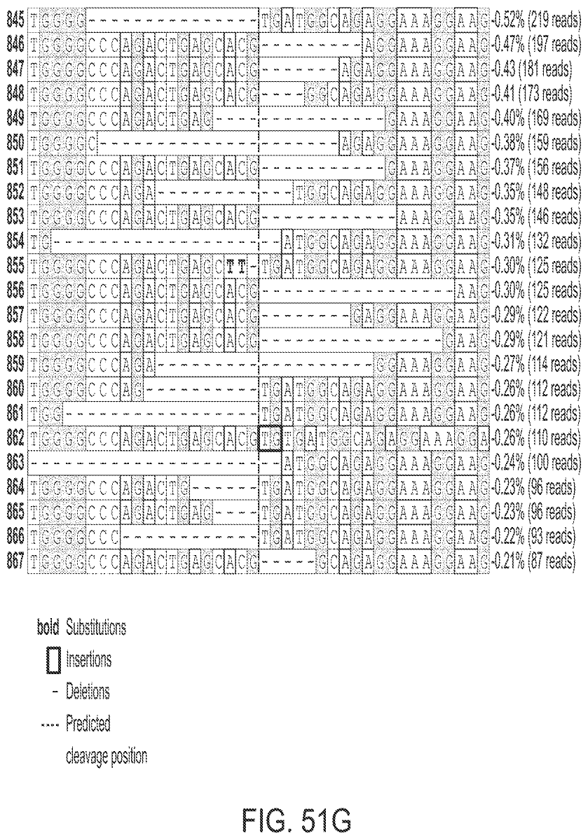

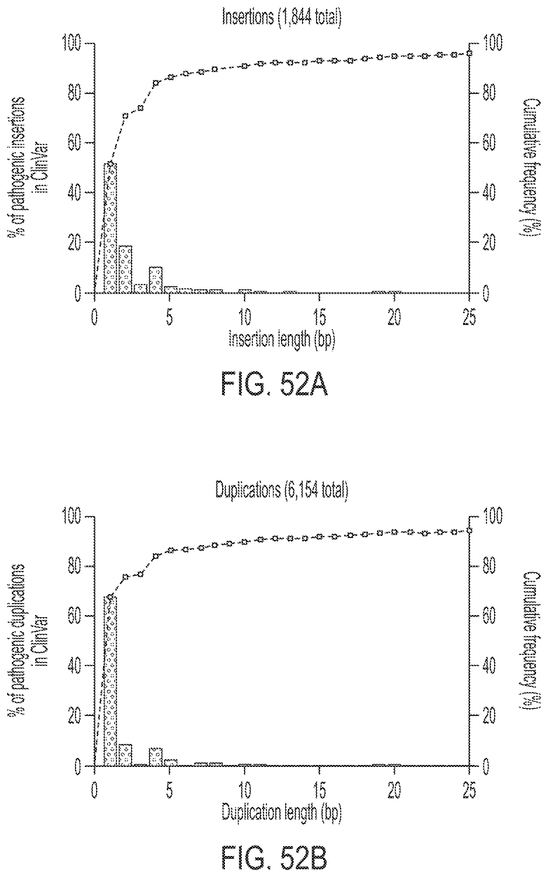

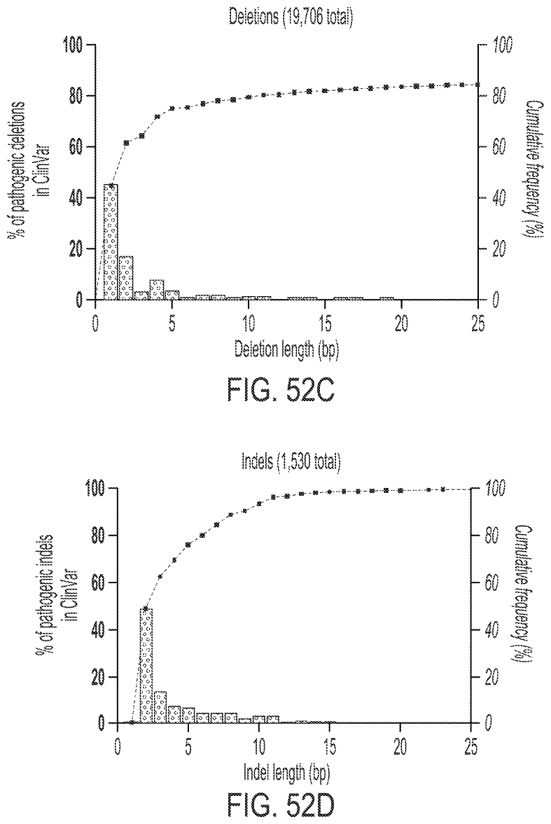

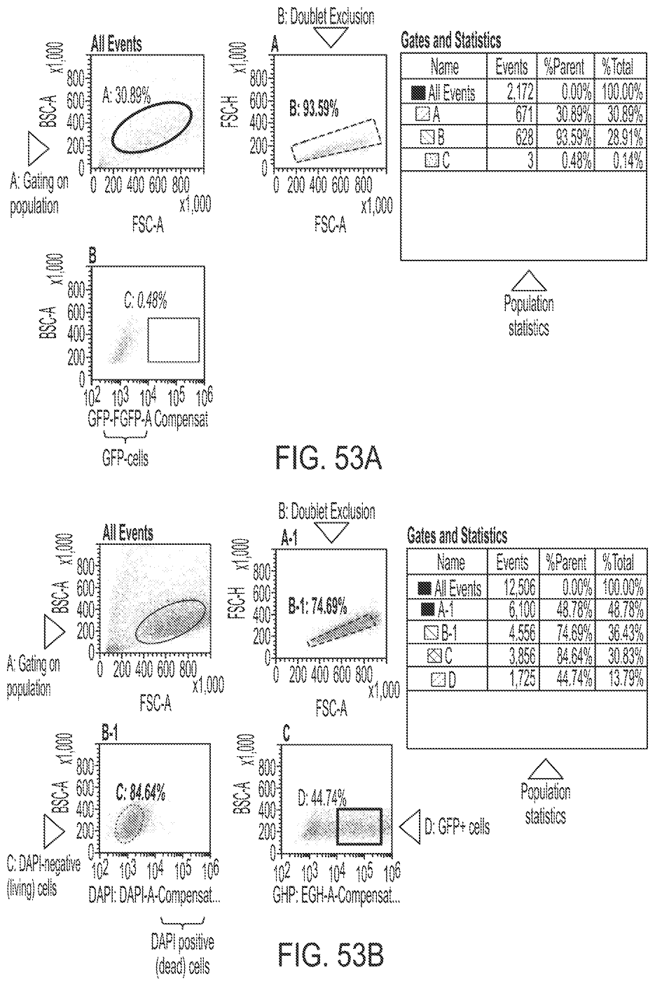

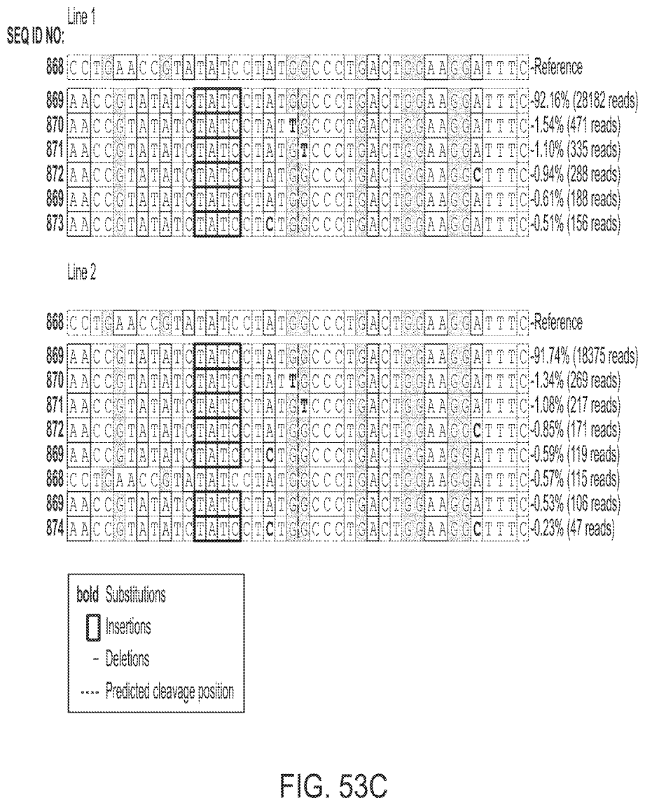

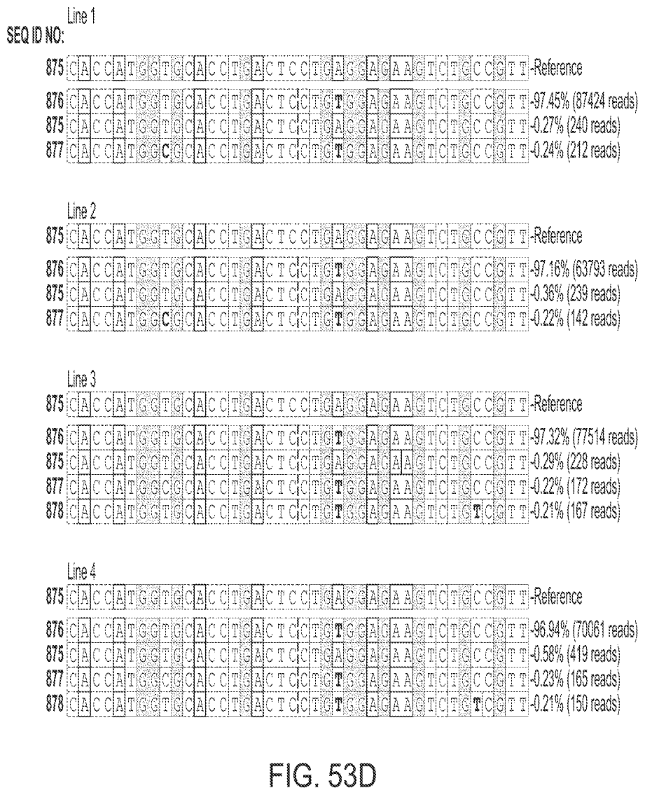

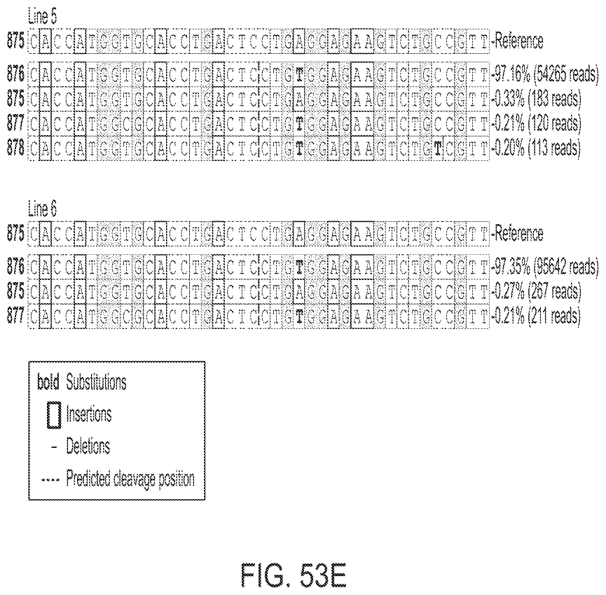

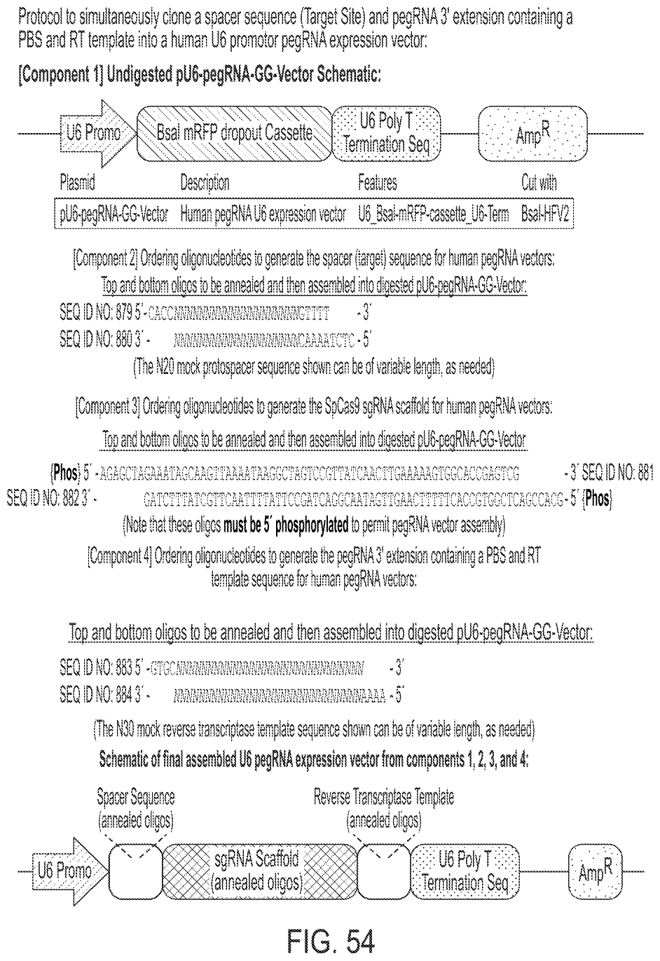

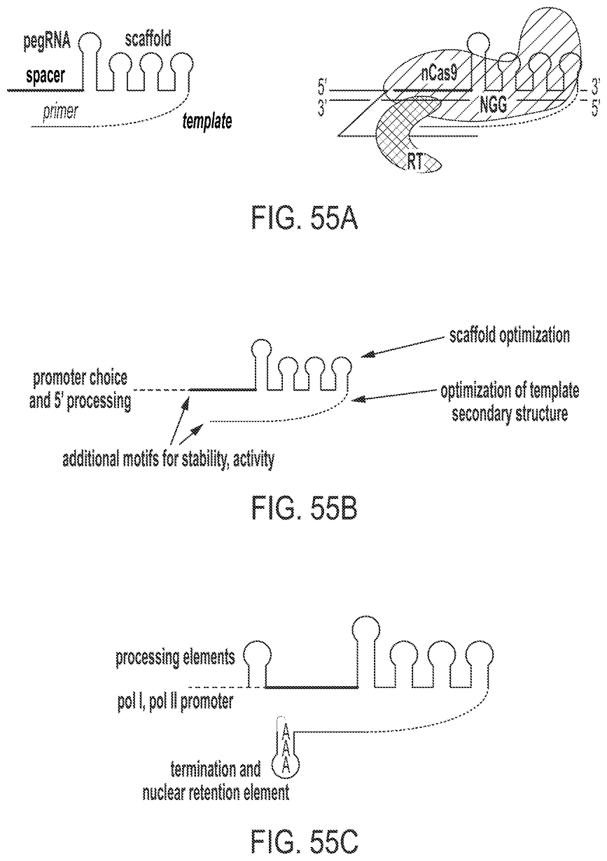

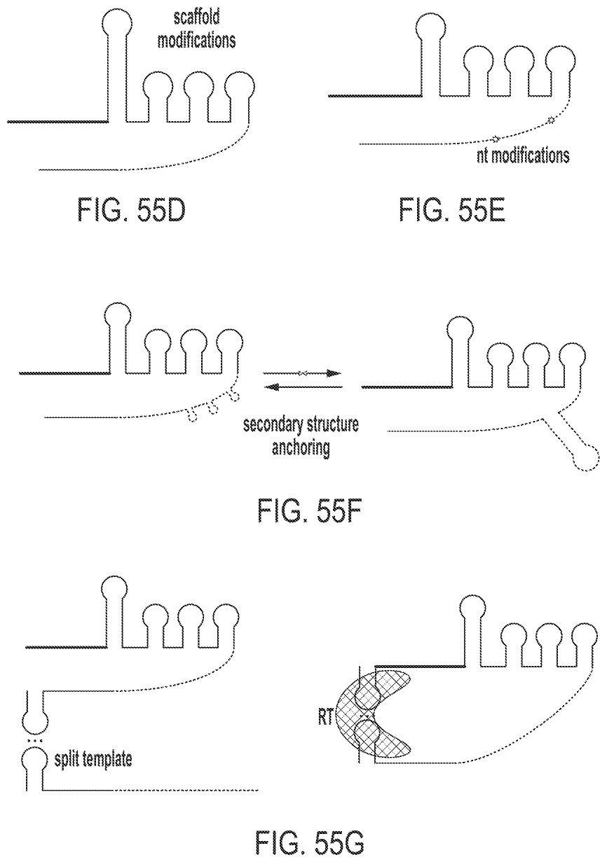

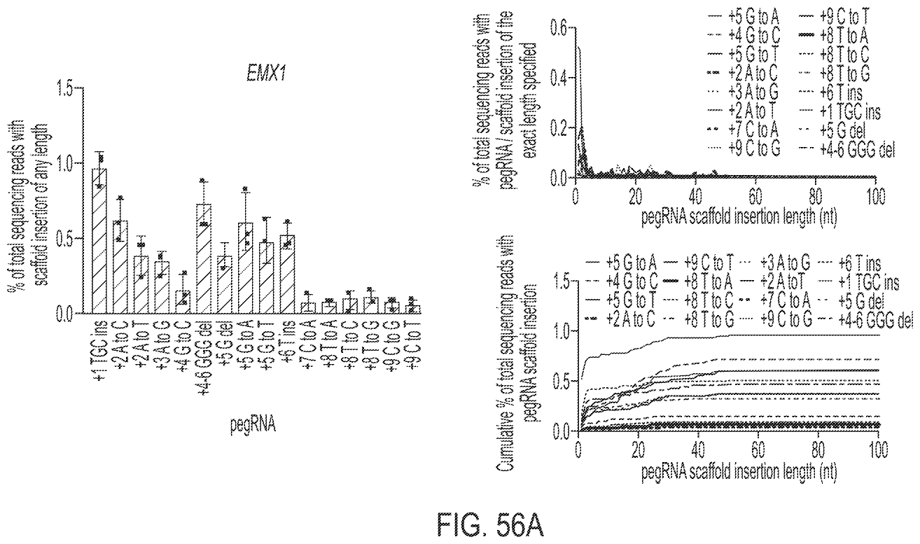

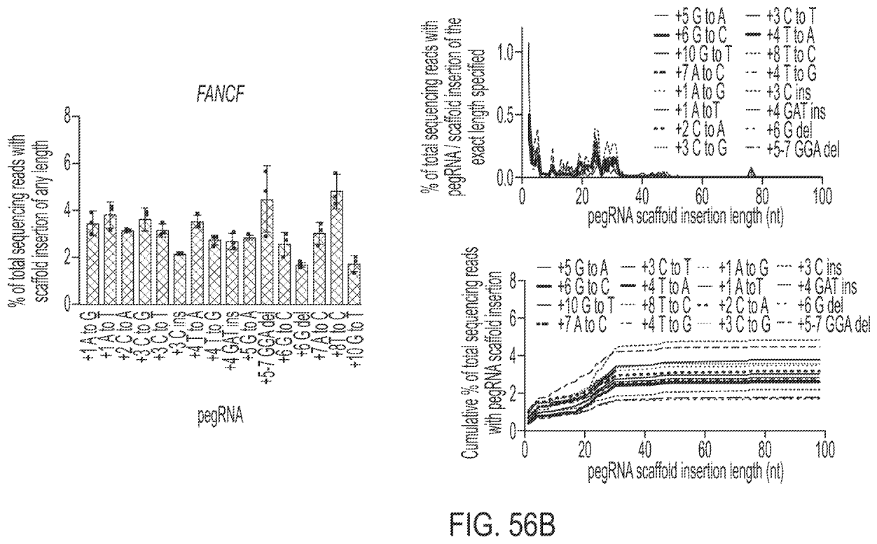

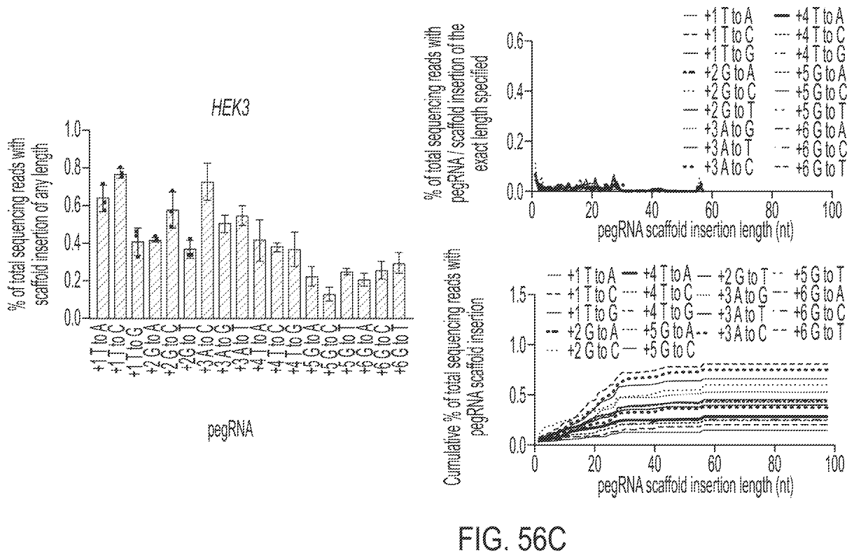

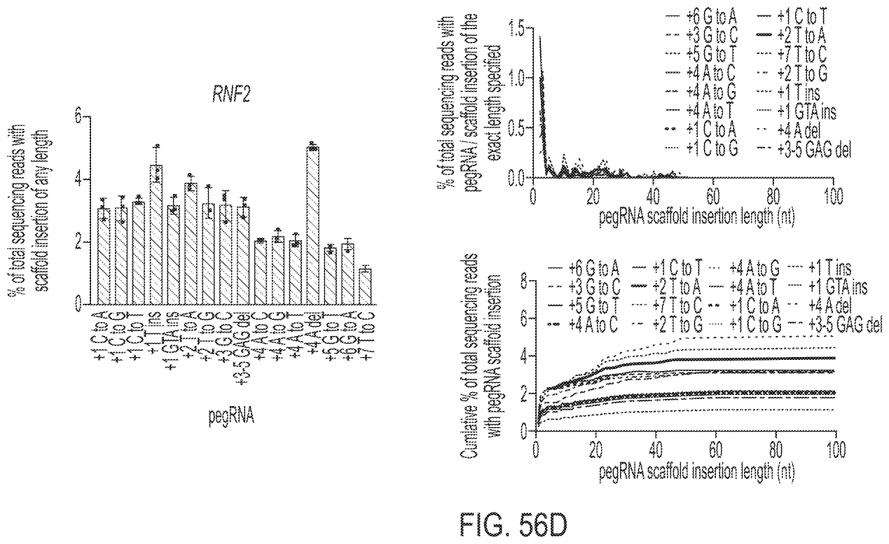

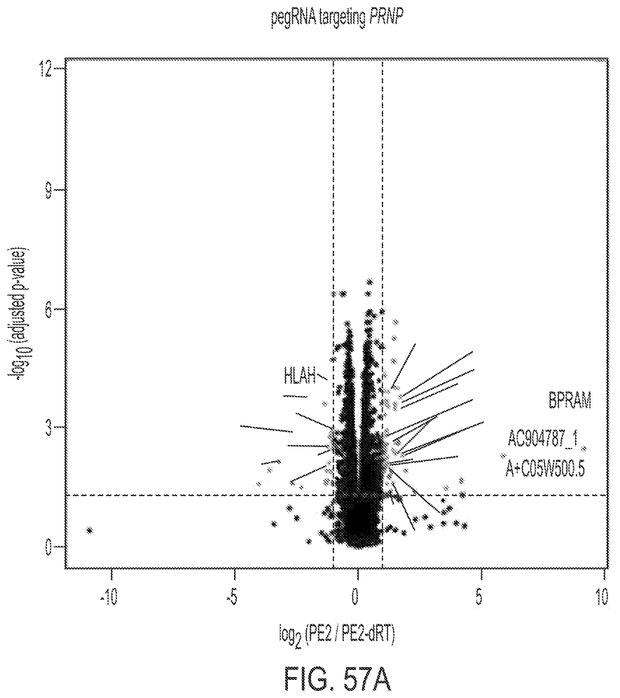

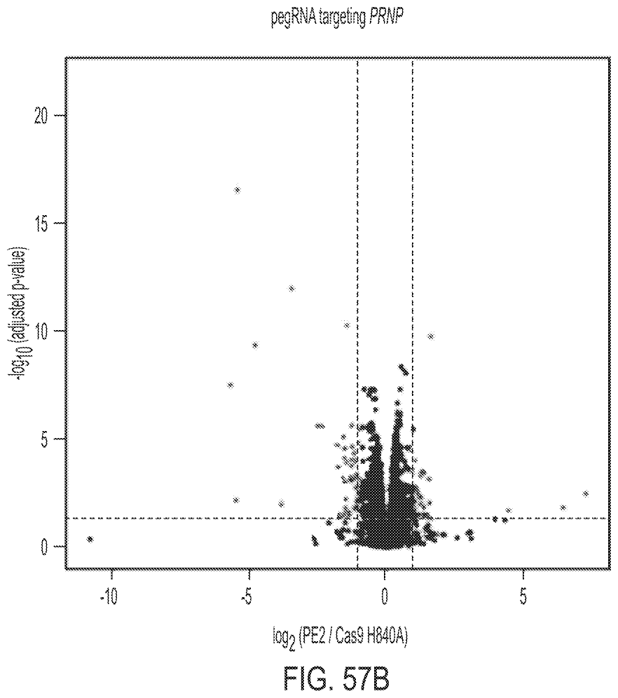

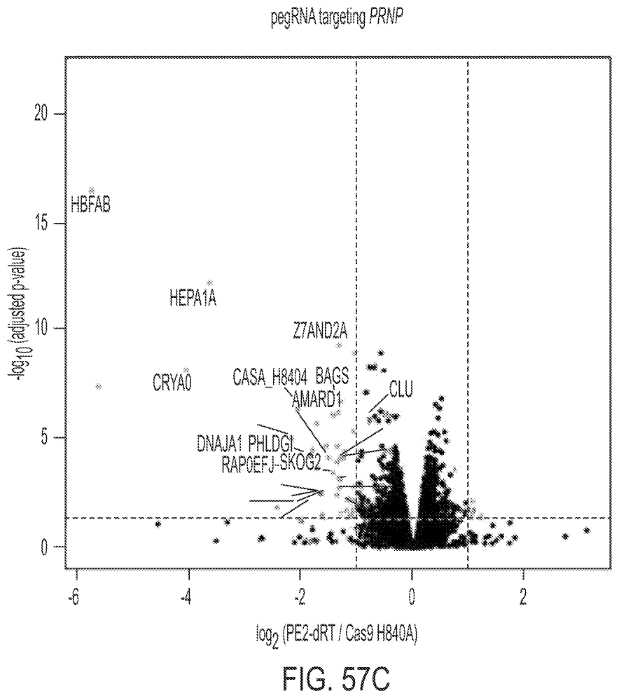

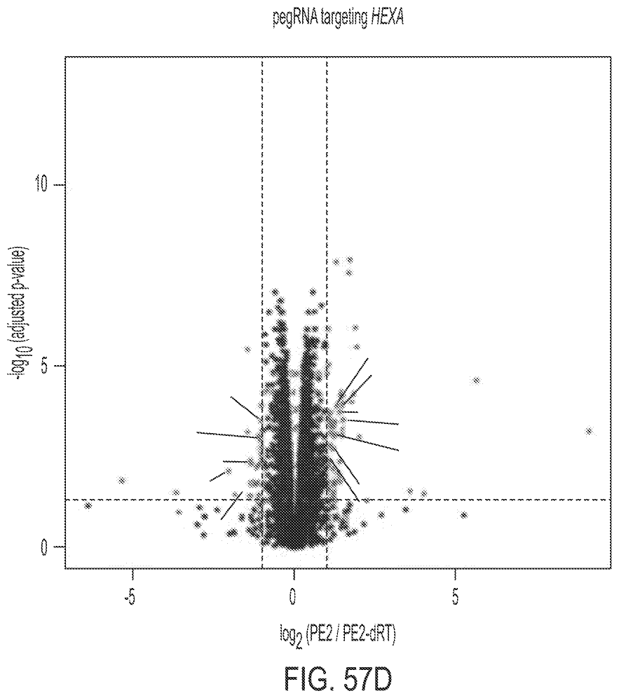

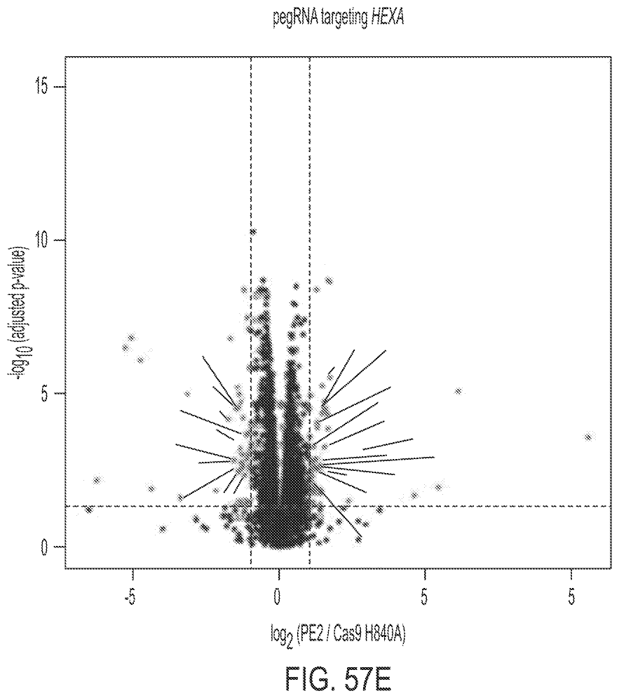

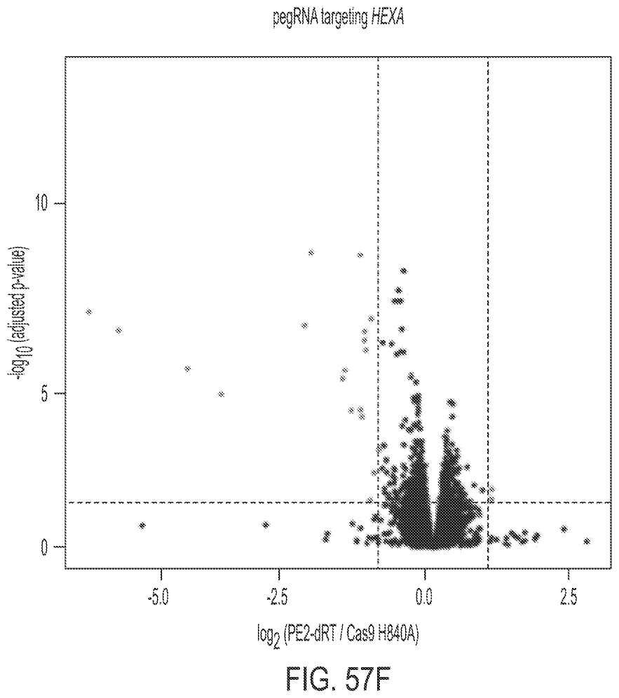

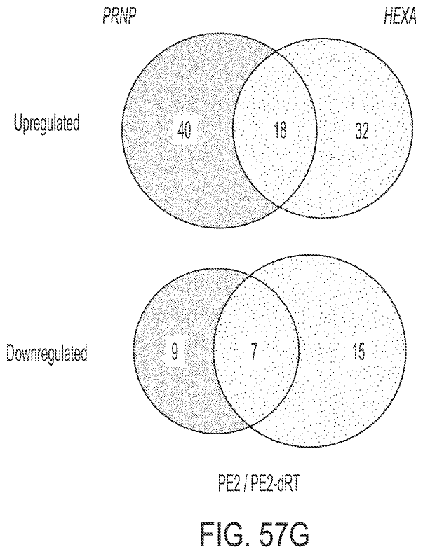

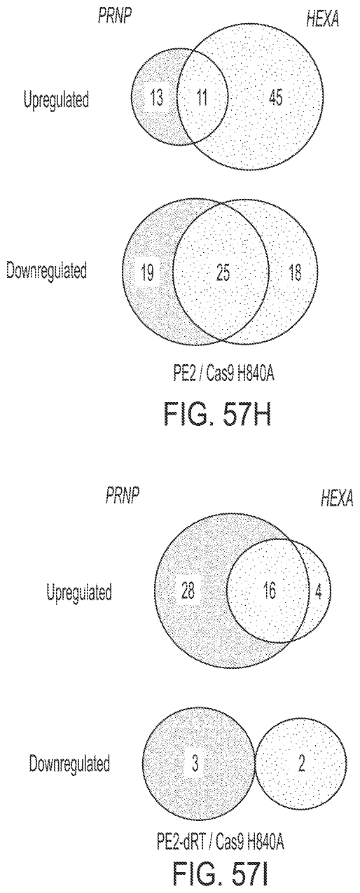

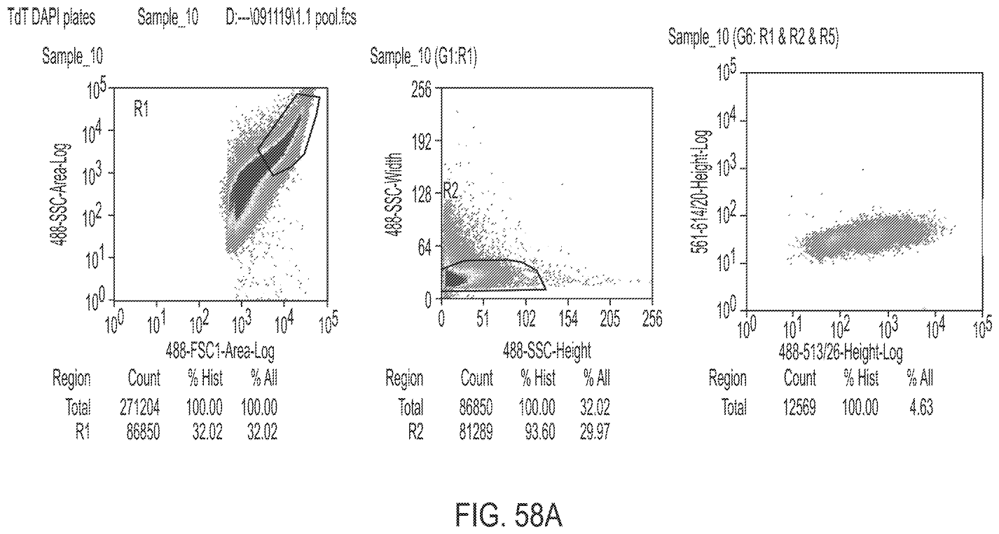

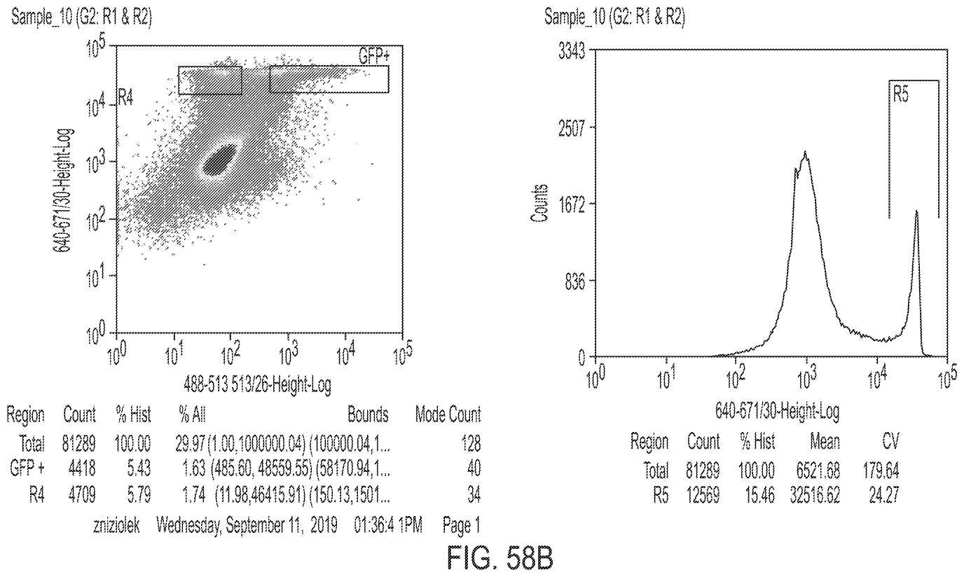

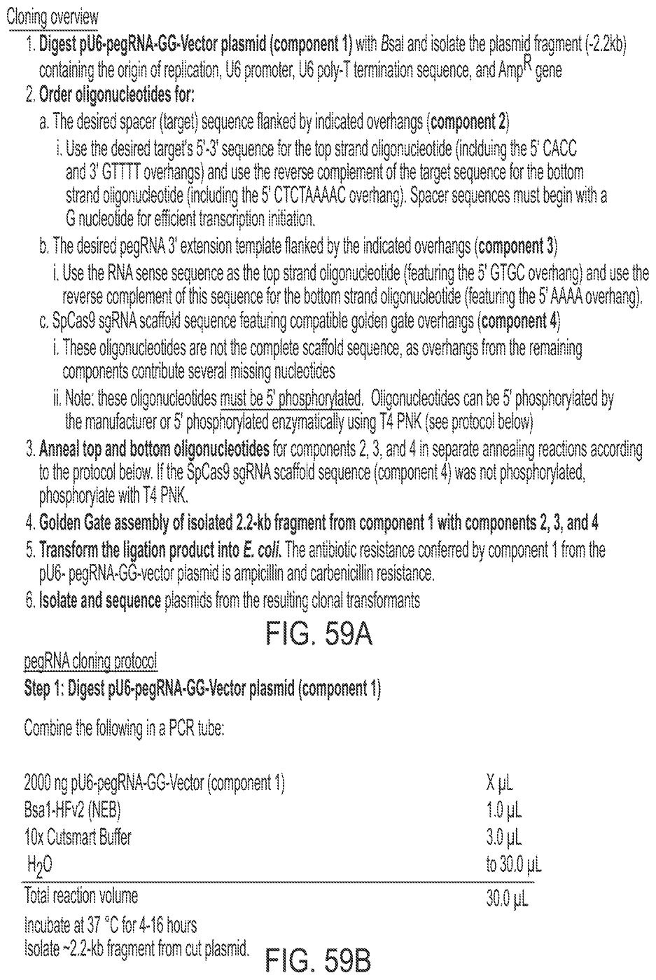

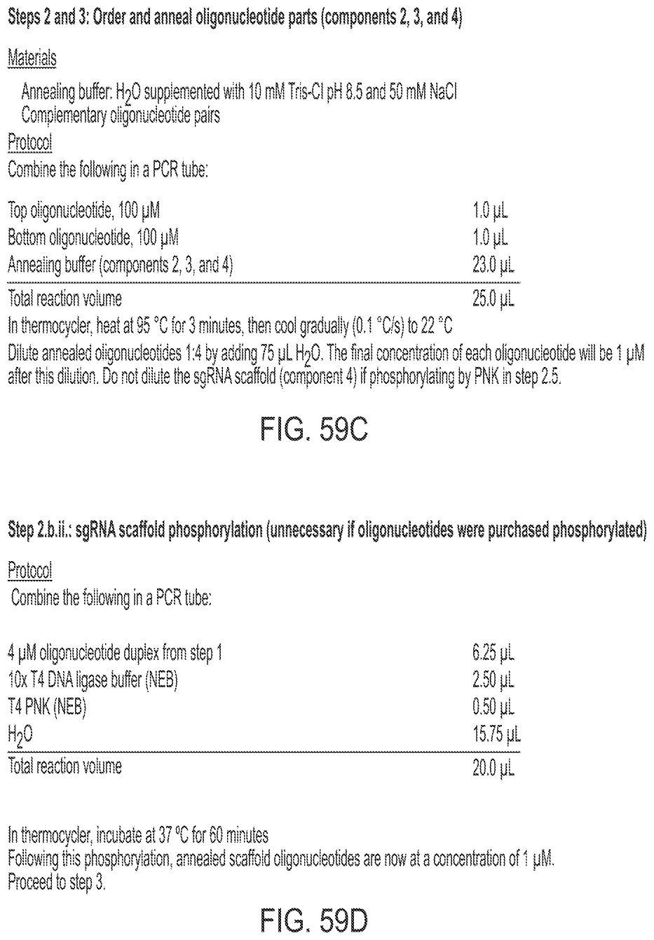

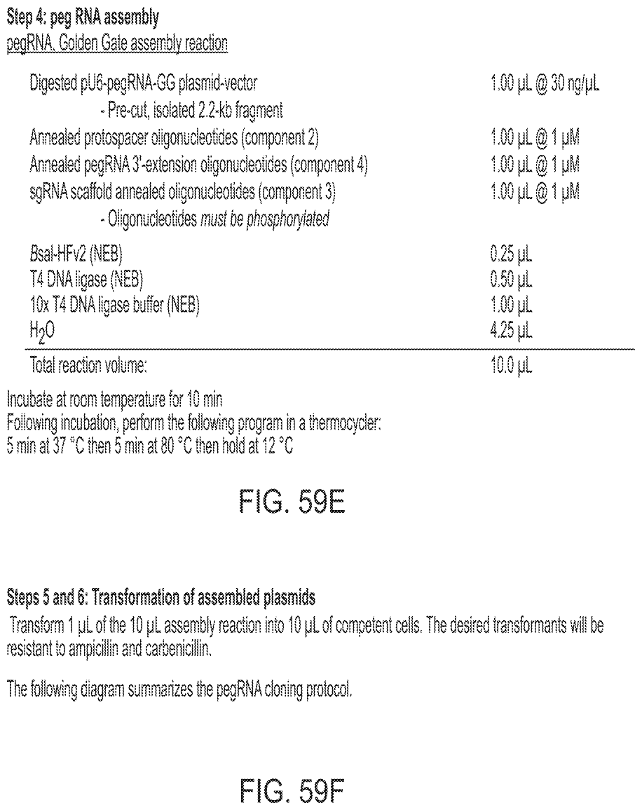

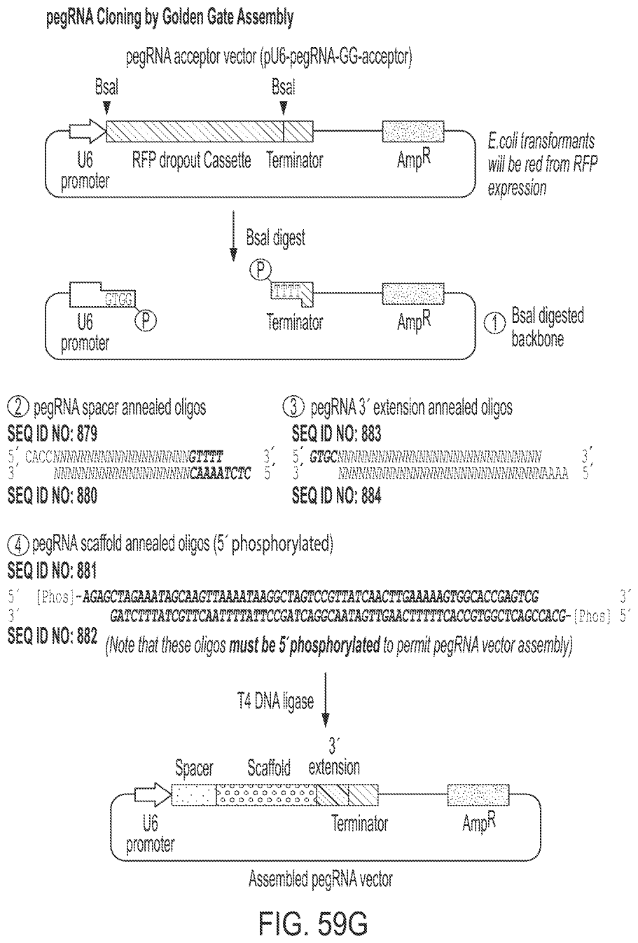

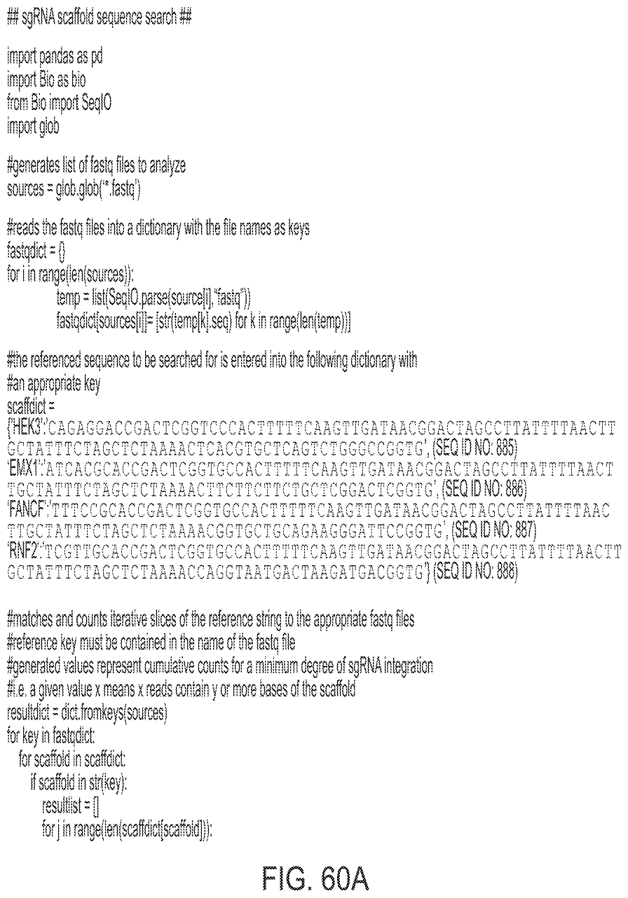

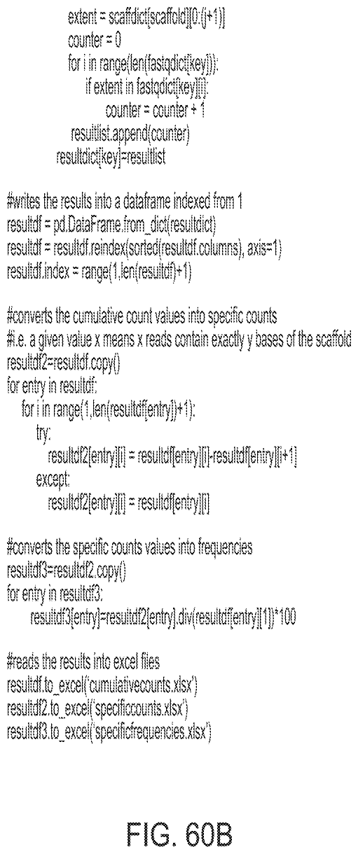

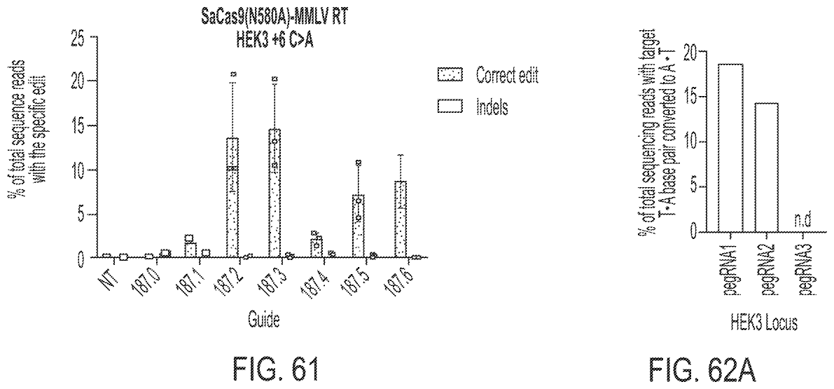

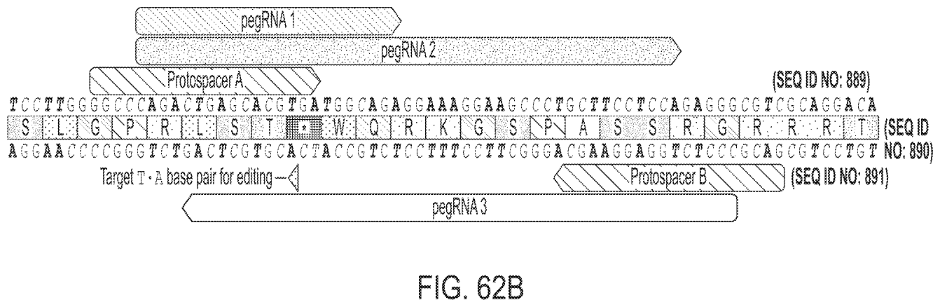

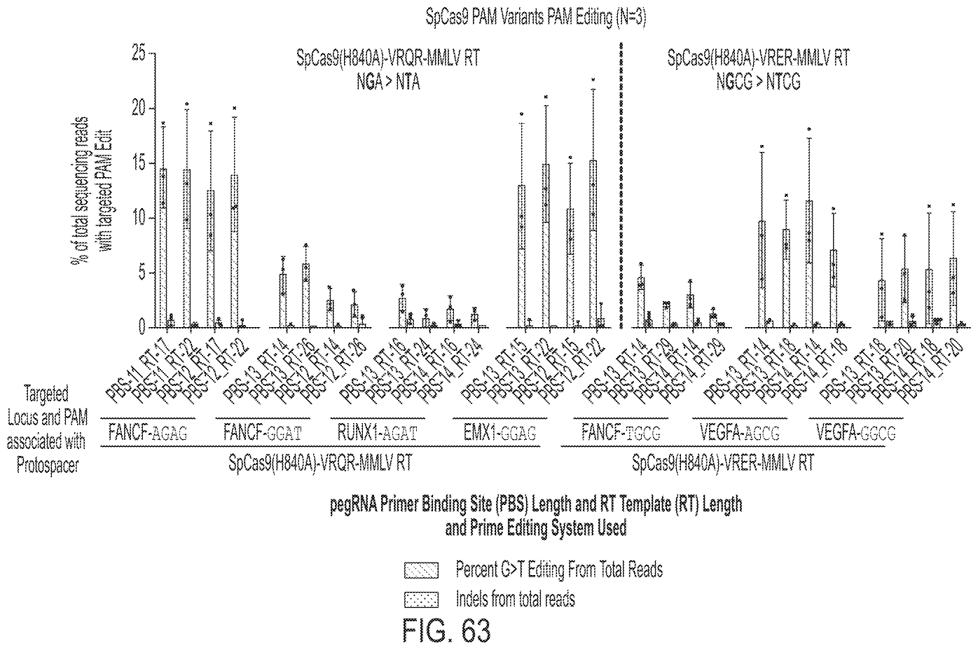

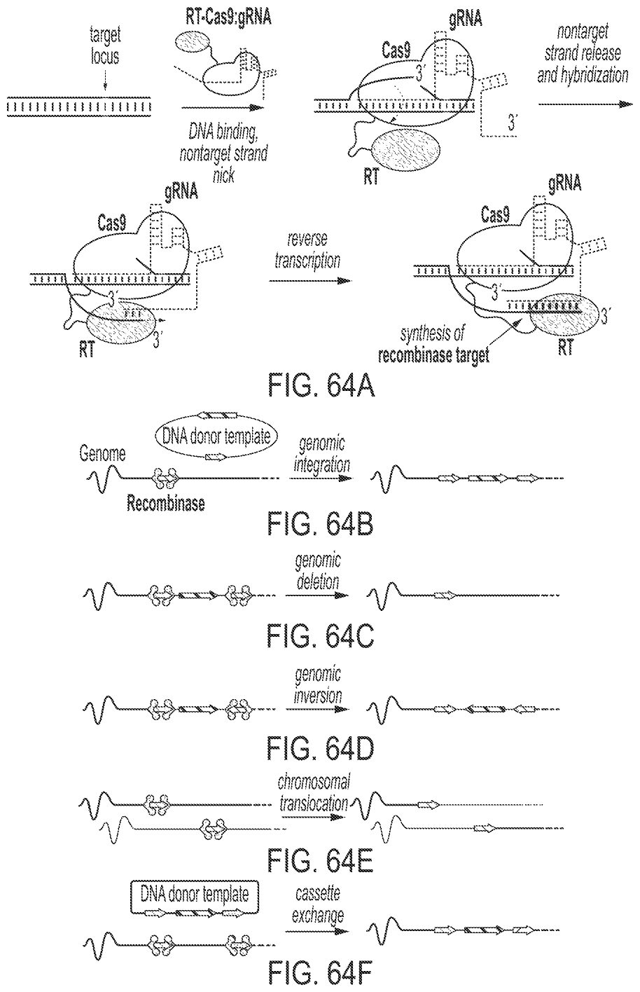

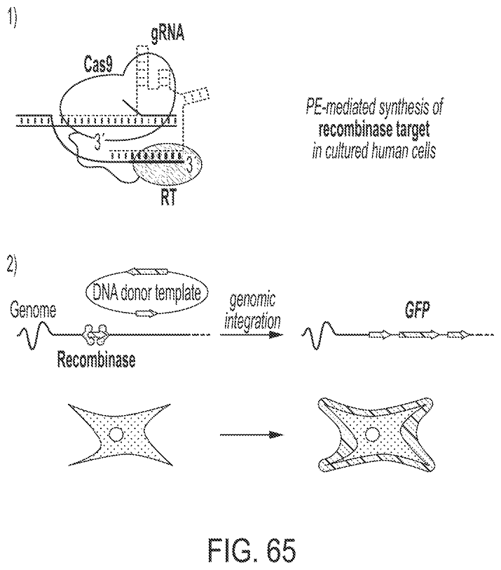

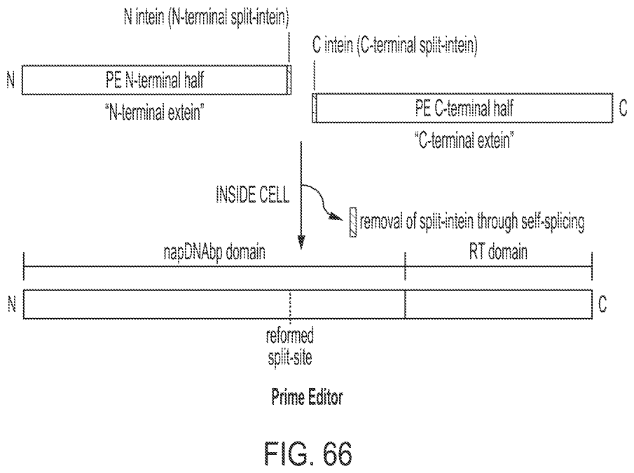

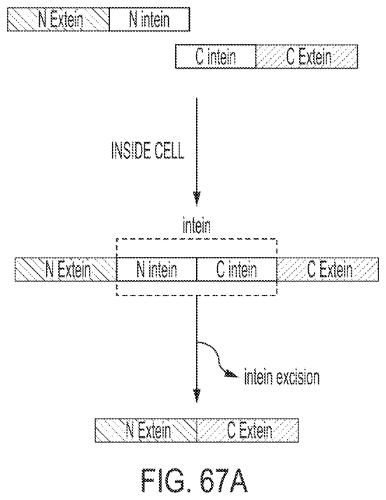

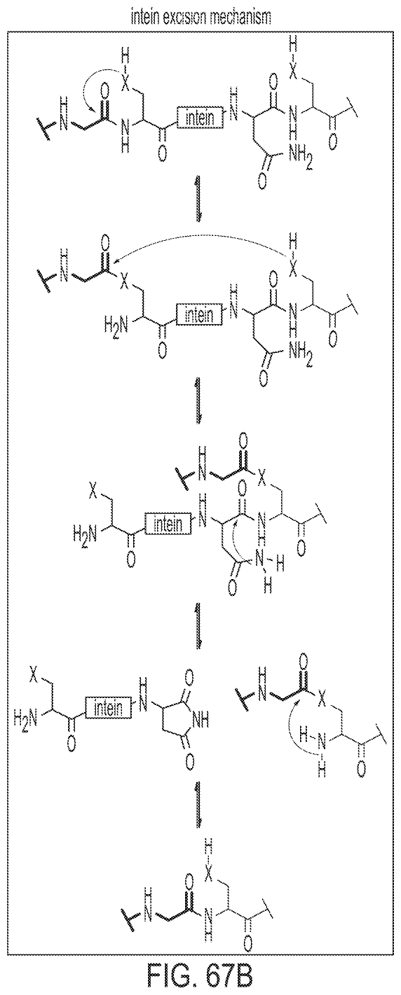

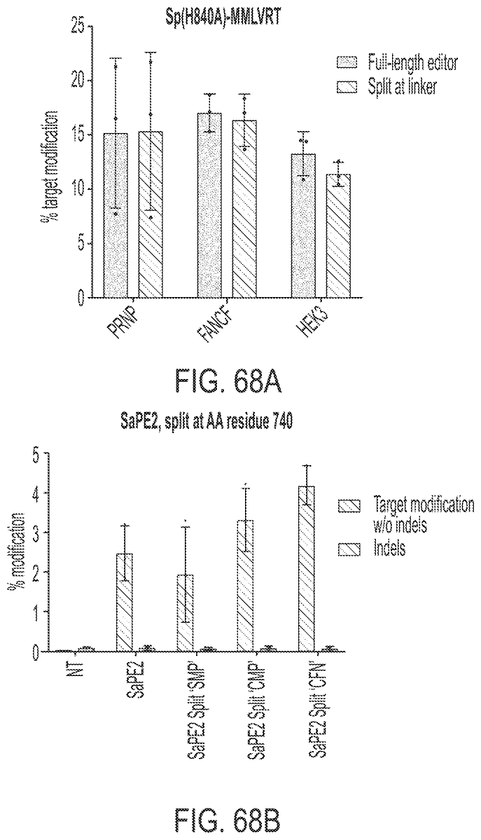

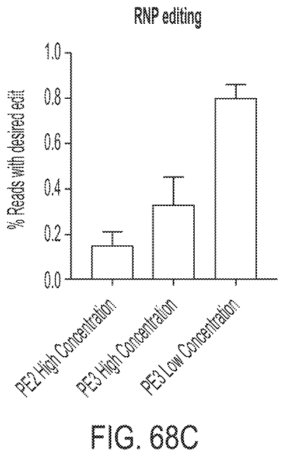

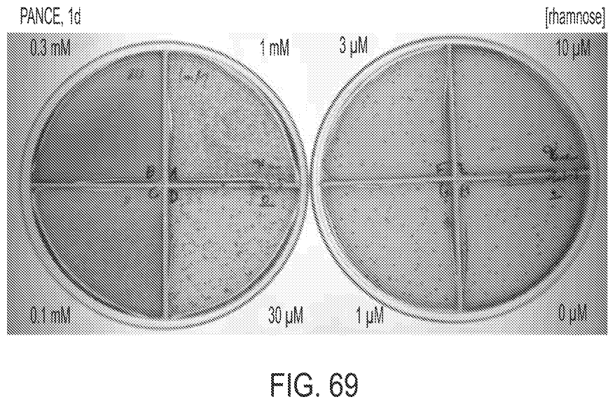

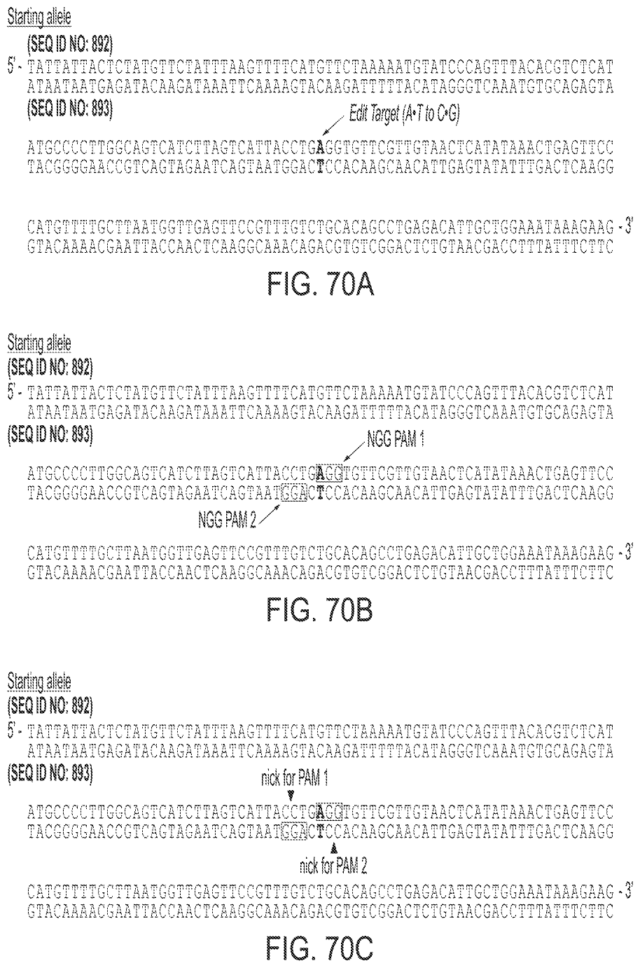

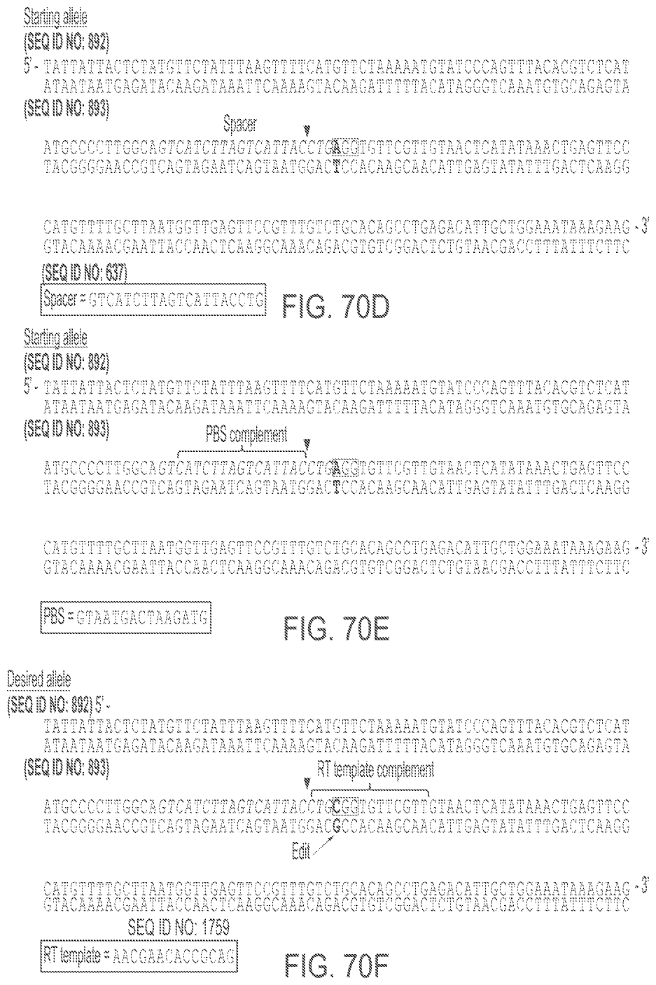

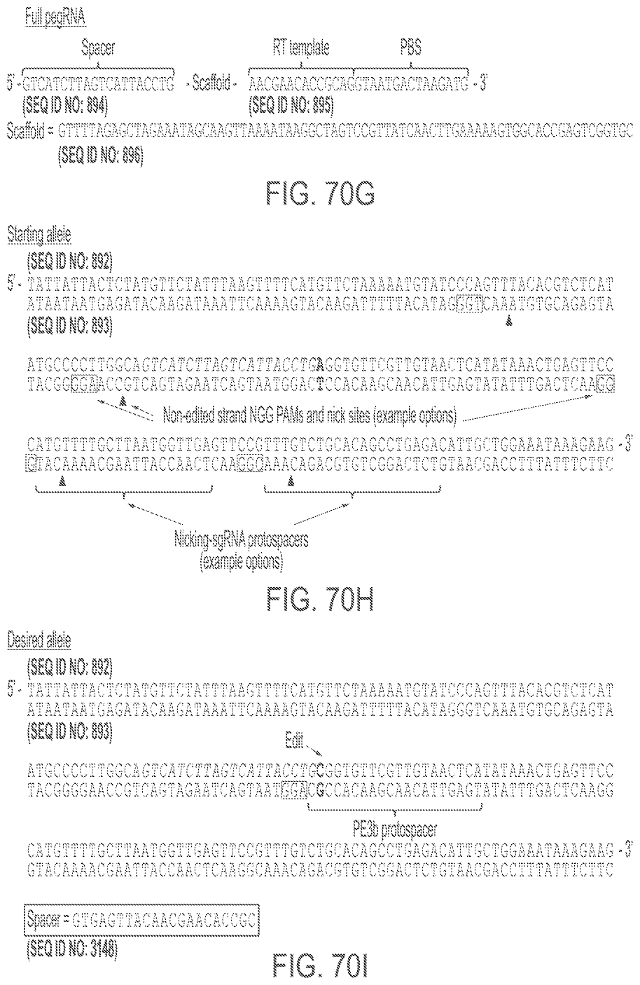

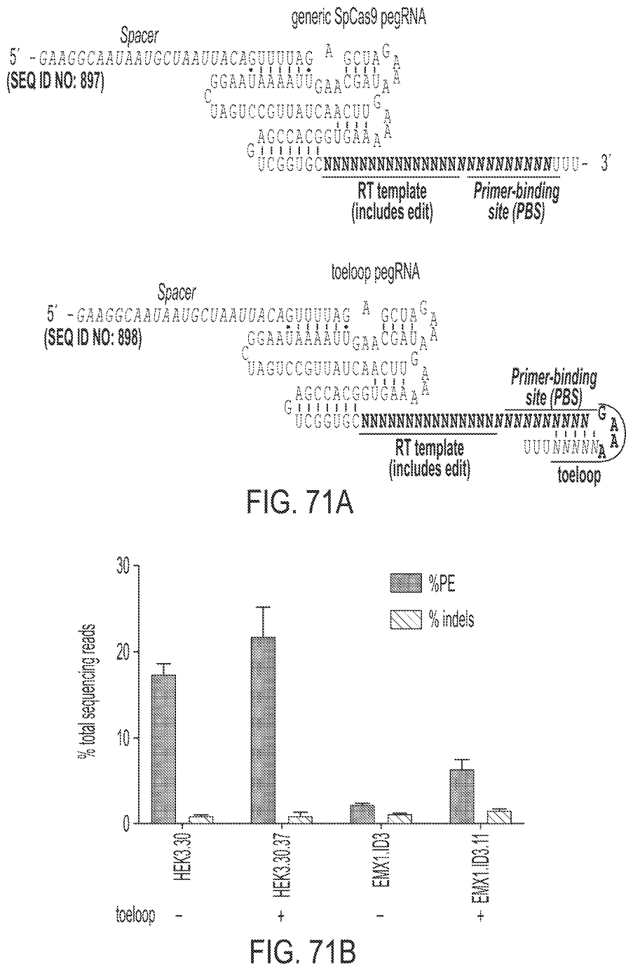

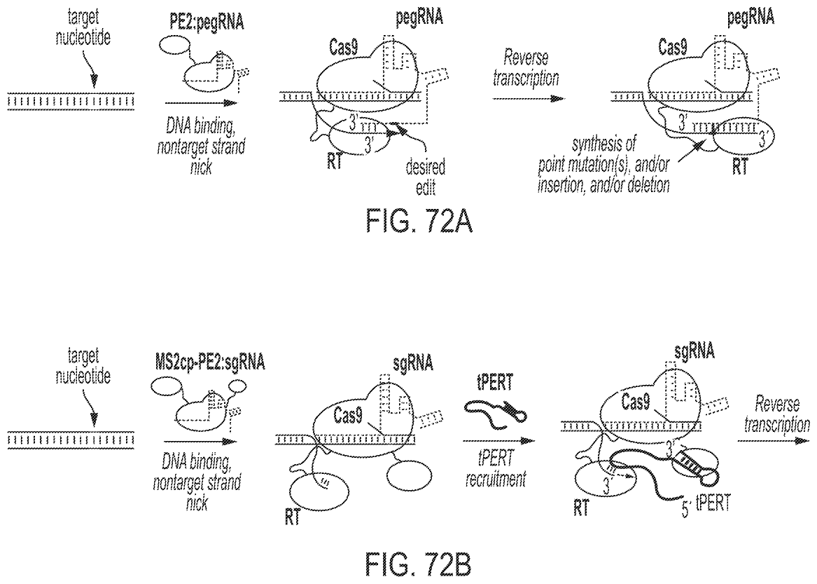

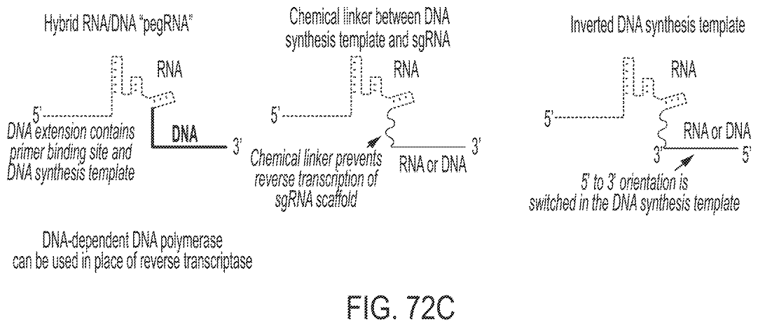

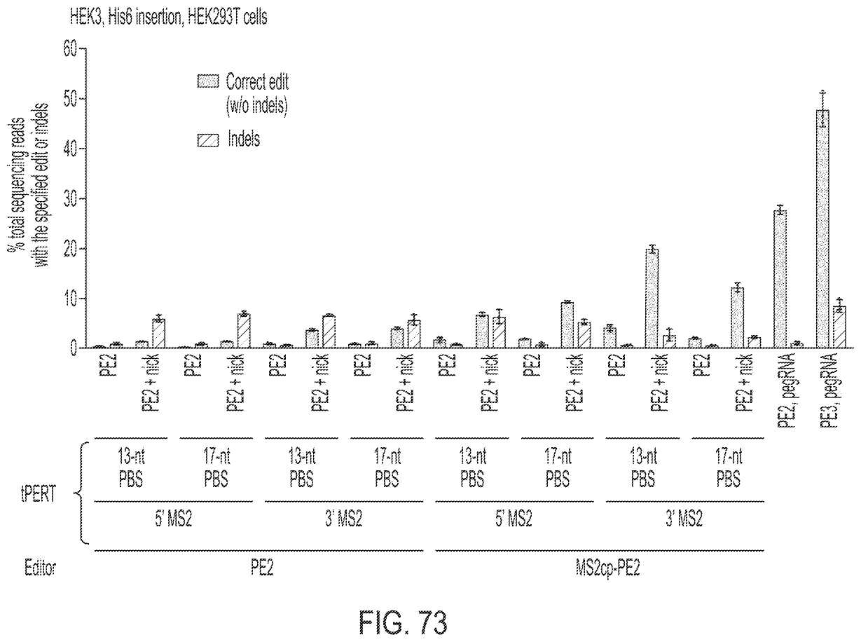

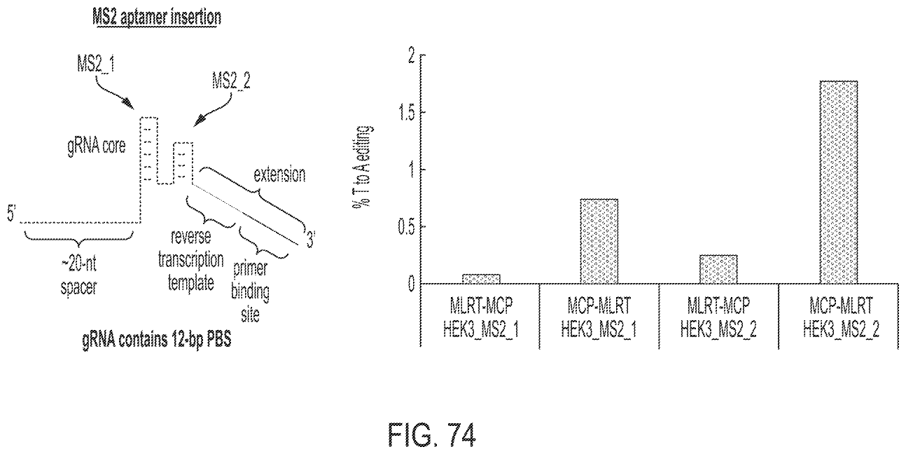

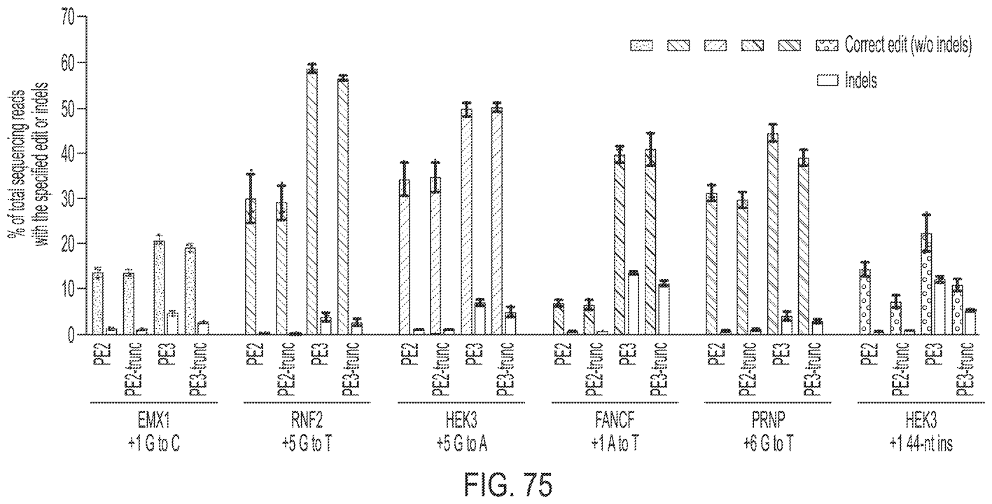

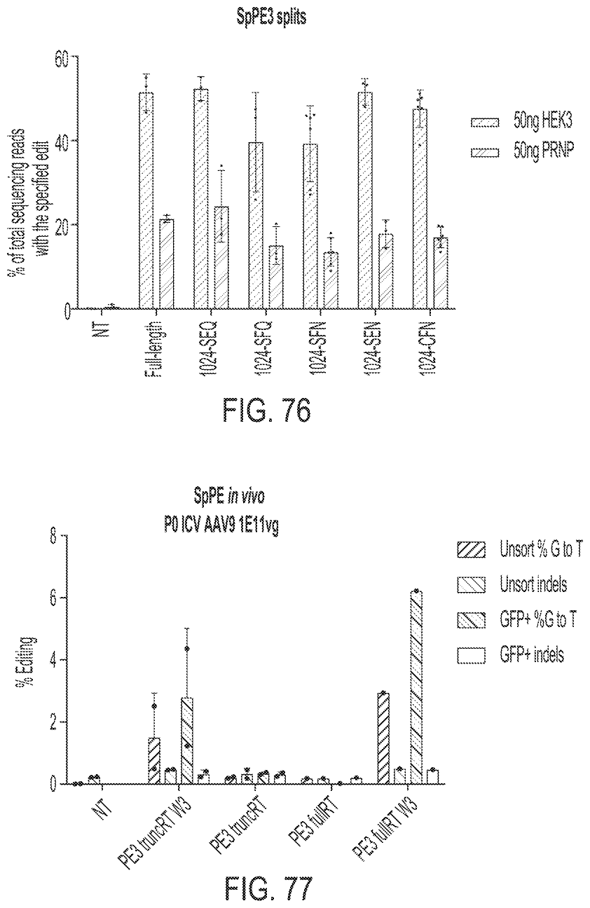

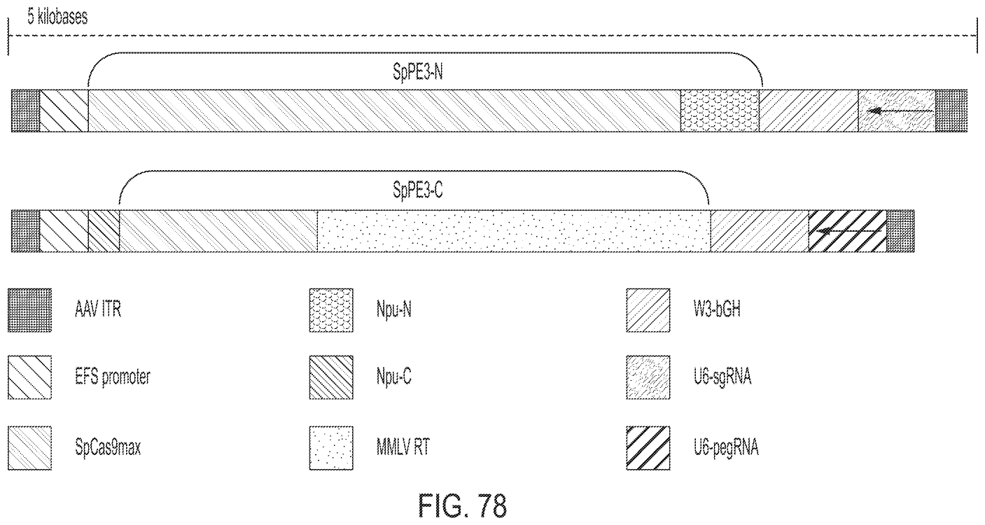

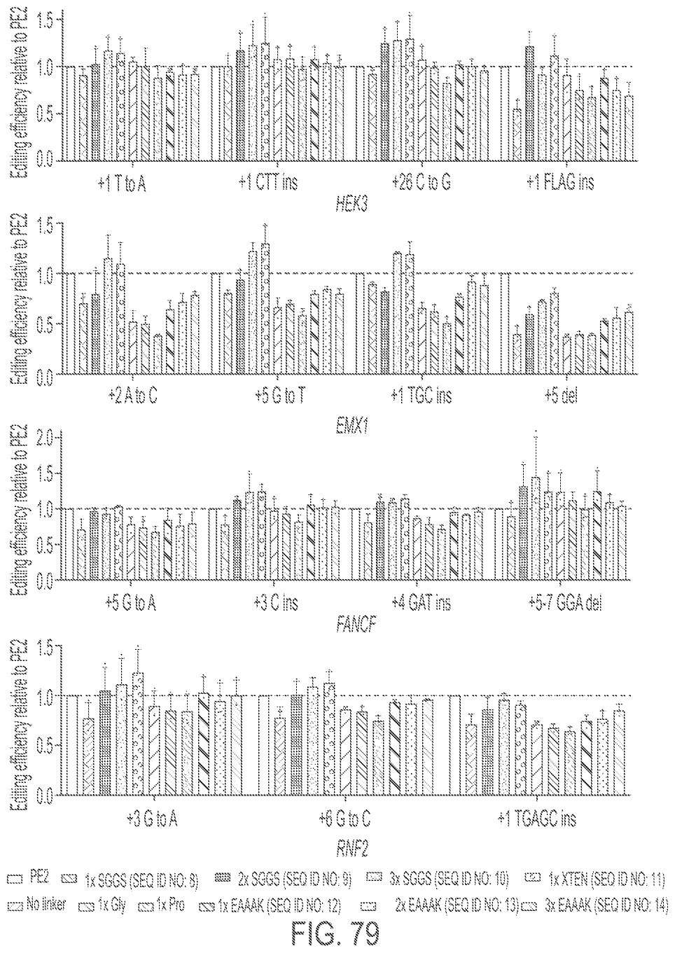

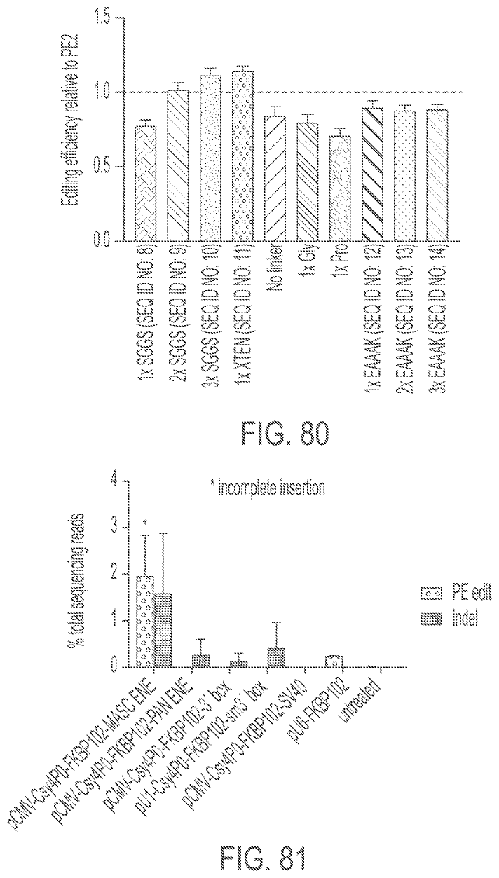

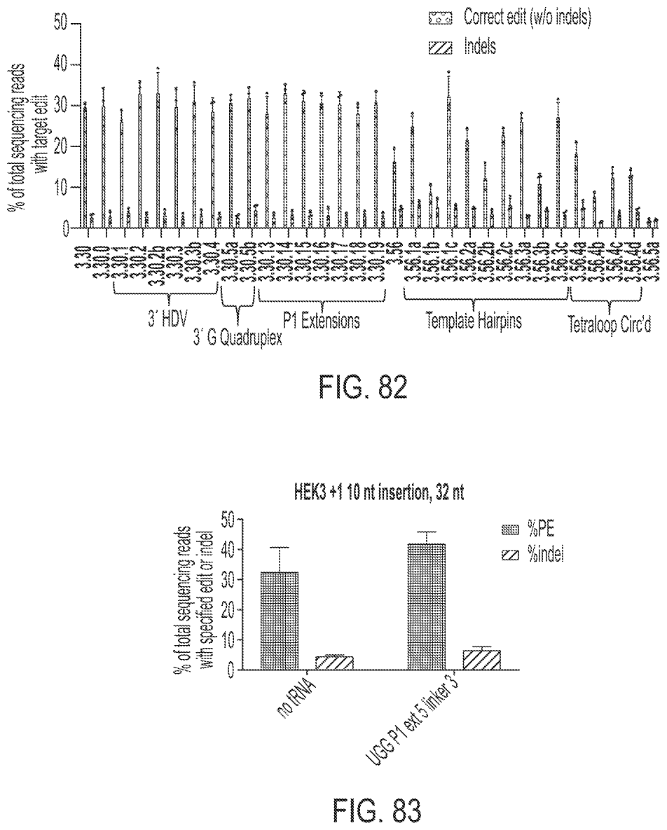

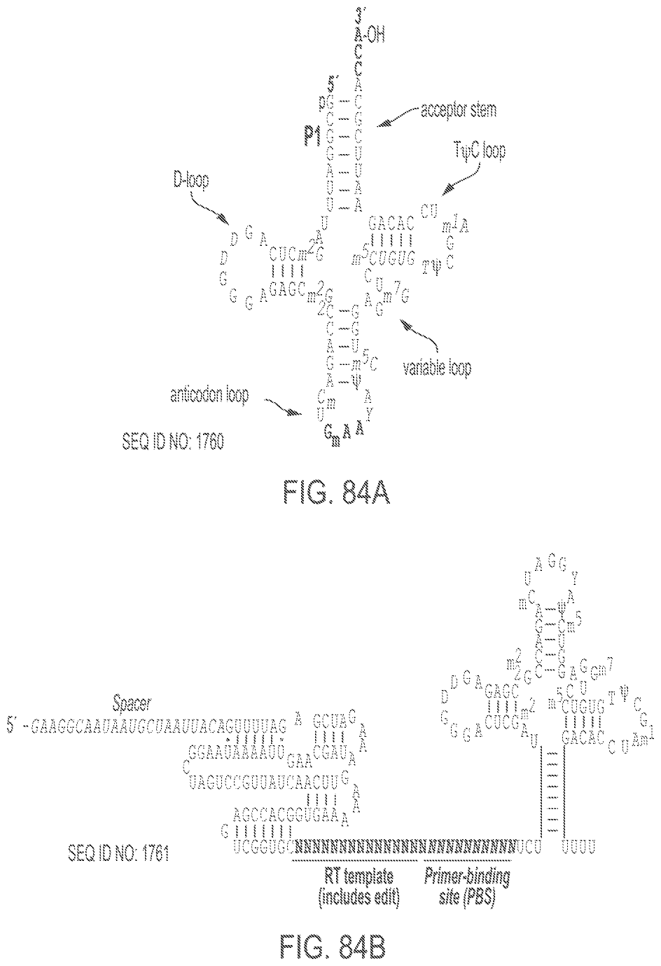

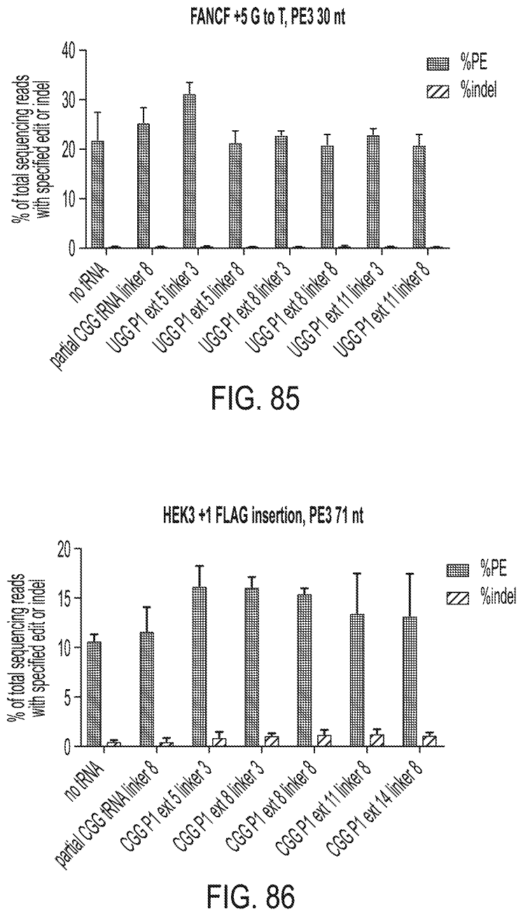

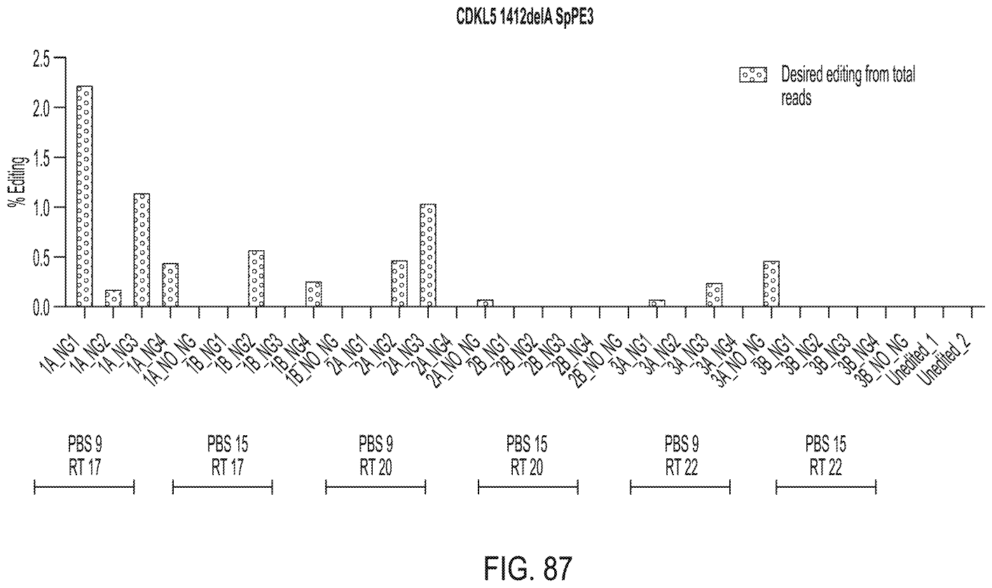

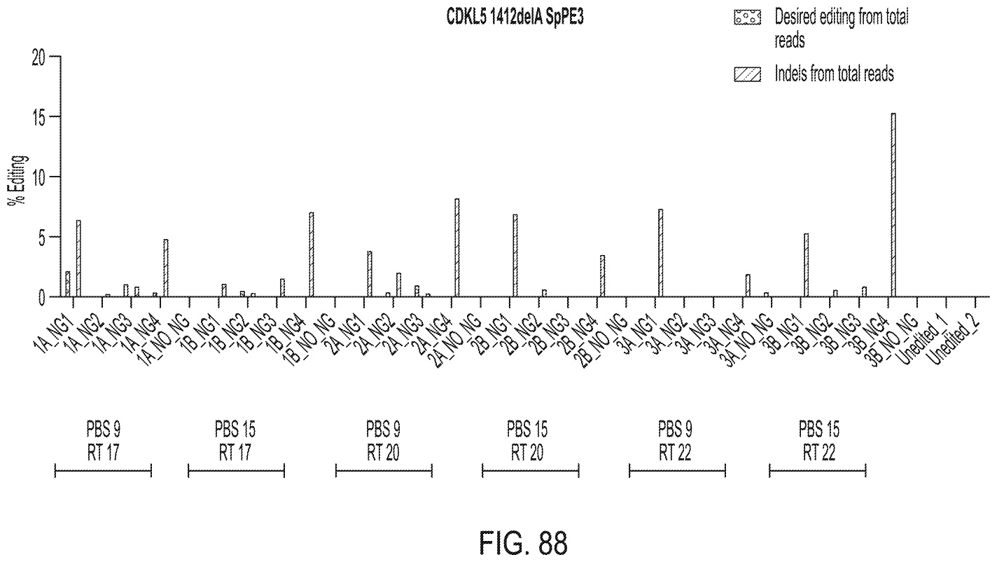

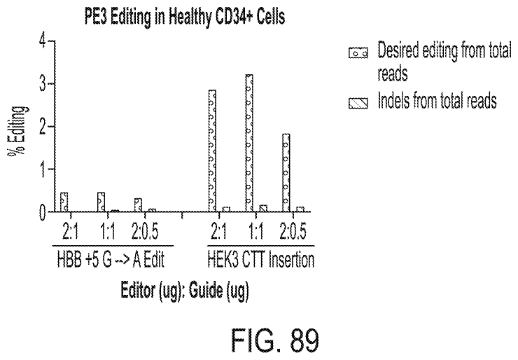

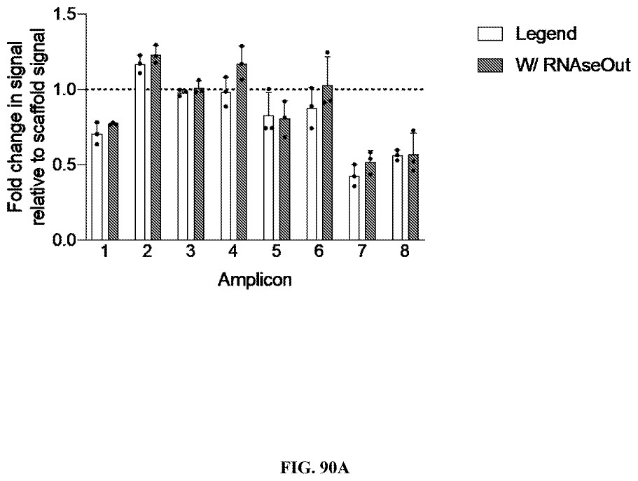

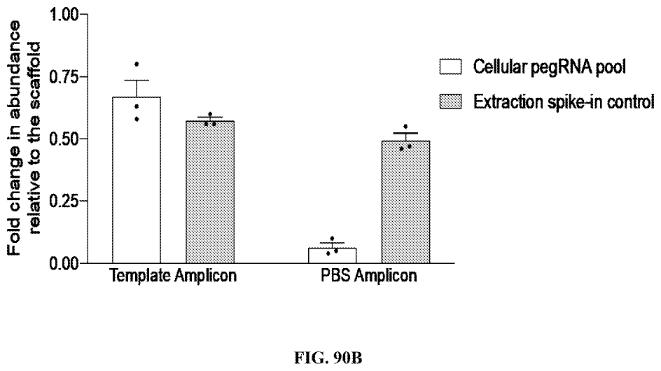

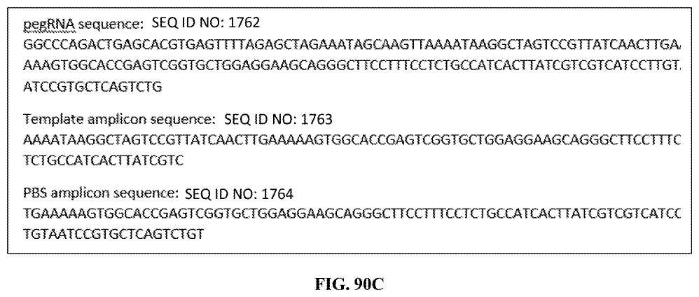

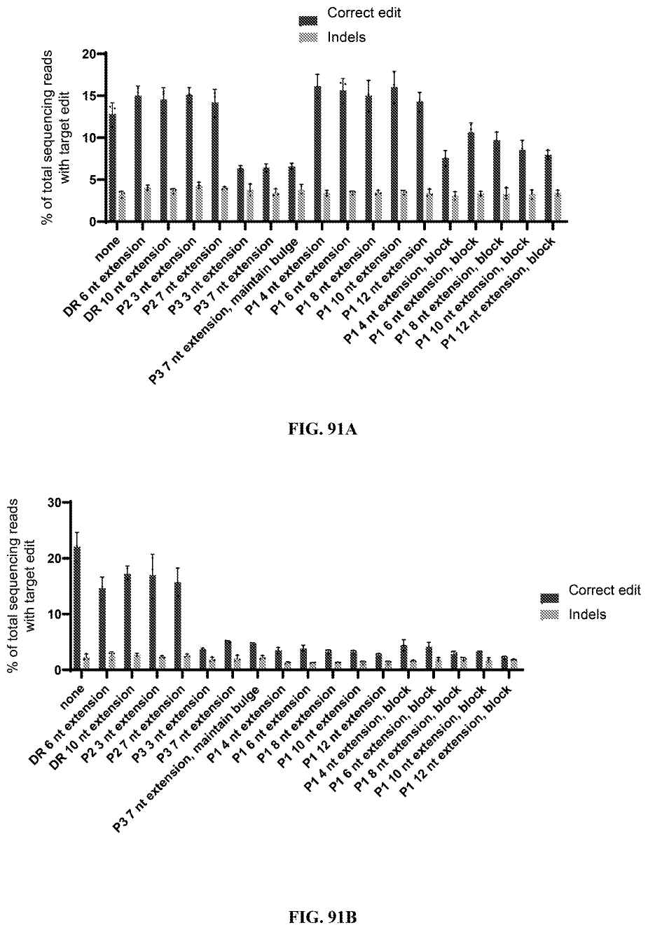

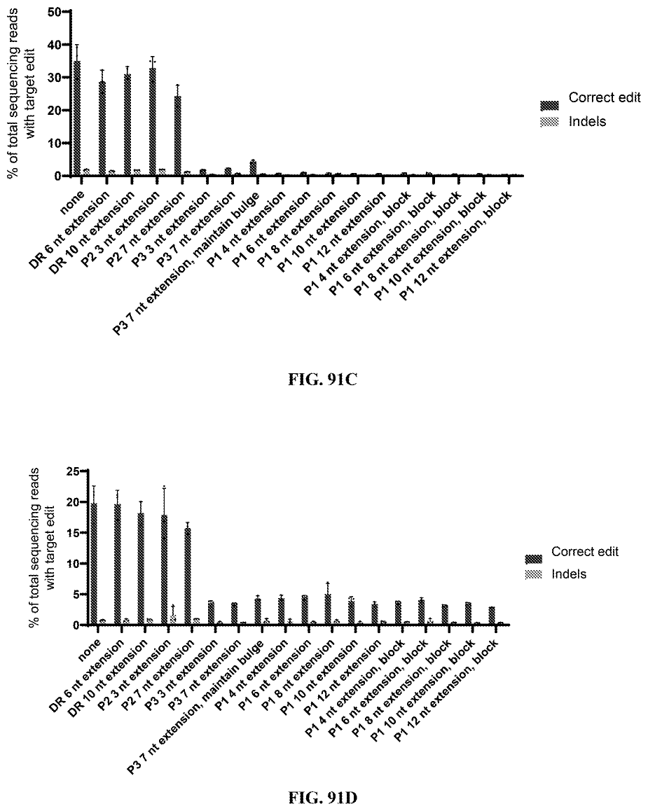

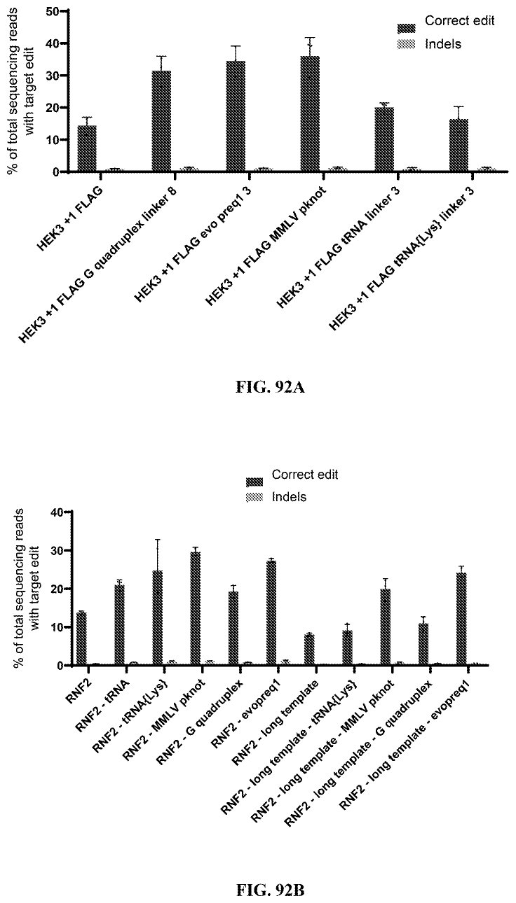

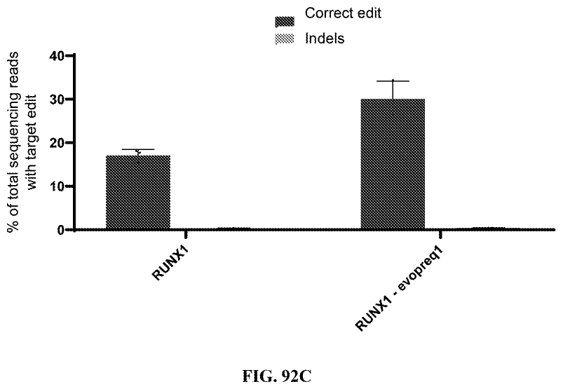

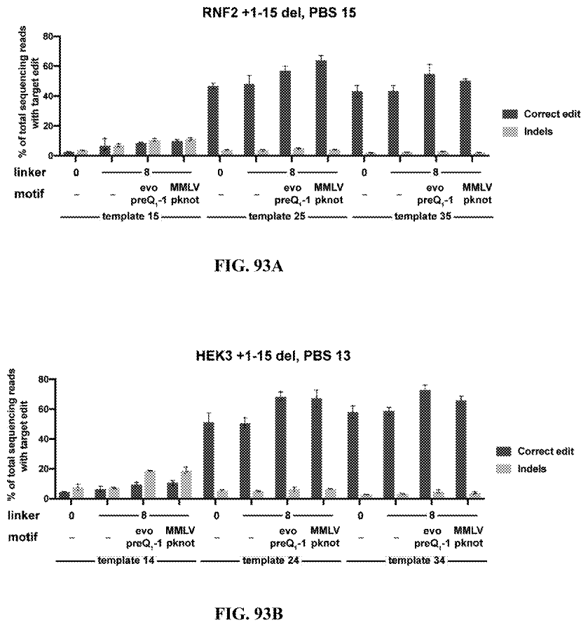

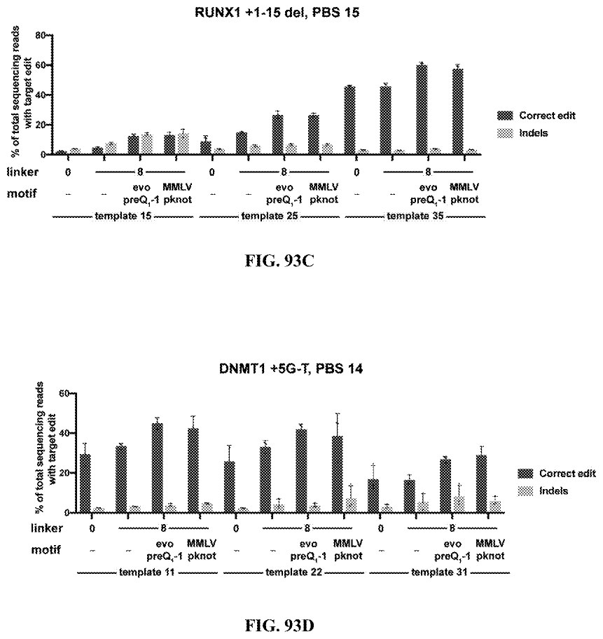

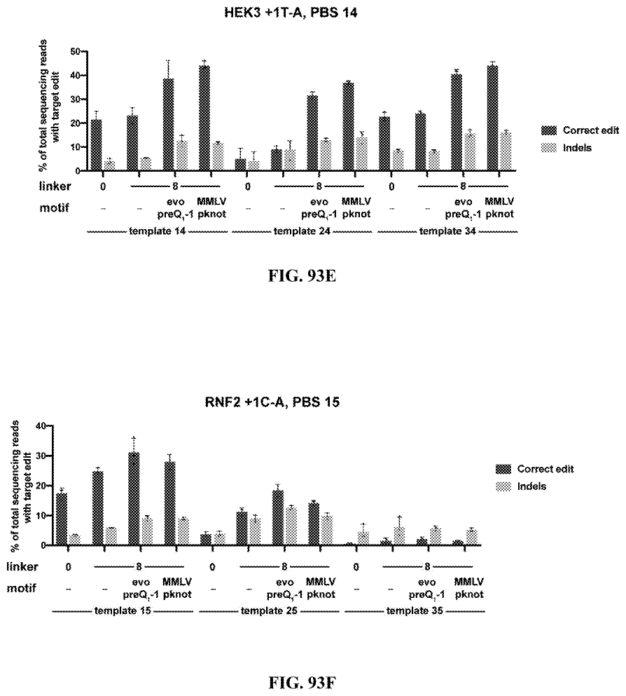

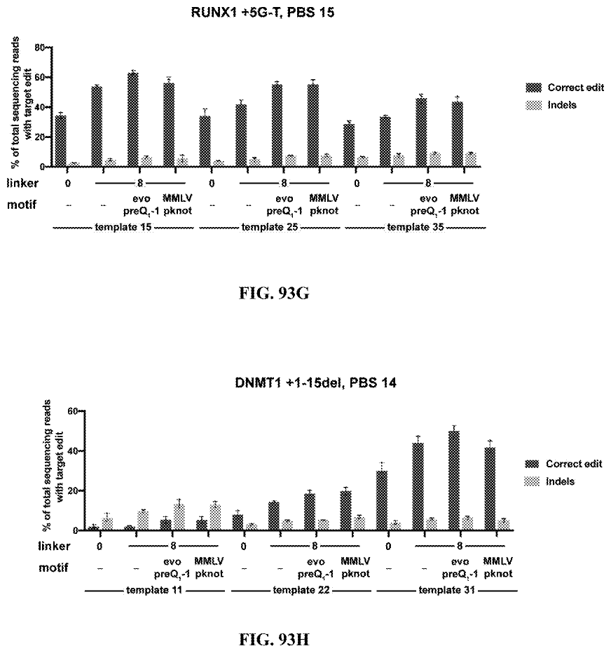

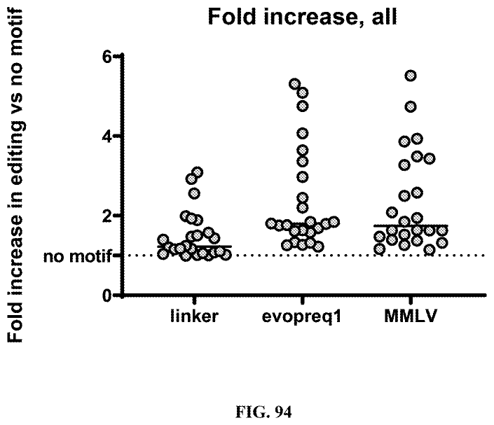

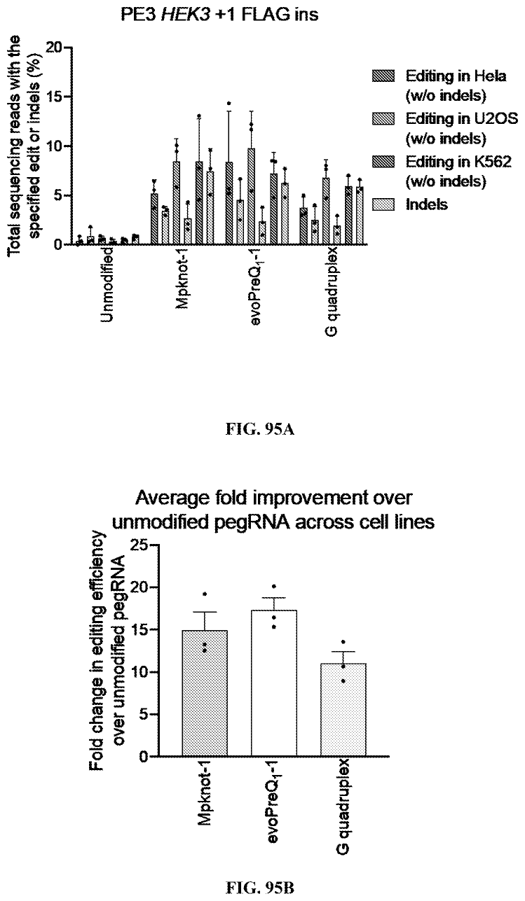

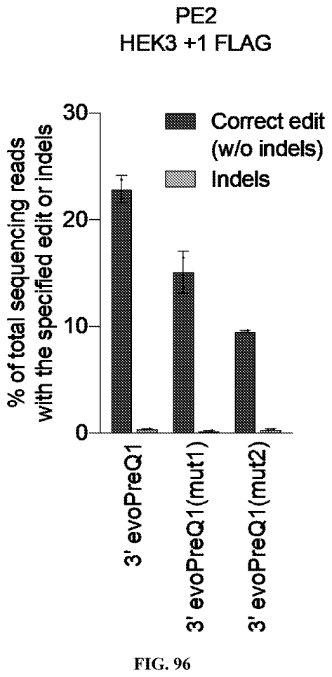

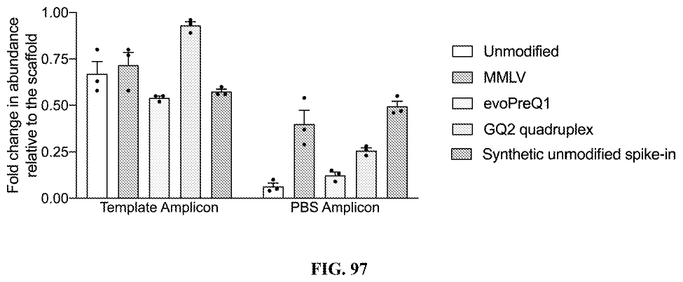

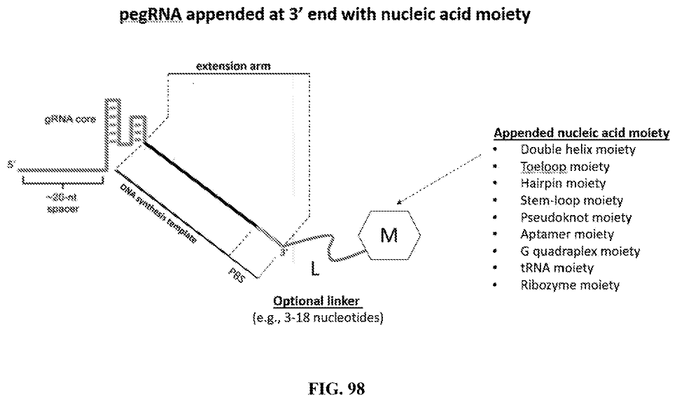

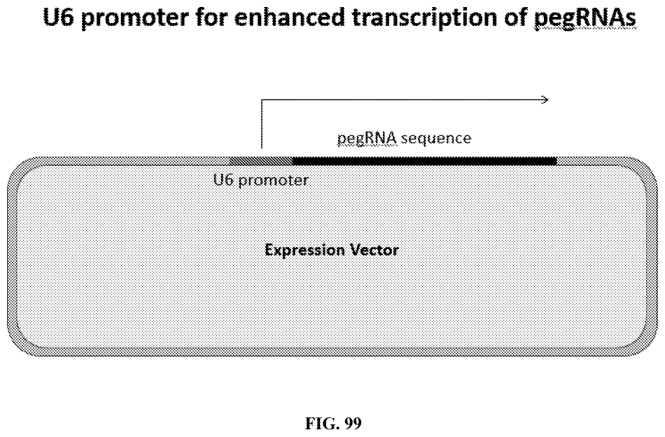

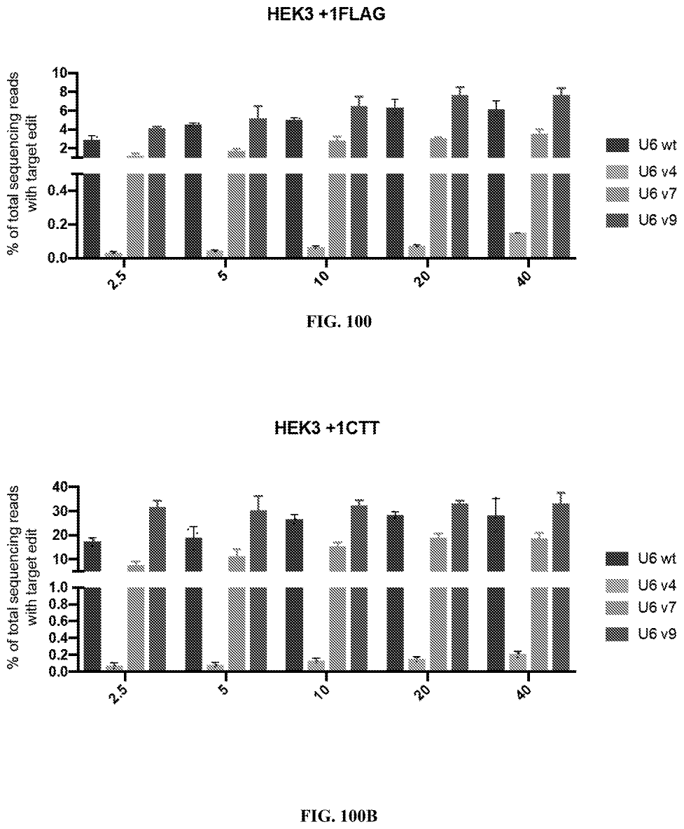

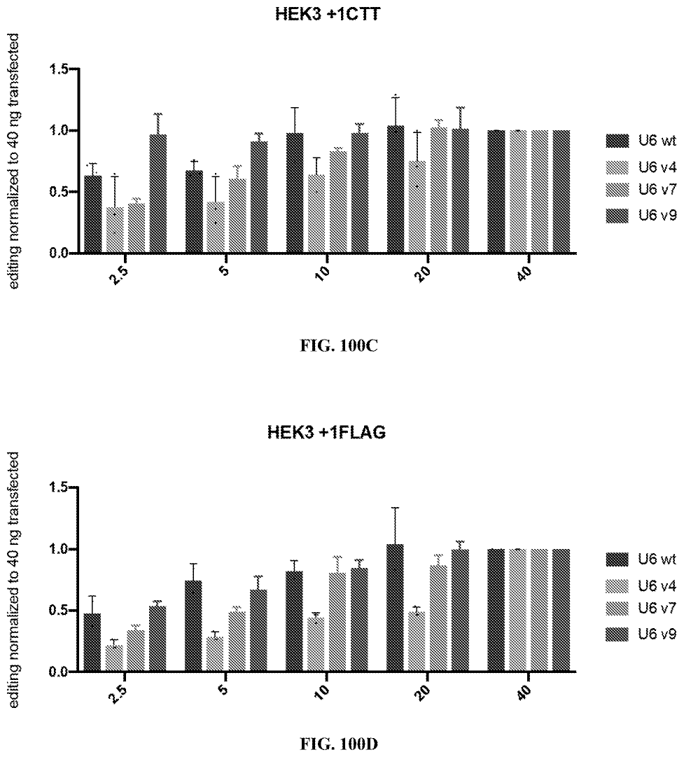

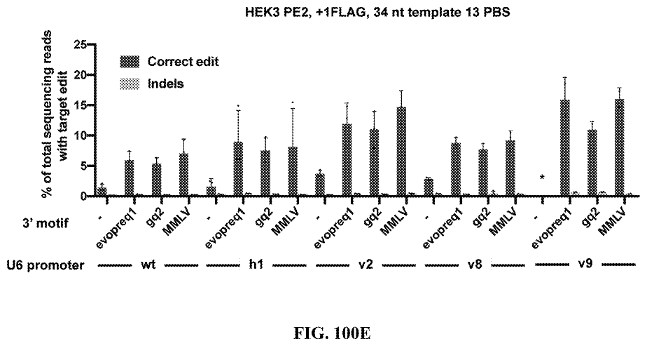

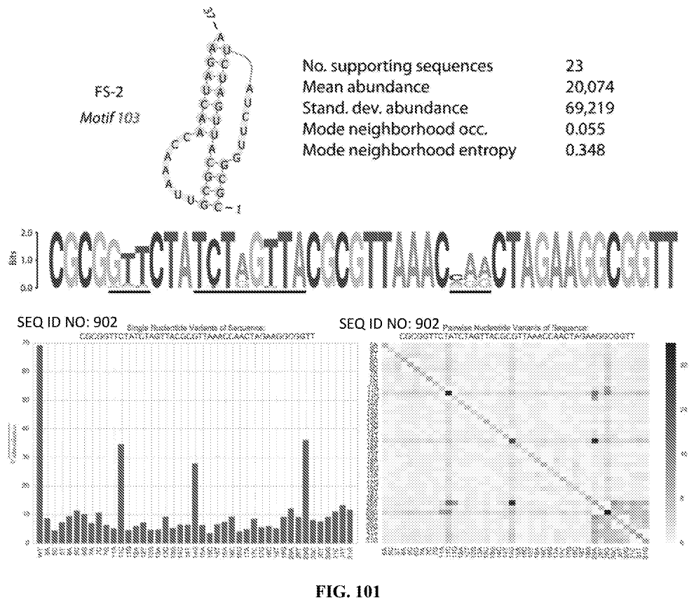

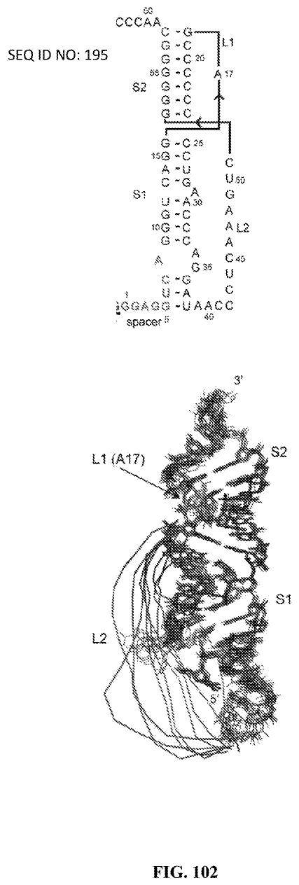

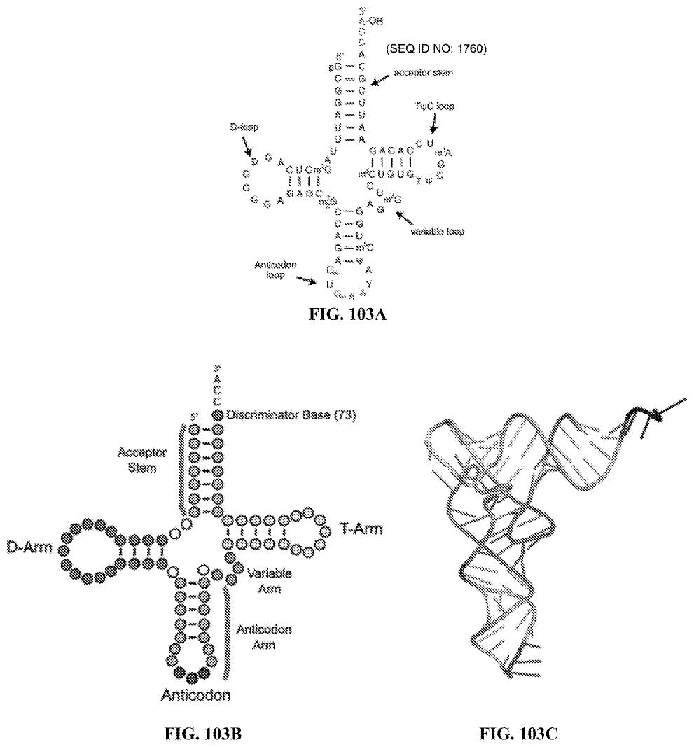

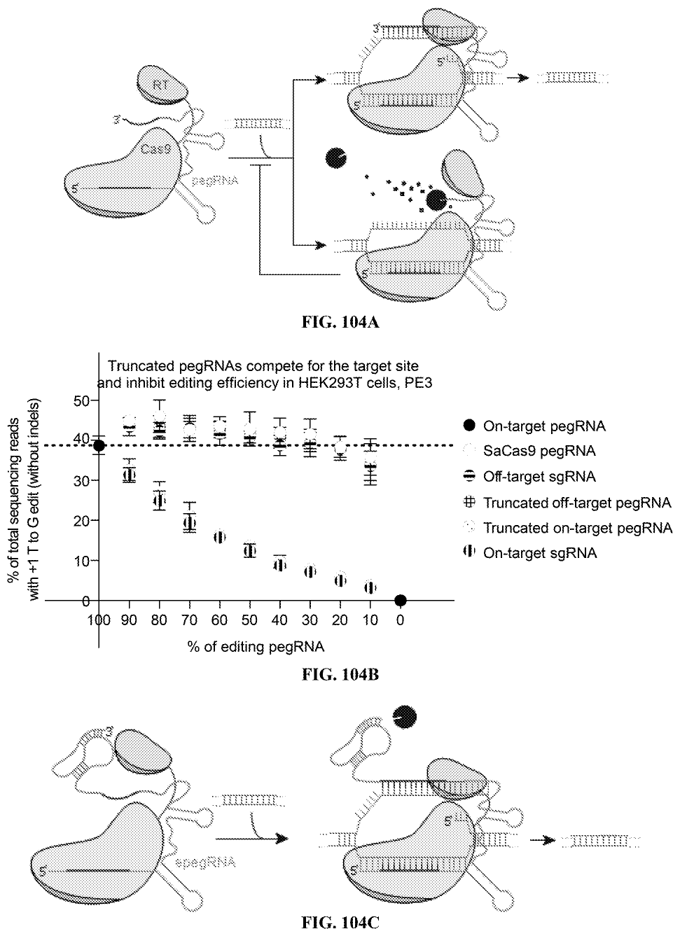

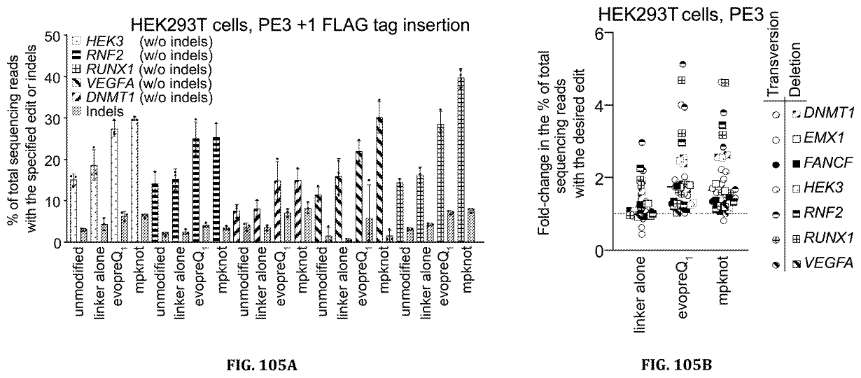

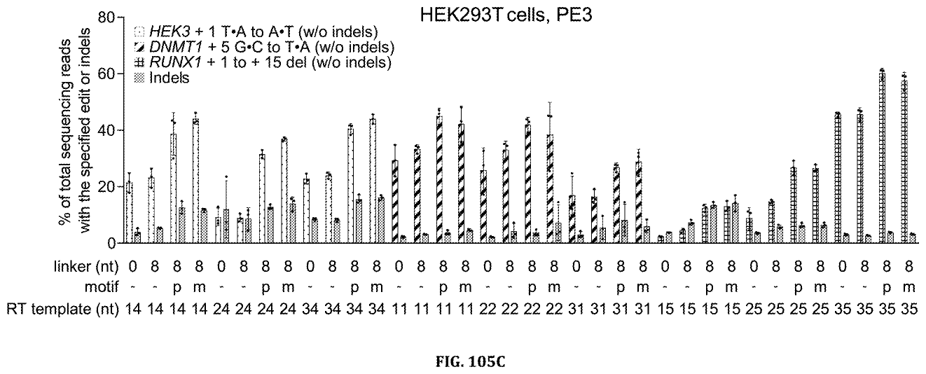

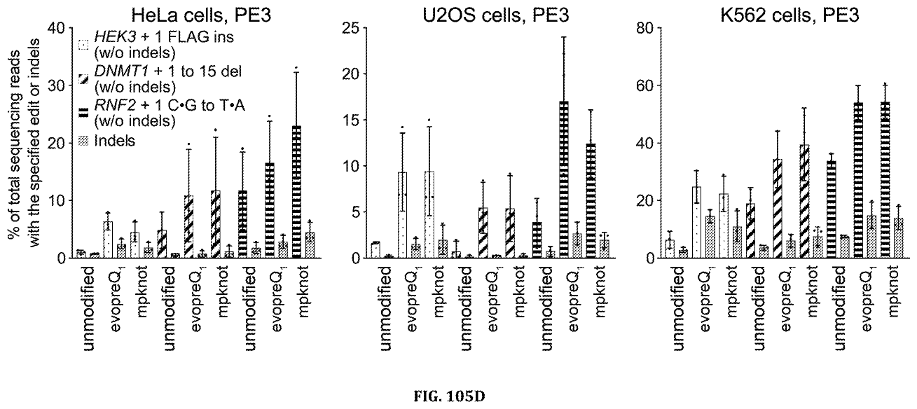

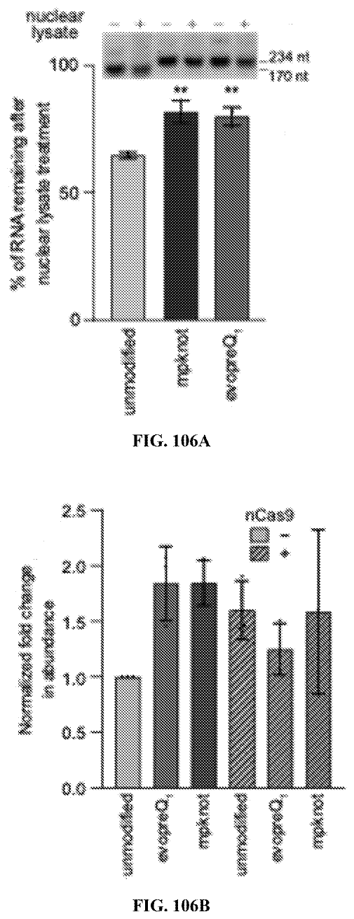

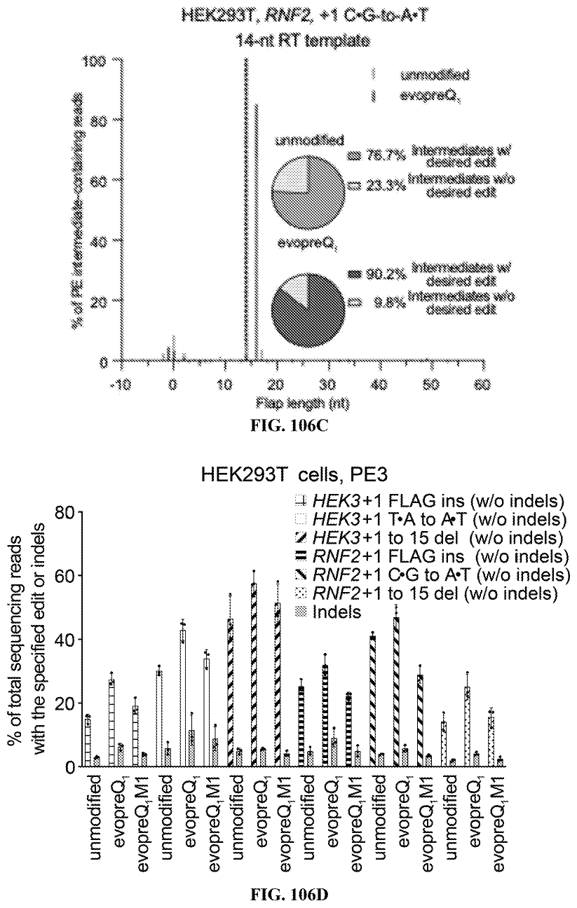

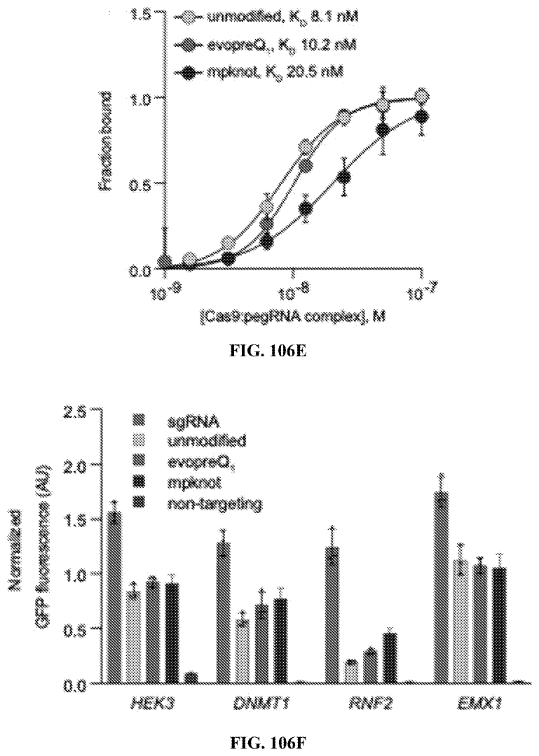

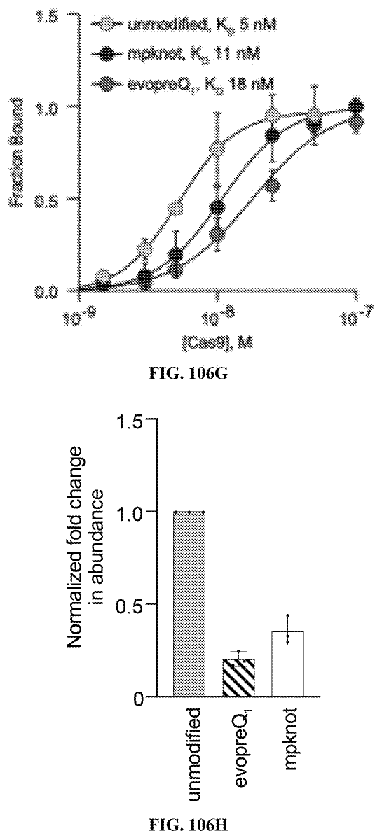

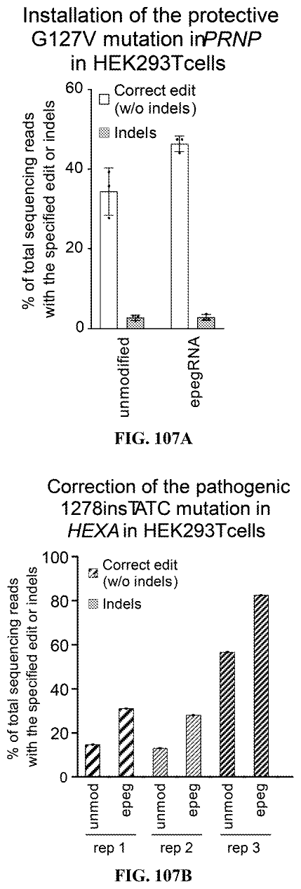

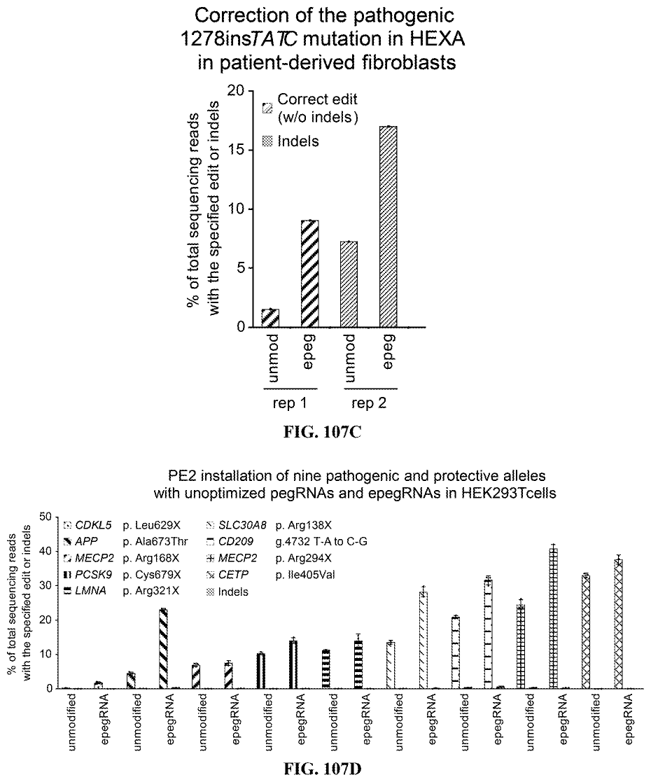

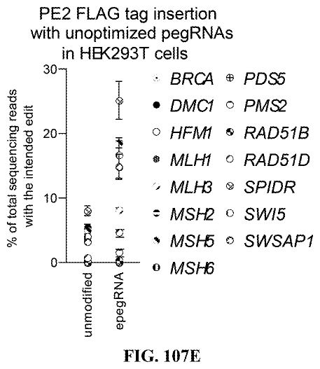

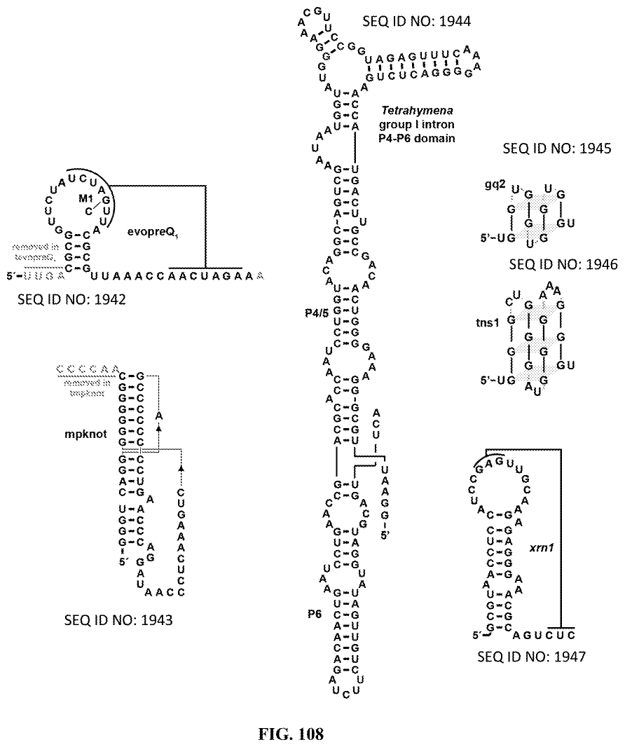

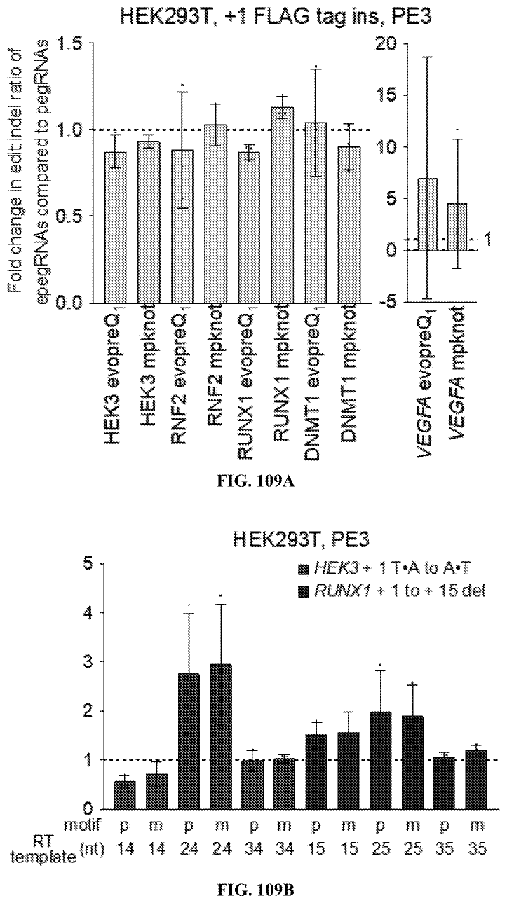

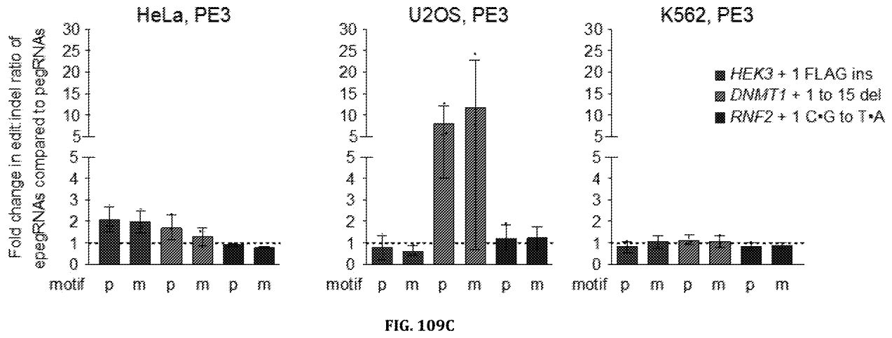

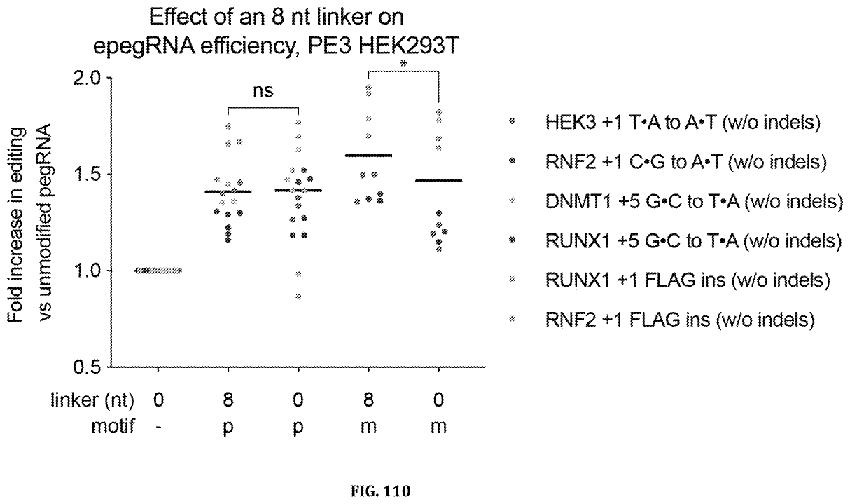

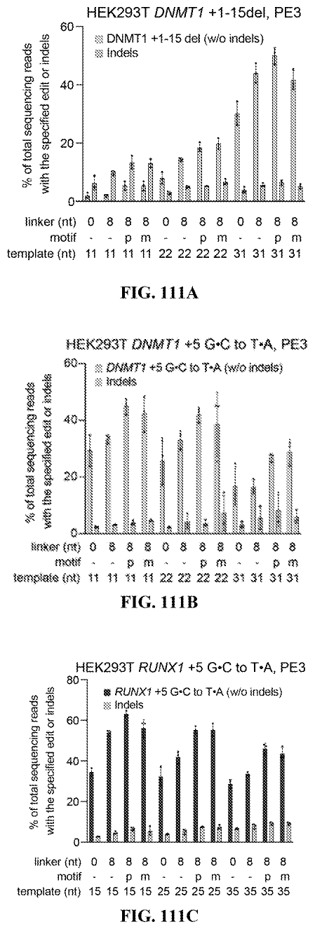

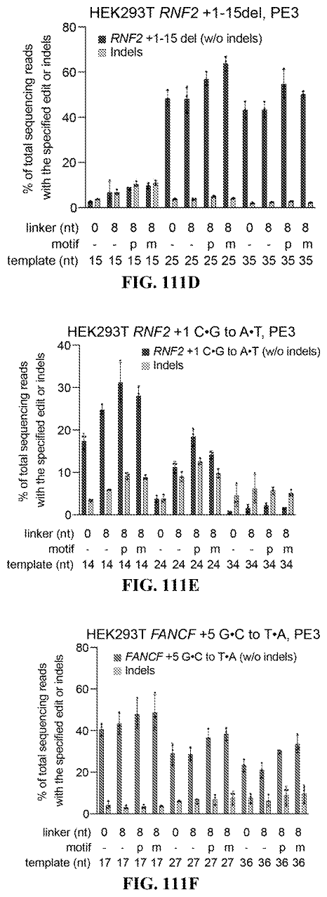

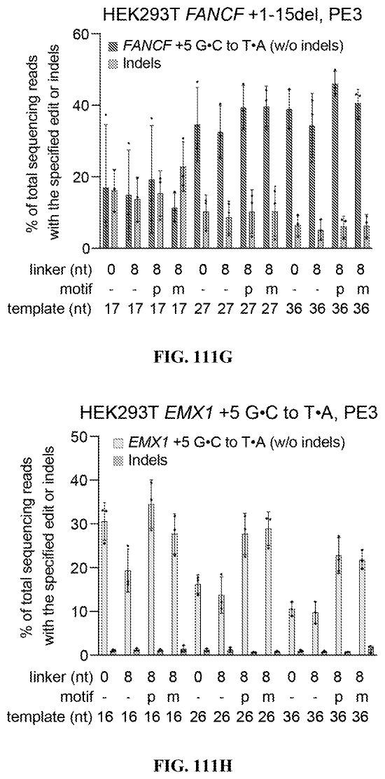

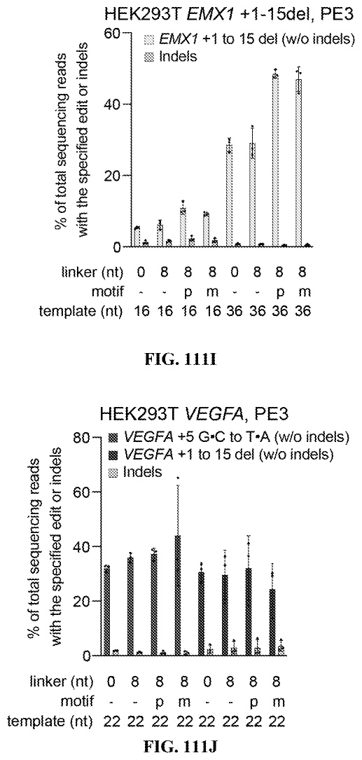

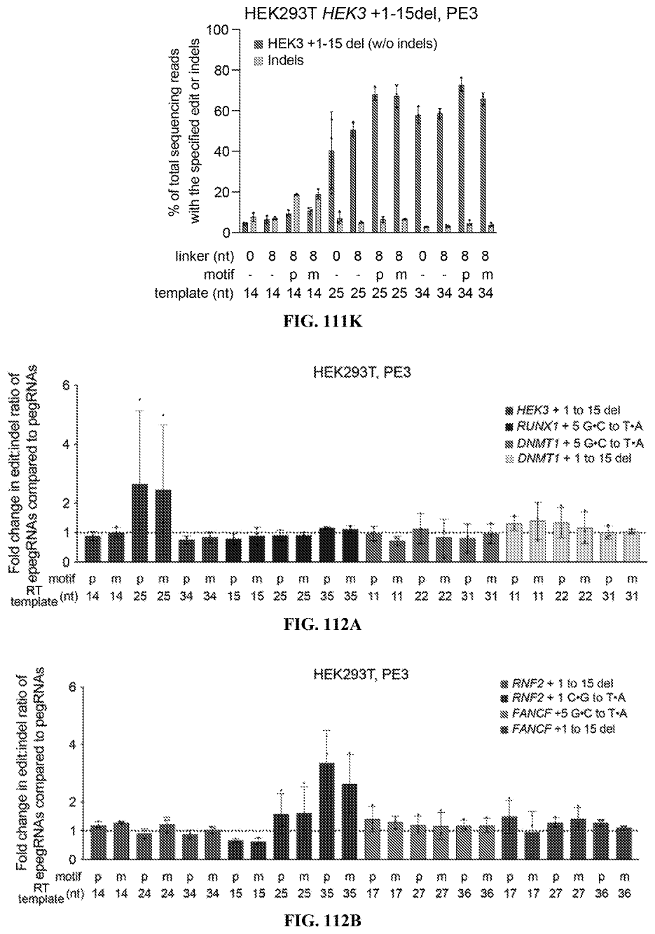

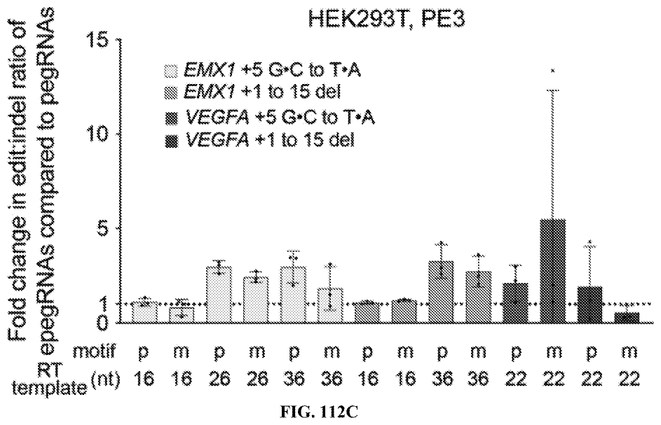

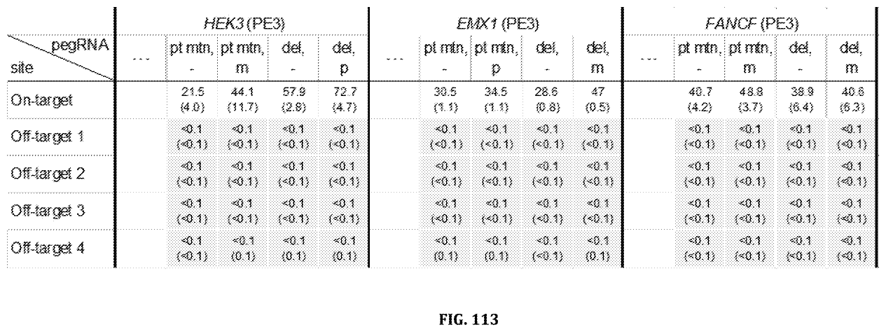

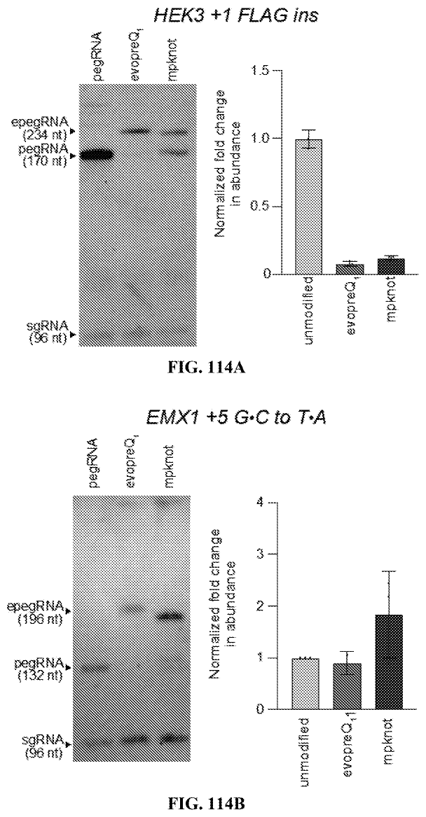

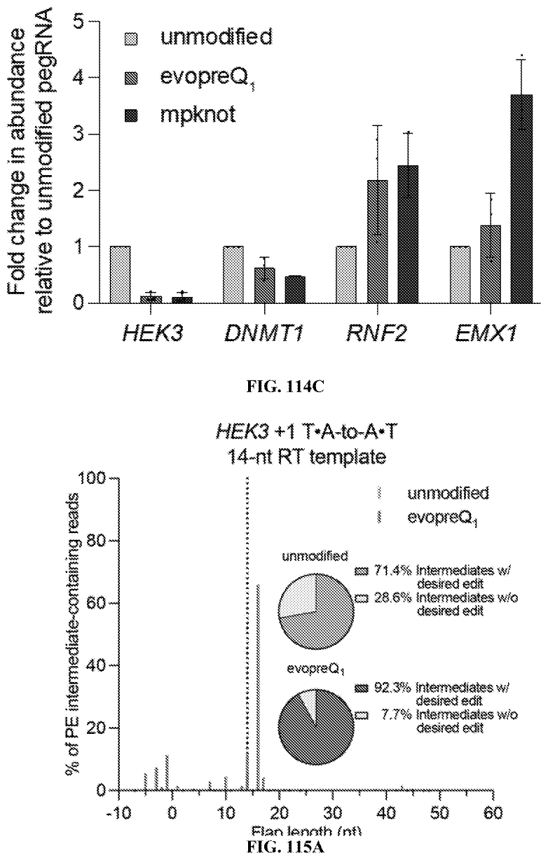

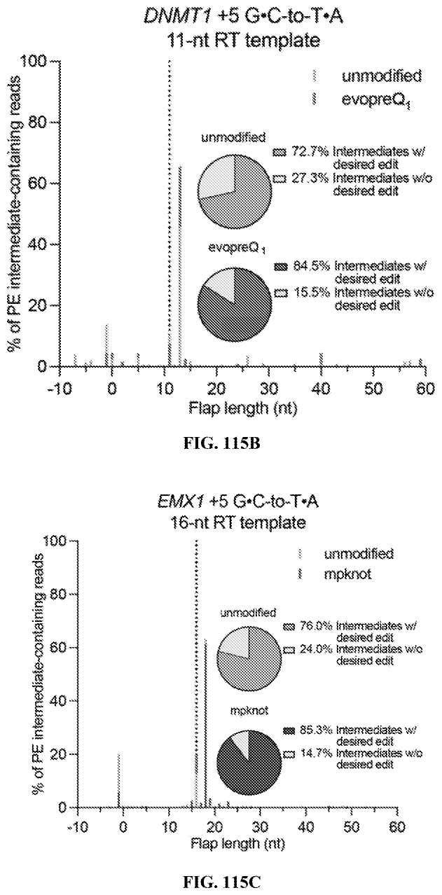

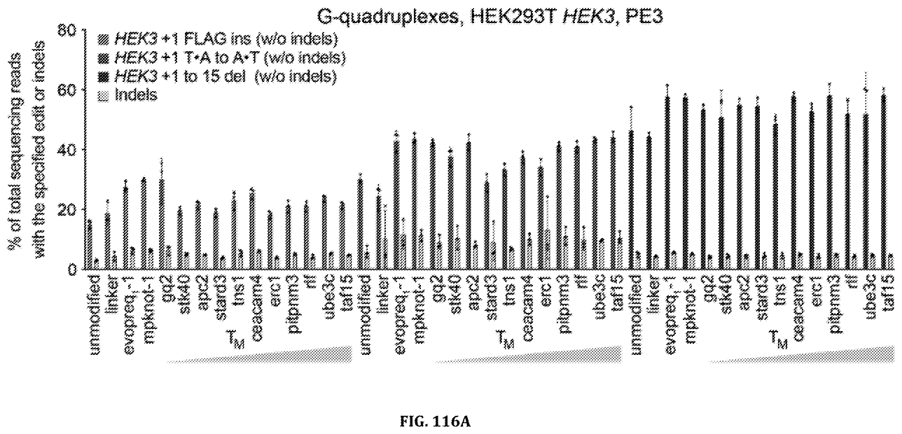

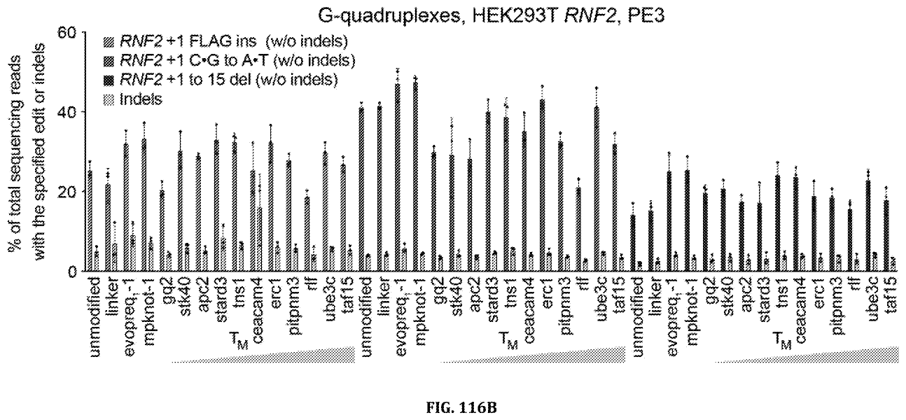

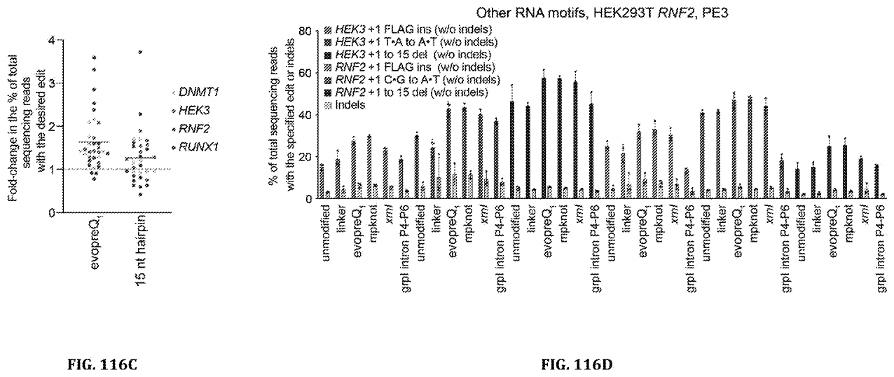

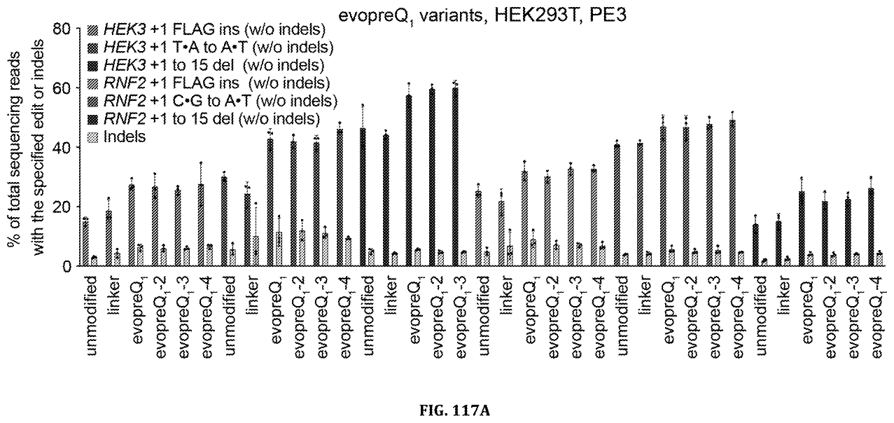

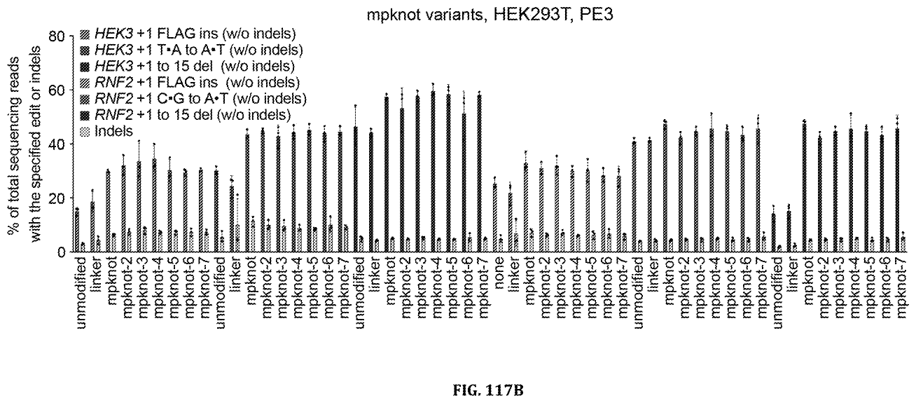

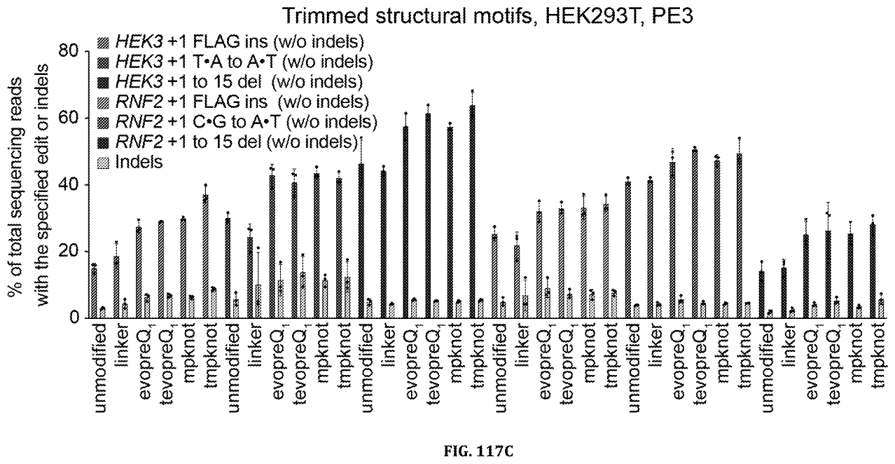

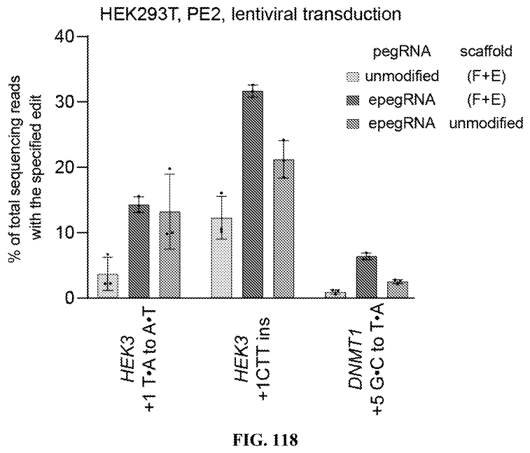

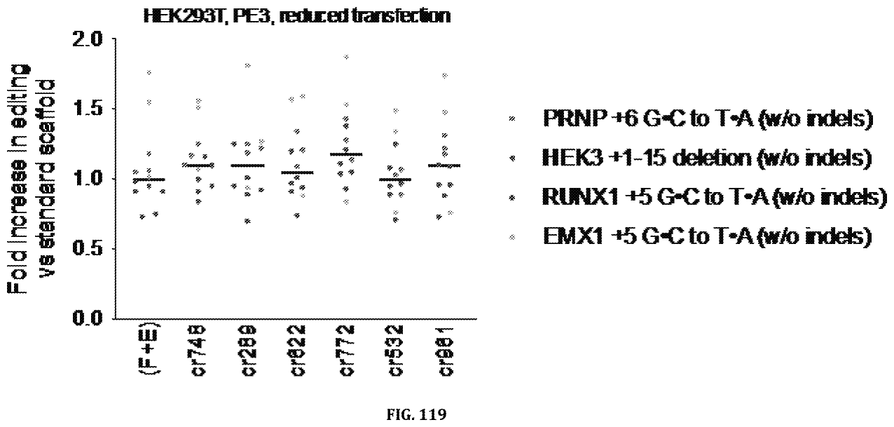

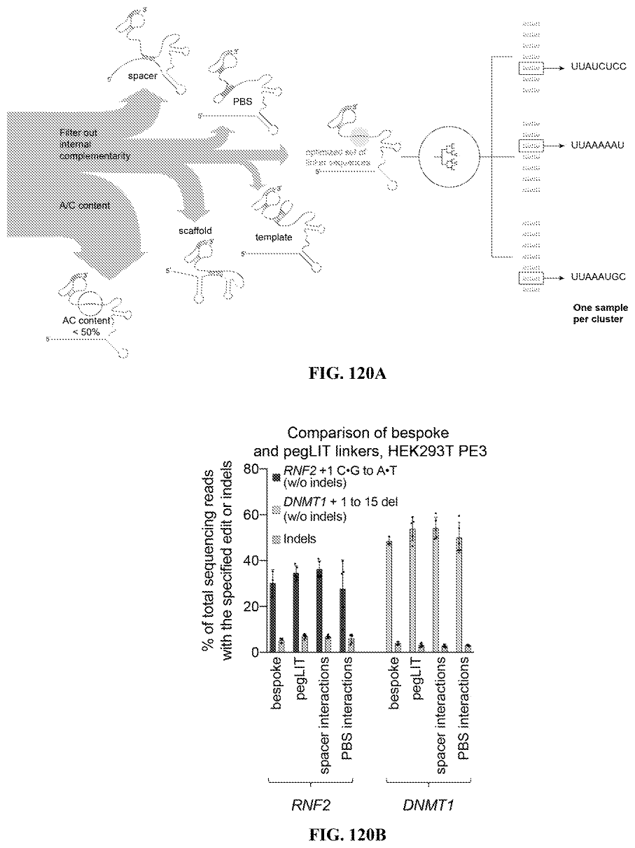

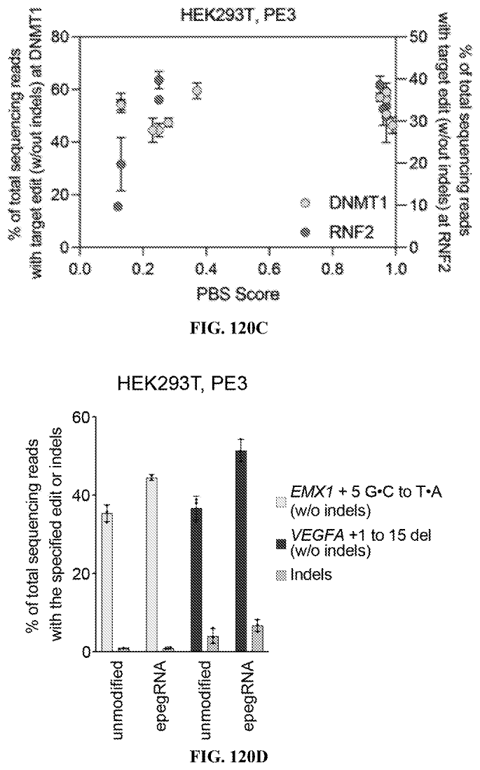

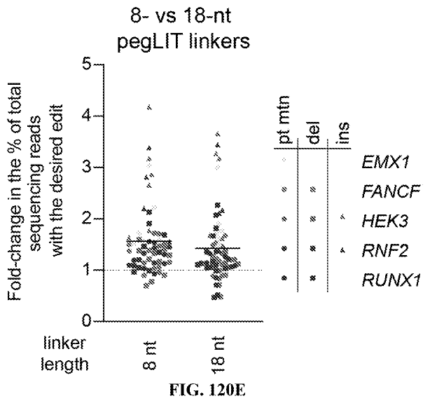

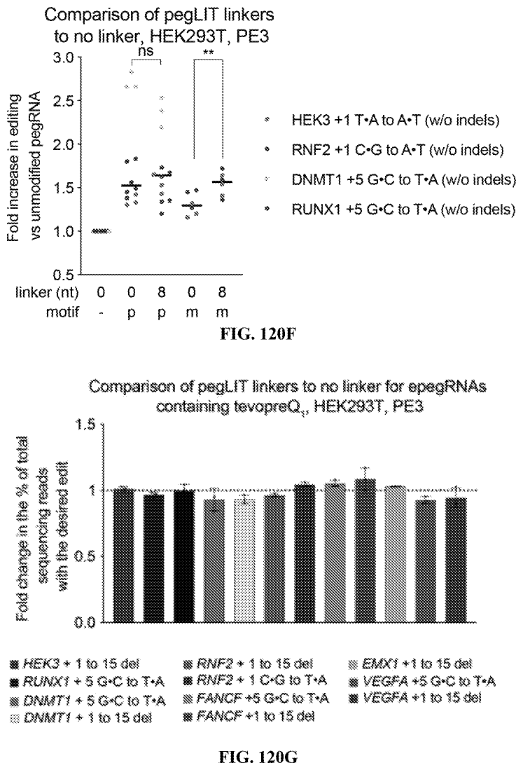

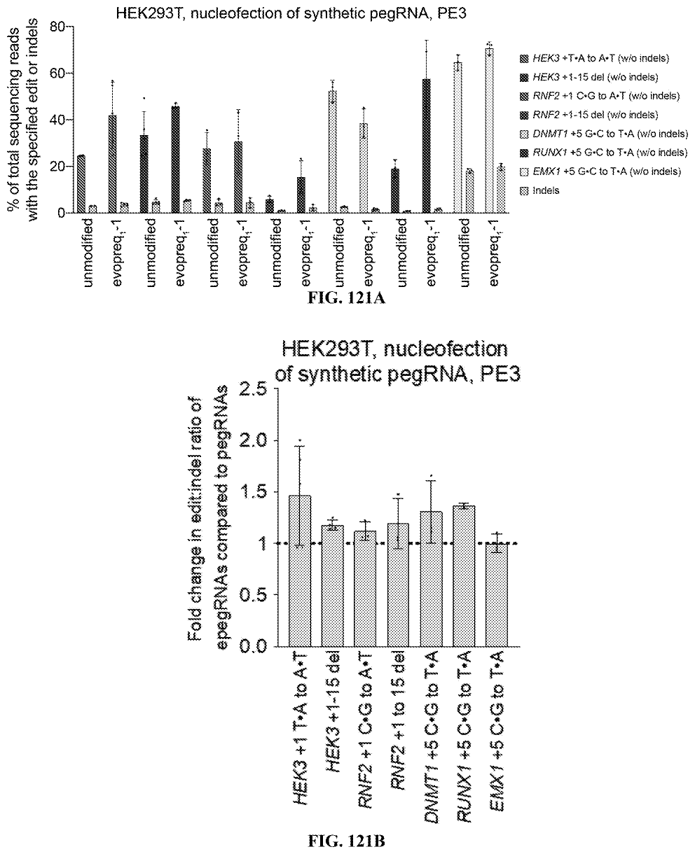

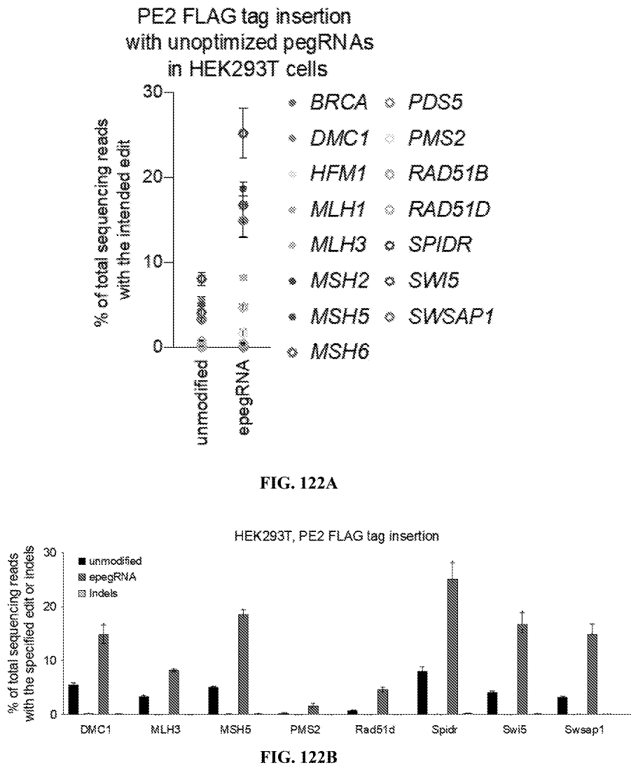

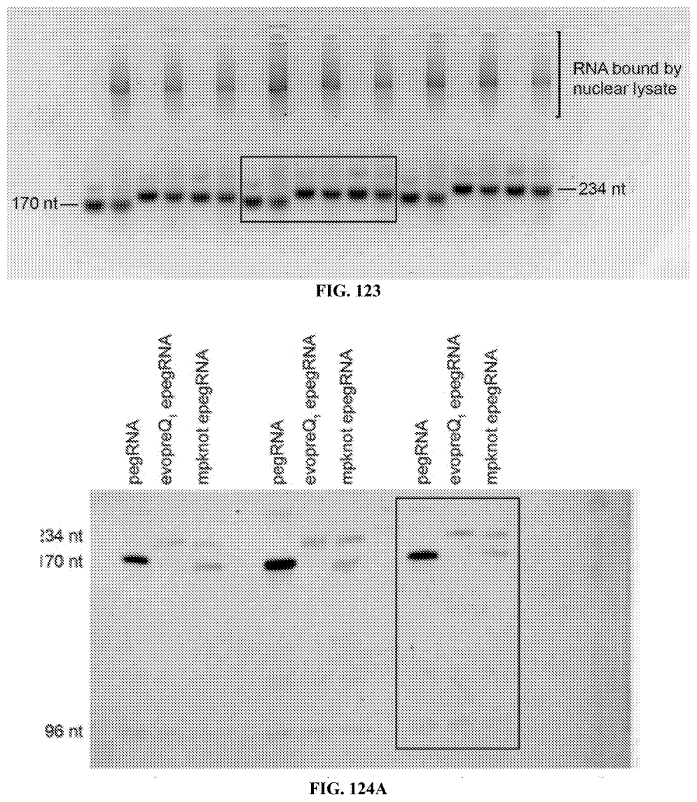

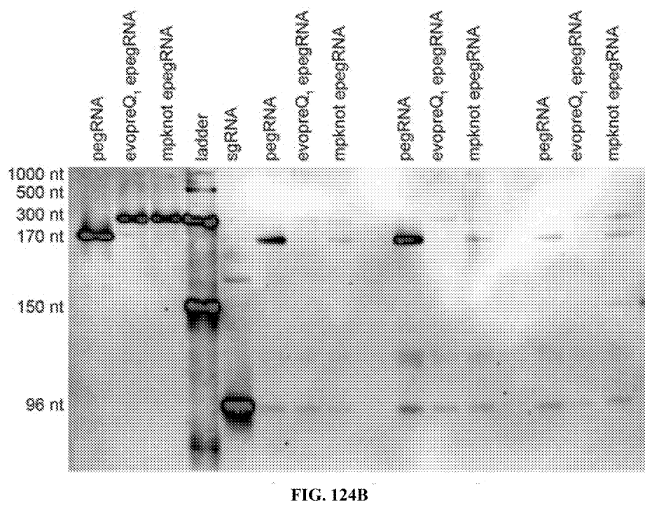

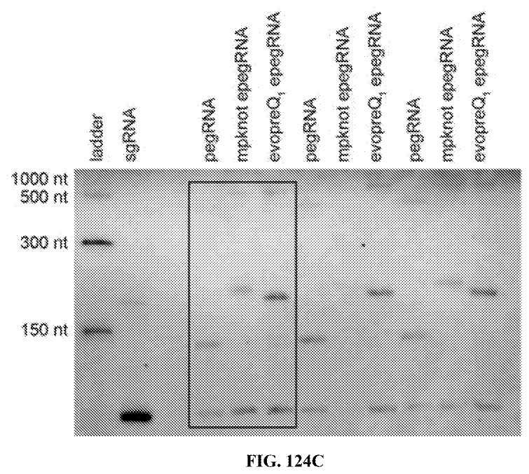

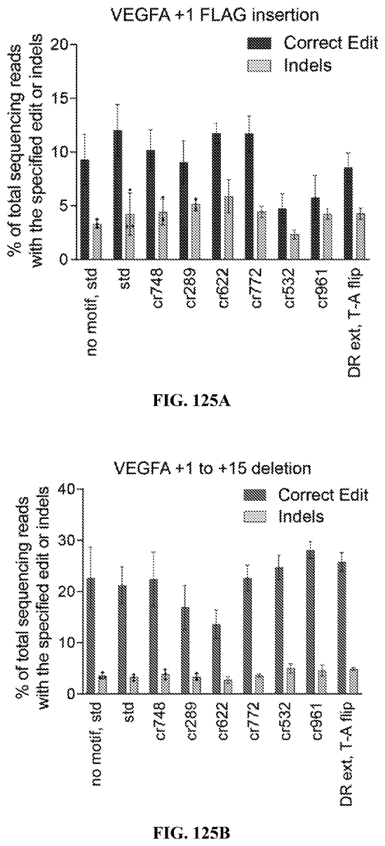

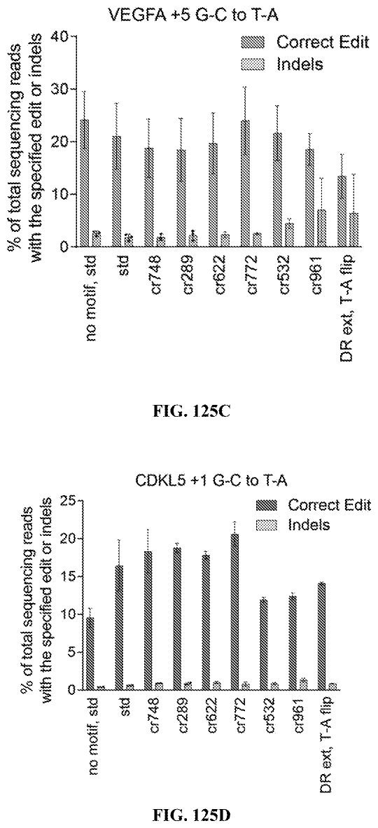

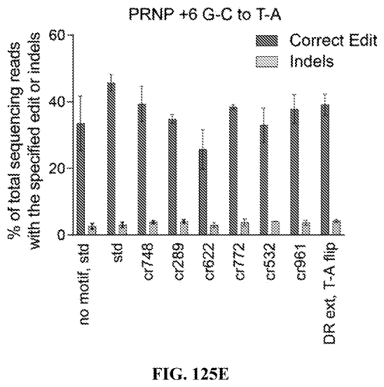

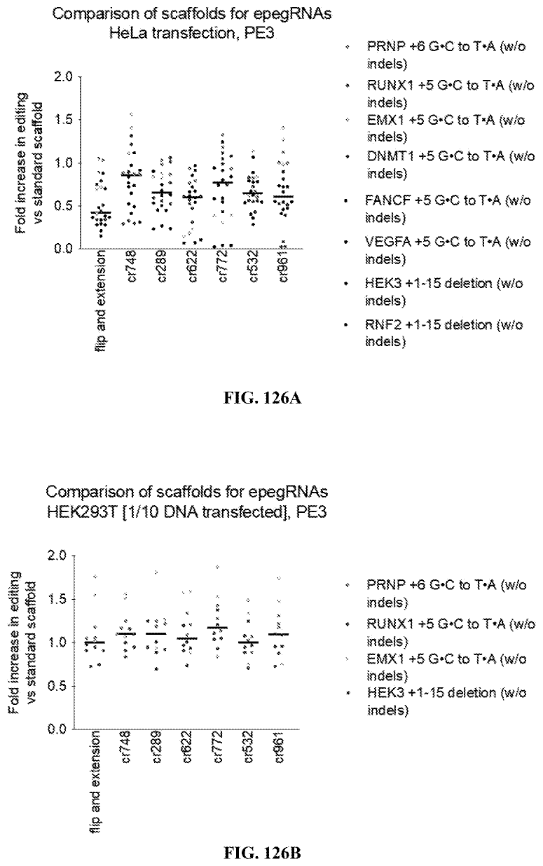

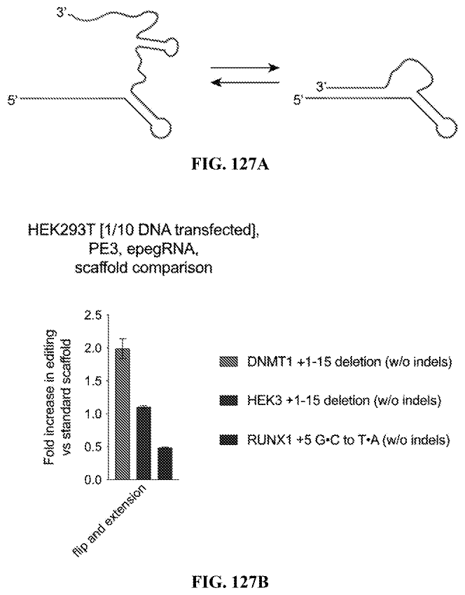

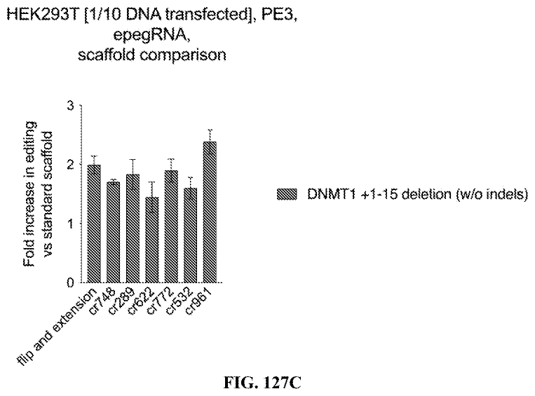

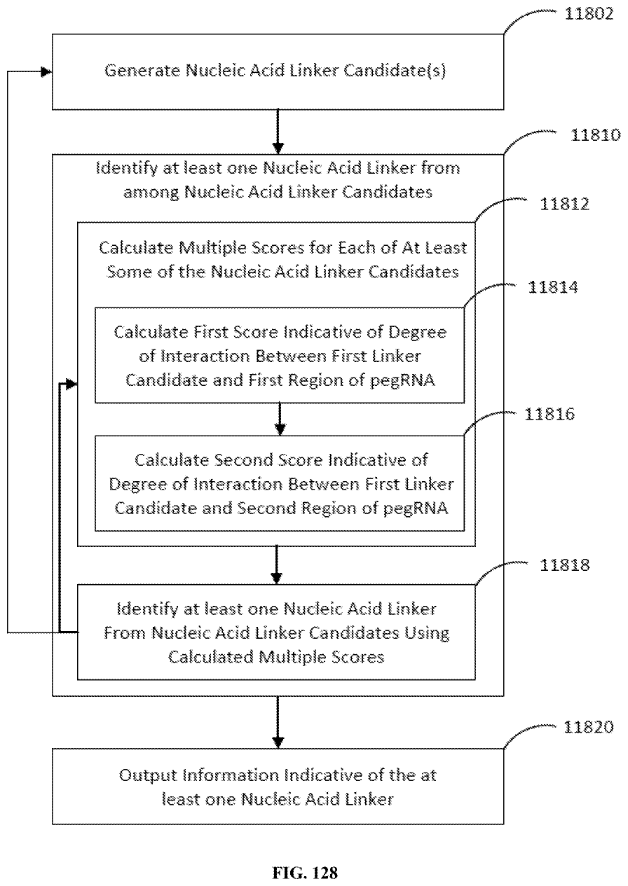

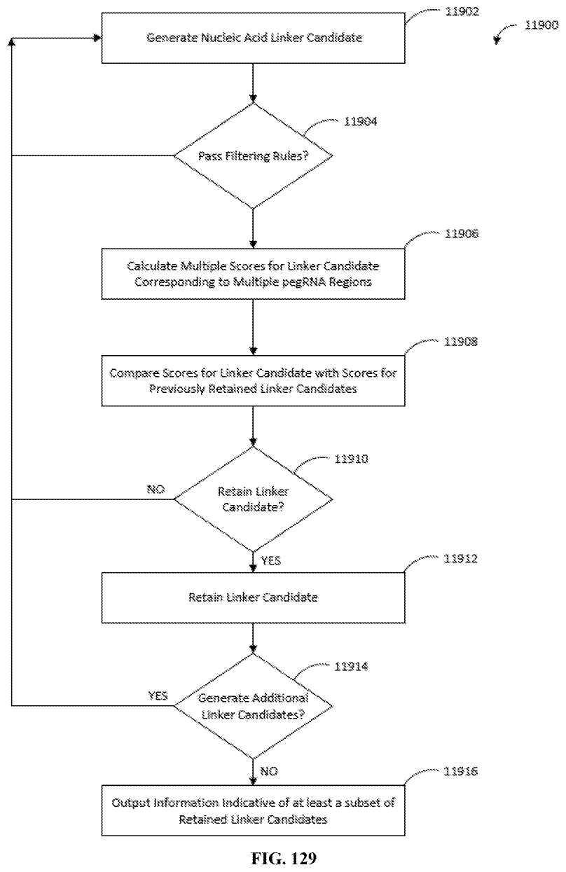

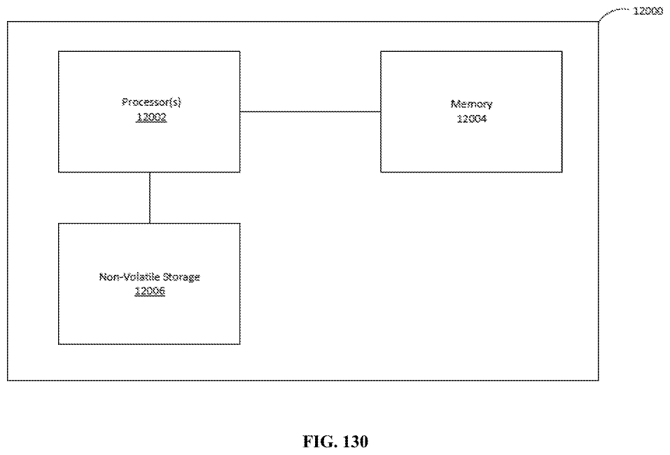

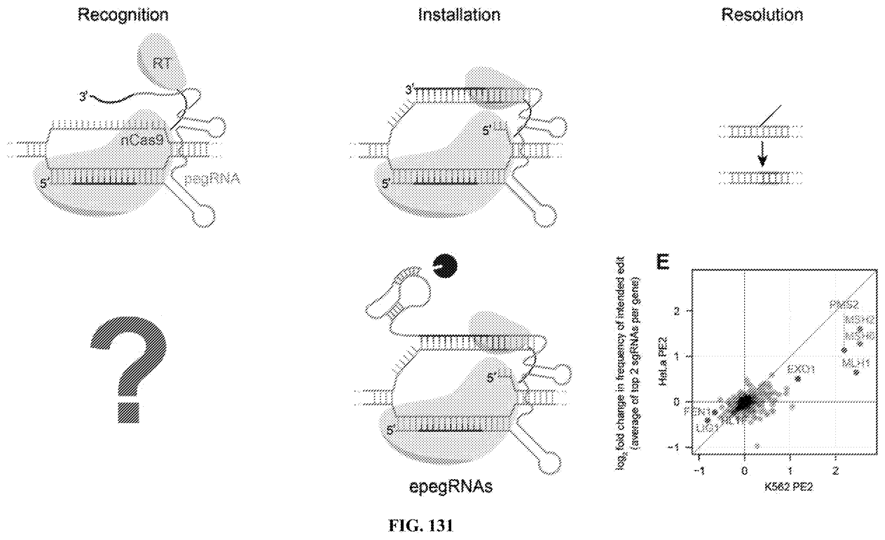

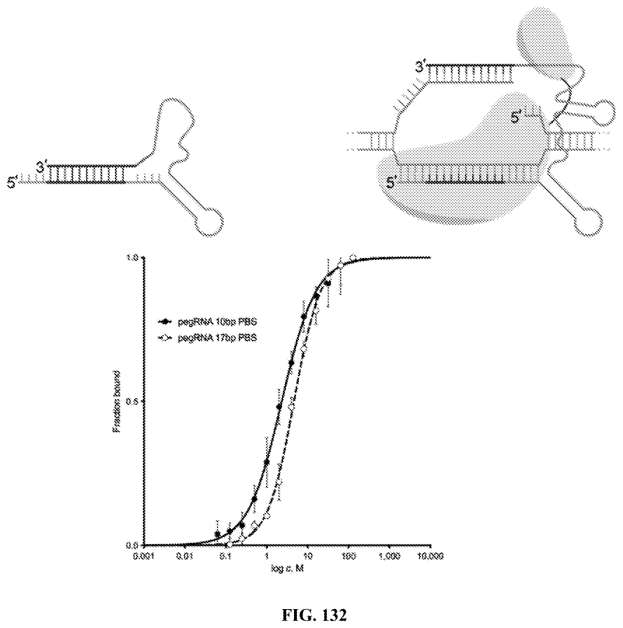

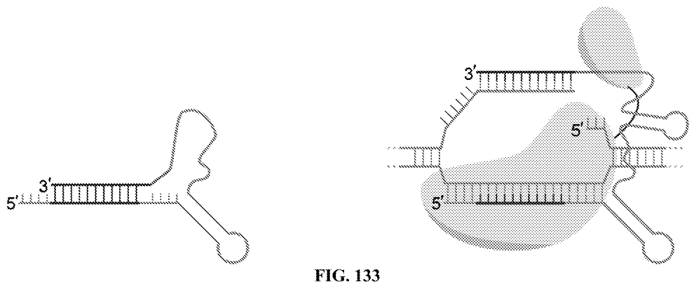

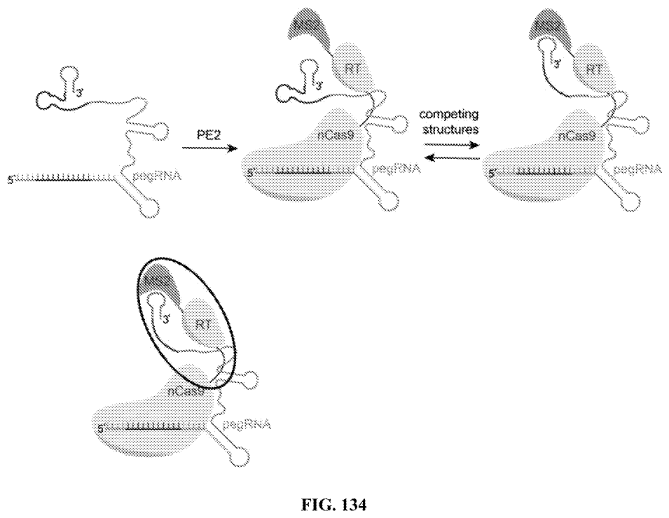

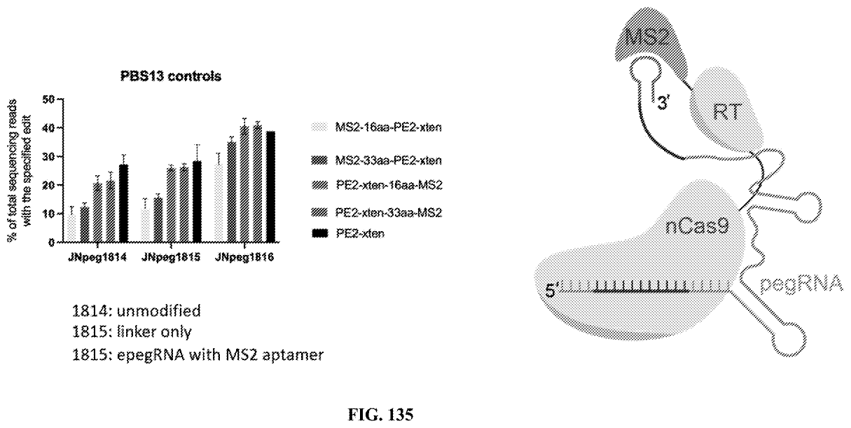

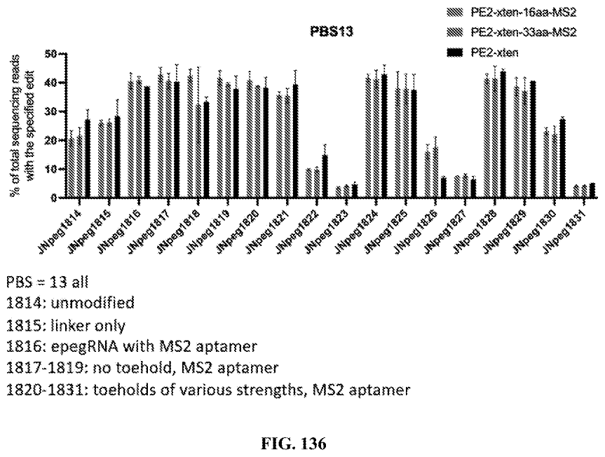

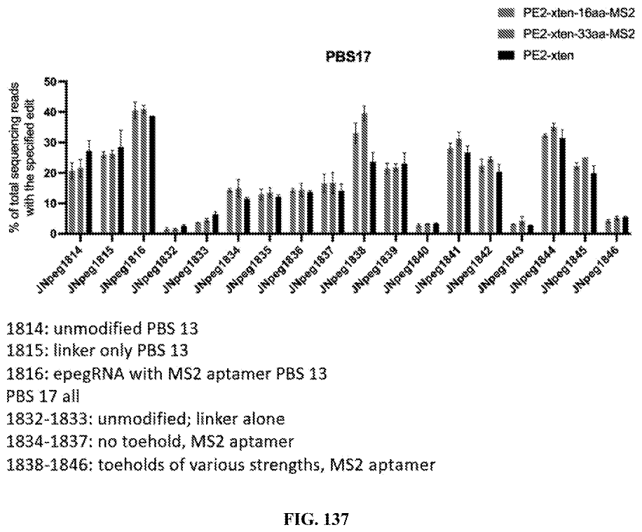

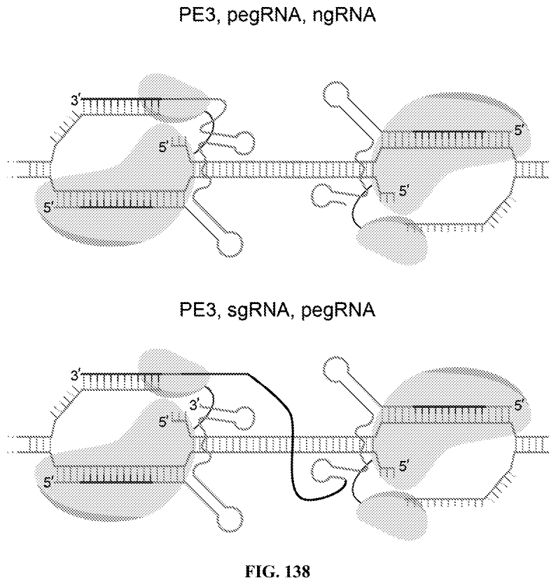

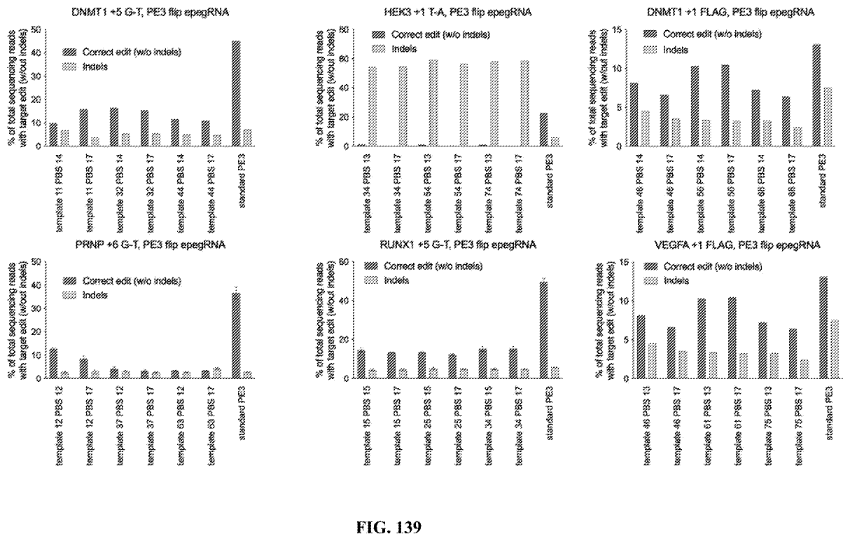

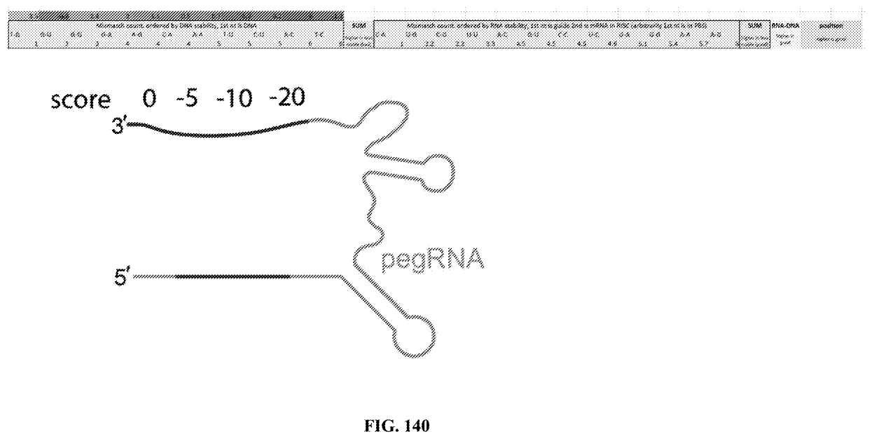

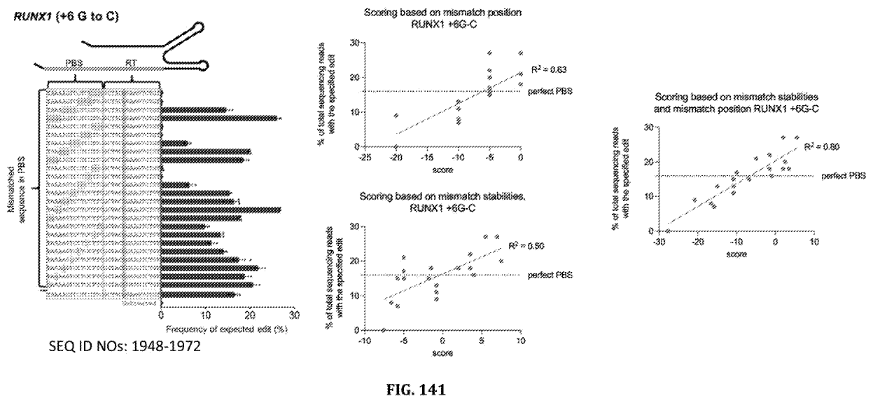

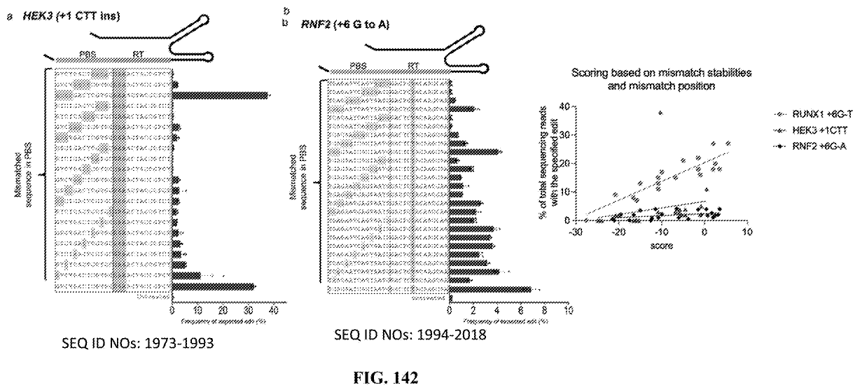

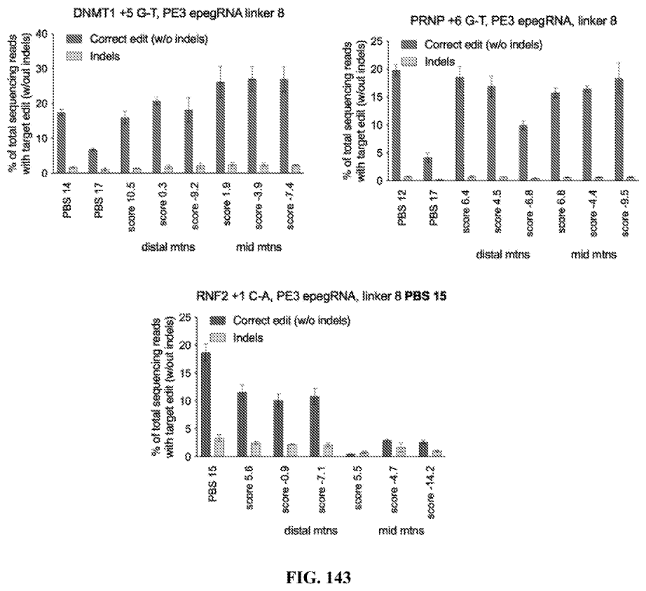

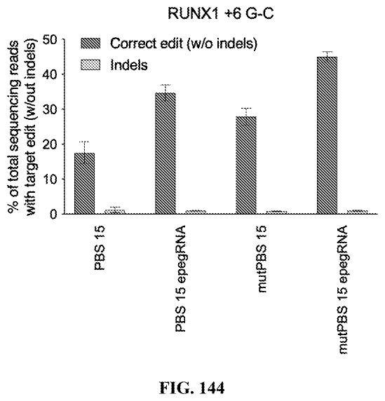

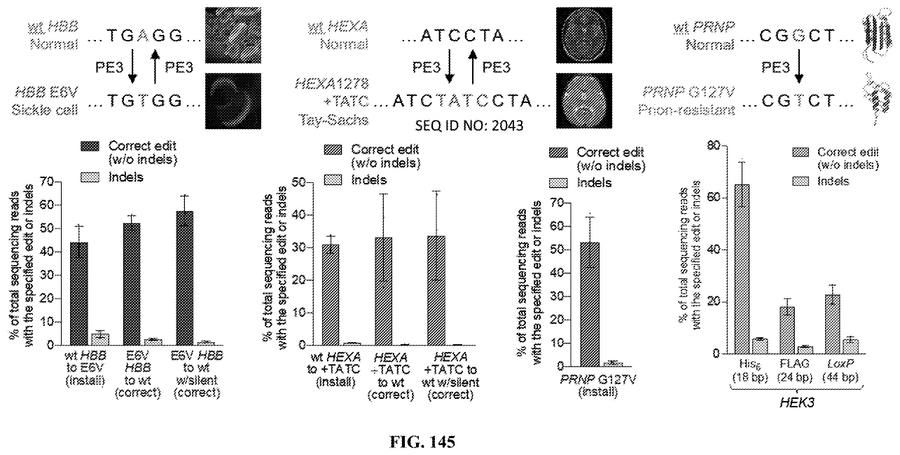

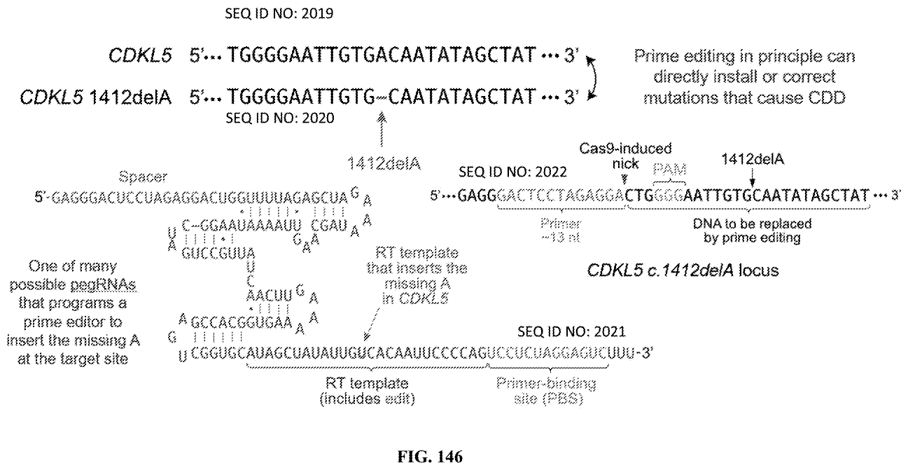

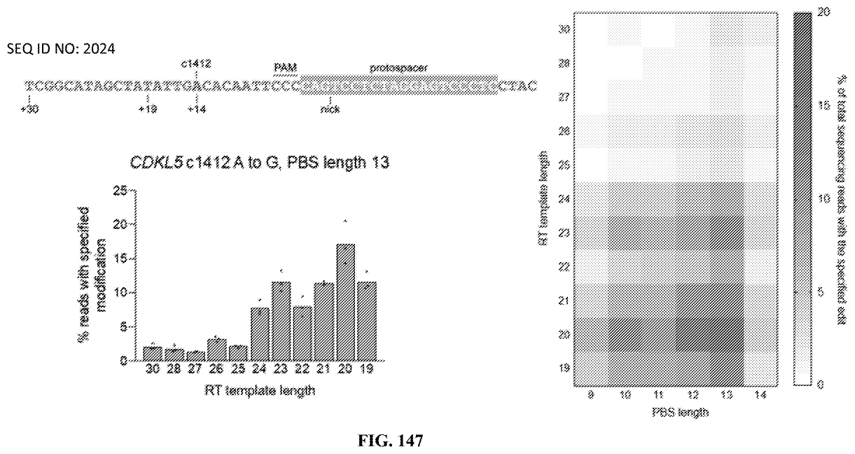

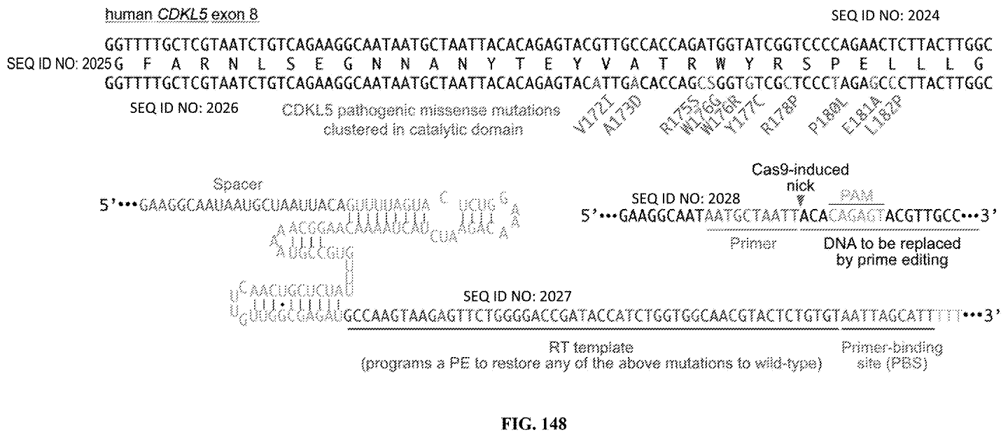

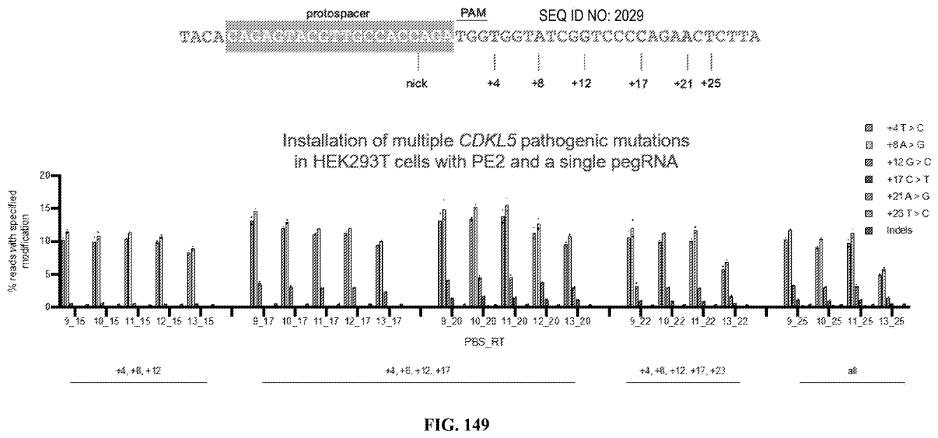

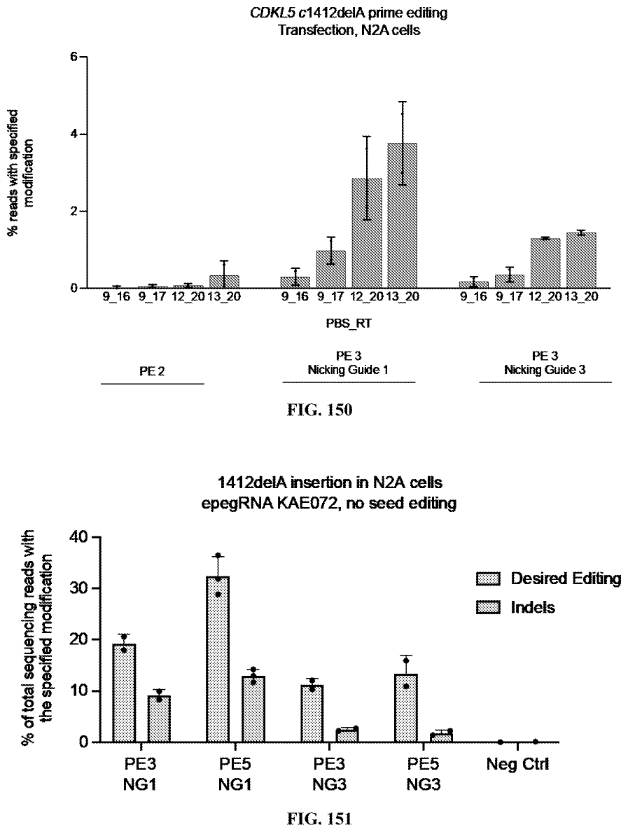

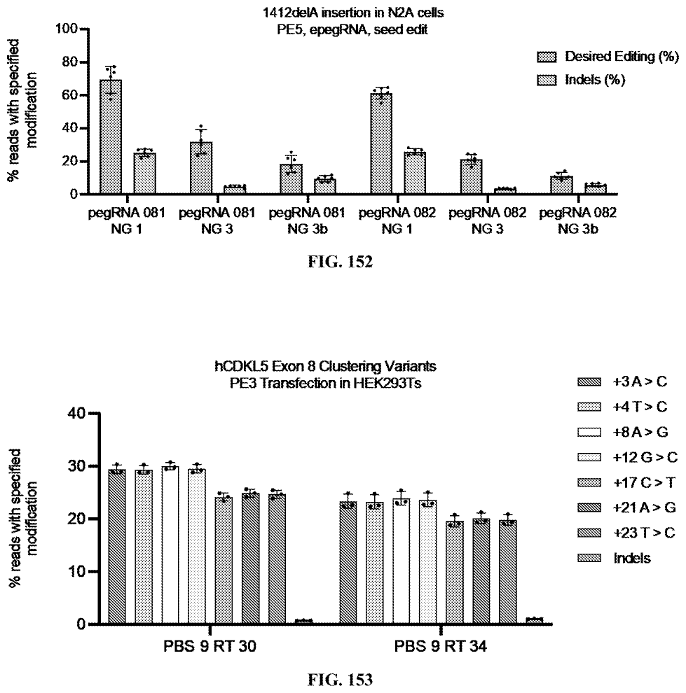

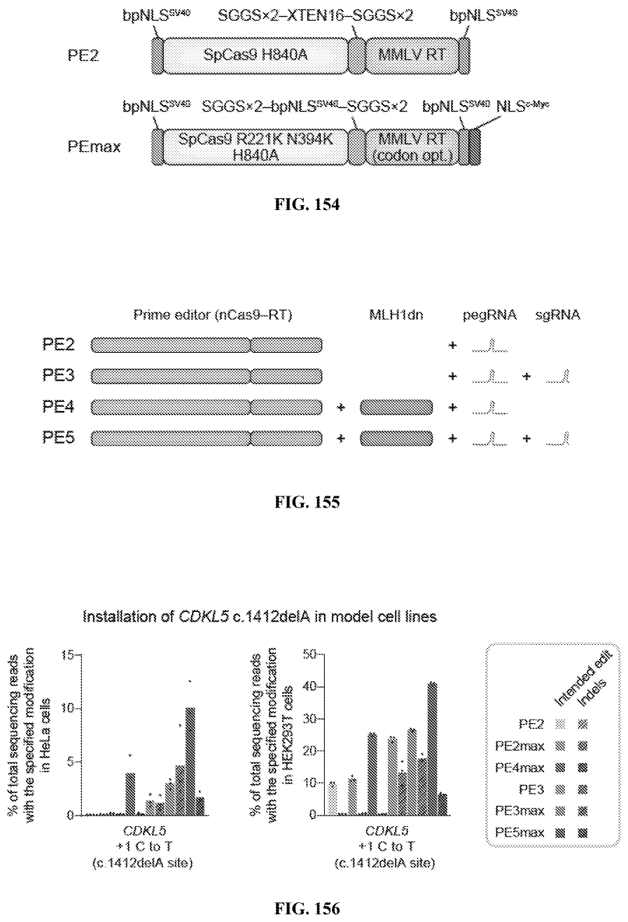

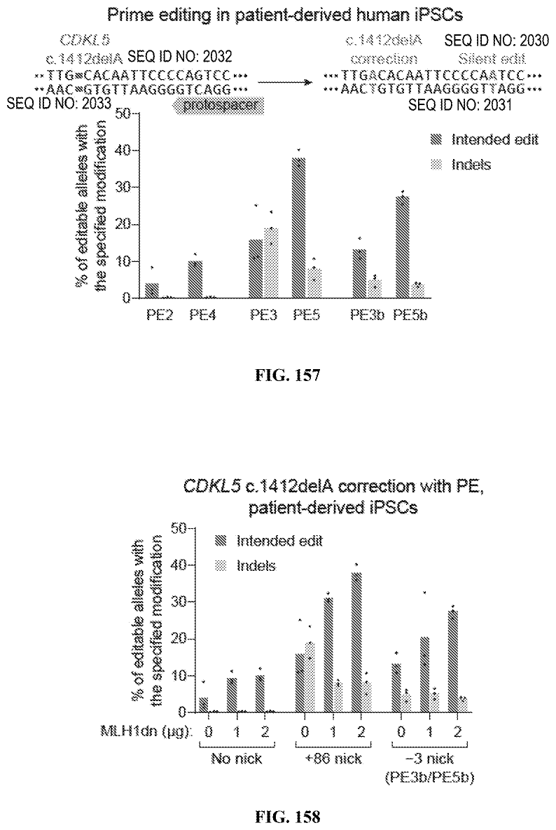

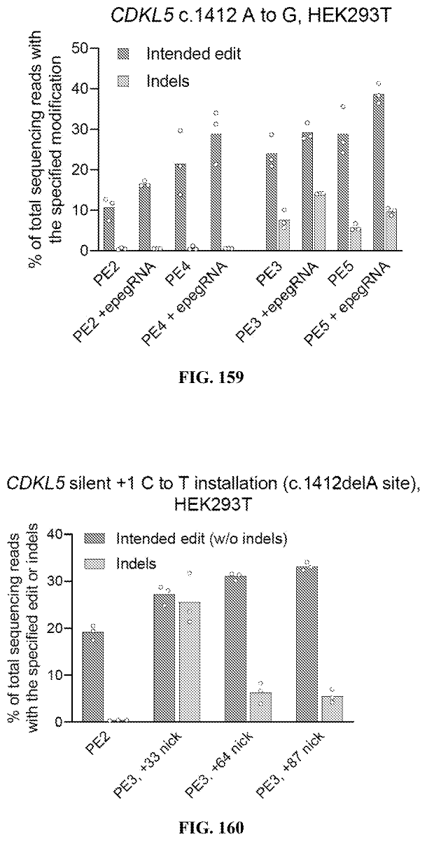

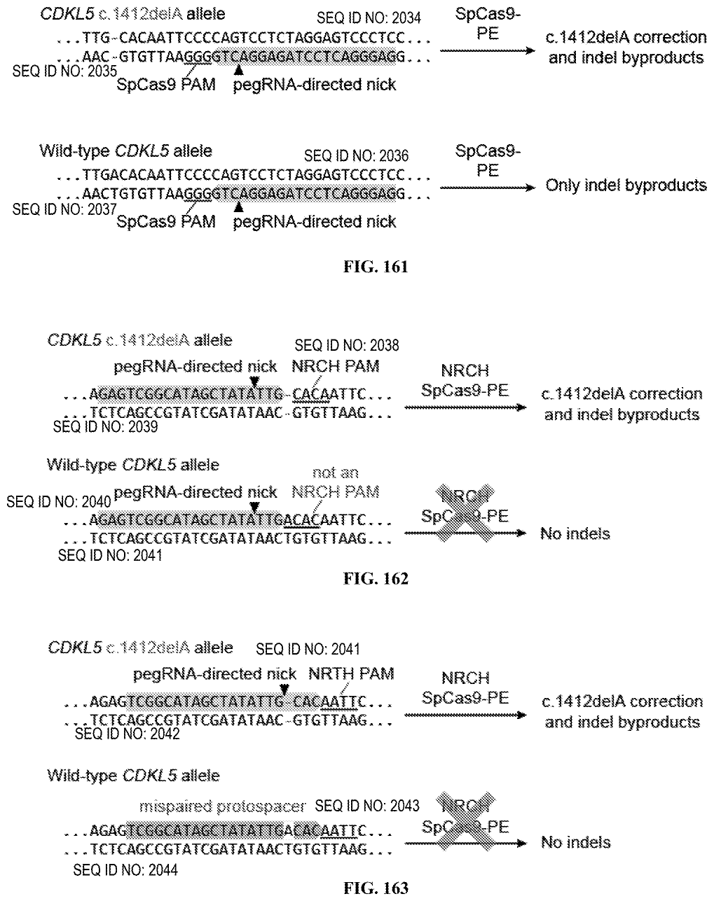

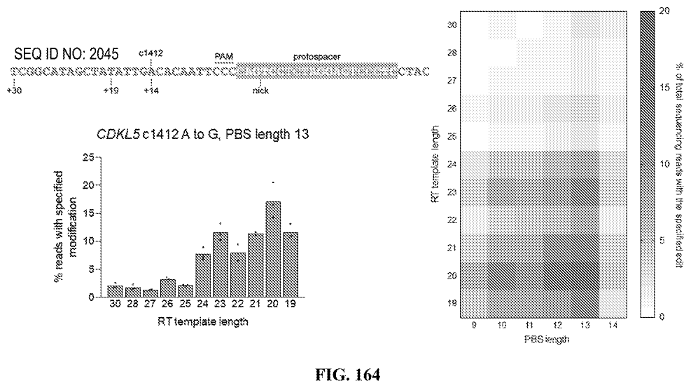

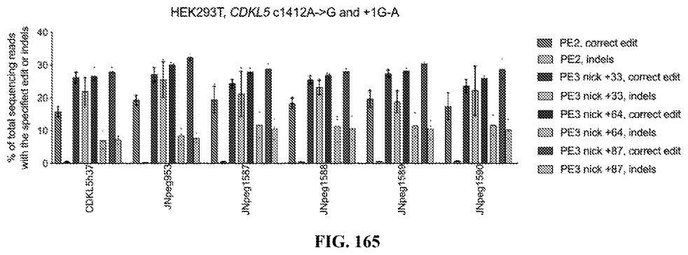

The embodiment of FIG. 3E provides the structure of another pegRNA contemplated herein. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a optional homology arm, a DNA synthesis template, and a primer binding site (PBS). In addition, the pegRNA may comprise an optional 3′ end modifier region (e1) and an optional 5′ end modifier region (e2). Still further, the pegRNA may comprise a transcriptional termination signal on the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The pegRNA could comprise, in certain embodiments, secondary RNA structures, such as, but not limited to, hairpins, stem/loops, toe loops, RNA-binding protein recruitment domains (e.g., the MS2 aptamer which recruits and binds to the MS2cp protein). These secondary structures could be positioned anywhere in the pegRNA molecule. For instance, such secondary structures could be position within the spacer, the gRNA core, or the extension arm, and in particular, within the e1 and/or e2 modifier regions. In addition to secondary RNA structures, the pegRNAs could comprise (e.g., within the e1 and/or e2 modifier regions) a chemical linker or a poly(N) linker or tail, where “N” can be any nucleobase. In some embodiments (e.g., as shown in FIG. 72(c)), the chemical linker may function to prevent reverse transcription of the sgRNA scaffold or core. In addition, in certain embodiments (e.g., see FIG. 72(c)), the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the pegRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the pegRNA (e.g., in the e1 or e2 region, or elsewhere and an MS2cp protein fused to the DNA polymerase, thereby co-localizing the DNA polymerase to the pegRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

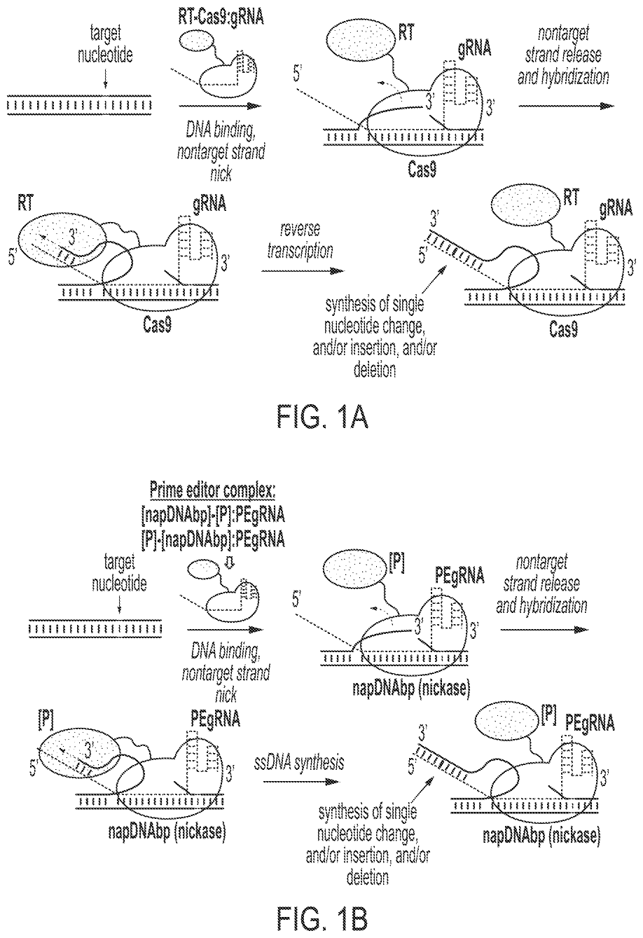

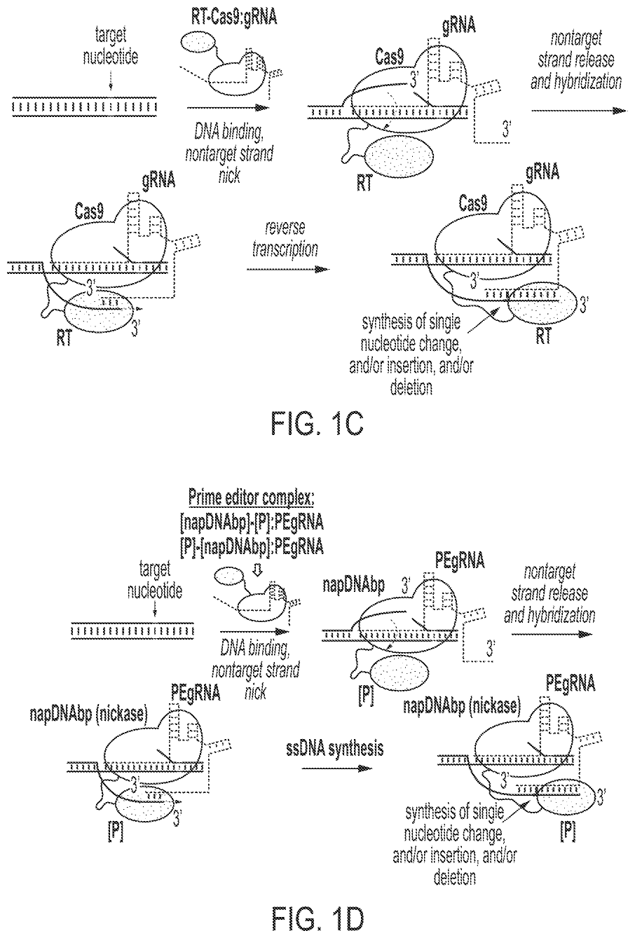

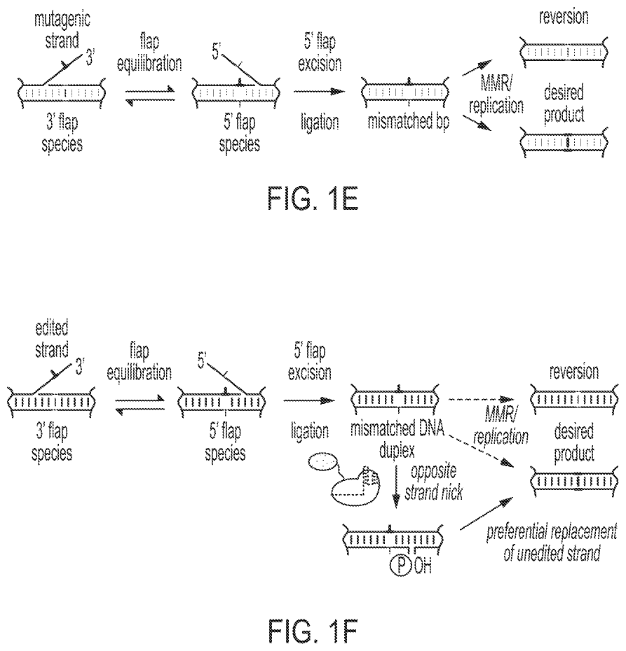

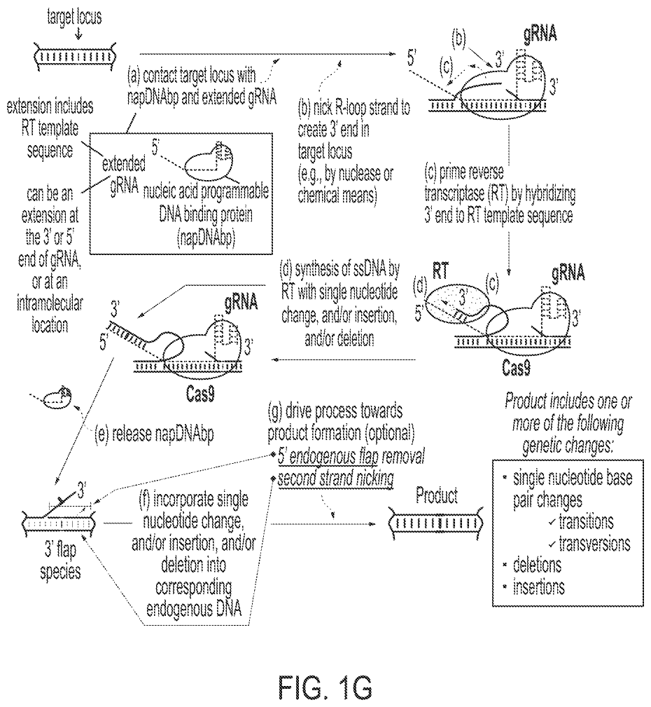

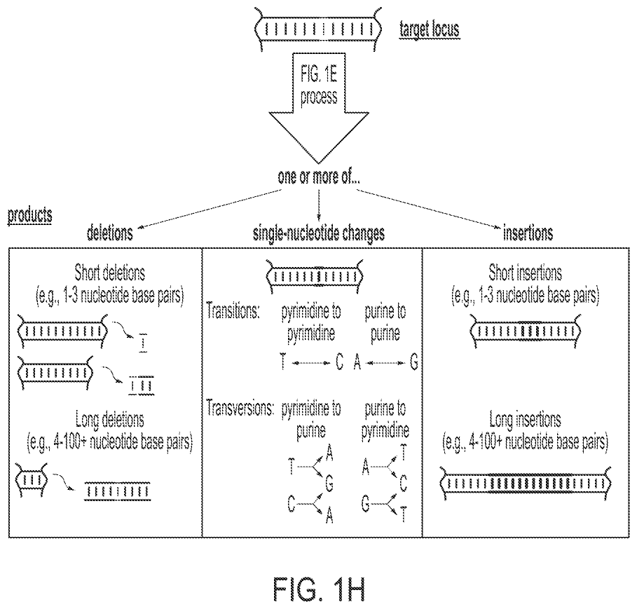

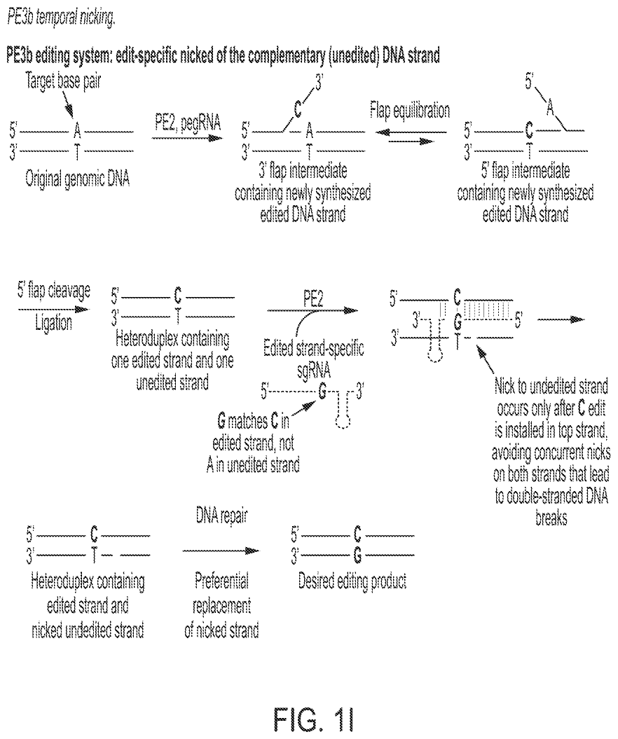

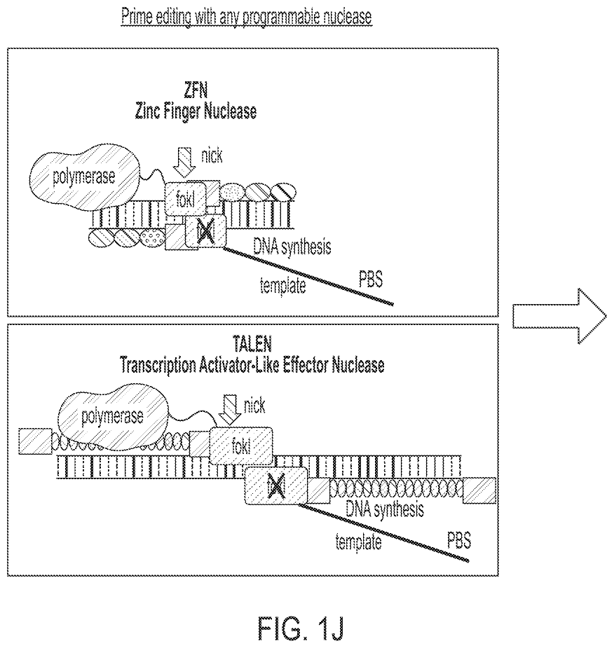

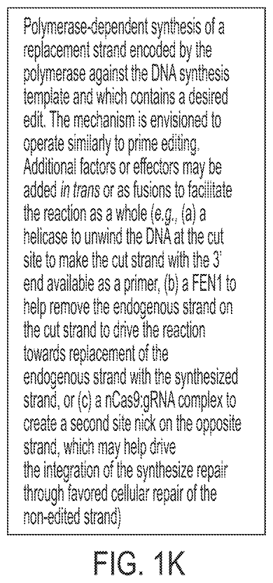

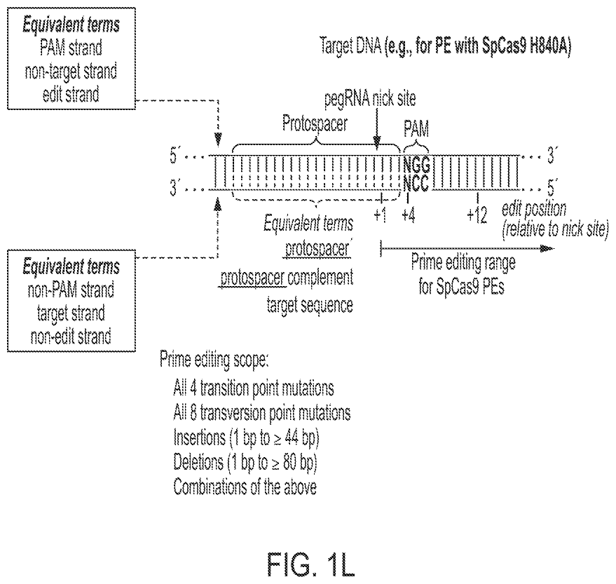

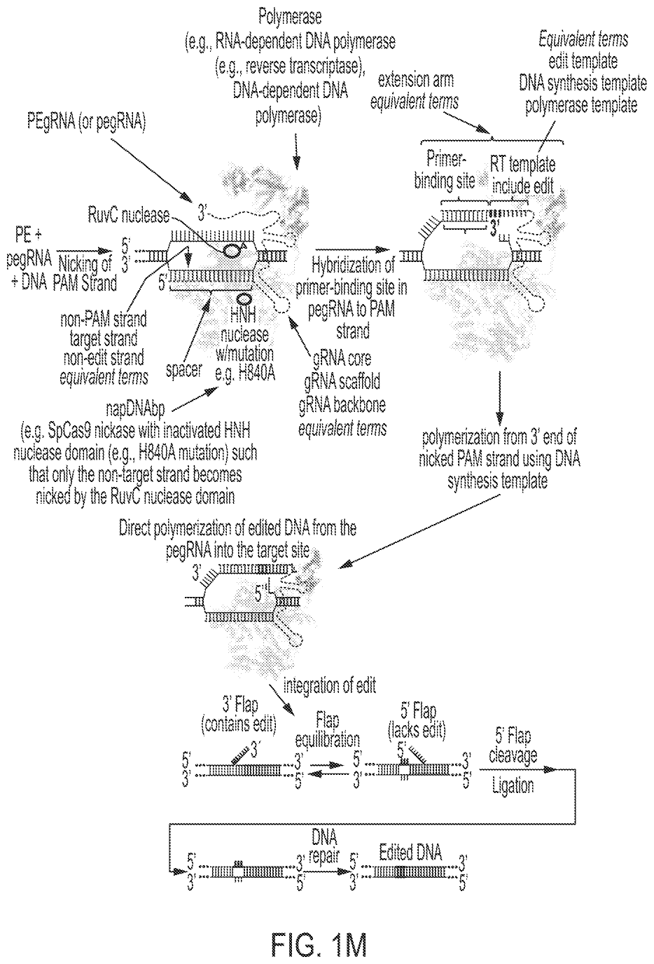

The schematic of FIG. 3F depicts the interaction of a typical pegRNA with a target site of a double stranded DNA and the concomitant production of a 3′ single stranded DNA flap containing the genetic change of interest. The double strand DNA is shown with the top strand (i.e., the target strand) in the 3′ to 5′ orientation and the lower strand (i.e., the PAM strand or non-target strand) in the 5′ to 3′ direction. The top strand comprises the complement of the “protospacer” and the complement of the PAM sequence and is referred to as the “target strand” because it is the strand that is target by and anneals to the spacer of the pegRNA. The complementary lower strand is referred to as the “non-target strand” or the “PAM strand” or the “protospacer strand” since it contains the PAM sequence (e.g., NGG) and the protospacer. Although not shown, the pegRNA depicted would be complexed with a Cas9 or equivalent domain of a prime editor. As shown in the schematic (FIG. 3F), the spacer sequence of the pegRNA anneals to the complementary region of the protospacer on the target strand. This interaction forms as DNA/RNA hybrid between the spacer RNA and the complement of the protospacer DNA, and induces the formation of an R loop in the protospacer. As taught elsewhere herein, the Cas9 protein (not shown) then induces a nick in the non-target strand, as shown. This then leads to the formation of the 3′ ssDNA flap region immediately upstream of the nick site which, in accordance with *z*, interacts with the 3′ end of the pegRNA at the primer binding site. The 3′ end of the ssDNA flap (i.e., the reverse transcriptase primer sequence) anneals to the primer binding site (A) on the pegRNA, thereby priming reverse transcriptase. Next, reverse transcriptase (e.g., provided in trans or provided cis as a fusion protein, attached to the Cas9 construct) then polymerizes a single strand of DNA which is coded for by the DNA synthesis template (including the edit template (B) and homology arm (C)). The polymerization continues towards the 5′ end of the extension arm. The polymerized strand of ssDNA forms a ssDNA 3′ end flap which, as describe elsewhere (e.g., as shown in FIG. 1G), invades the endogenous DNA, displacing the corresponding endogenous strand (which is removed as a 5′ ended DNA flap of endogenous DNA), and installing the desired nucleotide edit (single nucleotide base pair change, deletions, insertions (including whole genes) through DNA repair/replication rounds.

FIG. 3G depicts yet another embodiment of prime editing contemplated herein. In particular, the top schematic depicts one embodiment of a prime editor (PE), which comprises a fusion protein of a napDNAbp (e.g., SpCas9) and a polymerase (e.g., a reverse transcriptase), which are joined by a linker. The PE forms a complex with a pegRNA by binding to the gRNA core of the pegRNA. In the embodiment shown, the pegRNA is equipped with a 3′ extension arm that comprises, beginning at the 3′ end, a primer binding site (PBS) followed by a DNA synthesis template. The bottom schematic depicts a variant of a prime editor, referred to as a “trans prime editor (tPE).” In this embodiment, the DNA synthesis template and PBS are decoupled from the pegRNA and presented on a separate molecule, referred to as a trans prime editor RNA template (“tPERT”), which comprises an RNA-protein recruitment domain (e.g., a MS2 hairpin). The PE itself is further modified to comprise a fusion to a rPERT recruiting protein (“RP”), which is a protein which specifically recognizes and binds to the RNA-protein recruitment domain. In the example where the RNA-protein recruitment domain is an MS2 hairpin, the corresponding rPERT recruiting protein can be MS2cp of the MS2 tagging system. The MS2 tagging system is based on the natural interaction of the MS2 bacteriophage coat protein (“MCP” or “MS2cp”) with a stem-loop or hairpin structure present in the genome of the phage, i.e., the “MS2 hairpin” or “MS2 aptamer.” In the case of trans prime editing, the RP-PE:gRNA complex “recruits” a tPERT having the appropriate RNA-protein recruitment domain to co-localize with the PE:gRNA complex, thereby providing the PBS and DNA synthesis template in trans for use in prime editing, as shown in the example depicted in FIG. 3H.

FIG. 3H depicts the process of trans prime editing. In this embodiment, the trans prime editor comprises a “PE2” prime editor (i.e., a fusion of a Cas9(H840A) and a variant MMLV RT) fused to an MS2cp protein (i.e., a type of recruiting protein that recognizes and binds to an MS2 aptamer) and which is complexed with an sgRNA (i.e., a standard guide RNA as opposed to a pegRNA). The trans prime editor binds to the target DNA and nicks the nontarget strand. The MS2cp protein recruits a tPERT in trans through the specific interaction with the RNA-protein recruitment domain on the tPERT molecule. The tPERT becomes co-localized with the trans prime editor, thereby providing the PBS and DNA synthesis template functions in trans for use by the reverse transcriptase polymerase to synthesize a single strand DNA flap having a 3′ end and containing the desired genetic information encoded by the DNA synthesis template.

FIGS. 4A-4E demonstrate in vitro prime editing assays. FIG. 4A is a schematic of fluorescently labeled DNA substrates gRNA templated extension by an RT enzyme, PAGE. FIG. 4B shows prime editing with pre-nicked substrates, dCas9, and 5′-extended pegRNAs of differing synthesis template length. FIG. 4C shows the RT reaction with pre-nicked DNA substrates in the absence of Cas9. FIG. 4D shows prime editing on full dsDNA substrates with Cas9(H840A) and 5′-extended pegRNAs. FIG. 4E shows a 3′-extended pegRNAs template with pre-nicked and full dsDNA substrates. All reactions are with M-MLV RT.

FIG. 5 shows in vitro validations using 5′-extended pegRNAs with varying length synthesis templates. Fluorescently labeled (Cy5) DNA targets were used as substrates, and were pre-nicked in this set of experiments. The Cas9 used in these experiments is catalytically dead Cas9 (dCas9), and the RT used is Superscript III, a commercial RT derived from the Moloney-Murine Leukemia Virus (M-MLV). dCas9:gRNA complexes were formed from purified components. Then, the fluorescently labeled DNA substrate was added along with dNTPs and the RT enzyme. After 1 hour of incubation at 37° C., the reaction products were analyzed by denaturing urea-polyacrylamide gel electrophoresis (PAGE). The gel image shows extension of the original DNA strand to lengths that are consistent with the length of the reverse transcription template.

FIG. 6 shows in vitro validations using 5′-pegRNAs with varying length synthesis templates, which closely parallels those shown in FIG. 5. However, the DNA substrates are not pre-nicked in this set of experiments. The Cas9 used in these experiments is a Cas9 nickase (SpyCas9 H840A mutant) and the RT used is Superscript III, a commercial RT derived from the Moloney-Murine Leukemia Virus (M-MLV). The reaction products were analyzed by denaturing urea-polyacrylamide gel electrophoresis (PAGE). As shown in the gel, the nickase efficiently cleaves the DNA strand when the standard gRNA is used (gRNA_0, lane 3).

FIG. 7 demonstrates that 3′ extensions support DNA synthesis and do not significantly affect Cas9 nickase activity. Pre-nicked substrates (black arrow) are near-quantitatively converted to RT products when either dCas9 or Cas9 nickase is used (lanes 4 and 5). Greater than 50% conversion to the RT product (red arrow) is observed with full substrates (lane 3). Cas9 nickase (SpyCas9 H840A mutant), catalytically dead Cas9 (dCas9) and Superscript III, a commercial RT derived from the Moloney-Murine Leukemia Virus (M-MLV) are used.

FIG. 8 demonstrates dual color experiments that were used to determine if the RT reaction preferentially occurs with the gRNA in cis (bound in the same complex). Two separate experiments were conducted for 5′-extended and 3′-extended pegRNAs. Products were analyzed by PAGE. Product ratio calculated as (Cy3cis/Cy3trans)/(Cy5trans/Cy5cis).

FIGS. 9A-9D demonstrates a flap model substrate. FIG. 9A shows a dual-FP reporter for flap-directed mutagenesis. FIG. 9B shows stop codon repair in HEK cells. FIG. 9C shows sequenced yeast clones after flap repair. FIG. 9D shows testing of different flap features in human cells.

FIG. 10 demonstrates prime editing on plasmid substrates. A dual-fluorescent reporter plasmid was constructed for yeast (S. cerevisiae) expression. Expression of this construct in yeast produces only GFP. The in vitro prime editing reaction introduces a point mutation, and transforms the parent plasmid or an in vitro Cas9(H840A) nicked plasmid into yeast. The colonies are visualized by fluorescence imaging. Yeast dual-FP plasmid transformants are shown. Transforming the parent plasmid or an in vitro Cas9(H840A) nicked plasmid results in only green GFP expressing colonies. The prime editing reaction with 5′-extended or 3′-extended pegRNAs produces a mix of green and yellow colonies. The latter express both GFP and mCherry. More yellow colonies are observed with the 3′-extended pegRNA. A positive control that contains no stop codon is shown as well.

FIG. 11 shows prime editing on plasmid substrates similar to the experiment in FIG. 10, but instead of installing a point mutation in the stop codon, prime editing installs a single nucleotide insertion (left) or deletion (right) that repairs a frameshift mutation and allows for synthesis of downstream mCherry. Both experiments used 3′ extended pegRNAs.

FIG. 12 shows editing products of prime editing on plasmid substrates, characterized by Sanger sequencing. Individually colonies from the TRT transformations were selected and analyzed by Sanger sequencing. Precise edits were observed by sequencing select colonies. Green colonies contained plasmids with the original DNA sequence, while yellow colonies contained the precise mutation designed by the prime editing gRNA. No other point mutations or indels were observed.

FIG. 13 shows the potential scope for the new prime editing technology is shown and compared to deaminase-mediated base editor technologies.

FIG. 14 shows a schematic of editing in human cells.

FIG. 15 demonstrates the extension of the primer binding site in gRNA.

FIG. 16 shows truncated gRNAs for adjacent targeting.

FIGS. 17A-17C are graphs displaying the % T to A conversion at the target nucleotide after transfection of components in human embryonic kidney (HEK) cells. FIG. 17A shows data, which presents results using an N-terminal fusion of wild type MLV reverse transcriptase to Cas9(H840A) nickase (32-amino acid linker). FIG. 17B is similar to FIG. 17A, but for C-terminal fusion of the RT enzyme. FIG. 17C is similar to FIG. 17A but the linker between the MLV RT and Cas9 is 60 amino acids long instead of 32 amino acids.

FIG. 18 shows high purity T to A editing at HEK3 site by high-throughput amplicon sequencing. The output of sequencing analysis displays the most abundant genotypes of edited cells.

FIG. 19 shows editing efficiency at the target nucleotide (blue bars) alongside indel rates (orange bars). WT refers to the wild type MLV RT enzyme. The mutant enzymes (M1 through M4) contain the mutations listed to the right. Editing rates were quantified by high throughput sequencing of genomic DNA amplicons.

FIG. 20 shows editing efficiency of the target nucleotide when a single strand nick is introduced in the complementary DNA strand in proximity to the target nucleotide. Nicking at various distances from the target nucleotide was tested (triangles). Editing efficiency at the target base pair (blue bars) is shown alongside the indel formation rate (orange bars). The “none” example does not contain a complementary strand nicking guide RNA. Editing rates were quantified by high throughput sequencing of genomic DNA amplicons.

FIG. 21 demonstrates processed high throughput sequencing data showing the desired T to A transversion mutation and general absence of other major genome editing byproducts.

FIG. 22 provides a schematic of an exemplary process for conducting targeted mutagenesis with an error-prone reverse transcriptase on a target locus using a nucleic acid programmable DNA binding protein (napDNAbp) complexed with an pegRNA, i.e., prime editing with an error-prone RT. This process may be referred to as an embodiment of prime editing for targeted mutagenesis. The pegRNA comprises an extension at the 3′ or 5′ end of the guide RNA, or at an intramolecular location in the guide RNA. In step (a), the napDNAbp/gRNA complex contacts the DNA molecule and the gRNA guides the napDNAbp to bind to the target locus to be mutagenized. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence. In step (c), the 3′ end DNA strand interacts with the extended portion of the guide RNA in order to prime reverse transcription. In certain embodiments, the 3′ ended DNA strand hybridizes to a specific RT priming sequence on the extended portion of the guide RNA. In step (d), an error-prone reverse transcriptase is introduced which synthesizes a mutagenized single strand of DNA from the 3′ end of the primed site towards the 3′ end of the guide RNA. Exemplary mutations are indicated with an asterisk “*”. This forms a single-strand DNA flap comprising the desired mutagenized region. In step (e), the napDNAbp and guide RNA are released. Steps (f) and (g) relate to the resolution of the single strand DNA flap (comprising the mutagenized region) such that the desired mutagenized region becomes incorporated into the target locus. This process can be driven towards the desired product formation by removing the corresponding 5′ endogenous DNA flap that forms once the 3′ single strand DNA flap invades and hybridizes to the complementary sequence on the other strand. The process can also be driven towards product formation with second strand nicking, as exemplified in FIG. 1F. Following endogenous DNA repair and/or replication processes, the mutagenized region becomes incorporated into both strands of DNA of the DNA locus.

FIG. 23 is a schematic of gRNA design for contracting trinucleotide repeat sequences and trinucleotide repeat contraction with prime editing. Trinucleotide repeat expansion is associated with a number of human diseases, including Huntington's disease, Fragile X syndrome, and Friedreich's ataxia. The most common trinucleotide repeat contains CAG triplets, though GAA triplets (Friedreich's ataxia) and CGG triplets (Fragile X syndrome) also occur. Inheriting a predisposition to expansion, or acquiring an already expanded parental allele, increases the likelihood of acquiring the disease. Pathogenic expansions of trinucleotide repeats could hypothetically be corrected using prime editing. A region upstream of the repeat region can be nicked by an RNA-guided nuclease, then used to prime synthesis of a new DNA strand that contains a healthy number of repeats (which depends on the particular gene and disease). After the repeat sequence, a short stretch of homology is added that matches the identity of the sequence adjacent to the other end of the repeat (red strand). Invasion of the newly synthesized strand, and subsequent replacement of the endogenous DNA with the newly synthesized flap, leads to a contracted repeat allele.

FIG. 24 is a schematic showing precise 10-nucleotide deletion with prime editing. A guide RNA targeting the HEK3 locus was designed with a reverse transcription template that encodes a 10-nucleotide deletion after the nick site. Editing efficiency in transfected HEK cells was assessed using amplicon sequencing.

FIG. 25 is a schematic showing gRNA design for peptide tagging genes at endogenous genomic loci and peptide tagging with prime editing. The FlAsH and ReAsH tagging systems comprise two parts: (1) a fluorophore-biarsenical probe, and (2) a genetically encoded peptide containing a tetracysteine motif, exemplified by the sequence FLNCCPGCCMEP (SEQ ID NO: 1). When expressed within cells, proteins containing the tetracysteine motif can be fluorescently labeled with fluorophore-arsenic probes (see ref: J. Am. Chem. Soc., 2002, 124 (21), pp 6063-6076. DOI: 10.1021/ja017687n). The “sortagging” system employs bacterial sortase enzymes that covalently conjugate labeled peptide probes to proteins containing suitable peptide substrates (see ref: Nat. Chem. Biol. 2007 November; 3(11):707-8. DOI: 10.1038/nchembio.2007.31). The FLAG-tag (DYKDDDDK (SEQ ID NO: 2)), V5-tag (GKPIPNPLLGLDST (SEQ ID NO: 3)), GCN4-tag (EELLSKNYHLENEVARLKK (SEQ ID NO: 4)), HA-tag (YPYDVPDYA (SEQ ID NO: 5)), and Myc-tag (EQKLISEEDL (SEQ ID NO: 6)) are commonly employed as epitope tags for immunoassays. The pi-clamp encodes a peptide sequence (FCPF (SEQ ID NO: 7)) that can by labeled with a pentafluoro-aromatic substrates (ref: Nat. Chem. 2016 February; 8(2):120-8. doi: 10.1038/nchem.2413).

FIG. 26A shows precise installation of a His6-tag and a FLAG-tag into genomic DNA. A guide RNA targeting the HEK3 locus was designed with a reverse transcription template that encodes either an 18-nt His-tag insertion or a 24-nt FLAG-tag insertion. Editing efficiency in transfected HEK cells was assessed using amplicon sequencing. Note that the full 24-nt sequence of the FLAG-tag is outside of the viewing frame (sequencing confirmed full and precise insertion). FIG. 26B shows a schematic outlining various applications involving protein/peptide tagging, including (a) rendering proteins soluble or insoluble, (b) changing or tracking the cellular localization of a protein, (c) extending the half-life of a protein, (d) facilitating protein purification, and (e) facilitating the detection of proteins.

FIG. 27 shows an overview of prime editing by installing a protective mutation in PRNP that prevents or halts the progression of prion disease. The pegRNA sequences correspond to residues 1-20 of SEQ ID NO: 810 on the left (i.e., 5′ of the sgRNA scaffold) and residues 21-43 of SEQ ID NO 810 on the right (i.e., 3′ of the sgRNA scaffold).

FIG. 28A is a schematic of PE-based insertion of sequences encoding RNA motifs. FIG. 28B is a list (not exhaustive) of some example motifs that could potentially be inserted, and their functions.

FIG. 29A is a depiction of a prime editor. FIG. 29B shows possible modifications to genomic, plasmid, or viral DNA directed by a PE. FIG. 29C shows an example scheme for insertion of a library of peptide loops into a specified protein (in this case GFP) via a library of pegRNAs. FIG. 29D shows an example of possible programmable deletions of codons or N-, or C-terminal truncations of a protein using different pegRNAs. Deletions would be predicted to occur with minimal generation of frameshift mutations.

FIG. 30 shows a possible scheme for iterative insertion of codons in a continual evolution system, such as PACE.

FIG. 31 is an illustration of an engineered gRNA showing the gRNA core, ˜20 nt spacer matching the sequence of the targeted gene, the reverse transcription template with immunogenic epitope nucleotide sequence and the primer binding site matching the sequence of the targeted gene.

FIG. 32 is a schematic showing using prime editing as a means to insert known immunogenicity epitopes into endogenous or foreign genomic DNA, resulting in modification of the corresponding proteins.

FIG. 33 is a schematic showing pegRNA design for primer binding sequence insertions and primer binding insertion into genomic DNA using prime editing for determining off-target editing. In this embodiment, prime editing is conducted inside a living cell, a tissue, or an animal model. As a first step, an appropriate pegRNA is designed. The top schematic shows an exemplary pegRNA that may be used in this aspect. The spacer sequence in the pegRNA (labeled “protospacer”) is complementary to one of the strands of the genomic target. The PE:pegRNA complex (i.e., the PE complex) installs a single stranded 3′ end flap at the nick site which contains the encoded primer binding sequence and the region of homology (coded by the homology arm of the pegRNA) that is complementary to the region just downstream of the cut site (in red). Through flap invasion and DNA repair/replication processes, the synthesized strand becomes incorporated into the DNA, thereby installing the primer binding site. This process can occur at the desired genomic target, but also at other genomic sites that might interact with the pegRNA in an off-target manner (i.e., the pegRNA guides the PE complex to other off-target sites due to the complementarity of the spacer region to other genomic sites that are not the intended genomic site). Thus, the primer binding sequence may be installed not only at the desired genomic target, but at off-target genomic sites elsewhere in the genome. In order to detect the insertion of these primer binding sites at both the intended genomic target sites and the off-target genomic sites, the genomic DNA (post-PE) can be isolated, fragmented, and ligated to adapter nucleotides (shown in red). Next, PCR may be carried out with PCR oligonucleotides that anneal to the adapters and to the inserted primer binding sequence to amplify on-target and off-target genomic DNA regions into which the primer binding site was inserted by PE. High throughput sequencing then may be conducted to and sequence alignments to identify the insertion points of PE-inserted primer binding sequences at either the on-target site or at off-target sites.

FIG. 34 is a schematic showing the precise insertion of a gene with PE.

FIG. 35A is a schematic showing the natural insulin signaling pathway. FIG. 35B is a schematic showing FKBP12-tagged insulin receptor activation controlled by FK1012.

FIG. 36 shows small-molecule monomers. References: bumped FK506 mimic (2)¹⁰⁷

FIG. 37 shows small-molecule dimers. References: FK1012 4⁹⁵⁻⁹⁶; FK1012 5¹⁰⁸; FK1012 6¹⁰⁷; AP1903 7¹⁰⁷; cyclosporin A dimer 8⁹⁸; FK506-cyclosporin A dimer (FkCsA) 9¹⁰⁰.

FIGS. 38A-38F provide an overview of prime editing and feasibility studies in vitro and in yeast cells. FIG. 38A shows the 75,122 known pathogenic human genetic variants in ClinVar (accessed July, 2019), classified by type. FIG. 38B shows that a prime editing complex consists of a prime editor (PE) protein containing an RNA-guided DNA-nicking domain, such as Cas9 nickase, fused to an engineered reverse transcriptase domain and complexed with a prime editing guide RNA (pegRNA). The PE:pegRNA complex binds the target DNA site and enables a large variety of precise DNA edits at a wide range of DNA positions before or after the target site's protospacer adjacent motif (PAM). FIG. 38C shows that upon DNA target binding, the PE:pegRNA complex nicks the PAM-containing DNA strand. The resulting free 3′ end hybridizes to the primer-binding site of the pegRNA. The reverse transcriptase domain catalyzes primer extension using the RT template of the pegRNA, resulting in a newly synthesized DNA strand containing the desired edit (the 3′ flap). Equilibration between the edited 3′ flap and the unedited 5′ flap containing the original DNA, followed by cellular 5′ flap cleavage and ligation, and DNA repair or replication to resolve the heteroduplex DNA, results in stably edited DNA. FIG. 38D shows in vitro 5′-extended pegRNA primer extension assays with pre-nicked dsDNA substrates containing 5′-Cy5 labeled PAM strands, dCas9, and a commercial M-MLV RT variant (RT, Superscript III). dCas9 was complexed with pegRNAs containing RT template of varying lengths, then added to DNA substrates along with the indicated components. Reactions were incubated at 37° C. for 1 hour, then analyzed by denaturing urea PAGE and visualized for Cy5 fluorescence. FIG. 38E shows primer extension assays performed as in FIG. 38D using 3′-extended pegRNAs pre-complexed with dCas9 or Cas9 H840A nickase, and pre-nicked or non-nicked 5′-Cy5-labeled dsDNA substrates. FIG. 38F shows yeast colonies transformed with GFP-mCherry fusion reporter plasmids edited in vitro with pegRNAs, Cas9 nickase, and RT. Plasmids containing nonsense or frameshift mutations between GFP and mCherry were edited with 5′-extended or 3′-extended pegRNAs that restore mCherry translation via transversion mutation, 1-bp insertion, or 1-bp deletion. GFP and mCherry double-positive cells (yellow) reflect successful editing.

FIGS. 39A-39D show prime editing of genomic DNA in human cells by PE1 and PE2. FIG. 39A shows pegRNAs contain a spacer sequence, a sgRNA scaffold, and a 3′ extension containing a primer-binding site (green) and a reverse transcription (RT) template (purple), which contains the edited base(s) (red). The primer-binding site hybridizes to the PAM-containing DNA strand immediately upstream of the site of nicking. The RT template is homologous to the DNA sequence downstream of the nick, with the exception of the encoded edit. FIG. 39B shows an installation of a T•A-to-A•T transversion edit at the HEK3 site in HEK293T cells using Cas9 H840A nickase fused to wild-type M-MLV reverse transcriptase (PE1) and pegRNAs of varying primer-binding site lengths. FIG. 39C shows the use of an engineered pentamutant M-MLV reverse transcriptase (D200N, L603W, T306K, W313F, T330P) in PE2 substantially improves prime editing transversion efficiencies at five genomic sites in HEK293T cells, and small insertion and small deletion edits at HEK3. FIG. 39D is a comparison of PE2 editing efficiencies with varying RT template lengths at five genomic sites in HEK293T cells. Values and error bars reflect the mean and s.d. of three independent biological replicates.

FIGS. 40A-40C show PE3 and PE3b systems nick the non-edited strand to increase prime editing efficiency. FIG. 40A is an overview of the prime editing by PE3. After initial synthesis of the edited strand, DNA repair will remove either the newly synthesized strand containing the edit (3′ flap excision) or the original genomic DNA strand (5′ flap excision). 5′ flap excision leaves behind a DNA heteroduplex containing one edited strand and one non-edited strand. Mismatch repair machinery or DNA replication could resolve the heteroduplex to give either edited or non-edited products. Nicking the non-edited strand favors repair of that strand, resulting in preferential generation of stable duplex DNA containing the desired edit. FIG. 40B shows the effect of complementary strand nicking on PE3-mediated prime editing efficiency and indel formation. “None” refers to PE2 controls, which do not nick the complementary strand. FIG. 40C is a comparison of editing efficiencies with PE2 (no complementary strand nick), PE3 (general complementary strand nick), and PE3b (edit-specific complementary strand nick). All editing yields reflect the percentage of total sequencing reads that contain the intended edit and do not contain indels among all treated cells, with no sorting. Values and error bars reflect the mean and s.d. of three independent biological replicates.

FIGS. 41A-41K show targeted insertions, deletions, and all 12 types of point mutations with PE3 at seven endogenous human genomic loci in HEK293T cells. FIG. 41A is a graph showing all 12 types of single-nucleotide transition and transversion edits from position +1 to +8 (counting the location of the pegRNA-induced nick as between position +1 and −1) of the HEK3 site using a 10-nt RT template. FIG. 41B is a graph showing long-range PE3 transversion edits at the HEK3 site using a 34-nt RT template. FIGS. 41C-41H are graphs showing all 12 types of transition and transversion edits at various positions in the prime editing window for (FIG. 41C) RNF2, (FIG. 41D) FANCF, (FIG. 41E) EMX1, (FIG. 41F) RUNX1, (FIG. 41G) VEGFA, and (FIG. 41H) DNMT1. FIG. 41I is a graph showing targeted 1- and 3-bp insertions, and 1- and 3-bp deletions with PE3 at seven endogenous genomic loci. FIG. 41J is a graph showing the targeted precise deletions of 5 to 80 bp at the HEK3 target site. FIG. 41K is a graph showing a combination edits of insertions and deletions, insertions and point mutations, deletions and point mutations, and double point mutations at three endogenous genomic loci. All editing yields reflect the percentage of total sequencing reads that contain the intended edit and do not contain indels among all treated cells, with no sorting. Values and error bars reflect the mean and s.d. of three independent biological replicates.

FIGS. 42A-42H show the comparison of prime editing and base editing, and off-target editing by Cas9 and PE3 at known Cas9 off-target sites. FIG. 42A shows total C•G-to-T•A editing efficiency at the same target nucleotides for PE2, PE3, BE2max, and BE4max at endogenous HEK3, FANCF, and EMX1 sites in HEK293T cells. FIG. 42B shows indel frequency from treatments in FIG. 42A. FIG. 42C shows the editing efficiency of precise C•G-to-T•A edits (without bystander edits or indels) for PE2, PE3, BE2max, and BE4max at HEK3, FANCF, and EMX1. For EMX1, precise PE combination edits of all possible combinations of C•G-to-T•A conversion at the three targeted nucleotides are also shown.

FIG. 42D shows the total A•T-to-G•C editing efficiency for PE2, PE3, ABEdmax, and ABEmax at HEK3 and FANCF. FIG. 42E shows the precise A•T-to-G•C editing efficiency without bystander edits or indels for at HEK3 and FANCF. FIG. 42F shows indel frequency from treatments in FIG. 42D. FIG. 42G shows the average triplicate editing efficiencies (percentage sequencing reads with indels) in HEK293T cells for Cas9 nuclease at four on-target and 16 known off-target sites. The 16 off-target sites examined were the top four previously reported off-target sites^{118, 159} for each of the four on-target sites. For each on-target site, Cas9 was paired with a sgRNA or with each of four pegRNAs that recognize the same protospacer. FIG. 42H shows the average triplicate on-target and off-target editing efficiencies and indel efficiencies (below in parentheses) in HEK293T cells for PE2 or PE3 paired with each pegRNA in (FIG. 42G). On-target editing yields reflect the percentage of total sequencing reads that contain the intended edit and do not contain indels among all treated cells, with no sorting. Off-target editing yields reflect off-target locus modification consistent with prime editing. Values and error bars reflect the mean and s.d. of three independent biological replicates.

FIGS. 43A-43I show prime editing in various human cell lines and primary mouse cortical neurons, installation and correction of pathogenic transversion, insertion, or deletion mutations, and comparison of prime editing and HDR. FIG. 43A is a graph showing the installation (via T•A-to-A•T transversion) and correction (via A•T-to-T•A transversion) of the pathogenic E6V mutation in HBB in HEK293T cells. Correction either to wild-type HBB, or to HBB containing a silent mutation that disrupts the pegRNA PAM, is shown. FIG. 43B is a graph showing the installation (via 4-bp insertion) and correction (via 4-bp deletion) of the pathogenic HEXA 1278+TATC allele in HEK293T cells. Correction either to wild-type HEXA, or to HEXA containing a silent mutation that disrupts the pegRNA PAM, is shown. FIG. 43C is a graph showing the installation of the protective G127V variant in PRNP in HEK293T cells via G•C-to-T•A transversion. FIG. 43D is a graph showing prime editing in other human cell lines including K562 (leukemic bone marrow cells), U2OS (osteosarcoma cells), and HeLa (cervical cancer cells). FIG. 43E is a graph showing the installation of a G•C-to-T•A transversion mutation in DNMT1 of mouse primary cortical neurons using a dual split-intein PE3 lentivirus system, in which the N-terminal half is Cas9 (1-573) fused to N-intein and through a P2A self-cleaving peptide to GFP-KASH, and the C-terminal half is the C-intein fused to the remainder of PE2. PE2 halves are expressed from a human synapsin promoter that is highly specific for mature neurons. Sorted values reflect editing or indels from GFP-positive nuclei, while unsorted values are from all nuclei. FIG. 43F is a comparison of PE3 and Cas9-mediated HDR editing efficiencies at endogenous genomic loci in HEK293T cells. FIG. 43G is a comparison of PE3 and Cas9-mediated HDR editing efficiencies at endogenous genomic loci in K562, U2OS, and HeLa cells. FIG. 43H is a comparison of PE3 and Cas9-mediated HDR indel byproduct generation in HEK293T, K562, U2OS, and HeLa cells. FIG. 43I shows targeted insertion of a His6 tag (18 bp), FLAG epitope tag (24 bp), or extended LoxP site (44 bp) in HEK293T cells by PE3. All editing yields reflect the percentage of total sequencing reads that contain the intended edit and do not contain indels among all treated cells. Values and error bars reflect the mean and s.d. of three independent biological replicates.

FIGS. 44A-44G show in vitro prime editing validation studies with fluorescently labeled DNA substrates. FIG. 44A shows electrophoretic mobility shift assays with dCas9, 5′-extended pegRNAs and 5′-Cy5-labeled DNA substrates. pegRNAs 1 through 5 contain a 15-nt linker sequence (linker A for pegRNA 1, linker B for pegRNAs 2 through 5) between the spacer and the PBS, a 5-nt PBS sequence, and RT templates of 7 nt (pegRNAs 1 and 2), 8 nt (pegRNA 3), 15 nt (pegRNA 4), and 22 nt (pegRNA 5). pegRNAs are those used in FIGS. 44E and 44F; full sequences are listed in Tables 2A-2C. FIG. 44B shows in vitro nicking assays of Cas9 H840A using 5′-extended and 3′-extended pegRNAs. FIG. 44C shows Cas9-mediated indel formation in HEK293T cells at HEK3 using 5′-extended and 3′-extended pegRNAs. FIG. 44D shows an overview of prime editing in vitro biochemical assays. 5′-Cy5-labeled pre-nicked and non-nicked dsDNA substrates were tested. sgRNAs, 5′-extended pegRNAs, or 3′-extended pegRNAs were pre-complexed with dCas9 or Cas9 H840A nickase, then combined with dsDNA substrate, M-MLV RT, and dNTPs. Reactions were allowed to proceed at 37° C. for 1 hour prior to separation by denaturing urea PAGE and visualization by Cy5 fluorescence. FIG. 44E shows primer extension reactions using 5′-extended pegRNAs, pre-nicked DNA substrates, and dCas9 lead to significant conversion to RT products. FIG. 44F shows primer extension reactions using 5′-extended pegRNAs as in FIG. 44B, with non-nicked DNA substrate and Cas9 H840A nickase. Product yields are greatly reduced by comparison to pre-nicked substrate. FIG. 44G shows an in vitro primer extension reaction using a 3′-pegRNA generates a single apparent product by denaturing urea PAGE. The RT product band was excised, eluted from the gel, then subjected to homopolymer tailing with terminal transferase (TdT) using either dGTP or dATP. Tailed products were extended by poly-T or poly-C primers, and the resulting DNA was sequenced. Sanger traces indicate that three nucleotides derived from the gRNA scaffold were reverse transcribed (added as the final 3′ nucleotides to the DNA product). Note that in mammalian cell prime editing experiments, pegRNA scaffold insertion is much rarer than in vitro (FIGS. 56A-56D), potentially due to the inability of the tethered reverse transcriptase to access the Cas9-bound guide RNA scaffold, and/or cellular excision of mismatched 3′ ends of 3′ flaps containing pegRNA scaffold sequences.

FIGS. 45A-45G show cellular repair in yeast of 3′ DNA flaps from in vitro prime editing reactions. FIG. 45A shows that dual fluorescent protein reporter plasmids contain GFP and mCherry open reading frames separated by a target site encoding an in-frame stop codon, a +1 frameshift, or a −1 frameshift. Prime editing reactions were carried out in vitro with Cas9 H840A nickase, pegRNA, dNTPs, and M-MLV reverse transcriptase, and then transformed into yeast. Colonies that contain unedited plasmids produce GFP but not mCherry. Yeast colonies containing edited plasmids produce both GFP and mCherry as a fusion protein. FIG. 45B shows an overlay of GFP and mCherry fluorescence for yeast colonies transformed with reporter plasmids containing a stop codon between GFP and mCherry (unedited negative control, top), or containing no stop codon or frameshift between GFP and mCherry (pre-edited positive control, bottom). FIGS. 45C-45F show a visualization of mCherry and GFP fluorescence from yeast colonies transformed with in vitro prime editing reaction products. FIG. 45C shows a stop codon correction via T•A-to-A•T transversion using a 3′-extended pegRNA, or a 5′-extended pegRNA, as shown in FIG. 45D. FIG. 45E shows a +1 frameshift correction via a 1-bp deletion using a 3′-extended pegRNA. FIG. 45F shows a −1 frameshift correction via a 1-bp insertion using a 3′-extended pegRNA. FIG. 45G shows Sanger DNA sequencing traces from plasmids isolated from GFP-only colonies in FIG. 45B and GFP and mCherry double-positive colonies in FIG. 45C.

FIGS. 46A-46F show correct editing versus indel generation with PE1. FIG. 46A shows T•A-to-A•T transversion editing efficiency and indel generation by PE1 at the +1 position of HEK3 using pegRNAs containing 10-nt RT templates and a PBS sequences ranging from 8-17 nt. FIG. 46B shows G•C-to-T•A transversion editing efficiency and indel generation by PE1 at the +5 position of EMX1 using pegRNAs containing 13-nt RT templates and a PBS sequences ranging from 9-17 nt. FIG. 46C shows G•C-to-T•A transversion editing efficiency and indel generation by PE1 at the +5 position of FANCF using pegRNAs containing 17-nt RT templates and a PBS sequences ranging from 8-17 nt. FIG. 46D shows C•G-to-A•T transversion editing efficiency and indel generation by PE1 at the +1 position of RNF2 using pegRNAs containing 11-nt RT templates and a PBS sequences ranging from 9-17 nt. FIG. 46E shows G•C-to-T•A transversion editing efficiency and indel generation by PE1 at the +2 position of HEK4 using pegRNAs containing 13-nt RT templates and a PBS sequences ranging from 7-15 nt. FIG. 46F shows PE1-mediated +1 T deletion, +1 A insertion, and +1 CTT insertion at the HEK3 site using a 13-nt PBS and 10-nt RT template. Sequences of pegRNAs are those used in FIG. 39C (see Tables 3A-3R). Values and error bars reflect the mean and s.d. of three independent biological replicates.

FIGS. 47A-47S show the evaluation of M-MLV RT variants for prime editing. FIG. 47A shows the abbreviations for prime editor variants used in this figure. FIG. 47B shows targeted insertion and deletion edits with PE1 at the HEK3 locus. FIGS. 47C-47H show a comparison of 18 prime editor constructs containing M-MLV RT variants for their ability to install a +2 G•C-to-C•G transversion edit at HEK3 as shown in FIG. 47C, a 24-bp FLAG insertion at HEK3 as shown in FIG. 47D, a +1 C•G-to-A•T transversion edit at RNF2 as shown in FIG. 47E, a +1 G•C-to-C•G transversion edit at EMX1 as shown in FIG. 47F, a +2 T•A-to-A•T transversion edit at HBB as shown in FIG. 47G, and a +1 G•C-to-C•G transversion edit at FANCF as shown in FIG. 47H. FIGS. 47I-47N show a comparison of four prime editor constructs containing M-MLV variants for their ability to install the edits shown in FIGS. 47C-47H in a second round of independent experiments. FIGS. 470-47S show PE2 editing efficiency at five genomic loci with varying PBS lengths. FIG. 47O shows a +1 T•A-to-A•T variation at HEK3. FIG. 47P shows a +5 G•C-to-T•A variation at EMX1. FIG. 47Q shows a +5 G•C-to-T•A variation at FANCF. FIG. 47R shows a +1 C•G-to-A•T variation at RNF2. FIG. 47S shows a +2 G•C-to-T•A variation at HEK4. Values and error bars reflect the mean and s.d. of three independent biological replicates.

FIGS. 48A-48C show design features of pegRNA PBS and RT template sequences. FIG. 48A shows PE2-mediated +5 G•C-to-T•A transversion editing efficiency (blue line) at VEGFA in HEK293T cells as a function of RT template length. Indels (gray line) are plotted for comparison. The sequence below the graph shows the last nucleotide templated for synthesis by the pegRNA. G nucleotides (templated by a C in the pegRNA) are highlighted; RT templates that end in C should be avoided during pegRNA design to maximize prime editing efficiencies. FIG. 48B shows +5 G•C-to-T•A transversion editing and indels for DNMT1 as in FIG. 48A. FIG. 48C shows +5 G•C-to-T•A transversion editing and indels for RUNX1 as in FIG. 48A. Values and error bars reflect the mean and s.d. of three independent biological replicates.

FIGS. 49A-49B show the effects of PE2, PE2 R110S K103L, Cas9 H840A nickase, and dCas9 on cell viability. HEK293T cells were transfected with plasmids encoding PE2, PE2 R110S K103L, Cas9 H840A nickase, or dCas9, together with a HEK3-targeting pegRNA plasmid. Cell viability was measured every 24 hours post-transfection for 3 days using the CellTiter-Glo 2.0 assay (Promega). FIG. 49A shows viability, as measured by luminescence, at 1, 2, or 3 days post-transfection. Values and error bars reflect the mean and s.e.m. of three independent biological replicates each performed in technical triplicate. FIG. 49B shows percent editing and indels for PE2, PE2 R110S K103L, Cas9 H840A nickase, or dCas9, together with a HEK3-targeting pegRNA plasmid that encodes a +5 G to A edit. Editing efficiencies were measured on day 3 post-transfection from cells treated alongside of those used for assaying viability in FIG. 49A. Values and error bars reflect the mean and s.d. of three independent biological replicates.

FIGS. 50A-50B show PE3-mediated HBB E6V correction and HEXA 1278+TATC correction by various pegRNAs. FIG. 50A shows a screen of 14 pegRNAs for correction of the HBB E6V allele in HEK293T cells with PE3. All pegRNAs evaluated convert the HBB E6V allele back to wild-type HBB without the introduction of any silent PAM mutation. FIG. 50B shows a screen of 41 pegRNAs for correction of the HEXA 1278+TATC allele in HEK293T cells with PE3 or PE3b. Those pegRNAs labeled HEXAs correct the pathogenic allele by a shifted 4-bp deletion that disrupts the PAM and leaves a silent mutation. Those pegRNAs labeled HEXA correct the pathogenic allele back to wild-type. Entries ending in “b” use an edit-specific nicking sgRNA in combination with the pegRNA (the PE3b system). Values and error bars reflect the mean and s.d. of three independent biological replicates.

FIGS. 51A-51F show a PE3 activity in human cell lines and a comparison of PE3 and Cas9-initiated HDR. Efficiency of generating the correct edit (without indels) and indel frequency for PE3 and Cas9-initiated HDR in HEK293T cells as shown in FIG. 51A, K562 cells as shown in FIG. 51B, U2OS cells as shown in FIG. 51C, and HeLa cells as shown in FIG. 51D. Each bracketed editing comparison installs identical edits with PE3 and Cas9-initiated HDR. Non-targeting controls are PE3 and a pegRNA that targets a non-target locus. FIG. 51E shows control experiments with non-targeting pegRNA+PE3, and with dCas9+sgRNA, compared with wild-type Cas9 HDR experiments confirming that ssDNA donor HDR template, a common contaminant that artificially elevates apparent HDR efficiencies, does not contribute to the HDR measurements in FIGS. 51A-51D. FIG. 51F shows example HEK3 site allele tables from genomic DNA samples isolated from K562 cells after editing with PE3 or with Cas9-initiated HDR. Alleles were sequenced on an Illumina MiSeq and analyzed with CRISPResso2¹⁷⁸. The reference HEK3 sequence from this region is at the top. Allele tables are shown for a non-targeting pegRNA negative control, a +1 CTT insertion at HEK3 using PE3, and a +1 CTT insertion at HEK3 using Cas9-initiated HDR. Allele frequencies and corresponding Illumina sequencing read counts are shown for each allele. All alleles observed with frequency ≥0.20% are shown. Values and error bars reflect the mean and s.d. of three independent biological replicates.

FIGS. 52A-52D show distribution by length of pathogenic insertions, duplications, deletions, and indels in the ClinVar database. The ClinVar variant summary was downloaded from NCBI Jul. 15, 2019. The lengths of reported insertions, deletions, and duplications were calculated using reference and alternate alleles, variant start and stop positions, or appropriate identifying information in the variant name. Variants that did not report any of the above information were excluded from the analysis. The lengths of reported indels (single variants that include both insertions and deletions relative to the reference genome) were calculated by determining the number of mismatches or gaps in the best pairwise alignment between the reference and alternate alleles.

FIGS. 53A-53B show FACS gating examples for GFP-positive cell sorting. Below are examples of original batch analysis files outlining the sorting strategy used for generating HEXA 1278+TATC and HBB E6V HEK293T cell lines. The image data was generated on a Sony LE-MA900 cytometer using Cell Sorter Software v. 3.0.5. Graphic 1 shows gating plots for cells that do not express GFP. Graphic 2 shows an example sort of P2A-GFP-expressing cells used for isolating the HBB E6V HEK293T cell lines. HEK293T cells were initially gated on population using FSC-A/BSC-A (Gate A), then sorted for singlets using FSC-A/FSC-H (Gate B). Live cells were sorted for by gating DAPI-negative cells (Gate C). Cells with GFP fluorescence levels that were above those of the negative-control cells were sorted for using EGFP as the fluorochrome (Gate D). FIG. 53A shows HEK293T cells (GFP-negative). FIG. 53B shows a representative plot of FACS gating for cells expressing PE2-P2A-GFP. FIG. 53C shows the genotypes for HEXA 1278+TATC homozygote HEK293T cells. FIG. 53D shows allele tables for HBB E6V homozygote HEK293T cell lines.

FIG. 54 is a schematic which summarizes the pegRNA cloning procedure.

FIGS. 55A-55G are schematics of pegRNA designs. FIG. 55A shows a simple diagram of pegRNA with domains labeled (left) and bound to nCas9 at a genomic site (right). FIG. 55B shows various types of modifications to pegRNA which can increase activity. FIG. 55C shows modifications to pegRNA to increase transcription of longer RNAs via promoter choice and 5′, 3′ processing and termination. FIG. 55D shows the lengthening of the P1 system, which is an example of a scaffold modification. FIG. 55E shows that the incorporation of synthetic modifications within the template region, or elsewhere within the pegRNA, could increase activity. FIG. 55F shows that a designed incorporation of minimal secondary structure within the template could prevent formation of longer, more inhibitory, secondary structure. FIG. 55G shows a split pegRNA with a second template sequence anchored by an RNA element at the 3′ end of the pegRNA (left). Incorporation of elements at the 5′ or 3′ ends of the pegRNA could enhance RT binding.

FIGS. 56A-56D show the incorporation of pegRNA scaffold sequence into target loci. HTS data were analyzed for pegRNA scaffold sequence insertion as described in FIGS. 60A-60B. FIG. 56A shows an analysis for the EMX1 locus. Shown is the % of total sequencing reads containing one or more pegRNA scaffold sequence nucleotides within an insertion adjacent to the RT template (left); the percentage of total sequencing reads containing a pegRNA scaffold sequence insertion of the specified length (middle); and the cumulative total percentage of pegRNA insertion up to and including the length specified on the X axis. FIG. 56B shows the same as FIG. 56A, but for FANCF. FIG. 56C shows the same as in FIG. 56A but for HEK3. FIG. 56D shows the same as FIG. 56A but for RNF2. Values and error bars reflect the mean and s.d. of three independent biological replicates.

FIGS. 57A-57I show the effects of PE2, PE2-dRT, and Cas9 H840A nickase on transcriptome-wide RNA abundance. Analysis of cellular RNA, depleted for ribosomal RNA, isolated from HEK293T cells expressing PE2, PE2-dRT, or Cas9 H840A nickase and a PRNP-targeting or HEXA-targeting pegRNA. RNAs corresponding to 14,410 genes and 14,368 genes were detected in PRNP and HEXA samples, respectively. FIGS. 57A-57F show Volcano plot displaying the −log 10 FDR-adjusted p-value vs. log 2-fold change in transcript abundance for each RNA, comparing (FIG. 57A) PE2 vs. PE2-dRT with PRNP-targeting pegRNA, (FIG. 57B) PE2 vs. Cas9 H840A with PRNP-targeting pegRNA, (FIG. 57C) PE2-dRT vs. Cas9 H840A with PRNP-targeting pegRNA, (FIG. 57D) PE2 vs. PE2-dRT with HEXA-targeting pegRNA, (FIG. 57E) PE2 vs. Cas9 H840A with HEXA-targeting pegRNA, (FIG. 57F) PE2-dRT vs. Cas9 H840A with HEXA-targeting pegRNA. Red dots indicate genes that show ≥2-fold change in relative abundance that are statistically significant (FDR-adjusted p<0.05). FIGS. 57G-57I are Venn diagrams of upregulated and downregulated transcripts (≥2-fold change) comparing PRNP and HEXA samples for (FIG. 57G) PE2 vs PE2-dRT, (FIG. 57H) PE2 vs. Cas9 H840A, and (FIG. 57I) PE2-dRT vs. Cas9 H840A.

FIG. 58 shows representative FACS gating for neuronal nuclei sorting. Nuclei were sequentially gated on the basis of DyeCycle Ruby signal, FSC/SSC ratio, SSC-Width/SSC-height ratio, and GFP/DyeCycle ratio.

FIGS. 59A-59F show the protocol for cloning 3′-extended pegRNAs into mammalian U6 expression vectors by Golden Gate assembly. FIG. 59A shows the cloning overview. FIG. 59B shows ‘Step 1: Digest pU6-pegRNA-GG-Vector plasmid (component 1)’. FIG. 59C shows ‘Steps 2 and 3: Order and anneal oligonucleotide parts (components 2, 3, and 4)’. FIG. 59D shows ‘Step 2.b.ii.: sgRNA scaffold phosphorylation (unnecessary if oligonucleotides were purchased phosphorylated)’. FIG. 59E shows ‘Step 4: pegRNA assembly’. FIG. 59F shows ‘Steps 5 and 6: Transformation of assembled plasmids’. FIG. 59F shows a diagram summarizing the pegRNA cloning protocol.

FIGS. 60A-60B show the Python script for quantifying pegRNA scaffold integration. A custom python script was generated to characterize and quantify pegRNA insertions at target genomic loci. The script iteratively matches text strings of increasing length taken from a reference sequence (guide RNA scaffold sequence) to the sequencing reads within fastq files, and counts the number of sequencing reads that match the search query. Each successive text string corresponds to an additional nucleotide of the guide RNA scaffold sequence. Exact length integrations and cumulative integrations up to a specified length were calculated in this manner. At the start of the reference sequence, 5 to 6 bases of the 3′ end of the new DNA strand synthesized by the reverse transcriptase are included to ensure alignment and accurate counting of short slices of the sgRNA.

FIG. 61 is a graph showing the percent of total sequencing reads with the specified edit for SaCas9(N580A)-MMLV RT HEK3+6 C>A. The values for the correct edits as well as indels are shown.

FIGS. 62A-62B show the importance of the protospacer for efficient installation of a desired edit at a precise location with prime editing. FIG. 62A is a graph showing the percent of total sequencing reads with target T•A base pairs converted to A•T for various HEK3 loci. FIG. 62B is a sequence analysis showing the same.

FIG. 63 is a graph showing SpCas9 PAM variants in PAM editing (N=3). The percent of total sequencing reads with the targeted PAM edit is shown for SpCas9(H840A)-VRQR-MMLV RT, where NGA>NTA, and for SpCas9(H840A)-VRER-MMLV RT, where NGCG>NTCG. The pegRNA primer binding site (PBS) length, RT template (RT) length, and PE system used are listed.

FIG. 64 is a schematic showing the introduction of various site-specific recombinase (SSR) targets into the genome using PE. (a) provides a general schematic of the insertion of a recombinase target sequence by a prime editor. (b) shows how a single SSR target inserted by PE can be used as a site for genomic integration of a DNA donor template. (c) shows how a tandem insertion of SSR target sites can be used to delete a portion of the genome. (d) shows how a tandem insertion of SSR target sites can be used to invert a portion of the genome. (e) shows how the insertion of two SSR target sites at two distal chromosomal regions can result in chromosomal translocation. (f) shows how the insertion of two different SSR target sites in the genome can be used to exchange a cassette from a DNA donor template.

FIG. 65 shows in 1) the PE-mediated synthesis of a SSR target site in a human cell genome and 2) the use of that SSR target site to integrate a DNA donor template comprising a GFP expression marker. Once successfully integrated, the GFP causes the cell to fluoresce.

FIG. 66 depicts one embodiment of a prime editor being provided as two PE half proteins which regenerate as whole prime editor through the self-splicing action of the split-intein halves located at the end or beginning of each of the prime editor half proteins.

FIG. 67 depicts the mechanism of intein removal from a polypeptide sequence and the reformation of a peptide bond between the N-terminal and the C-terminal extein sequences. (a) depicts the general mechanism of two half proteins each containing half of an intein sequence, which when in contact within a cell result in a fully-functional intein which then undergoes self-spicing and excision. The process of excision results in the formation of a peptide bond between the N-terminal protein half (or the “N extein”) and the C-terminal protein half (or the “C extein”) to form a whole, single polypeptide comprising the N extein and the C extein portions. In various embodiments, the N extein may correspond to the N-terminal half of a split prime editor and the C extein may correspond to the C-terminal half of a split prime editor. (b) shows a chemical mechanism of intein excision and the reformation of a peptide bond that joins the N extein half (the red-colored half) and the C extein half (the blue-colored half). Excision of the split inteins (i.e., the N intein and the C intein in the split intein configuration) may also be referred to as “trans splicing” as it involves the splicing action of two separate components provided in trans.

FIG. 68A demonstrates that delivery of both split intein halves of SpPE (SEQ ID NO: 383) at the linker maintains activity at three test loci when co-transfected into HEK293T cells.

FIG. 68B demonstrates that delivery of both split intein halves of SaPE2 (e.g., SEQ ID NO: 394 and SEQ ID NO: 395) recapitulate activity of full length SaPE2 (SEQ ID NO: 33) when co-transfected into HEK293T cells. Residues indicated in quotes are the sequence of amino acids 741-743 in SaCas

9 (first residues of the C-terminal extein) which are important for the intein trans splicing reaction. ‘SMP’ are the native residues, which we also mutated to the ‘CFN’ consensus splicing sequence. The consensus sequence is shown to yield the highest reconstitution as measured by prime editing percentage.

FIG. 68C provides data showing that various disclosed PE ribonucleoprotein complexes (PE2 at high concentration, PE3 at high concentration and PE3 at low concentration) can be delivered in this manner.

FIG. 69 shows a bacteriophage plaque assay to determine PE effectiveness in PANCE. Plaques (dark circles) indicate phage able to successfully infect E. coli. Increasing concentration of L-rhamnose results in increased expression of PE and an increase in plaque formation. Sequencing of plaques revealed the presence of the PE-installed genomic edit.

FIGS. 70A-70I provide an example of an edited target sequence as an illustration of a step-by-step instruction for designing pegRNAs and nicking-sgRNAs for prime editing. FIG. 70A: Step 1. Define the target sequence and the edit. Retrieve the sequence of the target DNA region (˜200 bp) centered around the location of the desired edit (point mutation, insertion, deletion, or combination thereof). FIG. 70B: Step 2. Locate target PAMs. Identify PAMs in proximity to the edit location. Be sure to look for PAMs on both strands. While PAMs close to the edit position are preferred, it is possible to install edits using protospacers and PAMs that place the nick ≥30 nt from the edit position. FIG. 70C: Step 3. Locate the nick sites. For each PAM being considered, identify the corresponding nick site. For Sp Cas9 H840A nickase, cleavage occurs in the PAM-containing strand between the 3^{rd} and 4^{th} bases 5′ to the NGG PAM. All edited nucleotides must exist 3′ of the nick site, so appropriate PAMs must place the nick 5′ to the target edit on the PAM-containing strand. In the example shown below, there are two possible PAMs. For simplicity, the remaining steps will demonstrate the design of a pegRNA using PAM 1 only. FIG. 70D: Step 4. Design the spacer sequence. The protospacer of Sp Cas9 corresponds to the 20 nucleotides 5′ to the NGG PAM on the PAM-containing strand. Efficient Pol III transcription initiation requires a G to be the first transcribed nucleotide. If the first nucleotide of the protospacer is a G, the spacer sequence for the pegRNA is simply the protospacer sequence. If the first nucleotide of the protospacer is not a G, the spacer sequence of the pegRNA is G followed by the protospacer sequence. FIG. 70E: Step 5. Design a primer binding site (PBS). Using the starting allele sequence, identify the DNA primer on the PAM-containing strand. The 3′ end of the DNA primer is the nucleotide just upstream of the nick site (i.e. the 4″ base 5′ to the NGG PAM for Sp Cas9). As a general design principle for use with PE2 and PE3, a pegRNA primer binding site (PBS) containing 12 to 13 nucleotides of complementarity to the DNA primer can be used for sequences that contain ˜40-60% GC content. For sequences with low GC content, longer (14- to 15-nt) PBSs should be tested. For sequences with higher GC content, shorter (8- to 11-nt) PBSs should be tested. Optimal PBS sequences should be determined empirically, regardless of GC content. To design a length-p PBS sequence, take the reverse complement of the first p nucleotides 5′ of the nick site in the PAM-containing strand using the starting allele sequence. FIG. 70F: Step 6. Design an RT template. The RT template encodes the designed edit and homology to the sequence adjacent to the edit. Optimal RT template lengths vary based on the target site. For short-range edits (positions +1 to +6), it is recommended to test a short (9 to 12 nt), a medium (13 to 16 nt), and a long (17 to 20 nt) RT template. For long-range edits (positions +7 and beyond), it is recommended to use RT templates that extend at least 5 nt (e.g., 10 or more nt) past the position of the edit to allow for sufficient 3′ DNA flap homology. For long-range edits, several RT templates should be screened to identify functional designs. For larger insertions and deletions (≥5 nt), incorporation of greater 3′ homology (˜20 nt or more) into the RT template is recommended. Editing efficiency is typically impaired when the RT template encodes the synthesis of a G as the last nucleotide in the reverse transcribed DNA product (corresponding to a C in the RT template of the pegRNA). As many RT templates support efficient prime editing, avoidance of G as the final synthesized nucleotide is recommended when designing RT templates. To design a length-r RT template sequence, use the desired allele sequence and take the reverse complement of the first r nucleotides 3′ of the nick site in the strand that originally contained the PAM. Note that compared to SNP edits, insertion or deletion edits using RT templates of the same length will not contain identical homology. FIG. 70G: Step 7. Assemble the full pegRNA sequence. Concatenate the pegRNA components in the following order (5′ to 3′): spacer, scaffold, RT template and PBS. FIG. 70H: Step 8. Designing nicking-sgRNAs for PE3. Identify PAMs on the non-edited strand upstream and downstream of the edit. Optimal nicking positions are highly locus-dependent and should be determined empirically. In general, nicks placed 40 to 90 nucleotides 5′ to the position across from the pegRNA-induced nick lead to higher editing yields and fewer indels. A nicking sgRNA has a spacer sequence that matches the 20-nt protospacer in the starting allele, with the addition of a 5′-G if the protospacer does not begin with a G. FIG. 70I: Step 9. Designing PE3b nicking-sgRNAs. If a PAM exists in the complementary strand and its corresponding protospacer overlaps with the sequence targeted for editing, this edit could be a candidate for the PE3b system. In the PE3b system, the spacer sequence of the nicking-sgRNA matches the sequence of the desired edited allele, but not the starting allele. The PE3b system operates efficiently when the edited nucleotide(s) falls within the seed region (˜10 nt adjacent to the PAM) of the nicking-sgRNA protospacer. This prevents nicking of the complementary strand until after installation of the edited strand, preventing competition between the pegRNA and the sgRNA for binding the target DNA. PE3b also avoids the generation of simultaneous nicks on both strands, thus reducing indel formation significantly while maintaining high editing efficiency. PE3b sgRNAs should have a spacer sequence that matches the 20-nt protospacer in the desired allele, with the addition of a 5′ G if needed.

FIG. 71A shows the nucleotide sequence of a SpCas9 pegRNA molecule (top) which terminates at the 3′ end in a “UUU” and does not contain a toeloop element. The lower portion of the figure depicts the same SpCas9 pegRNA molecule but is further modified to contain a toeloop element having the sequence 5′-“GAAANNNNN”-3′ inserted immediately before the “UUU” 3′ end. The “N” can be any nucleobase.

FIG. 71B shows the results of Example 3, which demonstrates that the efficiency of prime editing in HEK cells or EMX cells is increased using pegRNA containing toeloop elements, whereas the percent of indel formation is largely unchanged.

FIG. 72 depicts alternative pegRNA configurations that can be used in prime editing. (a) Depicts the PE2:pegRNA embodiment of prime editing. This embodiment involves a PE2 (a fusion protein comprising a Cas9 and a reverse transcriptase) complexed with a pegRNA (as also described in FIGS. 1A-1I and/or FIG. 3A-3E). In this embodiment, the template for reverse transcription is incorporated into a 3′ extension arm on the sgRNA to make the pegRNA, and the DNA polymerase enzyme is a reverse transcriptase (RT) fused directly to Cas9. (b) Depicts the MS2cp-PE2:sgRNA+tPERT embodiment. This embodiment comprises a PE2 fusion (Cas9+a reverse transcriptase) that is further fused to the MS2 bacteriophage coat protein (MS2cp) to form the MS2cp-PE2 fusion protein. To achieve prime editing, the MS2cp-PE2 fusion protein is complexed with an sgRNA that targets the complex to a specific target site in the DNA. The embodiment then involves the introduction of a trans prime editing RNA template (“tPERT”), which operates in place of a pegRNA by providing a primer binding site (PBS) and an DNA synthesis template on separate molecule, i.e., the tPERT, which is also equipped with a MS2 aptamer (stem loop). The MS2cp protein recruits the tPERT by binding to the MS2 aptamer of the molecule. (c) Depicts alternative designs for pegRNAs that can be achieved through known methods for chemical synthesis of nucleic acid molecules. For example, chemical synthesis can be used to synthesize a hybrid RNA/DNA pegRNA molecule for use in prime editing, wherein the extension arm of the hybrid pegRNA is DNA instead of RNA. In such an embodiment, a DNA-dependent DNA polymerase can be used in place of a reverse transcriptase to synthesize the 3′ DNA flap comprising the desired genetic change that is formed by prime editing. In another embodiment, the extension arm can be synthesized to include a chemical linker that prevents the DNA polymerase (e.g., a reverse transcriptase) from using the sgRNA scaffold or backbone as a template. In still another embodiment, the extension arm may comprise a DNA synthesis template that has the reverse orientation relative to the overall orientation of the pegRNA molecule. For example, and as shown for a pegRNA in the 5′-to-3′ orientation and with an extension attached to the 3′ end of the sgRNA scaffold, the DNA synthesis template is orientated in the opposite direction, i.e., the 3′-to-5′ direction. This embodiment may be advantageous for pegRNA embodiments with extension arms positioned at the 3′ end of a gRNA. By reverse the orientation of the extension arm, the DNA synthesis by the polymerase (e.g., reverse transcriptase) will terminate once it reaches the newly orientated 5′ of the extension arm and will thus, not risk using the gRNA core as a template.

FIG. 73 demonstrates prime editing with tPERTs and the MS2 recruitment system (aka MS2 tagging technique). An sgRNA targeting the prime editor protein (PE2) to the target locus is expressed in combination with a tPERT containing a primer binding site (a13-nt or 17-nt PBS), an RT template encoding a His6 tag insertion and a homology arm, and an MS2 aptamer (located at the 5′ or 3′ end of the tPERT molecule). Either prime editor protein (PE2) or a fusion of the MS2cp to the N-terminus of PE2 was used. Editing was carried out with or without a complementary-strand nicking sgRNA, as in the previously developed PE3 system (designated in the x-axis as labels “PE2+nick” or “PE2”, respectively). This is also referred to and defined herein as “second-strand nicking.”

FIG. 74 demonstrates that the MS2 aptamer expression of the reverse transcriptase in trans and its recruitment with the MS2 aptamer system. The pegRNA contains the MS2 RNA aptamer inserted into either one of two sgRNA scaffold hairpins. The wild-type M-MLV reverse transcriptase is expressed as an N-terminal or C-terminal fusion to the MS2 coat protein (MCP). Editing is at the HEK3 site in HEK293T cells.

FIG. 75 provides a bar graph comparing the efficiency (i.e., “% of total sequencing reads with the specified edit or indels”) of PE2, PE2-trunc, PE3, and PE3-trunc over different target sites in various cell lines. The data shows that the prime editors comprising the truncated RT variants were about as efficient as the prime editors comprising the non-truncated RT proteins.

FIG. 76 demonstrates the editing efficiency of intein-split prime editors. HEK239T cells were transfected with plasmids encoding full-length PE2 or intein-split PE2, pegRNA and nicking guide RNA. Consensus sequence (most amino-terminal residues of C terminal extein) are indicated. Percent editing at two sites in shown: HEK3+1 CTT insertion and PRNP +6 G to T. Replicate n=3 independent transfections.

FIG. 77 demonstrates the editing efficiency of intein-split prime editors. Editing assessed by targeted deep sequencing in bulk cortex and GFP+ subpopulation upon delivery of 5E10 vg per SpPE3 half and a small amount 1E10 of nuclear-localized GFP:KASH to P0 mice by ICV injection. Editors and GFP were packaged in AAV9 with EFS promoter. Mice were harvested three weeks post injection and GFP+ nuclei were isolated by flow cytometry. Individual data points are shown, with 1-2 mice per condition analyzed.

FIG. 78 demonstrates the editing efficiency of intein-split prime editors. Specifically, the figures depicts the AAV split-SpPE3 constructs. Co-transduction by AAV particles separately expressing SpPE3-N and SpPE3-C recapitulates PE3 activity. Note N-terminal genome contains a U6-sgRNA cassette expressing the nicking sgRNA, and the C-terminal genome contains a U6-pegRNA cassette expressing the pegRNA.

FIG. 79 shows the editing efficiency of certain optimized linkers. In particular, the data shows the editing efficiency of the PE2 construct with the current linker (noted as PE2—white box) compared to various versions with the linker replaced with a sequence as indicated at the HEK3, EMX1, FANCF, RNF2 loci for representative pegRNAs for transition, transversion, insertion, and deletion edits. The replacement linkers are referred to as “1×SGGS” (SEQ ID NO: 8), “2×SGGS” (SEQ ID NO: 9), “3×SGGS” (SEQ ID NO: 10), “1×XTEN” (SEQ ID NO: 11), “no linker”, “1×Gly”, “1×Pro”, “1×EAAAK” (SEQ ID NO: 12), “2×EAAAK” (SEQ ID NO: 13), and “3×EAAAK” (SEQ ID NO: 14). The editing efficiency is measured in bar graph format relative to the “control” editing efficiency of PE2. The linker of PE2 is SGGSSGGSSGSETPGTSESATPESSGGSSGGSS (SEQ ID NO: 11). All editing was done in the context of the PE3 system, i.e., which refers the PE2 editing construct plus the addition of the optimal secondary sgRNA nicking guide.

FIG. 80. Taking the average fold efficacy relative to PE2 yields the graph shown, indicating that use of a 1×XTEN linker sequence improves editing efficiency by 1.14 fold on average (n=15).

FIG. 81 depicts the transcription level of pegRNAs from different promoters, as described in Example 2.

FIG. 82 As depicted in Example 2, impact of different types of modifications on pegRNA structure on editing efficiency relative to unmodified pegRNA.

FIG. 83 Depicts a PE experiment that targeted editing of the HEK3 gene, specifically targeting the insertion of a 10 nt insertion at position +1 relative to the nick site and using PE3. See Example 2.

FIG. 84A depicts structure of tRNA that can be used to modify pegRNA structures. See Example 2. The P1 can be variable in length. The P1 can be extended to help prevent RNAseP processing of the pegRNA-tRNA fusion.

FIG. 84B depicts an exemplary pegRNA having a spacer, gRNA core, and an extension arm (RT template+primer binding site), which is modified at the 3′ end of the pegRNA with a tRNA molecule, coupled through a UCU linker. The tRNA includes various post-transcriptional modifications. Said modification are not required, however. See Example 2.

FIG. 85 depicts a PE experiment that targeted editing of the FANCF gene, specifically targeting a G-to-T conversion at position +5 relative to the nick site and using PE3 construct. See Example 2.

FIG. 86 depicts a PE experiment that targeted editing of the HEK3 gene, specifically targeting the insertion of a 71 nt FLAG tag insertion at position +1 relative to the nick site and using PE3 construct. See Example 2.

FIG. 87 results from a screen in N2A cells where the pegRNA installs 1412Adel, with details about the primer binding site (PBS) length and reverse transcriptase (RT) template length. (Shown with and without indels).

FIG. 88 results from a screen in N2A cells where the pegRNA installs 1412Adel, with details about the primer binding site (PBS) length and reverse transcriptase (RT) template length. (Shown with and without indels).

FIG. 89 depicts results of editing at a proxy locus in the β-globin gene and at HEK3 in healthy HSCs, varying the concentration of editor to pegRNA and nicking gRNA.

FIG. 90A shows RT-qPCR data demonstrating that using in vitro transcribed pegRNA, which is undegraded and full length, PCR amplicons 3 and 6 amplify with the same efficiency as an amplicon consisting of the spacer and scaffold regions of the pegRNA. Amplicon 3 contains the template region of the pegRNA, whereas amplicon 6 contains the PBS of the pegRNA. Bars are the average of 3 technical replicates.

FIG. 90B shows RT-qPCR data demonstrating that the pegRNA template and PBS are reduced in abundance after extraction from cells, particularly the PBS, in comparison to in vitro transcribed pegRNA put through the same extraction process.

FIG. 90C provides the template amplicon and PBS amplicon sequences correspond to amplicon 3 and 6 respectively in the FIG. 90B.

FIG. 91A-91D provides the results of scaffold modifications on pegRNA activity for edits +1FLAG at HEK3 (FIG. 91A), +5G-T at RNF2 (FIG. 91B), +5G-T at DNMT1 (FIG. 91C), and +5G-T at EMX1 (FIG. 91D). Modifications to P1, P2, and P3 of the scaffold broadly kill activity. Modifications to the direct repeat can improve activity.

FIG. 92A-92C provides +1FLAG insertion edits at HEK3 (FIG. 91A), RNF2 (FIG. 91B) and RUNX1 (FIG. 91C) loci. pegRNAs include structural motif and linkers as noted. If the linker length is not given, length is 8.

FIG. 93A-93H shows the results of PE of the indicated edit (in the title as a deletion “del” or point mutation) at the indicated site for pegRNAs with 3′ structural motifs (linker=8; motif=linker+evopreQ₁-1 or linker+MMLV-pknot), linker alone (linker=8; motif no 3′ addition (linker=0; motif=“−”) at a variety of template lengths (15 nucleotides (“template 15”), 25 nucleotides (“template 25”), and 35 nucleotides (“template 35”)). Blue bars indicate % of total sequencing reads with the correct edits. The grey bars track the appearance of indels as a % of total sequencing reads.

FIG. 94 shows the summary of effect of either linker alone, linker+evopreQ₁-1 or linker+Mpknot-1 on prime editing activity, summarizing data in FIGS. 93A-H. Line indicates median fold increase.

FIG. 95A-95B shows the editing efficiency in Hela, U20S, and K562 cells lines for insertion of the nucleotide sequence corresponding to the FLAG tag at the HEK3 locus after plasmid nucleofection. Results are an average of three biological replicates. Results indicate that the increase in efficacy of the 3′ stabilizing modifications may be greater in other cell types where delivery of editing agents is less efficient.

FIG. 96 shows the effect of mutations mut1 and mut2 on prime editing activity. Mutations are predicted to disrupt the structure of evopreQ₁-1.

FIG. 97 shows RT-qPCR data demonstrating that the 3′ structural motifs preserve the 3′ end of the pegRNA, particularly the PBS which is critical for prime editing, versus the unmodified species. Bars are the average of three biological replicates, each of which are the average of three technical replicates. Template amplicon and PBS amplicon correspond to amplicons 3 and 6, respectively.

FIG. 98 provides a schematic of a pegRNA appended at 3′ end with a nucleic acid moiety, which may include, but is not limited to a double helix moiety, a toeloop moiety, a hairpin moiety, a stem-loop moiety, a pseudoknot moiety, an aptamer moiety, a G quadraplex moiety, or a tRNA moiety. The nucleic acid moiety can be joined to the 3′ end of the pegRNA by an optional nucleotide linker (e.g., 3-18 nucleotides).

FIG. 99 is a schematic of an expression vector comprising a U6 promoter, which was surprisingly found to result in improved editing efficiency.

FIG. 100A-100E. Demonstrates that use of U6 promoters (including U6 wildtype, US v4, U6 v7, and U6 v9) to express pegRNAs leads to improved editing.

FIG. 101 shows the folding for evopreq1 nucleic acid moiety which can be used to modify pegRNA.

FIG. 102 shows the folding for Mpknot1 nucleic acid moiety which can be used to modify pegRNA.

FIG. 103 shows the folding for tRNA nucleic acid moiety which can be used to modify pegRNA.

FIGS. 104A-104C show that truncated pegRNAs limit prime editing efficiency. FIG. 104A (left) provides a schematic of a prime editing complex composed of a prime editor (PE) protein that consists of a Cas9 nickase (nCas9) fused to a modified reverse transcriptase via a flexible linker and a prime editing guide RNA (pegRNA). FIG. 104A (right) shows that degradation of the 3′ extension of a pegRNA by exonucleases could impede editing efficiency through loss of the PBS. FIG. 104B shows PE3-mediated editing efficiencies with the addition of plasmids expressing sgRNAs, truncated pegRNAs that target the same genomic locus (HEK3), non-targeting pegRNA, or SaCas9 pegRNAs. All pegRNAs are expressed from a U6 promoter. Data and error bars reflect the mean and standard deviation of three independent biological replicates. FIG. 104C shows the design of engineered pegRNAs (epegRNAs) that contain a structured RNA pseudoknot, which protects the 3′ extension from degradation by exonucleases.

FIGS. 105A-105D show that PE editing efficiency is enhanced by the addition of structured RNA motifs to the 3′ terminus of pegRNAs. FIG. 105A shows the efficiency of PE3-mediated insertions of the FLAG epitope tag at the +1 editing position (insertion directly at the pegRNA-induced nick site) across multiple genomic loci in HEK293T cells using canonical pegRNAs (“unmodified”), pegRNAs with either evopreQ₁ or mpknot motif appended to the 3′ end of the PBS connected via an 8-nt linker sequence, or pegRNAs appended with only the 8-nt linker sequence on the 3′ end. FIG. 105B provides a summary of the fold-change in PE editing efficiency relative to canonical pegRNAs of the indicated edit at various genomic loci upon addition of the indicated 3′ motif via an 8-nt linker, or the addition of the linker alone. “Transversion” denotes mutation of the +5 G•C to T•A at RUNX1, EMX1, VEGFA, and DNMT1, the +1 C•G to T•A at RNF2, and the +1 T•A to A•T at HEK3, where the positive integer indicates the distance from the Cas9 nick site. “Deletion” denotes a 15-bp deletion at the Cas9 nick site. Data summarized here are presented in FIG. 105C and FIGS. 109A-109K. The horizontal bars show the median values. FIG. 105C shows representative improvements in PE editing efficiency as a result of appending either evopreQ₁ (p) or mpknot (m) via an 8-nt linker to pegRNAs with varying template lengths (in nucleotides, indicated). FIG. 105D shows editing activities of canonical pegRNAs and modified pegRNAs across three genomic loci in HeLa cells, U2OS cells, and K562 cells. Data and error bars indicate the mean and standard deviation of three independent biological replicates (FIGS. 105A, 105C, and 105D).

FIGS. 106A-106D show that structural motifs increase the RNA stability and efficiency of reverse transcription. FIG. 106A shows resistance of unmodified pegRNA or epegRNA containing evopreQ₁ or mpknot to degradation upon exposure to HEK293T nuclear lysates. The agarose gel shown is representative of three experiments. Untreated in vitro transcribed pegRNAs or epegRNAs served as standards. Percent RNA remaining was calculated using densitometry. Significance was analyzed using a two-tailed unpaired Student's t test (p=0.0028 for mpknot and 0.0022 for evopreQ1). FIG. 106B shows fold change in abundance of the pegRNA scaffold relative to unmodified pegRNA upon exposure to HEK293T nuclear lysates in the absence and presence of nCas9 as determined by RT-qPCR of the sgRNA scaffold. FIG. 106C shows a comparison of prime-editing intermediates generated by PE2 with either pegRNAs or epegRNAs at RNF2. Dotted lines indicate the full-length reverse transcriptase product templated by the pegRNA or epegRNA tested at the indicated locus. X axis is relative to the position of the PE2-induced nick with the first base 3′ downstream represented as position +1. Histograms and pie charts are generated from the average of three independent biological replicates. FIG. 106D shows PE3 editing efficiencies in HEK293T cells using unmodified pegRNAs, pegRNAs containing the evopreQ1 motif, or pegRNAs containing a G15C point mutant of evopreQ₁ (M1) that disrupts the pseudoknot motif structure. FIG. 106E shows the fraction of Cas9 RNPs composed of dCas9 and either unmodified pegRNA or epegRNA containing either evopreQ1 or mpknot and templating a +1 FLAG tag insertion at HEK3 bound to dsDNA as determined by MST. FIG. 106F shows CRISPRa transcriptional activation by pegRNAs, epegRNAs, and sgRNAs. Reported GFP fluorescence is normalized to iRFP fluorescence expressed from a co-transfected plasmid. AU, arbitrary units. FIG. 106G shows the fraction of unmodified pegRNA or epegRNA (templating a +1 FLAG tag insertion at HEK3) containing either evopreQ1 or mpknot bound to H840A nCas9 as determined by microscale thermophoresis (MST). Data and error bars reflect the mean and standard deviation of three independent biological replicates. FIG. 106H shows the abundance of epegRNA and canonical pegRNA used in FIG. 106A in HEK293T cells by RT-qPCR amplification and quantification of the sgRNA scaffold. Primers can be found in Table E5. FIGS. 107A-107E show that prime editing-mediated editing efficiency of therapeutically relevant genome editing is improved by the use of epegRNAs. FIG. 107A shows PE3-mediated installation of the G127V mutation in PRNP that protects against human prion disease. FIGS. 107B-107C show correction of the pathogenic c1278TATC insertion in HEXA that causes Tay Sachs disease in both HEK293T cells (FIG. 107B) and primary patient-derived fibroblasts (FIG. 107C). FIG. 107D shows a comparison of PE2-mediated installation of pathogenic and protective alleles using unoptimized epegRNAs or unoptimized pegRNAs at nine genomic sites. Reference SNP (rs) designations can be found for all mutations in Table E6. FIG. 107E shows PE2-mediated editing efficiency of FLAG epitope tag insertion at 15 genomic loci in HEK293T cells using unoptimized epegRNAs compared to unoptimized canonical pegRNAs. Data and error bars indicate the mean and standard deviation of three independent biological replicates.

FIG. 108 shows the sequences and secondary structures of RNA structural motifs examined in this study. Structures are based on predictions from previously published structural or bioinformatic analyses. Only two G-quadruplexes of the 11 tested are shown for brevity. Sequences of all motifs are provided in Table E2.

FIGS. 109A-109C show PE3-mediated edit:indel ratio for pegRNAs and epegRNAs shown in FIGS. 105A-105D. Fold-change in the observed prime editing edit:indel ratio for installation of a FLAG epitope tag (FIG. 109A) or the indicated transversion or deletion (FIG. 109B) in HEK293T cells, or the indicated edit in HeLa, U20S, or K562 cells (FIG. 109C) of epegRNAs bearing either evopreQ1 (p) or mpknot (m) compared to unmodified pegRNA (dashed line). Values were calculated from the data presented in FIGS. 105A, 105C, and 105D, respectively. Data and error bars reflect the mean and standard deviation of three independent biological replicates.

FIG. 110 shows the linker-length dependence of epegRNA activity. Effect of removing the 8-nt linkers used in FIGS. 105A-105D and FIGS. 111A-111K on PE3 editing efficiency. Either evopreQ1 (p) or mpknot (m) was appended to the PBS via either no linker or an 8-nt linker. The distance from the Cas9 nick site to the installed mutation in nucleotides is as indicated in the legend. Dots indicate the average of three biological replicates. Bars indicate the grand median. Significance was calculated via a two-tailed paired Student's t test (p=0.022).

FIGS. 111A-111K show improvement in PE3-mediated editing efficiency at various genomic loci from to the addition of 3′ RNA structural motifs to pegRNAs. FIGS. 111A-111K show PE3-mediated installation of the indicated edit at DNMT1 (FIGS. 111A-111B), RUNX1 (FIG. 111C), RNF2 (FIGS. 111D-109E), FANCF (FIGS. 111F-111G), EMX1 (FIGS. 111H-111I), VEGFA (FIG. 111J), and HEK3 (FIG. 111K). Either an 8-nt linker alone or the linker in conjunction with evopreQ1 (p) or mpknot (m) was appended to pegRNAs of increasing template lengths and compared to canonical pegRNAs. The distance from the Cas9 nick site to the installed mutation is indicated. Error bars indicate the standard deviation of three replicates.

FIGS. 112A-112C show PE3-mediated edit:indel ratio for pegRNAs and epegRNAs shown in FIG. 110. Fold-change in the observed edit:indel ratio for the indicated transversion or deletion at HEK3, RUNX1, or DNMT1 (FIG. 112A), RNF2 or FANCF (FIG. 112B), or EMX1 or VEGFA (FIG. 112C) of epegRNAs bearing either evopreQ1 (p) or mpknot (m) compared to unmodified pegRNA (dashed line). Values were calculated from the data presented in FIGS. 109A-109C. Data and error bars reflect the mean and standard deviation of three independent biological replicates.

FIG. 113 shows that the engineered pegRNAs demonstrate no increase in detected off-target activity compared to canonical pegRNAs. On- and off-target PE3 editing of pegRNAs and epegRNAs targeted to HEK3, EMX1, or FANCF and templating either a nucleotide transversion (T•A to A•T at HEK3 or G•C to T•A at EMX1 and FANCF; pt mtn) or a 15-nt deletion (del); −, canonical pegRNA; m, epegRNA containing mpknot; p, epegRNA containing evopreQ1. Indel frequencies are shown in parentheses. For EMX1 off-target 1, indels were obtained by subtracting the percentage of sequencing reads containing indels in cells transfected with a non-targeting pegRNA. Off-target loci are listed in Table E4. Data are the average of three biological replicates. FIGS. 114A-114C shows site-dependent expression differences of pegRNAs and epegRNAs. Northern blot of HEK293T lysates containing pegRNAs or epegRNAs targeted to (FIG. 114A) HEK3 or (FIG. 114B) EMX1 after hybridization with a DIG-labeled RNA probe complementary to the sgRNA scaffold. PAGE gels shown are representative of multiple independent biological replicates. The normalized fold change in abundance relative to unmodified pegRNA as determined by densitometry is shown (right). Abundance was calculated by including both full-length pegRNA and epegRNA for samples in which full length pegRNA is present. Band identity was confirmed using untreated in vitro transcribed pegRNAs and epegRNAs as standards, DIG-labeled ssRNA ladder, and purified RNA from HEK293T cells transfected with sgRNA as markers. FIG. 114C shows the abundance of epegRNA and canonical pegRNA targeted to HEK3, DNMT1, RNF2 or EMX1 in HEK293T cells by RT-qPCR amplification and quantification of the sgRNA scaffold. Primers for qPCR amplification can be found in Table E5. Data and error bars reflect the mean and standard deviation of three independent biological replicates.

FIGS. 115A-115C show high-throughput sequencing analysis of PE2-mediated genomic reverse transcriptase products. Comparison of prime-editing intermediates generated by PE2 with either pegRNAs or epegRNAs at (FIG. 115A) HEK3, (FIG. 115B) DNMT1, or (FIG. 115C) EMX1 as indicated. Dotted lines indicate the full-length reverse transcriptase product templated by the pegRNA or epegRNA tested at the indicated locus. X axis is relative to the position of the PE2-induced nick with the first base 3′ downstream represented as position +1. Histograms and pie charts are generated from the average of three independent biological replicates.

FIGS. 116A-116D show PE3-mediated editing efficiency of pegRNAs containing other RNA structural motifs. FIGS. 116A-116B show a comparison of PE3-mediated editing efficiencies for the installation of the FLAG epitope tag, a 15-nt deletion, or a point mutation at HEK3 (FIG. 116A) and RNF2 (FIG. 116B) with epegRNAs to which various G-quadruplexes have been appended via an 8-nt linker. G-quadruplexes are ordered based on melting temperature, ranging from 60 to >90° C., as previously determined. FIG. 116C shows PE3-mediated efficiency of installation of point mutations at the indicated genomic loci using pegRNAs containing the evopreQ1 motif or a 15-bp (34-nt) hairpin. FIG. 116D shows that the addition of either a pseudoknot known to inhibit the 5′ exonuclease XrnI (xrn1) or a large tertiary RNA structure (the P4-P6 domain of the group I intron from Tetrahymena thermophila) to the 3′ terminus of the pegRNA via an 8-nt linker does not yield more efficient editing than addition of either evopreQ1 or mpnkot by the same linker. The distance from the Cas9 nick site to the installed mutation is indicated. Data and error bars indicate the standard deviation of three independent biological replicates.

FIGS. 117A-117C show PE3-mediated editing efficiency of epegRNAs containing evopreQ₁ or mpknot variants. A comparison of PE3-mediated editing efficiencies is shown for the installation of the FLAG epitope tag, a 15-nt deletion, or a point mutation at HEK3 and RNF2 with epegRNAs containing various RNA motifs, where the distance between the Cas9 nick and the edit is indicated by +1. FIGS. 117A-117B show PE3 editing efficiencies of additional evolved prequeosine₁-1 riboswitch aptamer variants (FIG. 117A) or modifications to mpknot (FIG. 117B) compared to evopreQ₁ or mpknot. FIG. 117C shows PE3 editing efficiencies of epegRNAs trimmed to remove nucleotides 5′ and 3′ of evopreQ₁ (tevopreQ₁) and mpknot (tmpknot) compared to parent epegRNAs. Data and error bars indicate the mean and standard deviation of three independent biological replicates.

FIG. 118 shows the effect of the (F+E) scaffold on PE2-editing efficiency with lentivirally transduced epegRNAs. PE2-editing efficiency of lentivirally-transduced prime editor and pegRNA or epegRNA that contain tevopreQ1 and either the canonical or (F+E) sgRNA scaffold and that template the indicated edit at HEK3 or DNMT1 in HEK293T cells. Data and error bars reflect the mean and standard deviation of three independent biological replicates.

FIG. 119 shows the effect of (F+E) scaffold modifications on prime editing efficiency with epegRNAs. Comparison of PE3-mediated editing efficiencies of epegRNAs with the indicated scaffold to epegRNAs with the standard SpCas9 sgRNA scaffold. One-tenth the normal amount of plasmids encoding PE2 and pegRNA or epegRNA was transfected in HEK293T cells in these experiments. Edits templated were either a transversion at PRNP, RUNX1, or EMX1 or a 15-nt deletion at HEK3. Modified scaffold sequences all contain the “flip and extension” (F+E) modification. Scaffolds designated cr also contain mutations to the (F+E) scaffold previously identified as potentially improving Cas9 nuclease activity at some sites⁶. Sequences of all scaffolds can be found in Table E1. Lines indicate the grand medians.

FIGS. 120A-120F show computational prediction of effective linker sequences between the PBS and structural motif of epegRNAs. FIG. 120A provides a schematic illustrating the workflow of pegLIT, a computational script to select appropriate linker sequences for epegRNAs. Potential linker sequences are filtered by sequence identity and propensity for base pairing to other regions of the epegRNA. Sequences passing the filter are then optionally clustered based on identity and individual sequences are selected from different clusters to promote diversity in the final output. FIGS. 120B-120C show that epegRNAs containing evopreQ₁ connected via linker sequences recommended by pegLIT lead to modestly improved PE editing efficiency compared to epegRNAs containing evopreQ₁ connected via a human-designed linker or linkers that were predicted by pegLIT to interact with the PBS. FIG. 120D shows rescued activity at those sites at which epegRNAs did not initially yield improvements (FIGS. 111A-111K). FIG. 120E shows that a comparison of PE3-mediated editing efficiencies of epegRNAs with evopreQ₁ and either 8- or 18-nt long linkers suggests no significant improvement is achieved by increasing linker length. FIG. 120F shows a comparison of PE3-mediated editing efficiencies of epegRNAs with either evopreQ₁ (p) or mpknot (m) and either an 8-nt pegLIT linker (8) or no linker (0). Significance was calculated using student's t test (p=0.0061). FIG. 120G shows the fold increase in PE3-mediated editing efficiencies of epegRNAs with tevopreQ1 containing an 8-nt pegLIT linker compared to no linker. Data are presented as the mean with error bars indicating either (FIG. 120B) the standard deviation of the mean for five pegLIT-designed linkers, each in triplicate, or the standard deviation of three replicates for manually designed linker sequences, (FIGS. 120C, 120D, and 120G) the standard deviation of three replicates, or (FIGS. 120E-120F) or the grand mean of the average fold-change in editing efficiency for each indicated site and edit.

FIGS. 121A-121B show improvements in editing efficiency upon nucleofection of chemically synthesized epegRNAs. FIG. 121A shows efficiency of PE3-mediated installation of the indicated edit upon nucleofection of mRNA which encodes PE2, a chemically synthesized nicking sgRNA, and either chemically synthesized pegRNA or epegRNA containing evopreQ₁ via an 8-nt linker is shown. FIG. 121B shows Observed fold-change in the edit:indel ratio for epegRNAs compared to pegRNAs for the indicated site and edit, based on data in FIG. 121A. Data and error bars indicate the standard deviation of two or more independent biological replicates.

FIGS. 122A-122B show PE2-mediated efficiency of installation of FLAG tags at the indicated genomic sites. FIG. 122A shows PE2-mediated editing efficiency of FLAG epitope tag insertion at 15 genomic loci in HEK293T cells using unoptimized epegRNAs compared to unoptimized canonical pegRNAs. FIG. 122B shows data from FIG. 122A shown in bar chart form. Sites with sub 1% editing efficiency with both pegRNAs and epegRNAs are not shown but are listed in Table E1. Data and error bars reflect the mean and standard deviation of three independent biological replicates.

FIG. 123 provides an image of the uncropped agarose gel from FIG. 106A. Uncropped image of the agarose gel used for FIG. 106A with the excerpted region outlined in black. Untreated in vitro transcribed pegRNAs or epegRNAs were used as molecular weight standards.

FIGS. 124A-124C show uncropped northern blots in FIGS. 114A-114C. FIG. 124A shows an uncropped image of the northern blot used for FIG. 114A with the excerpted region outlined in black. Species lengths were confirmed using untreated in vitro transcribed pegRNA and epegRNA as molecular weight standards on a separate blot with a molecular weight ladder (shown in FIG. 124B). FIG. 124B shows an uncropped image of the northern blot used to confirm the band identities and molecular weights of standards in FIG. 124A. FIG. 124C shows an uncropped image of the northern blot used for FIG. 114B with the excerpted region outlined in black.

FIGS. 125A-125E show the effect of various sgRNA scaffolds on editing efficiency in HEK293T cells.

FIGS. 126A-126B show that flip and extension modifications can improve prime editing efficiency in certain instances.

FIGS. 127A-127B show that various sgRNA scaffolds can improve prime editing efficiency in certain instances.

FIG. 128 is a flowchart of an illustrative process 11800 for identifying one or more nucleic acid linkers for coupling a prime editing guide RNA to a nucleic acid moiety, in accordance with some embodiments of the technology described herein. The process 11800 may be implemented using any suitable computing device(s), as aspects of the technology described herein are not limited in this respect.

FIG. 129 is a flowchart of an illustrative process 11900 for iteratively identifying one or more nucleic acid linkers for coupling a prime editing guide RNA to a nucleic acid moiety, in accordance with some embodiments of the technology described herein. The process 11900 may be implemented using any suitable computing device(s), as aspects of the technology described herein are not limited in this respect.

FIG. 130 shows an illustrative implementation of a computer system 12000 in which embodiments of the technology described herein may be implemented. For example, any of the computing devices described herein may be implemented as computing system 12000. The computing system 12000 may include one or more computer hardware processors 12002 and one or more articles of manufacture that comprise non-transitory computer-readable storage media (e.g., memory 12004 and one or more non-volatile storage devices 12006). The processor 12002(s) may control writing data to and reading data from the memory 12004 and the non-volatile storage device(s) 12006 in any suitable manner. To perform any of the functionality described herein, the processor(s) 12002 may execute one or more processor-executable instructions stored in one or more non-transitory computer-readable storage media (e.g., the memory 12004), which may serve as non-transitory computer-readable storage media storing processor-executable instructions for execution by the processor(s) 12002.

FIG. 131 shows three broad areas in which prime editing can be improved. These include recognition of the target nucleic acid, installation of the edit(s), and resolution of the edited DNA heteroduplex.

FIG. 132 shows that PBS:spacer interactions limit PE efficiency by reducing Cas9 affinity but are necessary in order for PBS:protospacer binding to occur. A shorter PBS is shown to result in improved binding affinity to Cas9.

FIG. 133 shows that toeholds can inhibit both PBS:spacer and PBS:protospacer interactions if independent of Cas9 binding.

FIG. 134 shows that toeholds are competed off by PE2 binding due to competitive RNA-protein interactions. Design considerations include 1) the interdependence of the lengths of both Cas9-RT and RT-MS2 linker, the pegRNA extension and PBS, toehold, and linker between MS2 aptamer and toehold; 2) toehold length dependence upon PBS melting temperature and site accessibility; 3) optimization for each site; and 4) tolerance for a non-interacting 17 nucleotide PBS.

FIG. 135 shows that C-terminal MS2 fusions display superior editing efficiency to N-terminal fusions at HEK3.

FIG. 136 shows that MS2 tagging of PE2 provides benefits compared to untagged PE2. PE2-MS2 fusions comprising an xten-16aa linker or an xten-33aa linker are shown.

FIG. 137 shows that MS2 and toeloop tagging rescues long primer binding sites. PE2-MS2 fusions comprising an xten-16aa linker or an xten-33aa linker are shown.

FIG. 138 shows moving the pegRNA extension onto the nicking guide to completely avoid PBS-spacer interactions. Design considerations include: 1) the impact of an extended template as a linker on flap resolution; 2) optimization of nicking spacer; and 3) the need for both PE complexes to be present on the genome simultaneously.

FIG. 139 shows that the strategy shown in FIG. 138 (moving the pegRNA extension onto the nicking guide) enables prime editing.

FIG. 140 shows a model based on mismatch identity and position within the PBS relative to the nick.

FIG. 141 shows that mutations to the PBS are tolerated or in some circumstances enhance PE activity and fit an initial model where mutation location and identity determine PE efficiency.

FIG. 142 shows that longer PBS (RNF2, 15 nt) do not tolerate mutations, potentially because they inhibit PBS:protospacer interactions excessively.

FIG. 143 shows that mutations to the PBS can improve PE efficiency for pegRNAs with shorter optimal PBS's. MutPBS epegRNAs have a mutPBS of 17 with 4 consecutive mutations (HEK3, DNMT1, PRNP) or a mutPBS of fifteen with four consecutive mutations (RNF2), followed by an 8 nt linker and tevopreQ₁.

FIG. 144 shows that mutPBS improvements can provide additional enhancements in editing efficiency when used in combination with epegRNAs.

FIG. 145 demonstrates that prime editing (e.g., with PE3) can be used to install or correct pathogenic alleles and sequence tags.

FIG. 146 demonstrates an embodiment of a prime editing strategy to install and correct CDKL5 c.1412delA mutation.

FIG. 147 demonstrates that prime editing using the pegRNA of FIG. 146 can be used to edit the CDKL5 c.1412delA mutation in human cells.

FIG. 148 demonstrates that a single prime editor (e.g., PE2) complexed with a single pegRNA is capable of correcting a multitude of pathogenic variants in the CDKL5 gene in exon 8, including correcting the V172I, A173D, R175S, W176G, W176R, Y177C, R178P, P180L, E181A, and L182P mutations.

FIG. 149 demonstrates that a single prime editor (e.g., PE2) complexed with a single pegRNA is capable of correcting a multitude of pathogenic mutations at positions +4, +8, +12, +17, +21, and +25 relative to position 1 of the PAM sequence (i.e., the nucleotide in the 5′-most position).

FIG. 150 shows CDKL5 c1412delA prime editing transfection in N2A cells.

FIG. 151 shows editing efficiency of a 1412delA insertion in N2A cells using epegRNA 072 with no seed editing.

FIG. 152 shows editing efficiency of a 1412delA insertion in N2A cells using PE5 and various pegRNAs with the addition of a seed edit.

FIG. 153 shows editing efficiency of installation of multiple pathogenic CDKL5 alleles in HEK293T cells via plasmid transfection.

FIG. 154 shows a schematic of PE2 and PEmax editor architectures. bpNLSSV40, bipartite SV40 NLS nuclear localization signal. MMLV RT, Moloney Murine Leukemia Virus reverse transcriptase pentamutant; codon opt., human codon-optimized.

FIG. 155 compares the structure of PE2, PE3, PE4, and PE5. In particular, the PE4 editing system consists of a prime editor enzyme (nickase Cas9-RT fusion), MLH1dn, and pegRNA. The PE5 editing system consists of a prime editor enzyme, MLH1dn, pegRNA, and second-strand nicking sgRNA.

FIG. 156 shows prime editing at CDKL5 in wild-type HeLa and HEK293T cells. The CDKL5 edit is at a site for which the c.1412delA mutation causes CDKL5 deficiency disorder. epegRNAs were used for editing the CDKL5 locus. Bars represent the mean of n=3 independent biological replicates.

FIG. 157 shows correction of CDKL5 c.1412delA via an A•T insertion and a silent G•C-to-A•T edit in iPSCs derived from a patient heterozygous for the allele. Editing efficiencies indicate the percentage of sequencing reads with c.1412delA correction out of editable alleles that carry the mutation. Indel frequencies reflect all sequencing reads that contain any indels. Bars represent the mean of n=3 independent biological replicates.

FIG. 158 shows the correction of CDKL5 c.1412delA via an A•T insertion and a G•C-to-A•T edit in iPSCs derived from a patient heterozygous for the disease allele. Editing efficiencies indicate the percentage of sequencing reads with c.1412delA correction out of editable alleles that carry the mutation. Indel frequencies reflect all sequencing reads that contain any indels that do not map to the c.1412delA allele or wild-type sequence. 1 μg of PE2 mRNA was used in all conditions shown. Bars represent the mean of n=3 independent biological replicates.

FIG. 159 shows the combination of MLH1dn and epegRNAs for CDKL5 editing. The editing efficiency of a CDKL5 c.1412 A to G mutation in HEK293T cells is shown.

FIG. 160 shows optimization of the nicking sgRNA for prime editing at CDKL5. The editing efficiency of installation of a CDKL5 silent +1 C to T mutation (c.1412delA site) in HEK293T cells is shown.

FIG. 161 shows that SpCas9-PE can generate indel byproducts when editing wild type CDKL5.

FIG. 162 shows that NRCH SpCas9 variant prime editors do not generate indel byproducts when editing wild type CDKL5.

FIG. 163 shows that NRTH SpCas9 variant prime editors do not generate indel byproducts when editing wild type CDKL5.

FIG. 164 shows optimization of pegRNAs for installation of a nucleotide transition at c.1412 in the CDKL5 gene in HEK293T cells using PE2.

FIG. 165 shows screening of nicking guides used in PE3-mediated editing at c.1412. All guides contain the optimal PBS and template lengths identified in FIG. 164 and encode a +1 G-A transition. CDKL5h37 is a pegRNA, and the remaining guides are all epegRNAs that contain different RNA structural motifs 3′ of the PBS via an 8-nucleotide linker. CDKL5h37 and JNpeg953 showed the highest editing efficiency.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Aptamer

An “aptamer” refers to an oligonucleotide or peptide molecule that binds to a specific target molecule. Aptamers include DNA or RNA aptamers that are short single-stranded DNA- or RNA-based oligonucleotides that can selectively bind to small molecular ligands or protein targets with high affinity and specificity, when folded into their unique three-dimensional structures. On the molecular level, aptamers bind to its cognate target through various non-covalent interactions, electrostatic interactions, hydrophobic interactions, and induced fitting. Further reference can be made to Ku et al., “Nucleic Acid Aptamers: An Emerging Tool for Biotechnology and Biomedical Sensing,” Sensors, 2015, 15(7): 16281-16313. The present disclosure contemplates the use of any aptamer, including those obtained from commercial sources. For example, numerous aptamers may be obtained from APTAGEN (www.aptagen.com) and include, but are not limited to, thrombin (15mer), HIV-1 TAR RNA hairpin loop (B22-19), human immunoglobulin G (IgG) (Apt 8), reactive green 19 (GR-30), abrin toxin (TA6), malachite green (MG-4), PSMA aptamer (A10-3), tenascin-C (GBI-10), and methylenedianiline (M1). Another example is prequeosine₁-1 riboswitch aptamer-one of the smallest natural tertiary RNA structures (also known as evopreQ₁-1).

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 37). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 37). In some embodiments, the Cas9 variant comprises a fragment of SEQ ID NO: 37 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 37). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 37).

cDNA

The term “cDNA” refers to a strand of DNA copied from an RNA template. cDNA is complementary to the RNA template.

### Circular Permutant

As used herein, the term “circular permutant” refers to a protein or polypeptide (e.g., a Cas9) comprising a circular permutation, which is a change in the protein's structural configuration involving a change in the order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques.

### Circularly Permuted Cas9

The term “circularly permuted Cas9” refers to any Cas9 protein, or variant thereof, that occurs as a circular permutant, whereby its N- and C-termini have been topically rearranged. Such circularly permuted Cas9 proteins (“CP-Cas9”), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of which are incorporated herein by reference. The instant disclosure contemplates any previously known CP-Cas9 or use of a new CP-Cas9 so long as the resulting circularly permuted protein retains the ability to bind DNA when complexed with a guide RNA (gRNA). Exemplary CP-Cas9 proteins are SEQ ID NOs: 88-97.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a pegRNA that is utilized as a template strand by a polymerase of a prime editor to encode a 3′ single-strand DNA flap that contains the desired edit and which then, through the mechanism of prime editing, replaces the corresponding endogenous strand of DNA at the target site. In various embodiments, the DNA synthesis template is shown in FIG. 3A (in the context of a pegRNA comprising a 5′ extension arm), FIG. 3B (in the context of a pegRNA comprising a 3′ extension arm), FIG. 3C (in the context of an internal extension arm), FIG. 3D (in the context of a 3′ extension arm), and FIG. 3E (in the context of a 5′ extension arm). The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments (e.g., as depicted in FIGS. 3D-3E), the DNA synthesis template comprises an the “edit template” and a “homology arm.” In various embodiments (e.g., as depicted in FIGS. 3D-3E), the DNA synthesis template (4) may comprise the “edit template” and a “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region, as well. Said another way, in the case of a 3′ extension arm, the DNA synthesis template (3) can include the portion of the extension arm (3) that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template (3) can include the portion of the extension arm that spans from the 5′ end of the pegRNA molecule to the 3′ end of the edit template. In some embodiments, the DNA synthesis template excludes the primer binding site (PBS) of pegRNAs either having a 3′ extension arm or a 5′ extension arm. Certain embodiments described here (e.g., FIG. 71A) refer to an “RT template,” which is inclusive of the edit template and the homology arm, i.e., the sequence of the pegRNA extension arm which is actually used as a template during DNA synthesis. The term “RT template” is equivalent to the term “DNA synthesis template.” In certain embodiments, an RT template may be used to refer to a template polynucleotide for reverse transcription, e.g., in a prime editing system, complex or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent polymerization, e.g., in a prime editing system, complex or method using a prime editor having a polymerase that is an RNA-dependent DNA polymerase or a DNA-dependent DNA polymerase.

In the case of trans prime editing (e.g., FIG. 3G and FIG. 3H), the primer binding site (PBS) and the DNA synthesis template can be engineered into a separate molecule referred to as a trans prime editor RNA template (tPERT).

In some embodiments, the DNA synthesis template is a single-stranded portion of the PEgRNA that is 5′ of the PBS and comprises a region of complementarity to the PAM strand (i.e., the non-target strand or the edit strand), and comprises one or more nucleotide edits compared to the endogenous sequence of the double stranded target DNA. In some embodiments, the DNA synthesis template is complementary or substantially complementary to a sequence on the non-target strand that is downstream of a nick site, except for one or more non-complementary nucleotides at the intended nucleotide edit positions. In some embodiments, the DNA synthesis template is complementary or substantially complementary to a sequence on the non-target strand that is immediately downstream (i.e., directly downstream) of a nick site, except for one or more non-complementary nucleotides at the intended nucleotide edit positions. In some embodiments, one or more of the non-complementary nucleotides at the intended nucleotide edit positions are immediately downstream of a nick site. In some embodiments, the DNA synthesis template comprises one or more nucleotide edits relative to the double-stranded target DNA sequence. In some embodiments, the DNA synthesis template comprises one or more nucleotide edits relative to the non-target strand of the double-stranded target DNA sequence. For each PEgRNA described herein, a nick site is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence). In some embodiments, the DNA synthesis template and the primer binding site are immediately adjacent to each other. The terms “nucleotide edit”, “nucleotide change”, “desired nucleotide change”, and “desired nucleotide edit” are used interchangeably to refer to a specific nucleotide edit, e.g., a specific deletion of one or more nucleotides, a specific insertion of one or more nucleotides, a specific substitution(s) of one or more nucleotides, or a combination thereof, at one a specific position in a DNA synthesis template of a PEgRNA to be incorporated in a target DNA sequence. In some embodiments, the DNA synthesis template comprises more than one nucleotide edits relative to the double-stranded target DNA sequence. In such embodiments, each nucleotide edit is a specific nucleotide edit at a specific position in the DNA synthesis template, each nucleotide edit is at a different specific position relative to any of the other nucleotide edits in the DNA synthesis template, and each nucleotide edit is independently selected from a specific deletion of one or more nucleotides, a specific insertion of one or more nucleotides, a specific substitution(s) of one or more nucleotides, or a combination thereof. A nucleotide edit may refer to the edit on the DNA synthesis template as compared to the sequence on the target strand of the target gene, or may refer to the edit encoded by the DNA synthesis template on the newly synthesized single stranded DNA that replaces the endogenous target DNA sequence on the non-target strand, in either case, may be refer to as a nucleotide edit compared to the target DNA sequence.

### Downstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Edit Template

The term “edit template” refers to a portion of the extension arm that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here (e.g., FIG. 71A) refer to “an RT template,” which refers to both the edit template and the homology arm together, i.e., the sequence of the pegRNA extension arm which is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Effective Amount

The term “effective amount,” as used herein, refers to an amount of a biologically active agent that is sufficient to elicit a desired biological response. For example, in some embodiments, an effective amount of a prime editor (PE) may refer to the amount of the editor that is sufficient to edit a target site nucleotide sequence, e.g., a genome. In some embodiments, an effective amount of a prime editor (PE) provided herein, e.g., of a fusion protein comprising a nickase Cas9 domain and a reverse transcriptase may refer to the amount of the fusion protein that is sufficient to induce editing of a target site specifically bound and edited by the fusion protein. As will be appreciated by the skilled artisan, the effective amount of an agent, e.g., a fusion protein, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Error-Prone Reverse Transcriptase

As used herein, the term “error-prone” reverse transcriptase (or more broadly, any polymerase) refers to a reverse transcriptase (or more broadly, any polymerase) that occurs naturally or which has been derived from another reverse transcriptase (e.g., a wild type M-MLV reverse transcriptase) which has an error rate that is less than the error rate of wild type M-MLV reverse transcriptase. The error rate of wild type M-MLV reverse transcriptase is reported to be in the range of one error in 15,000 (higher) to 27,000 (lower). An error rate of 1 in 15,000 corresponds with an error rate of 6.7×10⁻⁵. An error rate of 1 in 27,000 corresponds with an error rate of 3.7×10⁻⁵. See Boutabout et al. (2001) “DNA synthesis fidelity by the reverse transcriptase of the yeast retrotransposon Ty1,” Nucleic Acids Res 29(11):2217-2222, which is incorporated herein by reference. Thus, for purposes of this application, the term “error prone” refers to those RT that have an error rate that is greater than one error in 15,000 nucleobase incorporation (6.7×10⁻⁵ or higher), e.g., 1 error in 14,000 nucleobases (7.14×10⁻⁵ or higher), 1 error in 13,000 nucleobases or fewer (7.7×10⁻⁵ or higher), 1 error in 12,000 nucleobases or fewer (7.7×10⁻⁵ or higher), 1 error in 11,000 nucleobases or fewer (9.1×10⁻⁵ or higher), 1 error in 10,000 nucleobases or fewer (1×10⁻⁴ or 0.0001 or higher), 1 error in 9,000 nucleobases or fewer (0.00011 or higher), 1 error in 8,000 nucleobases or fewer (0.00013 or higher) 1 error in 7,000 nucleobases or fewer (0.00014 or higher), 1 error in 6,000 nucleobases or fewer (0.00016 or higher), 1 error in 5,000 nucleobases or fewer (0.0002 or higher), 1 error in 4,000 nucleobases or fewer (0.00025 or higher), 1 error in 3,000 nucleobases or fewer (0.00033 or higher), 1 error in 2,000 nucleobase or fewer (0.00050 or higher), or 1 error in 1,000 nucleobases or fewer (0.001 or higher), or 1 error in 500 nucleobases or fewer (0.002 or higher), or 1 error in 250 nucleobases or fewer (0.004 or higher).

### Extein

The term “extein,” as used herein, refers to a polypeptide sequence that is flanked by an intein and is ligated to another extein during the process of protein splicing to form a mature, spliced protein. Typically, an intein is flanked by two extein sequences that are ligated together when the intein catalyzes its own excision. Exteins, accordingly, are the protein analog to exons found in mRNA. For example, a polypeptide comprising an intein may be of the structure extein(N)—intein—extein(C). After excision of the intein and splicing of the two exteins, the resulting structures are extein(N)—extein(C) and a free intein. In various configurations, the exteins may be separate proteins (e.g., half of a Cas9 or Prime editor), each fused to a split-intein, wherein the excision of the split inteins causes the splicing together of the extein sequences.

### Extension Arm

The term “extension arm” refers to a nucleotide sequence component of a pegRNA which comprises a primer binding site and a DNA synthesis template (e.g., an edit template and a homology arm) for a polymerase (e.g., a reverse transcriptase). In some embodiments, e.g., FIG. 3D, the extension arm is located at the 3′ end of the guide RNA. In other embodiments, e.g., FIG. 3E, the extension arm is located at the 5′ end of the guide RNA. In some embodiments, the extension arm comprises a DNA synthesis template and a primer binding site. In some embodiments, the extension arm comprises the following components in a 5′ to 3′ direction: the DNA synthesis template, and the primer binding site. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand.

Further details, such as the length of the extension arm, are described elsewhere herein.

The extension arm may also be described as comprising generally two regions: a primer binding site (PBS) and a DNA synthesis template, as shown in FIG. 3G (top), for instance. The primer binding site binds to the primer sequence that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the pegRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and which ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the pegRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Flap Endonuclease (e.g., FEN1)

As used herein, the term “flap endonuclease” refers to an enzyme that catalyzes the removal of 5′ single strand DNA flaps. These are enzymes that process the removal of 5′ flaps formed during cellular processes, including DNA replication. The prime editing methods herein described may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519, Tsutakawa et al., “Human flap endonuclease structures, DNA double-base flipping, and a unified understanding of the FEN1 superfamily,” Cell, 2011, 145(2): 198-211, and Balakrishnan et al., “Flap Endonuclease 1,” Annu Rev Biochem, 2013, Vol 82: 119-138 (each of which are incorporated herein by reference). An exemplary flap endonuclease is FEN1, which can be represented by the following amino acid sequence:

| DE- | | SEQ ID |
|---|---|---|
| SCRIPTION | SEQUENCE | NO: |
| FEN1 | MGIQGLAKLIADVAPSAIRENDIKSYFGRKVAIDA | SEQ ID |
| WILD | SMSIYQFLIAVRQGGDVLQNEEGETTSHLMGMFYR | NO: 15 |
| TYPE | TIRMMENGIKPVYVFDGKPPQLKSGELAKRSERRA | |
| | EAEKQLQQAQAAGAEQEVEKFTKRLVKVTKQHNDE | |
| | CKHLLSLMGIPYLDAPSEAEASCAALVKAGKVYAA | |
| | ATEDMDCLTFGSPVLMRHLTASEAKKLPIQEFHLS | |
| | RILQELGLNQEQFVDLCILLGSDYCESIRGIGPKR | |
| | AVDLIQKHKSIEEIVRRLDPNKYPVPENWLHKEAH | |
| | QLFLEPEVLDPESVELKWSEPNEEELIKFMCGEKQ | |
| | FSEERIRSGVKRLSKSRQGSTQGRLDDFFKVTGSL | |
| | SSAKRKEPEPKGSTKKKAKTGAAGKFKRGK | |

### Functional Equivalent

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, mutated, or synthetic version of protein X which bears an equivalent function.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Gene of Interest (GOI)

The term “gene of interest” or “GOI” refers to a gene that encodes a biomolecule of interest (e.g., a protein or an RNA molecule). A protein of interest can include any intracellular protein, membrane protein, or extracellular protein, e.g., a nuclear protein, transcription factor, nuclear membrane transporter, intracellular organelle associated protein, a membrane receptor, a catalytic protein, and enzyme, a therapeutic protein, a membrane protein, a membrane transport protein, a signal transduction protein, or an immunological protein (e.g., an IgG or other antibody protein), etc. The gene of interest may also encode an RNA molecule, including, but not limited to, messenger RNA (mRNA), transfer RNA (tRNA), ribosomal RNA (rRNA), small nuclear RNA (snRNA), antisense RNA, guide RNA, microRNA (miRNA), small interfering RNA (siRNA), and cell-free RNA (cfRNA).

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “pegRNAs”) which have been invented for the prime editing methods and composition disclosed herein.

Guide RNAs or pegRNAs may comprise various structural elements that include, but are not limited to:

Spacer sequence—the sequence in the guide RNA or pegRNA (having about 20 nts in length) which binds to the protospacer in the target DNA.

gRNA core (or gRNA scaffold or backbone sequence)—refers to the sequence within the gRNA that is responsible for Cas9 binding, it does not include the 20 bp spacer/targeting sequence that is used to guide Cas9 to target DNA.

Extension arm—a single strand extension at the 3′ end or the 5′ end of the pegRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the guide RNA or pegRNA may comprise a transcriptional termination sequence at the 3′ of the molecule.

### G-Quadruplex

The term “G-quadruplex” refers to its ordinary and customary meaning. A G-quadruplex is a complex three-dimensional nucleic acid moiety formed in nucleic acid sequences that are rich in guanine (G). They are helical in shape and formed from interconnected stacks of guanine tetrads (or “G-tetrads”), which individually are flat, ring-shaped structures formed from four guanines, and which can be stabilized by the presence of a cation (e.g., potassium) which sits in a central channel between pairs of G-tetrads. G-quadruplexes are a diverse collection of structures and not a single structure. Further reference to G-quadruplexes can be found in (1) Kwok et al., “G-Quadruplexes: Prediction, Characterization, and Biological Application,” Trends in Biotechnology, 2017, Vol. 35(10; pp. 997-1013; (2) Hansel-Hertsch R. et al., “DNA G-quadruplexes in the human genome: detection, functions and therapeutic potential,” Nat. Rev. Mol. Cell Biol., 2017; 18: 279-284; and (3) Millevoi S. et al., “G-quadruplexes in RNA biology,” Wiley Interdiscip. Rev. RNA., 2012; 3: 495-507, each of which are incorporated herein by reference.

### Homology Arm

The term “homology arm” refers to a portion of the extension arm that encodes a portion of the resulting reverse transcriptase-encoded single strand DNA flap that is to be integrated into the target DNA site by replacing the endogenous strand. The portion of the single strand DNA flap encoded by the homology arm is complementary to the non-edited strand of the target DNA sequence, which facilitates the displacement of the endogenous strand and annealing of the single strand DNA flap in its place, thereby installing the edit. This component is further defined elsewhere. The homology arm is part of the DNA synthesis template since it is by definition encoded by the polymerase of the prime editors described herein.

### Host Cell

The term “host cell,” as used herein, refers to a cell that can host, replicate, and express a vector described herein, e.g., a vector comprising a nucleic acid molecule encoding a fusion protein comprising a Cas9 or Cas9 equivalent and a reverse transcriptase.

### Inteins

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.” Inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Ligand-Dependent Intein

The term “ligand-dependent intein,” as used herein refers to an intein that comprises a ligand-binding domain. Typically, the ligand-binding domain is inserted into the amino acid sequence of the intein, resulting in a structure intein (N)—ligand-binding domain—intein (C). Typically, ligand-dependent inteins exhibit no or only minimal protein splicing activity in the absence of an appropriate ligand, and a marked increase of protein splicing activity in the presence of the ligand. In some embodiments, the ligand-dependent intein does not exhibit observable splicing activity in the absence of ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art, and in include those provided below and those described in published U.S. Patent Application U.S. 2014/0065711 A1; Mootz et al., “Protein splicing triggered by a small molecule.” J. Am. Chem. Soc. 2002; 124, 9044-9045; Mootz et al., “Conditional protein splicing: a new tool to control protein structure and function in vitro and in vivo.” J. Am. Chem. Soc. 2003; 125, 10561-10569; Buskirk et al., Proc. Natl. Acad. Sci. USA. 2004; 101, 10505-10510); Skretas & Wood, “Regulation of protein activity with small-molecule-controlled inteins.” Protein Sci. 2005; 14, 523-532; Schwartz, et al., “Post-translational enzyme activation in an animal via optimized conditional protein splicing.” Nat. Chem. Biol. 2007; 3, 50-54; Peck et al., Chem. Biol. 2011; 18 (5), 619-630; the entire contents of each are hereby incorporated by reference. Exemplary sequences are as follows:

| NAME | SEQUENCE OF LIGAND-DEPENDENT INTEIN | SEQ ID NO: |
|---|---|---|
| 2-4 | CLAEGTRIFDPVTGTTHRIEDVVDGRKPIHVVAAAKDGT | SEQ ID NO: 16 |
| INTEIN: | LLARPVVSWFDQGTRDVIGLRIAGGAIVWATPDHKVLTE | |
| | YGWRAAGELRKGDRVAGPGGSGNSLALSLTADQMVSAL | |
| | LDAEPPILYSEYDPTSPFSEASMMGLLTNLADRELVHMIN | |
| | WAKRVPGFVDLTLHDQAHLLECAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEELRYSVIREVLPTRR | |
| | ARTFDLEVEELHTLVAEGVVVHNC | |
| 3-2 | CLAEGTRIFDPVTGTTHRIEDVVDGRKPIHVVAVAKDGTL | SEQ ID NO: 17 |
| INTEIN | LARPVVSWFDQGTRDVIGLRIAGGAIVWATPDHKVLTEY | |
| | GWRAAGELRKGDRVAGPGGSGNSLALSLTADQMVSALL | |
| | DAEPPILYSEYDPTSPFSEASMMGLLTNLADRELVHMIN | |
| | WAKRVPGFVDLTLHDQAHLLECAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYTNVVPLYDLLLEMLDAHRLHAGG | |
| | SGASRVQAFADALDDKFLHDMLAEELRYSVIREVLPTRR | |
| | ARTFDLEVEELHTLVAEGVVVHNC | |
| 30R3-1 | CLAEGTRIFDPVTGTTHRIEDVVDGRKPIHVVAAAKDGT | SEQ ID NO: 18 |
| INTEIN | LLARPVVSWFDQGTRDVIGLRIAGGATVWATPDHKVLTE | |
| | YGWRAAGELRKGDRVAGPGGSGNSLALSLTADQMVSAL | |
| | LDAEPPIPYSEYDPTSPFSEASMMGLLTNLADRELVHMIN | |
| | WAKRVPGFVDLTLHDQAHLLECAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEGLRYSVIREVLPTRR | |
| | ARTFDLEVEELHTLVAEGVVVHNC | |
| 30R3-2 | CLAEGTRIFDPVTGTTHRIEDVVDGRKPIHVVAAAKDGT | SEQ ID NO: 19 |
| INTEIN | LLARPVVSWFDQGTRDVIGLRIAGGATVWATPDHKVLTE | |
| | YGWRAAGELRKGDRVAGPGGSGNSLALSLTADQMVSAL | |
| | LDAEPPILYSEYDPTSPFSEASMMGLLTNLADRELVHMIN | |
| | WAKRVPGFVDLTLHDQAHLLECAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEELRYSVIREVLPTRR | |
| | ARTFDLEVEELHTLVAEGVVVHNC | |
| 30R3-3 | CLAEGTRIFDPVTGTTHRIEDVVDGRKPIHVVAAAKDGT | SEQ ID NO: 20 |
| INTEIN | LLARPVVSWFDQGTRDVIGLRIAGGATVWATPDHKVLTE | |
| | YGWRAAGELRKGDRVAGPGGSGNSLALSLTADQMVSAL | |
| | LDAEPPIPYSEYDPTSPFSEASMMGLLTNLADRELVHMIN | |
| | WAKRVPGFVDLTLHDQAHLLECAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEELRYSVIREVLPTRR | |
| | ARTFDLEVEELHTLVAEGVVVHNC | |
| 37R3-1 | CLAEGTRIFDPVTGTTHRIEDVVDGRKPIHVVAAAKDGT | SEQ ID NO: 21 |
| INTEIN | LLARPVVSWFDQGTRDVIGLRIAGGATVWATPDHKVLTE | |
| | YGWRAAGELRKGDRVAGPGGSGNSLALSLTADQMVSAL | |
| | LDAEPPILYSEYNPTSPFSEASMMGLLTNLADRELVHMIN | |
| | WAKRVPGFVDLTLHDQAHLLERAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEGLRYSVIREVLPTRR | |
| | ARTFDLEVEELHTLVAEGVVVHNC | |
| 37R3-2 | CLAEGTRIFDPVTGTTHRIEDVVDGRKPIHVVAAAKDGT | SEQ ID NO: 22 |
| INTEIN | LLARPVVSWFDQGTRDVIGLRIAGGAIVWATPDHKVLTE | |
| | YGWRAAGELRKGDRVAGPGGSGNSLALSLTADQMVSAL | |
| | LDAEPPILYSEYDPTSPFSEASMMGLLTNLADRELVHMIN | |
| | WAKRVPGFVDLTLHDQAHLLERAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEGLRYSVIREVLPTRR | |
| | ARTFDLEVEELHTLVAEGVVVHNC | |
| 37R3-3 | CLAEGTRIFDPVTGTTHRIEDVVDGRKPIHVVAVAKDGTL | SEQ ID NO: 23 |
| INTEIN | LARPVVSWFDQGTRDVIGLRIAGGATVWATPDHKVLTEY | |
| | GWRAAGELRKGDRVAGPGGSGNSLALSLTADQMVSALL | |
| | DAEPPILYSEYDPTSPFSEASMMGLLTNLADRELVHMIN | |
| | WAKRVPGFVDLTLHDQAHLLERAWLEILMIGLVWRSME | |
| | HPGKLLFAPNLLLDRNQGKCVEGMVEIFDMLLATSSRFR | |
| | MMNLQGEEFVCLKSIILLNSGVYTFLSSTLKSLEEKDHIH | |
| | RALDKITDTLIHLMAKAGLTLQQQHQRLAQLLLILSHIRH | |
| | MSNKGMEHLYSMKYKNVVPLYDLLLEMLDAHRLHAGG | |
| | SGASRVQAFADALDDKFLHDMLAEELRYSVIREVLPTRR | |
| | ARTFDLEVEELHTLVAEGVVVHNC | |

The term “linker,” as used herein, refers to a molecule linking two other molecules or moieties. The linker can be an amino acid sequence in the case of a linker joining two fusion proteins. For example, a Cas9 can be fused to a polymerase (e.g., reverse transcriptase) by an amino acid linker sequence. The linker can also be a nucleotide sequence in the case of joining two nucleotide sequences together. For example, in the instant case, the traditional guide RNA is linked via a spacer or linker nucleotide sequence to the RNA extension of a prime editing guide RNA which may comprise a RT template sequence and an RT primer binding site. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Isolated

“Isolated” means altered or removed from the natural state. For example, a nucleic 20 acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In some embodiments, a gene of interest is encoded by an isolated nucleic acid. As used herein, the term “isolated,” refers to the characteristic of a material as provided herein being removed from its original or native environment (e.g., the natural environment if it is naturally occurring). Therefore, a naturally-occurring polynucleotide or protein or polypeptide present in a living animal is not isolated, but the same polynucleotide or polypeptide, separated by human intervention from some or all of the coexisting materials in the natural system, is isolated. An artificial or engineered material, for example, a non-naturally occurring nucleic acid construct, such as the expression constructs and vectors described herein, are, accordingly, also referred to as isolated. A material does not have to be purified in order to be isolated. Accordingly, a material may be part of a vector and/or part of a composition, and still be isolated in that such vector or composition is not part of the environment in which the material is found in nature.

### MS2 Tagging Technique

In various embodiments (e.g., as depicted in the embodiments of FIGS. 72-73 and in Example 19), the term “MS2 tagging technique” refers to the combination of an “RNA-protein interaction domain” (aka “RNA-protein recruitment domain or protein”) paired up with an RNA-binding protein that specifically recognizes and binds to the RNA-protein interaction domain, e.g., a specific hairpin structure. These types of systems can be leveraged to recruit a variety of functionalities to a prime editor complex that is bound to a target site. The MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of prime editing, the MS2 tagging technique comprises introducing the MS2 hairpin into a desired RNA molecule involved in prime editing (e.g., a pegRNA or a tPERT), which then constitutes a specific interactable binding target for an RNA-binding protein that recognizes and binds to that structure. In the case of the MS2 hairpin, it is recognized and bound by the MS2 bacteriophage coat protein (MCP). And, if MCP is fused to another protein (e.g., a reverse transcriptase or other DNA polymerase), then the MS2 hairpin may be used to “recruit” that other protein in trans to the target site occupied by the prime editing complex.

The prime editors described herein may incorporate as an aspect any known RNA-protein interaction domain to recruit or “co-localize” specific functions of interest to a prime editor complex. A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 24).

The amino acid sequence of the MCP or MS2cp is: GSASNFTQFVLVDNGGTGDVTVAPSNFANGVAEWISSNSRSQAYKVTCSVRQSSAQ NRKYTIKVEVPKVATQTVGGEELPVAGWRSYLNMELTIPIFATNSDCELIVKAMQGL LKDGNPIPSAIAANSGIY (SEQ ID NO: 25).

The MS2 hairpin (or “MS2 aptamer”) may also be referred to as a type of “RNA effector recruitment domain” (or equivalently as “RNA-binding protein recruitment domain” or simply as “recruitment domain”) since it is a physical structure (e.g., a hairpin) that is installed into a pegRNA or tPERT that effectively recruits other effector functions (e.g., RNA-binding proteins having various functions, such as DNA polymerases or other DNA-modifying enzymes) to the pegRNA or rPERT that is so modified, and thus, co-localizing effector functions in trans to the prime editing machinery. This application is not intended to be limited in any way to any particular RNA effector recruitment domains and may include any available such domain, including the MS2 hairpin. Example 19 and FIG. 72(b) depicts the use of the MS2 aptamer joined to a DNA synthesis domain (i.e., the tPERT molecule) and a prime editor that comprises an MS2cp protein fused to a PE2 to cause the co-localization of the prime editor complex (MS2cp-PE2:sgRNA complex) bound to the target DNA site and the DNA synthesis domain of the tPERT molecule to effectuate the

napDNAbp

As used herein, the term “nucleic acid programmable DNA binding protein” or “napDNAbp,” of which Cas9 is an example, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in a HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical Cas9 sequence or to an equivalent amino acid position in other Cas9 variants or Cas9 equivalents. In some embodiments, the nickase is a Cas9 that comprises a H840A, N854A, and/or N863A mutation relative to a canonical Cas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase. Nuclear localization sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, a NLS comprises the amino acid sequence PKKKRKV (SEQ ID NO: 26) or MDSLLMNRRKFLYQFKNVRWAKGRRETYLC (SEQ ID NO: 27).

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Nucleotide Structural Motifs (or Nucleic Acid Moiety)

As used herein, the term “nucleotide structural motif” or equivalently, “nucleic acid moiety,” refers to nucleic acid molecule or a portion thereof, which forms a secondary or tertiary structure due to basepairing interactions within a single nucleic acid polymer or between two or more nucleic acid polymers. Such nucleotide structural motifs can be formed from DNA, RNA, or a hybrid of DNA and RNA. The term is not meant to refer to standard DNA double-helices. Examples of nucleic acid moieties include, but are not limited to, a toe-loop, hairpin, stem-loop, pseudoknot, aptamer, G quadraplex, tRNA, ribozyme, riboswitch, A-form DNA, B-form DNA, or Z-form DNA.

pegRNA

As used herein, the terms “prime editing guide RNA” or “pegRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein the “primer binding site” comprises a sequence that hybridizes to a single-strand DNA sequence having a 3′ end generated from the nicked DNA of the R-loop.

In certain embodiments, the pegRNAs are represented by FIG. 3A, which shows a pegRNA having a 5′ extension arm, a spacer, and a gRNA core. The 5′ extension further comprises in the 5′ to 3′ direction a reverse transcriptase template, a primer binding site, and a linker. As shown, the reverse transcriptase template may also be referred to more broadly as the “DNA synthesis template” where the polymerase of a prime editor described herein is not an RT, but another type of polymerase.

In certain other embodiments, the pegRNAs are represented by FIG. 3B, which shows a pegRNA having a 5′ extension arm, a spacer, and a gRNA core. The 5′ extension further comprises in the 5′ to 3′ direction a reverse transcriptase template, a primer binding site, and a linker. As shown, the reverse transcriptase template may also be referred to more broadly as the “DNA synthesis template” where the polymerase of a prime editor described herein is not an RT, but another type of polymerase.

In still other embodiments, the pegRNAs are represented by FIG. 3D, which shows a pegRNA having in the 5′ to 3′ direction a spacer (1), a gRNA core (2), and an extension arm (3). The extension arm (3) is at the 3′ end of the pegRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the pegRNA may comprise a transcriptional terminator sequence. These sequence elements of the pegRNAs are further described and defined herein.

In still other embodiments, the pegRNAs are represented by FIG. 3E, which shows a pegRNA having in the 5′ to 3′ direction an extension arm (3), a spacer (1), and a gRNA core (2). The extension arm (3) is at the 5′ end of the pegRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The pegRNAs may also comprise a transcriptional terminator sequence at the 3′ end. These sequence elements of the pegRNAs are further described and defined herein.

### PE1

As used herein, “PE1” refers to a PE complex comprising a fusion protein comprising Cas9(H840A) and a wild type MMLV RT having the following structure: [NLS]-[Cas9(H840A)]-[linker]-[MMLV_RT(wt)]+a desired pegRNA, wherein the PE fusion has the amino acid sequence of SEQ ID NO: 28.

### PE2

As used herein, “PE2” refers to a PE complex comprising a fusion protein comprising Cas9(H840A) and a variant MMLV RT having the following structure: [NLS]-[Cas9(H840A)]-[linker]-[MMLV_RT(D200N)(T330P)(L603W)(T306K)(W313F)]+a desired pegRNA, wherein the PE fusion has the amino acid sequence of SEQ ID NO: 33.

### PE3

As used herein, “PE3” refers to PE2 plus a second-strand nicking guide RNA that complexes with the PE2 and introduces a nick in the non-edited DNA strand in order to induce preferential replacement of the edited strand.

### PE3b

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE4

As used herein, “PE4” refers to a system comprising PE2 plus an MLH1 dominant negative protein (i.e., wild-type MLH1 with amino acids 754-756 truncated, which may be referred to herein as “MLH1 Δ754-756” or “MLH1dn”) expressed in trans. In some embodiments, PE4 refers to a fusion protein comprising PE2 and an MLH1 dominant negative protein joined via an optional linker.

### PE5

As used herein, “PE5” refers to a system comprising PE3 plus an MLH1 dominant negative protein (i.e., wild-type MLH1 with amino acids 754-756 truncated as described further herein, which may be referred to as “MLH1 Δ754-756” or “MLH1dn”) expressed in trans. In some embodiments, PE5 refers to a fusion protein comprising PE3 and an MLH1 dominant negative protein joined via an optional linker.

### PE-Short

As used herein, “PE-short” refers to a PE construct that is fused to a C-terminally truncated reverse transcriptase, and has the following amino acid sequence:

(SEQ ID NO: 35)
MKRTADGSEFESPKKKRKVDKKYSIGLDIGTNSVGWAVITDEYKVPSKKFK
VLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKE
HPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETR
IDLSQLGGDSGGSSGGSSGSETPGTSESATPESSGGSSGGSSTLNIEDEYR
LHETSKEPDVSLGSTWLSDFPQAWAETGGMGLAVRQAPLIIPLKATSTPVS
IKQYPMSQEARLGIKPHIQRLLDQGILVPCQSPWNTPLLPVKKPGTNDYRP
VQDLREVNKRVEDIHPTVPNPYNLLSGLPPSHQWYTVLDLKDAFFCLRLHP
TSQPLFAFEWRDPEMGISGQLTWTRLPQGFKNSPTLFNEALHRDLADFRIQ
HPDLILLQYVDDLLLAATSELDCQQGTRALLQTLGNLGYRASAKKAQICQK
QVKYLGYLLKEGQRWLTEARKETVMGQPTPKTPRQLREFLGKAGFCRLFIP
GFAEMAAPLYPLTKPGTLFNWGPDQQKAYQEIKQALLTAPALGLPDLTKPF
ELFVDEKQGYAKGVLTQKLGPWRRPVAYLSKKLDPVAAGWPPCLRMVAAIA
VLTKDAGKLTMGQPLVILAPHAVEALVKQPPDRWLSNARMTHYQALLLDTD
RVQFGPVVALNPATLLPLPEEGLQHNCLDNSRLINSGGSKRTADGSEFEPK
KKRKV
KEY:
NUCLEAR LOCALIZATION SEQUENCE (NLS)
TOP: (SEQ ID NO: 29), BOTTOM: (SEQ ID NO: 30)
CAS9(H840A) (SEQ ID NO: 31)
33-AMINO ACID LINKER 1 (SEQ ID NO: 11)
M-MLV TRUNCATED REVERSE TRANSCRIPTASE
(SEQ ID NO: 36)

### Peptide Tag

The term “peptide tag” refers to a peptide amino acid sequence that is genetically fused to a protein sequence to impart one or more functions onto the proteins that facilitate the manipulation of the protein for various purposes, such as, visualization, purification, solubilization, and separation, etc. Peptide tags can include various types of tags categorized by purpose or function, which may include “affinity tags” (to facilitate protein purification), “solubilization tags” (to assist in proper folding of proteins), “chromatography tags” (to alter chromatographic properties of proteins), “epitope tags” (to bind to high affinity antibodies), “fluorescence tags” (to facilitate visualization of proteins in a cell or in vitro).

### Polymerase

As used herein, the term “polymerase” refers to an enzyme that synthesizes a nucleotide strand and which may be used in connection with the prime editor systems described herein. The polymerase can be a “template-dependent” polymerase (i.e., a polymerase which synthesizes a nucleotide strand based on the order of nucleotide bases of a template strand). The polymerase can also be a “template-independent” polymerase (i.e., a polymerase which synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a pegRNA), and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to a novel approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Certain embodiments of prime editing are described in the embodiments of FIGS. 1A-1H and FIG. 72(a)-72(c), among other figures.

Prime editing represents an entirely new platform for genome editing that is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same (or is homologous to) sequence as the endogenous strand (immediately downstream of the nick site) of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility (e.g., as depicted in various embodiments of FIGS. 1A-1F). TPRT is naturally used by mobile DNA elements, such as mammalian non-LTR retrotransposons and bacterial Group II introns^{28,29}. The inventors have herein used Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, where ever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., pegRNA) containing a spacer sequence that anneals to a complementary protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (pegRNA). In reference to FIG. 1G, the prime editing guide RNA (pegRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/pegRNA complex contacts the DNA molecule and the extended pegRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to C pegRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the pegRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking,” as exemplified in FIG. 1F. This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using prime editing described herein, including, but not limited to the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the pegRNA constitutes a single molecule comprising a guide RNA (which itself comprises a spacer sequence and a gRNA core or scaffold) and a 5′ or 3′ extension arm comprising the primer binding site and a DNA synthesis template (e.g., see FIG. 3D, the pegRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer). See FIG. 3G and FIG. 3H as an example of a tPERT that may be used with prime editing.

### Prime Editor

The term “prime editor” refers to the herein described fusion constructs comprising a napDNAbp (e.g., Cas9 nickase) and a reverse transcriptase and is capable of carrying out prime editing on a target nucleotide sequence in the presence of a pegRNA (or “extended guide RNA”). The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a napDNAbp), a pegRNA, and a regular guide RNA capable of directing the second-site nicking step of the non-edited strand as described herein. In other embodiments, the reverse transcriptase component of the “primer editor” may be provided in trans.

### Primer Binding Site

The term “primer binding site” or “the PBS” refers to the portion of nucleotide sequence located on a pegRNA as component of the extension arm (typically for example, at the 3′ end of the extension arm). The term “primer binding site” refers to a single-stranded portion of the PEgRNA as a component of the extension arm that comprises a region of complementarity to a sequence on the non-target strand. In some embodiments, the primer binding site is complementary to a region upstream of a nick site in a non-target strand. In some embodiments, the primer binding site is complementary to a region immediately upstream of a nick site in the non-target strand. In some embodiments, the primer binding site is capable of binding to the primer sequence that is formed after nicking of the target sequence by the prime editor. When the prime editor nicks one strand of the target DNA sequence (e.g., by a Cas nickase component of the prime editor), a 3′-ended ssDNA flap is formed, which serves a primer sequence that anneals to the primer binding site on the pegRNA to prime reverse transcription. FIGS. 27 and 28 show embodiments of the primer binding site located on a 3′ and 5′ extension arm, respectively. In some embodiments, the PBS is complementary to or substantially complementary to, and can anneal to a free 3′ end on the non-target strand of the double stranded target DNA at the nick site. In some embodiments, the PBS annealed to the free 3′ end on the non-target strand can initiate target-primed DNA synthesis.

### Promoter

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In some embodiments, in order for a Cas nickase component of the prime editor to function, it also requires a specific protospacer adjacent motif (PAM), which varies depending on the Cas protein component itself, e.g., the type of Cas protein and the bacterial species from which it is derived. For example, the most commonly used Cas9 nuclease, derived from S. pyogenes, recognizes a PAM sequence of NGG that is directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 37, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases which are error-prone, i.e., which may be referred to as error-prone reverse transcriptases or reverse transcriptases which do not support high fidelity incorporation of nucleotides during polymerization. During synthesis of the single-strand DNA flap based on the RT template integrated with the guide RNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes which are error-prone in their DNA polymerization activity.

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### Protein Splicing

The term “protein splicing,” as used herein, refers to a process in which a sequence, an intein (or split inteins, as the case may be), is excised from within an amino acid sequence, and the remaining fragments of the amino acid sequence, the exteins, are ligated via an amide bond to form a continuous amino acid sequence. The term “trans” protein splicing refers to the specific case where the inteins are split inteins and they are located on different proteins.

### Second-Strand Nicking

The resolution of heteroduplex DNA (i.e., containing one edited and one non-edited strand) formed as a result of prime editing determines long-term editing outcomes. In words, a goal of prime editing is to resolve the heteroduplex DNA (the edited strand paired with the endogenous non-edited strand) formed as an intermediate of PE by permanently integrating the edited strand into the complement, endogenous strand. The approach of “second-strand nicking” can be used herein to help drive the resolution of heteroduplex DNA in favor of permanent integration of the edited strand into the DNA molecule. As used herein, the concept of “second-strand nicking” refers to the introduction of a second nick at a location downstream of the first nick (i.e., the initial nick site that provides the free 3′ end for use in priming of the reverse transcriptase on the extended portion of the guide RNA), preferably on the unedited strand. In certain embodiments, the first nick and the second nick are on opposite strands. In other embodiments, the first nick and the second nick are on opposite strands. In yet another embodiment, the first nick is on the non-target strand (i.e., the strand that forms the single strand portion of the R-loop), and the second nick is on the target strand. In still other embodiments, the first nick is on the edited strand, and the second nick is on the unedited strand. The second nick can be positioned at least 5 nucleotides downstream of the first nick, or at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream of the first nick. The second nick, in certain embodiments, can be introduced between about 5-150 nucleotides on the unedited strand away from the site of the pegRNA-induced nick, or between about 5-140, or between about 5-130, or between about 5-120, or between about 5-110, or between about 5-100, or between about 5-90, or between about 5-80, or between about 5-70, or between about 5-60, or between about 5-50, or between about 5-40, or between about 5-30, or between about 5-20, or between about 5-10. In one embodiment, the second nick is introduced between 14-116 nucleotides away from the pegRNA-induced nick. Without being bound by theory, the second nick induces the cell's endogenous DNA repair and replication processes towards replacement or editing of the unedited strand, thereby permanently installing the edited sequence on both strands and resolving the heteroduplex that is formed as a result of PE. In some embodiments, the edited strand is the non-target strand and the unedited strand is the target strand. In other embodiments, the edited strand is the target strand, and the unedited strand is the non-target strand.

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

In the context of a pegRNA, the first step is the synthesis of a single-strand complementary DNA (i.e., the 3′ ssDNA flap, which becomes incorporated) oriented in the 5′ to 3′ direction which is templated off of the pegRNA extension arm. Whether the 3′ ssDNA flap should be regarded as a sense or antisense strand depends on the direction of transcription since it well accepted that both strands of DNA may serve as a template for transcription (but not at the same time). Thus, in some embodiments, the 3′ ssDNA flap (which overall runs in the 5′ to 3′ direction) will serve as the sense strand because it is the coding strand. In other embodiments, the 3′ ssDNA flap (which overall runs in the 5′ to 3′ direction) will serve as the antisense strand and thus, the template for transcription.

### Spacer Sequence

As used herein, the term “spacer sequence” in connection with a guide RNA or a pegRNA refers to the portion of the guide RNA or pegRNA of about 20 nucleotides (e.g., 16, 17, 18, 19, 20, 21, 22, 23 or 24 nucleotides) which contains a nucleotide sequence that is complementary to the target strand. In some embodiments, the spacer sequence hybridizes to a region on the target strand that is complementary to a protospacer on the non-target strand to form a ssRNA/ssDNA hybrid structure at the target site and a corresponding R loop ssDNA structure of the complementary endogenous DNA strand on the non-target strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Split Intein

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostoc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product, e.g., as shown in FIGS. 66 and 67 with regard to the formation of a complete Prime editor from two separately-expressed halves.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds.

tPERT

See definition for “trans prime editor RNA template (tPERT).”

### Temporal Second-Strand Nicking

As used herein, the term “temporal second-strand nicking” refers to a variant of second strand nicking whereby the installation of the second nick in the unedited strand occurs only after the desired edit is installed in the edited strand. This avoids concurrent nicks on both strands that could lead to double-stranded DNA breaks. The second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, mismatches between the protospacer and the unedited allele should disfavor nicking by the sgRNA until after the editing event on the PAM strand takes place.

### Trans Prime Editing

As used herein, the term “trans prime editing” refers to a modified form of prime editing that utilizes a split pegRNA, i.e., wherein the pegRNA is separated into two separate molecules: an sgRNA and a trans prime editing RNA template (tPERT). The sgRNA serves to target the prime editor (or more generally, to target the napDNAbp component of the prime editor) to the desired genomic target site, while the tPERT is used by the polymerase (e.g., a reverse transcriptase) to write new DNA sequence into the target locus once the tPERT is recruited in trans to the prime editor by the interaction of binding domains located on the prime editor and on the tPERT. In one embodiment, the binding domains can include RNA-protein recruitment moieties, such as a MS2 aptamer located on the tPERT and an MS2cp protein fused to the prime editor. An advantage of trans prime editing is that by separating the DNA synthesis template from the guide RNA, one can potentially use longer length templates.

An embodiment of trans prime editing is shown in FIGS. 3G and 3H. FIG. 3G shows the composition of the trans prime editor complex on the left (“RP-PE:gRNA complex), which comprises an napDNAbp fused to each of a polymerase (e.g., a reverse transcriptase) and a rPERT recruiting protein (e.g., MS2sc), and which is complexed with a guide RNA. FIG. 3G further shows a separate tPERT molecule, which comprises the extension arm features of a pegRNA, including the DNA synthesis template and the primer binding sequence. The tPERT molecule also includes an RNA-protein recruitment domain (which, in this case, is a stem loop structure and can be, for example, MS2 aptamer). As depicted in the process described in FIG. 3H, the RP-PE:gRNA complex binds to and nicks the target DNA sequence. Then, the recruiting protein (RP) recruits a tPERT to co-localize to the prime editor complex bound to the DNA target site, thereby allowing the primer binding site to bind to the primer sequence on the nicked strand, and subsequently, allowing the polymerase (e.g., RT) to synthesize a single strand of DNA against the DNA synthesis template up through the 5′ of the tPERT.

While the tPERT is shown in FIG. 3G and FIG. 3H as comprising the PBS and DNA synthesis template on the 5′ end of the RNA-protein recruitment domain, the tPERT in other configurations may be designed with the PBS and DNA synthesis template located on the 3′ end of the RNA-protein recruitment domain. However, the tPERT with the 5′ extension has the advantage that synthesis of the single strand of DNA will naturally terminate at the 5′ end of the tPERT and thus, does not risk using any portion of the RNA-protein recruitment domain as a template during the DNA synthesis stage of prime editing.

### Transitions

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transversions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversions

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

### 5′ Endogenous DNA Flap

As used herein, the term “5′ endogenous DNA flap” refers to the strand of DNA situated immediately downstream of the PE-induced nick site in the target DNA. The nicking of the target DNA strand by PE exposes a 3′ hydroxyl group on the upstream side of the nick site and a 5′ hydroxyl group on the downstream side of the nick site. The endogenous strand ending in the 3′ hydroxyl group is used to prime the DNA polymerase of the prime editor (e.g., wherein the DNA polymerase is a reverse transcriptase). The endogenous strand on the downstream side of the nick site and which begins with the exposed 5′ hydroxyl group is referred to as the “5′ endogenous DNA flap” and is ultimately removed and replaced by the newly synthesized replacement strand (i.e., “3′ replacement DNA flap”) the encoded by the extension of the pegRNA.

### 5′ Endogenous DNA Flap Removal

As used herein, the term “5′ endogenous DNA flap removal” or “5′ flap removal” refers to the removal of the 5′ endogenous DNA flap that forms when the RT-synthesized single-strand DNA flap competitively invades and hybridizes to the endogenous DNA, displacing the endogenous strand in the process. Removing this endogenous displaced strand can drive the reaction towards the formation of the desired product comprising the desired nucleotide change. The cell's own DNA repair enzymes may catalyze the removal or excision of the 5′ endogenous flap (e.g., a flap endonuclease, such as EXO1 or FEN1). Also, host cells may be transformed to express one or more enzymes that catalyze the removal of said 5′ endogenous flaps, thereby driving the process toward product formation (e.g., a flap endonuclease). Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference).

### 3′ Replacement DNA Flap

As used herein, the term “3′ replacement DNA flap” or simply, “replacement DNA flap,” refers to the strand of DNA that is synthesized by the prime editor and which is encoded by the extension arm of the prime editor pegRNA. More in particular, the 3′ replacement DNA flap is encoded by the polymerase template of the pegRNA. The 3′ replacement DNA flap comprises the same sequence as the 5′ endogenous DNA flap except that it also contains the edited sequence (e.g., single nucleotide change). The 3′ replacement DNA flap anneals to the target DNA, displacing or replacing the 5′ endogenous DNA flap (which can be excised, for example, by a 5′ flap endonuclease, such as FEN1 or EXO1) and then is ligated to join the 3′ end of the 3′ replacement DNA flap to the exposed 5′ hydroxyl end of endogenous DNA (exposed after excision of the 5′ endogenous DNA flap, thereby reforming a phosphodiester bond and installing the 3′ replacement DNA flap to form a heteroduplex DNA containing one edited strand and one unedited strand. DNA repair processes resolve the heteroduplex by copying the information in the edited strand to the complementary strand permanently installs the edit in to the DNA. This resolution process can be driven further to completion by nicking the unedited strand, i.e., by way of “second-strand nicking,” as described herein.

The terms “cleavage site,” “nick site,” and “cut site” as used interchangeably herein in the context of prime editing, refer to a specific position in between two nucleotides or two base pairs in the double-stranded target DNA sequence. In some embodiments, the position of a nick site is determined relative to the position of a specific PAM sequence. In some embodiments, the nick site is the particular position where a nick will occur when the double stranded target DNA is contacted with a napDNAbp, e.g., a nickase such as a Cas nickase, that recognizes a specific PAM sequence. For each PEgRNA described herein, a nick site is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence).

In some embodiments, a nick site is in a target strand of the double-stranded target DNA sequence. In some embodiments, a nick site is in a non-target strand of the double-stranded target DNA sequence. In some embodiments, the nick site is in a protospacer sequence. In some embodiments, the nick site is adjacent to a protospacer sequence. In some embodiments, a nick site is downstream of a region, e.g., on a non-target strand, that is complementary to a primer binding site of a PEgRNA. In some embodiments, a nick site is downstream of a region, e.g., on a non-target strand, that binds to a primer binding site of a PEgRNA. In some embodiments, a nick site is immediately downstream of a region, e.g., on a non-target strand, that is complementary to a primer binding site of a PEgRNA. In some embodiments, the nick site is upstream of a specific PAM sequence on the non-target strand of the double stranded target DNA, wherein the PAM sequence is specific for recognition by a napDNAbp that associates with the gRNA core of a PEgRNA. In some embodiments, the nick site is downstream of a specific PAM sequence on the non-target strand of the double stranded target DNA. wherein the PAM sequence is specific for recognition by a napDNAbp that associates with the gRNA core of a PEgRNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

### DETAILED DESCRIPTION OF CERTAIN EMBODIMENTS

The present disclosure provides next-generation modified pegRNAs with improved properties, including but not limited to, increased stability, increased lifespan in vivo, and/or improved binding affinity for a napDNAbp. These modified pegRNAs result in improved activity and/or efficiency of prime editing when used in conjunction with a prime editor, such as a fusion protein comprising a Cas9 nickase domain and a reverse transcriptase domain. In particular, the inventors have discovered that pegRNAs may suffer from various deficiencies, including reduced affinity to a nucleic acid programmable DNA binding protein (e.g., a Cas9 nickase), increased susceptibility to degradation relative to canonical single guide RNAs (sgRNAs) (in particular, degradation of the extension arm), and tendency toward inactivation due to unwanted duplex formation between the extension arm (and specifically, the primer binding site of the extension arm) and the spacer sequence in the pegRNA, thereby competing against the binding of the pegRNA's spacer and primer binding site to the strands of a target DNA. Without being bound by theory, these issues arise because of the presence of the extension arm feature that is an integral part of the pegRNA which is not present in canonical sgRNAs. To overcome these deficiencies, the present inventors have discovered that pegRNAs may be modified in one or more several ways to improve their overall stability and/or performance in prime editing. First, the inventors have discovered that appending one or more RNA structural motif's to a pegRNA can protect against degradation of the pegRNA. Such RNA structural motifs can include, but are not limited to (i) a prequeosine1-1 riboswitch aptamer (evopreQ1) and variants thereof, (ii) a frameshifting pseudoknot from Moloney murine leukemia virus (MMLV)22, hereafter referred to as “mpknot,” and variants thereof (iii) G-quadruplexes, (iv) hairpin structures (e.g., 15-bp hairpins), (v) xrRNA, and (vi) a P4-P6 domain of the group I intron. Second, the inventors have discovered various ways to reduce the formation of a duplex between the primer binding site (PBS) of the extension arm and the spacer sequence of the pegRNA (i.e., reducing the PBS/spacer binding interaction). In one embodiment, PBS/spacer binder interaction is avoided by stabilizing the 3′ extension arm, including but not limited to (i) occluding the PBS with toeholds that dissociate upon napDNAbp (e.g., Cas9 nickase) binding, (ii) providing the 3′ extension arm in trans, i.e., moving the 3′ extension arm or portion thereof (e.g, PBS and/or PBS and the DNA template portions) from the pegRNA to another molecule, e.g., the nicking gRNA, and (iii) introduction of chemical modifications to pegRNA that favor RNA/DNA duplex formation but disfavor RNA/RNA duplex formation, thereby promoting the desired interaction between the PBS of the pegRNA and the target DNA. Collectively, the modified pegRNAs disclosed herein resulting from the implementation of these strategies are referred to herein as “engineered” pegRNAs or “epegRNAs” or equivalently as “modified pegRNAs.”

In addition, the disclosure provides prime editing complexes comprising a prime editor complexed with an engineered pegRNA disclosed herein, as well as to nucleotide sequences and expression vectors encoding said engineered pegRNAs and prime editing complexes comprising the engineered pegRNAs. Still further, the disclosure provides genome editing methods based on prime editing that involve the use of the herein disclosed prime editing fusion protein complexed with the engineered pegRNAs to install desired nucleotide sequence changes at desired sites in a genome characterized by an editing efficiency that is higher than prime editing that uses canonical pegRNAs (i.e., those pegRNAs not modified in the manner described herein). The disclosure also provides cells and kits comprising the herein disclosed modified pegRNAs, or prime editing complexes comprising said modified pegRNAs. The present disclosure also provides methods of making the disclosed modified pegRNAs comprising coupling one or more structural nucleotide motifs (e.g., an evopreQ₁-1, evopreQ₁-1, or a modified MMLV tRNA) to the terminus of the extension arm of a pegRNA, optionally through a nucleotide linker. The disclosure further provides methods for delivery of the modified pegRNAs and prime editor components to target cells for conducting genome editing at a desired edit site, as well as, methods for treating genetic disorders using prime editing in combination with the herein disclosed modified pegRNAs.

### [1] Prime Editing

The present invention relates to an improved version of “prime editing” that utilizes modified or equivalently, engineered pegRNAs which are engineered to comprise one or more structural modifications that improve one or more characteristics, including their stability, cellular lifespan, affinity for Cas9 (or more broadly, to a napDNAbp), or interaction with a target DNA (e.g., improved interaction between the primer binding site and the target DNA) thereby increasing the editing efficiency of prime editing. The inventors developed prime editing as a “search and replace” genome editing tool, which is further described in Anzalone et al., “Search-and-replace genome editing without double-strand breaks or donor DNA,” Nature, Oct. 21, 2019, 576, pp. 149-157, the contents of which are incorporated herein by reference in their entirety.

Prime editing is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) (or as in the instant disclosure, programmed with an engineered pegRNA) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution, deletion, or insertion) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand.

In various embodiments, prime editing operates by contacting a target DNA molecule (for which a change in the nucleotide sequence is desired to be introduced) with a nucleic acid programmable DNA binding protein (napDNAbp) complexed with a pegRNA (or an engineered epegRNA as in the instant disclosure). In reference to FIG. 1G, the pegRNA (or epegRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/pegRNA complex (or napDNAbp/epegRNA complex as in the instant disclosure) contacts the DNA molecule and the e/pegRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the spacer sequence of the pegRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence.” In step (d), a reverse transcriptase is introduced (as a fusion protein with the napDNAbp or in trans) which synthesizes a single strand of DNA from the 3′ end of the primed site towards the 5′ end of the e/pegRNA. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and e/pegRNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap (e.g., by FEN1 or similar enzyme that is provided in trans, as a fusion with the prime editor, or endogenously provided) that forms once the 3′ single strand DNA flap invades and hybridizes to the endogenous DNA sequence. Without being bound by theory, the cell's endogenous DNA repair and replication processes resolves the mismatched DNA to incorporate the nucleotide change(s) to form the desired altered product. The process can also be driven towards product formation with “second strand nicking,” as exemplified in FIG. 1G, or “temporal second strand nicking,” as exemplified in FIG. 1I and discussed herein.

In another embodiment of prime editing, FIG. 3F depicts the interaction of a typical pegRNA (which may be substituted with a epegRNA disclosed herein) with a target site of a double stranded DNA and the concomitant production of a 3′ single stranded DNA flap containing the genetic change of interest. The double strand DNA is shown with the top strand in the 3′ to 5′ orientation and the lower strand in the 5′ to 3′ direction. The top strand comprises the “protospacer” and the PAM sequence and is referred to as the “target strand.” The complementary lower strand is referred to as the “non-target strand.” Although not shown, the pegRNA depicted would be complexed with a Cas9 or equivalent. As shown in the schematic, the spacer sequence of the pegRNA anneals to a complementary region on the target strand, which is referred to as the protospacer, which is located just downstream of the PAM sequence and is approximately 20 nucleotides in length. This interaction forms a DNA/RNA hybrid between the spacer RNA and the protospacer DNA, and induces the formation of an R loop in the region opposite the protospacer. As taught elsewhere herein, the Cas9 protein (not shown) then induces a nick in the non-target strand, as shown. This then leads to the formation of the 3′ ssDNA flap region which, in accordance with *z*, interacts with the 3′ end of the pegRNA at the primer binding site. The 3′ end of the ssDNA flap (i.e., the reverse transcriptase primer sequence) anneals to the primer binding site (A) on the pegRNA, thereby priming reverse transcriptase. Next, reverse transcriptase (e.g., provided in trans or provided cis as a fusion protein, attached to the Cas9 construct) then polymerizes a single strand of DNA which is coded for by the edit template (B) and homology arm (C) (together which constitute the DNA synthesis template). The polymerization continues towards the 5′ end of the extension arm. The polymerized strand of ssDNA forms a ssDNA 3′ end flap which, as described elsewhere (e.g., as shown in FIG. 1G), invades the endogenous DNA, displacing the corresponding endogenous strand (which is removed as a 5′ DNA flap of endogenous DNA), and installing the desired nucleotide edit (single nucleotide base pair change, deletions, insertions (including whole genes) through DNA repair/replication rounds.

In various embodiments, prime editors rely on the mechanism of prime editing (e.g., as depicted in various embodiments of FIGS. 1A-1F). In various embodiments, prime editors comprise Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. The prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, where the specification mentions “reverse transcriptases,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., pegRNA) containing a spacer sequence that anneals to a complementary protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase.

The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random.

Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In each of these embodiments of prime editing, the modified or engineered pegRNAs described herein can be used in place of the canonical pegRNAs to increase the editing efficiency of prime editing. Without being bound by theory, the increased editing efficiency is believed to be derived from any one or more of improved pegRNA stability, improved cellular lifespan of pegRNAs, increased binding affinity of Cas9 to pegRNA, or reduced binding interaction between the primer binding site and the spacer of the epegRNA (and consequently a better interaction between the primer binding site and the target DNA).

This Detailed Description now describes the various components of prime editors contemplated herein and which may be used along with the modified or engineered pegRNAs described herein to increase the editing efficiency of prime editing.

[2] napDNAbp

The prime editors described herein may comprise a nucleic acid programmable DNA binding protein (napDNAbp).

In one aspect, a napDNAbp can be associated with or complexed with at least one guide nucleic acid (e.g., guide RNA or a pegRNA), which localizes the napDNAbp to a DNA sequence that comprises a DNA strand (i.e., a target strand) that is complementary to the guide nucleic acid, or a portion thereof (e.g., the spacer of a guide RNA which anneals to the protospacer of the DNA target). In other words, the guide nucleic-acid “programs” the napDNAbp (e.g., Cas9 or equivalent) to localize and bind to complementary sequence of the protospacer in the DNA.

Any suitable napDNAbp may be used in the prime editors described herein. In various embodiments, the napDNAbp may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new. The skilled person will be able to identify the specific CRISPR-Cas enzyme being referenced in this Application based on the nomenclature that is used, whether it is old (i.e., “legacy”) or new nomenclature. CRISPR-Cas nomenclature is extensively discussed in Makarova et al., “Classification and Nomenclature of CRISPR-Cas Systems: Where from Here?,” The CRISPR Journal, Vol. 1. No. 5, 2018, the entire contents of which are incorporated herein by reference. The particular CRISPR-Cas nomenclature used in any given instance in this Application is not limiting in any way and the skilled person will be able to identify which CRISPR-Cas enzyme is being referenced.

For example, the following type II, type V, and type VI Class 2 CRISPR-Cas enzymes have the following art-recognized old (i.e., legacy) and new names. Each of these enzymes, and/or variants thereof, may be used with the prime editors described herein:

| | |
|---|---|
| Legacy nomenclature | Current nomenclature* |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

*See Makarova et al., The CRISPR Journal, Vol. 1, No. 5, 2018

Without being bound by theory, the mechanism of action of certain napDNAbp contemplated herein includes the step of forming an R-loop whereby the napDNAbp induces the unwinding of a double-strand DNA target, thereby separating the strands in the region bound by the napDNAbp. The guide RNA spacer then hybridizes to the “target strand” at a region that is complementary to the protospacer sequence. This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the presently disclosed prime editors is not meant to be limiting in any way. The prime editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

The prime editors described herein may also comprise Cas9 equivalents, including Cas12a (Cpf1) and Cas12b1 proteins which are the result of convergent evolution. The napDNAbps used herein (e.g., SpCas9, Cas9 variant, or Cas9 equivalents) may also may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence or a reference Cas9 equivalent (e.g., Cas12a (Cpf1)).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12b1 (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the prime editors (PE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The prime editors of the present disclosure may use any suitable napDNAbp, including any suitable Cas9 or Cas9 equivalent.

### A. Wild Type Canonical SpCas9

In one embodiment, the primer editor constructs described herein may comprise the “canonical SpCas9” nuclease from S. pyogenes, which has been widely used as a tool for genome engineering and is categorized as the type II subgroup of enzymes of the Class 2 CRISPR-Cas systems. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas9 | MDKKYSIGLDIGTNSVGWAVITDEYKVPSKKFKVLGN | SEQ ID NO: |
| Streptococcus | TDRHSIKKNLIGALLFDSGETAEATRLKRTARRRYTRR | 37 |
| pyogenes | KNRICYLQEIFSNEMAKVDDSFFHRLEESFLVEEDKKH | |
| M1 | ERHPIFGNIVDEVAYHEKYPTIYHLRKKLVDSTDKADL | |
| SwissProt | RLIYLALAHMIKFRGHFLIEGDLNPDNSDVDKLFIQLVQ | |
| Accession | TYNQLFEENPINASGVDAKAILSARLSKSRRLENLIAQL | |
| No. | PGEKKNGLFGNLIALSLGLTPNFKSNFDLAEDAKLQLS | |
| Q99ZW2 | KDTYDDDLDNLLAQIGDQYADLFLAAKNLSDAILLSDI | |
| Wild type | LRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGN | |
| | SRFAWMTRKSEETITPWNFEEVVDKGASAQSFIERMTN | |
| | FDKNLPNEKVLPKHSLLYEYFTVYNELTKVKYVTEGM | |
| | RKPAFLSGEQKKAIVDLLFKTNRKVTVKQLKEDYFKKI | |
| | ECFDSVEISGVEDRFNASLGTYHDLLKIIKDKDFLDNEE | |
| | NEDILEDIVLTLTLFEDREMIEERLKTYAHLFDDKVMK | |
| | QLKRRRYTGWGRLSRKLINGIRDKQSGKTILDFLKSDG | |
| | FANRNFMQLIHDDSLTFKEDIQKAQVSGQGDSLHEHIA | |
| | NLAGSPAIKKGILQTVKVVDELVKVMGRHKPENIVIEM | |
| | ARENQTTQKGQKNSRERMKRIEEGIKELGSQILKEHPV | |
| | ENTQLQNEKLYLYYLQNGRDMYVDQELDINRLSDYD | |
| | VDHIVPQSFLKDDSIDNKVLTRSDKNRGKSDNVPSEEV | |
| | VKKMKNYWRQLLNAKLITQRKFDNLTKAERGGLSEL | |
| | DKAGFIKRQLVETRQITKHVAQILDSRMNTKYDENDK | |
| | LIREVKVITLKSKLVSDFRKDFQFYKVREINNYHHAHD | |
| | AYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEV | |
| | QTGGFSKESILPKRNSDKLIARKKDWDPKKYGGFDSPT | |
| | VAYSVLVVAKVEKGKSKKLKSVKELLGITIMERSSFEK | |
| | NPIDFLEAKGYKEVKKDLIIKLPKYSLFELENGRKRML | |
| | ASAGELQKGNELALPSKYVNFLYLASHYEKLKGSPED | |
| | NEQKQLFVEQHKHYLDEIIEQISEFSKRVILADANLDKV | |
| | LSAYNKHRDKPIREQAENIIHLFTLTNLGAPAAFKYFDT | |
| | TIDRKRYTSTKEVLDATLIHQSITGLYETRIDLSQLGGD | |
| SpCas9 | ATGGATAAAAAATATAGCATTGGCCTGGATATTGGC | SEQ ID NO: |
| Reverse | ACCAACAGCGTGGGCTGGGCGGTGATTACCGATGAA | 38 |
| translation | TATAAAGTGCCGAGCAAAAAATTTAAAGTGCTGGGC | |
| of | AACACCGATCGCCATAGCATTAAAAAAAACCTGATT | |
| SwissProt | GGCGCGCTGCTGTTTGATAGCGGCGAAACCGCGGAA | |
| Accession | GCGACCCGCCTGAAACGCACCGCGCGCCGCCGCTAT | |
| No. | ACCCGCCGCAAAAACCGCATTTGCTATCTGCAGGAA | |
| Q99ZW2 | ATTTTTAGCAACGAAATGGCGAAAGTGGATGATAGC | |
| Streptococcus | TTTTTTCATCGCCTGGAAGAAAGCTTTCTGGTGGAAG | |
| pyogenes | AAGATAAAAAACATGAACGCCATCCGATTTTTGGCA | |
| | ACATTGTGGATGAAGTGGCGTATCATGAAAAATATC | |
| | CGACCATTTATCATCTGCGCAAAAAACTGGTGGATA | |
| | GCACCGATAAAGCGGATCTGCGCCTGATTTATCTGG | |
| | CGCTGGCGCATATGATTAAATTTCGCGGCCATTTTCT | |
| | GATTGAAGGCGATCTGAACCCGGATAACAGCGATGT | |
| | GGATAAACTGTTTATTCAGCTGGTGCAGACCTATAA | |
| | CCAGCTGTTTGAAGAAAACCCGATTAACGCGAGCGG | |
| | CGTGGATGCGAAAGCGATTCTGAGCGCGCGCCTGAG | |
| | CAAAAGCCGCCGCCTGGAAAACCTGATTGCGCAGCT | |
| | GCCGGGCGAAAAAAAAAACGGCCTGTTTGGCAACCT | |
| | GATTGCGCTGAGCCTGGGCCTGACCCCGAACTTTAA | |
| | AAGCAACTTTGATCTGGCGGAAGATGCGAAACTGCA | |
| | GCTGAGCAAAGATACCTATGATGATGATCTGGATAA | |
| | CCTGCTGGCGCAGATTGGCGATCAGTATGCGGATCT | |
| | GTTTCTGGCGGCGAAAAACCTGAGCGATGCGATTCT | |
| | GCTGAGCGATATTCTGCGCGTGAACACCGAAATTAC | |
| | CAAAGCGCCGCTGAGCGCGAGCATGATTAAACGCTA | |
| | TGATGAACATCATCAGGATCTGACCCTGCTGAAAGC | |
| | GCTGGTGCGCCAGCAGCTGCCGGAAAAATATAAAG | |
| | AAATTTTTTTTGATCAGAGCAAAAACGGCTATGCGG | |
| | GCTATATTGATGGCGGCGCGAGCCAGGAAGAATTTT | |
| | ATAAATTTATTAAACCGATTCTGGAAAAAATGGATG | |
| | GCACCGAAGAACTGCTGGTGAAACTGAACCGCGAA | |
| | GATCTGCTGCGCAAACAGCGCACCTTTGATAACGGC | |
| | AGCATTCCGCATCAGATTCATCTGGGCGAACTGCAT | |
| | GCGATTCTGCGCCGCCAGGAAGATTTTTATCCGTTTC | |
| | TGAAAGATAACCGCGAAAAAATTGAAAAAATTCTG | |
| | ACCTTTCGCATTCCGTATTATGTGGGCCCGCTGGCGC | |
| | GCGGCAACAGCCGCTTTGCGTGGATGACCCGCAAAA | |
| | GCGAAGAAACCATTACCCCGTGGAACTTTGAAGAAG | |
| | TGGTGGATAAAGGCGCGAGCGCGCAGAGCTTTATTG | |
| | AACGCATGACCAACTTTGATAAAAACCTGCCGAACG | |
| | AAAAAGTGCTGCCGAAACATAGCCTGCTGTATGAAT | |
| | ATTTTACCGTGTATAACGAACTGACCAAAGTGAAAT | |
| | ATGTGACCGAAGGCATGCGCAAACCGGCGTTTCTGA | |
| | GCGGCGAACAGAAAAAAGCGATTGTGGATCTGCTGT | |
| | TTAAAACCAACCGCAAAGTGACCGTGAAACAGCTGA | |
| | AAGAAGATTATTTTAAAAAAATTGAATGCTTTGATA | |
| | GCGTGGAAATTAGCGGCGTGGAAGATCGCTTTAACG | |
| | CGAGCCTGGGCACCTATCATGATCTGCTGAAAATTA | |
| | TTAAAGATAAAGATTTTCTGGATAACGAAGAAAACG | |
| | AAGATATTCTGGAAGATATTGTGCTGACCCTGACCC | |
| | TGTTTGAAGATCGCGAAATGATTGAAGAACGCCTGA | |
| | AAACCTATGCGCATCTGTTTGATGATAAAGTGATGA | |
| | AACAGCTGAAACGCCGCCGCTATACCGGCTGGGGCC | |
| | GCCTGAGCCGCAAACTGATTAACGGCATTCGCGATA | |
| | AACAGAGCGGCAAAACCATTCTGGATTTTCTGAAAA | |
| | GCGATGGCTTTGCGAACCGCAACTTTATGCAGCTGA | |
| | TTCATGATGATAGCCTGACCTTTAAAGAAGATATTC | |
| | AGAAAGCGCAGGTGAGCGGCCAGGGCGATAGCCTG | |
| | CATGAACATATTGCGAACCTGGCGGGCAGCCCGGCG | |
| | ATTAAAAAAGGCATTCTGCAGACCGTGAAAGTGGTG | |
| | GATGAACTGGTGAAAGTGATGGGCCGCCATAAACCG | |
| | GAAAACATTGTGATTGAAATGGCGCGCGAAAACCA | |
| | GACCACCCAGAAAGGCCAGAAAAACAGCCGCGAAC | |
| | GCATGAAACGCATTGAAGAAGGCATTAAAGAACTG | |
| | GGCAGCCAGATTCTGAAAGAACATCCGGTGGAAAA | |
| | CACCCAGCTGCAGAACGAAAAACTGTATCTGTATTA | |
| | TCTGCAGAACGGCCGCGATATGTATGTGGATCAGGA | |
| | ACTGGATATTAACCGCCTGAGCGATTATGATGTGGA | |
| | TCATATTGTGCCGCAGAGCTTTCTGAAAGATGATAG | |
| | CATTGATAACAAAGTGCTGACCCGCAGCGATAAAAA | |
| | CCGCGGCAAAAGCGATAACGTGCCGAGCGAAGAAG | |
| | TGGTGAAAAAAATGAAAAACTATTGGCGCCAGCTGC | |
| | TGAACGCGAAACTGATTACCCAGCGCAAATTTGATA | |
| | ACCTGACCAAAGCGGAACGCGGCGGCCTGAGCGAA | |
| | CTGGATAAAGCGGGCTTTATTAAACGCCAGCTGGTG | |
| | GAAACCCGCCAGATTACCAAACATGTGGCGCAGATT | |
| | CTGGATAGCCGCATGAACACCAAATATGATGAAAAC | |
| | GATAAACTGATTCGCGAAGTGAAAGTGATTACCCTG | |
| | AAAAGCAAACTGGTGAGCGATTTTCGCAAAGATTTT | |
| | CAGTTTTATAAAGTGCGCGAAATTAACAACTATCAT | |
| | CATGCGCATGATGCGTATCTGAACGCGGTGGTGGGC | |
| | ACCGCGCTGATTAAAAAATATCCGAAACTGGAAAGC | |
| | GAATTTGTGTATGGCGATTATAAAGTGTATGATGTG | |
| | CGCAAAATGATTGCGAAAAGCGAACAGGAAATTGG | |
| | CAAAGCGACCGCGAAATATTTTTTTTATAGCAACAT | |
| | TATGAACTTTTTTAAAACCGAAATTACCCTGGCGAA | |
| | CGGCGAAATTCGCAAACGCCCGCTGATTGAAACCAA | |
| | CGGCGAAACCGGCGAAATTGTGTGGGATAAAGGCC | |
| | GCGATTTTGCGACCGTGCGCAAAGTGCTGAGCATGC | |
| | CGCAGGTGAACATTGTGAAAAAAACCGAAGTGCAG | |
| | ACCGGCGGCTTTAGCAAAGAAAGCATTCTGCCGAAA | |
| | CGCAACAGCGATAAACTGATTGCGCGCAAAAAAGA | |
| | TTGGGATCCGAAAAAATATGGCGGCTTTGATAGCCC | |
| | GACCGTGGCGTATAGCGTGCTGGTGGTGGCGAAAGT | |
| | GGAAAAAGGCAAAAGCAAAAAACTGAAAAGCGTGA | |
| | AAGAACTGCTGGGCATTACCATTATGGAACGCAGCA | |
| | GCTTTGAAAAAAACCCGATTGATTTTCTGGAAGCGA | |
| | AAGGCTATAAAGAAGTGAAAAAAGATCTGATTATTA | |
| | AACTGCCGAAATATAGCCTGTTTGAACTGGAAAACG | |
| | GCCGCAAACGCATGCTGGCGAGCGCGGGCGAACTG | |
| | CAGAAAGGCAACGAACTGGCGCTGCCGAGCAAATA | |
| | TGTGAACTTTCTGTATCTGGCGAGCCATTATGAAAA | |
| | ACTGAAAGGCAGCCCGGAAGATAACGAACAGAAAC | |
| | AGCTGTTTGTGGAACAGCATAAACATTATCTGGATG | |
| | AAATTATTGAACAGATTAGCGAATTTAGCAAACGCG | |
| | TGATTCTGGCGGATGCGAACCTGGATAAAGTGCTGA | |
| | GCGCGTATAACAAACATCGCGATAAACCGATTCGCG | |
| | AACAGGCGGAAAACATTATTCATCTGTTTACCCTGA | |
| | CCAACCTGGGCGCGCCGGCGGCGTTTAAATATTTTG | |
| | ATACCACCATTGATCGCAAACGCTATACCAGCACCA | |
| | AAGAAGTGCTGGATGCGACCCTGATTCATCAGAGCA | |
| | TTACCGGCCTGTATGAAACCCGCATTGATCTGAGCC | |
| | AGCTGGGCGGCGAT | |

The prime editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 (SEQ ID NO: 37) entry, which include:

| SpCas9 mutation (relative to the amino | Function/Characteristic (as reported) (see |
|---|---|
| acid sequence of the canonical SpCas9 | UniProtKB - Q99ZW2 (CAS9_STRPT1) entry - |
| sequence, SEQ ID NO: 37) | incorporated herein by reference) |
| D10A | Nickase mutant which cleaves the protospacer |
| | strand (but no cleavage of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the non-protospacer |
| | strand but does not cleave the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCA | SEQ ID NO: |
| Streptococcus | CAAATAGCGTCGGATGGGCGGTGATCACTGATGATTA | 39 |
| pyogenes | TAAGGTTCCGTCTAAAAAGTTCAAGGTTCTGGGAAAT | |
| MGAS1882 | ACAGACCGCCACAGTATCAAAAAAAATCTTATAGGGG | |
| wild type | CTCTTTTATTTGGCAGTGGAGAGACAGCGGAAGCGAC | |
| NC_017053.1 | TCGTCTCAAACGGACAGCTCGTAGAAGGTATACACGT | |
| | CGGAAGAATCGTATTTGTTATCTACAGGAGATTTTTTC | |
| | AAATGAGATGGCGAAAGTAGATGATAGTTTCTTTCATC | |
| | GACTTGAAGAGTCTTTTTTGGTGGAAGAAGACAAGAA | |
| | GCATGAACGTCATCCTATTTTTGGAAATATAGTAGATG | |
| | AAGTTGCTTATCATGAGAAATATCCAACTATCTATCAT | |
| | CTGCGAAAAAAATTGGCAGATTCTACTGATAAAGCGG | |
| | ATTTGCGCTTAATCTATTTGGCCTTAGCGCATATGATT | |
| | AAGTTTCGTGGTCATTTTTTGATTGAGGGAGATTTAAA | |
| | TCCTGATAATAGTGATGTGGACAAACTATTTATCCAGT | |
| | TGGTACAAATCTACAATCAATTATTTGAAGAAAACCCT | |
| | ATTAACGCAAGTAGAGTAGATGCTAAAGCGATTCTTTC | |
| | TGCACGATTGAGTAAATCAAGACGATTAGAAAATCTC | |
| | ATTGCTCAGCTCCCCGGTGAGAAGAGAAATGGCTTGTT | |
| | TGGGAATCTCATTGCTTTGTCATTGGGATTGACCCCTA | |
| | ATTTTAAATCAAATTTTGATTTGGCAGAAGATGCTAAA | |
| | TTACAGCTTTCAAAAGATACTTACGATGATGATTTAGA | |
| | TAATTTATTGGCGCAAATTGGAGATCAATATGCTGATT | |
| | TGTTTTTGGCAGCTAAGAATTTATCAGATGCTATTTTA | |
| | CTTTCAGATATCCTAAGAGTAAATAGTGAAATAACTA | |
| | AGGCTCCCCTATCAGCTTCAATGATTAAGCGCTACGAT | |
| | GAACATCATCAAGACTTGACTCTTTTAAAAGCTTTAGT | |
| | TCGACAACAACTTCCAGAAAAGTATAAAGAAATCTTT | |
| | TTTGATCAATCAAAAAACGGATATGCAGGTTATATTGA | |
| | TGGGGGAGCTAGCCAAGAAGAATTTTATAAATTTATC | |
| | AAACCAATTTTAGAAAAAATGGATGGTACTGAGGAAT | |
| | TATTGGTGAAACTAAATCGTGAAGATTTGCTGCGCAA | |
| | GCAACGGACCTTTGACAACGGCTCTATTCCCCATCAAA | |
| | TTCACTTGGGTGAGCTGCATGCTATTTTGAGAAGACAA | |
| | GAAGACTTTTATCCATTTTTAAAAGACAATCGTGAGAA | |
| | GATTGAAAAAATCTTGACTTTTCGAATTCCTTATTATG | |
| | TTGGTCCATTGGCGCGTGGCAATAGTCGTTTTGCATGG | |
| | ATGACTCGGAAGTCTGAAGAAACAATTACCCCATGGA | |
| | ATTTTGAAGAAGTTGTCGATAAAGGTGCTTCAGCTCAA | |
| | TCATTTATTGAACGCATGACAAACTTTGATAAAAATCT | |
| | TCCAAATGAAAAAGTACTACCAAAACATAGTTTGCTTT | |
| | ATGAGTATTTTACGGTTTATAACGAATTGACAAAGGTC | |
| | AAATATGTTACTGAGGGAATGCGAAAACCAGCATTTC | |
| | TTTCAGGTGAACAGAAGAAAGCCATTGTTGATTTACTC | |
| | TTCAAAACAAATCGAAAAGTAACCGTTAAGCAATTAA | |
| | AAGAAGATTATTTCAAAAAAATAGAATGTTTTGATAG | |
| | TGTTGAAATTTCAGGAGTTGAAGATAGATTTAATGCTT | |
| | CATTAGGCGCCTACCATGATTTGCTAAAAATTATTAAA | |
| | GATAAAGATTTTTTGGATAATGAAGAAAATGAAGATA | |
| | TCTTAGAGGATATTGTTTTAACATTGACCTTATTTGAA | |
| | GATAGGGGGATGATTGAGGAAAGACTTAAAACATATG | |
| | CTCACCTCTTTGATGATAAGGTGATGAAACAGCTTAAA | |
| | CGTCGCCGTTATACTGGTTGGGGACGTTTGTCTCGAAA | |
| | ATTGATTAATGGTATTAGGGATAAGCAATCTGGCAAA | |
| | ACAATATTAGATTTTTTGAAATCAGATGGTTTTGCCAA | |
| | TCGCAATTTTATGCAGCTGATCCATGATGATAGTTTGA | |
| | CATTTAAAGAAGATATTCAAAAAGCACAGGTGTCTGG | |
| | ACAAGGCCATAGTTTACATGAACAGATTGCTAACTTA | |
| | GCTGGCAGTCCTGCTATTAAAAAAGGTATTTTACAGAC | |
| | TGTAAAAATTGTTGATGAACTGGTCAAAGTAATGGGG | |
| | CATAAGCCAGAAAATATCGTTATTGAAATGGCACGTG | |
| | AAAATCAGACAACTCAAAAGGGCCAGAAAAATTCGCG | |
| | AGAGCGTATGAAACGAATCGAAGAAGGTATCAAAGA | |
| | ATTAGGAAGTCAGATTCTTAAAGAGCATCCTGTTGAA | |
| | AATACTCAATTGCAAAATGAAAAGCTCTATCTCTATTA | |
| | TCTACAAAATGGAAGAGACATGTATGTGGACCAAGAA | |
| | TTAGATATTAATCGTTTAAGTGATTATGATGTCGATCA | |
| | CATTGTTCCACAAAGTTTCATTAAAGACGATTCAATAG | |
| | ACAATAAGGTACTAACGCGTTCTGATAAAAATCGTGG | |
| | TAAATCGGATAACGTTCCAAGTGAAGAAGTAGTCAAA | |
| | AAGATGAAAAACTATTGGAGACAACTTCTAAACGCCA | |
| | AGTTAATCACTCAACGTAAGTTTGATAATTTAACGAAA | |
| | GCTGAACGTGGAGGTTTGAGTGAACTTGATAAAGCTG | |
| | GTTTTATCAAACGCCAATTGGTTGAAACTCGCCAAATC | |
| | ACTAAGCATGTGGCACAAATTTTGGATAGTCGCATGA | |
| | ATACTAAATACGATGAAAATGATAAACTTATTCGAGA | |
| | GGTTAAAGTGATTACCTTAAAATCTAAATTAGTTTCTG | |
| | ACTTCCGAAAAGATTTCCAATTCTATAAAGTACGTGAG | |
| | ATTAACAATTACCATCATGCCCATGATGCGTATCTAAA | |
| | TGCCGTCGTTGGAACTGCTTTGATTAAGAAATATCCAA | |
| | AACTTGAATCGGAGTTTGTCTATGGTGATTATAAAGTT | |
| | TATGATGTTCGTAAAATGATTGCTAAGTCTGAGCAAGA | |
| | AATAGGCAAAGCAACCGCAAAATATTTCTTTTACTCTA | |
| | ATATCATGAACTTCTTCAAAACAGAAATTACACTTGCA | |
| | AATGGAGAGATTCGCAAACGCCCTCTAATCGAAACTA | |
| | ATGGGGAAACTGGAGAAATTGTCTGGGATAAAGGGCG | |
| | AGATTTTGCCACAGTGCGCAAAGTATTGTCCATGCCCC | |
| | AAGTCAATATTGTCAAGAAAACAGAAGTACAGACAGG | |
| | CGGATTCTCCAAGGAGTCAATTTTACCAAAAAGAAAT | |
| | TCGGACAAGCTTATTGCTCGTAAAAAAGACTGGGATC | |
| | CAAAAAAATATGGTGGTTTTGATAGTCCAACGGTAGC | |
| | TTATTCAGTCCTAGTGGTTGCTAAGGTGGAAAAAGGG | |
| | AAATCGAAGAAGTTAAAATCCGTTAAAGAGTTACTAG | |
| | GGATCACAATTATGGAAAGAAGTTCCTTTGAAAAAAA | |
| | TCCGATTGACTTTTTAGAAGCTAAAGGATATAAGGAA | |
| | GTTAAAAAAGACTTAATCATTAAACTACCTAAATATA | |
| | GTCTTTTTGAGTTAGAAAACGGTCGTAAACGGATGCTG | |
| | GCTAGTGCCGGAGAATTACAAAAAGGAAATGAGCTGG | |
| | CTCTGCCAAGCAAATATGTGAATTTTTTATATTTAGCT | |
| | AGTCATTATGAAAAGTTGAAGGGTAGTCCAGAAGATA | |
| | ACGAACAAAAACAATTGTTTGTGGAGCAGCATAAGCA | |
| | TTATTTAGATGAGATTATTGAGCAAATCAGTGAATTTT | |
| | CTAAGCGTGTTATTTTAGCAGATGCCAATTTAGATAAA | |
| | GTTCTTAGTGCATATAACAAACATAGAGACAAACCAA | |
| | TACGTGAACAAGCAGAAAATATTATTCATTTATTTACG | |
| | TTGACGAATCTTGGAGCTCCCGCTGCTTTTAAATATTT | |
| | TGATACAACAATTGATCGTAAACGATATACGTCTACA | |
| | AAAGAAGTTTTAGATGCCACTCTTATCCATCAATCCAT | |
| | CACTGGTCTTTATGAAACACGCATTGATTTGAGTCAGC | |
| | TAGGAGGTGACTGA | |
| SpCas9 | MDKKYSIGLDIGTNSVGWAVITDDYKVPSKKFKVLGNT | SEQ ID NO: |
| Streptococcus | DRHSIKKNLIGALLFGSGETAEATRLKRTARRRYTRRKN | 40 |
| pyogenes | RICYLQEIFSNEMAKVDDSFFHRLEESFLVEEDKKHERHP | |
| MGAS1882 | IFGNIVDEVAYHEKYPTIYHLRKKLADSTDKADLRLIYLA | |
| wild type | LAHMIKFRGHFLIEGDLNPDNSDVDKLFIQLVQIYNQLFE | |
| NC_017053.1 | ENPINASRVDAKAILSARLSKSRRLENLIAQLPGEKRNGL | |
| | FGNLIALSLGLTPNFKSNFDLAEDAKLQLSKDTYDDDLD | |
| | NLLAQIGDQYADLFLAAKNLSDAILLSDILRVNSEITKAP | |
| | LSASMIKRYDEHHQDLTLLKALVRQQLPEKYKEIFFDQS | |
| | KNGYAGYIDGGASQEEFYKFIKPILEKMDGTEELLVKLN | |
| | REDLLRKQRTFDNGSIPHQIHLGELHAILRRQEDFYPFLK | |
| | DNREKIEKILTFRIPYYVGPLARGNSRFAWMTRKSEETITP | |
| | WNFEEVVDKGASAQSFIERMTNFDKNLPNEKVLPKHSLL | |
| | YEYFTVYNELTKVKYVTEGMRKPAFLSGEQKKAIVDLLF | |
| | KTNRKVTVKQLKEDYFKKIECFDSVEISGVEDRFNASLG | |
| | AYHDLLKIIKDKDFLDNEENEDILEDIVLTLTLFEDRGMIE | |
| | ERLKTYAHLFDDKVMKQLKRRRYTGWGRLSRKLINGIR | |
| | DKQSGKTILDFLKSDGFANRNFMQLIHDDSLTFKEDIQKA | |
| | QVSGQGHSLHEQIANLAGSPAIKKGILQTVKIVDELVKV | |
| | MGHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFIKDDSIDNKVLTRSDKNRGKSD | |
| | NVPSEEVVKKMKNYWRQLLNAKLITQRKFDNLTKAERG | |
| | GLSELDKAGFIKRQLVETRQITKHVAQILDSRMNTKYDE | |
| | NDKLIREVKVITLKSKLVSDFRKDFQFYKVREINNYHHA | |
| | HDAYLNAVVGTALIKKYPKLESEFVYGDYKVYDVRKMI | |
| | AKSEQEIGKATAKYFFYSNIMNFFKTEITLANGEIRKRPLI | |
| | ETNGETGEIVWDKGRDFATVRKVLSMPQVNIVKKTEVQ | |
| | TGGFSKESILPKRNSDKLIARKKDWDPKKYGGFDSPTVA | |
| | YSVLVVAKVEKGKSKKLKSVKELLGITIMERSSFEKNPID | |
| | FLEAKGYKEVKKDLIIKLPKYSLFELENGRKRMLASAGE | |
| | LQKGNELALPSKYVNFLYLASHYEKLKGSPEDNEQKQLF | |
| | VEQHKHYLDEIIEQISEFSKRVILADANLDKVLSAYNKHR | |
| | DKPIREQAENIIHLFTLTNLGAPAAFKYFDTTIDRKRYTST | |
| | KEVLDATLIHQSITGLYETRIDLSQLGGD | |
| SpCas9 | ATGGATAAAAAGTATTCTATTGGTTTAGACATCGGCAC | SEQ ID NO: |
| Streptococcus | TAATTCCGTTGGATGGGCTGTCATAACCGATGAATACA | 41 |
| pyogenes | AAGTACCTTCAAAGAAATTTAAGGTGTTGGGGAACAC | |
| wild type | AGACCGTCATTCGATTAAAAAGAATCTTATCGGTGCCC | |
| SWBC2D7W014 | TCCTATTCGATAGTGGCGAAACGGCAGAGGCGACTCG | |
| | CCTGAAACGAACCGCTCGGAGAAGGTATACACGTCGC | |
| | AAGAACCGAATATGTTACTTACAAGAAATTTTTAGCA | |
| | ATGAGATGGCCAAAGTTGACGATTCTTTCTTTCACCGT | |
| | TTGGAAGAGTCCTTCCTTGTCGAAGAGGACAAGAAAC | |
| | ATGAACGGCACCCCATCTTTGGAAACATAGTAGATGA | |
| | GGTGGCATATCATGAAAAGTACCCAACGATTTATCAC | |
| | CTCAGAAAAAAGCTAGTTGACTCAACTGATAAAGCGG | |
| | ACCTGAGGTTAATCTACTTGGCTCTTGCCCATATGATA | |
| | AAGTTCCGTGGGCACTTTCTCATTGAGGGTGATCTAAA | |
| | TCCGGACAACTCGGATGTCGACAAACTGTTCATCCAGT | |
| | TAGTACAAACCTATAATCAGTTGTTTGAAGAGAACCCT | |
| | ATAAATGCAAGTGGCGTGGATGCGAAGGCTATTCTTA | |
| | GCGCCCGCCTCTCTAAATCCCGACGGCTAGAAAACCT | |
| | GATCGCACAATTACCCGGAGAGAAGAAAAATGGGTTG | |
| | TTCGGTAACCTTATAGCGCTCTCACTAGGCCTGACACC | |
| | AAATTTTAAGTCGAACTTCGACTTAGCTGAAGATGCCA | |
| | AATTGCAGCTTAGTAAGGACACGTACGATGACGATCT | |
| | CGACAATCTACTGGCACAAATTGGAGATCAGTATGCG | |
| | GACTTATTTTTGGCTGCCAAAAACCTTAGCGATGCAAT | |
| | CCTCCTATCTGACATACTGAGAGTTAATACTGAGATTA | |
| | CCAAGGCGCCGTTATCCGCTTCAATGATCAAAAGGTA | |
| | CGATGAACATCACCAAGACTTGACACTTCTCAAGGCC | |
| | CTAGTCCGTCAGCAACTGCCTGAGAAATATAAGGAAA | |
| | TATTCTTTGATCAGTCGAAAAACGGGTACGCAGGTTAT | |
| | ATTGACGGCGGAGCGAGTCAAGAGGAATTCTACAAGT | |
| | TTATCAAACCCATATTAGAGAAGATGGATGGGACGGA | |
| | AGAGTTGCTTGTAAAACTCAATCGCGAAGATCTACTGC | |
| | GAAAGCAGCGGACTTTCGACAACGGTAGCATTCCACA | |
| | TCAAATCCACTTAGGCGAATTGCATGCTATACTTAGAA | |
| | GGCAGGAGGATTTTTATCCGTTCCTCAAAGACAATCGT | |
| | GAAAAGATTGAGAAAATCCTAACCTTTCGCATACCTTA | |
| | CTATGTGGGACCCCTGGCCCGAGGGAACTCTCGGTTCG | |
| | CATGGATGACAAGAAAGTCCGAAGAAACGATTACTCC | |
| | ATGGAATTTTGAGGAAGTTGTCGATAAAGGTGCGTCA | |
| | GCTCAATCGTTCATCGAGAGGATGACCAACTTTGACA | |
| | AGAATTTACCGAACGAAAAAGTATTGCCTAAGCACAG | |
| | TTTACTTTACGAGTATTTCACAGTGTACAATGAACTCA | |
| | CGAAAGTTAAGTATGTCACTGAGGGCATGCGTAAACC | |
| | CGCCTTTCTAAGCGGAGAACAGAAGAAAGCAATAGTA | |
| | GATCTGTTATTCAAGACCAACCGCAAAGTGACAGTTA | |
| | AGCAATTGAAAGAGGACTACTTTAAGAAAATTGAATG | |
| | CTTCGATTCTGTCGAGATCTCCGGGGTAGAAGATCGAT | |
| | TTAATGCGTCACTTGGTACGTATCATGACCTCCTAAAG | |
| | ATAATTAAAGATAAGGACTTCCTGGATAACGAAGAGA | |
| | ATGAAGATATCTTAGAAGATATAGTGTTGACTCTTACC | |
| | CTCTTTGAAGATCGGGAAATGATTGAGGAAAGACTAA | |
| | AAACATACGCTCACCTGTTCGACGATAAGGTTATGAA | |
| | ACAGTTAAAGAGGCGTCGCTATACGGGCTGGGGACGA | |
| | TTGTCGCGGAAACTTATCAACGGGATAAGAGACAAGC | |
| | AAAGTGGTAAAACTATTCTCGATTTTCTAAAGAGCGAC | |
| | GGCTTCGCCAATAGGAACTTTATGCAGCTGATCCATGA | |
| | TGACTCTTTAACCTTCAAAGAGGATATACAAAAGGCA | |
| | CAGGTTTCCGGACAAGGGGACTCATTGCACGAACATA | |
| | TTGCGAATCTTGCTGGTTCGCCAGCCATCAAAAAGGGC | |
| | ATACTCCAGACAGTCAAAGTAGTGGATGAGCTAGTTA | |
| | AGGTCATGGGACGTCACAAACCGGAAAACATTGTAAT | |
| | CGAGATGGCACGCGAAAATCAAACGACTCAGAAGGG | |
| | GCAAAAAAACAGTCGAGAGCGGATGAAGAGAATAGA | |
| | AGAGGGTATTAAAGAACTGGGCAGCCAGATCTTAAAG | |
| | GAGCATCCTGTGGAAAATACCCAATTGCAGAACGAGA | |
| | AACTTTACCTCTATTACCTACAAAATGGAAGGGACATG | |
| | TATGTTGATCAGGAACTGGACATAAACCGTTTATCTGA | |
| | TTACGACGTCGATCACATTGTACCCCAATCCTTTTTGA | |
| | AGGACGATTCAATCGACAATAAAGTGCTTACACGCTC | |
| | GGATAAGAACCGAGGGAAAAGTGACAATGTTCCAAGC | |
| | GAGGAAGTCGTAAAGAAAATGAAGAACTATTGGCGGC | |
| | AGCTCCTAAATGCGAAACTGATAACGCAAAGAAAGTT | |
| | CGATAACTTAACTAAAGCTGAGAGGGGTGGCTTGTCT | |
| | GAACTTGACAAGGCCGGATTTATTAAACGTCAGCTCGT | |
| | GGAAACCCGCCAAATCACAAAGCATGTTGCACAGATA | |
| | CTAGATTCCCGAATGAATACGAAATACGACGAGAACG | |
| | ATAAGCTGATTCGGGAAGTCAAAGTAATCACTTTAAA | |
| | GTCAAAATTGGTGTCGGACTTCAGAAAGGATTTTCAAT | |
| | TCTATAAAGTTAGGGAGATAAATAACTACCACCATGC | |
| | GCACGACGCTTATCTTAATGCCGTCGTAGGGACCGCAC | |
| | TCATTAAGAAATACCCGAAGCTAGAAAGTGAGTTTGT | |
| | GTATGGTGATTACAAAGTTTATGACGTCCGTAAGATGA | |
| | TCGCGAAAAGCGAACAGGAGATAGGCAAGGCTACAG | |
| | CCAAATACTTCTTTTATTCTAACATTATGAATTTCTTTA | |
| | AGACGGAAATCACTCTGGCAAACGGAGAGATACGCAA | |
| | ACGACCTTTAATTGAAACCAATGGGGAGACAGGTGAA | |
| | ATCGTATGGGATAAGGGCCGGGACTTCGCGACGGTGA | |
| | GAAAAGTTTTGTCCATGCCCCAAGTCAACATAGTAAA | |
| | GAAAACTGAGGTGCAGACCGGAGGGTTTTCAAAGGAA | |
| | TCGATTCTTCCAAAAAGGAATAGTGATAAGCTCATCGC | |
| | TCGTAAAAAGGACTGGGACCCGAAAAAGTACGGTGGC | |
| | TTCGATAGCCCTACAGTTGCCTATTCTGTCCTAGTAGT | |
| | GGCAAAAGTTGAGAAGGGAAAATCCAAGAAACTGAA | |
| | GTCAGTCAAAGAATTATTGGGGATAACGATTATGGAG | |
| | CGCTCGTCTTTTGAAAAGAACCCCATCGACTTCCTTGA | |
| | GGCGAAAGGTTACAAGGAAGTAAAAAAGGATCTCATA | |
| | ATTAAACTACCAAAGTATAGTCTGTTTGAGTTAGAAAA | |
| | TGGCCGAAAACGGATGTTGGCTAGCGCCGGAGAGCTT | |
| | CAAAAGGGGAACGAACTCGCACTACCGTCTAAATACG | |
| | TGAATTTCCTGTATTTAGCGTCCCATTACGAGAAGTTG | |
| | AAAGGTTCACCTGAAGATAACGAACAGAAGCAACTTT | |
| | TTGTTGAGCAGCACAAACATTATCTCGACGAAATCATA | |
| | GAGCAAATTTCGGAATTCAGTAAGAGAGTCATCCTAG | |
| | CTGATGCCAATCTGGACAAAGTATTAAGCGCATACAA | |
| | CAAGCACAGGGATAAACCCATACGTGAGCAGGCGGAA | |
| | AATATTATCCATTTGTTTACTCTTACCAACCTCGGCGCT | |
| | CCAGCCGCATTCAAGTATTTTGACACAACGATAGATCG | |
| | CAAACGATACACTTCTACCAAGGAGGTGCTAGACGCG | |
| | ACACTGATTCACCAATCCATCACGGGATTATATGAAAC | |
| | TCGGATAGATTTGTCACAGCTTGGGGGTGACGGATCCC | |
| | CCAAGAAGAAGAGGAAAGTCTCGAGCGACTACAAAG | |
| | ACCATGACGGTGATTATAAAGATCATGACATCGATTA | |
| | CAAGGATGACGATGACAAGGCTGCAGGA | |
| SpCas9 | MDKKYSIGLDIGTNSVGWAVITDEYKVPSKKFKVLGNTD | SEQ ID NO: |
| Streptococcus | RHSIKKNLIGALLFDSGETAEATRLKRTARRRYTRRKNRI | 42 |
| pyogenes | CYLQEIFSNEMAKVDDSFFHRLEESFLVEEDKKHERHPIF | |
| wild type | GNIVDEVAYHEKYPTIYHLRKKLVDSTDKADLRLIYLAL | |
| Encoded | AHMIKFRGHFLIEGDLNPDNSDVDKLFIQLVQTYNQLFEE | |
| product of | NPINASGVDAKAILSARLSKSRRLENLIAQLPGEKKNGLF | |
| SWBC2D7W014 | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGDGSPKKKRKVS | |
| | SDYKDHDGDYKDHDIDYKDDDDKAAG | |
| SpCas9 | ATGGATAAGAAATACTCAATAGGCTTAGATATCGGCA | SEQ ID NO: |
| Streptococcus | CAAATAGCGTCGGATGGGCGGTGATCACTGATGAATA | 43 |
| pyogenes | TAAGGTTCCGTCTAAAAAGTTCAAGGTTCTGGGAAAT | |
| M1GAS | ACAGACCGCCACAGTATCAAAAAAAATCTTATAGGGG | |
| wild type | CTCTTTTATTTGACAGTGGAGAGACAGCGGAAGCGAC | |
| NC_002737.2 | TCGTCTCAAACGGACAGCTCGTAGAAGGTATACACGT | |
| | CGGAAGAATCGTATTTGTTATCTACAGGAGATTTTTTC | |
| | AAATGAGATGGCGAAAGTAGATGATAGTTTCTTTCATC | |
| | GACTTGAAGAGTCTTTTTTGGTGGAAGAAGACAAGAA | |
| | GCATGAACGTCATCCTATTTTTGGAAATATAGTAGATG | |
| | AAGTTGCTTATCATGAGAAATATCCAACTATCTATCAT | |
| | CTGCGAAAAAAATTGGTAGATTCTACTGATAAAGCGG | |
| | ATTTGCGCTTAATCTATTTGGCCTTAGCGCATATGATT | |
| | AAGTTTCGTGGTCATTTTTTGATTGAGGGAGATTTAAA | |
| | TCCTGATAATAGTGATGTGGACAAACTATTTATCCAGT | |
| | TGGTACAAACCTACAATCAATTATTTGAAGAAAACCCT | |
| | ATTAACGCAAGTGGAGTAGATGCTAAAGCGATTCTTTC | |
| | TGCACGATTGAGTAAATCAAGACGATTAGAAAATCTC | |
| | ATTGCTCAGCTCCCCGGTGAGAAGAAAAATGGCTTATT | |
| | TGGGAATCTCATTGCTTTGTCATTGGGTTTGACCCCTA | |
| | ATTTTAAATCAAATTTTGATTTGGCAGAAGATGCTAAA | |
| | TTACAGCTTTCAAAAGATACTTACGATGATGATTTAGA | |
| | TAATTTATTGGCGCAAATTGGAGATCAATATGCTGATT | |
| | TGTTTTTGGCAGCTAAGAATTTATCAGATGCTATTTTA | |
| | CTTTCAGATATCCTAAGAGTAAATACTGAAATAACTAA | |
| | GGCTCCCCTATCAGCTTCAATGATTAAACGCTACGATG | |
| | AACATCATCAAGACTTGACTCTTTTAAAAGCTTTAGTT | |
| | CGACAACAACTTCCAGAAAAGTATAAAGAAATCTTTT | |
| | TTGATCAATCAAAAAACGGATATGCAGGTTATATTGAT | |
| | GGGGGAGCTAGCCAAGAAGAATTTTATAAATTTATCA | |
| | AACCAATTTTAGAAAAAATGGATGGTACTGAGGAATT | |
| | ATTGGTGAAACTAAATCGTGAAGATTTGCTGCGCAAG | |
| | CAACGGACCTTTGACAACGGCTCTATTCCCCATCAAAT | |
| | TCACTTGGGTGAGCTGCATGCTATTTTGAGAAGACAAG | |
| | AAGACTTTTATCCATTTTTAAAAGACAATCGTGAGAAG | |
| | ATTGAAAAAATCTTGACTTTTCGAATTCCTTATTATGTT | |
| | GGTCCATTGGCGCGTGGCAATAGTCGTTTTGCATGGAT | |
| | GACTCGGAAGTCTGAAGAAACAATTACCCCATGGAAT | |
| | TTTGAAGAAGTTGTCGATAAAGGTGCTTCAGCTCAATC | |
| | ATTTATTGAACGCATGACAAACTTTGATAAAAATCTTC | |
| | CAAATGAAAAAGTACTACCAAAACATAGTTTGCTTTAT | |
| | GAGTATTTTACGGTTTATAACGAATTGACAAAGGTCAA | |
| | ATATGTTACTGAAGGAATGCGAAAACCAGCATTTCTTT | |
| | CAGGTGAACAGAAGAAAGCCATTGTTGATTTACTCTTC | |
| | AAAACAAATCGAAAAGTAACCGTTAAGCAATTAAAAG | |
| | AAGATTATTTCAAAAAAATAGAATGTTTTGATAGTGTT | |
| | GAAATTTCAGGAGTTGAAGATAGATTTAATGCTTCATT | |
| | AGGTACCTACCATGATTTGCTAAAAATTATTAAAGATA | |
| | AAGATTTTTTGGATAATGAAGAAAATGAAGATATCTT | |
| | AGAGGATATTGTTTTAACATTGACCTTATTTGAAGATA | |
| | GGGAGATGATTGAGGAAAGACTTAAAACATATGCTCA | |
| | CCTCTTTGATGATAAGGTGATGAAACAGCTTAAACGTC | |
| | GCCGTTATACTGGTTGGGGACGTTTGTCTCGAAAATTG | |
| | ATTAATGGTATTAGGGATAAGCAATCTGGCAAAACAA | |
| | TATTAGATTTTTTGAAATCAGATGGTTTTGCCAATCGC | |
| | AATTTTATGCAGCTGATCCATGATGATAGTTTGACATT | |
| | TAAAGAAGACATTCAAAAAGCACAAGTGTCTGGACAA | |
| | GGCGATAGTTTACATGAACATATTGCAAATTTAGCTGG | |
| | TAGCCCTGCTATTAAAAAAGGTATTTTACAGACTGTAA | |
| | AAGTTGTTGATGAATTGGTCAAAGTAATGGGGCGGCA | |
| | TAAGCCAGAAAATATCGTTATTGAAATGGCACGTGAA | |
| | AATCAGACAACTCAAAAGGGCCAGAAAAATTCGCGAG | |
| | AGCGTATGAAACGAATCGAAGAAGGTATCAAAGAATT | |
| | AGGAAGTCAGATTCTTAAAGAGCATCCTGTTGAAAAT | |
| | ACTCAATTGCAAAATGAAAAGCTCTATCTCTATTATCT | |
| | CCAAAATGGAAGAGACATGTATGTGGACCAAGAATTA | |
| | GATATTAATCGTTTAAGTGATTATGATGTCGATCACAT | |
| | TGTTCCACAAAGTTTCCTTAAAGACGATTCAATAGACA | |
| | ATAAGGTCTTAACGCGTTCTGATAAAAATCGTGGTAA | |
| | ATCGGATAACGTTCCAAGTGAAGAAGTAGTCAAAAAG | |
| | ATGAAAAACTATTGGAGACAACTTCTAAACGCCAAGT | |
| | TAATCACTCAACGTAAGTTTGATAATTTAACGAAAGCT | |
| | GAACGTGGAGGTTTGAGTGAACTTGATAAAGCTGGTT | |
| | TTATCAAACGCCAATTGGTTGAAACTCGCCAAATCACT | |
| | AAGCATGTGGCACAAATTTTGGATAGTCGCATGAATA | |
| | CTAAATACGATGAAAATGATAAACTTATTCGAGAGGT | |
| | TAAAGTGATTACCTTAAAATCTAAATTAGTTTCTGACT | |
| | TCCGAAAAGATTTCCAATTCTATAAAGTACGTGAGATT | |
| | AACAATTACCATCATGCCCATGATGCGTATCTAAATGC | |
| | CGTCGTTGGAACTGCTTTGATTAAGAAATATCCAAAAC | |
| | TTGAATCGGAGTTTGTCTATGGTGATTATAAAGTTTAT | |
| | GATGTTCGTAAAATGATTGCTAAGTCTGAGCAAGAAA | |
| | TAGGCAAAGCAACCGCAAAATATTTCTTTTACTCTAAT | |
| | ATCATGAACTTCTTCAAAACAGAAATTACACTTGCAAA | |
| | TGGAGAGATTCGCAAACGCCCTCTAATCGAAACTAAT | |
| | GGGGAAACTGGAGAAATTGTCTGGGATAAAGGGCGAG | |
| | ATTTTGCCACAGTGCGCAAAGTATTGTCCATGCCCCAA | |
| | GTCAATATTGTCAAGAAAACAGAAGTACAGACAGGCG | |
| | GATTCTCCAAGGAGTCAATTTTACCAAAAAGAAATTC | |
| | GGACAAGCTTATTGCTCGTAAAAAAGACTGGGATCCA | |
| | AAAAAATATGGTGGTTTTGATAGTCCAACGGTAGCTTA | |
| | TTCAGTCCTAGTGGTTGCTAAGGTGGAAAAAGGGAAA | |
| | TCGAAGAAGTTAAAATCCGTTAAAGAGTTACTAGGGA | |
| | TCACAATTATGGAAAGAAGTTCCTTTGAAAAAAATCC | |
| | GATTGACTTTTTAGAAGCTAAAGGATATAAGGAAGTT | |
| | AAAAAAGACTTAATCATTAAACTACCTAAATATAGTCT | |
| | TTTTGAGTTAGAAAACGGTCGTAAACGGATGCTGGCT | |
| | AGTGCCGGAGAATTACAAAAAGGAAATGAGCTGGCTC | |
| | TGCCAAGCAAATATGTGAATTTTTTATATTTAGCTAGT | |
| | CATTATGAAAAGTTGAAGGGTAGTCCAGAAGATAACG | |
| | AACAAAAACAATTGTTTGTGGAGCAGCATAAGCATTA | |
| | TTTAGATGAGATTATTGAGCAAATCAGTGAATTTTCTA | |
| | AGCGTGTTATTTTAGCAGATGCCAATTTAGATAAAGTT | |
| | CTTAGTGCATATAACAAACATAGAGACAAACCAATAC | |
| | GTGAACAAGCAGAAAATATTATTCATTTATTTACGTTG | |
| | ACGAATCTTGGAGCTCCCGCTGCTTTTAAATATTTTGA | |
| | TACAACAATTGATCGTAAACGATATACGTCTACAAAA | |
| | GAAGTTTTAGATGCCACTCTTATCCATCAATCCATCAC | |
| | TGGTCTTTATGAAACACGCATTGATTTGAGTCAGCTAG | |
| | GAGGTGACTGA | |
| SpCas9 | MDKKYSIGLDIGTNSVGWAVITDEYKVPSKKFKVLGNTD | SEQ ID NO: |
| Streptococcus | RHSIKKNLIGALLFDSGETAEATRLKRTARRRYTRRKNRI | 37 |
| pyogenes | CYLQEIFSNEMAKVDDSFFHRLEESFLVEEDKKHERHPIF | |
| M1GAS | GNIVDEVAYHEKYPTIYHLRKKLVDSTDKADLRLIYLAL | |
| wild type | AHMIKFRGHFLIEGDLNPDNSDVDKLFIQLVQTYNQLFEE | |
| Encoded | NPINASGVDAKAILSARLSKSRRLENLIAQLPGEKKNGLF | |
| product of | GNLIALSLGLTPNFKSNFDLAEDAKLQLSKDTYDDDLDN | |
| NC_002737.2 | LLAQIGDQYADLFLAAKNLSDAILLSDILRVNTEITKAPL | |
| (100% | SASMIKRYDEHHQDLTLLKALVRQQLPEKYKEIFFDQSKN | |
| identical to | GYAGYIDGGASQEEFYKFIKPILEKMDGTEELLVKLNRE | |
| the canonical | DLLRKQRTFDNGSIPHQIHLGELHAILRRQEDFYPFLKDN | |
| Q99ZW2 | REKIEKILTFRIPYYVGPLARGNSRFAWMTRKSEETITPW | |
| wild type) | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

The prime editors described herein may include any of the above SpCas9 sequences, or any variant thereof having at least 80%, at least 85%, at least 90%, at least 95%, or at least 99% sequence identity thereto.

### B. Wild Type Cas9 Orthologs

In other embodiments, the Cas9 protein can be a wild type Cas9 ortholog from another bacterial species different from the canonical Cas9 from S. pyogenes. For example, the following Cas9 orthologs can be used in connection with the prime editor constructs described in this specification. In addition, any variant Cas9 orthologs having at least 80%, at least 85%, at least 90%, at least 95%, or at least 99% sequence identity to any of the below orthologs may also be used with the present prime editors.

| Description | Sequence | SEQ ID NO: |
|---|---|---|
| LfCas9 | MKEYHIGLDIGTSSIGWAVTDSQFKLMRIKGKTAIGVRLFEEGK | SEQ ID NO: 44 |
| Lactobacillus | TAAERRTFRTTRRRLKRRKWRLHYLDEIFAPHLQEVDENFLRR | |
| fermentum | LKQSNIHPEDPTKNQAFIGKLLFPDLLKKNERGYPTLIKMRDEL | |
| wild type | PVEQRAHYPVMNIYKLREAMINEDRQFDLREVYLAVHHIVKY | |
| GenBank: | RGHFLNNASVDKFKVGRIDFDKSFNVLNEAYEELQNGEGSFTI | |
| SNX31424.11 | EPSKVEKIGQLLLDTKMRKLDRQKAVAKLLEVKVADKEETKR | |
| | NKQIATAMSKLVLGYKADFATVAMANGNEWKIDLSSETSEDEI | |
| | EKFREELSDAQNDILTEITSLFSQIMLNEIVPNGMSISESMMDRY | |
| | WTHERQLAEVKEYLATQPASARKEFDQVYNKYIGQAPKERGF | |
| | DLEKGLKKILSKKENWKEIDELLKAGDFLPKQRTSANGVIPHQ | |
| | MHQQELDRIIEKQAKYYPWLATENPATGERDRHQAKYELDQL | |
| | VSFRIPYYVGPLVTPEVQKATSGAKFAWAKRKEDGEITPWNL | |
| | WDKIDRAESAEAFIKRMTVKDTYLLNEDVLPANSLLYQKYNV | |
| | LNELNNVRVNGRRLSVGIKQDIYTELFKKKKTVKASDVASLV | |
| | MAKTRGVNKPSVEGLSDPKKFNSNLATYLDLKSIVGDKVDDN | |
| | RYQTDLENIIEWRSVFEDGEIFADKLTEVEWLTDEQRSALVKK | |
| | RYKGWGRLSKKLLTGIVDENGQRIIDLMWNTDQNFKEIVDQP | |
| | VFKEQIDQLNQKAITNDGMTLRERVESVLDDAYTSPQNKKAIW | |
| | QVVRVVEDIVKAVGNAPKSISIEFARNEGNKGEITRSRRTQLQK | |
| | LFEDQAHELVKDTSLTEELEKAPDLSDRYYFYFTQGGKDMYT | |
| | GDPINFDEISTKYDIDHILPQSFVKDNSLDNRVLTSRKENNKKS | |
| | DQVPAKLYAAKMKPYWNQLLKQGLITQRKFENLTKDVDQNIK | |
| | YRSLGFVKRQLVETRQVIKLTANILGSMYQEAGTEIIETRAGLT | |
| | KQLREEFDLPKVREVNDYHHAVDAYLTTFAGQYLNRRYPKLR | |
| | SFFVYGEYMKFKHGSDLKLRNFNFFHELMEGDKSQGKVVDQQ | |
| | TGELITTRDEVAKSFDRLLNMKYMLVSKEVHDRSDQLYGATIV | |
| | TAKESGKLTSPIEIKKNRLVDLYGAYTNGTSAFMTIIKFTGNKP | |
| | KYKVIGIPTTSAASLKRAGKPGSESYNQELHRIIKSNPKVKKGF | |
| | EIVVPHVSYGQLIVDGDCKFTLASPTVQHPATQLVLSKKSLETI | |
| | SSGYKILKDKPAIANERLIRVFDEVVGQMNRYFTIFDQRSNRQK | |
| | VADARDKFLSLPTESKYEGAKKVQVGKTEVITNLLMGLHANA | |
| | TQGDLKVLGLATFGFFQSTTGLSLSEDTMIVYQSPTGLFERRIC | |
| | LKDI | |
| SaCas9 | MDKKYSIGLDIGTNSVGWAVITDEYKVPSKKFKVLGNTDRHSI | SEQ ID NO: 37 |
| Staphylococcus | KKNLIGALLFDSGETAEATRLKRTARRRYTRRKNRICYLQEIFS | |
| aureus | NEMAKVDDSFFHRLEESFLVEEDKKHERHPIFGNIVDEVAYHE | |
| wild type | KYPTIYHLRKKLVDSTDKADLRLIYLALAHMIKFRGHFLIEGDL | |
| GenBank: | NPDNSDVDKLFIQLVQTYNQLFEENPINASGVDAKAILSARLSK | |
| AYD60528.1 | SRRLENLIAQLPGEKKNGLFGNLIALSLGLTPNFKSNFDLAEDA | |
| | KLQLSKDTYDDDLDNLLAQIGDQYADLFLAAKNLSDAILLSDI | |
| | LRVNTEITKAPLSASMIKRYDEHHQDLTLLKALVRQQLPEKYK | |
| | EIFFDQSKNGYAGYIDGGASQEEFYKFIKPILEKMDGTEELLVK | |
| | LNREDLLRKQRTFDNGSIPHQIHLGELHAILRRQEDFYPFLKDN | |
| | REKIEKILTFRIPYYVGPLARGNSRFAWMTRKSEETITPWNFEE | |
| | VVDKGASAQSFIERMTNFDKNLPNEKVLPKHSLLYEYFTVYNE | |
| | LTKVKYVTEGMRKPAFLSGEQKKAIVDLLFKTNRKVTVKQLK | |
| | EDYFKKIECFDSVEISGVEDRFNASLGTYHDLLKIIKDKDFLDN | |
| | EENEDILEDIVLTLTLFEDREMIEERLKTYAHLFDDKVMKQLKR | |
| | RRYTGWGRLSRKLINGIRDKQSGKTILDFLKSDGFANRNFMQLI | |
| | HDDSLTFKEDIQKAQVSGQGDSLHEHIANLAGSPAIKKGILQTV | |
| | KVVDELVKVMGRHKPENIVIEMARENQTTQKGQKNSRERMK | |
| | RIEEGIKELGSQILKEHPVENTQLQNEKLYLYYLQNGRDMYVD | |
| | QELDINRLSDYDVDHIVPQSFLKDDSIDNKVLTRSDKNRGKSD | |
| | NVPSEEVVKKMKNYWRQLLNAKLITQRKFDNLTKAERGGLSE | |
| | LDKAGFIKRQLVETRQITKHVAQILDSRMNTKYDENDKLIREV | |
| | KVITLKSKLVSDFRKDFQFYKVREINNYHHAHDAYLNAVVGT | |
| | ALIKKYPKLESEFVYGDYKVYDVRKMIAKSEQEIGKATAKYFF | |
| | YSNIMNFFKTEITLANGEIRKRPLIETNGETGEIVWDKGRDFAT | |
| | VRKVLSMPQVNIVKKTEVQTGGFSKESILPKRNSDKLIARKKD | |
| | WDPKKYGGFDSPTVAYSVLVVAKVEKGKSKKLKSVKELLGITI | |
| | MERSSFEKNPIDFLEAKGYKEVKKDLIIKLPKYSLFELENGRKR | |
| | MLASAGELQKGNELALPSKYVNFLYLASHYEKLKGSPEDNEQ | |
| | KQLFVEQHKHYLDEIIEQISEFSKRVILADANLDKVLSAYNKHR | |
| | DKPIREQAENIIHLFTLTNLGAPAAFKYFDTTIDRKRYTSTKEVL | |
| | DATLIHQSITGLYETRIDLSQLGGD | |
| SaCas9 | MGKRNYILGLDIGITSVGYGIIDYETRDVIDAGVRLFKEANVEN | SEQ ID NO: 45 |
| Staphylococcus | NEGRRSKRGARRLKRRRRHRIQRVKKLLFDYNLLTDHSELSGI | |
| aureus | NPYEARVKGLSQKLSEEEFSAALLHLAKRRGVHNVNEVEEDT | |
| | GNELSTKEQISRNSKALEEKYVAELQLERLKKDGEVRGSINRFK | |
| | TSDYVKEAKQLLKVQKAYHQLDQSFIDTYIDLLETRRTYYEGP | |
| | GEGSPFGWKDIKEWYEMLMGHCTYFPEELRSVKYAYNADLY | |
| | NALNDLNNLVITRDENEKLEYYEKFQIIENVFKQKKKPTLKQIA | |
| | KEILVNEEDIKGYRVTSTGKPEFTNLKVYHDIKDITARKEIIENA | |
| | ELLDQIAKILTIYQSSEDIQEELTNLNSELTQEEIEQISNLKGYTG | |
| | THNLSLKAINLILDELWHTNDNQIAIFNRLKLVPKKVDLSQQKE | |
| | IPTTLVDDFILSPVVKRSFIQSIKVINAIIKKYGLPNDIIIELAREKN | |
| | SKDAQKMINEMQKRNRQTNERIEEIIRTTGKENAKYLIEKIKLH | |
| | DMQEGKCLYSLEAIPLEDLLNNPFNYEVDHIIPRSVSFDNSFNN | |
| | KVLVKQEENSKKGNRTPFQYLSSSDSKISYETFKKHILNLAKGK | |
| | GRISKTKKEYLLEERDINRFSVQKDFINRNLVDTRYATRGLMN | |
| | LLRSYFRVNNLDVKVKSINGGFTSFLRRKWKFKKERNKGYKH | |
| | HAEDALIIANADFIFKEWKKLDKAKKVMENQMFEEKQAESMP | |
| | EIETEQEYKEIFITPHQIKHIKDFKDYKYSHRVDKKPNRKLINDT | |
| | LYSTRKDDKGNTLIVNNLNGLYDKDNDKLKKLINKSPEKLLM | |
| | YHHDPQTYQKLKLIMEQYGDEKNPLYKYYEETGNYLTKYSKK | |
| | DNGPVIKKIKYYGNKLNAHLDITDDYPNSRNKVVKLSLKPYRF | |
| | DVYLDNGVYKFVTVKNLDVIKKENYYEVNSKCYEEAKKLKKI | |
| | SNQAEFIASFYKNDLIKINGELYRVIGVNNDLLNRIEVNMIDITY | |
| | REYLENMNDKRPPHIIKTIASKTQSIKKYSTDILGNLYEVKSKK | |
| | HPQIIKK | |
| StCas9 | MLFNKCIIISINLDFSNKEKCMTKPYSIGLDIGTNSVGWAVITDN | SEQ ID NO: 46 |
| Streptococcus | YKVPSKKMKVLGNTSKKYIKKNLLGVLLFDSGITAEGRRLKRT | |
| thermophilus | ARRRYTRRRNRILYLQEIFSTEMATLDDAFFQRLDDSFLVPDDK | |
| UniProtKB/ | RDSKYPIFGNLVEEKVYHDEFPTIYHLRKYLADSTKKADLRLV | |
| Swiss-Prot: | YLALAHMIKYRGHFLIEGEFNSKNNDIQKNFQDFLDTYNAIFES | |
| G3ECR1.2 | DLSLENSKQLEEIVKDKISKLEKKDRILKLFPGEKNSGIFSEFLK | |
| Wild type | LIVGNQADFRKCFNLDEKASLHFSKESYDEDLETLLGYIGDDYS | |
| | DVFLKAKKLYDAILLSGFLTVTDNETEAPLSSAMIKRYNEHKE | |
| | DLALLKEYIRNISLKTYNEVFKDDTKNGYAGYIDGKTNQEDFY | |
| | VYLKNLLAEFEGADYFLEKIDREDFLRKQRTFDNGSIPYQIHLQ | |
| | EMRAILDKQAKFYPFLAKNKERIEKILTFRIPYYVGPLARGNSD | |
| | FAWSIRKRNEKITPWNFEDVIDKESSAEAFINRMTSFDLYLPEE | |
| | KVLPKHSLLYETFNVYNELTKVRFIAESMRDYQFLDSKQKKDI | |
| | VRLYFKDKRKVTDKDIIEYLHAIYGYDGIELKGIEKQFNSSLST | |
| | YHDLLNIINDKEFLDDSSNEAIIEEIIHTLTIFEDREMIKQRLSKFE | |
| | NIFDKSVLKKLSRRHYTGWGKLSAKLINGIRDEKSGNTILDYLI | |
| | DDGISNRNFMQLIHDDALSFKKKIQKAQIIGDEDKGNIKEVVKS | |
| | LPGSPAIKKGILQSIKIVDELVKVMGGRKPESIVVEMARENQYT | |
| | NQGKSNSQQRLKRLEKSLKELGSKILKENIPAKLSKIDNNALQN | |
| | DRLYLYYLQNGKDMYTGDDLDIDRLSNYDIDHIIPQAFLKDNSI | |
| | DNKVLVSSASNRGKSDDFPSLEVVKKRKTFWYQLLKSKLISQR | |
| | KFDNLTKAERGGLLPEDKAGFIQRQLVETRQITKHVARLLDEK | |
| | FNNKKDENNRAVRTVKIITLKSTLVSQFRKDFELYKVREINDFH | |
| | HAHDAYLNAVIASALLKKYPKLEPEFVYGDYPKYNSFRERKSA | |
| | TEKVYFYSNIMNIFKKSISLADGRVIERPLIEVNEETGESVWNKE | |
| | SDLATVRRVLSYPQVNVVKKVEEQNHGLDRGKPKGLFNANLS | |
| | SKPKPNSNENLVGAKEYLDPKKYGGYAGISNSFAVLVKGTIEK | |
| | GAKKKITNVLEFQGISILDRINYRKDKLNFLLEKGYKDIELIIELP | |
| | KYSLFELSDGSRRMLASILSTNNKRGEIHKGNQIFLSQKFVKLL | |
| | YHAKRISNTINENHRKYVENHKKEFEELFYYILEFNENYVGAK | |
| | KNGKLLNSAFQSWQNHSIDELCSSFIGPTGSERKGLFELTSRGS | |
| | AADFEFLGVKIPRYRDYTPSSLLKDATLIHQSVTGLYETRIDLA | |
| | KLGEG | |
| LcCas9 | MKIKNYNLALTPSTSAVGHVEVDDDLNILEPVHHQKAIGVAKF | SEQ ID NO: 47 |
| Lactobacillus | GEGETAEARRLARSARRTTKRRANRINHYFNEIMKPEIDKVDP | |
| crispatus | LMFDRIKQAGLSPLDERKEFRTVIFDRPNIASYYHNQFPTIWHL | |
| NCBI | QKYLMITDEKADIRLIYWALHSLLKHRGHFFNTTPMSQFKPGK | |
| Reference | LNLKDDMLALDDYNDLEGLSFAVANSPEIEKVIKDRSMHKKE | |
| Sequence: | KIAELKKLIVNDVPDKDLAKRNNKIITQIVNAIMGNSFHLNFIFD | |
| WP_133478044.1 | MDLDKLTSKAWSFKLDDPELDTKFDAISGSMTDNQIGIFETLQ | |
| Wild type | KIYSAISLLDILNGSSNVVDAKNALYDKHKRDLNLYFKFLNTLP | |
| | DEIAKTLKAGYTLYIGNRKKDLLAARKLLKVNVAKNFSQDDF | |
| | YKLINKELKSIDKQGLQTRFSEKVGELVAQNNFLPVQRSSDNV | |
| | FIPYQLNAITFNKILENQGKYYDFLVKPNPAKKDRKNAPYELSQ | |
| | LMQFTIPYYVGPLVTPEEQVKSGIPKTSRFAWMVRKDNGAITP | |
| | WNFYDKVDIEATADKFIKRSIAKDSYLLSELVLPKHSLLYEKYE | |
| | VFNELSNVSLDGKKLSGGVKQILFNEVFKKTNKVNTSRILKAL | |
| | AKHNIPGSKITGLSNPEEFTSSLQTYNAWKKYFPNQIDNFAYQQ | |
| | DLEKMIEWSTVFEDHKILAKKLDEIEWLDDDQKKFVANTRLR | |
| | GWGRLSKRLLTGLKDNYGKSIMQRLETTKANFQQIVYKPEFRE | |
| | QIDKISQAAAKNQSLEDILANSYTSPSNRKAIRKTMSVVDEYIK | |
| | LNHGKEPDKIFLMFQRSEQEKGKQTEARSKQLNRILSQLKADK | |
| | SANKLFSKQLADEFSNAIKKSKYKLNDKQYFYFQQLGRDALTG | |
| | EVIDYDELYKYTVLHIIPRSKLTDDSQNNKVLTKYKIVDGSVAL | |
| | KFGNSYSDALGMPIKAFWTELNRLKLIPKGKLLNLTTDFSTLN | |
| | KYQRDGYIARQLVETQQIVKLLATIMQSRFKHTKIIEVRNSQVA | |
| | NIRYQFDYFRIKNLNEYYRGFDAYLAAVVGTYLYKVYPKARR | |
| | LFVYGQYLKPKKTNQENQDMHLDSEKKSQGFNFLWNLLYGK | |
| | QDQIFVNGTDVIAFNRKDLITKMNTVYNYKSQKISLAIDYHNG | |
| | AMFKATLFPRNDRDTAKTRKLIPKKKDYDTDIYGGYTSNVDG | |
| | YMLLAEIIKRDGNKQYGFYGVPSRLVSELDTLKKTRYTEYEEK | |
| | LKEIIKPELGVDLKKIKKIKILKNKVPFNQVIIDKGSKFFITSTSY | |
| | RWNYRQLILSAESQQTLMDLVVDPDFSNHKARKDARKNADER | |
| | LIKVYEEILYQVKNYMPMFVELHRCYEKLVDAQKTFKSLKISD | |
| | KAMVLNQILILLHSNATSPVLEKLGYHTRFTLGKKHNLISENAV | |
| | LVTQSITGLKENHVSIKQML | |
| PdCas9 | MTNEKYSIGLDIGTSSIGFAVVNDNNRVIRVKGKNAIGVRLFDE | SEQ ID NO: 48 |
| Pedicoccus | GKAAADRRSFRTTRRSFRTTRRRLSRRRWRLKLLREIFDAYITP | |
| damnosus | VDEAFFIRLKESNLSPKDSKKQYSGDILFNDRSDKDFYEKYPTI | |
| NCBI | YHLRNALMTEHRKFDVREIYLAIHHIMKFRGHFLNATPANNFK | |
| Reference | VGRLNLEEKFEELNDIYQRVFPDESIEFRTDNLEQIKEVLLDNK | |
| Sequence: | RSRADRQRTLVSDIYQSSEDKDIEKRNKAVATEILKASLGNKA | |
| WP_062913273.1 | KLNVITNVEVDKEAAKEWSITFDSESIDDDLAKIEGQMTDDGH | |
| Wild type | EIIEVLRSLYSGITLSAIVPENHTLSQSMVAKYDLHKDHLKLFK | |
| | KLINGMTDTKKAKNLRAAYDGYIDGVKGKVLPQEDFYKQVQ | |
| | VNLDDSAEANEIQTYIDQDIFMPKQRTKANGSIPHQLQQQELD | |
| | QIIENQKAYYPWLAELNPNPDKKRQQLAKYKLDELVTFRVPY | |
| | YVGPMITAKDQKNQSGAEFAWMIRKEPGNITPWNFDQKVDR | |
| | MATANQFIKRMTTTDTYLLGEDVLPAQSLLYQKFEVLNELNKI | |
| | RIDHKPISIEQKQQIFNDLFKQFKNVTIKHLQDYLVSQGQYSKR | |
| | PLIEGLADEKRFNSSLSTYSDLCGIFGAKLVEENDRQEDLEKIIE | |
| | WSTIFEDKKIYRAKLNDLTWLTDDQKEKLATKRYQGWGRLSR | |
| | KLLVGLKNSEHRNIMDILWITNENFMQIQAEPDFAKLVTDANK | |
| | GMLEKTDSQDVINDLYTSPQNKKAIRQILLVVHDIQNAMHGQA | |
| | PAKIHVEFARGEERNPRRSVQRQRQVEAAYEKVSNELVSAKVR | |
| | QEFKEAINNKRDFKDRLFLYFMQGGIDIYTGKQLNIDQLSSYQI | |
| | DHILPQAFVKDDSLTNRVLTNENQVKADSVPIDIFGKKMLSVW | |
| | GRMKDQGLISKGKYRNLTMNPENISAHTENGFINRQLVETRQV | |
| | IKLAVNILADEYGDSTQIISVKADLSHQMREDFELLKNRDVND | |
| | YHHAFDAYLAAFIGNYLLKRYPKLESYFVYGDFKKFTQKETK | |
| | MRRFNFIYDLKHCDQVVNKETGEILWTKDEDIKYIRHLFAYKK | |
| | ILVSHEVREKRGALYNQTIYKAKDDKGSGQESKKLIRIKDDKE | |
| | TKIYGGYSGKSLAYMTIVQITKKNKVSYRVIGIPTLALARLNKL | |
| | ENDSTENNGELYKIIKPQFTHYKVDKKNGEIIETTDDFKIVVSK | |
| | VRFQQLIDDAGQFFMLASDTYKNNAQQLVISNNALKAINNTNI | |
| | TDCPRDDLERLDNLRLDSAFDEIVKKMDKYFSAYDANNFREKI | |
| | RNSNLIFYQLPVEDQWENNKITELGKRTVLTRILQGLHANATTT | |
| | DMSIFKIKTPFGQLRQRSGISLSENAQLIYQSPTGLFERRVQLNK | |
| | IK | |
| FnCas9 | MKKQKFSDYYLGFDIGTNSVGWCVTDLDYNVLRFNKKDMWG | SEQ ID NO: 49 |
| Fusobaterium | SRLFEEAKTAAERRVQRNSRRRLKRRKWRLNLLEEIFSNEILKI | |
| nucleatum | DSNFFRRLKESSLWLEDKSSKEKFTLFNDDNYKDYDFYKQYPT | |
| NCBI | IFHLRNELIKNPEKKDIRLVYLAIHSIFKSRGHFLFEGQNLKEIKN | |
| Reference | FETLYNNLIAFLEDNGINKIIDKNNIEKLEKIVCDSKKGLKDKEK | |
| Sequence: | EFKEIFNSDKQLVAIFKLSVGSSVSLNDLFDTDEYKKGEVEKEK | |
| WP_060798984.1 | ISFREQIYEDDKPIYYSILGEKIELLDIAKTFYDFMVLNNILADSQ | |
| | YISEAKVKLYEEHKKDLKNLKYIIRKYNKGNYDKLFKDKNEN | |
| | NYSAYIGLNKEKSKKEVIEKSRLKIDDLIKNIKGYLPKVEEIEEK | |
| | DKAIFNKILNKIELKTILPKQRISDNGTLPYQIHEAELEKILENQS | |
| | KYYDFLNYEENGIITKDKLLMTFKFRIPYYVGPLNSYHKDKGG | |
| | NSWIVRKEEGKILPWNFEQKVDIEKSAEEFIKRMTNKCTYLNG | |
| | EDVIPKDTFLYSEYVILNELNKVQVNDEFLNEENKRKIIDELFKE | |
| | NKKVSEKKFKEYLLVKQIVDGTIELKGVKDSFNSNYISYIRFKD | |
| | IFGEKLNLDIYKEISEKSILWKCLYGDDKKIFEKKIKNEYGDILT | |
| | KDEIKKINTFKFNNWGRLSEKLLTGIEFINLETGECYSSVMDAL | |
| | RRTNYNLMELLSSKFTLQESINNENKEMNEASYRDLIEESYVSP | |
| | SLKRAIFQTLKIYEEIRKITGRVPKKVFIEMARGGDESMKNKKIP | |
| | ARQEQLKKLYDSCGNDIANFSIDIKEMKNSLISYDNNSLRQKKL | |
| | YLYYLQFGKCMYTGREIDLDRLLQNNDTYDIDHIYPRSKVIKD | |
| | DSFDNLVLVLKNENAEKSNEYPVKKEIQEKMKSFWRFLKEKN | |
| | FISDEKYKRLTGKDDFELRGFMARQLVNVRQTTKEVGKILQQI | |
| | EPEIKIVYSKAEIASSFREMFDFIKVRELNDTHHAKDAYLNIVA | |
| | GNVYNTKFTEKPYRYLQEIKENYDVKKIYNYDIKNAWDKENS | |
| | LEIVKKNMEKNTVNITRFIKEKKGQLFDLNPIKKGETSNEIISIKP | |
| | KVYNGKDDKLNEKYGYYKSLNPAYFLYVEHKEKNKRIKSFER | |
| | VNLVDVNNIKDEKSLVKYLIENKKLVEPRVIKKVYKRQVILIND | |
| | YPYSIVTLDSNKLMDFENLKPLFLENKYEKILKNVIKFLEDNQG | |
| | KSEENYKFIYLKKKDRYEKNETLESVKDRYNLEFNEMYDKFLE | |
| | KLDSKDYKNYMNNKKYQELLDVKEKFIKLNLFDKAFTLKSFL | |
| | DLFNRKTMADFSKVGLTKYLGKIQKISSNVLSKNELYLLEESVT | |
| | GLFVKKIKL | |
| EcCas9 | RRKQRIQILQELLGEEVLKTDPGFFHRMKESRYVVEDKRTLDG | SEQ ID NO: 50 |
| Enterococcus | KQVELPYALFVDKDYTDKEYYKQFPTINHLIVYLMTTSDTPDIR | |
| cecorum | LVYLALHYYMKNRGNFLHSGDINNVKDINDILEQLDNVLETFL | |
| NCBI | DGWNLKLKSYVEDIKNIYNRDLGRGERKKAFVNTLGAKTKAE | |
| Reference | KAFCSLISGGSTNLAELFDDSSLKEIETPKIEFASSSLEDKIDGIQE | |
| Sequence: | ALEDRFAVIEAAKRLYDWKTLTDILGDSSSLAEARVNSYQMH | |
| WP_047338501.1 | HEQLLELKSLVKEYLDRKVFQEVFVSLNVANNYPAYIGHTKIN | |
| Wild type | GKKKELEVKRTKRNDFYSYVKKQVIEPIKKKVSDEAVLTKLSE | |
| | IESLIEVDKYLPLQVNSDNGVIPYQVKLNELTRIFDNLENRIPVL | |
| | RENRDKIIKTFKFRIPYYVGSLNGVVKNGKCTNWMVRKEEGKI | |
| | YPWNFEDKVDLEASAEQFIRRMTNKCTYLVNEDVLPKYSLLYS | |
| | KYLVLSELNNLRIDGRPLDVKIKQDIYENVFKKNRKVTLKKIK | |
| | KYLLKEGIITDDDELSGLADDVKSSLTAYRDFKEKLGHLDLSE | |
| | AQMENIILNITLFGDDKKLLKKRLAALYPFIDDKSLNRIATLNY | |
| | RDWGRLSERFLSGITSVDQETGELRTIIQCMYETQANLMQLLA | |
| | EPYHFVEAIEKENPKVDLESISYRIVNDLYVSPAVKRQIWQTLL | |
| | VIKDIKQVMKHDPERIFIEMAREKQESKKTKSRKQVLSEVYKK | |
| | AKEYEHLFEKLNSLTEEQLRSKKIYLYFTQLGKCMYSGEPIDFE | |
| | NLVSANSNYDIDHIYPQSKTIDDSFNNIVLVKKSLNAYKSNHYP | |
| | IDKNIRDNEKVKTLWNTLVSKGLITKEKYERLIRSTPFSDEELA | |
| | GFIARQLVETRQSTKAVAEILSNWFPESEIVYSKAKNVSNFRQD | |
| | FEILKVRELNDCHHAHDAYLNIVVGNAYHTKFTNSPYRFIKNK | |
| | ANQEYNLRKLLQKVNKIESNGVVAWVGQSENNPGTIATVKKV | |
| | IRRNTVLISRMVKEVDGQLFDLTLMKKGKGQVPIKSSDERLTDI | |
| | SKYGGYNKATGAYFTFVKSKKRGKVVRSFEYVPLHLSKQFEN | |
| | NNELLKEYIEKDRGLTDVEILIPKVLINSLFRYNGSLVRITGRGD | |
| | TRLLLVHEQPLYVSNSFVQQLKSVSSYKLKKSENDNAKLTKTA | |
| | TEKLSNIDELYDGLLRKLDLPIYSYWFSSIKEYLVESRTKYIKLSI | |
| | EEKALVIFEILHLFQSDAQVPNLKILGLSTKPSRIRIQKNLKDTD | |
| | KMSIIHQSPSGIFEHEIELTSL | |
| AhCas9 | MQNGFLGITVSSEQVGWAVTNPKYELERASRKDLWGVRLFDK | SEQ ID NO: 51 |
| Anaerostipes | AETAEDRRMFRTNRRLNQRKKNRIHYLRDIFHEEVNQKDPNFF | |
| hadrus | QQLDESNFCEDDRTVEFNFDTNLYKNQFPTVYHLRKYLMETK | |
| NCBI | DKPDIRLVYLAFSKFMKNRGHFLYKGNLGEVMDFENSMKGFC | |
| Reference | ESLEKFNIDFPTLSDEQVKEVRDILCDHKIAKTVKKKNIITITKV | |
| Sequence: | KSKTAKAWIGLFCGCSVPVKVLFQDIDEEIVTDPEKISFEDASY | |
| WP_044924278.1 | DDYIANIEKGVGIYYEAIVSAKMLFDWSILNEILGDHQLLSDAM | |
| Wild type | IAEYNKHHDDLKRLQKIIKGTGSRELYQDIFINDVSGNYVCYVG | |
| | HAKTMSSADQKQFYTFLKNRLKNVNGISSEDAEWIDTEIKNGT | |
| | LLPKQTKRDNSVIPHQLQLREFELILDNMQEMYPFLKENREKLL | |
| | KIFNFVIPYYVGPLKGVVRKGESTNWMVPKKDGVIHPWNFDE | |
| | MVDKEASAECFISRMTGNCSYLFNEKVLPKNSLLYETFEVLNE | |
| | LNPLKINGEPISVELKQRIYEQLFLTGKKVTKKSLTKYLIKNGY | |
| | DKDIELSGIDNEFHSNLKSHIDFEDYDNLSDEEVEQIILRITVFED | |
| | KQLLKDYLNREFVKLSEDERKQICSLSYKGWGNLSEMLLNGIT | |
| | VTDSNGVEVSVMDMLWNTNLNLMQILSKKYGYKAEIEHYNK | |
| | EHEKTIYNREDLMDYLNIPPAQRRKVNQLITIVKSLKKTYGVPN | |
| | KIFFKISREHQDDPKRTSSRKEQLKYLYKSLKSEDEKHLMKELD | |
| | ELNDHELSNDKVYLYFLQKGRCIYSGKKLNLSRLRKSNYQNDI | |
| | DYIYPLSAVNDRSMNNKVLTGIQENRADKYTYFPVDSEIQKKM | |
| | KGFWMELVLQGFMTKEKYFRLSRENDFSKSELVSFIEREISDNQ | |
| | QSGRMIASVLQYYFPESKIVFVKEKLISSFKRDFHLISSYGHNHL | |
| | QAAKDAYITIVVGNVYHTKFTMDPAIYFKNHKRKDYDLNRLF | |
| | LENISRDGQIAWESGPYGSIQTVRKEYAQNHIAVTKRVVEVKG | |
| | GLFKQMPLKKGHGEYPLKTNDPRFGNIAQYGGYTNVTGSYFV | |
| | LVESMEKGKKRISLEYVPVYLHERLEDDPGHKLLKEYLVDHR | |
| | KLNHPKILLAKVRKNSLLKIDGFYYRLNGRSGNALILTNAVELI | |
| | MDDWQTKTANKISGYMKRRAIDKKARVYQNEFHIQELEQLYD | |
| | FYLDKLKNGVYKNRKNNQAELIHNEKEQFMELKTEDQCVLLT | |
| | EIKKLFVCSPMQADLTLIGGSKHTGMIAMSSNVTKADFAVIAE | |
| | DPLGLRNKVIYSHKGEK | |
| KvCas9 | MSQNNNKIYNIGLDIGDASVGWAVVDEHYNLLKRHGKHMWG | SEQ ID NO: 52 |
| Kandleria | SRLFTQANTAVERRSSRSTRRRYNKRRERIRLLREIMEDMVLD | |
| vitulina | VDPTFFIRLANVSFLDQEDKKDYLKENYHSNYNLFIDKDFNDK | |
| NCBI | TYYDKYPTIYHLRKHLCESKEKEDPRLIYLALHHIVKYRGNFLY | |
| Reference | EGQKFSMDVSNIEDKMIDVLRQFNEINLFEYVEDRKKIDEVLN | |
| Sequence: | VLKEPLSKKHKAEKAFALFDTTKDNKAAYKELCAALAGNKFN | |
| WP_031589969.1 | VTKMLKEAELHDEDEKDISFKFSDATFDDAFVEKQPLLGDCVE | |
| Wild type | FIDLLHDIYSWVELQNILGSAHTSEPSISAAMIQRYEDHKNDLK | |
| | LLKDVIRKYLPKKYFEVFRDEKSKKNNYCNYINHPSKTPVDEF | |
| | YKYIKKLIEKIDDPDVKTILNKIELESFMLKQNSRTNGAVPYQM | |
| | QLDELNKILENQSVYYSDLKDNEDKIRSILTFRIPYYFGPLNITK | |
| | DRQFDWIIKKEGKENERILPWNANEIVDVDKTADEFIKRMRNF | |
| | CTYFPDEPVMAKNSLTVSKYEVLNEINKLRINDHLIKRDMKDK | |
| | MLHTLFMDHKSISANAMKKWLVKNQYFSNTDDIKIEGFQKEN | |
| | ACSTSLTPWIDFTKIFGKINESNYDFIEKIIYDVTVFEDKKILRRR | |
| | LKKEYDLDEEKIKKILKLKYSGWSRLSKKLLSGIKTKYKDSTRT | |
| | PETVLEVMERTNMNLMQVINDEKLGFKKTIDDANSTSVSGKFS | |
| | YAEVQELAGSPAIKRGIWQALLIVDEIKKIMKHEPAHVYIEFAR | |
| | NEDEKERKDSFVNQMLKLYKDYDFEDETEKEANKHLKGEDA | |
| | KSKIRSERLKLYYTQMGKCMYTGKSLDIDRLDTYQVDHIVPQS | |
| | LLKDDSIDNKVLVLSSENQRKLDDLVIPSSIRNKMYGFWEKLF | |
| | NNKIISPKKFYSLIKTEFNEKDQERFINRQIVETRQITKHVAQIID | |
| | NHYENTKVVTVRADLSHQFRERYHIYKNRDINDFHHAHDAYI | |
| | ATILGTYIGHRFESLDAKYIYGEYKRIFRNQKNKGKEMKKNND | |
| | GFILNSMRNIYADKDTGEIVWDPNYIDRIKKCFYYKDCFVTKK | |
| | LEENNGTFFNVTVLPNDTNSDKDNTLATVPVNKYRSNVNKYG | |
| | GFSGVNSFIVAIKGKKKKGKKVIEVNKLTGIPLMYKNADEEIKI | |
| | NYLKQAEDLEEVQIGKEILKNQLIEKDGGLYYIVAPTEIINAKQ | |
| | LILNESQTKLVCEIYKAMKYKNYDNLDSEKIIDLYRLLINKMEL | |
| | YYPEYRKQLVKKFEDRYEQLKVISIEEKCNIIKQILATLHCNSSI | |
| | GKIMYSDFKISTTIGRLNGRTISLDDISFIAESPTGMYSKKYKL | |
| EfCas9 | MRLFEEGHTAEDRRLKRTARRRISRRRNRLRYLQAFFEEAMTD | SEQ ID NO: 53 |
| Enterococcus | LDENFFARLQESFLVPEDKKWHRHPIFAKLEDEVAYHETYPTIY | |
| faecalis | HLRKKLADSSEQADLRLIYLALAHIVKYRGHFLIEGKLSTENTS | |
| NCBI | VKDQFQQFMVIYNQTFVNGESRLVSAPLPESVLIEEELTEKASR | |
| Reference | TKKSEKVLQQFPQEKANGLFGQFLKLMVGNKADFKKVFGLEE | |
| Sequence: | EAKITYASESYEEDLEGILAKVGDEYSDVFLAAKNVYDAVELS | |
| WP_016631044.1 | TILADSDKKSHAKLSSSMIVRFTEHQEDLKKFKRFIRENCPDEY | |
| Wild type | DNLFKNEQKDGYAGYIAHAGKVSQLKFYQYVKKIIQDIAGAE | |
| | YFLEKIAQENFLRKQRTFDNGVIPHQIHLAELQAIIHRQAAYYPF | |
| | LKENQEKIEQLVTFRIPYYVGPLSKGDASTFAWLKRQSEEPIRP | |
| | WNLQETVDLDQSATAFIERMTNFDTYLPSEKVLPKHSLLYEKF | |
| | MVFNELTKISYTDDRGIKANFSGKEKEKIFDYLFKTRRKVKKK | |
| | DIIQFYRNEYNTEIVTLSGLEEDQFNASFSTYQDLLKCGLTRAEL | |
| | DHPDNAEKLEDIIKILTIFEDRQRIRTQLSTFKGQFSAEVLKKLE | |
| | RKHYTGWGRLSKKLINGIYDKESGKTILDYLVKDDGVSKHYN | |
| | RNFMQLINDSQLSFKNAIQKAQSSEHEETLSETVNELAGSPAIK | |
| | KGIYQSLKIVDELVAIMGYAPKRIVVEMARENQTTSTGKRRSIQ | |
| | RLKIVEKAMAEIGSNLLKEQPTTNEQLRDTRLFLYYMQNGKD | |
| | MYTGDELSLHRLSHYDIDHIIPQSFMKDDSLDNLVLVGSTENR | |
| | GKSDDVPSKEVVKDMKAYWEKLYAAGLISQRKFQRLTKGEQ | |
| | GGLTLEDKAHFIQRQLVETRQITKNVAGILDQRYNAKSKEKKV | |
| | QIITLKASLTSQFRSIFGLYKVREVNDYHHGQDAYLNCVVATTL | |
| | LKVYPNLAPEFVYGEYPKFQTFKENKATAKAIIYTNLLRFFTED | |
| | EPRFTKDGEILWSNSYLKTIKKELNYHQMNIVKKVEVQKGGFS | |
| | KESIKPKGPSNKLIPVKNGLDPQKYGGFDSPVVAYTVLFTHEK | |
| | GKKPLIKQEILGITIMEKTRFEQNPILFLEEKGFLRPRVLMKLPK | |
| | YTLYEFPEGRRRLLASAKEAQKGNQMVLPEHLLTLLYHAKQC | |
| | LLPNQSESLAYVEQHQPEFQEILERVVDFAEVHTLAKSKVQQIV | |
| | KLFEANQTADVKEIAASFIQLMQFNAMGAPSTFKFFQKDIERA | |
| | RYTSIKEIFDATIIYQSPTGLYETRRKVVD | |
| Staphylococcus | KRNYILGLDIGITSVGYGIIDYETRDVIDAGVRLFKEANVENNE | SEQ ID NO: 54 |
| aureus | GRRSKRGARRLKRRRRHRIQRVKKLLFDYNLLTDHSELSGINP | |
| Cas9 | YEARVKGLSQKLSEEEFSAALLHLAKRRGVHNVNEVEEDTGN | |
| | ELSTKEQISRNSKALEEKYVAELQLERLKKDGEVRGSINRFKTS | |
| | DYVKEAKQLLKVQKAYHQLDQSFIDTYIDLLETRRTYYEGPGE | |
| | GSPFGWKDIKEWYEMLMGHCTYFPEELRSVKYAYNADLYNA | |
| | LNDLNNLVITRDENEKLEYYEKFQIIENVFKQKKKPTLKQIAKE | |
| | ILVNEEDIKGYRVTSTGKPEFTNLKVYHDIKDITARKEIIENAEL | |
| | LDQIAKILTIYQSSEDIQEELTNLNSELTQEEIEQISNLKGYTGTH | |
| | NLSLKAINLILDELWHTNDNQIAIFNRLKLVPKKVDLSQQKEIP | |
| | TTLVDDFILSPVVKRSFIQSIKVINAIIKKYGLPNDIIIELAREKNS | |
| | KDAQKMINEMQKRNRQTNERIEEIIRTTGKENAKYLIEKIKLHD | |
| | MQEGKCLYSLEAIPLEDLLNNPFNYEVDHIIPRSVSFDNSFNNK | |
| | VLVKQEENSKKGNRTPFQYLSSSDSKISYETFKKHILNLAKGKG | |
| | RISKTKKEYLLEERDINRFSVQKDFINRNLVDTRYATRGLMNLL | |
| | RSYFRVNNLDVKVKSINGGFTSFLRRKWKFKKERNKGYKHHA | |
| | EDALIIANADFIFKEWKKLDKAKKVMENQMFEEKQAESMPEIE | |
| | TEQEYKEIFITPHQIKHIKDFKDYKYSHRVDKKPNRELINDTLYS | |
| | TRKDDKGNTLIVNNLNGLYDKDNDKLKKLINKSPEKLLMYHH | |
| | DPQTYQKLKLIMEQYGDEKNPLYKYYEETGNYLTKYSKKDNG | |
| | PVIKKIKYYGNKLNAHLDITDDYPNSRNKVVKLSLKPYRFDVY | |
| | LDNGVYKFVTVKNLDVIKKENYYEVNSKCYEEAKKLKKISNQ | |
| | AEFIASFYNNDLIKINGELYRVIGVNNDLLNRIEVNMIDITYREY | |
| | LENMNDKRPPRIIKTIASKTQSIKKYSTDILGNLYEVKSKKHPQII | |
| | KKG | |
| Geobacillus | MKYKIGLDIGITSIGWAVINLDIPRIEDLGVRIFDRAENPKTGESL | SEQ ID NO: 55 |
| thermodenitrificans | ALPRRLARSARRRLRRRKHRLERIRRLFVREGILTKEELNKLFE | |
| Cas9 | KKHEIDVWQLRVEALDRKLNNDELARILLHLAKRRGFRSNRKS | |
| | ERTNKENSTMLKHIEENQSILSSYRTVAEMVVKDPKFSLHKRN | |
| | KEDNYTNTVARDDLEREIKLIFAKQREYGNIVCTEAFEHEYISI | |
| | WASQRPFASKDDIEKKVGFCTFEPKEKRAPKATYTFQSFTVWE | |
| | HINKLRLVSPGGIRALTDDERRLIYKQAFHKNKITFHDVRTLLN | |
| | LPDDTRFKGLLYDRNTTLKENEKVRFLELGAYHKIRKAIDSVY | |
| | GKGAAKSFRPIDFDTFGYALTMFKDDTDIRSYLRNEYEQNGKR | |
| | MENLADKVYDEELIEELLNLSFSKFGHLSLKALRNILPYMEQGE | |
| | VYSTACERAGYTFTGPKKKQKTVLLPNIPPIANPVVMRALTQA | |
| | RKVVNAIIKKYGSPVSIHIELARELSQSFDERRKMQKEQEGNRK | |
| | KNETAIRQLVEYGLTLNPTGLDIVKFKLWSEQNGKCAYSLQPIE | |
| | IERLLEPGYTEVDHVIPYSRSLDDSYTNKVLVLTKENREKGNRT | |
| | PAEYLGLGSERWQQFETFVLTNKQFSKKKRDRLLRLHYDENEE | |
| | NEFKNRNLNDTRYISRFLANFIREHLKFADSDDKQKVYTVNGR | |
| | ITAHLRSRWNFNKNREESNLHHAVDAAIVACTTPSDIARVTAF | |
| | YQRREQNKELSKKTDPQFPQPWPHFADELQARLSKNPKESIKA | |
| | LNLGNYDNEKLESLQPVFVSRMPKRSITGAAHQETLRRYIGIDE | |
| | RSGKIQTVVKKKLSEIQLDKTGHFPMYGKESDPRTYEAIRQRLL | |
| | EHNNDPKKAFQEPLYKPKKNGELGPIIRTIKIIDTTNQVIPLNDG | |
| | KTVAYNSNIVRVDVFEKDGKYYCVPIYTIDMMKGILPNKAIEP | |
| | NKPYSEWKEMTEDYTFRFSLYPNDLIRIEFPREKTIKTAVGEEIK | |
| | IKDLFAYYQTIDSSNGGLSLVSHDNNFSLRSIGSRTLKRFEKYQ | |
| | VDVLGNIYKVRGEKRVGVASSSHSKAGETIRPL | |
| ScCas9 | MEKKYSIGLDIGTNSVGWAVITDDYKVPSKKFKVLGNTNRKSI | SEQ ID NO: 56 |
| S. canis | KKNLMGALLFDSGETAEATRLKRTARRRYTRRKNRIRYLQEIF | |
| 1375 AA | ANEMAKLDDSFFQRLEESFLVEEDKKNERHPIFGNLADEVAYH | |
| 159.2 kDa | RNYPTIYHLRKKLADSPEKADLRLIYLALAHIIKFRGHFLIEGKL | |
| | NAENSDVAKLFYQLIQTYNQLFEESPLDEIEVDAKGILSARLSK | |
| | SKRLEKLIAVFPNEKKNGLFGNIIALALGLTPNFKSNFDLTEDA | |
| | KLQLSKDTYDDDLDELLGQIGDQYADLFSAAKNLSDAILLSDIL | |
| | RSNSEVTKAPLSASMVKRYDEHHQDLALLKTLVRQQFPEKYA | |
| | EIFKDDTKNGYAGYVGIGIKHRKRTTKLATQEEFYKFIKPILEK | |
| | MDGAEELLAKLNRDDLLRKQRTFDNGSIPHQIHLKELHAILRR | |
| | QEEFYPFLKENREKIEKILTFRIPYYVGPLARGNSRFAWLTRKSE | |
| | EAITPWNFEEVVDKGASAQSFIERMTNFDEQLPNKKVLPKHSL | |
| | LYEYFTVYNELTKVKYVTERMRKPEFLSGEQKKAIVDLLFKTN | |
| | RKVTVKQLKEDYFKKIECFDSVEIIGVEDRFNASLGTYHDLLKII | |
| | KDKDFLDNEENEDILEDIVLTLTLFEDREMIEERLKTYAHLFDD | |
| | KVMKQLKRRHYTGWGRLSRKMINGIRDKQSGKTILDFLKSDG | |
| | FSNRNFMQLIHDDSLTFKEEIEKAQVSGQGDSLHEQIADLAGSP | |
| | AIKKGILQTVKIVDELVKVMGHKPENIVIEMARENQTTTKGLQ | |
| | QSRERKKRIEEGIKELESQILKENPVENTQLQNEKLYLYYLQNG | |
| | RDMYVDQELDINRLSDYDVDHIVPQSFIKDDSIDNKVLTRSVE | |
| | NRGKSDNVPSEEVVKKMKNYWRQLLNAKLITQRKFDNLTKAE | |
| | RGGLSEADKAGFIKRQLVETRQITKHVARILDSRMNTKRDKND | |
| | KPIREVKVITLKSKLVSDFRKDFQLYKVRDINNYHHAHDAYLN | |
| | AVVGTALIKKYPKLESEFVYGDYKVYDVRKMIAKSEQEIGKAT | |
| | AKRFFYSNIMNFFKTEVKLANGEIRKRPLIETNGETGEVVWNK | |
| | EKDFATVRKVLAMPQVNIVKKTEVQTGGFSKESILSKRESAKLI | |
| | PRKKGWDTRKYGGFGSPTVAYSILVVAKVEKGKAKKLKSVKV | |
| | LVGITIMEKGSYEKDPIGFLEAKGYKDIKKELIFKLPKYSLFELE | |
| | NGRRRMLASATELQKANELVLPQHLVRLLYYTQNISATTGSNN | |
| | LGYIEQHREEFKEIFEKIIDFSEKYILKNKVNSNLKSSFDEQFAVS | |
| | DSILLSNSFVSLLKYTSFGASGGFTFLDLDVKQGRLRYQTVTEV | |
| | LDATLIYQSITGLYETRTDLSQLGGD | |

The prime editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or enzymes, such as, Cas9. Cas9 homologs and/or orthologs have been described in various species, including, but not limited to, S. pyogenes and S. thermophilus. Preferably, the Cas moiety is configured (e.g., mutagenized, recombinantly engineered, or otherwise obtained from nature) as a nickase, i.e., capable of cleaving only a single strand of the target double stranded DNA. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

C. Dead napDNAbp Variants

In some embodiments, the disclosed prime editors may comprise a catalytically inactive, or “dead,” napDNAbp domain. In certain embodiments, the prime editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivation of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1 (SEQ ID NO: 39)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1 (SEQ ID NO: 39)) are provided having amino acid sequences which are shorter, or longer than NC_017053.1 (SEQ ID NO: 39) by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a D10X and an H810X, wherein X may be any amino acid, substitutions (underlined and bolded), or a variant be variant of SEQ ID NO: 57 having at least 80%, at least 85%, at least 90%, at least 95%, or at least 99% sequence identity thereto.

In one embodiment, the dead Cas9 may be based on the canonical SpCas9 sequence of Q99ZW2 and may have the following sequence, which comprises a D1LA and an H81A substitutions (underlined and bolded), or be a variant of SEQ ID NO: 58 having at least 80%, at least 85%, at least 90%, at least 95%, or at least 99% sequence identity thereto.

| Description | Sequence | SEQ ID NO: |
|---|---|---|
| dead Cas9 or | MDKKYSIGLXIGTNSVGWAVITDEYKVPSKKFKVLGNTD | SEQ ID NO: 57 |
| dCas9 | RHSIKKNLIGALLFDSGETAEATRLKRTARRRYTRRKNRI | |
| Streptococcus | CYLQEIFSNEMAKVDDSFFHRLEESFLVEEDKKHERHPIF | |
| pyogenes | GNIVDEVAYHEKYPTIYHLRKKLVDSTDKADLRLIYLAL | |
| Q99ZW2 | AHMIKFRGHFLIEGDLNPDNSDVDKLFIQLVQTYNQLFEE | |
| Cas9 with | NPINASGVDAKAILSARLSKSRRLENLIAQLPGEKKNGLF | |
| D10X and | GNLIALSLGLTPNFKSNFDLAEDAKLQLSKDTYDDDLDN | |
| H810X | LLAQIGDQYADLFLAAKNLSDAILLSDILRVNTEITKAPLS | |
| Where “X” is | ASMIKRYDEHHQDLTLLKALVRQQLPEKYKEIFFDQSKN | |
| any amino | GYAGYIDGGASQEEFYKFIKPILEKMDGTEELLVKLNRE | |
| acid | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDXIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| dead Cas9 or | MDKKYSIGLAIGTNSVGWAVITDEYKVPSKKFKVLGNTD | SEQ ID NO: 58 |
| dCas9 | RHSIKKNLIGALLFDSGETAEATRLKRTARRRYTRRKNRI | |
| Streptococcus | CYLQEIFSNEMAKVDDSFFHRLEESFLVEEDKKHERHPIF | |
| pyogenes | GNIVDEVAYHEKYPTIYHLRKKLVDSTDKADLRLIYLAL | |
| Q99ZW2 | AHMIKFRGHFLIEGDLNPDNSDVDKLFIQLVQTYNQLFEE | |
| Cas9 with | NPINASGVDAKAILSARLSKSRRLENLIAQLPGEKKNGLF | |
| D10A and | GNLIALSLGLTPNFKSNFDLAEDAKLQLSKDTYDDDLDN | |
| H810A | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDAIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

D. napDNAbp Nickase Variants

In some embodiments, the disclosed base editors may comprise a napDNAbp domain that comprises a nickase. In one embodiment, the prime editors described herein comprise a Cas9 nickase. The term “Cas9 nickase” or “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include DOX, H983X, D986X, or E762X, wherein X is any amino acid other than the wild type amino acid. In certain embodiments, the nickase could be D1A, of H983A, or D986A, or E762A, or a combination thereof.

In various embodiments, the Cas9 nickase can have a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLXIGTNSVGWAVITDEYKVPSKKFKVLGNTD | SEQ ID NO: 59 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| D10X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 60 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| E762X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIXMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 61 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H983X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHX | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 62 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| D986X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHXAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLAIGTNSVGWAVITDEYKVPSKKFKVLGNTD | SEQ ID NO: 63 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| D10A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 64 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| E762A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIAMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 65 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H983A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHA | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 66 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| D986A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHAAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLDIGTNSVGWAVITDEYKVPSKKFKVLGNTD | SEQ ID NO: 67 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H840X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDXIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 68 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H840A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDAIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 69 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| R863X, | NPINASGVDAKAILSARLSKSRRLENLIAQLPGEKKNGLF | |
| wherein X is | GNLIALSLGLTPNFKSNFDLAEDAKLQLSKDTYDDDLDN | |
| any alternate | LLAQIGDQYADLFLAAKNLSDAILLSDILRVNTEITKAPLS | |
| amino acid | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNXGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |
| Cas9 nickase | MDKKYSIGLDIGTNSVGWAVITDEYKVPSKKFKVLGNTD | SEQ ID NO: 70 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| R863A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDHIVPQSFLKDDSIDNKVLTRSDKNAGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | DKKYSIGLDIGTNSVGWAVITDEYKVPSKKFKVLGNTDR | SEQ ID NO: 71 |
| (Met minus) | HSIKKNLIGALLFDSGETAEATRLKRTARRRYTRRKNRIC | |
| Streptococcus | YLQEIFSNEMAKVDDSFFHRLEESFLVEEDKKHERHPIFG | |
| pyogenes | NIVDEVAYHEKYPTIYHLRKKLVDSTDKADLRLIYLALA | |
| Q99ZW2 | HMIKFRGHFLIEGDLNPDNSDVDKLFIQLVQTYNQLFEE | |
| Cas9 with | NPINASGVDAKAILSARLSKSRRLENLIAQLPGEKKNGLF | |
| H840X, | GNLIALSLGLTPNFKSNFDLAEDAKLQLSKDTYDDDLDN | |
| wherein X is | LLAQIGDQYADLFLAAKNLSDAILLSDILRVNTEITKAPL | |
| any alternate | SASMIKRYDEHHQDLTLLKALVRQQLPEKYKEIFFDQSK | |
| amino acid | NGYAGYIDGGASQEEFYKFIKPILEKMDGTEELLVKLNR | |
| | EDLLRKQRTFDNGSIPHQIHLGELHAILRRQEDFYPFLKD | |
| | NREKIEKILTFRIPYYVGPLARGNSRFAWMTRKSEETITP | |
| | WNFEEVVDKGASAQSFIERMTNFDKNLPNEKVLPKHSL | |
| | LYEYFTVYNELTKVKYVTEGMRKPAFLSGEQKKAIVDL | |
| | LFKTNRKVTVKQLKEDYFKKIECFDSVEISGVEDRFNAS | |
| | LGTYHDLLKIIKDKDFLDNEENEDILEDIVLTLTLFEDRE | |
| | MIEERLKTYAHLFDDKVMKQLKRRRYTGWGRLSRKLIN | |
| | GIRDKQSGKTILDFLKSDGFANRNFMQLIHDDSLTFKEDI | |
| | QKAQVSGQGDSLHEHIANLAGSPAIKKGILQTVKVVDEL | |
| | VKVMGRHKPENIVIEMARENQTTQKGQKNSRERMKRIE | |
| | EGIKELGSQILKEHPVENTQLQNEKLYLYYLQNGRDMY | |
| | VDQELDINRLSDYDVDXIVPQSFLKDDSIDNKVLTRSDK | |
| | NRGKSDNVPSEEVVKKMKNYWRQLLNAKLITQRKFDN | |
| | LTKAERGGLSELDKAGFIKRQLVETRQITKHVAQILDSR | |
| | MNTKYDENDKLIREVKVITLKSKLVSDFRKDFQFYKVRE | |
| | INNYHHAHDAYLNAVVGTALIKKYPKLESEFVYGDYKV | |
| | YDVRKMIAKSEQEIGKATAKYFFYSNIMNFFKTEITLAN | |
| | GEIRKRPLIETNGETGEIVWDKGRDFATVRKVLSMPQVN | |
| | IVKKTEVQTGGFSKESILPKRNSDKLIARKKDWDPKKYG | |
| | GFDSPTVAYSVLVVAKVEKGKSKKLKSVKELLGITIMER | |
| | SSFEKNPIDFLEAKGYKEVKKDLIIKLPKYSLFELENGRK | |
| | RMLASAGELQKGNELALPSKYVNFLYLASHYEKLKGSP | |
| | EDNEQKQLFVEQHKHYLDEIIEQISEFSKRVILADANLDK | |
| | VLSAYNKHRDKPIREQAENIIHLFTLTNLGAPAAFKYFDT | |
| | TIDRKRYTSTKEVLDATLIHQSITGLYETRIDLSQLGGD | |
| Cas9 nickase | DKKYSIGLDIGTNSVGWAVITDEYKVPSKKFKVLGNTDR | SEQ ID NO: 31 |
| (Met minus) | HSIKKNLIGALLFDSGETAEATRLKRTARRRYTRRKNRIC | |
| Streptococcus | YLQEIFSNEMAKVDDSFFHRLEESFLVEEDKKHERHPIFG | |
| pyogenes | NIVDEVAYHEKYPTIYHLRKKLVDSTDKADLRLIYLALA | |
| Q99ZW2 | HMIKFRGHFLIEGDLNPDNSDVDKLFIQLVQTYNQLFEE | |
| Cas9 with | NPINASGVDAKAILSARLSKSRRLENLIAQLPGEKKNGLF | |
| H840A | GNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSK | |
| | NGYAGYIDGGASQEEFYKFIKPILEKMDGTEELLVKLNR | |
| | EDLLRKQRTFDNGSIPHQIHLGELHAILRRQEDFYPFLKD | |
| | NREKIEKILTFRIPYYVGPLARGNSRFAWMTRKSEETITP | |
| | WNFEEVVDKGASAQSFIERMTNFDKNLPNEKVLPKHSL | |
| | LYEYFTVYNELTKVKYVTEGMRKPAFLSGEQKKAIVDL | |
| | LFKTNRKVTVKQLKEDYFKKIECFDSVEISGVEDRFNAS | |
| | LGTYHDLLKIIKDKDFLDNEENEDILEDIVLTLTLFEDRE | |
| | MIEERLKTYAHLFDDKVMKQLKRRRYTGWGRLSRKLIN | |
| | GIRDKQSGKTILDFLKSDGFANRNFMQLIHDDSLTFKEDI | |
| | QKAQVSGQGDSLHEHIANLAGSPAIKKGILQTVKVVDEL | |
| | VKVMGRHKPENIVIEMARENQTTQKGQKNSRERMKRIE | |
| | EGIKELGSQILKEHPVENTQLQNEKLYLYYLQNGRDMY | |
| | VDQELDINRLSDYDVDAIVPQSFLKDDSIDNKVLTRSDK | |
| | NRGKSDNVPSEEVVKKMKNYWRQLLNAKLITQRKFDN | |
| | LTKAERGGLSELDKAGFIKRQLVETRQITKHVAQILDSR | |
| | MNTKYDENDKLIREVKVITLKSKLVSDFRKDFQFYKVRE | |
| | INNYHHAHDAYLNAVVGTALIKKYPKLESEFVYGDYKV | |
| | YDVRKMIAKSEQEIGKATAKYFFYSNIMNFFKTEITLAN | |
| | GEIRKRPLIETNGETGEIVWDKGRDFATVRKVLSMPQVN | |
| | IVKKTEVQTGGFSKESILPKRNSDKLIARKKDWDPKKYG | |
| | GFDSPTVAYSVLVVAKVEKGKSKKLKSVKELLGITIMER | |
| | SSFEKNPIDFLEAKGYKEVKKDLIIKLPKYSLFELENGRK | |
| | RMLASAGELQKGNELALPSKYVNFLYLASHYEKLKGSP | |
| | EDNEQKQLFVEQHKHYLDEIIEQISEFSKRVILADANLDK | |
| | VLSAYNKHRDKPIREQAENIIHLFTLTNLGAPAAFKYFDT | |
| | TIDRKRYTSTKEVLDATLIHQSITGLYETRIDLSQLGGD | |
| Cas9 nickase | DKKYSIGLDIGTNSVGWAVITDEYKVPSKKFKVLGNTDR | SEQ ID NO: 72 |
| (Met minus) | HSIKKNLIGALLFDSGETAEATRLKRTARRRYTRRKNRIC | |
| Streptococcus | YLQEIFSNEMAKVDDSFFHRLEESFLVEEDKKHERHPIFG | |
| pyogenes | NIVDEVAYHEKYPTIYHLRKKLVDSTDKADLRLIYLALA | |
| Q99ZW2 | HMIKFRGHFLIEGDLNPDNSDVDKLFIQLVQTYNQLFEE | |
| Cas9 with | NPINASGVDAKAILSARLSKSRRLENLIAQLPGEKKNGLF | |
| R863X, | GNLIALSLGLTPNFKSNFDLAEDAKLQLSKDTYDDDLDN | |
| wherein X is | LLAQIGDQYADLFLAAKNLSDAILLSDILRVNTEITKAPL | |
| any alternate | SASMIKRYDEHHQDLTLLKALVRQQLPEKYKEIFFDQSK | |
| amino acid | NGYAGYIDGGASQEEFYKFIKPILEKMDGTEELLVKLNR | |
| | EDLLRKQRTFDNGSIPHQIHLGELHAILRRQEDFYPFLKD | |
| | NREKIEKILTFRIPYYVGPLARGNSRFAWMTRKSEETITP | |
| | WNFEEVVDKGASAQSFIERMTNFDKNLPNEKVLPKHSL | |
| | LYEYFTVYNELTKVKYVTEGMRKPAFLSGEQKKAIVDL | |
| | LFKTNRKVTVKQLKEDYFKKIECFDSVEISGVEDRFNAS | |
| | LGTYHDLLKIIKDKDFLDNEENEDILEDIVLTLTLFEDRE | |
| | MIEERLKTYAHLFDDKVMKQLKRRRYTGWGRLSRKLIN | |
| | GIRDKQSGKTILDFLKSDGFANRNFMQLIHDDSLTFKEDI | |
| | QKAQVSGQGDSLHEHIANLAGSPAIKKGILQTVKVVDEL | |
| | VKVMGRHKPENIVIEMARENQTTQKGQKNSRERMKRIE | |
| | EGIKELGSQILKEHPVENTQLQNEKLYLYYLQNGRDMY | |
| | VDQELDINRLSDYDVDHIVPQSFLKDDSIDNKVLTRSDK | |
| | NXGKSDNVPSEEVVKKMKNYWRQLLNAKLITQRKFDN | |
| | LTKAERGGLSELDKAGFIKRQLVETRQITKHVAQILDSR | |
| | MNTKYDENDKLIREVKVITLKSKLVSDFRKDFQFYKVRE | |
| | INNYHHAHDAYLNAVVGTALIKKYPKLESEFVYGDYKV | |
| | YDVRKMIAKSEQEIGKATAKYFFYSNIMNFFKTEITLAN | |
| | GEIRKRPLIETNGETGEIVWDKGRDFATVRKVLSMPQVN | |
| | IVKKTEVQTGGFSKESILPKRNSDKLIARKKDWDPKKYG | |
| | GFDSPTVAYSVLVVAKVEKGKSKKLKSVKELLGITIMER | |
| | SSFEKNPIDFLEAKGYKEVKKDLIIKLPKYSLFELENGRK | |
| | RMLASAGELQKGNELALPSKYVNFLYLASHYEKLKGSP | |
| | EDNEQKQLFVEQHKHYLDEIIEQISEFSKRVILADANLDK | |
| | VLSAYNKHRDKPIREQAENIIHLFTLTNLGAPAAFKYFDT | |
| | TIDRKRYTSTKEVLDATLIHQSITGLYETRIDLSQLGGD | |
| Cas9 nickase | DKKYSIGLDIGTNSVGWAVITDEYKVPSKKFKVLGNTDR | SEQ ID NO: 73 |
| (Met minus) | HSIKKNLIGALLFDSGETAEATRLKRTARRRYTRRKNRIC | |
| Streptococcus | YLQEIFSNEMAKVDDSFFHRLEESFLVEEDKKHERHPIFG | |
| pyogenes | NIVDEVAYHEKYPTIYHLRKKLVDSTDKADLRLIYLALA | |
| Q99ZW2 | HMIKFRGHFLIEGDLNPDNSDVDKLFIQLVQTYNQLFEE | |
| Cas9 with | NPINASGVDAKAILSARLSKSRRLENLIAQLPGEKKNGLF | |
| R863A | GNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSK | |
| | NGYAGYIDGGASQEEFYKFIKPILEKMDGTEELLVKLNR | |
| | EDLLRKQRTFDNGSIPHQIHLGELHAILRRQEDFYPFLKD | |
| | NREKIEKILTFRIPYYVGPLARGNSRFAWMTRKSEETITP | |
| | WNFEEVVDKGASAQSFIERMTNFDKNLPNEKVLPKHSL | |
| | LYEYFTVYNELTKVKYVTEGMRKPAFLSGEQKKAIVDL | |
| | LFKTNRKVTVKQLKEDYFKKIECFDSVEISGVEDRFNAS | |
| | LGTYHDLLKIIKDKDFLDNEENEDILEDIVLTLTLFEDRE | |
| | MIEERLKTYAHLFDDKVMKQLKRRRYTGWGRLSRKLIN | |
| | GIRDKQSGKTILDFLKSDGFANRNFMQLIHDDSLTFKEDI | |
| | QKAQVSGQGDSLHEHIANLAGSPAIKKGILQTVKVVDEL | |
| | VKVMGRHKPENIVIEMARENQTTQKGQKNSRERMKRIE | |
| | EGIKELGSQILKEHPVENTQLQNEKLYLYYLQNGRDMY | |
| | VDQELDINRLSDYDVDHIVPQSFLKDDSIDNKVLTRSDK | |
| | NAGKSDNVPSEEVVKKMKNYWRQLLNAKLITQRKFDN | |
| | LTKAERGGLSELDKAGFIKRQLVETRQITKHVAQILDSR | |
| | MNTKYDENDKLIREVKVITLKSKLVSDFRKDFQFYKVRE | |
| | INNYHHAHDAYLNAVVGTALIKKYPKLESEFVYGDYKV | |
| | YDVRKMIAKSEQEIGKATAKYFFYSNIMNFFKTEITLAN | |
| | GEIRKRPLIETNGETGEIVWDKGRDFATVRKVLSMPQVN | |
| | IVKKTEVQTGGFSKESILPKRNSDKLIARKKDWDPKKYG | |
| | GFDSPTVAYSVLVVAKVEKGKSKKLKSVKELLGITIMER | |
| | SSFEKNPIDFLEAKGYKEVKKDLIIKLPKYSLFELENGRK | |
| | RMLASAGELQKGNELALPSKYVNFLYLASHYEKLKGSP | |
| | EDNEQKQLFVEQHKHYLDEIIEQISEFSKRVILADANLDK | |
| | VLSAYNKHRDKPIREQAENIIHLFTLTNLGAPAAFKYFDT | |
| | TIDRKRYTSTKEVLDATLIHQSITGLYETRIDLSQLGGD | |

### E. Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 37).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### F. Small-Sized Cas9 Variants

In some embodiments, the prime editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. In certain embodiments, the smaller-sized Cas9 variants can include enzymes categorized as type II enzymes of the Class 2 CRISPR-Cas systems. In some embodiments, the smaller-sized Cas9 variants can include enzymes categorized as type V enzymes of the Class 2 CRISPR-Cas systems. In other embodiments, the smaller-sized Cas9 variants can include enzymes categorized as type VI enzymes of the Class 2 CRISPR-Cas systems.

The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein. The Cas9 variants can include those categorized as type II, type V, or type VI enzymes of the Class 2 CRISPR-Cas system.

In various embodiments, the prime editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| Description | Sequence | SEQ ID NO: |
|---|---|---|
| SaCas9 | MGKRNYILGLDIGITSVGYGIIDYETRDVIDAGVRLFKEA | SEQ ID NO: |
| Staphylococcus | NVENNEGRRSKRGARRLKRRRRHRIQRVKKLLFDYNLL | 45 |
| aureus | TDHSELSGINPYEARVKGLSQKLSEEEFSAALLHLAKRRG | |
| 1053 AA | VHNVNEVEEDTGNELSTKEQISRNSKALEEKYVAELQLE | |
| 123 kDa | RLKKDGEVRGSINRFKTSDYVKEAKQLLKVQKAYHQLD | |
| | QSFIDTYIDLLETRRTYYEGPGEGSPFGWKDIKEWYEML | |
| | MGHCTYFPEELRSVKYAYNADLYNALNDLNNLVITRDE | |
| | NEKLEYYEKFQIIENVFKQKKKPTLKQIAKEILVNEEDIK | |
| | GYRVTSTGKPEFTNLKVYHDIKDITARKEIIENAELLDQIA | |
| | KILTIYQSSEDIQEELTNLNSELTQEEIEQISNLKGYTGTHN | |
| | LSLKAINLILDELWHTNDNQIAIFNRLKLVPKKVDLSQQK | |
| | EIPTTLVDDFILSPVVKRSFIQSIKVINAIIKKYGLPNDIIIEL | |
| | AREKNSKDAQKMINEMQKRNRQTNERIEEIIRTTGKENA | |
| | KYLIEKIKLHDMQEGKCLYSLEAIPLEDLLNNPFNYEVDH | |
| | IIPRSVSFDNSFNNKVLVKQEENSKKGNRTPFQYLSSSDS | |
| | KISYETFKKHILNLAKGKGRISKTKKEYLLEERDINRFSV | |
| | QKDFINRNLVDTRYATRGLMNLLRSYFRVNNLDVKVKSI | |
| | NGGFTSFLRRKWKFKKERNKGYKHHAEDALIIANADFIF | |
| | KEWKKLDKAKKVMENQMFEEKQAESMPEIETEQEYKEI | |
| | FITPHQIKHIKDFKDYKYSHRVDKKPNRKLINDTLYSTRK | |
| | DDKGNTLIVNNLNGLYDKDNDKLKKLINKSPEKLLMYH | |
| | HDPQTYQKLKLIMEQYGDEKNPLYKYYEETGNYLTKYS | |
| | KKDNGPVIKKIKYYGNKLNAHLDITDDYPNSRNKVVKLS | |
| | LKPYRFDVYLDNGVYKFVTVKNLDVIKKENYYEVNSKC | |
| | YEEAKKLKKISNQAEFIASFYKNDLIKINGELYRVIGVNN | |
| | DLLNRIEVNMIDITYREYLENMNDKRPPHIIKTIASKTQSI | |
| | KKYSTDILGNLYEVKSKKHPQIIKK | |
| NmeCas9 | MAAFKPNSINYILGLDIGIASVGWAMVEIDEEENPIRLIDL | SEQ ID NO: |
| N. | GVRVFERAEVPKTGDSLAMARRLARSVRRLTRRRAHRL | 74 |
| meningitidis | LRTRRLLKREGVLQAANFDENGLIKSLPNTPWQLRAAAL | |
| 1083 AA | DRKLTPLEWSAVLLHLIKHRGYLSQRKNEGETADKELGA | |
| 124.5 kDa | LLKGVAGNAHALQTGDFRTPAELALNKFEKESGHIRNQR | |
| | SDYSHTFSRKDLQAELILLFEKQKEFGNPHVSGGLKEGIE | |
| | TLLMTQRPALSGDAVQKMLGHCTFEPAEPKAAKNTYTA | |
| | ERFIWLTKLNNLRILEQGSERPLTDTERATLMDEPYRKSK | |
| | LTYAQARKLLGLEDTAFFKGLRYGKDNAEASTLMEMKA | |
| | YHAISRALEKEGLKDKKSPLNLSPELQDEIGTAFSLFKTD | |
| | EDITGRLKDRIQPEILEALLKHISFDKFVQISLKALRRIVPL | |
| | MEQGKRYDEACAEIYGDHYGKKNTEEKIYLPPIPADEIR | |
| | NPVVLRALSQARKVINGVVRRYGSPARIHIETAREVGKSF | |
| | KDRKEIEKRQEENRKDREKAAAKFREYFPNFVGEPKSKD | |
| | ILKLRLYEQQHGKCLYSGKEINLGRLNEKGYVEIDAALPF | |
| | SRTWDDSFNNKVLVLGSENQNKGNQTPYEYFNGKDNSR | |
| | EWQEFKARVETSRFPRSKKQRILLQKFDEDGFKERNLND | |
| | TRYVNRFLCQFVADRMRLTGKGKKRVFASNGQITNLLR | |
| | GFWGLRKVRAENDRHHALDAVVVACSTVAMQQKITRF | |
| | VRYKEMNAFDGKTIDKETGEVLHQKTHFPQPWEFFAQE | |
| | VMIRVFGKPDGKPEFEEADTLEKLRTLLAEKLSSRPEAVH | |
| | EYVTPLFVSRAPNRKMSGQGHMETVKSAKRLDEGVSVL | |
| | RVPLTQLKLKDLEKMVNREREPKLYEALKARLEAHKDD | |
| | PAKAFAEPFYKYDKAGNRTQQVKAVRVEQVQKTGVWV | |
| | RNHNGIADNATMVRVDVFEKGDKYYLVPIYSWQVAKGI | |
| | LPDRAVVQGKDEEDWQLIDDSFNFKFSLHPNDLVEVITK | |
| | KARMFGYFASCHRGTGNINIRIHDLDHKIGKNGILEGIGV | |
| | KTALSFQKYQIDELGKEIRPCRLKKRPPVR | |
| CjCas9 | MARILAFDIGISSIGWAFSENDELKDCGVRIFTKVENPKT | SEQ ID NO: |
| C. jejuni | GESLALPRRLARSARKRLARRKARLNHLKHLIANEFKLN | 75 |
| 984 AA | YEDYQSFDESLAKAYKGSLISPYELRFRALNELLSKQDFA | |
| 114.9 kDa | RVILHIAKRRGYDDIKNSDDKEKGAILKAIKQNEEKLAN | |
| | YQSVGEYLYKEYFQKFKENSKEFTNVRNKKESYERCIAQ | |
| | SFLKDELKLIFKKQREFGFSFSKKFEEEVLSVAFYKRALK | |
| | DFSHLVGNCSFFTDEKRAPKNSPLAFMFVALTRIINLLNN | |
| | LKNTEGILYTKDDLNALLNEVLKNGTLTYKQTKKLLGLS | |
| | DDYEFKGEKGTYFIEFKKYKEFIKALGEHNLSQDDLNEIA | |
| | KDITLIKDEIKLKKALAKYDLNQNQIDSLSKLEFKDHLNIS | |
| | FKALKLVTPLMLEGKKYDEACNELNLKVAINEDKKDFLP | |
| | AFNETYYKDEVTNPVVLRAIKEYRKVLNALLKKYGKVH | |
| | KINIELAREVGKNHSQRAKIEKEQNENYKAKKDAELECE | |
| | KLGLKINSKNILKLRLFKEQKEFCAYSGEKIKISDLQDEK | |
| | MLEIDHIYPYSRSFDDSYMNKVLVFTKQNQEKLNQTPFE | |
| | AFGNDSAKWQKIEVLAKNLPTKKQKRILDKNYKDKEQK | |
| | NFKDRNLNDTRYIARLVLNYTKDYLDFLPLSDDENTKLN | |
| | DTQKGSKVHVEAKSGMLTSALRHTWGFSAKDRNNHLH | |
| | HAIDAVIIAYANNSIVKAFSDFKKEQESNSAELYAKKISEL | |
| | DYKNKRKFFEPFSGFRQKVLDKIDEIFVSKPERKKPSGAL | |
| | HEETFRKEEEFYQSYGGKEGVLKALELGKIRKVNGKIVK | |
| | NGDMFRVDIFKHKKTNKFYAVPIYTMDFALKVLPNKAV | |
| | ARSKKGEIKDWILMDENYEFCFSLYKDSLILIQTKDMQEP | |
| | EFVYYNAFTSSTVSLIVSKHDNKFETLSKNQKILFKNANE | |
| | KEVIAKSIGIQNLKVFEKYIVSALGEVTKAEFRQREDFKK | |
| GeoCas9 | MRYKIGLDIGITSVGWAVMNLDIPRIEDLGVRIFDRAENP | SEQ ID NO: |
| G. | QTGESLALPRRLARSARRRLRRRKHRLERIRRLVIREGILT | 76 |
| stearothermo- | KEELDKLFEEKHEIDVWQLRVEALDRKLNNDELARVLL | |
| philus | HLAKRRGFKSNRKSERSNKENSTMLKHIEENRAILSSYRT | |
| 1087 AA | VGEMIVKDPKFALHKRNKGENYTNTIARDDLEREIRLIFS | |
| 127 kDa | KQREFGNMSCTEEFENEYITIWASQRPVASKDDIEKKVGF | |
| | CTFEPKEKRAPKATYTFQSFIAWEHINKLRLISPSGARGLT | |
| | DEERRLLYEQAFQKNKITYHDIRTLLHLPDDTYFKGIVYD | |
| | RGESRKQNENIRFLELDAYHQIRKAVDKVYGKGKSSSFL | |
| | PIDFDTFGYALTLFKDDADIHSYLRNEYEQNGKRMPNLA | |
| | NKVYDNELIEELLNLSFTKFGHLSLKALRSILPYMEQGEV | |
| | YSSACERAGYTFTGPKKKQKTMLLPNIPPIANPVVMRAL | |
| | TQARKVVNAIIKKYGSPVSIHIELARDLSQTFDERRKTKK | |
| | EQDENRKKNETAIRQLMEYGLTLNPTGHDIVKFKLWSEQ | |
| | NGRCAYSLQPIEIERLLEPGYVEVDHVIPYSRSLDDSYTN | |
| | KVLVLTRENREKGNRIPAEYLGVGTERWQQFETFVLTNK | |
| | QFSKKKRDRLLRLHYDENEETEFKNRNLNDTRYISRFFA | |
| | NFIREHLKFAESDDKQKVYTVNGRVTAHLRSRWEFNKN | |
| | REESDLHHAVDAVIVACTTPSDIAKVTAFYQRREQNKEL | |
| | AKKTEPHFPQPWPHFADELRARLSKHPKESIKALNLGNY | |
| | DDQKLESLQPVFVSRMPKRSVTGAAHQETLRRYVGIDER | |
| | SGKIQTVVKTKLSEIKLDASGHFPMYGKESDPRTYEAIRQ | |
| | RLLEHNNDPKKAFQEPLYKPKKNGEPGPVIRTVKIIDTKN | |
| | QVIPLNDGKTVAYNSNIVRVDVFEKDGKYYCVPVYTMD | |
| | IMKGILPNKAIEPNKPYSEWKEMTEDYTFRFSLYPNDLIRI | |
| | ELPREKTVKTAAGEEINVKDVFVYYKTIDSANGGLELISH | |
| | DHRFSLRGVGSRTLKRFEKYQVDVLGNIYKVRGEKRVG | |
| | LASSAHSKPGKTIRPLQSTRD | |
| LbaCas 12a | MSKLEKFTNCYSLSKTLRFKAIPVGKTQENIDNKRLLVED | SEQ ID NO: |
| L. bacterium | EKRAEDYKGVKKLLDRYYLSFINDVLHSIKLKNLNNYIS | 77 |
| 1228 AA | LFRKKTRTEKENKELENLEINLRKEIAKAFKGNEGYKSLF | |
| 143.9 kDa | KKDIIETILPEFLDDKDEIALVNSFNGFTTAFTGFFDNREN | |
| | MFSEEAKSTSIAFRCINENLTRYISNMDIFEKVDAIFDKHE | |
| | VQEIKEKILNSDYDVEDFFEGEFFNFVLTQEGIDVYNAIIG | |
| | GFVTESGEKIKGLNEYINLYNQKTKQKLPKFKPLYKQVL | |
| | SDRESLSFYGEGYTSDEEVLEVFRNTLNKNSEIFSSIKKLE | |
| | KLFKNFDEYSSAGIFVKNGPAISTISKDIFGEWNVIRDKW | |
| | NAEYDDIHLKKKAVVTEKYEDDRRKSFKKIGSFSLEQLQ | |
| | EYADADLSVVEKLKEIIIQKVDEIYKVYGSSEKLFDADFV | |
| | LEKSLKKNDAVVAIMKDLLDSVKSFENYIKAFFGEGKET | |
| | NRDESFYGDFVLAYDILLKVDHIYDAIRNYVTQKPYSKD | |
| | KFKLYFQNPQFMGGWDKDKETDYRATILRYGSKYYLAI | |
| | MDKKYAKCLQKIDKDDVNGNYEKINYKLLPGPNKMLPK | |
| | VFFSKKWMAYYNPSEDIQKIYKNGTFKKGDMFNLNDCH | |
| | KLIDFFKDSISRYPKWSNAYDFNFSETEKYKDIAGFYREV | |
| | EEQGYKVSFESASKKEVDKLVEEGKLYMFQIYNKDFSDK | |
| | SHGTPNLHTMYFKLLFDENNHGQIRLSGGAELFMRRASL | |
| | KKEELVVHPANSPIANKNPDNPKKTTTLSYDVYKDKRFS | |
| | EDQYELHIPIAINKCPKNIFKINTEVRVLLKHDDNPYVIGI | |
| | DRGERNLLYIVVVDGKGNIVEQYSLNEIINNFNGIRIKTD | |
| | YHSLLDKKEKERFEARQNWTSIENIKELKAGYISQVVHKI | |
| | CELVEKYDAVIALEDLNSGFKNSRVKVEKQVYQKFEKM | |
| | LIDKLNYMVDKKSNPCATGGALKGYQITNKFESFKSMST | |
| | QNGFIFYIPAWLTSKIDPSTGFVNLLKTKYTSIADSKKFISS | |
| | FDRIMYVPEEDLFEFALDYKNFSRTDADYIKKWKLYSYG | |
| | NRIRIFRNPKKNNVFDWEEVCLTSAYKELFNKYGINYQQ | |
| | GDIRALLCEQSDKAFYSSFMALMSLMLQMRNSITGRTDV | |
| | DFLISPVKNSDGIFYDSRNYEAQENAILPKNADANGAYNI | |
| | ARKVLWAIGQFKKAEDEKLDKVKIAISNKEWLEYAQTS | |
| | VKH | |
| BhCas12b | MATRSFILKIEPNEEVKKGLWKTHEVLNHGIAYYMNILK | SEQ ID NO: |
| B. hisashii | LIRQEAIYEHHEQDPKNPKKVSKAEIQAELWDFVLKMQK | 78 |
| 1108 AA | CNSFTHEVDKDEVFNILRELYEELVPSSVEKKGEANQLSN | |
| 130.4 kDa | KFLYPLVDPNSQSGKGTASSGRKPRWYNLKIAGDPSWEE | |
| | EKKKWEEDKKKDPLAKILGKLAEYGLIPLFIPYTDSNEPI | |
| | VKEIKWMEKSRNQSVRRLDKDMFIQALERFLSWESWNL | |
| | KVKEEYEKVEKEYKTLEERIKEDIQALKALEQYEKERQE | |
| | QLLRDTLNTNEYRLSKRGLRGWREIIQKWLKMDENEPSE | |
| | KYLEVFKDYQRKHPREAGDYSVYEFLSKKENHFIWRNH | |
| | PEYPYLYATFCEIDKKKKDAKQQATFTLADPINHPLWVR | |
| | FEERSGSNLNKYRILTEQLHTEKLKKKLTVQLDRLIYPTE | |
| | SGGWEEKGKVDIVLLPSRQFYNQIFLDIEEKGKHAFTYK | |
| | DESIKFPLKGTLGGARVQFDRDHLRRYPHKVESGNVGRI | |
| | YFNMTVNIEPTESPVSKSLKIHRDDFPKVVNFKPKELTEW | |
| | IKDSKGKKLKSGIESLEIGLRVMSIDLGQRQAAAASIFEV | |
| | VDQKPDIEGKLFFPIKGTELYAVHRASFNIKLPGETLVKS | |
| | REVLRKAREDNLKLMNQKLNFLRNVLHFQQFEDITEREK | |
| | RVTKWISRQENSDVPLVYQDELIQIRELMYKPYKDWVAF | |
| | LKQLHKRLEVEIGKEVKHWRKSLSDGRKGLYGISLKNID | |
| | EIDRTRKFLLRWSLRPTEPGEVRRLEPGQRFAIDQLNHLN | |
| | ALKEDRLKKMANTIIMHALGYCYDVRKKKWQAKNPAC | |
| | QIILFEDLSNYNPYEERSRFENSKLMKWSRREIPRQVALQ | |
| | GEIYGLQVGEVGAQFSSRFHAKTGSPGIRCSVVTKEKLQ | |
| | DNRFFKNLQREGRLTLDKIAVLKEGDLYPDKGGEKFISLS | |
| | KDRKCVTTHADINAAQNLQKRFWTRTHGFYKVYCKAY | |
| | QVDGQTVYIPESKDQKQKIIEEFGEGYFILKDGVYEWVN | |
| | AGKLKIKKGSSKQSSSELVDSDILKDSFDLASELKGEKLM | |
| | LYRDPSGNVFPSDKWMAAGVFFGKLERILISKLTNQYSIS | |
| | TIEDDSSKQSM | |

### G. Cas9 Equivalents

In some embodiments, the prime editors described herein can include any Cas9 equivalent. As used herein, the term “Cas9 equivalent” is a broad term that encompasses any napDNAbp protein that serves the same function as Cas9 in the present prime editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The prime editors described here embrace any Cas9 equivalent that would provide the same or similar function as Cas9 despite that the Cas9 equivalent may be based on a protein that arose through convergent evolution. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein described in Liu et al., “CasX enzymes comprises a distinct family of RNA-guided genome editors,” Nature, 2019, Vol. 566: 218-223, is contemplated to be used with the prime editors described herein. In addition, any variant or modification of Cas12e (CasX) is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to Cas12e (CasX) or Cas12d (CasY), which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the napDNAbp is a naturally-occurring Cas12e (CasX) or Cas12d (CasY) protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), Cas12e (CasX), Cas12d (CasY), Cas12a (Cpf1), Cas12b1 (C2c1), Cas13a (C2c2), Cas12c (C2c3), Argonaute, and Cas12b1. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

In still other embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmnr1, Cmnr3, Cmnr4, Cmnr5, Cmnr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 37).

In various other embodiments, the napDNAbp can be any of the following proteins: a Cas9, a Cas12a (Cpf1), a Cas12e (CasX), a CasL2d (CasY), a Cas2bE (C2c), a Cas13a (C2c2), a Cas12c (C2c3), a GeoCas9, a CjCas9, a Cas12g, a Cas12h, a Cas12i, a Cas13b, a Cas113c, a Cas113d, a Cas 14, a Csn2, an xCas9, an SpCas9-NG, a circularly permuted Cas9, or an Argonaute (Ago) domain, or a variant thereof.

Exemplary Cas9 equivalent protein sequences can include the following:

| Description | Sequence | SEQ ID NO: |
|---|---|---|
| AsCas12a | MTQFEGFTNLYQVSKTLRFELIPQGKTLKHIQEQGFIEEDKAR | SEQ ID NO: 79 |
| (previously | NDHYKELKPIIDRIYKTYADQCLQLVQLDWENLSAAIDSYRK | |
| known | EKTEETRNALIEEQATYRNAIHDYFIGRTDNLTDAINKRHAEI | |
| as Cpf1) | YKGLFKAELFNGKVLKQLGTVTTTEHENALLRSFDKFTTYFS | |
| Acidaminococcus | GFYENRKNVFSAEDISTAIPHRIVQDNFPKFKENCHIFTRLITA | |
| sp. | VPSLREHFENVKKAIGIFVSTSIEEVFSFPFYNQLLTQTQIDLYN | |
| (strain | QLLGGISREAGTEKIKGLNEVLNLAIQKNDETAHIIASLPHRFIP | |
| BV3L6) | LFKQILSDRNTLSFILEEFKSDEEVIQSFCKYKTLLRNENVLET | |
| UniProtK | AEALFNELNSIDLTHIFISHKKLETISSALCDHWDTLRNALYER | |
| B | RISELTGKITKSAKEKVQRSLKHEDINLQEIISAAGKELSEAFK | |
| U2UMQ6 | QKTSEILSHAHAALDQPLPTTLKKQEEKEILKSQLDSLLGLYH | |
| | LLDWFAVDESNEVDPEFSARLTGIKLEMEPSLSFYNKARNYA | |
| | TKKPYSVEKFKLNFQMPTLASGWDVNKEKNNGAILFVKNGL | |
| | YYLGIMPKQKGRYKALSFEPTEKTSEGFDKMYYDYFPDAAK | |
| | MIPKCSTQLKAVTAHFQTHTTPILLSNNFIEPLEITKEIYDLNNP | |
| | EKEPKKFQTAYAKKTGDQKGYREALCKWIDFTRDFLSKYTK | |
| | TTSIDLSSLRPSSQYKDLGEYYAELNPLLYHISFQRIAEKEIMD | |
| | AVETGKLYLFQIYNKDFAKGHHGKPNLHTLYWTGLFSPENLA | |
| | KTSIKLNGQAELFYRPKSRMKRMAHRLGEKMLNKKLKDQKT | |
| | PIPDTLYQELYDYVNHRLSHDLSDEARALLPNVITKEVSHEIIK | |
| | DRRFTSDKFFFHVPITLNYQAANSPSKFNQRVNAYLKEHPETP | |
| | IIGIDRGERNLIYITVIDSTGKILEQRSLNTIQQFDYQKKLDNRE | |
| | KERVAARQAWSVVGTIKDLKQGYLSQVIHEIVDLMIHYQAV | |
| | VVLENLNFGFKSKRTGIAEKAVYQQFEKMLIDKLNCLVLKDY | |
| | PAEKVGGVLNPYQLTDQFTSFAKMGTQSGFLFYVPAPYTSKI | |
| | DPLTGFVDPFVWKTIKNHESRKHFLEGFDFLHYDVKTGDFILH | |
| | FKMNRNLSFQRGLPGFMPAWDIVFEKNETQFDAKGTPFIAGK | |
| | RIVPVIENHRFTGRYRDLYPANELIALLEEKGIVFRDGSNILPK | |
| | LLENDDSHAIDTMVALIRSVLQMRNSNAATGEDYINSPVRDL | |
| | NGVCFDSRFQNPEWPMDADANGAYHIALKGQLLLNHLKESK | |
| | DLKLQNGISNQDWLAYIQELRN | |
| AsCas12a | MTQFEGFTNLYQVSKTLRFELIPQGKTLKHIQEQGFIEEDKAR | SEQ ID NO: 80 |
| nickase | NDHYKELKPIIDRIYKTYADQCLQLVQLDWENLSAAIDSYRK | |
| (e.g., | EKTEETRNALIEEQATYRNAIHDYFIGRTDNLTDAINKRHAEI | |
| R1226A) | YKGLFKAELFNGKVLKQLGTVTTTEHENALLRSFDKFTTYFS | |
| | GFYENRKNVFSAEDISTAIPHRIVQDNFPKFKENCHIFTRLITA | |
| | VPSLREHFENVKKAIGIFVSTSIEEVFSFPFYNQLLTQTQIDLYN | |
| | QLLGGISREAGTEKIKGLNEVLNLAIQKNDETAHIIASLPHRFIP | |
| | LFKQILSDRNTLSFILEEFKSDEEVIQSFCKYKTLLRNENVLET | |
| | AEALFNELNSIDLTHIFISHKKLETISSALCDHWDTLRNALYER | |
| | RISELTGKITKSAKEKVQRSLKHEDINLQEIISAAGKELSEAFK | |
| | QKTSEILSHAHAALDQPLPTTLKKQEEKEILKSQLDSLLGLYH | |
| | LLDWFAVDESNEVDPEFSARLTGIKLEMEPSLSFYNKARNYA | |
| | TKKPYSVEKFKLNFQMPTLASGWDVNKEKNNGAILFVKNGL | |
| | YYLGIMPKQKGRYKALSFEPTEKTSEGFDKMYYDYFPDAAK | |
| | MIPKCSTQLKAVTAHFQTHTTPILLSNNFIEPLEITKEIYDLNNP | |
| | EKEPKKFQTAYAKKTGDQKGYREALCKWIDFTRDFLSKYTK | |
| | TTSIDLSSLRPSSQYKDLGEYYAELNPLLYHISFQRIAEKEIMD | |
| | AVETGKLYLFQIYNKDFAKGHHGKPNLHTLYWTGLFSPENLA | |
| | KTSIKLNGQAELFYRPKSRMKRMAHRLGEKMLNKKLKDQKT | |
| | PIPDTLYQELYDYVNHRLSHDLSDEARALLPNVITKEVSHEIIK | |
| | DRRFTSDKFFFHVPITLNYQAANSPSKFNQRVNAYLKEHPETP | |
| | IIGIDRGERNLIYITVIDSTGKILEQRSLNTIQQFDYQKKLDNRE | |
| | KERVAARQAWSVVGTIKDLKQGYLSQVIHEIVDLMIHYQAV | |
| | VVLENLNFGFKSKRTGIAEKAVYQQFEKMLIDKLNCLVLKDY | |
| | PAEKVGGVLNPYQLTDQFTSFAKMGTQSGFLFYVPAPYTSKI | |
| | DPLTGFVDPFVWKTIKNHESRKHFLEGFDFLHYDVKTGDFILH | |
| | FKMNRNLSFQRGLPGFMPAWDIVFEKNETQFDAKGTPFIAGK | |
| | RIVPVIENHRFTGRYRDLYPANELIALLEEKGIVFRDGSNILPK | |
| | LLENDDSHAIDTMVALIRSVLQMANSNAATGEDYINSPVRDL | |
| | NGVCFDSRFQNPEWPMDADANGAYHIALKGQLLLNHLKESK | |
| | DLKLQNGISNQDWLAYIQELRN | |
| LbCas12a | MNYKTGLEDFIGKESLSKTLRNALIPTESTKIHMEEMGVIRDD | SEQ ID NO: 81 |
| (previously | ELRAEKQQELKEIMDDYYRTFIEEKLGQIQGIQWNSLFQKME | |
| known | ETMEDISVRKDLDKIQNEKRKEICCYFTSDKRFKDLFNAKLIT | |
| as Cpf1) | DILPNFIKDNKEYTEEEKAEKEQTRVLFQRFATAFTNYFNQRR | |
| Lachnospiraceae | NNFSEDNISTAISFRIVNENSEIHLQNMRAFQRIEQQYPEEVCG | |
| bacterium | MEEEYKDMLQEWQMKHIYSVDFYDRELTQPGIEYYNGICGKI | |
| GAM79 | NEHMNQFCQKNRINKNDFRMKKLHKQILCKKSSYYEIPFRFE | |
| Ref Seq. | SDQEVYDALNEFIKTMKKKEIIRRCVHLGQECDDYDLGKIYIS | |
| WP_119623382.1 | SNKYEQISNALYGSWDTIRKCIKEEYMDALPGKGEKKEEKAE | |
| | AAAKKEEYRSIADIDKIISLYGSEMDRTISAKKCITEICDMAGQ | |
| | ISIDPLVCNSDIKLLQNKEKTTEIKTILDSFLHVYQWGQTFIVSD | |
| | IIEKDSYFYSELEDVLEDFEGITTLYNHVRSYVTQKPYSTVKFK | |
| | LHFGSPTLANGWSQSKEYDNNAILLMRDQKFYLGIFNVRNKP | |
| | DKQIIKGHEKEEKGDYKKMIYNLLPGPSKMLPKVFITSRSGQE | |
| | TYKPSKHILDGYNEKRHIKSSPKFDLGYCWDLIDYYKECIHKH | |
| | PDWKNYDFHFSDTKDYEDISGFYREVEMQGYQIKWTYISADE | |
| | IQKLDEKGQIFLFQIYNKDFSVHSTGKDNLHTMYLKNLFSEEN | |
| | LKDIVLKLNGEAELFFRKASIKTPIVHKKGSVLVNRSYTQTVG | |
| | NKEIRVSIPEEYYTEIYNYLNHIGKGKLSSEAQRYLDEGKIKSF | |
| | TATKDIVKNYRYCCDHYFLHLPITINFKAKSDVAVNERTLAYI | |
| | AKKEDIHIIGIDRGERNLLYISVVDVHGNIREQRSFNIVNGYDY | |
| | QQKLKDREKSRDAARKNWEEIEKIKELKEGYLSMVIHYIAQL | |
| | VVKYNAVVAMEDLNYGFKTGRFKVERQVYQKFETMLIEKLH | |
| | YLVFKDREVCEEGGVLRGYQLTYIPESLKKVGKQCGFIFYVP | |
| | AGYTSKIDPTTGFVNLFSFKNLTNRESRQDFVGKFDEIRYDRD | |
| | KKMFEFSFDYNNYIKKGTILASTKWKVYTNGTRLKRIVVNGK | |
| | YTSQSMEVELTDAMEKMLQRAGIEYHDGKDLKGQIVEKGIE | |
| | AEIIDIFRLTVQMRNSRSESEDREYDRLISPVLNDKGEFFDTAT | |
| | ADKTLPQDADANGAYCIALKGLYEVKQIKENWKENEQFPRN | |
| | KLVQDNKTWFDFMQKKRYL | |
| PcCas12a - | MAKNFEDFKRLYSLSKTLRFEAKPIGATLDNIVKSGLLDEDEH | SEQ ID NO: 82 |
| reviously | RAASYVKVKKLIDEYHKVFIDRVLDDGCLPLENKGNNNSLAE | |
| known at | YYESYVSRAQDEDAKKKFKEIQQNLRSVIAKKLTEDKAYANL | |
| Cpf1 | FGNKLIESYKDKEDKKKIIDSDLIQFINTAESTQLDSMSQDEAK | |
| Prevotella | ELVKEFWGFVTYFYGFFDNRKNMYTAEEKSTGIAYRLVNEN | |
| copri | LPKFIDNIEAFNRAITRPEIQENMGVLYSDFSEYLNVESIQEMF | |
| Ref Seq. | QLDYYNMLLTQKQIDVYNAIIGGKTDDEHDVKIKGINEYINL | |
| WP_119227726.1 | YNQQHKDDKLPKLKALFKQILSDRNAISWLPEEFNSDQEVLN | |
| | AIKDCYERLAENVLGDKVLKSLLGSLADYSLDGIFIRNDLQLT | |
| | DISQKMFGNWGVIQNAIMQNIKRVAPARKHKESEEDYEKRIA | |
| | GIFKKADSFSISYINDCLNEADPNNAYFVENYFATFGAVNTPT | |
| | MQRENLFALVQNAYTEVAALLHSDYPTVKHLAQDKANVSKI | |
| | KALLDAIKSLQHFVKPLLGKGDESDKDERFYGELASLWAELD | |
| | TVTPLYNMIRNYMTRKPYSQKKIKLNFENPQLLGGWDANKE | |
| | KDYATIILRRNGLYYLAIMDKDSRKLLGKAMPSDGECYEKM | |
| | VYKFFKDVTTMIPKCSTQLKDVQAYFKVNTDDYVLNSKAFN | |
| | KPLTITKEVFDLNNVLYGKYKKFQKGYLTATGDNVGYTHAV | |
| | NVWIKFCMDFLNSYDSTCIYDFSSLKPESYLSLDAFYQDANLL | |
| | LYKLSFARASVSYINQLVEEGKMYLFQIYNKDFSEYSKGTPN | |
| | MHTLYWKALFDERNLADVVYKLNGQAEMFYRKKSIENTHPT | |
| | HPANHPILNKNKDNKKKESLFDYDLIKDRRYTVDKFMFHVPI | |
| | TMNFKSVGSENINQDVKAYLRHADDMHIIGIDRGERHLLYLV | |
| | VIDLQGNIKEQYSLNEIVNEYNGNTYHTNYHDLLDVREEERL | |
| | KARQSWQTIENIKELKEGYLSQVIHKITQLMVRYHAIVVLEDL | |
| | SKGFMRSRQKVEKQVYQKFEKMLIDKLNYLVDKKTDVSTPG | |
| | GLLNAYQLTCKSDSSQKLGKQSGFLFYIPAWNTSKIDPVTGFV | |
| | NLLDTHSLNSKEKIKAFFSKFDAIRYNKDKKWFEFNLDYDKF | |
| | GKKAEDTRTKWTLCTRGMRIDTFRNKEKNSQWDNQEVDLTT | |
| | EMKSLLEHYYIDIHGNLKDAISAQTDKAFFTGLLHILKLTLQM | |
| | RNSITGTETDYLVSPVADENGIFYDSRSCGNQLPENADANGA | |
| | YNIARKGLMLIEQIKNAEDLNNVKFDISNKAWLNFAQQKPYK | |
| | NG | |
| ErCas12a - | MFSAKLISDILPEFVIHNNNYSASEKEEKTQVIKLFSRFATSFK | SEQ ID NO: 83 |
| previously | DYFKNRANCFSANDISSSSCHRIVNDNAEIFFSNALVYRRIVK | |
| known at | NLSNDDINKISGDMKDSLKEMSLEEIYSYEKYGEFITQEGISFY | |
| Cpf1 | NDICGKVNLFMNLYCQKNKENKNLYKLRKLHKQILCIADTSY | |
| Eubacterium | EVPYKFESDEEVYQSVNGFLDNISSKHIVERLRKIGENYNGYN | |
| rectale | LDKIYIVSKFYESVSQKTYRDWETINTALEIHYNNILPGNGKS | |
| Ref Seq. | KADKVKKAVKNDLQKSITEINELVSNYKLCPDDNIKAETYIHE | |
| WP_119223642.1 | ISHILNNFEAQELKYNPEIHLVESELKASELKNVLDVIMNAFH | |
| | WCSVFMTEELVDKDNNFYAELEEIYDEIYPVISLYNLVRNYV | |
| | TQKPYSTKKIKLNFGIPTLADGWSKSKEYSNNAIILMRDNLYY | |
| | LGIFNAKNKPDKKIIEGNTSENKGDYKKMIYNLLPGPNKMIPK | |
| | VFLSSKTGVETYKPSAYILEGYKQNKHLKSSKDFDITFCHDLI | |
| | DYFKNCIAIHPEWKNFGFDFSDTSTYEDISGFYREVELQGYKI | |
| | DWTYISEKDIDLLQEKGQLYLFQIYNKDFSKKSSGNDNLHTM | |
| | YLKNLFSEENLKDIVLKLNGEAEIFFRKSSIKNPIIHKKGSILVN | |
| | RTYEAEEKDQFGNIQIVRKTIPENIYQELYKYFNDKSDKELSD | |
| | EAAKLKNVVGHHEAATNIVKDYRYTYDKYFLHMPITINFKA | |
| | NKTSFINDRILQYIAKEKDLHVIGIDRGERNLIYVSVIDTCGNIV | |
| | EQKSFNIVNGYDYQIKLKQQEGARQIARKEWKEIGKIKEIKEG | |
| | YLSLVIHEISKMVIKYNAIIAMEDLSYGFKKGRFKVERQVYQK | |
| | FETMLINKLNYLVFKDISITENGGLLKGYQLTYIPDKLKNVGH | |
| | QCGCIFYVPAAYTSKIDPTTGFVNIFKFKDLTVDAKREFIKKFD | |
| | SIRYDSDKNLFCFTFDYNNFITQNTVMSKSSWSVYTYGVRIKR | |
| | RFVNGRFSNESDTIDITKDMEKTLEMTDINWRDGHDLRQDIID | |
| | YEIVQHIFEIFKLTVQMRNSLSELEDRDYDRLISPVLNENNIFY | |
| | DSAKAGDALPKDADANGAYCIALKGLYEIKQITENWKEDGK | |
| | FSRDKLKISNKDWFDFIQNKRYL | |
| CsCas12a | MNYKTGLEDFIGKESLSKTLRNALIPTESTKIHMEEMGVIRDD | SEQ ID NO: 84 |
| previously | ELRAEKQQELKEIMDDYYRAFIEEKLGQIQGIQWNSLFQKME | |
| known at | ETMEDISVRKDLDKIQNEKRKEICCYFTSDKRFKDLFNAKLIT | |
| Cpf1 | DILPNFIKDNKEYTEEEKAEKEQTRVLFQRFATAFTNYFNQRR | |
| Clostridium | NNFSEDNISTAISFRIVNENSEIHLQNMRAFQRIEQQYPEEVCG | |
| sp. | MEEEYKDMLQEWQMKHIYLVDFYDRVLTQPGIEYYNGICGK | |
| AF34- | INEHMNQFCQKNRINKNDFRMKKLHKQILCKKSSYYEIPFRFE | |
| 10BH | SDQEVYDALNEFIKTMKEKEIICRCVHLGQKCDDYDLGKIYIS | |
| Ref Seq. | SNKYEQISNALYGSWDTIRKCIKEEYMDALPGKGEKKEEKAE | |
| WP_118538418.1 | AAAKKEEYRSIADIDKIISLYGSEMDRTISAKKCITEICDMAGQ | |
| | ISTDPLVCNSDIKLLQNKEKTTEIKTILDSFLHVYQWGQTFIVS | |
| | DIIEKDSYFYSELEDVLEDFEGITTLYNHVRSYVTQKPYSTVKF | |
| | KLHFGSPTLANGWSQSKEYDNNAILLMRDQKFYLGIFNVRNK | |
| | PDKQIIKGHEKEEKGDYKKMIYNLLPGPSKMLPKVFITSRSGQ | |
| | ETYKPSKHILDGYNEKRHIKSSPKFDLGYCWDLIDYYKECIHK | |
| | HPDWKNYDFHFSDTKDYEDISGFYREVEMQGYQIKWTYISA | |
| | DEIQKLDEKGQIFLFQIYNKDFSVHSTGKDNLHTMYLKNLFSE | |
| | ENLKDIVLKLNGEAELFFRKASIKTPVVHKKGSVLVNRSYTQT | |
| | VGDKEIRVSIPEEYYTEIYNYLNHIGRGKLSTEAQRYLEERKIK | |
| | SFTATKDIVKNYRYCCDHYFLHLPITINFKAKSDIAVNERTLA | |
| | YIAKKEDIHIIGIDRGERNLLYISVVDVHGNIREQRSFNIVNGY | |
| | DYQQKLKDREKSRDAARKNWEEIEKIKELKEGYLSMVIHYIA | |
| | QLVVKYNAVVAMEDLNYGFKTGRFKVERQVYQKFETMLIEK | |
| | LHYLVFKDREVCEEGGVLRGYQLTYIPESLKKVGKQCGFIFY | |
| | VPAGYTSKIDPTTGFVNLFSFKNLTNRESRQDFVGKFDEIRYD | |
| | RDKKMFEFSFDYNNYIKKGTMLASTKWKVYTNGTRLKRIVV | |
| | NGKYTSQSMEVELTDAMEKMLQRAGIEYHDGKDLKGQIVEK | |
| | GIEAEIIDIFRLTVQMRNSRSESEDREYDRLISPVLNDKGEFFDT | |
| | ATADKTLPQDADANGAYCIALKGLYEVKQIKENWKENEQFP | |
| | RNKLVQDNKTWFDFMQKKRYL | |
| BhCas12b | MATRSFILKIEPNEEVKKGLWKTHEVLNHGIAYYMNILKLIRQ | SEQ ID NO: 78 |
| Bacillus | EAIYEHHEQDPKNPKKVSKAEIQAELWDFVLKMQKCNSFTHE | |
| hisashii | VDKDEVFNILRELYEELVPSSVEKKGEANQLSNKFLYPLVDPN | |
| Ref Seq. | SQSGKGTASSGRKPRWYNLKIAGDPSWEEEKKKWEEDKKKD | |
| WP_095142515.1 | PLAKILGKLAEYGLIPLFIPYTDSNEPIVKEIKWMEKSRNQSVR | |
| | RLDKDMFIQALERFLSWESWNLKVKEEYEKVEKEYKTLEERI | |
| | KEDIQALKALEQYEKERQEQLLRDTLNTNEYRLSKRGLRGW | |
| | REIIQKWLKMDENEPSEKYLEVFKDYQRKHPREAGDYSVYEF | |
| | LSKKENHFIWRNHPEYPYLYATFCEIDKKKKDAKQQATFTLA | |
| | DPINHPLWVRFEERSGSNLNKYRILTEQLHTEKLKKKLTVQLD | |
| | RLIYPTESGGWEEKGKVDIVLLPSRQFYNQIFLDIEEKGKHAFT | |
| | YKDESIKFPLKGTLGGARVQFDRDHLRRYPHKVESGNVGRIY | |
| | FNMTVNIEPTESPVSKSLKIHRDDFPKVVNFKPKELTEWIKDS | |
| | KGKKLKSGIESLEIGLRVMSIDLGQRQAAAASIFEVVDQKPDI | |
| | EGKLFFPIKGTELYAVHRASFNIKLPGETLVKSREVLRKARED | |
| | NLKLMNQKLNFLRNVLHFQQFEDITEREKRVTKWISRQENSD | |
| | VPLVYQDELIQIRELMYKPYKDWVAFLKQLHKRLEVEIGKEV | |
| | KHWRKSLSDGRKGLYGISLKNIDEIDRTRKFLLRWSLRPTEPG | |
| | EVRRLEPGQRFAIDQLNHLNALKEDRLKKMANTIIMHALGYC | |
| | YDVRKKKWQAKNPACQIILFEDLSNYNPYEERSRFENSKLMK | |
| | WSRREIPRQVALQGEIYGLQVGEVGAQFSSRFHAKTGSPGIRC | |
| | SVVTKEKLQDNRFFKNLQREGRLTLDKIAVLKEGDLYPDKGG | |
| | EKFISLSKDRKCVTTHADINAAQNLQKRFWTRTHGFYKVYCK | |
| | AYQVDGQTVYIPESKDQKQKIIEEFGEGYFILKDGVYEWVNA | |
| | GKLKIKKGSSKQSSSELVDSDILKDSFDLASELKGEKLMLYRD | |
| | PSGNVFPSDKWMAAGVFFGKLERILISKLTNQYSISTIEDDSSK | |
| | QSM | |
| ThCas12b | MSEKTTQRAYTLRLNRASGECAVCQNNSCDCWHDALWATH | SEQ ID NO: 85 |
| Thermomonas | KAVNRGAKAFGDWLLTLRGGLCHTLVEMEVPAKGNNPPQRP | |
| hydrothermalis | TDQERRDRRVLLALSWLSVEDEHGAPKEFIVATGRDSADDRA | |
| Ref Seq. | KKVEEKLREILEKRDFQEHEIDAWLQDCGPSLKAHIREDAVW | |
| WP_072754838 | VNRRALFDAAVERIKTLTWEEAWDFLEPFFGTQYFAGIGDGK | |
| | DKDDAEGPARQGEKAKDLVQKAGQWLSARFGIGTGADFMS | |
| | MAEAYEKIAKWASQAQNGDNGKATIEKLACALRPSEPPTLDT | |
| | VLKCISGPGHKSATREYLKTLDKKSTVTQEDLNQLRKLADED | |
| | ARNCRKKVGKKGKKPWADEVLKDVENSCELTYLQDNSPAR | |
| | HREFSVMLDHAARRVSMAHSWIKKAEQRRRQFESDAQKLKN | |
| | LQERAPSAVEWLDRFCESRSMTTGANTGSGYRIRKRAIEGWS | |
| | YVVQAWAEASCDTEDKRIAAARKVQADPEIEKFGDIQLFEAL | |
| | AADEAICVWRDQEGTQNPSILIDYVTGKTAEHNQKRFKVPAY | |
| | RHPDELRHPVFCDFGNSRWSIQFAIHKEIRDRDKGAKQDTRQ | |
| | LQNRHGLKMRLWNGRSMTDVNLHWSSKRLTADLALDQNPN | |
| | PNPTEVTRADRLGRAASSAFDHVKIKNVFNEKEWNGRLQAPR | |
| | AELDRIAKLEEQGKTEQAEKLRKRLRWYVSFSPCLSPSGPFIV | |
| | YAGQHNIQPKRSGQYAPHAQANKGRARLAQLILSRLPDLRILS | |
| | VDLGHRFAAACAVWETLSSDAFRREIQGLNVLAGGSGEGDLF | |
| | LHVEMTGDDGKRRTVVYRRIGPDQLLDNTPHPAPWARLDRQ | |
| | FLIKLQGEDEGVREASNEELWTVHKLEVEVGRTVPLIDRMVR | |
| | SGFGKTEKQKERLKKLRELGWISAMPNEPSAETDEKEGEIRSI | |
| | SRSVDELMSSALGTLRLALKRHGNRARIAFAMTADYKPMPG | |
| | GQKYYFHEAKEASKNDDETKRRDNQIEFLQDALSLWHDLFSS | |
| | PDWEDNEAKKLWQNHIATLPNYQTPEEISAELKRVERNKKRK | |
| | ENRDKLRTAAKALAENDQLRQHLHDTWKERWESDDQQWKE | |
| | RLRSLKDWIFPRGKAEDNPSIRHVGGLSITRINTISGLYQILKAF | |
| | KMRPEPDDLRKNIPQKGDDELENFNRRLLEARDRLREQRVKQ | |
| | LASRIIEAALGVGRIKIPKNGKLPKRPRTTVDTPCHAVVIESLK | |
| | TYRPDDLRTRRENRQLMQWSSAKVRKYLKEGCELYGLHFLE | |
| | VPANYTSRQCSRTGLPGIRCDDVPTGDFLKAPWWRRAINTAR | |
| | EKNGGDAKDRFLVDLYDHLNNLQSKGEALPATVRVPRQGGN | |
| | LFIAGAQLDDTNKERRAIQADLNAAANIGLRALLDPDWRGR | |
| | WWYVPCKDGTSEPALDRIEGSTAFNDVRSLPTGDNSSRRAPR | |
| | EIENLWRDPSGDSLESGTWSPTRAYWDTVQSRVIELLRRHAG | |
| | LPTS | |
| LsCas12b | MSIRSFKLKLKTKSGVNAEQLRRGLWRTHQLINDGIAYYMN | SEQ ID NO: 86 |
| Laceyella | WLVLLRQEDLFIRNKETNEIEKRSKEEIQAVLLERVHKQQQRN | |
| sacchari | QWSGEVDEQTLLQALRQLYEEIVPSVIGKSGNASLKARFFLGP | |
| WP_132221894.1 | LVDPNNKTTKDVSKSGPTPKWKKMKDAGDPNWVQEYEKY | |
| | MAERQTLVRLEEMGLIPLFPMYTDEVGDIHWLPQASGYTRT | |
| | WDRDMFQQAIERLLSWESWNRRVRERRAQFEKKTHDFASRF | |
| | SESDVQWMNKLREYEAQQEKSLEENAFAPNEPYALTKKALR | |
| | GWERVYHSWMRLDSAASEEAYWQEVATCQTAMRGEFGDPA | |
| | IYQFLAQKENHDIWRGYPERVIDFAELNHLQRELRRAKEDAT | |
| | FTLPDSVDHPLWVRYEAPGGTNIHGYDLVQDTKRNLTLILDK | |
| | FILPDENGSWHEVKKVPFSLAKSKQFHRQVWLQEEQKQKKR | |
| | EVVFYDYSTNLPHLGTLAGAKLQWDRNFLNKRTQQQIEETGE | |
| | IGKVFFNISVDVRPAVEVKNGRLQNGLGKALTVLTHPDGTKI | |
| | VTGWKAEQLEKWVGESGRVSSLGLDSLSEGLRVMSIDLGQR | |
| | TSATVSVFEITKEAPDNPYKFFYQLEGTEMFAVHQRSFLLALP | |
| | GENPPQKIKQMREIRWKERNRIKQQVDQLSAILRLHKKVNED | |
| | ERIQAIDKLLQKVASWQLNEEIATAWNQALSQLYSKAKENDL | |
| | QWNQAIKNAHHQLEPVVGKQISLWRKDLSTGRQGIAGLSLW | |
| | SIEELEATKKLLTRWSKRSREPGVVKRIERFETFAKQIQHHINQ | |
| | VKENRLKQLANLIVMTALGYKYDQEQKKWIEVYPACQVVLF | |
| | ENLRSYRFSFERSRRENKKLMEWSHRSIPKLVQMQGELFGLQ | |
| | VADVYAAYSSRYHGRTGAPGIRCHALTEADLRNETNIIHELIE | |
| | AGFIKEEHRPYLQQGDLVPWSGGELFATLQKPYDNPRILTLH | |
| | ADINAAQNIQKRFWHPSMWFRVNCESVMEGEIVTYVPKNKT | |
| | VHKKQGKTFRFVKVEGSDVYEWAKWSKNRNKNTFSSITERK | |
| | PPSSMILFRDPSGTFFKEQEWVEQKTFWGKVQSMIQAYMKKT | |
| | IVQRMEE | |
| DtCas12b | MVLGRKDDTAELRRALWTTHEHVNLAVAEVERVLLRCRGRS | SEQ ID NO: 87 |
| Dsulfonatronum | YWTLDRRGDPVHVPESQVAEDALAMAREAQRRNGWPVVGE | |
| thiodismutans | DEEILLALRYLYEQIVPSCLLDDLGKPLKGDAQKIGTNYAGPL | |
| WP_031386437 | FDSDTCRRDEGKDVACCGPFHEVAGKYLGALPEWATPISKQE | |
| | FDGKDASHLRFKATGGDDAFFRVSIEKANAWYEDPANQDAL | |
| | KNKAYNKDDWKKEKDKGISSWAVKYIQKQLQLGQDPRTEV | |
| | RRKLWLELGLLPLFIPVFDKTMVGNLWNRLAVRLALAHLLS | |
| | WESWNHRAVQDQALARAKRDELAALFLGMEDGFAGLREYE | |
| | LRRNESIKQHAFEPVDRPYVVSGRALRSWTRVREEWLRHGDT | |
| | QESRKNICNRLQDRLRGKFGDPDVFHWLAEDGQEALWKERD | |
| | CVTSFSLLNDADGLLEKRKGYALMTFADARLHPRWAMYEAP | |
| | GGSNLRTYQIRKTENGLWADVVLLSPRNESAAVEEKTFNVRL | |
| | APSGQLSNVSFDQIQKGSKMVGRCRYQSANQQFEGLLGGAEI | |
| | LFDRKRIANEQHGATDLASKPGHVWFKLTLDVRPQAPQGWL | |
| | DGKGRPALPPEAKHFKTALSNKSKFADQVRPGLRVLSVDLGV | |
| | RSFAACSVFELVRGGPDQGTYFPAADGRTVDDPEKLWAKHE | |
| | RSFKITLPGENPSRKEEIARRAAMEELRSLNGDIRRLKAILRLS | |
| | VLQEDDPRTEHLRLFMEAIVDDPAKSALNAELFKGFGDDRFR | |
| | STPDLWKQHCHFFHDKAEKVVAERFSRWRTETRPKSSSWQD | |
| | WRERRGYAGGKSYWAVTYLEAVRGLILRWNMRGRTYGEVN | |
| | RQDKKQFGTVASALLHHINQLKEDRIKTGADMIIQAARGFVP | |
| | RKNGAGWVQVHEPCRLILFEDLARYRFRTDRSRRENSRLMR | |
| | WSHREIVNEVGMQGELYGLHVDTTEAGFSSRYLASSGAPGV | |
| | RCRHLVEEDFHDGLPGMHLVGELDWLLPKDKDRTANEARRL | |
| | LGGMVRPGMLVPWDGGELFATLNAASQLHVIHADINAAQNL | |
| | QRRFWGRCGEAIRIVCNQLSVDGSTRYEMAKAPKARLLGAL | |
| | QQLKNGDAPFHLTSIPNSQKPENSYVMTPTNAGKKYRAGPGE | |
| | KSSGEEDELALDIVEQAEELAQGRKTFFRDPSGVFFAPDRWLP | |
| | SEIYWSRIRRRIWQVTLERNSSGRQERAEMDEMPY | |

The prime editors described herein may also comprise Cas12a (Cpf1) (dCpf1) variants that may be used as a guide nucleotide sequence-programmable DNA-binding protein domain. The Cas12a (Cpf1) protein has a RuvC-like endonuclease domain that is similar to the RuvC domain of Cas9 but does not have a HNH endonuclease domain, and the N-terminal of Cas12a (Cpf1) does not have the alfa-helical recognition lobe of Cas9. It was shown in Zetsche et al., Cell, 163, 759-771, 2015 (which is incorporated herein by reference) that, the RuvC-like domain of Cas12a (Cpf1) is responsible for cleaving both DNA strands and inactivation of the RuvC-like domain inactivates Cas12a (Cpf1) nuclease activity.

In some embodiments, the napDNAbp is a single effector of a microbial CRISPR-Cas system. Single effectors of microbial CRISPR-Cas systems include, without limitation, Cas9, Cas12a (Cpf1), Cas12b1 (C2c1), Cas13a (C2c2), and Cas12c (C2c3). Typically, microbial CRISPR-Cas systems are divided into Class 1 and Class 2 systems. Class 1 systems have multisubunit effector complexes, while Class 2 systems have a single protein effector. For example, Cas9 and Cas12a (Cpf1) are Class 2 effectors. In addition to Cas9 and Cas12a (Cpf1), three distinct Class 2 CRISPR-Cas systems (Cas12b1, Cas13a, and Cas12c) have been described by Shmakov et al., “Discovery and Functional Characterization of Diverse Class 2 CRISPR Cas Systems”, Mol. Cell, 2015 Nov. 5; 60(3): 385-397, the entire contents of which are hereby incorporated by reference.

Effectors of two of the systems, Cas12b1 and Cas12c, contain RuvC-like endonuclease domains related to Cas12a. A third system, Cas13a contains an effector with two predicted HEPN RNase domains. Production of mature CRISPR RNA is tracrRNA-independent, unlike production of CRISPR RNA by Cas12b1. Cas12b1 depends on both CRISPR RNA and tracrRNA for DNA cleavage. Bacterial Cas13a has been shown to possess a unique RNase activity for CRISPR RNA maturation distinct from its RNA-activated single-stranded RNA degradation activity. These RNase functions are different from each other and from the CRISPR RNA-processing behavior of Cas12a. See, e.g., East-Seletsky, et al., “Two distinct RNase activities of CRISPR-Cas13a enable guide-RNA processing and RNA detection”, Nature, 2016 Oct. 13; 538(7624):270-273, the entire contents of which are hereby incorporated by reference. In vitro biochemical analysis of Cas13a in Leptotrichia shahii has shown that Cas13a is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris Cas12b1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a Cas13a protein. In some embodiments, the napDNAbp is a Cas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12b1 (C2c1), Cas13a (C2c2), or Cas12c (C2c3) protein. In some embodiments, the napDNAbp is a naturally-occurring Cas12b1 (C2c1), Cas13a (C2c2), or Cas12c (C2c3) protein.

### H. Cas9 Circular Permutants

In various embodiments, the prime editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure: N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB—Q99ZW2 (CAS9_STRP1) (numbering is based on the amino acid position in SEQ ID NO: 37)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circular permutants of other Cas9 proteins (including other Cas9 orthologs, variants, etc).

In particular embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 37):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In still other embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 37):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 88-97). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 37).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 37). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 37). In some embodiments, the C-terminal fragment that is rearranged to the N-terminus, includes or corresponds to the C-terminal 410 residues or less of a Cas9 (e.g., the Cas9 of SEQ ID NO: 37). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 37). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 37).

In other embodiments, circular permutant Cas9 variants may be defined as a topological rearrangement of a Cas9 primary structure based on the following method, which is based on S. pyogenes Cas9 of SEQ ID NO: 37: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 37) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-Cp¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 37, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 37, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 37 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLAN | SEQ ID NO: |
| | GEIRKRPLIETNGETGEIVWDKGRDFATVRKVLSMPQVNIVKKT | 88 |
| | EVQTGGFSKESILPKRNSDKLIARKKDWDPKKYGGFDSPTVAY | |
| | SVLVVAKVEKGKSKKLKSVKELLGITIMERSSFEKNPIDFLEAK | |
| | GYKEVKKDLIIKLPKYSLFELENGRKRMLASAGELQKGNELAL | |
| | PSKYVNFLYLASHYEKLKGSPEDNEQKQLFVEQHKHYLDEIIEQ | |
| | ISEFSKRVILADANLDKVLSAYNKHRDKPIREQAENIIHLFTLTN | |
| | LGAPAAFKYFDTTIDRKRYTSTKEVLDATLIHQSITGLYETRIDL | |
| | SQLGGDGGSGGSGGSGGSGGSGGSGGDKKYSIGLAIGTNSVG | |
| | WAVITDEYKVPSKKFKVLGNTDRHSIKKNLIGALLFDSGETAEA | |
| | TRLKRTARRRYTRRKNRICYLQEIFSNEMAKVDDSFFHRLEESF | |
| | LVEEDKKHERHPIFGNIVDEVAYHEKYPTIYHLRKKLVDSTDKA | |
| | DLRLIYLALAHMIKFRGHFLIEGDLNPDNSDVDKLFIQLVQTYN | |
| | QLFEENPINASGVDAKAILSARLSKSRRLENLIAQLPGEKKNGL | |
| | FGNLIALSLGLTPNFKSNFDLAEDAKLQLSKDTYDDDLDNLLA | |
| | QIGDQYADLFLAAKNLSDAILLSDILRVNTEITKAPLSASMIKRY | |
| | DEHHQDLTLLKALVRQQLPEKYKEIFFDQSKNGYAGYIDGGAS | |
| | QEEFYKFIKPILEKMDGTEELLVKLNREDLLRKQRTFDNGSIPH | |
| | QIHLGELHAILRRQEDFYPFLKDNREKIEKILTFRIPYYVGPLAR | |
| | GNSRFAWMTRKSEETITPWNFEEVVDKGASAQSFIERMTNFDK | |
| | NLPNEKVLPKHSLLYEYFTVYNELTKVKYVTEGMRKPAFLSGE | |
| | QKKAIVDLLFKTNRKVTVKQLKEDYFKKIECFDSVEISGVEDRF | |
| | NASLGTYHDLLKIIKDKDFLDNEENEDILEDIVLTLTLFEDREMI | |
| | EERLKTYAHLFDDKVMKQLKRRRYTGWGRLSRKLINGIRDKQ | |
| | SGKTILDFLKSDGFANRNFMQLIHDDSLTFKEDIQKAQVSGQGD | |
| | SLHEHIANLAGSPAIKKGILQTVKVVDELVKVMGRHKPENIVIE | |
| | MARENQTTQKGQKNSRERMKRIEEGIKELGSQILKEHPVENTQ | |
| | LQNEKLYLYYLQNGRDMYVDQELDINRLSDYDVDHIVPQSFLK | |
| | DDSIDNKVLTRSDKNRGKSDNVPSEEVVKKMKNYWRQLLNA | |
| | KLITQRKFDNLTKAERGGLSELDKAGFIKRQLVETRQITKHVAQ | |
| | ILDSRMNTKYDENDKLIREVKVITLKSKLVSDFRKDFQFYKVRE | |
| | INNYHHAHDAYLNAVVGTALIKKYPKLESEFVYG | |
| CP1028 | EIGKATAKYFFYSNIMNFFKTEITLANGEIRKRPLIETNGETGEIV | SEQ ID NO: |
| | WDKGRDFATVRKVLSMPQVNIVKKTEVQTGGFSKESILPKRNS | 89 |
| | DKLIARKKDWDPKKYGGFDSPTVAYSVLVVAKVEKGKSKKL | |
| | KSVKELLGITIMERSSFEKNPIDFLEAKGYKEVKKDLIIKLPKYS | |
| | LFELENGRKRMLASAGELQKGNELALPSKYVNFLYLASHYEK | |
| | LKGSPEDNEQKQLFVEQHKHYLDEIIEQISEFSKRVILADANLD | |
| | KVLSAYNKHRDKPIREQAENIIHLFTLTNLGAPAAFKYFDTTID | |
| | RKRYTSTKEVLDATLIHQSITGLYETRIDLSQLGGDGGSGGSGG | |
| | SGGSGGSGGSGGMDKKYSIGLAIGTNSVGWAVITDEYKVPSK | |
| | KFKVLGNTDRHSIKKNLIGALLFDSGETAEATRLKRTARRRYT | |
| | RRKNRICYLQEIFSNEMAKVDDSFFHRLEESFLVEEDKKHERHP | |
| | IFGNIVDEVAYHEKYPTIYHLRKKLVDSTDKADLRLIYLALAH | |
| | MIKFRGHFLIEGDLNPDNSDVDKLFIQLVQTYNQLFEENPINAS | |
| | GVDAKAILSARLSKSRRLENLIAQLPGEKKNGLFGNLIALSLGL | |
| | TPNFKSNFDLAEDAKLQLSKDTYDDDLDNLLAQIGDQYADLFL | |
| | AAKNLSDAILLSDILRVNTEITKAPLSASMIKRYDEHHQDLTLL | |
| | KALVRQQLPEKYKEIFFDQSKNGYAGYIDGGASQEEFYKFIKPI | |
| | LEKMDGTEELLVKLNREDLLRKQRTFDNGSIPHQIHLGELHAIL | |
| | RRQEDFYPFLKDNREKIEKILTFRIPYYVGPLARGNSRFAWMTR | |
| | KSEETITPWNFEEVVDKGASAQSFIERMTNFDKNLPNEKVLPK | |
| | HSLLYEYFTVYNELTKVKYVTEGMRKPAFLSGEQKKAIVDLLF | |
| | KTNRKVTVKQLKEDYFKKIECFDSVEISGVEDRFNASLGTYHD | |
| | LLKIIKDKDFLDNEENEDILEDIVLTLTLFEDREMIEERLKTYAH | |
| | LFDDKVMKQLKRRRYTGWGRLSRKLINGIRDKQSGKTILDFLK | |
| | SDGFANRNFMQLIHDDSLTFKEDIQKAQVSGQGDSLHEHIANL | |
| | AGSPAIKKGILQTVKVVDELVKVMGRHKPENIVIEMARENQTT | |
| | QKGQKNSRERMKRIEEGIKELGSQILKEHPVENTQLQNEKLYL | |
| | YYLQNGRDMYVDQELDINRLSDYDVDHIVPQSFLKDDSIDNK | |
| | VLTRSDKNRGKSDNVPSEEVVKKMKNYWRQLLNAKLITQRKF | |
| | DNLTKAERGGLSELDKAGFIKRQLVETRQITKHVAQILDSRMN | |
| | TKYDENDKLIREVKVITLKSKLVSDFRKDFQFYKVREINNYHH | |
| | AHDAYLNAVVGTALIKKYPKLESEFVYGDYKVYDVRKMIAKS | |
| | EQ | |
| CP1041 | NIMNFFKTEITLANGEIRKRPLIETNGETGEIVWDKGRDFATVR | SEQ ID NO: |
| | KVLSMPQVNIVKKTEVQTGGFSKESILPKRNSDKLIARKKDWD | 90 |
| | PKKYGGFDSPTVAYSVLVVAKVEKGKSKKLKSVKELLGITIME | |
| | RSSFEKNPIDFLEAKGYKEVKKDLIIKLPKYSLFELENGRKRML | |
| | ASAGELQKGNELALPSKYVNFLYLASHYEKLKGSPEDNEQKQ | |
| | LFVEQHKHYLDEIIEQISEFSKRVILADANLDKVLSAYNKHRDK | |
| | PIREQAENIIHLFTLTNLGAPAAFKYFDTTIDRKRYTSTKEVLDA | |
| | TLIHQSITGLYETRIDLSQLGGDGGSGGSGGSGGSGGSGGSGGD | |
| | KKYSIGLAIGTNSVGWAVITDEYKVPSKKFKVLGNTDRHSIKK | |
| | NLIGALLFDSGETAEATRLKRTARRRYTRRKNRICYLQEIFSNE | |
| | MAKVDDSFFHRLEESFLVEEDKKHERHPIFGNIVDEVAYHEKY | |
| | PTIYHLRKKLVDSTDKADLRLIYLALAHMIKFRGHFLIEGDLNP | |
| | DNSDVDKLFIQLVQTYNQLFEENPINASGVDAKAILSARLSKSR | |
| | RLENLIAQLPGEKKNGLFGNLIALSLGLTPNFKSNFDLAEDAKL | |
| | QLSKDTYDDDLDNLLAQIGDQYADLFLAAKNLSDAILLSDILR | |
| | VNTEITKAPLSASMIKRYDEHHQDLTLLKALVRQQLPEKYKEIF | |
| | FDQSKNGYAGYIDGGASQEEFYKFIKPILEKMDGTEELLVKLN | |
| | REDLLRKQRTFDNGSIPHQIHLGELHAILRRQEDFYPFLKDNRE | |
| | KIEKILTFRIPYYVGPLARGNSRFAWMTRKSEETITPWNFEEVV | |
| | DKGASAQSFIERMTNFDKNLPNEKVLPKHSLLYEYFTVYNELT | |
| | KVKYVTEGMRKPAFLSGEQKKAIVDLLFKTNRKVTVKQLKED | |
| | YFKKIECFDSVEISGVEDRFNASLGTYHDLLKIIKDKDFLDNEE | |
| | NEDILEDIVLTLTLFEDREMIEERLKTYAHLFDDKVMKQLKRR | |
| | RYTGWGRLSRKLINGIRDKQSGKTILDFLKSDGFANRNFMQLI | |
| | HDDSLTFKEDIQKAQVSGQGDSLHEHIANLAGSPAIKKGILQTV | |
| | KVVDELVKVMGRHKPENIVIEMARENQTTQKGQKNSRERMK | |
| | RIEEGIKELGSQILKEHPVENTQLQNEKLYLYYLQNGRDMYVD | |
| | QELDINRLSDYDVDHIVPQSFLKDDSIDNKVLTRSDKNRGKSD | |
| | NVPSEEVVKKMKNYWRQLLNAKLITQRKFDNLTKAERGGLSE | |
| | LDKAGFIKRQLVETRQITKHVAQILDSRMNTKYDENDKLIREV | |
| | KVITLKSKLVSDFRKDFQFYKVREINNYHHAHDAYLNAVVGT | |
| | ALIKKYPKLESEFVYGDYKVYDVRKMIAKSEQEIGKATAKYFF | |
| | YS | |
| CP1249 | PEDNEQKQLFVEQHKHYLDEIIEQISEFSKRVILADANLDKVLS | SEQ ID NO: |
| | AYNKHRDKPIREQAENIIHLFTLTNLGAPAAFKYFDTTIDRKRY | 91 |
| | TSTKEVLDATLIHQSITGLYETRIDLSQLGGDGGSGGSGGSGGS | |
| | GGSGGSGGMDKKYSIGLAIGTNSVGWAVITDEYKVPSKKFKV | |
| | LGNTDRHSIKKNLIGALLFDSGETAEATRLKRTARRRYTRRKN | |
| | RICYLQEIFSNEMAKVDDSFFHRLEESFLVEEDKKHERHPIFGNI | |
| | VDEVAYHEKYPTIYHLRKKLVDSTDKADLRLIYLALAHMIKFR | |
| | GHFLIEGDLNPDNSDVDKLFIQLVQTYNQLFEENPINASGVDAK | |
| | AILSARLSKSRRLENLIAQLPGEKKNGLFGNLIALSLGLTPNFKS | |
| | NFDLAEDAKLQLSKDTYDDDLDNLLAQIGDQYADLFLAAKNL | |
| | SDAILLSDILRVNTEITKAPLSASMIKRYDEHHQDLTLLKALVR | |
| | QQLPEKYKEIFFDQSKNGYAGYIDGGASQEEFYKFIKPILEKMD | |
| | GTEELLVKLNREDLLRKQRTFDNGSIPHQIHLGELHAILRRQED | |
| | FYPFLKDNREKIEKILTFRIPYYVGPLARGNSRFAWMTRKSEETI | |
| | TPWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDDK | |
| | VMKQLKRRRYTGWGRLSRKLINGIRDKQSGKTILDFLKSDGFA | |
| | NRNFMQLIHDDSLTFKEDIQKAQVSGQGDSLHEHIANLAGSPAI | |
| | KKGILQTVKVVDELVKVMGRHKPENIVIEMARENQTTQKGQK | |
| | NSRERMKRIEEGIKELGSQILKEHPVENTQLQNEKLYLYYLQN | |
| | GRDMYVDQELDINRLSDYDVDHIVPQSFLKDDSIDNKVLTRSD | |
| | KNRGKSDNVPSEEVVKKMKNYWRQLLNAKLITQRKFDNLTK | |
| | AERGGLSELDKAGFIKRQLVETRQITKHVAQILDSRMNTKYDE | |
| | NDKLIREVKVITLKSKLVSDFRKDFQFYKVREINNYHHAHDAY | |
| | LNAVVGTALIKKYPKLESEFVYGDYKVYDVRKMIAKSEQEIGK | |
| | ATAKYFFYSNIMNFFKTEITLANGEIRKRPLIETNGETGEIVWDK | |
| | GRDFATVRKVLSMPQVNIVKKTEVQTGGFSKESILPKRNSDKLI | |
| | ARKKDWDPKKYGGFDSPTVAYSVLVVAKVEKGKSKKLKSVK | |
| | ELLGITIMERSSFEKNPIDFLEAKGYKEVKKDLIIKLPKYSLFELE | |
| | NGRKRMLASAGELQKGNELALPSKYVNFLYLASHYEKLKGS | |
| CP1300 | KPIREQAENIIHLFTLTNLGAPAAFKYFDTTIDRKRYTSTKEVLD | SEQ ID NO: |
| | ATLIHQSITGLYETRIDLSQLGGDGGSGGSGGSGGSGGSGGSGG | 92 |
| | DKKYSIGLAIGTNSVGWAVITDEYKVPSKKFKVLGNTDRHSIK | |
| | KNLIGALLFDSGETAEATRLKRTARRRYTRRKNRICYLQEIFSN | |
| | EMAKVDDSFFHRLEESFLVEEDKKHERHPIFGNIVDEVAYHEK | |
| | YPTIYHLRKKLVDSTDKADLRLIYLALAHMIKFRGHFLIEGDLN | |
| | PDNSDVDKLFIQLVQTYNQLFEENPINASGVDAKAILSARLSKS | |
| | RRLENLIAQLPGEKKNGLFGNLIALSLGLTPNFKSNFDLAEDAK | |
| | LQLSKDTYDDDLDNLLAQIGDQYADLFLAAKNLSDAILLSDIL | |
| | RVNTEITKAPLSASMIKRYDEHHQDLTLLKALVRQQLPEKYKEI | |
| | FFDQSKNGYAGYIDGGASQEEFYKFIKPILEKMDGTEELLVKLN | |
| | REDLLRKQRTFDNGSIPHQIHLGELHAILRRQEDFYPFLKDNRE | |
| | KIEKILTFRIPYYVGPLARGNSRFAWMTRKSEETITPWNFEEVV | |
| | DKGASAQSFIERMTNFDKNLPNEKVLPKHSLLYEYFTVYNELT | |
| | KVKYVTEGMRKPAFLSGEQKKAIVDLLFKTNRKVTVKQLKED | |
| | YFKKIECFDSVEISGVEDRFNASLGTYHDLLKIIKDKDFLDNEE | |
| | NEDILEDIVLTLTLFEDREMIEERLKTYAHLFDDKVMKQLKRR | |
| | RYTGWGRLSRKLINGIRDKQSGKTILDFLKSDGFANRNFMQLI | |
| | HDDSLTFKEDIQKAQVSGQGDSLHEHIANLAGSPAIKKGILQTV | |
| | KVVDELVKVMGRHKPENIVIEMARENQTTQKGQKNSRERMK | |
| | RIEEGIKELGSQILKEHPVENTQLQNEKLYLYYLQNGRDMYVD | |
| | QELDINRLSDYDVDHIVPQSFLKDDSIDNKVLTRSDKNRGKSD | |
| | NVPSEEVVKKMKNYWRQLLNAKLITQRKFDNLTKAERGGLSE | |
| | LDKAGFIKRQLVETRQITKHVAQILDSRMNTKYDENDKLIREV | |
| | KVITLKSKLVSDFRKDFQFYKVREINNYHHAHDAYLNAVVGT | |
| | ALIKKYPKLESEFVYGDYKVYDVRKMIAKSEQEIGKATAKYFF | |
| | YSNIMNFFKTEITLANGEIRKRPLIETNGETGEIVWDKGRDFAT | |
| | VRKVLSMPQVNIVKKTEVQTGGFSKESILPKRNSDKLIARKKD | |
| | WDPKKYGGFDSPTVAYSVLVVAKVEKGKSKKLKSVKELLGITI | |
| | MERSSFEKNPIDFLEAKGYKEVKKDLIIKLPKYSLFELENGRKR | |
| | MLASAGELQKGNELALPSKYVNFLYLASHYEKLKGSPEDNEQ | |
| | KQLFVEQHKHYLDEIIEQISEFSKRVILADANLDKVLSAYNKHR | |
| | D | |

The Cas9 circular permutants that may be useful in the prime editing constructs described herein. Exemplary C-terminal fragments of Cas9, based on the Cas9 of SEQ ID NO: 37, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 | DYKVYDVRKMIAKSEQEIGKATAKYFFYSNIMNFFKTEITLAN | SEQ ID NO: |
| C- | GEIRKRPLIETNGETGEIVWDKGRDFATVRKVLSMPQVNIVKK | 93 |
| terminal | TEVQTGGFSKESILPKRNSDKLIARKKDWDPKKYGGFDSPTVA | |
| fragment | YSVLVVAKVEKGKSKKLKSVKELLGITIMERSSFEKNPIDFLEA | |
| | KGYKEVKKDLIIKLPKYSLFELENGRKRMLASAGELQKGNELA | |
| | LPSKYVNFLYLASHYEKLKGSPEDNEQKQLFVEQHKHYLDEIIE | |
| | QISEFSKRVILADANLDKVLSAYNKHRDKPIREQAENIIHLFTLT | |
| | NLGAPAAFKYFDTTIDRKRYTSTKEVLDATLIHQSITGLYETRID | |
| | LSQLGGD | |
| CP1028 | EIGKATAKYFFYSNIMNFFKTEITLANGEIRKRPLIETNGETGEIV | SEQ ID NO: 94 |
| C- | WDKGRDFATVRKVLSMPQVNIVKKTEVQTGGFSKESILPKRNS | |
| terminal | DKLIARKKDWDPKKYGGFDSPTVAYSVLVVAKVEKGKSKKL | |
| fragment | KSVKELLGITIMERSSFEKNPIDFLEAKGYKEVKKDLIIKLPKYS | |
| | LFELENGRKRMLASAGELQKGNELALPSKYVNFLYLASHYEK | |
| | LKGSPEDNEQKQLFVEQHKHYLDEIIEQISEFSKRVILADANLD | |
| | KVLSAYNKHRDKPIREQAENIIHLFTLTNLGAPAAFKYFDTTID | |
| | RKRYTSTKEVLDATLIHQSITGLYETRIDLSQLGGD | |
| CP1041 | NIMNFFKTEITLANGEIRKRPLIETNGETGEIVWDKGRDFATVR | SEQ ID NO: |
| C- | KVLSMPQVNIVKKTEVQTGGFSKESILPKRNSDKLIARKKDWD | 95 |
| terminal | PKKYGGFDSPTVAYSVLVVAKVEKGKSKKLKSVKELLGITIME | |
| fragment | RSSFEKNPIDFLEAKGYKEVKKDLIIKLPKYSLFELENGRKRML | |
| | ASAGELQKGNELALPSKYVNFLYLASHYEKLKGSPEDNEQKQ | |
| | LFVEQHKHYLDEIIEQISEFSKRVILADANLDKVLSAYNKHRDK | |
| | PIREQAENIIHLFTLTNLGAPAAFKYFDTTIDRKRYTSTKEVLDA | |
| | TLIHQSITGLYETRIDLSQLGGD | |
| CP1249 | PEDNEQKQLFVEQHKHYLDEIIEQISEFSKRVILADANLDKVLS | SEQ ID NO: |
| C- | AYNKHRDKPIREQAENIIHLFTLTNLGAPAAFKYFDTTIDRKRY | 96 |
| terminal | TSTKEVLDATLIHQSITGLYETRIDLSQLGGD | |
| fragment | | |
| CP1300 | KPIREQAENIIHLFTLTNLGAPAAFKYFDTTIDRKRYTSTKEVLD | SEQ ID NO: |
| C- | ATLIHQSITGLYETRIDLSQLGGD | 97 |
| terminal | | |
| fragment | | |

I. Cas9 Variants with Modified PAM Specificities

The prime editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C, G, or T) at its 3Y-end. In some embodiments, the Cas9 protein exhibits activity on a target sequence comprising a 5′-NGG-3′ PAM sequence at its 3Y-end. In some embodiments, the Cas9 protein exhibits activity on a target sequence comprising a 5″-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the Cas9 protein comprises a combination of mutations that exhibit activity on a target sequence comprising a 5′-NAA-3′ PAM sequence at its 3′-end. In some embodiments, the combination of mutations are present in any one of the clones listed in Table 1. In some embodiments, the combination of mutations are conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: 37)

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, S409I, E427G, G715C, D1135N, E1219V, Q1221H, H1264Y, A1320V,
R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H,
H1264H, A1320V, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, V743I, R753G, E762G, D1135N, D1180G, K1211R, E1219V,
Q1221H, H1264Y, A1320V, R1333K
A10T, I322V, S409I, E427G, R753G, D1135N, D1180G, K1211R, E1219V, Q1221H,
H1264Y, S1274R, A1320V, R1333K
A10T, I322V, S409I, E427G, A589S, R753G, D1135N, E1219V, Q1221H, H1264H,
A1320V, R1333K
A10T, I322V, S409I, E427G, R753G, E757K, G865G, D1135N, E1219V, Q1221H, H1264Y,
A1320V, R1333K
A10T, I322V, S409I, E427G, R654L, R753G, E757K, D1135N, E1219V, Q1221H, H1264Y,
A1320V, R1333K
A10T, I322V, S409I, E427G, K599R, M631A, R654L, K673E, V743I, R753G, N758H,
E762G, D1135N, D1180G, E1219V, Q1221H, Q1256R, H1264Y, A1320V, A1323D,
R1333K
A10T, I322V, S409I, E427G, R654L, K673E, V743I, R753G, E762G, N869S, N1054D,
R1114G, D1135N, D1180G, E1219V, Q1221H, H1264Y, A1320V, A1323D, R1333K
A10T, I322V, S409I, E427G, R654L, L727I, V743I, R753G, E762G, R859S, N946D,
F1134L, D1135N, D1180G, E1219V, Q1221H, H1264Y, N1317T, A1320V, A1323D,
R1333K
A10T, I322V, S409I, E427G, R654L, K673E, V743I, R753G, E762G, N803S, N869S,
Y1016D, G1077D, R1114G, F1134L, D1135N, D1180G, E1219V, Q1221H, H1264Y,
V1290G, L1318S, A1320V, A1323D, R1333K
A10T, I322V, S409I, E427G, R654L, K673E, V743I, R753G, E762G, N803S, N869S,
Y1016D, G1077D, R1114G, F1134L, D1135N, K1151E, D1180G, E1219V, Q1221H,
H1264Y, V1290G, E1318S, A1320V, R1333K
A10T, I322V, S409I, E427G, R654L, K673E, V743I, R753G, E762G, N803S, N869S,
Y1016D, G1077D, R1114G, F1134L, D1135N, D1180G, E1219V, Q1221H, H1264Y,
V1290G, L1318S, A1320V, A1323D, R1333K
A10T, I322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, I322V, S409I, E427G, E630K, R654L, K673E, V743I, R753G, E762G, Q768H,
N803S, N869S, Y1016D, G1077D, R1114G, F1134L, D1135N, D1180G, E1219V, Q1221H,
H1264Y, L1318S, A1320V, R1333K
A10T, I322V, S409I, E427G, R654L, K673E, F693L, V743I, R753G, E762G, Q768H,
N803S, N869S, Y1016D, G1077D, R1114G, F1134L, D1135N, D1180G, E1219V, Q1221H,
G1223S, H1264Y, L1318S, A1320V, R1333K
A10T, I322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, R654L, K673E, V743I, R753G, E762G, M673I, N803S, N869S,
G1077D, R1114G, D1135N, V1139A, D1180G, E1219V, Q1221H, A1320V, R1333K
A10T, I322V, S409I, E427G, R654L, K673E, V743I, R753G, E762G, N803S, N869S,
R1114G, D1135N, E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 37. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 37 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 37 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3′ PAM sequence at its 3′-end. In some embodiments, the combination of mutations are present in any one of the clones listed in Table 2. In some embodiments, the combination of mutations are conservative mutations of the clones listed in Table 2. In some embodiments, the Cas9 protein comprises the combination of mutations of any one of the Cas9 clones listed in Table 2.

TABLE 2
NAC PAM Clones
MUTATIONS FROM WILD-TYPE SPCAS9 (E.G., SEQ ID NO: 37)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N,
R1335Q, T1337N
E627K, T638P, K652T, R753G, N803S, K959N, R1114G, D1135N, K1156E, E1219V,
D1332N, R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N, R1114G, D1135N, E1219V,
D1332G, R1335Q, T1337N
E627K, T638P, R753G, N803S, K959N, R1114G, D1135N, E1219V, N1266H, D1332N,
R1335Q, T1337N
E627K, T638P, R753G, N803S, K959N, I1057T, R1114G, D1135N, E1219V, D1332N,
R1335Q, T1337N
E627K, T638P, R753G, N803S, K959N, R1114G, D1135N, E1219V, D1332N, R1335Q,
T1337N
E627K, M631I, T638P, R753G, N803S, K959N, Y1036H, R1114G, D1135N, E1219V,
D1251G, D1332G, R1335Q, T1337N
E627K, T638P, R753G, N803S, V875I, K959N, Y1016C, R1114G, D1135N, E1219V,
D1251G, D1332G, R1335Q, T1337N, I1348V
K608R, E627K, T638P, V647I, R654L, R753G, N803S, T804A, K848N, V922A, K959N,
R1114G, D1135N, E1219V, D1332N, R1335Q, T1337N
K608R, E627K, T638P, V647I, R753G, N803S, V922A, K959N, K1014N, V1015A, R1114G,
D1135N, K1156N, E1219V, N1252D, D1332N, R1335Q, T1337N
K608R, E627K, R629G, T638P, V647I, A711T, R753G, K775R, K789E, N803S, K959N,
V1015A, Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N, Y1016S, R1114G, D1135N,
E1219V, N1286H, D1332N, R1335Q, T1337N
I670S, K608R, E627K, E630G, T638P, V647I, R653K, R753G, I795L, K797N, N803S,
K866R, K890N, K959N, Y1016C, R1114G, D1135N, E1219V, D1332N, R1335Q, T1337N
K608R, E627K, T638P, V647I, T740A, G752R, R753G, K797N, N803S, K948E, K959N,
V1015A, Y1016S, R1114G, D1135N, E1219V, N1266H, D1332N, R1335Q, T1337N
I570T, A589V, K608R, E627K, T638P, V647I, R654L, Q716R, R753G, N803S, K948E,
K959N, Y1016S, R1114G, D1135N, E1207G, E1219V, N1234D, D1332N, R1335Q, T1337N
K608R, E627K, R629G, T638P, V647I, R654L, Q740R, R753G, N803S, K959N, N990S,
T995S, V1015A, Y1036D, R1114G, D1135N, E1207G, E1219V, N1234D, N1266H, D1332N,
R1335Q, T1337N
I562F, V565D, I570T, K608R, L625S, E627K, T638P, V647I, R654I, G752R, R753G, N803S,
N808D, K959N, M1021L, R1114G, D1135N, N1177S, N1234D, D1332N, R1335Q, T1337N
I562F, I570T, K608R, E627K, T638P, V647I, R753G, E790A, N803S, K959N, V1015A,
Y1036H, R1114G, D1135N, D1180E, A1184T, E1219V, D1332N, R1335Q, T1337N
I570T, K608R, E627K, T638P, V647I, R654H, R753G, E790A, N803S, K959N, V1015A,
R1114G, D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R,
K890N, K959N, Y1016C, R1114G, D1135N, E1219V, D1332N, R1335Q, T1337N
I570T, K608R, E627K, T638P, V647I, R654H, R753G, E790A, N803S, K959N, V1016A,
R1114G, D1135N, E1219V, K1246E, D1332N, R1335Q, T1337N
K608R, E627K, T638P, V647I, R654L, K673E, R753G, E790A, N803S, K948E, K959N,
R1114G, D1127G, D1135N, D1180E, E1219V, N1286H, D1332N, R1335Q, T1337N
K608R, L625S, E627K, T638P, V647I, R654I, I670T, R753G, N803S, N808D, K959N,
M1021L, R1114G, D1135N, E1219V, N1286H, D1332N, R1335Q, T1337N
E627K, M631V, T638P, V647I, K710E, R753G, N803S, N808D, K948E, M1021L, R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 37. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 37 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 37 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combination of mutations are present in any one of the clones listed in Table 3. In some embodiments, the combination of mutations are conservative mutations of the clones listed in Table 3. In some embodiments, the Cas9 protein comprises the combination of mutations of any one of the Cas9 clones listed in Table 3.

TABLE 3
NAT PAM Clones
MUTATIONS FROM WILD-TYPE SPCAS9 (E.G., SEQ ID NO: 37)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K,
A1293T, P1321S, D1322G, R1335L, T1339I
F575S, M631L, R654L, V748I, V743I, R753G, D853E, V922A, R1114G D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S,
E1219V, Q1221H, P1249S, N1286K, P1321S, D1322G, R1335L
M631L, R654L, R753G, K797E, D853E, V922A, D1012A, R1114G D1135N, G1218S,
E1219V, Q1221H, P1249S, N1317K, P1321S, D1322G, R1335L
F575S, M631L, R654L, R664K, R753G, D853E, V922A, R1114G, Y1131C, D1135N,
D1180G, G1218S, E1219V, Q1221H, P1249S, P1321S, D1322G, R1335L
F575S, M631L, R654L, R664K, R753G, D853E, V922A, R1114G, Y1131C, D1135N,
D1180G, G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N,
D1180G, G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D1180G,
G1218S, E1219V, Q1221H, P1249S, P1321S, D1332G, R1335L
M631L, R654L, R664K, R753G, D853E, I1057V, Y1131C, D1135N, D1180G, G1218S,
E1219V, Q1221H, P1249S, P1321S, D1332G, R1335L
M631L, R654L, R664K, R753G, I1057V, R1114G, Y1131C, D1135N, D1180G, G1218S,
E1219V, Q1221H, P1249S, P1321S, D1332G, R1335L

The above description of various napDNAbps which can be used in connection with the presently disclose prime editors is not meant to be limiting in any way. The prime editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The prime editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR (SEQ ID NO: 98), which has the following amino acid sequence (with the V, R, Q, R substitutions relative to the SpCas9 (H840A) of SEQ ID NO: 68 being show in bold underline. In addition, the methionine residue in SpCas9 (H840) was removed for SpCas9 (H840A) VRQR)

(SEQ ID NO: 98)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD

In another particular embodiment, the Cas9 variant having expanded PAM capabilities is SpCas9 (H840A) VRER, which has the following amino acid sequence (with the V, R, E, R substitutions relative to the SpCas9 (H840A) of SEQ ID NO: 68 being shown in bold underline. In addition, the methionine residue in SpCas9 (H840) was removed for SpCas9 (H840A) VRER):

(SEQ ID NO: 99)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKEYRSTKEVLDATLIHQS
ITGLYETRIDLSQLGGD

In some embodiments, the napDNAbp that functions with a non-canonical PAM sequence is an Argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argonaute (MpAgo) protein. The CRISPR-associated Marinitoga piezophila Argonaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other Argonaute proteins may be used, and are within the scope of this disclosure.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (D917, E1006, and D1255) (SEQ ID NO: 100), which has the following amino acid sequence:

(SEQ ID NO: 100)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 55). In some embodiments, the nucleic acid programmable DNA binding protein (napDNAbp) is a nucleic acid programmable DNA binding protein that does not require a canonical (NGG) PAM sequence. In some embodiments, the napDNAbp is an Argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 101.

The disclosed fusion proteins may comprise a napDNAbp domain having at least 80%, at least 85%, at least 90%, at least 95%, or at least 99% sequence identity with wild type Natronobacterium gregoryi Argonaute (SEQ ID NO: 101), which has the following amino acid sequence:

(SEQ ID NO: 101)
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Any of the references noted above which relate to Cas9 or Cas9 equivalents are hereby incorporated by reference in their entireties, if not already stated so.

J. Divided napDNAbp Domains for Split PE Delivery

In various embodiments, the prime editors described herein may be delivered to cells as two or more fragments which become assembled inside the cell (either by passive assembly, or by active assembly, such as using split intein sequences) into a reconstituted prime editor. In some cases, the self assembly may be passive whereby the two or more prime editor fragments associate inside the cell covalently or non-covalently to reconstitute the prime editor. In other cases, the self-assembly may be catalyzed by dimerization domains installed on each of the fragments. Examples of dimerization domains are described herein. In still other cases, the self-assembly may be catalyzed by split intein sequences installed on each of the prime editor fragments.

Split PE delivery may be advantageous to address various size constraints of different delivery approaches. For example, delivery approaches may include virus-based delivery methods, messenger RNA-based delivery methods, or RNP-based delivery (ribonucleoprotein-based delivery). And, each of these methods of delivery may be more efficient and/or effective by dividing up the prime editor into smaller pieces. Once inside the cell, the smaller pieces can assemble into a functional prime editor. Depending on the means of splitting, the divided prime editor fragments can be reassembled in a non-covalent manner or a covalent manner to reform the prime editor. In one embodiment, the prime editor can be split at one or more split sites into two or more fragments. The fragments can be unmodified (other than being split). Once the fragments are delivered to the cell (e.g., by direct delivery of a ribonucleoprotein complex or by nucleic delivery—e.g., mRNA delivery or virus vector based delivery), the fragments can reassociate covalently or non-covalently to reconstitute the prime editor. In another embodiment, the prime editor can be split at one or more split sites into two or more fragments. Each of the fragments can be modified to comprise a dimerization domain, whereby each fragment that is formed is coupled to a dimerization domain. Once delivered or expressed within a cell, the dimerization domains of the different fragments associate and bind to one another, bringing the different prime editor fragments together to reform a functional prime editor. In yet another embodiment, the prime editor fragment may be modified to comprise a split intein. Once delivered or expressed within a cell, the split intein domains of the different fragments associate and bind to one another, and then undergo trans-splicing, which results in the excision of the split-intein domains from each of the fragments, and a concomitant formation of a peptide bond between the fragments, thereby restoring the prime editor.

In one embodiment, the prime editor can be delivered using a split-intein approach.

The location of the split site can be positioned between any one or more pair of residues in the prime editor and in any domains therein, including within the napDNAbp domain, the polymerase domain (e.g., RT domain), linker domain that joins the napDNAbp domain and the polymerase domain.

In one embodiment, depicted in FIG. 66, the prime editor (PE) is divided at a split site within the napDNAbp.

In certain embodiments, the napDNAbp is a canonical SpCas9 polypeptide of SEQ ID NO: 37. In certain embodiments, the SpCas9 is split into two fragments at a split site located between residues 1 and 2, or 2 and 3, or 3 and 4, or 4 and 5, or 5 and 6, or 6 and 7, or 7 and 8, or 8 and 9, or 9 and 10, or between any two pair of residues located anywhere between residues 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 1000-1100, 1100-1200, 1200-1300, or 1300-1368 of canonical SpCas9 of SEQ ID NO: 37.

In certain embodiments, a napDNAbp is split into two fragments at a split site that is located at a pair of residue that corresponds to any two pair of residues located anywhere between positions 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 1000-1100, 1100-1200, 1200-1300, or 1300-1368 of canonical SpCas9 of SEQ ID NO: 37.

In certain embodiments, the SpCas9 is split into two fragments at a split site located between residues 1 and 2, or 2 and 3, or 3 and 4, or 4 and 5, or 5 and 6, or 6 and 7, or 7 and 8, or 8 and 9, or 9 and 10, or between any two pair of residues located anywhere between residues 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 1000-1100, 1100-1200, 1200-1300, or 1300-1368 of canonical SpCas9 of SEQ ID NO: 37. In certain embodiments, the split site is located one or more polypeptide bond sites (i.e., a “split site or split-intein split site”), fused to a split intein, and then delivered to cells as separately-encoded fusion proteins. Once the split-intein fusion proteins (i.e., protein halves) are expressed within a cell, the proteins undergo trans-splicing to form a complete or whole PE with the concomitant removal of the joined split-intein sequences.

For example, as shown in FIG. 66, the N-terminal extein can be fused to a first split-intein (e.g., N intein) and the C-terminal extein can be fused to a second split-intein (e.g., C intein). The N-terminal extein becomes fused to the C-terminal extein to reform a whole prime editor comprising an napDNAbp domain and a polymerase domain (e.g., RT domain) upon the self-association of the N intein and the C intein inside the cell, followed by their self-excision, and the concomitant formation of a peptide bond between the N-terminal extein and C-terminal extein portions of a whole prime editor (PE).

To take advantage of a split-PE delivery strategy using split-inteins, the prime editor needs to be divided at one or more split sites to create at least two separate halves of a prime editor, each of which may be rejoined inside a cell if each half is fused to a split-intein sequence.

In certain embodiments, the prime editor is split at a single split site. In certain other embodiments, the prime editor is split at two split sites, or three split sites, or four split sites, or more.

In a preferred embodiment, the prime editor is split at a single split site to create two separate halves of a prime editor, each of which can be fused to a split intein sequence

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostoc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product, e.g., as shown in FIGS. 66 and 67 with regard to the formation of a complete Prime editor from two separately-expressed halves.

In various embodiments described herein, the continuous evolution methods (e.g., PACE) may be used to evolve a first portion of a base editor. A first portion could include a single component or domain, e.g., a Cas9 domain, a deaminase domain, or a UGI domain. The separately evolved component or domain can be then fused to the remaining portions of the base editor within a cell by separately express both the evolved portion and the remaining non-evolved portions with split-intein polypeptide domains. The first portion could more broadly include any first amino acid portion of a base editor that is desired to be evolved using a continuous evolution method described herein. The second portion would in this embodiment refer to the remaining amino acid portion of the base editor that is not evolved using the herein methods. The evolved first portion and the second portion of the base editor could each be expressed with split-intein polypeptide domains in a cell. The natural protein splicing mechanisms of the cell would reassemble the evolved first portion and the non-evolved second portion to form a single fusion protein evolved base editor. The evolved first portion may comprise either the N- or C-terminal part of the single fusion protein. In an analogous manner, use of a second orthogonal trans-splicing intein pair could allow the evolved first portion to comprise an internal part of the single fusion protein.

Thus, any of the evolved and non-evolved components of the base editors herein described may be expressed with split-intein tags in order to facilitate the formation of a complete base editor comprising the evolved and non-evolved component within a cell.

The mechanism of the protein splicing process has been studied in great detail (Chong, et al., J. Biol. Chem. 1996, 271, 22159-22168; Xu, M-Q & Perler, F. B. EMBO Journal, 1996, 15, 5146-5153) and conserved amino acids have been found at the intein and extein splicing points (Xu, et al., EMBO Journal, 1994, 13 5517-522). The constructs described herein contain an intein sequence fused to the 5′-terminus of the first gene (e.g., the evolved portion of the base editor). Suitable intein sequences can be selected from any of the proteins known to contain protein splicing elements. A database containing all known inteins can be found on the World Wide Web (Perler, F. B. Nucleic Acids Research, 1999, 27, 346-347). The intein sequence is fused at the 3′ end to the 5′ end of a second gene. For targeting of this gene to a certain organelle, a peptide signal can be fused to the coding sequence of the gene. After the second gene, the intein-gene sequence can be repeated as often as desired for expression of multiple proteins in the same cell. For multi-intein containing constructs, it may be useful to use intein elements from different sources. After the sequence of the last gene to be expressed, a transcription termination sequence must be inserted. In one embodiment, a modified intein splicing unit is designed so that it can both catalyze excision of the exteins from the inteins as well as prevent ligation of the exteins. Mutagenesis of the C-terminal extein junction in the Pyrococcus species GB-D DNA polymerase was found to produce an altered splicing element that induces cleavage of exteins and inteins but prevents subsequent ligation of the exteins (Xu, M-Q & Perler, F. B. EMBO Journal, 1996, 15, 5146-5153). Mutation of serine 538 to either an alanine or glycine induced cleavage but prevented ligation. Mutation of equivalent residues in other intein splicing units should also prevent extein ligation due to the conservation of amino acids at the C-terminal extein junction to the intein. A preferred intein not containing an endonuclease domain is the Mycobacterium xenopi GyrA protein (Telenti, et al. J. Bacteriol. 1997, 179, 6378-6382). Others have been found in nature or have been created artificially by removing the endonuclease domains from endonuclease containing inteins (Chong, et al. J. Biol. Chem. 1997, 272, 15587-15590). In a preferred embodiment, the intein is selected so that it consists of the minimal number of amino acids needed to perform the splicing function, such as the intein from the Mycobacterium xenopi GyrA protein (Telenti, A., et al., J. Bacteriol. 1997, 179, 6378-6382). In an alternative embodiment, an intein without endonuclease activity is selected, such as the intein from the Mycobacterium xenopi GyrA protein or the Saccharomyces cerevisiae VMA intein that has been modified to remove endonuclease domains (Chong, 1997). Further modification of the intein splicing unit may allow the reaction rate of the cleavage reaction to be altered allowing protein dosage to be controlled by simply modifying the gene sequence of the splicing unit.

Inteins can also exist as two fragments encoded by two separately transcribed and translated genes. These so-called split inteins self-associate and catalyze protein-splicing activity in trans. Split inteins have been identified in diverse cyanobacteria and archaea (Caspi et al, Mol Microbiol. 50: 1569-1577 (2003); Choi J. et al, J Mol Biol. 556: 1093-1106 (2006.); Dassa B. et al, Biochemistry. 46:322-330 (2007.); Liu X. and Yang J., J Biol Chem. 275:26315-26318 (2003); Wu H. et al.

Proc Natl Acad Sci USA. £5:9226-9231 (1998.); and Zettler J. et al, FEBS Letters. 553:909-914 (2009)), but have not been found in eukaryotes thus far. Recently, a bioinformatic analysis of environmental metagenomic data revealed 26 different loci with a novel genomic arrangement. At each locus, a conserved enzyme coding region is interrupted by a split intein, with a freestanding endonuclease gene inserted between the sections coding for intein subdomains. Among them, five loci were completely assembled: DNA helicases (gp41-1, gp41-8); Inosine-5′-monophosphate dehydrogenase (IMPDH-1); and Ribonucleotide reductase catalytic subunits (NrdA-2 and NrdJ-1). This fractured gene organization appears to be present mainly in phages (Dassa et al, Nucleic Acids Research. 57:2560-2573 (2009)).

The split intein Npu DnaE was characterized as having the highest rate reported for the protein trans-splicing reaction. In addition, the Npu DnaE protein splicing reaction is considered robust and high-yielding with respect to different extein sequences, temperatures from 6 to 37° C., and the presence of up to 6M Urea (Zettler J. et al, FEBS Letters. 553:909-914 (2009); Iwai I. et al, FEBS Letters 550: 1853-1858 (2006)). As expected, when the Cysl Ala mutation at the N-domain of these inteins was introduced, the initial N to S-acyl shift and therefore protein splicing was blocked. Unfortunately, the C-terminal cleavage reaction was also almost completely inhibited. The dependence of the asparagine cyclization at the C-terminal splice junction on the acyl shift at the N-terminal scissile peptide bond seems to be a unique property common to the naturally split DnaE intein alleles (Zettler J. et al. FEBS Letters. 555:909-914 (2009)).

The mechanism of protein splicing typically has four steps [29-30]: 1) an N—S or N—O acyl shift at the intein N-terminus, which breaks the upstream peptide bond and forms an ester bond between the N-extein and the side chain of the intein's first amino acid (Cys or Ser); 2) a transesterification relocating the N-extein to the intein C-terminus, forming a new ester bond linking the N-extein to the side chain of the C-extein's first amino acid (Cys, Ser, or Thr); 3) Asn cyclization breaking the peptide bond between the intein and the C-extein; and 4) a S—N or O—N acyl shift that replaces the ester bond with a peptide bond between the N-extein and C-extein.

Protein trans-splicing, catalyzed by split inteins, provides an entirely enzymatic method for protein ligation [31]. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories [31-35]. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the −12 conserved beta-strands found in the structure of mini-inteins [25-28]. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

### K. Other Programmable Nucleases

In various embodiments described herein, the prime editors comprise a napDNAbp, such as a Cas9 protein. These proteins are “programmable” by way of their becoming complexed with a guide RNA (or a pegRNA, as the case may be), which guides the Cas9 protein to a target site on the DNA which possess a sequence that is complementary to the spacer portion of the gRNA (or pegRNA) and also which possesses the required PAM sequence. However, in certain embodiment envisioned here, the napDNAbp may be substituted with a different type of programmable protein, such as a zinc finger nuclease or a transcription activator-like effector nuclease (TALEN).

FIG. 1J depicts such a variation of prime editing contemplated herein that replaces the napDNAbp (e.g., SpCas9 nickase) with any programmable nuclease domain, such as zinc finger nucleases (ZFN) or transcription activator-like effector nucleases (TALEN). As such, it is contemplated that suitable nucleases do not necessarily need to be “programmed” by a nucleic acid targeting molecule (such as a guide RNA), but rather, may be programmed by defining the specificity of a DNA-binding domain, such as and in particular, a nuclease. Just as in prime editing with napDNAbp moieties, it is preferable that such alternative programmable nucleases be modified such that only one strand of a target DNA is cut. In other words, the programmable nucleases should function as nickases, preferably. Once a programmable nuclease is selected (e.g., a ZFN or a TALEN), then additional functionalities may be engineered into the system to allow it to operate in accordance with a prime editing-like mechanism. For example, the programmable nucleases may be modified by coupling (e.g., via a chemical linker) an RNA or DNA extension arm thereto, wherein the extension arm comprises a primer binding site (PBS) and a DNA synthesis template. The programmable nuclease may also be coupled (e.g., via a chemical or amino acid linker) to a polymerase, the nature of which will depend upon whether the extension arm is DNA or RNA. In the case of an RNA extension arm, the polymerase can be an RNA-dependent DNA polymerase (e.g., reverse transcriptase). In the case of a DNA extension arm, the polymerase can be a DNA-dependent DNA polymerase (e.g., a prokaryotic polymerase, including Pol I, Pol II, or Pol III, or a eukaryotic polymerase, including Pol a, Pol b, Pol g, Pol d, Pol e, or Pol z). The system may also include other functionalities added as fusions to the programmable nucleases, or added in trans to facilitate the reaction as a whole (e.g., (a) a helicase to unwind the DNA at the cut site to make the cut strand with the 3′ end available as a primer, (b) a FEN1 to help remove the endogenous strand on the cut strand to drive the reaction towards replacement of the endogenous strand with the synthesized strand, or (c) a nCas9:gRNA complex to create a second site nick on the opposite strand, which may help drive the integration of the synthesize repair through favored cellular repair of the non-edited strand). In an analogous manner to prime editing with a napDNAbp, such a complex with an otherwise programmable nuclease could be used to synthesize and then install a newly synthesized replacement strand of DNA carrying an edit of interest permanently into a target site of DNA.

Suitable alternative programmable nucleases are well known in the art which may be used in place of a napDNAbp:gRNA complex to construct an alternative prime editor system that can be programmed to selectively bind a target site of DNA, and which can be further modified in the manner described above to co-localize a polymerase and an RNA or DNA extension arm comprising a primer binding site and a DNA synthesis template to specific nick site. For example, and as represented in FIG. 1J, Transcription Activator-Like Effector Nucleases (TALENs) may be used as the programmable nuclease in the prime editing methods and compositions of matter described herein. TALENS are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. Nos. 12/965,590; 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety. In addition, TALENS are described in WO 2015/027134, U.S. Pat. No. 9,181,535, Boch et al., “Breaking the Code of DNA Binding Specificity of TAL-Type III Effectors”, Science, vol. 326, pp. 1509-1512 (2009), Bogdanove et al., TAL Effectors: Customizable Proteins for DNA Targeting, Science, vol. 333, pp. 1843-1846 (2011), Cade et al., “Highly efficient generation of heritable zebrafish gene mutations using homo- and heterodimeric TALENs”, Nucleic Acids Research, vol. 40, pp. 8001-8010 (2012), and Cermak et al., “Efficient design and assembly of custom TALEN and other TAL effector-based constructs for DNA targeting”, Nucleic Acids Research, vol. 39, No. 17, e82 (2011), each of which are incorporated herein by reference.

As represented in FIG. 1J, zinc finger nucleases may also be used as alternative programmable nucleases for use in prime editing in place of napDNAbps, such as Cas9 nickases. Like with TALENS, the ZFN proteins may be modified such that they function as nickases, i.e., engineering the ZFN such that it cleaves only one strand of the target DNA in a manner similar to the napDNAbp used with the prime editors described herein. ZFN proteins have been extensively described in the art, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

### [3] Polymerases (e.g., Reverse Transcriptases)

In various embodiments, the prime editor (PE) system disclosed herein includes a polymerase (e.g., DNA-dependent DNA polymerase or RNA-dependent DNA polymerase, such as, reverse transcriptase), or a variant thereof, which can be provided as a fusion protein with a napDNAbp or other programmable nuclease, or provide in trans.

Any polymerase may be used in the prime editors disclosed herein. The polymerases may be wild type polymerases, functional fragments, mutants, variants, or truncated variants, and the like. The polymerases may include wild type polymerases from eukaryotic, prokaryotic, archaeal, or viral organisms, and/or the polymerases may be modified by genetic engineering, mutagenesis, directed evolution-based processes. The polymerases may include T7 DNA polymerase, T5 DNA polymerase, T4 DNA polymerase, Klenow fragment DNA polymerase, DNA polymerase III and the like. The polymerases may also be thermostable, and may include Taq, Tne, Tma, Pfu, Tfl, Tth, Stoffel fragment, VENT® and DEEPVENT® DNA polymerases, KOD, Tgo, JDF3, and mutants, variants and derivatives thereof (see U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22(15):3259-3260 (1994), each of which are incorporated by reference). For synthesis of longer nucleic acid molecules (e.g., nucleic acid molecules longer than about 3-5 Kb in length), at least two DNA polymerases can be employed. In certain embodiments, one of the polymerases can be substantially lacking a 3′ exonuclease activity and the other may have a 3′ exonuclease activity. Such pairings may include polymerases that are the same or different. Examples of DNA polymerases substantially lacking in 3′ exonuclease activity include, but are not limited to, Taq, Tne(exo-), Tma(exo-), Pfu(exo-), Pwo(exo-), exo-KOD and Tth DNA polymerases, and mutants, variants and derivatives thereof.

Preferably, the polymerase usable in the prime editors disclosed herein are “template-dependent” polymerase (since the polymerases are intended to rely on the DNA synthesis template to specify the sequence of the DNA strand under synthesis during prime editing. As used herein, the term “template DNA molecule” refers to that strand of a nucleic acid from which a complementary nucleic acid strand is synthesized by a DNA polymerase, for example, in a primer extension reaction of the DNA synthesis template of a pegRNA.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)). The term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide. As such, in the case of prime editing, it can be said that the single strand of DNA synthesized by the polymerase of the prime editor against the DNA synthesis template is said to be “complementary” to the sequence of the DNA synthesis template.

### A. Exemplary Polymerases

In various embodiments, the prime editors described herein comprise a polymerase. The disclosure contemplates any wild type polymerase obtained from any naturally-occurring organism or virus, or obtained from a commercial or non-commercial source. In addition, the polymerases usable in the prime editors of the disclosure can include any naturally-occurring mutant polymerase, engineered mutant polymerase, or other variant polymerase, including truncated variants that retain function. The polymerases usable herein may also be engineered to contain specific amino acid substitutions, such as those specifically disclosed herein. In certain preferred embodiments, the polymerases usable in the prime editors of the disclosure are template-based polymerases, i.e., they synthesize nucleotide sequences in a template-dependent manner.

A polymerase is an enzyme that synthesizes a nucleotide strand and which may be used in connection with the prime editor systems described herein. The polymerases are preferably “template-dependent” polymerases (i.e., a polymerase which synthesizes a nucleotide strand based on the order of nucleotide bases of a template strand). In certain configurations, the polymerases can also be a “template-independent” (i.e., a polymerase which synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a pegRNA), and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

In some embodiments, the polymerases can be from bacteriophage. Bacteriophage DNA polymerases are generally devoid of 5′ to 3′ exonuclease activity, as this activity is encoded by a separate polypeptide. Examples of suitable DNA polymerases are T4, T7, and phi29 DNA polymerase. The enzymes available commercially are: T4 (available from many sources e.g., Epicentre) and T7 (available from many sources, e.g., Epicentre for unmodified and USB for 3′ to 5′ exo T7 “Sequenase” DNA polymerase).

The other embodiments, the polymerases are archaeal polymerases. There are 2 different classes of DNA polymerases which have been identified in archaea: 1. Family B/pol I type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol I or pol II) can be derived from archaea with optimal growth temperatures that are similar to the desired assay temperatures.

Thermostable archaeal DNA polymerases are isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to 3′ exonuclease activity, but in some cases 3′ to 5′ exonuclease activity is located in the same polypeptide.

There are a variety of commercially available Pol I DNA polymerases, some of which have been modified to reduce or abolish 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

Additional eubacteria related to those listed above are described in Thermophilic Bacteria (Kristjansson, J. K., ed.) CRC Press, Inc., Boca Raton, Fla., 1992.

The invention further provides for chimeric or non-chimeric DNA polymerases that are chemically modified according to methods disclosed in U.S. Pat. Nos. 5,677,152, 6,479,264 and 6,183,998, the contents of which are hereby incorporated by reference in their entirety.

Additional archaea DNA polymerases related to those listed above are described in the following references: Archaea: A Laboratory Manual (Robb, F. T. and Place, A. R., eds.), Cold Spring Harbor Laboratory Press, Cold Spring Harbor, N.Y., 1995 and Thermophilic Bacteria (Kristjansson, J. K., ed.) CRC Press, Inc., Boca Raton, Fla., 1992.

### B. Exemplary Reverse Transcriptases

In various embodiments, the prime editors described herein comprise a reverse transcriptase as the polymerase. The disclosure contemplates any wild type reverse transcriptase obtained from any naturally-occurring organism or virus, or obtained from a commercial or non-commercial source. In addition, the reverse transcriptases usable in the prime editors of the disclosure can include any naturally-occurring mutant RT, engineered mutant RT, or other variant RT, including truncated variants that retain function. The RTs may also be engineered to contain specific amino acid substitutions, such as those specifically disclosed herein.

Reverse transcriptases are multi-functional enzymes typically with three enzymatic activities including RNA- and DNA-dependent DNA polymerization activity, and an RNaseH activity that catalyzes the cleavage of RNA in RNA-DNA hybrids. Some mutants of reverse transcriptases have disabled the RNaseH moiety to prevent unintended damage to the mRNA. These enzymes that synthesize complementary DNA (cDNA) using mRNA as a template were first identified in RNA viruses. Subsequently, reverse transcriptases were isolated and purified directly from virus particles, cells or tissues. (e.g., see Kacian et al., 1971, Biochim. Biophys. Acta 46: 365-83; Yang et al., 1972, Biochem. Biophys. Res. Comm. 47: 505-11; Gerard et al., 1975, J. Virol. 15: 785-97; Liu et al., 1977, Arch. Virol. 55 187-200; Kato et al., 1984, J. Virol. Methods 9: 325-39; Luke et al., 1990, Biochem. 29: 1764-69 and Le Grice et al., 1991, J. Virol. 65: 7004-07, each of which are incorporated by reference). More recently, mutants and fusion proteins have been created in the quest for improved properties such as thermostability, fidelity and activity. Any of the wild type, variant, and/or mutant forms of reverse transcriptase which are known in the art or which can be made using methods known in the art are contemplated herein.

The reverse transcriptase (RT) gene (or the genetic information contained therein) can be obtained from a number of different sources. For instance, the gene may be obtained from eukaryotic cells which are infected with retrovirus, or from a number of plasmids which contain either a portion of or the entire retrovirus genome. In addition, messenger RNA-like RNA which contains the RT gene can be obtained from retroviruses. Examples of sources for RT include, but are not limited to, Moloney murine leukemia virus (M-MLV or MLVRT); human T-cell leukemia virus type 1 (HTLV-1); bovine leukemia virus (BLV); Rous Sarcoma Virus (RSV); human immunodeficiency virus (HIV); yeast, including Saccharomyces, Neurospora, Drosophila; primates; and rodents. See, for example, Weiss, et al., U.S. Pat. No. 4,663,290 (1987); Gerard, G. R., DNA:271-79 (1986); Kotewicz, M. L., et al., Gene 35:249-58 (1985); Tanese, N., et al., Proc. Natl. Acad. Sci. (USA):4944-48 (1985); Roth, M. J., at al., J. Biol. Chem. 260:9326-35 (1985); Michel, F., et al., Nature 316:641-43 (1985); Akins, R. A., et al., Cell 47:505-16 (1986), EMBO J. 4:1267-75 (1985); and Fawcett, D. F., Cell 47:1007-15 (1986) (each of which are incorporated herein by reference in their entireties).

### Wild Type RTs

Exemplary enzymes for use with the herein disclosed prime editors can include, but are not limited to, M-MLV reverse transcriptase and RSV reverse transcriptase. Enzymes having reverse transcriptase activity are commercially available. In certain embodiments, the reverse transcriptase provided in trans to the other components of the prime editor (PE) system. That is, the reverse transcriptase is expressed or otherwise provided as an individual component, i.e., not as a fusion protein with a napDNAbp.

A person of ordinary skill in the art will recognize that wild type reverse transcriptases, including but not limited to, Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

Exemplary wild type RT enzymes are as follows:

| DESCRIPTION | SEQUENCE | SEQ ID NO: |
|---|---|---|
| REVERSE | TLNIEDEYRLHETSKEPDVSLGSTWLSDFPQAWAETGG | SEQ ID NO: |
| TRANSCRIPTASE | MGLAVRQAPLIIPLKATSTPVSIKQYPMSQEARLGIKPHI | 32 |
| (M-MLV RT) | QRLLDQGILVPCQSPWNTPLLPVKKPGTNDYRPVQDLR | |
| WILD TYPE | EVNKRVEDIHPTVPNPYNLLSGLPPSHQWYTVLDLKDA | |
| MOLONEY | FFCLRLHPTSQPLFAFEWRDPEMGISGQLTWTRLPQGFK | |
| MURINE | NSPTLFDEALHRDLADFRIQHPDLILLQYVDDLLLAATSE | |
| LEUKEMIA | LDCQQGTRALLQTLGNLGYRASAKKAQICQKQVKYLG | |
| VIRUS | YLLKEGQRWLTEARKETVMGQPTPKTPRQLREFLGTAG | |
| USED IN PE1 | FCRLWIPGFAEMAAPLYPLTKTGTLFNWGPDQQKAYQEI | |
| (PRIME | KQALLTAPALGLPDLTKPFELFVDEKQGYAKGVLTQKLG | |
| EDITOR 1 | PWRRPVAYLSKKLDPVAAGWPPCLRMVAAIAVLTKDAG | |
| FUSION | KLTMGQPLVILAPHAVEALVKQPPDRWLSNARMTHYQA | |
| PROTEIN | LLLDTDRVQFGPVVALNPATLLPLPEEGLQHNCLDILAEA | |
| DISCLOSED | HGTRPDLTDQPLPDADHTWYTDGSSLLQEGQRKAGAAV | |
| HEREIN) | TTETEVIWAKALPAGTSAQRAELIALTQALKMAEGKKL | |
| | NVYTDSRYAFATAHIHGEIYRRRGLLTSEGKEIKNKDEIL | |
| | ALLKALFLPKRLSIIHCPGHQKGHSAEARGNRMADQAA | |
| | RKAAITETPDTSTLLIENSSP | |
| REVERSE | AFPLERPDWDYTTQAGRNHLVHYRQLLLAGLQNAGRS | SEQ ID NO: |
| TRANSCRIPTASE | PTNLAKVKGITQGPNESPSAFLERLKEAYRRYTPYDPED | 102 |
| MOLONEY | PGQETNVSMSFIWQSAPDIGRKLGRLEDLKSKTLGDLVR | |
| MURINE | EAEKIFNKRETPEEREERIRRETEEKEERRRTVDEQKEKE | |
| LEUKEMIA | RDRRRHREMSKLLATVVIGQEQDRQEGERKRPQLDKDQ | |
| VIRUS | CAYCKEKGHWAKDCPKKPRGPRGPRPQTSLLTLGDXGG | |
| REF SEQ. | QGQDPPPEPRITLKVGGQPVTFLVDTGAQHSVLTQNPGP | |
| AAA66622.1 | LSDKSAWVQGATGGKRYRWTTDRKVHLATGKVTHSFL | |
| | HVPDCPYPLLGRDLLTKLKAQIHFEGSGAQVVGPMGQP | |
| | LQVLTLNIEDEYRLHETSKEPDVSLGFTWLSDFPQAWAE | |
| | SGGMGLAVRQAPLIIPLKATSTPVSIKQYPMSQEARLGIK | |
| | PHIQRLLDQGILVPCQSPWNTPLLPVKKPGTNDYRPVQD | |
| | LREVNKRVEDIHPTVPNPYNLLSGLPPSHQWYTVLDLK | |
| | DAFFCLRLHPTSQPLFAFEWRDPEMGISGQLTWTRLPQG | |
| | FKNSPTLFDEALHRDLADFR | |
| REVERSE | TLQLEEEYRLFEPESTQKQEMDIWLKNFPQAWAETGGM | SEQ ID NO: |
| TRANSCRIPTASE | GTAHCQAPVLIQLKATATPISIRQYPMPHEAYQGIKPHIRR | 103 |
| FELINE | MLDQGILKPCQSPWNTPLLPVKKPGTEDYRPVQDLREV | |
| LEUKEMIA | NKRVEDIHPTVPNPYNLLSTLPPSHPWYTVLDLKDAFFC | |
| VIRUS | LRLHSESQLLFAFEWRDPEIGLSGQLTWTRLPQGFKNSPT | |
| REF SEQ. | LFDEALHSDLADFRVRYPALVLLQYVDDLLLAAATRTEC | |
| NP955579.1 | LEGTKALLETLGNKGYRASAKKAQICLQEVTYLGYSLK | |
| | DGQRWLTKARKEAILSIPVPKNSRQVREFLGTAGYCRLW | |
| | IPGFAELAAPLYPLTRPGTLFQWGTEQQLAFEDIKKALLS | |
| | SPALGLPDITKPFELFIDENSGFAKGVLVQKLGPWKRPVA | |
| | YLSKKLDTVASGWPPCLRMVAAIAILVKDAGKLTLGQPL | |
| | TILTSHPVEALVRQPPNKWLSNARMTHYQAMLLDAERV | |
| | HFGPTVSLNPATLLPLPSGGNHHDCLQILAETHGTRPDLT | |
| | DQPLPDADLTWYTDGSSFIRNGEREAGAAVTTESEVIWA | |
| | APLPPGTSAQRAELIALTQALKMAEGKKLTVYTDSRYAF | |
| | ATTHVHGEIYRRRGLLTSEGKEIKNKNEILALLEALFLPK | |
| | RLSIIHCPGHQKGDSPQAKGNRLADDTAKKAATETHSSL | |
| | TVL | |
| REVERSE | PISPIETVPVKLKPGMDGPKVKQWPLTEEKIKALVEICTE | SEQ ID NO: |
| TRANSCRIPTASE | MEKEGKISKIGPENPYNTPVFAIKKKDSTKWRKLVDFRE | 104 |
| HIV-1 RT, | LNKRTQDFWEVQLGIPHPAGLKKKKSVTVLDVGDAYFS | |
| CHAIN A | VPLDEDFRKYTAFTIPSINNETPGIRYQYNVLPQGWKGSP | |
| REF SEQ. ITL3- | AIFQSSMTKILEPFRKQNPDIVIYQYMDDLYVGSDLEIGQ | |
| A | HRTKIEELRQHLLRWGLTTPDKKHQKEPPFLWMGYELH | |
| | PDKWTVQPIVLPEKDSWTVNDIQKLVGKLNWASQIYPGI | |
| | KVRQLXKLLRGTKALTEVIPLTEEAELELAENREILKEPV | |
| | HGVYYDPSKDLIAEIQKQGQGQWTYQIYQEPFKNLKTG | |
| | KYARMRGAHTNDVKQLTEAVQKITTESIVIWGKTPKFKL | |
| | PIQKETWETWWTEYWQATWIPEWEFVNTPPLVKLWYQ | |
| | LEKEPIVGAETFYVDGAANRETKLGKAGYVTNRGRQK | |
| | VVTLTDTTNQKTELQAIYLALQDSGLEVNIVTDSQYALG | |
| | IIQAQPDQSESELVNQIIEQLIKKEKVYLAWVPAHKGIGG | |
| | NEQVDKLVSAGIRKV | |
| | SEE MARTINELLI ET AL., VIROLOGY, 1990, 174(1): 135- | |
| | 144, WHICH IS INCORPORATED BY REFERENCE | |
| REVERSE | PISPIETVPVKLKPGMDGPKVKQWPLTEEKIKALVEICTE | SEQ ID NO: |
| TRANSCRIPTASE | MEKEGKISKIGPENPYNTPVFAIKKKDSTKWRKLVDFRE | 105 |
| HIV-1 RT, | LNKRTQDFWEVQLGIPHPAGLKKKKSVTVLDVGDAYFS | |
| CHAIN B | VPLDEDFRKYTAFTIPSINNETPGIRYQYNVLPQGWKGSP | |
| REF SEQ. ITL3- | AIFQSSMTKILEPFRKQNPDIVIYQYMDDLYVGSDLEIGQ | |
| B | HRTKIEELRQHLLRWGLTTPDKKHQKEPPFLWMGYELH | |
| | PDKWTVQPIVLPEKDSWTVNDIQKLVGKLNWASQIYPGI | |
| | KVRQLCKLLRGTKALTEVIPLTEEAELELAENREILKEPV | |
| | HGVYYDPSKDLIAEIQKQGQGQWTYQIYQEPFKNLKTG | |
| | KYARMRGAHTNDVKQLTEAVQKITTESIVIWGKTPKFKL | |
| | PIQKETWETWWTEYWQATWIPEWEFVNTPPLVKLWYQ | |
| | LEKEPIVGAETF | |
| | SEE STAMMERS ET AL., J. MOL. BIOL., 1994, 242(4): 586- | |
| | 588, WHICH IS INCORPORATED BY REFERENCE | |
| REVERSE | TVALHLAIPLKWKPNHTPVWIDQWPLPEGKLVALTQLVE | SEQ ID NO: |
| TRANSCRIPTASE | KELQLGHIEPSLSCWNTPVFVIRKASGSYRLLHDLRAVN | 106 |
| ROUS | AKLVPFGAVQQGAPVLSALPRGWPLMVLDLKDCFFSIPL | |
| SARCOMA | AEQDREAFAFTLPSVNNQAPARRFQWKVLPQGMTCSPTI | |
| VIRUS RT | CQLIVGQILEPLRLKHPSLRMLHYMDDLLLAASSHDGLE | |
| REF SEQ. | AAGEEVISTLERAGFTISPDKVQKEPGVQYLGYKLGSTY | |
| ACL14945 | AAPVGLVAEPRIATLWDVQKLVGSLQWLRPALGIPPRLR | |
| | GPFYEQLRGSDPNEAREWNLDMKMAWREIVQLSTTAA | |
| | LERWDPALPLEGAVARCEQGAIGVLGQGLSTHPRPCLWL | |
| | FSTQPTKAFTAWLEVLTLLITKLRASAVRTFGKEVDILLL | |
| | PACFRDELPLPEGILLALRGFAGKIRSSDTPSIFDIARPLHV | |
| | SLKVRVTDHPVPGPTVFTDASSSTHKGVVVWREGPRWE | |
| | IKEIADLGASVQQLEARAVAMALLLWPTTPTNVVTDSAF | |
| | VAKMLLKMGQEGVPSTAAAFILEDALSQRSAMAAVLHV | |
| | RSHSEVPGFFTEGNDVADSQATFQAYPLREAKDLHTALH | |
| | IGPRALSKACNISMQQAREVVQTCPHCNSAPALEAGVNP | |
| | RGLGPLQIWQTDFTLEPRMAPRSWLAVTVDTASSAIVVT | |
| | QHGRVTSVAAQHHWATVIAVLGRPKAIKTDNGSCFTSKS | |
| | TREWLARWGIAHTTGIPGNSQGQAMVERANRLLKDKIR | |
| | VLAEGDGFMKRIPTSKQGELLAKAMYALNHFERGENTK | |
| | TPIQKHWRPTVLTEGPPVKIRIETGEWEKGWNVLVWGR | |
| | GYAAVKNRDTDKVIWVPSRKVKPDIAQKDEVTKKDEAS | |
| | PLFA | |
| | SEE YASUKAWA ET AL., J. BIOCHEM. 2009, 145(3): 315- | |
| | 324, WHICH IS INCORPORATED BY REFERENCE | |
| REVERSE | MMDHLLQKTQIQNQTEQVMNITNPNSIYIKGRLYFKGY | SEQ ID NO: |
| TRANSCRIPTASE | KKIELHCFVDTGASLCIASKFVIPEEHWINAERPIMVKIA | 107 |
| CAULIFLOWER | DGSSITINKVCRDIDLIIAGEIFHIPTVYQQESGIDFIIGNNF | |
| MOSAIC | CQLYEPFIQFTDRVIFTKDRTYPVHIAKLTRAVRVGTEGFL | |
| VIRUS RT | ESMKKRSKTQQPEPVNISTNKIAILSEGRRLSEEKLFITQQ | |
| REF SEQ. | RMQKIEELLEKVCSENPLDPNKTKQWMKASIKLSDPSK | |
| AGT42196 | AIKVKPMKYSPMDREEFDKQIKELLDLKVIKPSKSPHMA | |
| | PAFLVNNEAEKRRGKKRMVVNYKAMNKATVGDAYNLP | |
| | NKDELLTLIRGKKIFSSFDCKSGFWQVLLDQDSRPLTAFT | |
| | CPQGHYEWNVVPFGLKQAPSIFQRHMDEAFRVFRKFCC | |
| | VYVDDILVFSNNEEDHLLHVAMILQKCNQHGIILSKKKA | |
| | QLFKKKINFLGLEIDEGTHKPQGHILEHINKFPDTLEDKK | |
| | QLQRFLGILTYASDYIPKLAQIRKPLQAKLKENVPWKWT | |
| | KEDTLYMQKVKKNLQGFPPLHHPLPEEKLIIETDASDDY | |
| | WGGMLKAIKINEGTNTELICRYASGSFKAAEKNYHSND | |
| | KETLAVINTIKKFSIYLTPVHFLIRTDNTHFKSFVNLNYKG | |
| | DSKLGRNIRWQAWLSHYSFDVEHIKGTDNHFADFLSREF | |
| | NRVNS | |
| | SEE FARZADFAR ET AL., VIRUS GENES, 2013, 47(2): | |
| | 347-356, WHICH IS INCORPORATED BY REFERENCE | |
| REVERSE | MKEKISKIDKNFYTDIFIKTSFQNEFEAGGVIPPIAKNQVS | SEQ ID NO: |
| TRANSCRIPTASE | TISNKNKTFYSLAHSSPHYSIQTRIEKFLLKNIPLSASSFAF | 108 |
| KLEBSIELLA | RKERSYLHYLEPHTQNVKYCHLDIVSFFHSIDVNIVRDT | |
| PNEUMONIA | FSVYFSDEFLVKEKQSLLDAFMASVTLTAELDGVEKTFIP | |
| REF SEQ. | MGFKSSPSISNIIFRKIDILIQKFCDKNKITYTRYADDLLFS | |
| RFF81513.1 | TKKENNILSSTFFINEISSILSINKFKLNKSKYLYKEGTISL | |
| | GGYVIENILKDNSSGNIRLSSSKLNPLYKALYEIKKGSSS | |
| | KHICIKVFNLKLKRFIYKKNKEKFEAKFYSSQLKNKLLG | |
| | YRSYLLSFVIFHKKYKCINPIFLEKCVFLISEIESIMNRKF | |
| REVERSE | MKITSNNVTAVINGKGWHSINWKKCHQHVKTIQTRIAK | SEQ ID NO: |
| TRANSCRIPTASE | AACNQQWRTVGRLQRLLVRSFSARALAVKRVTENSGRK | 109 |
| ESCERICHIA | TPGVDGQIWSTPESKWEAIFKLRRKGYKPLPLKRVFIPKS | |
| COLI RT | NGKKRPLGIPVMLDRAMQALHLLGLEPVSETNADHNSY | |
| REF SEQ. | GFRPARCTADAIQQVCNMYSSRNASKWVLEGDIKGCFE | |
| TGH57013 | HISHEWLLENIPMDKQILRNWLKAGIIEKSIFSKTLSGTP | |
| | QGGIISPVLANMALDGLERLLQNRFGRNRLI | |
| REVERSE | MSKIKINYEKYHIKPFPHFDQRIKVNKKVKENLQNPFYI | SEQ ID NO: |
| TRANSCRIPTASE | AAHSFYPFIHYKKISYKFKNGTLSSPKERDIFYSGHMDG | 110 |
| BACILLUS | YIYKHYGEILNHKYNNTCIGKGIDHVSLAYRNNKMGKS | |
| SUBTILIS RT | NIHFAAEVINFISEQQQAFIFVSDFSSYFDSLDHAILKEKLI | |
| REF SEQ. | EVLEEQDKLSKDWWNVFKHITRYNWVEKEEVISDLECT | |
| QBJ66766 | KEKIARDKKSRERYYTPAEFREFRKRVNIKSNDTGVGIPQ | |
| | GTAISAVLANVYAIDLDQKLNQYALKYGGIYRRYSDDII | |
| | MVLPMTSDGQDPSNDHVSFIKSVVKRNKVTMGDSKTS | |
| | VLYYANNNIYEDYQRKRESKMDYLGFSFDGMTVKIREK | |
| | SLFKYYHRTYKKINSINWASVKKEKKVGRKKLYLLYSH | |
| | LGRNYKGHGNFISYCKKAHAVFEGNKKIESLINQQIKRH | |
| | WKKIQKRLVDV | |
| EUBACTERIUM | DTSNLMEQILSSDNLNRAYLQVVRNKGAEGVDGMKYT | SEQ ID NO: |
| RECTALE | ELKEHLAKNGETIKGQLRTRKYKPQPARRVEIPKPDGGV | 111 |
| GROUP II | RNLGVPTVTDRFIQQAIAQVLTPIYEEQFHDHSYGFRPNR | |
| INTRON RT | CAQQAILTALNIMNDGNDWIVDIDLEKFFDTVNHDKLM | |
| | TLIGRTIKDGDVISIVRKYLVSGIMIDDEYEDSIVGTPQGG | |
| | NLSPLLANIMLNELDKEMEKRGLNFVRYADDCIIMVGSE | |
| | MSANRVMRNISRFIEEKLGLKVNMTKSKVDRPSGLKYL | |
| | GFGFYFDPRAHQFKAKPHAKSVAKFKKRMKELTCRSW | |
| | GVSNSYKVEKLNQLIRGWINYFKIGSMKTLCKELDSRIR | |
| | YRLRMCIWKQWKTPQNQEKNLVKLGIDRNTARRVAYT | |
| | GKRIAYVCNKGAVNVAISNKRLASFGLISMLDYYIEKCV | |
| | TC | |
| GEOBACILLUS | ALLERILARDNLITALKRVEANQGAPGIDGVSTDQLRDYI | SEQ ID NO: |
| STEAROTHERMO- | RAHWSTIHAQLLAGTYRPAPVRRVEIPKPGGGTRQLGIP | 112 |
| PHILUS | TVVDRLIQQAILQELTPIFDPDFSSSSFGFRPGRNAHDAVR | |
| GROUP II | QAQGYIQEGYRYVVDMDLEKFFDRVNHDILMSRVARKV | |
| INTRON RT | KDKRVLKLIRAYLQAGVMIEGVKVQTEEGTPQGGPLSPL | |
| | LANILLDDLDKELEKRGLKFCRYADDCNIYVKSLRAGQ | |
| | RVKQSIQRFLEKTLKLKVNEEKSAVDRPWKRAFLGFSFT | |
| | PERKARIRLAPRSIQRLKQRIRQLTNPNWSISMPERIHRV | |
| | NQYVMGWIGYFRLVETPSVLQTIEGWIRRRLRLCQWLQ | |
| | WKRVRTRIRELRALGLKETAVMEIANTRKGAWRTTKTP | |
| | QLHQALGKTYWTAQGLKSLTQR | |

### Variant and Error-Prone RTs

Reverse transcriptases are essential for synthesizing complementary DNA (cDNA) strands from RNA templates. Reverse transcriptases are enzymes composed of distinct domains that exhibit different biochemical activities. The enzymes catalyze the synthesis of DNA from an RNA template, as follows: In the presence of an annealed primer, reverse transcriptase binds to an RNA template and initiates the polymerization reaction. RNA-dependent DNA polymerase activity synthesizes the complementary DNA (cDNA) strand, incorporating dNTPs. RNase H activity degrades the RNA template of the DNA:RNA complex. Thus, reverse transcriptases comprise (a) a binding activity that recognizes and binds to a RNA/DNA hybrid, (b) an RNA-dependent DNA polymerase activity, and (c) an RNase H activity. In addition, reverse transcriptases generally are regarded as having various attributes, including their thermostability, processivity (rate of dNTP incorporation), and fidelity (or error-rate). The reverse transcriptase variants contemplated herein may include any mutations to reverse transcriptase that impacts or changes any one or more of these enzymatic activities (e.g., RNA-dependent DNA polymerase activity, RNase H activity, or DNA/RNA hybrid-binding activity) or enzyme properties (e.g., thermostability, processivity, or fidelity). Such variants may be available in the art in the public domain, available commercially, or may be made using known methods of mutagenesis, including directed evolutionary processes (e.g., PACE or PANCE).

In various embodiments, the reverse transcriptase may be a variant reverse transcriptase. As used herein, a “variant reverse transcriptase” includes any naturally occurring or genetically engineered variant comprising one or more mutations (including singular mutations, inversions, deletions, insertions, and rearrangements) relative to a reference sequences (e.g., a reference wild type sequence). RT naturally have several activities, including an RNA-dependent DNA polymerase activity, ribonuclease H activity, and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. Variant RT's may comprise a mutation which impacts one or more of these activities (either which reduces or increases these activities, or which eliminates these activities all together). In addition, variant RTs may comprise one or more mutations which render the RT more or less stable, less prone to aggregation, and facilitates purification and/or detection, and/or other the modification of properties or characteristics.

A person of ordinary skill in the art will recognize that variant reverse transcriptases derived from other reverse transcriptases, including but not limited to Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

One method of preparing variant RTs is by genetic modification (e.g., by modifying the DNA sequence of a wild-type reverse transcriptase). A number of methods are known in the art that permit the random as well as targeted mutation of DNA sequences (see for example, Ausubel et. al. Short Protocols in Molecular Biology (1995) 3.sup.rd Ed. John Wiley & Sons, Inc.). In addition, there are a number of commercially available kits for site-directed mutagenesis, including both conventional and PCR-based methods. Examples include the QuikChange Site-Directed Mutagenesis Kits (AGILENT®), the Q5© Site-Directed Mutagenesis Kit (NEW ENGLAND BIOLABS®), and GeneArt™ Site-Directed Mutagenesis System (THERMOFISHER SCIENTIFIC®).

In addition, mutant reverse transcriptases may be generated by insertional mutation or truncation (N-terminal, internal, or C-terminal insertions or truncations) according to methodologies known to one skilled in the art. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Methods of random mutagenesis, which will result in a panel of mutants bearing one or more randomly situated mutations, exist in the art. Such a panel of mutants may then be screened for those exhibiting the desired properties, for example, increased stability, relative to a wild-type reverse transcriptase.

An example of a method for random mutagenesis is the so-called “error-prone PCR method.” As the name implies, the method amplifies a given sequence under conditions in which the DNA polymerase does not support high fidelity incorporation. Although the conditions encouraging error-prone incorporation for different DNA polymerases vary, one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

In various aspects, the RT of the prime editors may be an “error-prone” reverse transcriptase variant. Error-prone reverse transcriptases that are known and/or available in the art may be used. It will be appreciated that reverse transcriptases naturally do not have any proofreading function; thus the error rate of reverse transcriptase is generally higher than DNA polymerases comprising a proofreading activity. The error-rate of any particular reverse transcriptase is a property of the enzyme's “fidelity,” which represents the accuracy of template-directed polymerization of DNA against its RNA template. An RT with high fidelity has a low-error rate. Conversely, an RT with low fidelity has a high-error rate. The fidelity of M-MLV-based reverse transcriptases are reported to have an error rate in the range of one error in 15,000 to 27,000 nucleotides synthesized. See Boutabout et al., “DNA synthesis fidelity by the reverse transcriptase of the yeast retrotransposon Ty1,” Nucleic Acids Res, 2001, 29: 2217-2222, which is incorporated by reference. Thus, for purposes of this application, those reverse transcriptases considered to be “error-prone” or which are considered to have an “error-prone fidelity” are those having an error rate that is less than one error in 15,000 nucleotides synthesized.

Error-prone reverse transcriptase also may be created through mutagenesis of a starting RT enzyme (e.g., a wild type M-MLV RT). The method of mutagenesis is not limited and may include directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

Error-prone reverse transcriptases may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Other error-prone reverse transcriptases have been described in the literature, each of which are contemplated for use in the herein methods and compositions. For example, error-prone reverse transcriptases have been described in Bebenek et al., “Error-prone Polymerization by HIV-1 Reverse Transcriptase,” J Biol Chem, 1993, Vol. 268: 10324-10334 and Sebastian-Martin et al., “Transcriptional inaccuracy threshold attenuates differences in RNA-dependent DNA synthesis fidelity between retroviral reverse transcriptases,” Scientific Reports, 2018, Vol. 8: 627, each of which are incorporated by reference. Still further, reverse transcriptases, including error-prone reverse transcriptases can be obtained from a commercial supplier, including ProtoScript® (II) Reverse Transcriptase, AMV Reverse Transcriptase, WarmStart® Reverse Transcriptase, and M-MuLV Reverse Transcriptase, all from NEW ENGLAND BIOLABS®, or AMV Reverse Transcriptase XL, SMARTScribe Reverse Transcriptase, GPR ultra-pure MMLV Reverse Transcriptase, all from TAKARA BIO USA, INC. (formerly CLONTECH).

The herein disclosure also contemplates reverse transcriptases having mutations in RNaseH domain. As mentioned above, one of the intrinsic properties of reverse transcriptases is the RNase H activity, which cleaves the RNA template of the RNA:cDNA hybrid concurrently with polymerization. The RNase H activity can be undesirable for synthesis of long cDNAs because the RNA template may be degraded before completion of full-length reverse transcription. The RNase H activity may also lower reverse transcription efficiency, presumably due to its competition with the polymerase activity of the enzyme. Thus, the present disclosure contemplates any reverse transcriptase variants that comprise a modified RNaseH activity.

The herein disclosure also contemplates reverse transcriptases having mutations in the RNA-dependent DNA polymerase domain. As mentioned above, one of the intrinsic properties of reverse transcriptases is the RNA-dependent DNA polymerase activity, which incorporates the nucleobases into the nascent cDNA strand as coded by the template RNA strand of the RNA:cDNA hybrid. The RNA-dependent DNA polymerase activity can be increased or decreased (i.e., in terms of its rate of incorporation) to either increase or decrease the processivity of the enzyme. Thus, the present disclosure contemplates any reverse transcriptase variants that comprise a modified RNA-dependent DNA polymerase activity such that the processivity of the enzyme of either increased or decreased relative to an unmodified version.

Also contemplated herein are reverse transcriptase variants that have altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields, which can lead to an improved generation of the 3′ flap ssDNA as a result of the prime editing process. Wild type M-MLV reverse transcriptase typically has an optimal temperature in the range of 37-48° C.; however, mutations may be introduced that allow for the reverse transcription activity at higher temperatures of over 48° C., including 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., and higher.

The variant reverse transcriptases contemplated herein, including error-prone RTs, thermostable RTs, increase-processivity RTs, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

Variant RT enzymes used herein may also include other “RT variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference RT protein, including any wild type RT, or mutant RT, or fragment RT, or other variant of RT disclosed or contemplated herein or known in the art.

In some embodiments, an RT variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT. In some embodiments, the RT variant comprises a fragment of a reference RT, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference RT. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type RT (M-MLV reverse transcriptase) (e.g., SEQ ID NO: 32) or to any of the reverse transcriptases of SEQ ID NOs: 102-112.

In some embodiments, the disclosure also may utilize RT fragments which retain their functionality and which are fragments of any herein disclosed RT proteins. In some embodiments, the RT fragment is at least 100 amino acids in length. In some embodiments, the fragment is at least 100, 150, 200, 250, 300, 350, 400, 450, 500, 550, or up to 600 or more amino acids in length.

In still other embodiments, the disclosure also may utilize RT variants which are truncated at the N-terminus or the C-terminus, or both, by a certain number of amino acids which results in a truncated variant which still retains sufficient polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end of the protein. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end of the protein. In still other embodiments, the RT truncated variant has a truncation at the N-terminal and the C-terminal end which are the same or different lengths.

For example, the prime editors disclosed herein may include a truncated version of M-MLV reverse transcriptase. In this embodiment, the reverse transcriptase contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e., adeno-associated virus and lentivirus delivery). This embodiment is referred to as MMLV-RT(trunc) and has the following amino acid sequence:

| | |
|---|---|
| MMLV- | TLNIEDEYRLHETSKEPDVSLGSTWLSDFPQAWAETGGMGLA |
| RT | VRQAPLIIPLKATSTPVSIKQYPMSQEARLGIKPHIQRLLDQ |
| (TRUNC) | GILVPCQSPWNTPLLPVKKPGTNDYRPVQDLREVNKRVEDIH |
| | PTVPNPYNLLSGLPPSHQWYTVLDLKDAFFCLRLHPTSQPLF |
| | AFEWRDPEMGISGQLTWTRLPQGFKNSPTLFNEALHRDLADF |
| | RIQHPDLILLQYVDDLLLAATSELDCQQGTRALLQTLGNLGY |
| | RASAKKAQICQKQVKYLGYLLKEGQRWLTEARKETVMGQPTP |
| | KTPRQLREFLGKAGFCRLFIPGFAEMAAPLYPLTKPGTLFNW |
| | GPDQQKAYQEIKQALLTAPALGLPDLTKPFELFVDEKQGYAK |
| | GVLTQKLGPWRRPVAYLSKKLDPVAAGWPPCLRMVAAIAVLT |
| | KDAGKLTMGQPLVILAPHAVEALVKQPPDRWLSNARMTHYQA |
| | LLLDTDRVQFGPVVALNPATLLPLPEEGLQHNCLDNSRLIN |
| | (SEQ ID NO: 36) |

In various embodiments, the prime editors disclosed herein may comprise one of the RT variants described herein, or a RT variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

In still other embodiments, the present methods and compositions may utilize a DNA polymerase that has been evolved into a reverse transcriptase, as described in Effefson et al., “Synthetic evolutionary origin of a proofreading reverse transcriptase,” Science, Jun. 24, 2016, Vol. 352: 1590-1593, the contents of which are incorporated herein by reference.

In certain other embodiments, the reverse transcriptase is provided as a component of a fusion protein also comprising a napDNAbp. In other words, in some embodiments, the reverse transcriptase is fused to a napDNAbp as a fusion protein.

In various embodiments, variant reverse transcriptases can be engineered from wild type M-MLV reverse transcriptase as represented by SEQ ID NO: 32.

In various embodiments, the prime editors described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 32 or at a corresponding amino acid position in another wild type RT polypeptide sequence.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes:

| Description | Sequence (variant substitutions relative to wild type) | SEQ ID NO: |
|---|---|---|
| Reverse | TLNIEDEYRLHETSKEPDVSLGSTWLSDFPQAWAETGG | SEQ ID NO: |
| transcriptase | MGLAVRQAPLIIPLKATSTPVSIKQYPMSQEARLGIKPHI | 32 |
| (M-MLV | QRLLDQGILVPCQSPWNTPLLPVKKPGTNDYRPVQDLR | |
| RT) wild | EVNKRVEDIHPTVPNPYNLLSGLPPSHQWYTVLDLKDA | |
| type | FFCLRLHPTSQPLFAFEWRDPEMGISGQLTWTRLPQGFK | |
| moloney | NSPTLFDEALHRDLADFRIQHPDLILLQYVDDLLLAATS | |
| murine | ELDCQQGTRALLQTLGNLGYRASAKKAQICQKQVKYL | |
| leukemia | GYLLKEGQRWLTEARKETVMGQPTPKTPRQLREFLGT | |
| virus | AGFCRLWIPGFAEMAAPLYPLTKTGTLFNWGPDQQKA | |
| Used in | YQEIKQALLTAPALGLPDLTKPFELFVDEKQGYAKGVL | |
| PE1 (prime | TQKLGPWRRPVAYLSKKLDPVAAGWPPCLRMVAAIAV | |
| editor 1 | LTKDAGKLTMGQPLVILAPHAVEALVKQPPDRWLSNA | |
| fusion | RMTHYQALLLDTDRVQFGPVVALNPATLLPLPEEGLQH | |
| protein | NCLDILAEAHGTRPDLTDQPLPDADHTWYTDGSSLLQE | |
| disclosed | GQRKAGAAVTTETEVIWAKALPAGTSAQRAELIALTQA | |
| herein) | LKMAEGKKLNVYTDSRYAFATAHIHGEIYRRRGLLTSE | |
| | GKEIKNKDEILALLKALFLPKRLSIIHCPGHQKGHSAEA | |
| | RGNRMADQAARKAAITETPDTSTLLIENSSP (SEQ ID | |
| | NO: 32) | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 113 |
| | QRLLDQGILVPCQSPWNTPLLPVKKPGTNDYRPVQDLR | |
| | EVNKRVEDIHPTVPNPYNLLSGLPPSHQWYTVLDLKDA | |
| | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKTGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGLLTSE | |
| | GKEIKNKDEILALLKALFLPKRLSIIHCPGHQKGHSAEA | |
| | RGNRMADQAARKAAITETPDTSTLLIENSSP (SEQ ID | |
| | NO: 701) | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 114 |
| T330P | QRLLDQGILVPCQSPWNTPLLPVKKPGTNDYRPVQDLR | |
| | EVNKRVEDIHPTVPNPYNLLSGLPPSHQWYTVLDLKDA | |
| | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGLLTSE | |
| | GKEIKNKDEILALLKALFLPKRLSIIHCPGHQKGHSAEA | |
| | RGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 115 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQKARLGIKPHI | 116 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| E69K | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 117 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| E302R | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLRRFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 118 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| E607K | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | KGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 119 |
| T330P | QRLLDQGILVPCQSPWNTPLLPVKKPGTNDYRPVQDLR | |
| L603W | EVNKRVEDIHPTVPNPYNLLSGPPPSHQWYTVLDLKDA | |
| L139P | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 120 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| L435G | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVIGAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 121 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| N454K | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSKA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 122 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| T306K | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGK | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 123 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| W313F | FFCLRLHPTSQPLFAFEWRDPEMGISGQLTWTRLPQGFK | |
| | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLFIPGFAEMAAPLYPLTKPGTLFNWGPDQQKAY | |
| | QEIKQALLTAPALGLPDLTKPFELFVDEKQGYAKGVLT | |
| | QKLGPWRRPVAYLSKKLDPVAAGWPPCLRMVAAIAVL | |
| | TKDAGKLTMGQPLVILAPHAVEALVKQPPDRWLSNAR | |
| | MTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 124 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| D524G | FFCLRLHPTSQPLFAFEWRDPEMGISGQLTWTRLPQGFK | |
| E562Q | NSPTLFNEALHRDLADFRIQHPDLILLQYVDDLLLAATS | |
| D583N | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTGGSSLLQE | |
| | GQRKAGAAVTTETEVIWAKALPAGTSAQRAQLIALTQ | |
| | ALKMAEGKKLNVYTNSRYAFATAHIHGEIYRRRGWLT | |
| | SEGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 125 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| E302R | FFCLRLHPTSQPLFAFEWRDPEMGISGQLTWTRLPQGFK | |
| W313F | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLRRFLGT | |
| | AGFCRLFIPGFAEMAAPLYPLTKPGTLFNWGPDQQKAY | |
| | QEIKQALLTAPALGLPDLTKPFELFVDEKQGYAKGVLT | |
| | QKLGPWRRPVAYLSKKLDPVAAGWPPCLRMVAAIAVL | |
| | TKDAGKLTMGQPLVILAPHAVEALVKQPPDRWLSNAR | |
| | MTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 126 |
| T330P | QRLLDQGILVPCQSPWNTPLLPVKKPGTNDYRPVQDLR | |
| L603W | EVNKRVEDIHPTVPNPYNLLSGPPPSHQWYTVLDLKDA | |
| E607K | FFCLRLHPTSQPLFAFEWRDPEMGISGQLTWTRLPQGFK | |
| L139P | NSPTLFNEALHRDLADFRIQHPDLILLQYVDDLLLAATS | |
| | ELDCQQGTRALLQTLGNLGYRASAKKAQICQKQVKYL | |
| | GYLLKEGQRWLTEARKETVMGQPTPKTPRQLREFLGT | |
| | AGFCRLWIPGFAEMAAPLYPLTKPGTLFNWGPDQQKA | |
| | YQEIKQALLTAPALGLPDLTKPFELFVDEKQGYAKGVL | |
| | TQKLGPWRRPVAYLSKKLDPVAAGWPPCLRMVAAIAV | |
| | LTKDAGKLTMGQPLVILAPHAVEALVKQPPDRWLSNA | |
| | RMTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | KGKEIKNKDEILALLKALFLPKRLSIIHCPGHQKGHSAE | |
| | ARGNRMADQAARKAAITETPDTSTLLIENSSP | |
| M-MLV RT | TLNIEDEYRLHETSKEPDVSLGSTWLSDFPQAWAETGG | SEQ ID NO: |
| P51L S67K | MGLAVRQAPLIILLKATSTPVSIKQYPMKQEARLGIKPH | 127 |
| T197A | IQRLLDQGILVPCQSPWNTPLLPVKKPGTNDYRPVQDL | |
| H204R | REVNKRVEDIHPTVPNPYNLLSGLPPSHQWYTVLDLKD | |
| E302K | AFFCLRLHPTSQPLFAFEWRDPEMGISGQLTWTRLPQGF | |
| F309N | KNSPALFDEALRRDLADFRIQHPDLILLQYVDDLLLAAT | |
| W313F | SELDCQQGTRALLQTLGNLGYRASAKKAQICQKQVKY | |
| T330P | LGYLLKEGQRWLTEARKETVMGQPTPKTPRQLRKFLG | |
| L435G | TAGNCRLFIPGFAEMAAPLYPLTKPGTLFNWGPDQQKA | |
| N454K | YQEIKQALLTAPALGLPDLTKPFELFVDEKQGYAKGVL | |
| D524G | TQKLGPWRRPVAYLSKKLDPVAAGWPPCLRMVAAIAV | |
| D583N | LTKDAGKLTMGQPLVIGAPHAVEALVKQPPDRWLSKA | |
| H594Q | RMTHYQALLLDTDRVQFGPVVALNPATLLPLPEEGLQH | |
| D653N | NCLDILAEAHGTRPDLTDQPLPDADHTWYTGGSSLLQE | |
| | GQRKAGAAVTTETEVIWAKALPAGTSAQRAELIALTQA | |
| | LKMAEGKKLNVYTNSRYAFATAHIQGEIYRRRGLLTSE | |
| | GKEIKNKDEILALLKALFLPKRLSIIHCPGHQKGHSAEA | |
| | RGNRMANQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIILLKATSTPVSIKQYPMKQEARLGIKPH | 128 |
| P51L | IQRLLDQGILVPCQSPWNTPLLPVKKPGTNDYRPVQDL | |
| S67K | REVNKRVEDIHPTVPNPYNLLSGLPPSHQWYTVLDLKD | |
| T197A | AFFCLRLHPTSQPLFAFEWRDPEMGISGQLTWTRLPQGF | |
| H204R | KNSPALFNEALRRDLADFRIQHPDLILLQYVDDLLLAAT | |
| E302K | SELDCQQGTRALLQTLGNLGYRASAKKAQICQKQVKY | |
| F309N | LGYLLKEGQRWLTEARKETVMGQPTPKTPRQLRKFLG | |
| W313F | TAGNCRLFIPGFAEMAAPLYPLTKPGTLFNWGPDQQKA | |
| T330P | YQEIKQALLTAPALGLPDLTKPFELFVDEKQGYAKGVL | |
| L345G | TQKLGPWRRPVAYLSKKLDPVAAGWPPCLRMVAAIAV | |
| N454K | LTKDAGKLTMGQPLVIGAPHAVEALVKQPPDRWLSKA | |
| D524G | RMTHYQALLLDTDRVQFGPVVALNPATLLPLPEEGLQH | |
| D583N | NCLDILAEAHGTRPDLTDQPLPDADHTWYTGGSSLLQE | |
| H594Q | GQRKAGAAVTTETEVIWAKALPAGTSAQRAELIALTQA | |
| D653N | LKMAEGKKLNVYTNSRYAFATAHIQGEIYRRRGLLTSE | |
| | GKEIKNKDEILALLKALFLPKRLSIIHCPGHQKGHSAEA | |
| | RGNRMANQAARKAAITETPDTSTLLIENSSP | |
| M-MLV RT | TLNIEDEYRLHETSKEPDVSLGSTWLSDFPQAWAETGG | SEQ ID NO: |
| D200N | MGLAVRQAPLIIPLKATSTPVSIKQYPMSQEARLGIKPHI | 34 |
| T330P | QRLLDQGILVPCQSPWNTPLLPVKKPGTNDYRPVQDLR | |
| L603W | EVNKRVEDIHPTVPNPYNLLSGLPPSHQWYTVLDLKDA | |
| T306K | FFCLRLHPTSQPLFAFEWRDPEMGISGQLTWTRLPQGFK | |
| W313F | NSPTLFNEALHRDLADFRIQHPDLILLQYVDDLLLAATS | |
| in PE2 | ELDCQQGTRALLQTLGNLGYRASAKKAQICQKQVKYL | |
| | GYLLKEGQRWLTEARKETVMGQPTPKTPRQLREFLGK | |
| | AGFCRLFIPGFAEMAAPLYPLTKPGTLFNWGPDQQKAY | |
| | QEIKQALLTAPALGLPDLTKPFELFVDEKQGYAKGVLT | |
| | QKLGPWRRPVAYLSKKLDPVAAGWPPCLRMVAAIAVL | |
| | TKDAGKLTMGQPLVILAPHAVEALVKQPPDRWLSNAR | |
| | MTHYQALLLDTDRVQFGPVVALNPATLLPLPEEGLQH | |
| | NCLDILAEAHGTRPDLTDQPLPDADHTWYTDGSSLLQE | |
| | GQRKAGAAVTTETEVIWAKALPAGTSAQRAELIALTQA | |
| | LKMAEGKKLNVYTDSRYAFATAHIHGEIYRRRGWLTS | |
| | EGKEIKNKDEILALLKALFLPKRLSIIHCPGHQKGHSAE | |
| | ARGNRMADQAARKAAITETPDTSTLLIENSSP | |

In various other embodiments, the prime editors described herein with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 32 or at a corresponding amino acid position in another wild type RT polypeptide sequence, wherein “X” can be any amino acid.

In various other embodiments, the prime editors described herein (with RT provided as either a fusion partner or in trans) can include a variant RT comprising a P51X mutation in the wild type M-MLV RT of SEQ ID NO: 32 or at a corresponding amino acid position in another wild type RT polypeptide sequence, wherein “X” can be any amino acid. In certain embodiments, X is L.

In various other embodiments, the prime editors described herein (with RT provided as either a fusion partner or in trans) can include a variant RT comprising a S67X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a E69X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a L139X mutation in the wild type M-MLV RT of SEQ ID NO: 32 or at a corresponding amino acid position in another wild type RT polypeptide sequence, wherein “X” can be any amino acid. In certain embodiments, X is P.

In various other embodiments, the prime editors described herein (with RT provided as either a fusion partner or in trans) can include a variant RT comprising a T197X mutation in the wild type M-MLV RT of SEQ ID NO: 32 or at a corresponding amino acid position in another wild type RT polypeptide sequence, wherein “X” can be any amino acid. In certain embodiments, X is A.

In various other embodiments, the prime editors described herein (with RT provided as either a fusion partner or in trans) can include a variant RT comprising a D200X mutation in the wild type M-MLV RT of SEQ ID NO: 32 or at a corresponding amino acid position in another wild type RT polypeptide sequence, wherein “X” can be any amino acid. In certain embodiments, X is N.

In various other embodiments, the prime editors described herein (with RT provided as either a fusion partner or in trans) can include a variant RT comprising a H204X mutation in the wild type M-MLV RT of SEQ ID NO: 32 or at a corresponding amino acid position in another wild type RT polypeptide sequence, wherein “X” can be any amino acid. In certain embodiments, X is R.

In various other embodiments, the prime editors described herein (with RT provided as either a fusion partner or in trans) can include a variant RT comprising a F209X mutation in the wild type M-MLV RT of SEQ ID NO: 32 or at a corresponding amino acid position in another wild type RT polypeptide sequence, wherein “X” can be any amino acid. In certain embodiments, X is N.

In various other embodiments, the prime editors described herein (with RT provided as either a fusion partner or in trans) can include a variant RT comprising a E302X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a E302X mutation in the wild type M-MLV RT of SEQ ID NO: 32 or at a corresponding amino acid position in another wild type RT polypeptide sequence, wherein “X” can be any amino acid. In certain embodiments, X is R.

In various other embodiments, the prime editors described herein (with RT provided as either a fusion partner or in trans) can include a variant RT comprising a T306X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a F309X mutation in the wild type M-MLV RT of SEQ ID NO: 32 or at a corresponding amino acid position in another wild type RT polypeptide sequence, wherein “X” can be any amino acid. In certain embodiments, X is N.

In various other embodiments, the prime editors described herein (with RT provided as either a fusion partner or in trans) can include a variant RT comprising a W313X mutation in the wild type M-MLV RT of SEQ ID NO: 32 or at a corresponding amino acid position in another wild type RT polypeptide sequence, wherein “X” can be any amino acid. In certain embodiments, X is F.

In various other embodiments, the prime editors described herein (with RT provided as either a fusion partner or in trans) can include a variant RT comprising a T330X mutation in the wild type M-MLV RT of SEQ ID NO: 32 or at a corresponding amino acid position in another wild type RT polypeptide sequence, wherein “X” can be any amino acid. In certain embodiments, X is P.

In various other embodiments, the prime editors described herein (with RT provided as either a fusion partner or in trans) can include a variant RT comprising a L345X mutation in the wild type M-MLV RT of SEQ ID NO: 32 or at a corresponding amino acid position in another wild type RT polypeptide sequence, wherein “X” can be any amino acid. In certain embodiments, X is G.

In various other embodiments, the prime editors described herein (with RT provided as either a fusion partner or in trans) can include a variant RT comprising a L435X mutation in the wild type M-MLV RT of SEQ ID NO: 32 or at a corresponding amino acid position in another wild type RT polypeptide sequence, wherein “X” can be any amino acid. In certain embodiments, X is G.

In various other embodiments, the prime editors described herein (with RT provided as either a fusion partner or in trans) can include a variant RT comprising a N454X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a D524X mutation in the wild type M-MLV RT of SEQ ID NO: 32 or at a corresponding amino acid position in another wild type RT polypeptide sequence, wherein “X” can be any amino acid. In certain embodiments, X is G.

In various other embodiments, the prime editors described herein (with RT provided as either a fusion partner or in trans) can include a variant RT comprising a E562X mutation in the wild type M-MLV RT of SEQ ID NO: 32 or at a corresponding amino acid position in another wild type RT polypeptide sequence, wherein “X” can be any amino acid. In certain embodiments, X is Q.

In various other embodiments, the prime editors described herein (with RT provided as either a fusion partner or in trans) can include a variant RT comprising a D583X mutation in the wild type M-MLV RT of SEQ ID NO: 32 or at a corresponding amino acid position in another wild type RT polypeptide sequence, wherein “X” can be any amino acid. In certain embodiments, X is N.

In various other embodiments, the prime editors described herein (with RT provided as either a fusion partner or in trans) can include a variant RT comprising a H594X mutation in the wild type M-MLV RT of SEQ ID NO: 32 or at a corresponding amino acid position in another wild type RT polypeptide sequence, wherein “X” can be any amino acid. In certain embodiments, X is Q.

In various other embodiments, the prime editors described herein (with RT provided as either a fusion partner or in trans) can include a variant RT comprising a L603X mutation in the wild type M-MLV RT of SEQ ID NO: 32 or at a corresponding amino acid position in another wild type RT polypeptide sequence, wherein “X” can be any amino acid. In certain embodiments, X is W.

In various other embodiments, the prime editors described herein (with RT provided as either a fusion partner or in trans) can include a variant RT comprising a E607X mutation in the wild type M-MLV RT of SEQ ID NO: 32 or at a corresponding amino acid position in another wild type RT polypeptide sequence, wherein “X” can be any amino acid. In certain embodiments, X is K.

In various other embodiments, the prime editors described herein (with RT provided as either a fusion partner or in trans) can include a variant RT comprising a D653X mutation in the wild type M-MLV RT of SEQ ID NO: 32 or at a corresponding amino acid position in another wild type RT polypeptide sequence, wherein “X” can be any amino acid. In certain embodiments, X is N.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95% or at least 99% sequence identity to the wild-type enzymes or partial enzymes represented by SEQ ID NOs: 32, 34, 113-128. The prime editor (PE) system described here contemplates any publicly-available reverse transcriptase described or disclosed in any of the following U.S. patents (each of which are incorporated by reference in their entireties): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references describe reverse transcriptases in art. Each of their disclosures are incorporated herein by reference in their entireties.
Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).
Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).
Zhao, C., Liu, F. & Pyle, A. M. An ultraprocessive, accurate reverse transcriptase encoded by a metazoan group II intron. RNA 24, 183-195 (2018).
Zimmerly, S. & Wu, L. An Unexplored Diversity of Reverse Transcriptases in Bacteria. Microbiol Spectr 3, MDNA3-0058-2014 (2015).
Ostertag, E. M. & Kazazian Jr, H. H. Biology of Mammalian L1 Retrotransposons. Annual Review of Genetics 35, 501-538 (2001).
Perach, M. & Hizi, A. Catalytic Features of the Recombinant Reverse Transcriptase of Bovine Leukemia Virus Expressed in Bacteria. Virology 259, 176-189 (1999).
Lim, D. et al. Crystal structure of the moloney murine leukemia virus RNase H domain. J. Virol. 80, 8379-8389 (2006).
Zhao, C. & Pyle, A. M. Crystal structures of a group II intron maturase reveal a missing link in spliceosome evolution. Nature Structural & Molecular Biology 23, 558-565 (2016).
Griffiths, D. J. Endogenous retroviruses in the human genome sequence. Genome Biol. 2, REVIEWS1017 (2001).
Baranauskas, A. et al. Generation and characterization of new highly thermostable and processive M-MuLV reverse transcriptase variants. Protein Eng Des Sel 25, 657-668 (2012).
Zimmerly, S., Guo, H., Perlman, P. S. & Lambowltz, A. M. Group II intron mobility occurs by target DNA-primed reverse transcription. Cell 82, 545-554 (1995).
Feng, Q., Moran, J. V., Kazazian, H. H. & Boeke, J. D. Human L1 retrotransposon encodes a conserved endonuclease required for retrotransposition. Cell 87, 905-916 (1996).
Berkhout, B., Jebbink, M. & Zsiros, J. Identification of an Active Reverse Transcriptase Enzyme Encoded by a Human Endogenous HERV-K Retrovirus. Journal of Virology 73, 2365-2375 (1999).
Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).
Arezi, B. & Hogrefe, H. Novel mutations in Moloney Murine Leukemia Virus reverse transcriptase increase thermostability through tighter binding to template-primer. Nucleic Acids Res 37, 473-481 (2009).
Blain, S. W. & Goff, S. P. Nuclease activities of Moloney murine leukemia virus reverse transcriptase. Mutants with altered substrate specificities. J. Biol. Chem. 268, 23585-23592 (1993).
Xiong, Y. & Eickbush, T. H. Origin and evolution of retroelements based upon their reverse transcriptase sequences. EMBO J 9, 3353-3362 (1990).
Herschhorn, A. & Hizi, A. Retroviral reverse transcriptases. Cell. Mol. Life Sci. 67, 2717-2747 (2010).
Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).
Liu, M. et al. Reverse Transcriptase-Mediated Tropism Switching in Bordetella Bacteriophage. Science 295, 2091-2094 (2002).
Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
Nottingham, R. M. et al. RNA-seq of human reference RNA samples using a thermostable group II intron reverse transcriptase. RNA 22, 597-613 (2016).
Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).
Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).
Nowak, E. et al. Structural analysis of monomeric retroviral reverse transcriptase in complex with an RNA/DNA hybrid. Nucleic Acids Res 41, 3874-3887 (2013).
Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).
Das, D. & Georgiadis, M. M. The Crystal Structure of the Monomeric Reverse Transcriptase from Moloney Murine Leukemia Virus. Structure 12, 819-829 (2004).
Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).
Gerard, G. F. et al. The role of template-primer in protection of reverse transcriptase from thermal inactivation. Nucleic Acids Res 30, 3118-3129 (2002).
Monot, C. et al. The Specificity and Flexibility of L1 Reverse Transcription Priming at Imperfect T-Tracts. PLOS Genetics 9, e1003499 (2013).
Mohr, S. et al. Thermostable group II intron reverse transcriptase fusion proteins and their use in cDNA synthesis and next-generation RNA sequencing. RNA 19, 958-970 (2013).

Any of the references noted above which relate to reverse transcriptases are hereby incorporated by reference in their entireties, if not already stated so.

### [4] Prime Editors

The prime editor (PE) system described herein refers to a system comprising (A) at least two proteins: (1) a napDNAbp (e.g., a Cas9 nickase) and (2) a polymerase (e.g., DNA-dependent DNA polymerase or RNA-dependent DNA polymerase, such as, reverse transcriptase) and (B) an engineered pegRNA comprising at least one performance-enhancing modification relative to a canonical pegRNA. The napDNAbp and the polymerase components may be provided separately, i.e., in trans to one another, or may be provided as a fusion protein whereby the napDNAbp and polymerase components are coupled, e.g., via a polypeptide linker.

The application contemplates any suitable napDNAbp and polymerase (e.g., DNA-dependent DNA polymerase or RNA-dependent DNA polymerase, such as, reverse transcriptase) to be combined in a single fusion protein for use with the herein disclosed engineered pegRNAs. Examples of napDNAbps and polymerases (e.g., DNA-dependent DNA polymerase or RNA-dependent DNA polymerase, such as, reverse transcriptase) are each defined herein. Since polymerases are well-known in the art, and the amino acid sequences are readily available, this disclosure is not meant in any way to be limited to those specific polymerases identified herein.

In various embodiments, the fusion proteins may comprise any suitable structural configuration. For example, the fusion protein may comprise from the N-terminus to the C-terminus direction, a napDNAbp fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂—[RT]-[napDNAbp]-COOH, wherein each instance of “]-[” indicates the presence of an optional linker sequence.

An exemplary fusion protein is depicted in FIG. 14, which shows a fusion protein comprising an MLV reverse transcriptase (“MLV-RT”) fused to a nickase Cas9 (“Cas9(H840A)”) via a linker sequence. This example is not intended to limit scope of fusion proteins that may be utilized for the prime editor (PE) system described herein.

In various embodiments, the prime editor may have the following amino acid sequence (referred to herein as “PE1”), which includes a Cas9 variant comprising an H840A mutation (i.e., a Cas9 nickase) and an M-MLV RT wild type, as well as an N-terminal NLS sequence (19 amino acids) and an amino acid linker (32 amino acids) that joins the C-terminus of the Cas9 nickase domain to the N-terminus of the RT domain. The PE1 fusion protein has the following structure: [NLS]-[Cas9(H840A)]-[linker]-[MMLV_RT(wt)]. The amino acid sequence of PE1 and its individual components are as follows:

| DESCRIPTION | SEQUENCE |
|---|---|
| PE1 FUSION | MKRTADGSEFESPKKKRKVDKKYSIGLDIGTNSVGWAVITDEYKVPSK |
| PROTEIN | KFKVLGNTDRHSIKKNLIGALLFDSGETAEATRLKRTARRRYTRRK |
| CAS9(H840A)- | NRICYLQEIFSNEMAKVDDSFFHRLEESFLVEEDKKHERHPIFGNIVD |
| MMLV_RT(WT) | EVAYHEKYPTIYHLRKKLVDSTDKADLRLIYLALAHMIKFRGHFLIE |
| | GDLNPDNSDVDKLFIQLVQTYNQLFEENPINASGVDAKAILSARLSKS |
| | RRLENLIAQLPGEKKNGLFGNLIALSLGLTPNFKSNFDLAEDAKLQL |
| | SKDTYDDDLDNLLAQIGDQYADLFLAAKNLSDAILLSDILRVNTEITK |
| | APLSASMIKRYDEHHQDLTLLKALVRQQLPEKYKEIFFDQSKNGYA |
| | GYIDGGASQEEFYKFIKPILEKMDGTEELLVKLNREDLLRKQRTFDN |
| | GSIPHQIHLGELHAILRRQEDFYPFLKDNREKIEKILTFRIPYYVGPL |
| | ARGNSRFAWMTRKSEETITPWNFEEVVDKGASAQSFIERMTNFDKN |
| | LPNEKVLPKHSLLYEYFTVYNELTKVKYVTEGMRKPAFLSGEQKKA |
| | IVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHL |
| | FDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKK |
| | GILQTVKVVDELVKVMGRHKPENIVIEMARENQTTQKGQKNSRER |
| | MKRIEEGIKELGSQILKEHPVENTQLQNEKLYLYYLQNGRDMYVDQ |
| | ELDINRLSDYDVDAIVPQSFLKDDSIDNKVLTRSDKNRGKSDNVPSEE |
| | VVKKMKNYWRQLLNAKLITQRKFDNLTKAERGGLSELDKAGFIKR |
| | QLVETRQITKHVAQILDSRMNTKYDENDKLIREVKVITLKSKLVSDF |
| | RKDFQFYKVREINNYHHAHDAYLNAVVGTALIKKYPKLESEFVYGD |
| | YKVYDVRKMIAKSEQEIGKATAKYFFYSNIMNFFKTEITLANGEIRK |
| | RPLIETNGETGEIVWDKGRDFATVRKVLSMPQVNIVKKTEVQTGGF |
| | SKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPK |
| | YSLFELENGRKRMLASAGELQKGNELALPSKYVNFLYLASHYEKLK |
| | GSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEV |
| | LDATLIHQSITGLYETRIDLSQLGGDSGGSSGGSSGSETPGTSESATPE |
| | SSGGSSGGSSTLNIEDEYRLHETSKEPDVSLGSTWLSDFPQAWAETGGMGL |
| | AVRQAPLIIPLKATSTPVSIKQYPMSQEARLGIKPHIQRLLDQGILVPCQSPWN |
| | TPLLPVKKPGTNDYRPVQDLREVNKRVEDIHPTVPNPYNLLSGLPPSHQWYT |
| | VLDLKDAFFCLRLHPTSQPLFAFEWRDPEMGISGQLTWTRLPQGFKNSPTL |
| | FDEALHRDLADFRIQHPDLILLQYVDDLLLAATSELDCQQGTRALLQTLGNL |
| | GYRASAKKAQICQKQVKYLGYLLKEGQRWLTEARKETVMGQPTPKTPRQLR |
| | EFLGTAGFCRLWIPGFAEMAAPLYPLTKTGTLFNWGPDQQKAYQEIKQALLT |
| | APALGLPDLTKPFELFVDEKQGYAKGVLTQKLGPWRRPVAYLSKKLDPVAAG |
| | WPPCLRMVAAIAVLTKDAGKLTMGQPLVILAPHAVEALVKQPPDRWLSNAR |
| | MTHYQALLLDTDRVQFGPVVALNPATLLPLPEEGLQHNCLDILAEAHGTRPD |
| | LTDQPLPDADHTWYTDGSSLLQEGQRKAGAAVTTETEVIWAKALPAGTSAQR |
| | AELIALTQALKMAEGKKLNVYTDSRYAFATAHIHGEIYRRRGLLTSEGKEIKNK |
| | DEILALLKALFLPKRLSIIHCPGHQKGHSAEARGNRMADQAARKAAITETPD |
| | TSTLLIENSSPSGGSKRTADGSEFEPKKKRKV (SEQ ID NO: 28) |
| | KEY: |
| | NUCLEAR LOCALIZATION SEQUENCE (NLS) TOP: (SEQ ID NO: 29), |
| | BOTTOM: (SEQ ID NO: 30) |
| | CAS9(H840A)(SEQ ID NO: 31) |
| | 33-AMINO ACID LINKER (SEQ ID NO: 11) |
| | M-MLV REVERSE TRANSCRIPTASE (SEQ ID NO: 32) |
| PE1 - N- | MKRTADGSEFESPKKKRKV (SEQ ID NO: 29) |
| TERMINAL | |
| NLS | |
| PE1 - CAS9 | DKKYSIGLDIGTNSVGWAVITDEYKVPSKKFKVLGNTDRHSIKKNLIGAL |
| (H840A)(MET | LFDSGETAEATRLKRTARRRYTRRKNRICYLQEIFSNEMAKVDDSFFHRL |
| MINUS) | EESFLVEEDKKHERHPIFGNIVDEVAYHEKYPTIYHLRKKLVDSTDKADL |
| | RLIYLALAHMIKFRGHFLIEGDLNPDNSDVDKLFIQLVQTYNQLFEENPI |
| | NASGVDAKAILSARLSKSRRLENLIAQLPGEKKNGLFGNLIALSLGLTPN |
| | FKSNFDLAEDAKLQLSKDTYDDDLDNLLAQIGDQYADLFLAAKNLSDAI |
| | LLSDILRVNTEITKAPLSASMIKRYDEHHQDLTLLKALVRQQLPEKYKEIF |
| | FDQSKNGYAGYIDGGASQEEFYKFIKPILEKMDGTEELLVKLNREDLLR |
| | KQRTFDNGSIPHQIHLGELHAILRRQEDFYPFLKDNREKIEKILTFRIPYYV |
| | GPLARGNSRFAWMTRKSEETITPWNFEEVVDKGASAQSFIERMTNFDKN |
| | LPNEKVLPKHSLLYEYFTVYNELTKVKYVTEGMRKPAFLSGEQKKAIVD |
| | LLFKTNRKVTVKQLKEDYFKKIECFDSVEISGVEDRFNASLGTYHDLLKI |
| | IKDKDFLDNEENEDILEDIVLTLTLFEDREMIEERLKTYAHLFDDKVMKQ |
| | LKRRRYTGWGRLSRKLINGIRDKQSGKTILDFLKSDGFANRNFMQLIHD |
| | DSLTFKEDIQKAQVSGQGDSLHEHIANLAGSPAIKKGILQTVKVVDELVK |
| | VMGRHKPENIVIEMARENQTTQKGQKNSRERMKRIEEGIKELGSQILKE |
| | HPVENTQLQNEKLYLYYLQNGRDMYVDQELDINRLSDYDVDAIVPQSF |
| | LKDDSIDNKVLTRSDKNRGKSDNVPSEEVVKKMKNYWRQLLNAKLITQ |
| | RKFDNLTKAERGGLSELDKAGFIKRQLVETRQITKHVAQILDSRMNTKY |
| | DENDKLIREVKVITLKSKLVSDFRKDFQFYKVREINNYHHAHDAYLNAV |
| | VGTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMPQ |
| | VNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEV |
| | KKDLIIKLPKYSLFELENGRKRMLASAGELQKGNELALPSKYVNFLYLA |
| | SHYEKLKGSPEDNEQKQLFVEQHKHYLDEIIEQISEFSKRVILADANLDK |
| | VLSAYNKHRDKPIREQAENIIHLFTLTNLGAPAAFKYFDTTIDRKRYTSTK |
| | EVLDATLIHQSITGLYETRIDLSQLGGD (SEQ ID NO: 130) |
| PE1 - LINKER | SGGSSGGSSGSETPGTSESATPESSGGSSGGSS (SEQ ID NO: 11) |
| BETWEEN | |
| CAS9 | |
| DOMAIN AND | |
| RT DOMAIN | |
| (33 AMINO | |
| ACIDS) | |
| PE1 - M-MLV | TLNIEDEYRLHETSKEPDVSLGSTWLSDFPQAWAETGGMGLAVRQAPLII |
| RT | PLKATSTPVSIKQYPMSQEARLGIKPHIQRLLDQGILVPCQSPWNTPLLPV |
| | KKPGTNDYRPVQDLREVNKRVEDIHPTVPNPYNLLSGLPPSHQWYTVL |
| | DLKDAFFCLRLHPTSQPLFAFEWRDPEMGISGQLTWTRLPQGFKNSPTLF |
| | DEALHRDLADFRIQHPDLILLQYVDDLLLAATSELDCQQGTRALLQTLG |
| | NLGYRASAKKAQICQKQVKYLGYLLKEGQRWLTEARKETVMGQPTPK |
| | TPRQLREFLGTAGFCRLWIPGFAEMAAPLYPLTKTGTLFNWGPDQQKAY |
| | QEIKQALLTAPALGLPDLTKPFELFVDEKQGYAKGVLTQKLGPWRRPVA |
| | YLSKKLDPVAAGWPPCLRMVAAIAVLTKDAGKLTMGQPLVILAPHAVEA |
| | LVKQPPDRWLSNARMTHYQALLLDTDRVQFGPVVALNPATLLPLPEEGL |
| | QHNCLDILAEAHGTRPDLTDQPLPDADHTWYTDGSSLLQEGQRKAGAA |
| | VTTETEVIWAKALPAGTSAQRAELIALTQALKMAEGKKLNVYTDSRYAF |
| | ATAHIHGEIYRRRGLLTSEGKEIKNKDEILALLKALFLPKRLSIIHCPGHQK |
| | GHSAEARGNRMADQAARKAAITETPDTSTLLIENSSP (SEQ ID NO: 132) |
| PE1 - C- | SGGSKRTADGSEFEPKKKRKV (SEQ ID NO: 30) |
| TERMINAL | |
| NLS | |

In another embodiment, the prime editor may have the following amino acid sequence (referred to herein as “PE2”), which includes a Cas9 variant comprising an H840A mutation (i.e., a Cas9 nickase) and an M-MLV RT comprising mutations D200N, T330P, L603W, T306K, and W313F, as well as an N-terminal NLS sequence (19 amino acids) and an amino acid linker (33 amino acids) that joins the C-terminus of the Cas9 nickase domain to the N-terminus of the RT domain. The PE2 fusion protein has the following structure: [NLS]-[Cas9(HS40A)]-[linker]-[MMLV-RT(D200N)(T330P)(L603W)(T306K)(W313F)]. The amino acid sequence of PE2 is as follows:

| | |
|---|---|
| PE2 FUSION | MKRTADGSEFESPKKKRKVDKKYSIGLDIGTNSVGWAVITDEYKVPS |
| PROTEIN | KKFKVLGNTDRHSIKKNLIGALLFDSGETAEATRLKRTARRRYTRR |
| CAS9(H840A)- | KNRICYLQEIFSNEMAKVDDSFFHRLEESFLVEEDKKHERHPIFGNI |
| MMLV_RT | VDEVAYHEKYPTIYHLRKKLVDSTDKADLRLIYLALAHMIKFRGHF |
| D200N | LIEGDLNPDNSDVDKLFIQLVQTYNQLFEENPINASGVDAKAILSAR |
| T330P | LSKSRRLENLIAQLPGEKKNGLFGNLIALSLGLTPNFKSNFDLAEDA |
| L603W | KLQLSKDTYDDDLDNLLAQIGDQYADLFLAAKNLSDAILLSDILRV |
| T306K | NTEITKAPLSASMIKRYDEHHQDLTLLKALVRQQLPEKYKEIFFDQS |
| W313F | KNGYAGYIDGGASQEEFYKFIKPILEKMDGTEELLVKLNREDLLRK |
| | QRTFDNGSIPHQIHLGELHAILRRQEDFYPFLKDNREKIEKILTFRIP |
| | YYVGPLARGNSRFAWMTRKSEETITPWNFEEVVDKGASAQSFIER |
| | MTNFDKNLPNEKVLPKHSLLYEYFTVYNELTKVKYVTEGMRKPAF |
| | LSGEQKKAIVDLLFKTNRKVTVKQLKEDYFKKIECFDSVEISGVED |
| | RFNASLGTYHDLLKIIKDKDFLDNEENEDILEDIVLTLTLFEDREMIE |
| | ERLKTYAHLFDDKVMKQLKRRRYTGWGRLSRKLINGIRDKQSGK |
| | TILDFLKSDGFANRNFMQLIHDDSLTFKEDIQKAQVSGQGDSLHEHI |
| | ANLAGSPAIKKGILQTVKVVDELVKVMGRHKPENIVIEMARENQTT |
| | QKGQKNSRERMKRIEEGIKELGSQILKEHPVENTQLQNEKLYLYYL |
| | QNGRDMYVDQELDINRLSDYDVDAIVPQSFLKDDSIDNKVLTRSDK |
| | NRGKSDNVPSEEVVKKMKNYWRQLLNAKLITQRKFDNLTKAERG |
| | GLSELDKAGFIKRQLVETRQITKHVAQILDSRMNTKYDENDKLIRE |
| | VKVITLKSKLVSDFRKDFQFYKVREINNYHHAHDAYLNAVVGTALI |
| | KKYPKLESEFVYGDYKVYDVRKMIAKSEQEIGKATAKYFFYSNIMN |
| | FFKTEITLANGEIRKRPLIETNGETGEIVWDKGRDFATVRKVLSMP |
| | QVNIVKKTEVQTGGFSKESILPKRNSDKLIARKKDWDPKKYGGFDS |
| | PTVAYSVLVVAKVEKGKSKKLKSVKELLGITIMERSSFEKNPIDFLE |
| | AKGYKEVKKDLIIKLPKYSLFELENGRKRMLASAGELQKGNELAL |
| | PSKYVNFLYLASHYEKLKGSPEDNEQKQLFVEQHKHYLDEIIEQISE |
| | FSKRVILADANLDKVLSAYNKHRDKPIREQAENIIHLFTLTNLGAPA |
| | AFKYFDTTIDRKRYTSTKEVLDATLIHQSITGLYETRIDLSQLGGDS |
| | GGSSGGSSGSETPGTSESATPESSGGSSGGSSTLNIEDEYRLHETSKEPD |
| | VSLGSTWLSDFPQAWAETGGMGLAVRQAPLIIPLKATSTPVSIKQYPMSQEA |
| | RLGIKPHIQRLLDQGILVPCQSPWNTPLLPVKKPGTNDYRPVQDLREVNKR |
| | VEDIHPTVPNPYNLLSGLPPSHQWYTVLDLKDAFFCLRLHPTSQPLFAFEW |
| | RDPEMGISGQLTWTRLPQGFKNSPTLFNEALHRDLADFRIQHPDLILLQYV |
| | DDLLLAATSELDCQQGTRALLQTLGNLGYRASAKKAQICQKQVKYLGYLLK |
| | EGQRWLTEARKETVMGQPTPKTPRQLREFLGKAGFCRLFIPGFAEMAAPLY |
| | PLTKPGTLFNWGPDQQKAYQEIKQALLTAPALGLPDLTKPFELFVDEKQGY |
| | AKGVLTQKLGPWRRPVAYLSKKLDPVAAGWPPCLRMVAAIAVLTKDAGKLT |
| | MGQPLVILAPHAVEALVKQPPDRWLSNARMTHYQALLLDTDRVQFGPVVA |
| | LNPATLLPLPEEGLQHNCLDILAEAHGTRPDLTDQPLPDADHTWYTDGSSL |
| | LQEGQRKAGAAVTTETEVIWAKALPAGTSAQRAELIALTQALKMAEGKKLNV |
| | YTDSRYAFATAHIHGEIYRRRGWLTSEGKEIKNKDEILALLKALFLPKRLSIIH |
| | CPGHQKGHSAEARGNRMADQAARKAAITETPDTSTLLIENSSPSGGSKRTA |
| | DGSEFEPKKKRKV (SEQ ID NO: 33) |
| | KEY: |
| | NUCLEAR LOCALIZATION SEQUENCE (NLS) TOP: (SEQ ID NO: 29), |
| | BOTTOM: (SEQ ID NO: 30) |
| | CAS9(H840A)(SEQ ID NO: 31) |
| | 33-AMINO ACID LINKER (SEQ ID NO: 11) |
| | M-MLV REVERSE TRANSCRIPTASE (SEQ ID NO: 34) |
| PE2 - N- | MKRTADGSEFESPKKKRKV (SEQ ID NO: 29) |
| TERMINAL | |
| NLS | |
| PE2 - CAS9 | DKKYSIGLDIGTNSVGWAVITDEYKVPSKKFKVLGNTDRHSIKKNLIGA |
| (H840A)(MET | LLFDSGETAEATRLKRTARRRYTRRKNRICYLQEIFSNEMAKVDDSFFH |
| MINUS) | RLEESFLVEEDKKHERHPIFGNIVDEVAYHEKYPTIYHLRKKLVDSTDKA |
| | DLRLIYLALAHMIKFRGHFLIEGDLNPDNSDVDKLFIQLVQTYNQLFEE |
| | NPINASGVDAKAILSARLSKSRRLENLIAQLPGEKKNGLFGNLIALSLGL |
| | TPNFKSNFDLAEDAKLQLSKDTYDDDLDNLLAQIGDQYADLFLAAKNL |
| | SDAILLSDILRVNTEITKAPLSASMIKRYDEHHQDLTLLKALVRQQLPEK |
| | YKEIFFDQSKNGYAGYIDGGASQEEFYKFIKPILEKMDGTEELLVKLNR |
| | EDLLRKQRTFDNGSIPHQIHLGELHAILRRQEDFYPFLKDNREKIEKILTF |
| | RIPYYVGPLARGNSRFAWMTRKSEETITPWNFEEVVDKGASAQSFIERM |
| | TNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLG |
| | TYHDLLKIIKDKDFLDNEENEDILEDIVLTLTLFEDREMIEERLKTYAHLF |
| | DDKVMKQLKRRRYTGWGRLSRKLINGIRDKQSGKTILDFLKSDGFANR |
| | NFMQLIHDDSLTFKEDIQKAQVSGQGDSLHEHIANLAGSPAIKKGILQT |
| | VKVVDELVKVMGRHKPENIVIEMARENQTTQKGQKNSRERMKRIEEGI |
| | KELGSQILKEHPVENTQLQNEKLYLYYLQNGRDMYVDQELDINRLSDY |
| | DVDAIVPQSFLKDDSIDNKVLTRSDKNRGKSDNVPSEEVVKKMKNYW |
| | RQLLNAKLITQRKFDNLTKAERGGLSELDKAGFIKRQLVETRQITKHVA |
| | QILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEI |
| | GKATAKYFFYSNIMNFFKTEITLANGEIRKRPLIETNGETGEIVWDKGRD |
| | FATVRKVLSMPQVNIVKKTEVQTGGFSKESILPKRNSDKLIARKKDWDP |
| | KKYGGFDSPTVAYSVLVVAKVEKGKSKKLKSVKELLGITIMERSSFEKN |
| | PIDFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQISEF |
| | SKRVILADANLDKVLSAYNKHRDKPIREQAENIIHLFTLTNLGAPAAFKY |
| | FDTTIDRKRYTSTKEVLDATLIHQSITGLYETRIDLSQLGGD (SEQ ID NO: |
| | 31) |
| PE2 - LINKER | SGGSSGGSSGSETPGTSESATPESSGGSSGGSS (SEQ ID NO: 11) |
| BETWEEN | |
| CAS9 | |
| DOMAIN | |
| AND RT | |
| DOMAIN (33 | |
| AMINO | |
| ACIDS) | |
| PE2 | TLNIEDEYRLHETSKEPDVSLGSTWLSDFPQAWAETGGMGLAVRQAPLI |
| MMLV_RT | IPLKATSTPVSIKQYPMSQEARLGIKPHIQRLLDQGILVPCQSPWNTPLLP |
| D200N | VKKPGTNDYRPVQDLREVNKRVEDIHPTVPNPYNLLSGLPPSHQWYTV |
| T330P | LDLKDAFFCLRLHPTSQPLFAFEWRDPEMGISGQLTWTRLPQGFKNSPT |
| L603W | LFNEALHRDLADFRIQHPDLILLQYVDDLLLAATSELDCQQGTRALLQT |
| T306K | LGNLGYRASAKKAQICQKQVKYLGYLLKEGQRWLTEARKETVMGQPT |
| W313F | PKTPRQLREFLGKAGFCRLFIPGFAEMAAPLYPLTKPGTLFNWGPDQQK |
| | AYQEIKQALLTAPALGLPDLTKPFELFVDEKQGYAKGVLTQKLGPWRRP |
| | VAYLSKKLDPVAAGWPPCLRMVAAIAVLTKDAGKLTMGQPLVILAPHA |
| | VEALVKQPPDRWLSNARMTHYQALLLDTDRVQFGPVVALNPATLLPLP |
| | EEGLQHNCLDILAEAHGTRPDLTDQPLPDADHTWYTDGSSLLQEGQRK |
| | AGAAVTTETEVIWAKALPAGTSAQRAELIALTQALKMAEGKKLNVYTD |
| | SRYAFATAHIHGEIYRRRGWLTSEGKEIKNKDEILALLKALFLPKRLSIIH |
| | CPGHQKGHSAEARGNRMADQAARKAAITETPDTSTLLIENSSP (SEQ ID |
| | NO: 34) |
| PE2 - C- | SGGSKRTADGSEFEPKKKRKV (SEQ ID NO: 30) |
| TERMINAL | |
| NLS | |

In still other embodiments, the prime editor may have the following amino acid sequences:

| | |
|---|---|
| PRIME | MKRTADGSEFESPKKKRKVTLNIEDEYRLHETSKEPDVSLGSTWLSDFPQA |
| EDITOR | WAETGGMGLAVRQAPLIIPLKATSTPVSIKQYPMSQEARLGIKPHIQRLLDQG |
| MMLV_RT(WT)- | ILVPCQSPWNTPLLPVKKPGTNDYRPVQDLREVNKRVEDIHPTVPNPYNLLS |
| 32AA- | GLPPSHQWYTVLDLKDAFFCLRLHPTSQPLFAFEWRDPEMGISGQLTWTRL |
| CAS9(H840A) | PQGFKNSPTLFDEALHRDLADFRIQHPDLILLQYVDDLLLAATSELDCQQGT |
| | RALLQTLGNLGYRASAKKAQICQKQVKYLGYLLKEGQRWLTEARKETVMGQ |
| | PTPKTPRQLREFLGTAGFCRLWIPGFAEMAAPLYPLTKTGTLFNWGPDQQK |
| | AYQEIKQALLTAPALGLPDLTKPFELFVDEKQGYAKGVLTQKLGPWRRPVAY |
| | LSKKLDPVAAGWPPCLRMVAAIAVLTKDAGKLTMGQPLVILAPHAVEALVKQ |
| | PPDRWLSNARMTHYQALLLDTDRVQFGPVVALNPATLLPLPEEGLQHNCLDI |
| | LAEAHGTRPDLTDQPLPDADHTWYTDGSSLLQEGQRKAGAAVTTETEVIWA |
| | KALPAGTSAQRAELIALTQALKMAEGKKLNVYTDSRYAFATAHIHGEIYRRRGL |
| | LTSEGKEIKNKDEILALLKALFLPKRLSIIHCPGHQKGHSAEARGNRMADQA |
| | ARKAAITETPDTSTLLIENSSPSGGSSGGSSGSETPGTSESATPESSGGSSG |
| | GSSDKKYSIGLDIGTNSVGWAVITDEYKVPSKKFKVLGNTDRHSIKK |
| | NLIGALLFDSGETAEATRLKRTARRRYTRRKNRICYLQEIFSNEMAK |
| | VDDSFFHRLEESFLVEEDKKHERHPIFGNIVDEVAYHEKYPTIYHLRK |
| | KLVDSTDKADLRLIYLALAHMIKFRGHFLIEGDLNPDNSDVDKLFIQ |
| | LVQTYNQLFEENPINASGVDAKAILSARLSKSRRLENLIAQLPGEKK |
| | NGLFGNLIALSLGLTPNFKSNFDLAEDAKLQLSKDTYDDDLDNLLAQ |
| | IGDQYADLFLAAKNLSDAILLSDILRVNTEITKAPLSASMIKRYDEHH |
| | QDLTLLKALVRQQLPEKYKEIFFDQSKNGYAGYIDGGASQEEFYKFI |
| | KPILEKMDGTEELLVKLNREDLLRKQRTFDNGSIPHQIHLGELHAIL |
| | RRQEDFYPFLKDNREKIEKILTFRIPYYVGPLARGNSRFAWMTRKSE |
| | ETITPWNFEEVVDKGASAQSFIERMTNFDKNLPNEKVLPKHSLLYEY |
| | FTVYNELTKVKYVTEGMRKPAFLSGEQKKAIVDLLFKTNRKVTVK |
| | QLKEDYFKKIECFDSVEISGVEDRFNASLGTYHDLLKIIKDKDFLDN |
| | EENEDILEDIVLTLTLFEDREMIEERLKTYAHLFDDKVMKQLKRRRY |
| | TGWGRLSRKLINGIRDKQSGKTILDFLKSDGFANRNFMQLIHDDSLT |
| | FKEDIQKAQVSGQGDSLHEHIANLAGSPAIKKGILQTVKVVDELVKV |
| | MGRHKPENIVIEMARENQTTQKGQKNSRERMKRIEEGIKELGSQIL |
| | KEHPVENTQLQNEKLYLYYLQNGRDMYVDQELDINRLSDYDVDAIV |
| | PQSFLKDDSIDNKVLTRSDKNRGKSDNVPSEEVVKKMKNYWRQLLN |
| | AKLITQRKFDNLTKAERGGLSELDKAGFIKRQLVETRQITKHVAQIL |
| | DSRMNTKYDENDKLIREVKVITLKSKLVSDFRKDFQFYKVREINNYH |
| | HAHDAYLNAVVGTALIKKYPKLESEFVYGDYKVYDVRKMIAKSEQE |
| | IGKATAKYFFYSNIMNFFKTEITLANGEIRKRPLIETNGETGEIVWDK |
| | GRDFATVRKVLSMPQVNIVKKTEVQTGGFSKESILPKRNSDKLIARK |
| | KDWDPKKYGGFDSPTVAYSVLVVAKVEKGKSKKLKSVKELLGITIM |
| | ERSSFEKNPIDFLEAKGYKEVKKDLIIKLPKYSLFELENGRKRMLAS |
| | AGELQKGNELALPSKYVNFLYLASHYEKLKGSPEDNEQKQLFVEQH |
| | KHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETR |
| | IDLSQLGGDSGGSKRTADGSEFEPKKKRKV (SEQ ID NO: 129) |
| | KEY: |
| | NUCLEAR LOCALIZATION SEQUENCE (NLS) TOP: (SEQ ID NO: 29), |
| | BOTTOM: (SEQ ID NO: 30) |
| | CAS9(H840A) (SEQ ID NO: 31) |
| | 33-AMINO ACID LINKER (SEQ ID NO: 11) |
| | M-MLV REVERSE TRANSCRIPTASE (SEQ ID NO: 32) |
| PRIME | MKRTADGSEFESPKKKRKVTLNIEDEYRLHETSKEPDVSLGSTWLSDFPQA |
| EDITOR | WAETGGMGLAVRQAPLIIPLKATSTPVSIKQYPMSQEARLGIKPHIQRLLDQG |
| MMLV_RT(WT)- | ILVPCQSPWNTPLLPVKKPGTNDYRPVQDLREVNKRVEDIHPTVPNPYNLLS |
| 60AA- | GLPPSHQWYTVLDLKDAFFCLRLHPTSQPLFAFEWRDPEMGISGQLTWTRL |
| CAS9(H840A) | PQGFKNSPTLFDEALHRDLADFRIQHPDLILLQYVDDLLLAATSELDCQQGT |
| | RALLQTLGNLGYRASAKKAQICQKQVKYLGYLLKEGQRWLTEARKETVMGQ |
| | PTPKTPRQLREFLGTAGFCRLWIPGFAEMAAPLYPLTKTGTLFNWGPDQQK |
| | AYQEIKQALLTAPALGLPDLTKPFELFVDEKQGYAKGVLTQKLGPWRRPVAY |
| | LSKKLDPVAAGWPPCLRMVAAIAVLTKDAGKLTMGQPLVILAPHAVEALVKQ |
| | PPDRWLSNARMTHYQALLLDTDRVQFGPVVALNPATLLPLPEEGLQHNCLDI |
| | LAEAHGTRPDLTDQPLPDADHTWYTDGSSLLQEGQRKAGAAVTTETEVIWA |
| | KALPAGTSAQRAELIALTQALKMAEGKKLNVYTDSRYAFATAHIHGEIYRRRGL |
| | LTSEGKEIKNKDEILALLKALFLPKRLSIIHCPGHQKGHSAEARGNRMADQA |
| | ARKAAITETPDTSTLLIENSSPSGGSSGGSSGSETPGTSESATPESAGSYPY |
| | DVPDYAGSAAPAAKKKKLDGSGSGGSSGGSDKKYSIGLDIGTNSVG |
| | WAVITDEYKVPSKKFKVLGNTDRHSIKKNLIGALLFDSGETAEATRL |
| | KRTARRRYTRRKNRICYLQEIFSNEMAKVDDSFFHRLEESFLVEEDK |
| | KHERHPIFGNIVDEVAYHEKYPTIYHLRKKLVDSTDKADLRLIYLAL |
| | AHMIKFRGHFLIEGDLNPDNSDVDKLFIQLVQTYNQLFEENPINASG |
| | VDAKAILSARLSKSRRLENLIAQLPGEKKNGLFGNLIALSLGLTPNFK |
| | SNFDLAEDAKLQLSKDTYDDDLDNLLAQIGDQYADLFLAAKNLSDAI |
| | LLSDILRVNTEITKAPLSASMIKRYDEHHQDLTLLKALVRQQLPEKY |
| | KEIFFDQSKNGYAGYIDGGASQEEFYKFIKPILEKMDGTEELLVKLN |
| | REDLLRKQRTFDNGSIPHQIHLGELHAILRRQEDFYPFLKDNREKIE |
| | KILTFRIPYYVGPLARGNSRFAWMTRKSEETITPWNFEEVVDKGASA |
| | QSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEIS |
| | GVEDRFNASLGTYHDLLKIIKDKDFLDNEENEDILEDIVLTLTLFEDR |
| | EMIEERLKTYAHLFDDKVMKQLKRRRYTGWGRLSRKLINGIRDKQ |
| | SGKTILDFLKSDGFANRNFMQLIHDDSLTFKEDIQKAQVSGQGDSLH |
| | EHIANLAGSPAIKKGILQTVKVVDELVKVMGRHKPENIVIEMAREN |
| | QTTQKGQKNSRERMKRIEEGIKELGSQILKEHPVENTQLQNEKLYL |
| | YYLQNGRDMYVDQELDINRLSDYDVDAIVPQSFLKDDSIDNKVLTRS |
| | DKNRGKSDNVPSEEVVKKMKNYWRQLLNAKLITQRKFDNLTKAER |
| | GGLSELDKAGFIKRQLVETRQITKHVAQILDSRMNTKYDENDKLIRE |
| | VKVITLKSKLVSDFRKDFQFYKVREINNYHHAHDAYLNAVVGTALIK |
| | KYPKLESEFVYGDYKVYDVRKMIAKSEQEIGKATAKYFFYSNIMNFF |
| | KTEITLANGEIRKRPLIETNGETGEIVWDKGRDFATVRKVLSMPQVN |
| | IVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGY |
| | KEVKKDLIIKLPKYSLFELENGRKRMLASAGELQKGNELALPSKYV |
| | NFLYLASHYEKLKGSPEDNEQKQLFVEQHKHYLDEIIEQISEFSKRVI |
| | LADANLDKVLSAYNKHRDKPIREQAENIIHLFTLTNLGAPAAFKYFD |
| | TTIDRKRYTSTKEVLDATLIHQSITGLYETRIDLSQLGGDSGGSKRTA |
| | DGSEFEPKKKRKV (SEQ ID NO: 130) |
| | KEY: |
| | NUCLEAR LOCALIZATION SEQUENCE (NLS) TOP: (SEQ ID NO: 29), |
| | BOTTOM: (SEQ ID NO: 30) |
| | CAS9(H840A)(SEQ ID NO: 31) |
| | AMINO ACID LINKER (SEQ ID NO: 131) |
| | M-MLV REVERSE TRANSCRIPTASE (SEQ ID NO: 132) |
| PRIME | MKRTADGSEFESPKKKRKVDKKYSIGLDIGTNSVGWAVITDEYKVPSK |
| EDITOR | KFKVLGNTDRHSIKKNLIGALLFDSGETAEATRLKRTARRRYTRRK |
| CAS9(H840A)- | NRICYLQEIFSNEMAKVDDSFFHRLEESFLVEEDKKHERHPIFGNIVD |
| FEN1- | EVAYHEKYPTIYHLRKKLVDSTDKADLRLIYLALAHMIKFRGHFLIE |
| MMLV_RT | GDLNPDNSDVDKLFIQLVQTYNQLFEENPINASGVDAKAILSARLSKS |
| D200N | RRLENLIAQLPGEKKNGLFGNLIALSLGLTPNFKSNFDLAEDAKLQL |
| T330P | SKDTYDDDLDNLLAQIGDQYADLFLAAKNLSDAILLSDILRVNTEITK |
| L603W | APLSASMIKRYDEHHQDLTLLKALVRQQLPEKYKEIFFDQSKNGYA |
| T306K | GYIDGGASQEEFYKFIKPILEKMDGTEELLVKLNREDLLRKQRTFDN |
| W313F | GSIPHQIHLGELHAILRRQEDFYPFLKDNREKIEKILTFRIPYYVGPL |
| | ARGNSRFAWMTRKSEETITPWNFEEVVDKGASAQSFIERMTNFDKN |
| | LPNEKVLPKHSLLYEYFTVYNELTKVKYVTEGMRKPAFLSGEQKKA |
| | IVDLLFKTNRKVTVKQLKEDYFKKIECFDSVEISGVEDRFNASLGTY |
| | HDLLKIIKDKDFLDNEENEDILEDIVLTLTLFEDREMIEERLKTYAHL |
| | FDDKVMKQLKRRRYTGWGRLSRKLINGIRDKQSGKTILDFLKSDGF |
| | ANRNFMQLIHDDSLTFKEDIQKAQVSGQGDSLHEHIANLAGSPAIKK |
| | GILQTVKVVDELVKVMGRHKPENIVIEMARENQTTQKGQKNSRER |
| | MKRIEEGIKELGSQILKEHPVENTQLQNEKLYLYYLQNGRDMYVDQ |
| | ELDINRLSDYDVDAIVPQSFLKDDSIDNKVLTRSDKNRGKSDNVPSEE |
| | VVKKMKNYWRQLLNAKLITQRKFDNLTKAERGGLSELDKAGFIKR |
| | QLVETRQITKHVAQILDSRMNTKYDENDKLIREVKVITLKSKLVSDF |
| | RKDFQFYKVREINNYHHAHDAYLNAVVGTALIKKYPKLESEFVYGD |
| | YKVYDVRKMIAKSEQEIGKATAKYFFYSNIMNFFKTEITLANGEIRK |
| | RPLIETNGETGEIVWDKGRDFATVRKVLSMPQVNIVKKTEVQTGGF |
| | SKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPK |
| | YSLFELENGRKRMLASAGELQKGNELALPSKYVNFLYLASHYEKLK |
| | GSPEDNEQKQLFVEQHKHYLDEIIEQISEFSKRVILADANLDKVLSAY |
| | NKHRDKPIREQAENIIHLFTLTNLGAPAAFKYFDTTIDRKRYTSTKEV |
| | LDATLIHQSITGLYETRIDLSQLGGDSGGSSGGSSGSETPGTSESATPE |
| | SSGGSSGGSSGIQGLAKLIADVAPSAIRENDIKSYFGRKVAIDASMSIYQF |
| | LIAVRQGGDVLQNEEGETTSHLMGMFYRTIRMMENGIKPVYVFDGKPP |
| | QLKSGELAKRSERRAEAEKQLQQAQAAGAEQEVEKFTKRLVKVTKQH |
| | NDECKHLLSLMGIPYLDAPSEAEASCAALVKAGKVYAAATEDMDCLTF |
| | GSPVLMRHLTASEAKKLPIQEFHLSRILQELGLNQEQFVDLCILLGSDYC |
| | ESIRGIGPKRAVDLIQKHKSIEEIVRRLDPNKYPVPENWLHKEAHQLFLEP |
| | EVLDPESVELKWSEPNEEELIKFMCGEKQFSEERIRSGVKRLSKSRQGST |
| | QGRLDDFFKVTGSLSSAKRKEPEPKGSTKKKAKTGAAGKFKRGKSGGS |
| | SGGSSGSETPGTSESATPESSGGSSGGSSTLNIEDEYRLHETSKEPDVSLG |
| | STWLSDFPQAWAETGGMGLAVRQAPLIIPLKATSTPVSIKQYPMSQEARLGIK |
| | PHIQRLLDQGILVPCQSPWNTPLLPVKKPGTNDYRPVQDLREVNKRVEDIHP |
| | TVPNPYNLLSGLPPSHQWYTVLDLKDAFFCLRLHPTSQPLFAFEWRDPEMGI |
| | SGQLTWTRLPQGFKNSPTLFNEALHRDLADFRIQHPDLILLQYVDDLLLAAT |
| | SELDCQQGTRALLQTLGNLGYRASAKKAQICQKQVKYLGYLLKEGQRWLTE |
| | ARKETVMGQPTPKTPRQLREFLGKAGFCRLFIPGFAEMAAPLYPLTKPGTLF |
| | NWGPDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQPLVILAP |
| | HAVEALVKQPPDRWLSNARMTHYQALLLDTDRVQFGPVVALNPATLLPLPEE |
| | GLQHNCLDILAEAHGTRPDLTDQPLPDADHTWYTDGSSLLQEGQRKAGAA |
| | VTTETEVIWAKALPAGTSAQRAELIALTQALKMAEGKKLNVYTDSRYAFATAHI |
| | HGEIYRRRGWLTSEGKEIKNKDEILALLKALFLPKRLSIIHCPGHQKGHSAEA |
| | RGNRMADQAARKAAITETPDTSTLLIENSSPSGGSKRTADGSEFEPKKKRKV |
| | (SEQ ID NO: 133) |
| | KEY: |
| | NUCLEAR LOCALIZATION SEQUENCE (NLS) TOP: (SEQ ID NO: 29), |
| | BOTTOM: (SEQ ID NO: 30) |
| | CAS9(H840A)(SEQ ID NO: 31) |
| | 33-AMINO ACID LINKER 1 (SEQ ID NO: 11) |
| | M-MLV REVERSE TRANSCRIPTASE (SEQ ID NO: 34) |
| | 33-AMINO ACID LINKER 2 (SEQ ID NO: 11) |
| | FEN1 (SEQ ID NO: 134) |

In other embodiments, the prime editor can be based on SaCas9 or on SpCas9 nickases with altered PAM specificities, such as the following exemplary sequences:

| | | |
|---|---|---|
| SACAS9-M- | MKRTADGSEFESPKKKRKVGKRNYILGLDIGITSVGYGIIDY | SEQ ID NO: |
| MLV RT | ETRDVIDAGVRLFKEANVENNEGRRSKRGARRLKRRRRHR | 135 |
| PRIME | IQRVKKLLFDYNLLTDHSELSGINPYEARVKGLSQKLSEEEF | |
| EDITOR | SAALLHLAKRRGVHNVNEVEEDTGNELSTKEQISRNSKAL | |
| | EEKYVAELQLERLKKDGEVRGSINRFKTSDYVKEAKQLLK | |
| | VQKAYHQLDQSFIDTYIDLLETRRTYYEGPGEGSPFGWKDI | |
| | KEWYEMLMGHCTYFPEELRSVKYAYNADLYNALNDLNNL | |
| | VITRDENEKLEYYEKFQUIENVFKQKKKPTLKQIAKEILVNE | |
| | EDIKGYRVTSTGKPEFTNLKVYHDIKDITARKEIIENAELLD | |
| | QIAKILTIYQSSEDIQEELTNLNSELTQEEIEQISNLKGYTGTH | |
| | NLSLKAINLILDELWHTNDNQIAIFNRLKLVPKKVDLSQQKE | |
| | IPTTLVDDFILSPVVKRSFIQSIKVINAIIKKYGLPNDIIIELARE | |
| | KNSKDAQKMINEMQKRNRQTNERIEEIIRTTGKENAKYLIE | |
| | KIKLHDMQEGKCLYSLEAIPLEDLLNNPFNYEVDHIIPRSVS | |
| | FDNSFNNKVLVKQEEASKKGNRTPFQYLSSSDSKISYETFK | |
| | KHILNLAKGKGRISKTKKEYLLEERDINRFSVQKDFINRNLV | |
| | DTRYATRGLMNLLRSYFRVNNLDVKVKSINGGFTSFLRRK | |
| | WKFKKERNKGYKHHAEDALIIANADFIFKEWKKLDKAKK | |
| | VMENQMFEEKQAESMPEIETEQEYKEIFITPHQIKHIKDFKD | |
| | YKYSHRVDKKPNRELINDTLYSTRKDDKGNTLIVNNLNGLY | |
| | DKDNDKLKKLINKSPEKLLMYHHDPQTYQKLKLIMEQYG | |
| | DEKNPLYKYYEETGNYLTKYSKKDNGPVIKKIKYYGNKLN | |
| | AHLDITDDYPNSRNKVVKLSLKPYRFDVYLDNGVYKFVTV | |
| | KNLDVIKKENYYEVNSKCYEEAKKLKKISNQAEFIASFYNN | |
| | DLIKINGELYRVIGVNNDLLNRIEVNMIDITYREYLENMNDK | |
| | RPPRIIKTIASKTQSIKKYSTDILGNLYEVKSKKHPQIIKKGSG | |
| | GSSGGSSGSETPGTSESATPESSGGSSGGSSTLNIEDEYRLHE | |
| | TSKEPDVSLGSTWLSDFPQAWAETGGMGLAVRQAPLIIPLK | |
| | ATSTPVSIKQYPMSQEARLGIKPHIQRLLDQGILVPCQSPWN | |
| | TPLLPVKKPGTNDYRPVQDLREVNKRVEDIHPTVPNPYNLL | |
| | SGLPPSHQWYTVLDLKDAFFCLRLHPTSQPLFAFEWRDPEM | |
| | GISGQLTWTRLPQGFKNSPTLFDEALHRDLADFRIQHPDLIL | |
| | LQYVDDLLLAATSELDCQQGTRALLQTLGNLGYRASAKKA | |
| | QICQKQVKYLGYLLKEGQRWLTEARKETVMGQPTPKTPRQ | |
| | LREFLGTAGFCRLWIPGFAEMAAPLYPLTKTGTLFNWGPDQ | |
| | QKAYQEIKQALLTAPALGLPDLTKPFELFVDEKQGYAKGVL | |
| | TQKLGPWRRPVAYLSKKLDPVAAGWPPCLRMVAAIAVLTK | |
| | DAGKLTMGQPLVILAPHAVEALVKQPPDRWLSNARMTHYQ | |
| | ALLLDTDRVQFGPVVALNPATLLPLPEEGLQHNCLDILAEAH | |
| | GTRPDLTDQPLPDADHTWYTDGSSLLQEGQRKAGAAVTTE | |
| | TEVIWAKALPAGTSAQRAELIALTQALKMAEGKKLNVYTD | |
| | SRYAFATAHIHGEIYRRRGLLTSEGKEIKNKDEILALLKALFL | |
| | PKRLSIIHCPGHQKGHSAEARGNRMADQAARKAAITETPDT | |
| | STLLIENSSPSGGSKRTADGSEFEPKKKRKV | |
| SPCAS9(H840A)- | MKRTADGSEFESPKKKRKVDKKYSIGLDIGTNSVGWAVITD | SEQ ID NO: |
| VRQR- | EYKVPSKKFKVLGNTDRHSIKKNLIGALLFDSGETAEATRL | 136 |
| MALONEY | KRTARRRYTRRKNRICYLQEIFSNEMAKVDDSFFHRLEESFL | |
| MURINE | VEEDKKHERHPIFGNIVDEVAYHEKYPTIYHLRKKLVDSTD | |
| LEUKEMIA | KADLRLIYLALAHMIKFRGHFLIEGDLNPDNSDVDKLFIQLV | |
| VIRUS | QTYNQLFEENPINASGVDAKAILSARLSKSRRLENLIAQLPG | |
| REVERSE | EKKNGLFGNLIALSLGLTPNFKSNFDLAEDAKLQLSKDTYD | |
| TRANSCRIPTASE | DDLDNLLAQIGDQYADLFLAAKNLSDAILLSDILRVNTEITK | |
| PRIME | APLSASMIKRYDEHHQDLTLLKALVRQQLPEKYKEIFFDQS | |
| EDITOR | KNGYAGYIDGGASQEEFYKFIKPILEKMDGTEELLVKLNRE | |
| | DLLRKQRTFDNGSIPHQIHLGELHAILRRQEDFYPFLKDNRE | |
| | KIEKILTFRIPYYVGPLARGNSRFAWMTRKSEETITPWNFEE | |
| | VVDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKIIKD | |
| | KDFLDNEENEDILEDIVLTLTLFEDREMIEERLKTYAHLFDD | |
| | KVMKQLKRRRYTGWGRLSRKLINGIRDKQSGKTILDFLKS | |
| | DGFANRNFMQLIHDDSLTFKEDIQKAQVSGQGDSLHEHIAN | |
| | LAGSPAIKKGILQTVKVVDELVKVMGRHKPENIVIEMAREN | |
| | QTTQKGQKNSRERMKRIEEGIKELGSQILKEHPVENTQLQN | |
| | EKLYLYYLQNGRDMYVDQELDINRLSDYDVDAIVPQSFLK | |
| | DDSIDNKVLTRSDKNRGKSDNVPSEEVVKKMKNYWRQLL | |
| | NAKLITQRKFDNLTKAERGGLSELDKAGFIKRQLVETRQITK | |
| | HVAQILDSRMNTKYDENDKLIREVKVITLKSKLVSDFRKDF | |
| | QFYKVREINNYHHAHDAYLNAVVGTALIKKYPKLESEFVY | |
| | GDYKVYDVRKMIAKSEQEIGKATAKYFFYSNIMNFFKTEIT | |
| | LANGEIRKRPLIETNGETGEIVWDKGRDFATVRKVLSMPQV | |
| | NIVKKTEVQTGGFSKESILPKRNSDKLIARKKDWDPKKYGG | |
| | FVSPTVAYSVLVVAKVEKGKSKKLKSVKELLGITIMERSSFE | |
| | KNPIDFLEAKGYKEVKKDLIIKLPKYSLFELENGRKRMLAS | |
| | ARELQKGNELALPSKYVNFLYLASHYEKLKGSPEDNEQKQ | |
| | LFVEQHKHYLDEIIEQISEFSKRVILADANLDKVLSAYNKHR | |
| | DKPIREQAENIIHLFTLTNLGAPAAFKYFDTTIDRKQYRSTKE | |
| | VLDATLIHQSITGLYETRIDLSQLGGDSGGSSGGSSGSETPGT | |
| | SESATPESSGGSSGGSSTLNIEDEYRLHETSKEPDVSLGSTW | |
| | LSDFPQAWAETGGMGLAVRQAPLIIPLKATSTPVSIKQYPMS | |
| | QEARLGIKPHIQRLLDQGILVPCQSPWNTPLLPVKKPGTND | |
| | YRPVQDLREVNKRVEDIHPTVPNPYNLLSGLPPSHQWYTVL | |
| | DLKDAFFCLRLHPTSQPLFAFEWRDPEMGISGQLTWTRLPQ | |
| | GFKNSPTLFNEALHRDLADFRIQHPDLILLQYVDDLLLAATS | |
| | ELDCQQGTRALLQTLGNLGYRASAKKAQICQKQVKYLGY | |
| | LLKEGQRWLTEARKETVMGQPTPKTPRQLREFLGKAGFCR | |
| | LFIPGFAEMAAPLYPLTKPGTLFNWGPDQQKAYQEIKQALL | |
| | TAPALGLPDLTKPFELFVDEKQGYAKGVLTQKLGPWRRPVA | |
| | YLSKKLDPVAAGWPPCLRMVAAIAVLTKDAGKLTMGQPLVI | |
| | LAPHAVEALVKQPPDRWLSNARMTHYQALLLDTDRVQFGP | |
| | VVALNPATLLPLPEEGLQHNCLDILAEAHGTRPDLTDQPLPD | |
| | ADHTWYTDGSSLLQEGQRKAGAAVTTETEVIWAKALPAGT | |
| | SAQRAELIALTQALKMAEGKKLNVYTDSRYAFATAHIHGEI | |
| | YRRRGWLTSEGKEIKNKDEILALLKALFLPKRLSIIHCPGHQ | |
| | KGHSAEARGNRMADQAARKAAITETPDTSTLLIENSSPSGG | |
| | SKRTADGSEFEPKKKRKV | |
| SPCAS9(H840A)- | MKRTADGSEFESPKKKRKVDKKYSIGLDIGTNSVGWAVITD | |
| VRER- | EYKVPSKKFKVLGNTDRHSIKKNLIGALLFDSGETAEATRL | |
| MALONEY | KRTARRRYTRRKNRICYLQEIFSNEMAKVDDSFFHRLEESFL | |
| MURINE | VEEDKKHERHPIFGNIVDEVAYHEKYPTIYHLRKKLVDSTD | |
| LEUKEMIA | KADLRLIYLALAHMIKFRGHFLIEGDLNPDNSDVDKLFIQLV | |
| VIRUS | QTYNQLFEENPINASGVDAKAILSARLSKSRRLENLIAQLPG | |
| REVERSE | EKKNGLFGNLIALSLGLTPNFKSNFDLAEDAKLQLSKDTYD | |
| TRANSCRIPTASE | DDLDNLLAQIGDQYADLFLAAKNLSDAILLSDILRVNTEITK | |
| PRIME | APLSASMIKRYDEHHQDLTLLKALVRQQLPEKYKEIFFDQS | |
| EDITOR | KNGYAGYIDGGASQEEFYKFIKPILEKMDGTEELLVKLNRE | |
| SEQ ID NO: | DLLRKQRTFDNGSIPHQIHLGELHAILRRQEDFYPFLKDNRE | |
| 137 | KIEKILTFRIPYYVGPLARGNSRFAWMTRKSEETITPWNFEE | |
| | VVDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKIIKD | |
| | KDFLDNEENEDILEDIVLTLTLFEDREMIEERLKTYAHLFDD | |
| | KVMKQLKRRRYTGWGRLSRKLINGIRDKQSGKTILDFLKS | |
| | DGFANRNFMQLIHDDSLTFKEDIQKAQVSGQGDSLHEHIAN | |
| | LAGSPAIKKGILQTVKVVDELVKVMGRHKPENIVIEMAREN | |
| | QTTQKGQKNSRERMKRIEEGIKELGSQILKEHPVENTQLQN | |
| | EKLYLYYLQNGRDMYVDQELDINRLSDYDVDAIVPQSFLK | |
| | DDSIDNKVLTRSDKNRGKSDNVPSEEVVKKMKNYWRQLL | |
| | NAKLITQRKFDNLTKAERGGLSELDKAGFIKRQLVETRQITK | |
| | HVAQILDSRMNTKYDENDKLIREVKVITLKSKLVSDFRKDF | |
| | QFYKVREINNYHHAHDAYLNAVVGTALIKKYPKLESEFVY | |
| | GDYKVYDVRKMIAKSEQEIGKATAKYFFYSNIMNFFKTEIT | |
| | LANGEIRKRPLIETNGETGEIVWDKGRDFATVRKVLSMPQV | |
| | NIVKKTEVQTGGFSKESILPKRNSDKLIARKKDWDPKKYGG | |
| | FVSPTVAYSVLVVAKVEKGKSKKLKSVKELLGITIMERSSFE | |
| | KNPIDFLEAKGYKEVKKDLIIKLPKYSLFELENGRKRMLAS | |
| | ARELQKGNELALPSKYVNFLYLASHYEKLKGSPEDNEQKQ | |
| | LFVEQHKHYLDEIIEQISEFSKRVILADANLDKVLSAYNKHR | |
| | DKPIREQAENIIHLFTLTNLGAPAAFKYFDTTIDRKEYRSTKE | |
| | VLDATLIHQSITGLYETRIDLSQLGGDSGGSSGGSSGSETPGT | |
| | SESATPESSGGSSGGSSTLNIEDEYRLHETSKEPDVSLGSTW | |
| | LSDFPQAWAETGGMGLAVRQAPLIIPLKATSTPVSIKQYPMS | |
| | QEARLGIKPHIQRLLDQGILVPCQSPWNTPLLPVKKPGTND | |
| | YRPVQDLREVNKRVEDIHPTVPNPYNLLSGLPPSHQWYTVL | |
| | DLKDAFFCLRLHPTSQPLFAFEWRDPEMGISGQLTWTRLPQ | |
| | GFKNSPTLFNEALHRDLADFRIQHPDLILLQYVDDLLLAATS | |
| | ELDCQQGTRALLQTLGNLGYRASAKKAQICQKQVKYLGY | |
| | LLKEGQRWLTEARKETVMGQPTPKTPRQLREFLGKAGFCR | |
| | LFIPGFAEMAAPLYPLTKPGTLFNWGPDQQKAYQEIKQALL | |
| | TAPALGLPDLTKPFELFVDEKQGYAKGVLTQKLGPWRRPVA | |
| | YLSKKLDPVAAGWPPCLRMVAAIAVLTKDAGKLTMGQPLVI | |
| | LAPHAVEALVKQPPDRWLSNARMTHYQALLLDTDRVQFGP | |
| | VVALNPATLLPLPEEGLQHNCLDILAEAHGTRPDLTDQPLPD | |
| | ADHTWYTDGSSLLQEGQRKAGAAVTTETEVIWAKALPAGT | |
| | SAQRAELIALTQALKMAEGKKLNVYTDSRYAFATAHIHGEI | |
| | YRRRGWLTSEGKEIKNKDEILALLKALFLPKRLSIIHCPGHQ | |
| | KGHSAEARGNRMADQAARKAAITETPDTSTLLIENSSPSGG | |
| | SKRTADGSEFEPKKKRKV | |

In yet other embodiments, the prime editor contemplated herein may include a Cas9 nickase (e.g., Cas9 (H840A)) fused to a truncated version of M-MLV reverse transcriptase. In this embodiment, the reverse transcriptase also contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e. adeno-associated virus and lentivirus delivery). This embodiment is referred to as Cas9(H840A)-MMLV-RT(trunc) or “PE2-short” or “PE2-trunc” and has the following amino acid sequence:

| | |
|---|---|
| CAS9(H840A)- | MKRTADGSEFESPKKKRKVDKKYSIGLDIGTNSVGWAVITDEY |
| MMLV- | KVPSKKFKVLGNTDRHSIKKNLIGALLFDSGETAEATRLKRTA |
| RT(TRUNC) OR | RRRYTRRKNRICYLQEIFSNEMAKVDDSFFHRLEESFLVEEDK |
| PE2-SHORT | KHERHPIFGNIVDEVAYHEKYPTIYHLRKKLVDSTDKADLRLI |
| | YLALAHMIKFRGHFLIEGDLNPDNSDVDKLFIQLVQTYNQLFE |
| | ENPINASGVDAKAILSARLSKSRRLENLIAQLPGEKKNGLFGN |
| | LIALSLGLTPNFKSNFDLAEDAKLQLSKDTYDDDLDNLLAQIG |
| | DQYADLFLAAKNLSDAILLSDILRVNTEITKAPLSASMIKRYDE |
| | HHQDLTLLKALVRQQLPEKYKEIFFDQSKNGYAGYIDGGASQ |
| | EEFYKFIKPILEKMDGTEELLVKLNREDLLRKQRTFDNGSIPH |
| | QIHLGELHAILRRQEDFYPFLKDNREKIEKILTFRIPYYVGPLA |
| | RGNSRFAWMTRKSEETITPWNFEEVVDKGASAQSFIERMTNF |
| | DKNLPNEKVLPKHSLLYEYFTVYNELTKVKYVTEGMRKPAFL |
| | SGEQKKAIVDLLFKTNRKVTVKQLKEDYFKKIECFDSVEISGV |
| | EDRFNASLGTYHDLLKIIKDKDFLDNEENEDILEDIVLTLTLFE |
| | DREMIEERLKTYAHLFDDKVMKQLKRRRYTGWGRLSRKLIN |
| | GIRDKQSGKTILDFLKSDGFANRNFMQLIHDDSLTFKEDIQKA |
| | QVSGQGDSLHEHIANLAGSPAIKKGILQTVKVVDELVKVMGR |
| | HKPENIVIEMARENQTTQKGQKNSRERMKRIEEGIKELGSQIL |
| | KEHPVENTQLQNEKLYLYYLQNGRDMYVDQELDINRLSDYD |
| | VDAIVPQSFLKDDSIDNKVLTRSDKNRGKSDNVPSEEVVKKM |
| | KNYWRQLLNAKLITQRKFDNLTKAERGGLSELDKAGFIKRQL |
| | VETRQITKHVAQILDSRMNTKYDENDKLIREVKVITLKSKLVS |
| | DFRKDFQFYKVREINNYHHAHDAYLNAVVGTALIKKYPKLES |
| | EFVYGDYKVYDVRKMIAKSEQEIGKATAKYFFYSNIMNFFKT |
| | EITLANGEIRKRPLIETNGETGEIVWDKGRDFATVRKVLSMPQ |
| | VNIVKKTEVQTGGFSKESILPKRNSDKLIARKKDWDPKKYGG |
| | FDSPTVAYSVLVVAKVEKGKSKKLKSVKELLGITIMERSSFEK |
| | NPIDFLEAKGYKEVKKDLIIKLPKYSLFELENGRKRMLASAGE |
| | LQKGNELALPSKYVNFLYLASHYEKLKGSPEDNEQKQLFVEQ |
| | HKHYLDEIIEQISEFSKRVILADANLDKVLSAYNKHRDKPIREQ |
| | AENIIHLFTLTNLGAPAAFKYFDTTIDRKRYTSTKEVLDATLIH |
| | QSITGLYETRIDLSQLGGDSGGSSGGSSGSETPGTSESATPESSG |
| | GSSGGSSTLNIEDEYRLHETSKEPDVSLGSTWLSDFPQAWAETGGMG |
| | LAVRQAPLIIPLKATSTPVSIKQYPMSQEARLGIKPHIQRLLDQGILVPC |
| | QSPWNTPLLPVKKPGTNDYRPVQDLREVNKRVEDIHPTVPNPYNLLS |
| | GLPPSHQWYTVLDLKDAFFCLRLHPTSQPLFAFEWRDPEMGISGQLT |
| | WTRLPQGFKNSPTLFNEALHRDLADFRIQHPDLILLQYVDDLLLAATS |
| | ELDCQQGTRALLQTLGNLGYRASAKKAQICQKQVKYLGYLLKEGOR |
| | WLTEARKETVMGQPTPKTPRQLREFLGKAGFCRLFIPGFAEMAAPLY |
| | PLTKPGTLFNWGPDQQKAYQEIKQALLTAPALGLPDLTKPFELFVDE |
| | KQGYAKGVLTQKLGPWRRPVAYLSKKLDPVAAGWPPCLRMVAAIAVL |
| | TKDAGKLTMGQPLVILAPHAVEALVKOPPDRWLSNARMTHYQALLL |
| | DTDRVQFGPVVALNPATLLPLPEEGLQHNCLDNSRLINSGGSKRTAD |
| | GSEFEPKKKRKV (SEQ ID NO: 35) |
| | KEY: |
| | NUCLEAR LOCALIZATION SEQUENCE (NLS) |
| | TOP:(SEQ ID NO: 29), |
| | BOTTOM: (SEQ ID NO: 30) |
| | CAS9(H840A) (SEQ ID NO: 31) |
| | 33-AMINO ACID LINKER 1 (SEQ ID NO: 11) |
| | M-MLV TRUNCATED REVERSE TRANSCRIPTASE (SEQ ID NO: 36) |

See FIG. 75, which provides a bar graph comparing the efficiency (i.e., “% of total sequencing reads with the specified edits or indels”) of PE2, PE2-trunc, PE3, and PE3-trunc over different target sites in various cell lines. The data shows that the prime editors comprising the truncated RT variants were about as efficient as the prime editors comprising the non-truncated RT proteins.

In various embodiments, the prime editor contemplated herein may also include any variants of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the above indicated prime editor fusion sequences.

In certain embodiments, linkers may be used to link any of the peptides or peptide domains or moieties of the invention (e.g., a napDNAbp linked or fused to a polymerase, such as a reverse transcriptase).

### [5] Linkers and Other Domains

The Prime editors may comprise various other domains besides the napDNAbp (e.g., Cas9 domain) and the polymerase domain (e.g., RT domain). For example, in the case where the napDNAbp is a Cas9 and the polymerase is a RT, the Prime editors may comprise one or more linkers that join the Cas9 domain with the RT domain. The linkers may also join other functional domains, such as nuclear localization sequences (NLS) or a FEN1 (or other flap endonuclease) to the Prime editors or a domain thereof.

In addition, in embodiments involving trans prime editing, linkers may be used to link tPERT recruitment protein to a prime editor, e.g., between the tPERt recruitment protein and the napDNAbp. See e.g., FIG. 3G for an exemplary schematic of a trans prime editor (tPE) that includes linkers to separately fuse a polymerase domain and a recruiting protein domain to a napDNAbp.

### A. Linkers

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a polymerase (e.g., a reverse transcriptase). In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In certain embodiments, the linkers are nucleotide linkers and can refer to those linkers that join a pegRNA to an additional nucleotide moiety, as described herein, such as, but not limited to, an aptamer (e.g., prequeosin₁-1 riboswitch aptamer or “evopreQ₁-1”) or a variant thereof, a pseudoknot (the MMLV viral genome pseudoknot or “Mpknot-1”) or a variant thereof, a tRNA (e.g., the modified tRNA used by MMLV as a primer for reverse transcription) or a variant thereof, or a G-quadruplex or a variant thereof. Exemplary nucleotide sequences of such linkers are provided throughout herein, and include, but are not limited to SEQ ID NOs: 225-236.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 138), (G)ₙ(SEQ ID NO: 139), (EAAAK)ₙ (SEQ ID NO: 12), (GGS)ₙ (SEQ ID NO: 140), (SGGS)ₙ(SEQ ID NO: 8), (XP)ₙ (SEQ ID NO: 141), or any combination thereof, wherein n is independently an integer between 1 and 30, and wherein X is any amino acid. In some embodiments, the linker comprises the amino acid sequence (GGS)N(SEQ ID NO: 140), wherein n is 1, 3, or 7. In some embodiments, the linker comprises the amino acid sequence SGSETPGTSESATPES (SEQ ID NO: 142). In some embodiments, the linker comprises the amino acid sequence SGGSSGGSSGSETPGTSESATPESSGGSSGGS (SEQ ID NO: 143). In some embodiments, the linker comprises the amino acid sequence SGGSGGSGGS (SEQ ID NO: 144). In some embodiments, the linker comprises the amino acid sequence SGGS (SEQ ID NO: 8). In other embodiments, the linker comprises the amino acid sequence

(SEQ ID NO: 131)
SGGSSGGSSGSETPGTSESATPESAGSYPYDVPDYAGSAAPAAKKKK
LDGSGSGGSSGGS.

In certain embodiments, linkers may be used to link any of the peptides or peptide domains or moieties of the invention (e.g., a napDNAbp linked or fused to a polymerase, such as a reverse transcriptase).

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 138), (G)ₙ (SEQ ID NO: 139), (EAAAK)ₙ (SEQ ID NO: 12), (GGS). (SEQ ID NO: 140), (SGGS)ₙ (SEQ ID NO: 8), (XP)ₙ (SEQ ID NO: 141), or any combination thereof, wherein n is independently an integer between 1 and 30, and wherein X is any amino acid. In some embodiments, the linker comprises the amino acid sequence (GGS)N(SEQ ID NO: 140), wherein n is 1, 3, or 7. In some embodiments, the linker comprises the amino acid sequence SGSETPGTSESATPES (SEQ ID NO: 142). In some embodiments, the linker comprises the amino acid sequence SGGSSGGSSGSETPGTSESATPESSGGSSGGS (SEQ ID NO: 143). In some embodiments, the linker comprises the amino acid sequence SGGSGGSGGS (SEQ ID NO: 144). In some embodiments, the linker comprises the amino acid sequence SGGS (SEQ ID NO: 8).

In particular, the following linkers can be used in various embodiments to join prime editor domains with one another:

(SEQ ID NO: 140)
GGS;
(SEQ ID NO: 145)
GGSGGS;
(SEQ ID NO: 146)
GGSGGSGGS;
(SEQ ID NO: 11)
SGGSSGGSSGSETPGTSESATPESSGGSSGGSS;
(SEQ ID NO: 142)
SGSETPGTSESATPES;
(SEQ ID NO: 131)
SGGSSGGSSGSETPGTSESATPESAGSYPYDVPDYAGSAAPAAKKKK
LDGSGSGGSSGGS.

### B. Nuclear Localization Sequence (NLS)

In various embodiments, the Prime editors may comprise one or more nuclear localization sequences (NLS), which help promote translocation of a protein into the cell nucleus. Such sequences are well-known in the art and can include the following examples:

| DESCRIPTION | SEQUENCE | SEQ ID NO: |
|---|---|---|
| NLS OF SV40 | PKKKRKV | SEQ ID NO: |
| LARGE T-AG | | 26 |
| NLS | MKRTADGSEFESPKKKRKV | SEQ ID NO: |
| | | 29 |
| NLS | MDSLLMNRRKFLYQFKNVRW | SEQ ID NO: |
| | AKGRRETYLC | 27 |
| NLS OF | AVKRPAATKKAGQAKKKKLD | SEQ ID NO: |
| NUCLEOPLASMIN | | 147 |
| NLS OF EGL-13 | MSRRRKANPTKLSENAKKLA | SEQ ID NO: |
| | KEVEN | 148 |
| NLS OF C-MYC | PAAKRVKLD | SEQ ID NO: |
| | | 149 |
| NLS OF TUS- | KLKIKRPVK | SEQ ID NO: |
| PROTEIN | | 150 |
| NLS OF | VSRKRPRP | SEQ ID NO: |
| POLYOMA | | 151 |
| LARGE T-AG | | |
| NLS OF | EGAPPAKRAR | SEQ ID NO: |
| HEPATITIS D | | 152 |
| VIRUS | | |
| ANTIGEN | | |
| NLS OF | PPQPKKKPLDGE | SEQ ID NO: |
| MURINE P53 | | 153 |
| NLS OF PE1 | SGGSKRTADGSEFEPKKKRK | SEQ ID NO: |
| AND PE2 | V | 30 |

The NLS examples above are non-limiting. The Prime editors may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the prime editors and constructs encoding the prime editors disclosed herein further comprise one or more, preferably, at least two nuclear localization signals. In certain embodiments, the prime editors comprise at least two NLSs. In embodiments with at least two NLSs, the NLSs can be the same NLSs or they can be different NLSs. In addition, the NLSs may be expressed as part of a fusion protein with the remaining portions of the prime editors. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 26), MDSLLMNRRKFLYQFKNVRWAKGRRETYLC (SEQ ID NO: 27), KRTADGSEFESPKKKRKV (SEQ ID NO: 154), or KRTADGSEFEPKKKRKV (SEQ ID NO: 155). In other embodiments, NLS comprises the amino acid sequences

(SEQ ID NO: 156)
NLSKRPAAIKKAGQAKKKK,
(SEQ ID NO: 149)
PAAKRVKLD,
(SEQ ID NO: 157)
RQRRNELKRSF,
(SEQ ID NO: 158)
NQSSNFGPMKGGNFGGRSSGPYGGGGQYFAKPRNQGGY.

In one aspect of the disclosure, a prime editor may be modified with one or more nuclear localization signals (NLS), preferably at least two NLSs. In certain embodiments, the prime editors are modified with two or more NLSs. The disclosure contemplates the use of any nuclear localization signal known in the art at the time of the disclosure, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 26)); (ii) a bipartite motif consisting of two basic domains separated by a variable number of spacer amino acids and exemplified by the Xenopus nucleoplasmin NLS (KRXXXXXXXXXXKKKL (SEQ ID NO: 159)); and (iii) noncanonical sequences such as M9 of the hnRNP A1 protein, the influenza virus nucleoprotein NLS, and the yeast Gal4 protein NLS (Dingwall and Laskey 1991).

Nuclear localization signals appear at various points in the amino acid sequences of proteins. NLS's have been identified at the N-terminus, the C-terminus and in the central region of proteins. Thus, the disclosure provides prime editors that may be modified with one or more NLSs at the C-terminus, the N-terminus, as well as at in internal region of the prime editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a prime editor to include one or more NLSs. In one aspect, the prime editors may be engineered to express a prime editor protein that is translationally fused at its N-terminus or its C-terminus (or both) to one or more NLSs, i.e., to form a prime editor-NLS fusion construct. In other embodiments, the prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a prime editor and one or more NLSs.

The prime editors described herein may also comprise nuclear localization signals which are linked to a prime editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the prime editor by any suitable strategy that effectuates forming a bond (e.g., covalent linkage, hydrogen bonding) between the prime editor and the one or more NLSs.

### C. Flap Endonucleases (e.g., FEN1)

In various embodiments, the Prime editors may comprise one or more flap endonucleases (e.g., FEN1), which refers to an enzyme that catalyzes the removal of 5′ single strand DNA flaps. These are enzymes that process the removal of 5′ flaps formed during cellular processes, including DNA replication. The prime editing methods herein described may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference). An exemplary flap endonuclease is FEN1, which can be represented by the amino acid sequence of SEQ ID NO: 15.

The flap endonucleases may also include any FEN1 variant, mutant, or other flap endonuclease ortholog, homolog, or variant. Non-limiting FEN1 variant examples are as follows:

| | | SEQ |
|---|---|---|
| Description | Sequence | ID NO: |
| FEN1 | MGIQGLAKLIADVAPSAIRENDIKSYFGRKVAIDASMSIY | SEQ ID NO: |
| K168R | QFLIAVRQGGDVLQNEEGETTSHLMGMFYRTIRMMENG | 160 |
| (relative | IKPVYVFDGKPPQLKSGELAKRSERRAEAEKQLQQAQA | |
| to FEN1 | AGAEQEVEKFTKRLVKVTKQHNDECKHLLSLMGIPYLD | |
| wt) | APSEAEASCAALVRAGKVYAAATEDMDCLTFGSPVLMR | |
| | HLTASEAKKLPIQEFHLSRILQELGLNQEQFVDLCILLGS | |
| | DYCESIRGIGPKRAVDLIQKHKSIEEIVRRLDPNKYPVPEN | |
| | WLHKEAHQLFLEPEVLDPESVELKWSEPNEEELIKFMCG | |
| | EKQFSEERIRSGVKRLSKSRQGSTQGRLDDFFKVTGSLSS | |
| | AKRKEPEPKGSTKKKAKTGAAGKFKRGK | |
| FEN1 | MGIQGLAKLIADVAPSAIRENDIKSYFGRKVAIDASMSIY | SEQ ID NO: |
| S187A | QFLIAVRQGGDVLQNEEGETTSHLMGMFYRTIRMMENG | 161 |
| (relative | IKPVYVFDGKPPQLKSGELAKRSERRAEAEKQLQQAQA | |
| to FEN1 | AGAEQEVEKFTKRLVKVTKQHNDECKHLLSLMGIPYLD | |
| wt) | APSEAEASCAALVKAGKVYAAATEDMDCLTFGAPVLM | |
| | RHLTASEAKKLPIQEFHLSRILQELGLNQEQFVDLCILLGS | |
| | DYCESIRGIGPKRAVDLIQKHKSIEEIVRRLDPNKYPVPEN | |
| | WLHKEAHQLFLEPEVLDPESVELKWSEPNEEELIKFMCG | |
| | EKQFSEERIRSGVKRLSKSRQGSTQGRLDDFFKVTGSLSS | |
| | AKRKEPEPKGSTKKKAKTGAAGKFKRGK | |
| FEN1 | MGIQGLAKLIADVAPSAIRENDIKSYFGRKVAIDASMSIY | SEQ ID NO: |
| K354R | QFLIAVRQGGDVLQNEEGETTSHLMGMFYRTIRMMENG | 162 |
| (relative | IKPVYVFDGKPPQLKSGELAKRSERRAEAEKQLQQAQA | |
| to FEN1 | AGAEQEVEKFTKRLVKVTKQHNDECKHLLSLMGIPYLD | |
| wt) | APSEAEASCAALVKAGKVYAAATEDMDCLTFGSPVLMR | |
| | HLTASEAKKLPIQEFHLSRILQELGLNQEQFVDLCILLGS | |
| | DYCESIRGIGPKRAVDLIQKHKSIEEIVRRLDPNKYPVPEN | |
| | WLHKEAHQLFLEPEVLDPESVELKWSEPNEEELIKFMCG | |
| | EKQFSEERIRSGVKRLSKSRQGSTQGRLDDFFKVTGSLSS | |
| | ARRKEPEPKGSTKKKAKTGAAGKFKRGK | |
| GEN1 | MGVNDLWQILEPVKQHIPLRNLGGKTIAVDLSLWVCEA | SEQ ID NO: |
| | QTVKKMMGSVMKPHLRNLFFRISYLTQMDVKLVFVME | 163 |
| | GEPPKLKADVISKRNQSRYGSSGKSWSQKTGRSHFKSVL | |
| | RECLHMLECLGIPWVQAAGEAEAMCAYLNAGGHVDGC | |
| | LTNDGDTFLYGAQTVYRNFTMNTKDPHVDCYTMSSIKS | |
| | KLGLDRDALVGLAILLGCDYLPKGVPGVGKEQALKLIQI | |
| | LKGQSLLQRFNRWNETSCNSSPQLLVTKKLAHCSVCSHP | |
| | GSPKDHERNGCRLCKSDKYCEPHDYEYCCPCEWHRTEH | |
| | DRQLSEVENNIKKKACCCEGFPFHEVIQEFLLNKDKLVK | |
| | VIRYQRPDLLLFQRFTLEKMEWPNHYACEKLLVLLTHY | |
| | DMIERKLGSRNSNQLQPIRIVKTRIRNGVHCFEIEWEKPE | |
| | HYAMEDKQHGEFALLTIEEESLFEAAYPEIVAVYQKQKL | |
| | EIKGKKQKRIKPKENNLPEPDEVMSFQSHMTLKPTCEIFH | |
| | KQNSKLNSGISPDPTLPQESISASLNSLLLPKNTPCLNAQE | |
| | QFMSSLRPLAIQQIKAVSKSLISESSQPNTSSHNISVIADLH | |
| | LSTIDWEGTSFSNSPAIQRNTFSHDLKSEVESELSAIPDGF | |
| | ENIPEQLSCESERYTANIKKVLDEDSDGISPEEHLLSGITD | |
| | LCLQDLPLKERIFTKLSYPQDNLQPDVNLKTLSILSVKES | |
| | CIANSGSDCTSHLSKDLPGIPLQNESRDSKILKGDQLLQE | |
| | DYKVNTSVPYSVSNTVVKTCNVRPPNTALDHSRKVDM | |
| | QTTRKILMKKSVCLDRHSSDEQSAPVFGKAKYTTQRMK | |
| | HSSQKHNSSHFKESGHNKLSSPKIHIKETEQCVRSYETAE | |
| | NEESCFPDSTKSSLSSLQCHKKENNSGTCLDSPLPLRQRL | |
| | KLRFQST | |
| ERCC5 | MGVQGLWKLLECSGRQVSPEALEGKILAVDISIWLNQAL | SEQ ID NO: |
| | KGVRDRHGNSIENPHLLTLFHRLCKLLFFRIRPIFVFDGD | 164 |
| | APLLKKQTLVKRRQRKDLASSDSRKTTEKLLKTFLKRQA | |
| | IKTAFRSKRDEALPSLTQVRRENDLYVLPPLQEEEKHSSE | |
| | EEDEKEWQERMNQKQALQEEFFHNPQAIDIESEDFSSLPP | |
| | EVKHEILTDMKEFTKRRRTLFEAMPEESDDFSQYQLKGL | |
| | LKKNYLNQHIEHVQKEMNQQHSGHIRRQYEDEGGFLKE | |
| | VESRRVVSEDTSHYILIKGIQAKTVAEVDSESLPSSSKMH | |
| | GMSFDVKSSPCEKLKTEKEPDATPPSPRTLLAMQAALLG | |
| | SSSEEELESENRRQARGRNAPAAVDEGSISPRTLSAIKRA | |
| | LDDDEDVKVCAGDDVQTGGPGAEEMRINSSTENSDEGL | |
| | KVRDGKGIPFTATLASSSVNSAEEHVASTNEGREPTDSV | |
| | PKEQMSLVHVGTEAFPISDESMIKDRKDRLPLESAVVRH | |
| | SDAPGLPNGRELTPASPTCTNSVSKNETHAEVLEQQNEL | |
| | CPYESKFDSSLLSSDDETKCKPNSASEVIGPVSLQETSSIV | |
| | SVPSEAVDNVENVVSFNAKEHENFLETIQEQQTTESAGQ | |
| | DLISIPKAVEPMEIDSEESESDGSFIEVQSVISDEELQAEFP | |
| | ETSKPPSEQGEEELVGTREGEAPAESESLLRDNSERDDVD | |
| | GEPQEAEKDAEDSLHEWQDINLEELETLESNLLAQQNSL | |
| | KAQKQQQERIAATVTGQMFLESQELLRLFGIPYIQAPME | |
| | AEAQCAILDLTDQTSGTITDDSDIWLFGARHVYRNFFNK | |
| | NKFVEYYQYVDFHNQLGLDRNKLINLAYLLGSDYTEGIP | |
| | TVGCVTAMEILNEFPGHGLEPLLKFSEWWHEAQKNPKIR | |
| | PNPHDTKVKKKLRTLQLTPGFPNPAVAEAYLKPVVDDS | |
| | KGSFLWGKPDLDKIREFCQRYFGWNRTKTDESLFPVLK | |
| | QLDAQQTQLRIDSFFRLAQQEKEDAKRIKSQRLNRAVTC | |
| | MLRKEKEAAASEIEAVSVAMEKEFELLDKAKRKTQKRG | |
| | ITNTLEESSSLKRKRLSDSKRKNTCGGFLGETCLSESSDG | |
| | SSSEDAESSSLMNVQRRTAAKEPKTSASDSQNSVKEAPV | |
| | KNGGATTSSSSDSDDDGGKEKMVLVTARSVFGKKRRKL | |
| | RRARGRKRKT | |

In various embodiments, the prime editor contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences.

Other endonucleases that may be utilized by the instant methods to facilitate removal of the 5′ end single strand DNA flap include, but are not limited to (1) trex 2, (2) exo1 endonuclease (e.g., Keijzers et al., Biosci Rep. 2015, 35(3): e00206)

### Trex 2

3′ three prime repair exonuclease 2 (TREX2)—human

Accession No. NM_080701

(SEQ ID NO: 165)
MSEAPRAETFVFLDLEATGLPSVEPEIAELSLFAVHRSSLENPEHDESGA
LVLPRVLDKLTLCMCPERPFTAKASEITGLSSEGLARCRKAGFDGAVVRT
LQAFLSRQAGPICLVAHNGFDYDFPLLCAELRRLGARLPRDTVCLDTLPA
LRGLDRAHSHGTRARGRQGYSLGSLFHRYFRAEPSAAHSAEGDVHTLLLI
FLHRAAELLAWADEQARGWAHIEPMYLPPDDPSLEA.

3′ three prime repair exonuclease 2 (TREX2)—mouse

Accession No. NM_011907

(SEQ ID NO: 166)
MSEPPRAETFVFLDLEATGLPNMDPEIAEISLFAVHRSSLENPERDDSGS
LVLPRVLDKLTLCMCPERPFTAKASEITGLSSESLMHCGKAGFNGAVVRT
LQGFLSRQEGPICLVAHNGFDYDFPLLCTELQRLGAHLPQDTVCLDTLPA
LRGLDRAHSHGTRAQGRKSYSLASLFHRYFQAEPSAAHSAEGDVHTLLLI
FLHRAPELLAWADEQARSWAHIEPMYVPPDGPSLEA.

3′ three prime repair exonuclease 2 (TREX2)—rat

Accession No. NM_001107580

(SEQ ID NO: 167)
MSEPLRAETFVFLDLEATGLPNMDPEIAEISLFAVHRSSLENPERDDSGS
LVLPRVLDKLTLCMCPERPFTAKASEITGLSSEGLMNCRKAAFNDAVVRT
LQGFLSRQEGPICLVAHNGFDYDFPLLCTELQRLGAHLPRDTVCLDTLPA
LRGLDRVHSHGTRAQGRKSYSLASLFHRYFQAEPSAAHSAEGDVNTLLLI
FLHRAPELLAWADEQARSWAHIEPMYVPPDGPSLEA.

### ExoI

Human exonuclease 1 (EXO1) has been implicated in many different DNA metabolic processes, including DNA mismatch repair (MMR), micro-mediated end-joining, homologous recombination (HR), and replication. Human EXO1 belongs to a family of eukaryotic nucleases, Rad2/XPG, which also include FEN1 and GEN1. The Rad2/XPG family is conserved in the nuclease domain through species from phage to human. The EXO1 gene product exhibits both 5′ exonuclease and 5′ flap activity. Additionally, EXO1 contains an intrinsic 5′ RNase H activity. Human EXO1 has a high affinity for processing double stranded DNA (dsDNA), nicks, gaps, pseudo Y structures and can resolve Holliday junctions using its inherit flap activity. Human EXO1 is implicated in MMR and contain conserved binding domains interacting directly with MLH1 and MSH2. EXO1 nucleolytic activity is positively stimulated by PCNA, MutSα (MSH2/MSH6 complex), 14-3-3, MRN and 9-1-1 complex.

exonuclease 1 (EXO1) Accession No. NM_003686 (Homo sapiens exonuclease 1 (EXO1), transcript variant 3)—isoform A

(SEQ ID NO: 168)
MGIQGLLQFIKEASEPIHVRKYKGQVVAVDTYCWLHKGAIACAEKLAKGE
PTDRYVGFCMKFVNMLLSHGIKPILVFDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSAELSEDDLLSQYSLSFTKKTKKNSSEGN
KSLSFSEVFVPDLVNGPTNKKSVSTPPRTRNKFATFLQRKNEESGAVVVP
GTRSRFFCSSDSTDCVSNKVSIQPLDETAVTDKENNLHESEYGDQEGKRL
VDTDVARNSSDDIPNNHIPGDHIPDKATVFTDEESYSFESSKFTRTISPP
TLGTLRSCFSWSGGLGDFSRTPSPSPSTALQQFRRKSDSPTSLPENNMSD
VSQLKSEESSDDESHPLREEACSSQSQESGEFSLQSSNASKLSQCSSKDS
DSEESDCNIKLLDSQSDQTSKLRLSHFSKKDTPLRNKVPGLYKSSSADSL
STTKIKPLGPARASGLSKKPASIQKRKHHNAENKPGLQIKLNELWKNFGF
KKF.

exonuclease 1 (EXO1) Accession No. NM_006027 (Homo sapiens exonuclease 1 (EXO1), transcript variant 3)—isoform B

(SEQ ID NO: 169)
MGIQGLLQFIKEASEPIHVRKYKGQVVAVDTYCWLHKGAIACAEKLAKGE
PTDRYVGFCMKFVNMLLSHGIKPILVFDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSAELSEDDLLSQYSLSFTKKTKKNSSEGN
KSLSFSEVFVPDLVNGPTNKKSVSTPPRTRNKFATFLQRKNEESGAVVVP
GTRSRFFCSSDSTDCVSNKVSIQPLDETAVTDKENNLHESEYGDQEGKRL
VDTDVARNSSDDIPNNHIPGDHIPDKATVFTDEESYSFESSKFTRTISPP
TLGTLRSCFSWSGGLGDFSRTPSPSPSTALQQFRRKSDSPTSLPENNMSD
VSQLKSEESSDDESHPLREEACSSQSQESGEFSLQSSNASKLSQCSSKDS
DSEESDCNIKLLDSQSDQTSKLRLSHFSKKDTPLRNKVPGLYKSSSADSL
STTKIKPLGPARASGLSKKPASIQKRKHHNAENKPGLQIKLNELWKNFGF
KKDSEKLPPCKKPLSPVRDNIQLTPEAEEDIFNKPECGRVQRAIFQ.

exonuclease 1 (EXO1) Accession No. NM_001319224 (Homo sapiens exonuclease 1 (EXO1), transcript variant 4)—isoform C

(SEQ ID NO: 170)
MGIQGLLQFIKEASEPIHVRKYKGQVVAVDTYCWLHKGAIACAEKLAKGE
PTDRYVGFCMKFVNMLLSHGIKPILVFDGCTLPSKKEVERSRRERRQANL
LKGKQLLREGKVSEARECFTRSINITHAMAHKVIKAARSQGVDCLVAPYE
ADAQLAYLNKAGIVQAIITEDSDLLAFGCKKVILKMDQFGNGLEIDQARL
GMCRQLGDVFTEEKFRYMCILSGCDYLSSLRGIGLAKACKVLRLANNPDI
VKVIKKIGHYLKMNITVPEDYINGFIRANNTFLYQLVFDPIKRKLIPLNA
YEDDVDPETLSYAGQYVDDSIALQIALGNKDINTFEQIDDYNPDTAMPAH
SRSHSWDDKTCQKSANVSSIWHRNYSPRPESGTVSDAPQLKENPSTVGVE
RVISTKGLNLPRKSSIVKRPRSELSEDDLLSQYSLSFTKKTKKNSSEGNK
SLSFSEVFVPDLVNGPTNKKSVSTPPRTRNKFATFLQRKNEESGAVVVPG
TRSRFFCSSDSTDCVSNKVSIQPLDETAVTDKENNLHESEYGDQEGKRLV
DTDVARNSSDDIPNNHIPGDHIPDKATVFTDEESYSFESSKFTRTISPPT
LGTLRSCFSWSGGLGDFSRTPSPSPSTALQQFRRKSDSPTSLPENNMSDV
SQLKSEESSDDESHPLREEACSSQSQESGEFSLQSSNASKLSQCSSKDSD
SEESDCNIKLLDSQSDQTSKLRLSHFSKKDTPLRNKVPGLYKSSSADSLS
TTKIKPLGPARASGLSKKPASIQKRKHHNAENKPGLQIKLNELWKNFGFK
KDSEKLPPCKKPLSPVRDNIQLTPEAEEDIFNKPECGRVQRAIFQ.

### D. Inteins and Split-Inteins

It will be understood that in some embodiments (e.g., delivery of a prime editor in vivo using AAV particles), it may be advantageous to split a polypeptide (e.g., a deaminase or a napDNAbp) or a fusion protein (e.g., a prime editor) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the −12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

Exemplary sequences are represented by SEQ ID NOs: 16-23.

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostoc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product, e.g., as shown in FIGS. 66 and 67 with regard to the formation of a complete Prime editor from two separately-expressed halves.

### E. RNA-Protein Interaction Domain

In various embodiments, two separate protein domains (e.g., a Cas9 domain and a polymerase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a prime editor, as well as to recruitment additional functionalities to a prime editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin is represented by SEQ ID NO: 24.

The amino acid sequence of the MCP or MS2cp is represented by SEQ ID NO: 25.

### F. UGI Domain

In other embodiments, the prime editors described herein may comprise one or more uracil glycosylase inhibitor domains. The term “uracil glycosylase inhibitor (UGI)” or “UGI domain,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 171. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 171. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 171. In some embodiments, a UGI comprises an amino acid sequence homologous to the amino acid sequence set forth in SEQ ID NO: 171, or an amino acid sequence homologous to a fragment of the amino acid sequence set forth in SEQ ID NO: 171. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 171. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 171. In some embodiments, the UGI comprises the following amino acid sequence:

Uracil-DNA glycosylase inhibitor:

(SEQ ID NO: 171)
>sp|P14739|UNGI_BPPB2
MTNLSDIIEKETGKQLVIQESILMLPEEVEEVIGNKPESDILVHTAYDES
TDENVMLLTSDAPEYKPWALVIQDSNGENKIKML.

The prime editors described herein may comprise more than one UGI domain, which may be separated by one or more linkers as described herein.

### G. Additional PE Elements

In certain embodiments, the prime editors described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE 1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, MBD4 inhibitor or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 175 (human TDG).

Some exemplary glycosylases are provided below. The catalytically inactivated variants of any of these glycosylase domains are iBERs that may be fused to the napDNAbp or polymerase domain of the prime editors provided in this disclosure.

OGG (human)
(SEQ ID NO: 172)
MPARALLPRRMGHRTLASTPALWASIPCPRSELRLDLVLPSGQSFRWREQ
SPAHWSGVLADQVWTLTQTEEQLHCTVYRGDKSQASRPTPDELEAVRKYF
QLDVTLAQLYHHWGSVDSHFQEVAQKFQGVRLLRQDPIECLFSFICSSNN
NIARITGMVERLCQAFGPRLIQLDDVTYHGFPSLQALAGPEVEAHLRKLG
LGYRARYVSASARAILEEQGGLAWLQQLRESSYEEAHKALCILPGVGTKV
ADCICLMALDKPQAVPVDVHMWHIAQRDYSWHPTTSQAKGPSPQTNKELG
NFFRSLWGPYAGWAQAVLFSADLRQSRHAQEPPAKRRKGSKGPEG
MPG (human)
(SEQ ID NO: 173)
MVTPALQMKKPKQFCRRMGQKKQRPARAGQPHSSSDAAQAPAEQPHSSSD
AAQAPCPRERCLGPPTTPGPYRSIYFSSPKGHLTRLGLEFFDQPAVPLAR
AFLGQVLVRRLPNGTELRGRIVETEAYLGPEDEAAHSRGGRQTPRNRGMF
MKPGTLYVYIIYGMYFCMNISSQGDGACVLLRALEPLEGLETMRQLRSTL
RKGTASRVLKDRELCSGPSKLCQALAINKSFDQRDLAQDEAVWLERGPLE
PSEPAVVAAARVGVGHAGEWARKPLRFYVRGSPWVSVVDRVAEQDTQA
MBD4 (human)
(SEQ ID NO: 174)
MGTTGLESLSLGDRGAAPTVTSSERLVPDPPNDLRKEDVAMELERVGEDE
EQMMIKRSSECNPLLQEPIASAQFGATAGTECRKSVPCGWERVVKQRLFG
KTAGRFDVYFISPQGLKFRSKSSLANYLHKNGETSLKPEDFDFTVLSKRG
IKSRYKDCSMAALTSHLQNQSNNSNWNLRTRSKCKKDVFMPPSSSSELQE
SRGLSNFTSTHLLLKEDEGVDDVNFRKVRKPKGKVTILKGIPIKKTKKGC
RKSCSGFVQSDSKRESVCNKADAESEPVAQKSQLDRTVCISDAGACGETL
SVTSEENSLVKKKERSLSSGSNFCSEQKTSGIINKFCSAKDSEHNEKYED
TFLESEEIGTKVEVVERKEHLHTDILKRGSEMDNNCSPTRKDFTGEKIFQ
EDTIPRTQIERRKTSLYFSSKYNKEALSPPRRKAFKKWTPPRSPFNLVQE
TLFHDPWKLLIATIFLNRTSGKMAIPVLWKFLEKYPSAEVARTADWRDVS
ELLKPLGLYDLRAKTIVKFSDEYLTKQWKYPIELHGIGKYGNDSYRIFCV
NEWKQVHPEDHKLNKYHDWLWENHEKLSLS
TDG (human)
(SEQ ID NO: 175)
MEAENAGSYSLQQAQAFYTFPFQQLMAEAPNMAVVNEQQMPEEVPAPAPA
QEPVQEAPKGRKRKPRTTEPKQPVEPKKPVESKKSGKSAKSKEKQEKITD
TFKVKRKVDRFNGVSEAELLTKTLPDILTFNLDIVIIGINPGLMAAYKGH
HYPGPGNHFWKCLFMSGLSEVQLNHMDDHTLPGKYGIGFTNMVERTTPGS
KDLSSKEFREGGRILVQKLQKYQPRIAVFNGKCIYEIFSKEVFGVKVKNL
EFGLQPHKIPDTETLCYVMPSSSARCAQFPRAQDKVHYYIKLKDLRDQLK
GIERNMDVQEVQYTFDLQLAQEDAKKMAVKEEKYDPGYEAAYGGAYGENP
CSSEPCGFSSNGLIESVELRGESAFSGIPNGQWMTQSFTDQIPSFSNHCG
TQEQEEESHA

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags, and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A prime editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011 and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments of the present disclosure, the activity of the prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the pegRNA may deliver the guide prior to the vector encoding the PE system. In some embodiments, the vectors encoding the PE system and pegRNA are delivered simultaneously. In certain embodiments, the simultaneously delivered vectors temporally deliver, e.g., the PE, pegRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or pegRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the pegRNA may be self-destroyed via cleavage of a target sequence present on the vector by the PE system. The cleavage may prevent continued transcription of a PE or a pegRNA from the vector. Although transcription may occur on the linearized vector for some amount of time, the expressed transcripts or proteins subject to intracellular degradation will have less time to produce off-target effects without continued supply from expression of the encoding vectors.

[6] Modified pegRNAs

The prime editing system described herein contemplates the use of any suitable pegRNAs, and in particular, pegRNAs which are modified to include one or more of the herein disclosed structural motifs which impart improved characteristics, such as increased stability and/or increased affinity for Cas9. The present inventors have surprisingly found that by appending certain nucleotide structural motifs to a pegRNA, e.g., to the terminus of the extension arm of a pegRNA, including but limited to, a prequeosin₁-1 riboswitch aptamer (“evopreQ₁-1”), a pseudoknot from the MMLV viral genome (“evopreQ₁-1”), and a modified tRNA used by MMLV RT as a primer for reverse transcription, a consistent increase in editing activity was achieved.

Canonical pegRNA Architecture

FIG. 3A shows one embodiment of a canonical pegRNA that may be modified and then use in the prime editing system disclosed herein. The canonical pegRNA (i.e., a pegRNA not including any of the modifications described here) comprises a traditional guide RNA (the green portion), which includes a ˜20 nt spacer sequence and a gRNA core region, and which binds with a napDNAbp. A canonical pegRNA also includes an extended RNA segment at the 5′ end, i.e., a 5′ extension, or at the 3′ end, i.e., a 3′ extension. The 5′extension includes a reverse transcription template sequence, a reverse transcription primer binding site, and an optional 5-20 nucleotide linker sequence. As shown in FIGS. 1A-1B, the RT primer binding site hybridizes to the free 3′ end that is formed after a nick is formed in the non-target strand of the R-loop, thereby priming reverse transcriptase for DNA polymerization in the 5′-3′ direction.

FIG. 3B shows another embodiment of a pegRNA usable in the prime editing system disclosed herein whereby a traditional guide RNA (the green portion) includes a ˜20 nt protospacer sequence and a gRNA core, which binds with the napDNAbp. In this embodiment, the guide RNA includes an extended RNA segment at the 3′ end, i.e., a 3′ extension. In this embodiment, the 3′extension includes a reverse transcription template sequence, and a reverse transcription primer binding site. As shown in FIGS. 1C-1D, the RT primer binding site hybridizes to the free 3′ end that is formed after a nick is formed in the non-target strand of the R-loop, thereby priming reverse transcriptase for DNA polymerization in the 5′-3′ direction.

FIG. 3C shows another embodiment of an pegRNA usable in the prime editing system disclosed herein whereby a traditional guide RNA (the green portion) includes a ˜20 nt protospacer sequence and a gRNA core, which binds with the napDNAbp. In this embodiment, the guide RNA includes an extended RNA segment at an intermolecular position within the gRNA core, i.e., an intramolecular extension. In this embodiment, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

Any of these canonical pegRNAs can be further modified to include one or more of the modifications described herein to increase the efficiency of prime editing.

In one embodiment, the position of the intermolecular RNA extension is not in the protospacer sequence of the guide RNA. In another embodiment, the position of the intermolecular RNA extension in the gRNA core. In still another embodiment, the position of the intermolecular RNA extension is any with the guide RNA molecule except within the protospacer sequence, or at a position which disrupts the protospacer sequence.

In one embodiment, the intermolecular RNA extension is inserted downstream from the 3′ end of the protospacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides downstream of the 3′ end of the protospacer sequence.

In other embodiments, the intermolecular RNA extension is inserted into the gRNA, which refers to the portion of the guide RNA corresponding or comprising the tracrRNA, which binds and/or interacts with the Cas9 protein or equivalent thereof (i.e, a different napDNAbp). Preferably the insertion of the intermolecular RNA extension does not disrupt or minimally disrupts the interaction between the tracrRNA portion and the napDNAbp.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, wherein the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The least one nucleotide change may include one or more single-base nucleotide changes, one or more deletions, and one or more insertions.

As depicted in FIG. 1G, the synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species (e.g., see FIG. 1E). This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand which becomes displaced as the 5′ flap species and which overlaps with the site to be edited.

In various embodiments of the pegRNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the pegRNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the pegRNAs are modified versions of a guide RNA. Guide RNAs may be expressed from an encoding nucleic acid, or synthesized chemically. Methods are well known in the art for obtaining or otherwise synthesizing guide RNAs and for determining the appropriate sequence of the guide RNA, including the protospacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in prime editing systems described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor (PE) to a target sequence may be assessed by any suitable assay. For example, the components of a prime editor (PE), including the guide sequence to be tested, may be provided to a host cell having the corresponding target sequence, such as by transfection with vectors encoding the components of a prime editor (PE) disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor (PE), including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 176) where NNNNNNNNNNNNXGG (SEQ ID NO: 177) (N is A, G, T, or C; and X can be anything). A unique target sequence in a genome may include an S. pyogenes Cas9 target site of the form MMMMMMMMMNNNNNNNNNNNXGG (SEQ ID NO: 178) where NNNNNNNNNNNXGG (SEQ ID NO: 179) (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW (SEQ ID NO: 180) where NNNNNNNNNNNNXXAGAAW (SEQ ID NO: 181) (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW (SEQ ID NO: 182) where NNNNNNNNNNNXXAGAAW (SEQ ID NO: 183) (N is A, G, T, or C; X can be anything; and W is A or T). For the S. pyogenes Cas9, a unique target sequence in a genome may include a Cas9 target site of the form MMMMMMMMNNNNNNNNNNNNXGGXG (SEQ ID NO: 184) where NNNNNNNNNNNNXGGXG (SEQ ID NO: 185) (N is A, G, T, or C; and X can be anything). A unique target sequence in a genome may include an S. pyogenes Cas9 target site of the form MMMMMMMMMNNNNNNNNNNNXGGXG (SEQ ID NO: 186) where NNNNNNNNNNNXGGXG (SEQ ID NO: 187) (N is A, G, T, or C; and X can be anything). In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 188)
NNNNNNNNGTTTTTGTACTCTCAAGATTTAGAAATAAATCTTGCAGAAGC
TACAAAGATAAGGCTTCATGCCGAAATCAACACCCTGTCATTTTATGGCA
GGGTGTTTTCGTTATTTAATTTTTT;
(2)
(SEQ ID NO: 189)
NNNNNNNNNNNNNNNNNNGTTTTTGTACTCTCAGAAATGCAGAAGCTACA
AAGATAAGGCTTCATGCCGAAATCAACACCCTGTCATTTTATGGCAGGGT
GTTTTCGTTATTTAATTTTTT;
(3)
(SEQ ID NO: 190)
NNNNNNNNNNNNNNNNNNNNGTTTTTGTACTCTCAGAAATGCAGAAGCTA
CAAAGATAAGGCTTCATGCCGAAATCAACACCCTGTCATTTTATGGCAGG
GTGTTTTTT;
(4)
(SEQ ID NO: 191)
NNNNNNNNNNNNNNNNNNNNGTTTTAGAGCTAGAAATAGCAAGTTAAAAT
AAGGCTAGTCCGTTATCAACTTGAAAAAGTGGCACCGAGTCGGTGCTTTT
TT;
(5)
(SEQ ID NO: 192)
NNNNNNNNNNNNNNNNNNNNGTTTTAGAGCTAGAAATAGCAAGTTAAAAT
AAGGCTAGTCCGTTATCAACTTGAAAAAGTGTTTTTTT;
AND
(6)
(SEQ ID NO: 193)
NNNNNNNNNNNNNNNNNNNNGTTTTAGAGCTAGAAATAGCAAGTTAAAAT
AAGGCTAGTCCGTTATCATTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-GUUUUAGAGCUAGAAAUAGCAAGUUAAAAUAAAGGCUAGUCCGUUAUCAACU UGAAAAAGUGGCACCGAGUCGGUGCUUUUU-3′ (SEQ ID NO: 194), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editor (PE) described herein.

In other embodiments, the pegRNAs include those depicted in FIG. 3D.

In still other embodiments, the pegRNAs may include those depicted in FIG. 3E.

FIG. 3D provides the structure of an embodiment of a pegRNA contemplated herein and which may be designed in accordance with the methodology defined in Example 2. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: an optional homology arm, a DNA synthesis template, and a primer binding site (PBS). In addition, the pegRNA may comprise an optional 3′ end modifier region (e1) and an optional 5′ end modifier region (e2). Still further, the pegRNA may comprise a transcriptional termination signal at the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

FIG. 3E provides the structure of another embodiment of a pegRNA contemplated herein and which may be designed in accordance with the methodology defined in Example 2. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: an optional homology arm, a DNA synthesis template, and a primer binding site (PBS). In addition, the pegRNA may comprise an optional 3′ end modifier region (e1) and an optional 5′ end modifier region (e2). Still further, the pegRNA may comprise a transcriptional termination signal on the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

In some embodiments, the PEgRNA or nicking guide RNA described herein comprises a chemically modified nucleobase or nucleobase analog. In some embodiments, the PEgRNA or nicking guide RNA comprises a modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages). In some embodiments, the PEgRNA comprises a 2′-O-methyl modification. In some embodiments, the PEgRNA comprises a phosphorothioate linkages between the first and last three nucleotides of the RNA.

In some embodiments, the PEgRNA or nicking guide RNA described herein comprises a chemical modification comprising a nebularine or a deoxynebularine. In some embodiments, the PEgRNA or nicking guide RNA comprises a chemical modification comprising a phosphorothioate linkage. In some embodiments, the PEgRNA or nicking guide RNA comprises a phosphorothioate linkage at a 5′ end or at a 3′ end. In some embodiments, the PEgRNA or nicking guide RNA comprises two and no more than two contiguous phosphorothioate linkages at the 5′ end or at the 3′ end. In some embodiments, the PEgRNA or nicking guide RNA comprises three contiguous phosphorothioate linkages at the 5′ end or at the 3′ end. In some embodiments, the PEgRNA or nicking guide RNA comprises the sequence 5′-UsUsU-3′ at the 3′end or at the 5′ end, wherein U indicates a uridine and wherein s indicates a phosphorothioate linkage. In some embodiments, the nucleobase may be chemically modified. Examples of chemical modifications to the nucleobase include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

Non-limiting examples of modifications may include 2′-O-methyl (2′-O-Me), 2′-O-(2-methoxyethyl) (2′-O-MOE), 2′-fluoro (2′-F), phosphorothioate (PS) bond between nucleotides, G-C substitutions, and inverted abasic linkages between nucleotides and equivalents thereof. In some embodiments, the PEgRNA comprises a chemical modification selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-5 propynyl-deoxycytidine (pdC), C-5 propynyl-deoxyuridine (pdU), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), 5-methyl m-cytidine, 5-methyl m-uridine, MGB (minor groove binder) pseudouridine (Ψ), 1-N-methylpseudouridine (1-Me-Ψ), 5-methoxyuridine (5-MO-U) 2′-O-methyl modifications, 2′-O-(2-methoxyethyl) modifications, 2′-fluoro modifications, phosphorothioate modifications, inverted abasic modifications, deoxyribonucleotides, bicylic ribose analog (e.g., locked nucleic acid (LNA), C-ethylene-bridged nucleic acid (ENA), bridged nucleic acid (BNA), unlocked nucleic acid (UNA)), base or nucleobase modifications, internucleoside linkage modifications, ribonebularine, 2′-O-methylnebularine, or 2′-deoxynebularine. Other examples of modifications include, but are not limited to, 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, abasic, acridine, azobenzene, biotin, biotin BB, biotin TEG, cholesteryl TEG, desthiobiotin, TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl-3-phosphorothioate or any combinations thereof.

In some embodiments, the PEgRNAs and/or nicking guide RNAs provided in this disclosure may have undergone modifications, e.g., chemical modifications or biological modifications. Modifications may be made at any position within a PEgRNA or nicking guide RNA, and may include one or more modifications to a nucleobase, a ribose component, a phosphate backbone, or any combinations thereof. In some embodiments, a modification can be a structure guided modification. In some embodiments, a modification is at the 5′ end and/or the 3′ end of a PEgRNA. In some embodiments, a chemical modification is at the 5′ end and/or the 3′ end of a nicking guide RNA. In some embodiments, a modification may be within the spacer sequence, the extension arm, the DNA synthesis template, and/or the primer binding site of a PEgRNA. In some embodiments, a modification may be within the spacer sequence or the gRNA core of a PEgRNA or a nicking guide RNA. In some embodiments, a modification may be within the 3′ most end of a PEgRNA or nicking guide RNA. In some embodiments, a modification may be within the 5′ most end of a PEgRNA or nicking guide RNA. In some embodiments, a PEgRNA or nicking guide RNA comprises 1, 2, 3, 4, 5, 6, 7, 8, 9, 10 or more modified nucleotides at the 3′ end. In some embodiments, a PEgRNA or nicking guide RNA comprises 1, 2, 3, 4, 5, 6, 7, 8, 9, 10 or more modified nucleotides at the 5′ end. In some embodiments, a PEgRNA or nicking guide RNA comprises 1, 2, 3, 4, or 5 or more modified nucleotides at the 3′ end. In some embodiments, a PEgRNA or nicking guide RNA comprises 1, 2, 3, 4, or 5 more modified nucleotides at the 5′ end. In some embodiments, a PEgRNA or nicking guide RNA comprises 1, 2, or 3 or more modified nucleotides at the 3′ end. In some embodiments, a PEgRNA or nicking guide RNA comprises 1, 2, or 3 more modified nucleotides at the 5′ end. In some embodiments, a PEgRNA or nicking guide RNA comprises 1, 2, 3, 4, 5, 6, 7, 8, 9, 10 or more contiguous modified nucleotides at the 3′ end. In some embodiments, a PEgRNA or nicking guide RNA comprises 1, 2, 3, 4, 5, 6, 7, 8, 9, 10 or more contiguous modified nucleotides at the 5′ end. In some embodiments, a PEgRNA or nicking guide RNA comprises 1, 2, 3, 4, or 5 contiguous modified nucleotides at the 3′ end. In some embodiments, a PEgRNA or nicking guide RNA comprises 1, 2, 3, 4, or 5 contiguous modified nucleotides at the 5′ end. In some embodiments, a PEgRNA or nicking guide RNA comprises 1, 2, or 3 contiguous modified nucleotides at the 3′ end. In some embodiments, a PEgRNA or nicking guide RNA comprises 1, 2, or 3 contiguous modified nucleotides at the 5′ end. In some embodiments, a PEgRNA or nicking guide RNA comprises 3 contiguous modified nucleotides at the 3′ end. In some embodiments, a PEgRNA or nicking guide RNA comprises 1, 2, 3, 4, 5, or more modified nucleotides near the 3′ end. In some embodiments, a PEgRNA or nicking guide RNA comprises 3 contiguous modified nucleotides at the 3′ end. In some embodiments, a PEgRNA or nicking guide RNA comprises 3 contiguous modified nucleotides at the 5′ end. In some embodiments, a PEgRNA or nicking guide RNA comprises 1, 2, 3, 4, 5, or more modified nucleotides near the 3′ end. In some embodiments, a PEgRNA or nicking guide RNA comprises 1, 2, 3, 4, 5, or more contiguous modified nucleotides near the 3′ end.

pegRNA Design Method

The present disclosure also relates to methods for designing pegRNAs.

In one aspect of design, the design approach can take into account the particular application for which prime editing is being used. For instance, and as exemplified and discussed herein, prime editing can be used, without limitation, to (a) install mutation-correcting changes to a nucleotide sequence, (b) install protein and RNA tags, (c) install immunoepitopes on proteins of interest, (d) install inducible dimerization domains in proteins, (e) install or remove sequences to alter that activity of a biomolecule, (f) install recombinase target sites to direct specific genetic changes, and (g) mutagenesis of a target sequence by using an error-prone RT. In addition to these methods which, in general, insert, change, or delete nucleotide sequences at target sites of interest, prime editors can also be used to construct highly programmable libraries, as well as to conduct cell data recording and lineage tracing studies. In these various uses, there may be as described herein particular design aspects pertaining to the preparation of a pegRNA that is particularly useful for any given of these applications.

When designing a pegRNA for any particular application or use of prime editing, a number of considerations may be taken into account, which include, but are not limited to:
(a) the target sequence, i.e., the nucleotide sequence in which one or more nucleobase modifications are desired to be installed by the prime editor;
(b) the location of the cut site within the target sequence, i.e., the specific nucleobase position at which the prime editor will induce a single-stand nick to create a 3′ end RT primer sequence on one side of the nick and the 5′ end endogenous flap on the other side of the nick (which ultimately is removed by FEN1 or equivalent thereto and replaced by the 3′ ssDNA flap. The cut site is analogous to the “edit location” since this what creates the 3′ end RT primer sequence which becomes extended by the RT during RNA-depending DNA polymerization to create the 3′ ssDNA flap containing the desired edit, which then replaces the 5′ endogenous DNA flap in the target sequence.
(c) the available PAM sequences (including the canonical SpCas9 PAM sites, as well as non-canonical PAM sites recognized by Cas9 variants and equivalents with expanded or differing PAM specificities);
(d) the spacing between the available PAM sequences and the location of the cut site in the target sequence;
(e) the particular Cas9, Cas9 variant, or Cas9 equivalent of the prime editor being used;
(f) the sequence and length of the primer binding site;
(g) the sequence and length of the edit template;
(h) the sequence and length of the homology arm;
(i) the spacer sequence and length; and
(j) the core sequence.

The instant disclosure discusses these aspects above.

In one embodiment, an approach to designing a suitable pegRNA, and optionally a nicking-sgRNA design guide for second-site nicking, is hereby provided. This embodiment provides a step-by-step set of instructions for designing pegRNAs and nicking-sgRNAs for prime editing which takes into account one or more of the above considerations. The steps reference the examples shown in FIGS. 70A-70I.
1. Define the target sequence and the edit. Retrieve the sequence of the target DNA region (˜200 bp) centered around the location of the desired edit (point mutation, insertion, deletion, or combination thereof). See FIG. 70A.
2. Locate target PAMs. Identify PAMs in the proximity to the desired edit location. PAMs can be identified on either strand of DNA proximal to the desired edit location. While PAMs close to the edit position are preferred (i.e., wherein the nick site is less than 30 nt from the edit position, or less than 29 nt, 28 nt, 27 nt, 26 nt, 25 nt, 24 nt, 23 nt, 22 nt, 21 nt, 20 nt, 19 nt, 18 nt, 17 nt, 16 nt, 15 nt, 14 nt, 13 nt, 12 nt, 11 nt, 10 nt, 9 nt, 8 nt, 7 nt, 6 nt, 5 nt, 4 nt, 3 nt, or 2 nt from the edit position to the nick site), it is possible to install edits using protospacers and PAMs that place the nick ≥30 nt from the edit position. See FIG. 70B.
3. Locate the nick sites. For each PAM being considered, identify the corresponding nick site and on which strand. For Sp Cas9 H840A nickase, cleavage occurs in the PAM-containing strand between the 3^{rd} and 4^{th} bases 5′ to the NGG PAM. All edited nucleotides must exist 3′ of the nick site, so appropriate PAMs must place the nick 5′ to the target edit on the PAM-containing strand. In the example shown below, there are two possible PAMs. For simplicity, the remaining steps will demonstrate the design of a pegRNA using PAM 1 only. See FIG. 70C.
4. Design the spacer sequence. The protospacer of Sp Cas9 corresponds to the 20 nucleotides 5′ to the NGG PAM on the PAM-containing strand. Efficient Pol III transcription initiation requires a G to be the first transcribed nucleotide. If the first nucleotide of the protospacer is a G, the spacer sequence for the pegRNA is simply the protospacer sequence. If the first nucleotide of the protospacer is not a G, the spacer sequence of the pegRNA is G followed by the protospacer sequence. See FIG. 70D.
5. Design a primer binding site (PBS). Using the starting allele sequence, identify the DNA primer on the PAM-containing strand. The 3′ end of the DNA primer is the nucleotide just upstream of the nick site (i.e. the 4^{th} base 5′ to the NGG PAM for Sp Cas9). As a general design principle for use with PE2 and PE3, a pegRNA primer binding site (PBS) containing 12 to 13 nucleotides of complementarity to the DNA primer can be used for sequences that contain ˜40-60% GC content. For sequences with low GC content, longer (14- to 15-nt) PBSs should be tested. For sequences with higher GC content, shorter (8- to 11-nt) PBSs should be tested. Optimal PBS sequences should be determined empirically, regardless of GC content. To design a length-p PBS sequence, take the reverse complement of the first p nucleotides 5′ of the nick site in the PAM-containing strand using the starting allele sequence. See FIG. 70E.
6. Design an RT template (or DNA synthesis template). The RT template (or DNA synthesis template where the polymerase is not reverse transcriptase) encodes the designed edit and homology to the sequence adjacent to the edit. In one embodiment, these regions correspond to the DNA synthesis template of FIG. 3D and FIG. 3E, wherein the DNA synthesis template comprises the “edit template” and the “homology arm.” Optimal RT template lengths vary based on the target site. For short-range edits (positions +1 to +6), it is recommended to test a short (9 to 12 nt), a medium (13 to 16 nt), and a long (17 to 20 nt) RT template. For long-range edits (positions +7 and beyond), it is recommended to use RT templates that extend at least 5 nt (preferably 10 or more nt) past the position of the edit to allow for sufficient 3′ DNA flap homology. For long-range edits, several RT templates should be screened to identify functional designs. For larger insertions and deletions (≥5 nt), incorporation of greater 3′ homology (˜20 nt or more) into the RT template is recommended. Editing efficiency is typically impaired when the RT template encodes the synthesis of a G as the last nucleotide in the reverse transcribed DNA product (corresponding to a C in the RT template of the pegRNA). As many RT templates support efficient prime editing, avoidance of G as the final synthesized nucleotide is recommended when designing RT templates. To design a length-r RT template sequence, use the desired allele sequence and take the reverse complement of the first r nucleotides 3′ of the nick site in the strand that originally contained the PAM. Note that compared to SNP edits, insertion or deletion edits using RT templates of the same length will not contain identical homology. See FIG. 70F.
7. Assemble the full pegRNA sequence. Concatenate the pegRNA components in the following order (5′ to 3′): spacer, scaffold, RT template and PBS. See FIG. 70G.
8. Designing nicking-sgRNAs for PE3. Identify PAMs on the non-edited strand upstream and downstream of the edit. Optimal nicking positions are highly locus-dependent and should be determined empirically. In general, nicks placed 40 to 90 nucleotides 5′ to the position across from the pegRNA-induced nick lead to higher editing yields and fewer indels. A nicking sgRNA has a spacer sequence that matches the 20-nt protospacer in the starting allele, with the addition of a 5′-G if the protospacer does not begin with a G. See FIG. 70H.
9. Designing PE3b nicking-sgRNAs. If a PAM exists in the complementary strand and its corresponding protospacer overlaps with the sequence targeted for editing, this edit could be a candidate for the PE3b system. In the PE3b system, the spacer sequence of the nicking-sgRNA matches the sequence of the desired edited allele, but not the starting allele. The PE3b system operates efficiently when the edited nucleotide(s) falls within the seed region (˜10 nt adjacent to the PAM) of the nicking-sgRNA protospacer. This prevents nicking of the complementary strand until after installation of the edited strand, preventing competition between the pegRNA and the sgRNA for binding the target DNA. PE3b also avoids the generation of simultaneous nicks on both strands, thus reducing indel formation significantly while maintaining high editing efficiency. PE3b sgRNAs should have a spacer sequence that matches the 20-nt protospacer in the desired allele, with the addition of a 5′ G if needed. See FIG. 70I.

The above step-by-step process for designing a suitable pegRNA and a second-site nicking sgRNA is not meant to be limiting in any way. The disclosure contemplates variations of the above-described step-by-step process which would be derivable therefrom by a person of ordinary skill in the art. pegRNA modifications

The present disclosure provides next-generation modified pegRNAs with improved properties, including but not limited to, increased stability and cellular lifespan, and improved binding affinity for a napDNAbp. These modified pegRNAs result in improved genome editing as demonstrated by increase editing efficiency at a wide variety of genomic sites. The present inventors have surprisingly found that by appending certain nucleic acid structural motifs to terminus of the extension arm of a pegRNA, including but limited to, a prequeosin₁-1 riboswitch aptamer (“evopreQ₁-1”) or variant thereof, a pseudoknot from the MMLV viral genome (“evopreQ₁-1”) or variant thereof, a modified tRNA used by MMLV RT as a primer for reverse transcription or variant thereof, and a G quadruplex or variant thereof, a consistent increase in editing activity was achieved.

In one embodiment, the modified pegRNAs include a nucleic acid moiety at the 3′ end of the pegRNA in accordance with FIG. 98. Optionally, the 3′ end of the pegRNA is fused to the nucleic acid moiety through a nucleotide linker. In various embodiments, it will be appreciated that a wide variety of nucleotide sequences will work reasonably well for each genomic target site. Linker length can also be variable. In some cases, linkers ranging in length from 3-18 nucleotides will work. In other cases, the linker may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In general, the nucleic acid moieties that may be used to modify a pegRNA, for example, by attaching it to the 3′ end of a pegRNA, may include any nucleic acid moiety, including, for instance, a nucleic acid molecule comprising or which forms a double-helix moiety, toeloop moiety, hairpin moiety, stem-loop moiety, pseudoknot moiety, aptamer moiety, G quadraplex moiety, tRNA moiety, or a ribozyme moiety. The nucleic acid moiety may be characterized as forming a secondary nucleic acid structure, a tertiary nucleic acid structure, or a quadruple nucleic acid structure. In other words, the nucleic acid moiety may form any two dimensional or three dimensional structure known to be formed by such structures. The nucleic acid moiety may be DNA or RNA.

Without restriction, the following are specific examples of nucleotide motifs that may be appended to the terminus of the extension arm of a pegRNA. Thus, in the case of a ′3 extension arm, the nucleotide motif would be coupled, attached, or otherwise linked to the 3′ of the pegRNA, optionally via a linker. In the case of a 5′ extension arm, the nucleotide motif would be coupled, attached, or otherwise linked to the 5′ end of the pegRNA, optionally via a linker.

TABLE 4
Mpknot 1 and variants

| | | |
|---|---|---|
| Mpknot1 | GGGTCAGGAGCCCCCCCCCTGAACCCAGGAT | SEQ ID NO: 195 |
| | AACCCTCAAAGTCGGGGGGCAACCC | |
| Mpknot1 3′ | GGGTCAGGAGCCCCCCCCCTGAACCCAGGAT | SEQ ID NO: 196 |
| trimmed | AACCCTCAAAGTCGGGGGGC | |
| Mpknot1 with 5′ | GTCAGGGTCAGGAGCCCCCCCCCTGAACCCA | SEQ ID NO: 197 |
| extra | GGATAACCCTCAAAGTCGGGGGGCAACCC | |
| Mpknot1 U38A | GGGTCAGGAGCCCCCCCCCTGAACCCAGGAA | SEQ ID NO: 198 |
| | AACCCTCAAAGTCGGGGGGCAACCC | |
| Mpknot1 U38A | GGGTCAGGAGCCCCCCCCCTGCACCCAGGAA | SEQ ID NO: 199 |
| A29C | AACCCTCAAAGTCGGGGGGCAACCC | |
| MMLC A29C | GGGTCAGGAGCCCCCCCCCTGCACCCAGGAT | SEQ ID NO: 200 |
| | AACCCTCAAAGTCGGGGGGCAACCC | |
| Mpknot1 with 5′ | GTCAGGGTCAGGAGCCCCCCCCCTGAACCCA | SEQ ID NO: 201 |
| extra and U38A | GGAAAACCCTCAAAGTCGGGGGGCAACCC | |
| Mpknot1 with 5′ | GTCAGGGTCAGGAGCCCCCCCCCTGCACCCA | SEQ ID NO: 202 |
| extra and U38A | GGAAAACCCTCAAAGTCGGGGGGCAACCC | |
| A29C | | |
| Mpknot1 with 5′ | GTCAGGGTCAGGAGCCCCCCCCCTGCACCCA | SEQ ID NO: 203 |
| extra and A29C | GGATAACCCTCAAAGTCGGGGGGCAACCC | |

TABLE 5
G quadruplexes

| | | |
|---|---|---|
| tns1 | Gggctgggatgggaaaggg | SEQ ID NO: 204 |
| stk40 | Gggacagggcagggacaggg | SEQ ID NO: 205 |
| apc2 | Gggtccgggtctgggtctggg | SEQ ID NO: 206 |
| ceacam4 | Gggctctgggtgggccggg | SEQ ID NO: 207 |
| pitpnm3 | Gggtgggctgggaaggg | SEQ ID NO: 208 |
| rlf | Gggagggagggctaggg | SEQ ID NO: 209 |
| erc1 | Gggctgggctgggcaggg | SEQ ID NO: 210 |
| ube3c | Gggcagggctgggaggg | SEQ ID NO: 211 |
| taf15 | Gggtgggagggctggg | SEQ ID NO: 212 |
| stard3 | Gggcagggtctgggctggg | SEQ ID NO: 213 |
| g2 | Tggtggtggtgg | SEQ ID NO: 214 |

TABLE 6
evopreql and similar/variant motifs

| | | |
|---|---|---|
| evopreq1 | TTGACGCGGTTCTATCTAGTTACGCGTTA | SEQ ID NO: |
| | AACCAACTAGAAA | 215 |
| evopreq1 | CGCGAGTCTAGGGGATAACGCGTTAAACT | SEQ ID NO: |
| motif1 | TCCTAGAAGGCGGTT | 216 |
| evopreq1 | CGCGGATCTAGATTGTAACGCGTTAAACC | SEQ ID NO: |
| motif2 | ATCTAGAAGGCGGTT | 217 |
| evopreq1 | CGCGTCGCTACCGCCCGGCGCGTTAAACA | SEQ ID NO: |
| motif3 | CACTAGAAGGCGGTT | 218 |
| shorter | CGCGGTTCTATCTAGTTACGCGTTAAACC | SEQ ID NO: |
| preq1-1 | AACTAGAA | 219 |
| preq1-1 | TTGACGCGCTTCTATCTAGTTACGCGTTA | SEQ ID NO: |
| G5C | AACCAACTAGAAA | 220 |
| (mut1) | | |
| preq1-1 | TTGACGCGGTTCTATCTACTTACGCGTTA | SEQ ID NO: |
| G15C | AACCAACTAGAAA | 221 |
| (mut2) | | |

TABLE 7
Modified tRNA that is used by MMLV RT as
a primer for reverse transcription

GGCGGGGCTCGTTGGTCTAGGGGTATGATTCTCGCTTCGGGTGCGAGAGG
TCCCGGGTTCAAATCCCGGACGAGCCCCGCC (SEQ ID NO: 222)

TABLE 8
Miscellaneous motifs

xrn1 - gcgtaacctccatccgagttgcaagagagggaaacgcagtctc
(SEQ ID NO: 223)
grp1 intron P4P6 - ggaattgcgggaaaggggtcaacagccgttc
agtaccaagtctcaggggaaactttgagatggccttgcaaagggtatggt
aataagctgacggacatggtcctaaccacgcagccaagtcctaagtcaac
agatcttctgttgatatggatgcagttca (SEQ ID NO: 224)

As indicated above, these motifs may be couples, attached, or otherwise joined to a canonical pegRNA via a linker. Exemplary linkers include, but are not limited to:

TABLE X6
Linkers for joining motifs to canonical pegRNA

| Genomic | | | Length |
|---|---|---|---|
| locus | Linker sequence | SEQ ID NO: | (nts) |
| HEK3 | TCTCTCTC | SEQ ID NO: 225 | 8 |
| HEK3 | TCTCTCTCACACACACAC | SEQ ID NO: 226 | 18 |
| RNF2 | TCATCTCT | SEQ ID NO: 227 | 8 |
| RNF2 | TCATCTCTACACACACAC | SEQ ID NO: 228 | 18 |
| FANCF | CAATCACT | SEQ ID NO: 229 | 8 |
| FANCF | CAATCACTACACACACAC | SEQ ID NO: 230 | 18 |
| RUNX1 | AACTCTCT | SEQ ID NO: 231 | 8 |
| RUNX1 | AACTCTCTACACACACAC | SEQ ID NO: 232 | 18 |
| EMX1 | AACAATCT | SEQ ID NO: 233 | 8 |
| EMX1 | AACAATCTACACACACAC | SEQ ID NO: 234 | 18 |
| DNMT1 | CCTCTTCT | SEQ ID NO: 235 | 8 |
| DNMT1 | CCTCTTCTACACACACAC | SEQ ID NO: 236 | 18 |

In some embodiments, a linker will be designed and/or selected based on the genomic site being targeted by prime editing and the modified pegRNA.

In various embodiments, it will be appreciated that a wide variety of nucleotide sequences will work reasonably well for each genomic target site. Linker length is also likely to be variable. In some cases, linkers ranging in length from 3-18 nucleotides will work. In other cases, the linker may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In one embodiment, the linker is 8 nucleotides in length.

The present disclosure also contemplates variants of the above nucleotide motifs and linkers which have at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity with any of the above motif and linker sequences.

The pegRNAs may also include additional design improvements that may modify the properties and/or characteristics of pegRNAs thereby improving the efficacy of prime editing. In various embodiments, these improvements may belong to one or more of a number of different categories, including but not limited to: (1) designs to enable efficient expression of functional pegRNAs from non-polymerase III (pol III) promoters, which would enable the expression of longer pegRNAs without burdensome sequence requirements; (2) improvements to the core, Cas9-binding pegRNA scaffold, which could improve efficacy; (3) modifications to the pegRNA to improve RT processivity, enabling the insertion of longer sequences at targeted genomic loci; and (4) addition of RNA motifs to the 5′ or 3′ termini of the pegRNA that improve pegRNA stability, enhance RT processivity, prevent misfolding of the pegRNA, or recruit additional factors important for genome editing.

In one embodiment, pegRNA could be designed with polIII promoters to improve the expression of longer-length pegRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed pegRNA, which has been shown to result in markedly reduced Cas9:sgRNA activity in a site-dependent manner. Additionally, while pol III-transcribed pegRNAs can simply terminate in a run of 6-7 U's, pegRNAs transcribed from pol II or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Exemplary U6 promoters include, but are not limited to:

U6 promoter:
(SEQ ID NO: 237)
GAGGGCCTATTTCCCATGATTCCTTCATATTTGCATATACGATACAAGGC
TGTTAGAGAGATAATTAGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACCG
U6v9 promoter:
(SEQ ID NO: 238)
GCCTGAGGCGTGGGGCCGCCTCCCAAAGACTTCTGGGAGGGCGGTGCGGC
TCAGGCTCTGCCCCGCCTCCGGGGCTATTTGCATACGACCATTTCCAGTA
ATTCCCAGCAGCCACCGTAGCTATATTTGGTAGAACAACGAGCACTTTCT
CAACTCCAGTCAATAACTACGTTAGTTGCATTACACATTGGGCTAATATA
AATAGAGGTTAAATCTCTAGGTCATTTAAGAGAAGTCGGCCTATGTGTAC
AGACATTTGTTCCAGGGGCTTTAAATAGCTGGTGGTGGAACTCAATATTC
G
U6v7 promoter:
(SEQ ID NO: 239)
AAGTCCGCGGCACGAGAAATCAAAGCCCCGGGGCCTGGGTCCCACGCGGG
GTCCCTTACCCAGGGTGCCCCGGGCGCTCATTTGCATGTCCCACCCAACA
GGTAAACCTGACAGATCGGTCGCGGCCAGGTACGGCCTGGCGGTCAGAGC
ACCAAACTTACGAGCCTTGTGATGAGTTCCGTTACATGAAATTCTCCTAA
AGGCTCCAAGATGGACAGGAAAGCGCTCGATTAGGTTACCGTAAGGAAAA
CAAATGAGAAACTCCCGTGCCTTATAAGACCTGGGGACGGACTTATTTGC
G
U6v4 promoter:
(SEQ ID NO: 240)
AAATTGAGTCATCTGACAGAAATTATCTTTGGCAAGGTTTTAGTCCTAGG
GTTACCAGATGGAATACAGGACATCCATTTAAATTTGAATTTCAGATAAA
CAGTTAACACTTCTCAAGGATAAATATGCCTCAAATATTGCACGGGACAT
ATTTATACTAAAAAAAAAGTGTTTTTTTTTTTCCTGCGATTCAAACTTAA
CTGGTGTCCTGCATTTGTATTTGTTAAATCTGTCAATCCTATCTCAGTTT
CCTTTGATGGAATGTACCTCTGTGCTAATATTTAAAAATAGGTTACATTT
G

One of ordinary skill in the art will appreciate that these promoter sequences can be trimmed at the 5′ and still function at the same or nearly the same level. For example, any of the U6 promoters could be trimmed at the 5′ end by removing up to 1, 10, 20, 30, 40, 50, 60, 70, 80, 90, or 100 nucleotides from the 5′end, i.e., approximately 30% of the promoter length. In other embodiments, up to 1%, 2%, 3%, 4%, 5%, 6%, 7%, 8%, 9%, 10%, 11%, 12%, 13%, 14%, 15%, 16%, 17%, 18%, 19%, 19%, 20%, 21%, 22%, 23%, 24%, 25%, 26%, 27%, 28%, 29%, or up to 30% of the length of the promoter from the 5′ end.

One of ordinary skill in the art will also appreciate that other promoters could be used to improve the expression of longer length pegRNAs with larger extension arms. For example, in different cell types, other promoters may be preferred and result in greater expression of the longer length pegRNAs.

Previously, Rinn and coworkers screened a variety of expression platforms for the production of long-noncoding RNA- (lncRNA) tagged sgRNAs¹⁸³. These platforms include RNAs expressed from pCMV and that terminate in the ENE element from the MALAT1 ncRNA from humans¹⁸⁴, the PAN ENE element from KSHViss, or the 3′ box from U1 snRNA¹⁸⁶. Notably, the MALAT1 ncRNA and PAN ENEs form triple helices protecting the polyA-tail^{184, 187}. These constructs could also enhance RNA stability. It is contemplated that these expression systems will also enable the expression of longer pegRNAs.

In addition, a series of methods have been designed for the cleavage of the portion of the pol II promoter that would be transcribed as part of the pegRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy4¹⁹³ and also leads to processing of the guide. Also, incorporation of multiple ENE motifs can lead to improved pegRNA expression and stability. Circularizing, as previously demonstrated for the KSHV PAN RNA and element¹⁸⁵. It is also anticipated that circularizing the pegRNA in the form of a circular intronic RNA (ciRNA) can lead to enhanced RNA expression and stability, as well as nuclear localization.

In various embodiments, the pegRNA may include various above elements, as exemplified by the following sequence.

Non-limiting example 1—pegRNA expression platform consisting of pCMV, Csy4 hairpin, the pegRNA, and MALAT1 ENE

(SEQ ID NO: 241)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTAGGGTCATGAAGGTT
TTTCTTTTCCTGAGAAAACAACACGTATTGTTTTCTCAGGTTTTGCTTTT
TGGCCTTTTTCTAGCTTAAAAAAAAAAAAAGCAAAAGATGCTGGTGGTTG
GCACTCCTGGTTTCCAGGACGGGGTTCAAATCCCTGCGGCGTCTTTGCTT
TGACT

Non-limiting example 2—pegRNA expression platform consisting of pCMV, Csy4 hairpin, the pegRNA, and PAN ENE

(SEQ ID NO: 242)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAA

Non-limiting example 3—pegRNA expression platform consisting of pCMV, Csy4 hairpin, the pegRNA, and 3×PAN ENE

(SEQ ID NO: 243)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAAACACACTG
TTTTGGCTGGGTTTTTCCTTGTTCGCACCGGACACCTCCAGTGACCAGAC
GGCAAGGTTTTTATCCCAGTGTATATTGGAAAAACATGTTATACTTTTGA
CAATTTAACGTGCCTAGAGCTCAAATTAAACTAATACCATAACGTAATGC
AACTTACAACATAAATAAAGGTCAATGTTTAATCCATAAAAAAAAAAAAA
AAAAAATCTCTCTGTTTTGGCTGGGTTTTTCCTTGTTCGCACCGGACACC
TCCAGTGACCAGACGGCAAGGTTTTTATCCCAGTGTATATTGGAAAAACA
TGTTATACTTTTGACAATTTAACGTGCCTAGAGCTCAAATTAAACTAATA
CCATAACGTAATGCAACTTACAACATAAATAAAGGTCAATGTTTAATCCA
TAAAAAAAAAAAAAAAAAAA

Non-limiting example 4—pegRNA expression platform consisting of pCMV, Csy4 hairpin, the pegRNA, and 3′ box

(SEQ ID NO: 244)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTGTTTCAAAAGTAGACT
GTACGCTAAGGGTCATATCTTTTTTTGTTTGGTTTGTGTCTTGGTTGGCG
TCTTAAA

Non-limiting example 5—pegRNA expression platform consisting of pU1, Csy4 hairpin, the pegRNA, and 3′ box

(SEQ ID NO: 245)
CTAAGGACCAGCTTCTTTGGGAGAGAACAGACGCAGGGGCGGGAGGGAAA
AAGGGAGAGGCAGACGTCACTTCCCCTTGGCGGCTCTGGCAGCAGATTGG
TCGGTTGAGTGGCAGAAAGGCAGACGGGGACTGGGCAAGGCACTGTCGGT
GACATCACGGACAGGGCGACTTCTATGTAGATGAGGCAGCGCAGAGGCTG
CTGCTTCGCCACTTGCTGCTTCACCACGAAGGAGTTCCCGTGCCCTGGGA
GCGGGTTCAGGACCGCTGATCGGAAGTGAGAATCCCAGCTGTGTGTCAGG
GCTGGAAAGGGCTCGGGAGTGCGCGGGGCAAGTGACCGTGTGTGTAAAGA
GTGAGGCGTATGAGGCTGTGTCGGGGCAGAGGCCCAAGATCTCAGTTCAC
TGCCGTATAGGCAGGGCCCAGACTGAGCACGTGAGTTTTAGAGCTAGAAA
TAGCAAGTTAAAATAAGGCTAGTCCGTTATCAACTTGAAAAAGTGGGACC
GAGTCGGTCCTCTGCCATCAAAGCGTGCTCAGTCTGTTTCAGCAAGTTCA
GAGAAATCTGAACTTGCTGGATTTTTGGAGCAGGGAGATGGAATAGGAGC
TTGCTCCGTCCACTCCACGCATCGACCTGGTATTGCAGTACCTCCAGGAA
CGGTGCACCCACTTTCTGGAGTTTCAAAAGTAGACTGTACGCTAAGGGTC
ATATCTTTTTTTGTTTGGTTTGTGTCTTGGTTGGCGTCTTAAA.

In various other embodiments, the pegRNA may be improved by introducing improvements to the scaffold or core sequences.

The core, Cas9-binding pegRNA scaffold can be improved to enhance PE activity. In an exemplary approach, the first pairing element of the scaffold (P1) contains a GTTTT-AAAAC (SEQ ID NO: 246) pairing element. Such runs of Ts can result in pol III pausing and premature termination of the RNA transcript. Rational mutation of one of the T-A pairs to a G-C pair in this portion of P1 can enhance sgRNA activity. This approach can be used to improve pegRNAs. Additionally, increasing the length of P1 can enhance sgRNA folding and lead to improved pegRNA activity. Example improvements to the core can include:

pegRNA containing a 6 nt extension to P1
(SEQ ID NO: 247)
GGCCCAGACTGAGCACGTGAGTTTTAGAGCTAGCTCATGAAAATGAGCTA
GCAAGTTAAAATAAGGCTAGTCCGTTATCAACTTGAAAAAGTGGGACCGA
GTCGGTCCTCTGCCATCAAAGCGTGCTCAGTCTGTTTTTTT
pegRNA containing a T-A to G-C mutation
within P1
(SEQ ID NO: 248)
GGCCCAGACTGAGCACGTGAGTTTGAGAGCTAGAAATAGCAAGTTTAAAT
AAGGCTAGTCCGTTATCAACTTGAAAAAGTGGGACCGAGTCGGTCCTCTG
CCATCAAAGCGTGCTCAGTCTGTTTTTTT
pegRNA split into CRISPR- and tracrRNA
components:
(SEQ ID NO: 249)
GGCCCAGACTGAGCACGTGAGTTTTAGAGCTAGA
(SEQ ID NO: 250)
AATAGCAAGTTAAAATAAGGCTAGTCCGTTATCAACTTGAAAAAGTGGGA
CCGAGTCGGTCCTCTGCCATCAAAGCGTGCTCAGTCTG

In various other embodiments, the pegRNA may be improved by introducing modifications to the edit template region. As the size of the insertion templated by the pegRNA increases, it is more likely to be degraded by endonucleases, undergo spontaneous hydrolysis, or fold into secondary structures unable to be reverse-transcribed by the RT or that disrupt folding of the pegRNA scaffold and subsequent Cas9-RT binding. Accordingly, it is likely that modification to the template of the pegRNA might be necessary to affect large insertions, such as the insertion of whole genes. Some strategies to do so include the incorporation of modified nucleotides within a synthetic or semi-synthetic pegRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the pegRNA to enhance stability and activity. Alternatively or additionally, the template of the pegRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate pegRNAs. In such a design, a PE would be used to initiate transcription and also recruit a separate template RNA to the targeted site via an RNA-binding protein fused to Cas9 or an RNA recognition element on the pegRNA itself such as the MS2 aptamer. The RT could either directly bind to this separate template RNA, or initiate reverse transcription on the original pegRNA before swapping to the second template. Such an approach could enable long insertions by both preventing misfolding of the pegRNA upon addition of the long template and also by not requiring dissociation of Cas9 from the genome for long insertions to occur, which could possibly be inhibiting PE-based long insertions.

In still other embodiments, the pegRNA may be improved by introducing additional RNA motifs at the 5′ and 3′ termini of the pegRNAs, or even at positions therein between (e.g., in the gRNA core region, or the spacer). Several such motifs—such as the PAN ENE from KSHV and the ENE from MALAT1 were discussed above as possible means to terminate expression of longer pegRNAs from non-pol III promoters. These elements form RNA triple helices that engulf the polyA tail, resulting in their being retained within the nucleus^{184,187} However, by forming complex structures at the 3′ terminus of the pegRNA that occlude the terminal nucleotide, these structures would also likely help prevent exonuclease-mediated degradation of pegRNAs.

Other structural elements inserted at the 3′ terminus could also enhance RNA stability, albeit without enabling termination from non-pol III promoters. Such motifs could include hairpins or RNA quadruplexes that would occlude the 3′ terminus¹⁹⁷, or self-cleaving ribozymes such as HDV that would result in the formation of a 2′-3′-cyclic phosphate at the 3′ terminus and also potentially render the pegRNA less likely to be degraded by exonucleases¹⁹⁸. Inducing the pegRNA to cyclize via incomplete splicing—to form a ciRNA—could also increase pegRNA stability and result in the pegRNA being retained within the nucleus¹⁹⁴

Additional RNA motifs could also improve RT processivity or enhance pegRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹.

Addition of dimerization motifs—such as kissing loops or a GNRA tetraloop/tetraloop receptor pair²⁰⁰—at the 5′ and 3′ termini of the pegRNA could also result in effective circularization of the pegRNA, improving stability. Additionally, it is envisioned that addition of these motifs could enable the physical separation of the pegRNA spacer and primer, preventing prevention occlusion of the spacer which could would hinder PE activity. Short 5′ extensions or 3′ extensions to the pegRNA that form a small toehold hairpin in the spacer region or along the primer binding site could also compete favorably against the annealing of intracomplementary regions along the length of the pegRNA, e.g., the interaction between the spacer and the primer binding site that can occur. Finally, kissing loops could also be used to recruit other template RNAs to the genomic site and enable swapping of RT activity from one RNA to the other. As exemplary embodiments of various secondary structures, the pegRNA depicted in FIG. 3D and FIG. 3E list a number secondary RNA structures that may be engineered into any region of the pegRNA, including in the terminal portions of the extension arm (i.e., eland e2), as shown.

Example improvements include, but are not limited to:

pegRNA-HDV fusion
(SEQ ID NO: 251)
GGCCCAGACTGAGCACGTGAGTTTTAGAGCTAGAAATAGCAAGTTAAAAT
AAGGCTAGTCCGTTATCAACTTGAAAAAGTGGGACCGAGTCGGTCCTCTG
CCATCAAAGCGTGCTCAGTCTGGGCCGGCATGGTCCCAGCCTCCTCGCTG
GCGCCGGCTGGGCAACATGCTTCGGCATGGCGAATGGGACTTTTTTT
pegRNA-MMLV kissing loop
(SEQ ID NO: 252)
GGTGGGAGACGTCCCACCGGCCCAGACTGAGCACGTGAGTTTTAGAGCTA
GAAATAGCAAGTTAAAATAAGGCTAGTCCGTTATCAACTTGAAAAAGTGG
GACCGAGTCGGTCCTCTGCCATCAAAGCTTCGACCGTGCTCAGTCTGGTG
GGAGACGTCCCACCTTTTTTT
pegRNA-VS ribozyme kissing loop
(SEQ ID NO: 253)
GAGCAGCATGGCGTCGCTGCTCACGGCCCAGACTGAGCACGTGAGTTTTA
GAGCTAGAAATAGCAAGTTAAAATAAGGCTAGTCCGTTATCAACTTGAAA
AAGTGGGACCGAGTCGGTCCTCTGCCATCAAAGCTTCGACCGTGCTCAGT
CTCCATCAGTTGACACCCTGAGGTTTTTTT
pegRNA-GNRA tetraloop/tetraloop receptor
(SEQ ID NO: 254)
GCAGACCTAAGTGGUGACATATGGTCTGGGCCCAGACTGAGCACGTGAGT
TTTAGAGCTAUACGTAGCAAGTTAAAATAAGGCTAGTCCGTTATCAACTT
UACGAAGTGGGACCGAGTCGGTCCTCTGCCATCAAAGCTTCGACCGTGCT
CAGTCTGCATGCGATTAGAAATAATCGCATGTTTTTTT
pegRNA template switching secondary
RNA-HDV fusion
(SEQ ID NO: 255)
TCTGCCATCAAAGCTGCGACCGTGCTCAGTCTGGTGGGAGACGTCCCACC
GGCCGGCATGGTCCCAGCCTCCTCGCTGGCGCCGGCTGGGCAACATGCTT
CGGCATGGCGAATGGGACTTTTTTT

pegRNA scaffold could be further improved via directed evolution, in an analogous fashion to how SpCas9 and prime editor (PE) have been improved. Directed evolution could enhance pegRNA recognition by Cas9 or evolved Cas9 variants. Additionally, it is likely that different pegRNA scaffold sequences would be optimal at different genomic loci, either enhancing PE activity at the site in question, reducing off-target activities, or both. Finally, evolution of pegRNA scaffolds to which other RNA motifs have been added would almost certainly improve the activity of the fused pegRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-pegRNA fusions as well. In addition, while Cas9 currently does not generally tolerate 5′ extension of the sgRNA, directed evolution will likely generate enabling mutations that mitigate this intolerance, allowing additional RNA motifs to be utilized.

In various embodiments, other scaffolds that have been shown to improve activity relative to canonical sgRNA scaffolds may be used in pegRNAs and epegRNAs as described herein. Such improvements may include, for example, those disclosed in Chen, B. et al. Dynamic Imaging of Genomic Loci in Living Human Cells by an Optimized CRISPR/Cas System. Cell. 2013, 155(7), 1479-1471 and Jost, M. et al. Titrating expression using libraries of systematically attenuated CRISPR guide RNAs. Nat. Biotechnol. 2020, 38, 355-364, which are herein incorporated by reference in their entirety. These improvements may enhance epegRNA activity through improved binding to the prime editor and/or improved expression. Stabilization of the sgRNA scaffold could also reduce PBS/spacer interactions that inhibit pegRNA and epegRNA activity.

Example epegRNAs incorporating improved sgRNA scaffolds include, but are not limited to:

HEK3 1-15del standard scaffold evopreQ1
(SEQ ID NO: 256)
GGCCCAGACTGAGCACGTGAGTTTTAGAGCTAGAAATAGCAAGTTAAAAT
AAGGCTAGTCCGTTATCAACTTGAAAAAGTGGCACCGAGTCGGTGCGCCC
TCTGGAGGAAGCAGGGCTTCCCGTGCTCAGTCTGTCTCTCTCTTGACGCG
GTTCTATCTAGTTACGCGTTAAACCAACTAGAAATTTTTT
HEK3 1-15del cr748 evopreQ1
(SEQ ID NO: 257)
GGCCCAGACTGAGCACGTGAGTTTAAGAGCTAAGCTGGAAACAGCATAGC
AAGTTTAAATAAGGCTAGTCCGTTATCAACTCGAGAGAGTGGCACCGAGT
CGGTGCTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del cr289 evopreQ1
(SEQ ID NO: 258)
GGCCCAGACTGAGCACGTGAGTTTAAGAGCTAAGCTGGAAACAGCATAGC
AAGTTTAAATAAGGCTAGTCCGTTATCAACTGGAAACAGTGGCACCGAGT
CGGTGCTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del cr622 evopreQ1
(SEQ ID NO: 259)
GGCCCAGACTGAGCACGTGAGTTTAAGAGCTAAGCTGGAAACAGCATAGC
AAGTTTAAATAAGGCTAGTCCGTTATCAGCTGGAAACAGCGGCACCGAGT
CGGTGCTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del cr772 evopreQ1
(SEQ ID NO: 260)
GGCCCAGACTGAGCACGTGAGTTTAAGAGCTAAGCTGGAAACAGCATAGC
AAGTTTAAATAAGGCTAGTCCGTTATCAACTCGAAAGAGTGGCACCGAGT
CGGTGCTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del cr532 evopreQ1
(SEQ ID NO: 261)
GGCCCAGACTGAGCACGTGAGTTTAAGAGCTAAGCTGGAAACAGCATAGC
AAGTTTAAATAAGGCTAGTCCGTTATCAACGTGAAAACGTGACTCCGAGT
CGGAGTTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del cr961 evopreQ1
(SEQ ID NO: 262)
GGCCCAGACTGAGCACGTGAGTTTAAGAGCTAAGCTGGAAACAGCATAGC
AAGTTTAAATAAGGCTAGTCCGTTATCAACTCGAAAGAGTGCAACCGAGT
CGGTTGTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
HEK3 1-15del flip and extension
scaffold evopreQ1
(SEQ ID NO: 263)
GGCCCAGACTGAGCACGTGAGTTTAAGAGCTAAGCTGGAAACAGCATAGC
AAGTTTAAATAAGGCTAGTCCGTTATCAACTTGAAAAAGTGGCACCGAGT
CGGTGCTGCCCTCTGGAGGAAGCAGGGCTTCCCGTGCTCAGTCTGTCTCT
CTCTTGACGCGGTTCTATCTAGTTACGCGTTAAACCAACTAGAAATTTTT
T
RNF2 1-15del cr748 evopreQ1
(SEQ ID NO: 264)
GTCATCTTAGTCATTACCTGGTTTAAGAGCTAAGCTGGAAACAGCATAGC
AAGTTTAAATAAGGCTAGTCCGTTATCAACTCGAGAGAGTGGCACCGAGT
CGGTGCTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del cr289 evopreQ1
(SEQ ID NO: 265)
GTCATCTTAGTCATTACCTGGTTTAAGAGCTAAGCTGGAAACAGCATAGC
AAGTTTAAATAAGGCTAGTCCGTTATCAACTGGAAACAGTGGCACCGAGT
CGGTGCTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del cr622 evopreQ1
(SEQ ID NO: 266)
GTCATCTTAGTCATTACCTGGTTTAAGAGCTAAGCTGGAAACAGCATAGC
AAGTTTAAATAAGGCTAGTCCGTTATCAGCTGGAAACAGCGGCACCGAGT
CGGTGCTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del cr772 evopreQ1
(SEQ ID NO: 267)
GTCATCTTAGTCATTACCTGGTTTAAGAGCTAAGCTGGAAACAGCATAGC
AAGTTTAAATAAGGCTAGTCCGTTATCAACTCGAAAGAGTGGCACCGAGT
CGGTGCTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del cr532 evopreQ1
(SEQ ID NO: 268)
GTCATCTTAGTCATTACCTGGTTTAAGAGCTAAGCTGGAAACAGCATAGC
AAGTTTAAATAAGGCTAGTCCGTTATCAACGTGAAAACGTGACTCCGAGT
CGGAGTTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del cr961 evopreQ1
(SEQ ID NO: 269)
GTCATCTTAGTCATTACCTGGTTTAAGAGCTAAGCTGGAAACAGCATAGC
AAGTTTAAATAAGGCTAGTCCGTTATCAACTCGAAAGAGTGCAACCGAGT
CGGTTGTATGGGAACTCAGTTTATATGAGTTAGTAATGACTAAGATGTCA
TCTCTTTGACGCGGTTCTATCTAGTTACGCGTTAAACCAACTAGAAATTT
TTT
RNF2 1-15del flip and extension
scaffold evopreQ1
(SEQ ID NO: 270)
GTCATCTTAGTCATTACCTGGTTTAAGAGCTAAGCTGGAAACAGCATAGC
AAGTTTAAATAAGGCTAGTCCGTTATCAACTTGAAAAAGTGGCACCGAGT
CGGTGCTATGGGAACTCAGTTTATATGAGTTAGTAATGACTAAGATGTCA
TCTCTTTGACGCGGTTCTATCTAGTTACGCGTTAAACCAACTAGAAATTT
TTT
RUNX1 1-15del standard scaffold
evopreQ1
(SEQ ID NO: 271)
GCATTTTCAGGAGGAAGCGAGTTTTAGAGCTAGAAATAGCAAGTTAAAAT
AAGGCTAGTCCGTTATCAACTTGAAAAAGTGGCACCGAGTCGGTGCTACG
AAGGAAATGACTCAAATATGCCTTCCTCCTGAAAATAACTCTCTTTGACG
CGGTTCTATCTAGTTACGCGTTAAACCAACTAGAAATTTTTT
RUNX1 1-15del cr748 evopreQ1
(SEQ ID NO: 272)
GCATTTTCAGGAGGAAGCGAGTTTAAGAGCTAAGCTGGAAACAGCATAGC
AAGTTTAAATAAGGCTAGTCCGTTATCAACTCGAGAGAGTGGCACCGAGT
CGGTGCTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del cr289 evopreQ1
(SEQ ID NO: 273)
GCATTTTCAGGAGGAAGCGAGTTTAAGAGCTAAGCTGGAAACAGCATAGC
AAGTTTAAATAAGGCTAGTCCGTTATCAACTGGAAACAGTGGCACCGAGT
CGGTGCTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del cr622 evopreQ1
(SEQ ID NO: 274)
GCATTTTCAGGAGGAAGCGAGTTTAAGAGCTAAGCTGGAAACAGCATAGC
AAGTTTAAATAAGGCTAGTCCGTTATCAGCTGGAAACAGCGGCACCGAGT
CGGTGCTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del cr772 evopreQ1
(SEQ ID NO: 275)
GCATTTTCAGGAGGAAGCGAGTTTAAGAGCTAAGCTGGAAACAGCATAGC
AAGTTTAAATAAGGCTAGTCCGTTATCAACTCGAAAGAGTGGCACCGAGT
CGGTGCTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del cr532 evopreQ1
(SEQ ID NO: 276)
GCATTTTCAGGAGGAAGCGAGTTTAAGAGCTAAGCTGGAAACAGCATAGC
AAGTTTAAATAAGGCTAGTCCGTTATCAACGTGAAAACGTGACTCCGAGT
CGGAGTTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del cr961 evopreQ1
(SEQ ID NO: 277)
GCATTTTCAGGAGGAAGCGAGTTTAAGAGCTAAGCTGGAAACAGCATAGC
AAGTTTAAATAAGGCTAGTCCGTTATCAACTCGAAAGAGTGCAACCGAGT
CGGTTGTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 1-15del flip and extension
scaffold evopreQ1
(SEQ ID NO: 278)
GCATTTTCAGGAGGAAGCGAGTTTAAGAGCTAAGCTGGAAACAGCATAGC
AAGTTTAAATAAGGCTAGTCCGTTATCAACTTGAAAAAGTGGCACCGAGT
CGGTGCTTACGAAGGAAATGACTCAAATATGCCTTCCTCCTGAAAATAAC
TCTCTTTGACGCGGTTCTATCTAGTTACGCGTTAAACCAACTAGAAATTT
TTT
RUNX1 +5G-T standard scaffold
evopreQ1
(SEQ ID NO: 279)
GCATTTTCAGGAGGAAGCGAGTTTTAGAGCTAGAAATAGCAAGTTAAAAT
AAGGCTAGTCCGTTATCAACTTGAAAAAGTGGCACCGAGTCGGTGCTGTC
TGAAGCAATCGCTTCCTCCTGAAAATAACTCTCTTTGACGCGGTTCTATC
TAGTTACGCGTTAAACCAACTAGAAATTTTTT
RUNX1 +5G-T cr748 evopreQ1
(SEQ ID NO: 280)
GCATTTTCAGGAGGAAGCGAGTTTAAGAGCTAAGCTGGAAACAGCATAGC
AAGTTTAAATAAGGCTAGTCCGTTATCAACTCGAGAGAGTGGCACCGAGT
CGGTGCTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T cr289 evopreQ1
(SEQ ID NO: 281)
GCATTTTCAGGAGGAAGCGAGTTTAAGAGCTAAGCTGGAAACAGCATAGC
AAGTTTAAATAAGGCTAGTCCGTTATCAACTGGAAACAGTGGCACCGAGT
CGGTGCTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T cr622 evopreQ1
(SEQ ID NO: 282)
GCATTTTCAGGAGGAAGCGAGTTTAAGAGCTAAGCTGGAAACAGCATAGC
AAGTTTAAATAAGGCTAGTCCGTTATCAGCTGGAAACAGCGGCACCGAGT
CGGTGCTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T cr772 evopreQ1
(SEQ ID NO: 283)
GCATTTTCAGGAGGAAGCGAGTTTAAGAGCTAAGCTGGAAACAGCATAGC
AAGTTTAAATAAGGCTAGTCCGTTATCAACTCGAAAGAGTGGCACCGAGT
CGGTGCTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T cr532 evopreQ1
(SEQ ID NO: 284)
GCATTTTCAGGAGGAAGCGAGTTTAAGAGCTAAGCTGGAAACAGCATAGC
AAGTTTAAATAAGGCTAGTCCGTTATCAACGTGAAAACGTGACTCCGAGT
CGGAGTTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T cr961 evopreQ1
(SEQ ID NO: 285)
GCATTTTCAGGAGGAAGCGAGTTTAAGAGCTAAGCTGGAAACAGCATAGC
AAGTTTAAATAAGGCTAGTCCGTTATCAACTCGAAAGAGTGCAACCGAGT
CGGTTGTTGTCTGAAGCAATCGCTTCCTCCTGAAAATAACTCTCTTTGAC
GCGGTTCTATCTAGTTACGCGTTAAACCAACTAGAAATTTTTT
RUNX1 +5G-T flip and extension
scaffold evopreQ1
(SEQ ID NO: 286)
GCATTTTCAGGAGGAAGCGAGTTTAAGAGCTAAGCTGGAAACAGCATAGC
AAGTTTAAATAAGGCTAGTCCGTTATCAACTTGAAAAAGTGGCACCGAGT
CGGTGCTTGTCTGAAGCAATCGCTTCCTCCTGAAAATAACTCTCTTTGAC
GCGGTTCTATCTAGTTACGCGTTAAACCAACTAGAAATTTTTT
DNMT1 1-15del standard scaffold
evopreQ1
(SEQ ID NO: 287)
GATTCCTGGTGCCAGAAACAGTTTTAGAGCTAGAAATAGCAAGTTAAAAT
AAGGCTAGTCCGTTATCAACTTGAAAAAGTGGCACCGAGTCGGTGCTGCT
AAGGACTAGTTCTGCCCTTCTGGCACCAGGACCTCTTCTTTGACGCGGTT
CTATCTAGTTACGCGTTAAACCAACTAGAAATTTTTT
DNMT1 1-15del cr748 evopreQ1
(SEQ ID NO: 288)
GATTCCTGGTGCCAGAAACAGTTTAAGAGCTAAGCTGGAAACAGCATAGC
AAGTTTAAATAAGGCTAGTCCGTTATCAACTCGAGAGAGTGGCACCGAGT
CGGTGCTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del cr289 evopreQ1
(SEQ ID NO: 289)
GATTCCTGGTGCCAGAAACAGTTTAAGAGCTAAGCTGGAAACAGCATAGC
AAGTTTAAATAAGGCTAGTCCGTTATCAACTGGAAACAGTGGCACCGAGT
CGGTGCTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del cr622 evopreQ1
(SEQ ID NO: 290)
GATTCCTGGTGCCAGAAACAGTTTAAGAGCTAAGCTGGAAACAGCATAGC
AAGTTTAAATAAGGCTAGTCCGTTATCAGCTGGAAACAGCGGCACCGAGT
CGGTGCTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del cr772 evopreQ1
(SEQ ID NO: 291)
GATTCCTGGTGCCAGAAACAGTTTAAGAGCTAAGCTGGAAACAGCATAGC
AAGTTTAAATAAGGCTAGTCCGTTATCAACTCGAAAGAGTGGCACCGAGT
CGGTGCTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del cr532 evopreQ1
(SEQ ID NO: 292)
GATTCCTGGTGCCAGAAACAGTTTAAGAGCTAAGCTGGAAACAGCATAGC
AAGTTTAAATAAGGCTAGTCCGTTATCAACGTGAAAACGTGACTCCGAGT
CGGAGTTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del cr961 evopreQ1
(SEQ ID NO: 293)
GATTCCTGGTGCCAGAAACAGTTTAAGAGCTAAGCTGGAAACAGCATAGC
AAGTTTAAATAAGGCTAGTCCGTTATCAACTCGAAAGAGTGCAACCGAGT
CGGTTGTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 1-15del flip and extension
scaffold evopreQ1
(SEQ ID NO: 294)
GATTCCTGGTGCCAGAAACAGTTTAAGAGCTAAGCTGGAAACAGCATAGC
AAGTTTAAATAAGGCTAGTCCGTTATCAACTTGAAAAAGTGGCACCGAGT
CGGTGCTTGCTAAGGACTAGTTCTGCCCTTCTGGCACCAGGACCTCTTCT
TTGACGCGGTTCTATCTAGTTACGCGTTAAACCAACTAGAAATTTTTT
DNMT1 +5 G--T standard scaffold evopreQ1
(SEQ ID NO: 295)
GATTCCTGGTGCCAGAAACAGTTTTAGAGCTAGAAATAGCAAGTTAAAAT
AAGGCTAGTCCGTTATCAACTTGAAAAAGTGGCACCGAGTCGGTGCGTCA
CCACTGTTTCTGGCACCAGGACCTCTTCTTTGACGCGGTTCTATCTAGTT
ACGCGTTAAACCAACTAGAAATTTTTT
DNMT1 +5 G--T cr748 evopreQ1
(SEQ ID NO: 296)
GATTCCTGGTGCCAGAAACAGTTTAAGAGCTAAGCTGGAAACAGCATAGC
AAGTTTAAATAAGGCTAGTCCGTTATCAACTCGAGAGAGTGGCACCGAGT
CGGTGCTGTCACCACTGTTTCTGGCACCAGGACCTCTTCTTTGACGCGGT
TCTATCTAGTTACGCGTTAAACCAACTAGAAATTTTTT
DNMT1 +5 G--T cr289 evopreQ1
(SEQ ID NO: 297)
GATTCCTGGTGCCAGAAACAGTTTAAGAGCTAAGCTGGAAACAGCATAGC
AAGTTTAAATAAGGCTAGTCCGTTATCAACTGGAAACAGTGGCACCGAGT
CGGTGCTGTCACCACTGTTTCTGGCACCAGGACCTCTTCTTTGACGCGGT
TCTATCTAGTTACGCGTTAAACCAACTAGAAATTTTTT
DNMT1 +5 G--T cr622 evopreQ1
(SEQ ID NO: 298)
GATTCCTGGTGCCAGAAACAGTTTAAGAGCTAAGCTGGAAACAGCATAGC
AAGTTTAAATAAGGCTAGTCCGTTATCAGCTGGAAACAGCGGCACCGAGT
CGGTGCTGTCACCACTGTTTCTGGCACCAGGACCTCTTCTTTGACGCGGT
TCTATCTAGTTACGCGTTAAACCAACTAGAAATTTTTT
DNMT1 +5 G--T cr772 evopreQ1
(SEQ ID NO: 299)
GATTCCTGGTGCCAGAAACAGTTTAAGAGCTAAGCTGGAAACAGCATAGC
AAGTTTAAATAAGGCTAGTCCGTTATCAACTCGAAAGAGTGGCACCGAGT
CGGTGCTGTCACCACTGTTTCTGGCACCAGGACCTCTTCTTTGACGCGGT
TCTATCTAGTTACGCGTTAAACCAACTAGAAATTTTTT
DNMT1 +5 G--T cr532 evopreQ1
(SEQ ID NO: 300)
GATTCCTGGTGCCAGAAACAGTTTAAGAGCTAAGCTGGAAACAGCATAGC
AAGTTTAAATAAGGCTAGTCCGTTATCAACGTGAAAACGTGACTCCGAGT
CGGAGTTGTCACCACTGTTTCTGGCACCAGGACCTCTTCTTTGACGCGGT
TCTATCTAGTTACGCGTTAAACCAACTAGAAATTTTTT
DNMT1 +5 G--T cr961 evopreQ1
(SEQ ID NO: 301)
GATTCCTGGTGCCAGAAACAGTTTAAGAGCTAAGCTGGAAACAGCATAGC
AAGTTTAAATAAGGCTAGTCCGTTATCAACTCGAAAGAGTGCAACCGAGT
CGGTTGTGTCACCACTGTTTCTGGCACCAGGACCTCTTCTTTGACGCGGT
TCTATCTAGTTACGCGTTAAACCAACTAGAAATTTTTT
DNMT1 +5 G--T flip and extension
scaffold evopreQ1
(SEQ ID NO: 302)
GATTCCTGGTGCCAGAAACAGTTTAAGAGCTAAGCTGGAAACAGCATAGC
AAGTTTAAATAAGGCTAGTCCGTTATCAACTTGAAAAAGTGGCACCGAGT
CGGTGCTGTCACCACTGTTTCTGGCACCAGGACCTCTTCTTTGACGCGGT
TCTATCTAGTTACGCGTTAAACCAACTAGAAATTTTTT
FANCF 1-15del standard scaffold
evopreQ1
(SEQ ID NO: 303)
GGAATCCCTTCTGCAGCACCGTTTTAGAGCTAGAAATAGCAAGTTAAAAT
AAGGCTAGTCCGTTATCAACTTGAAAAAGTGGCACCGAGTCGGTGCTAGT
GCTTGAGACCGCCAGAAGCTCGGGCTGCAGAAGGGACAATCACTTTGACG
CGGTTCTATCTAGTTACGCGTTAAACCAACTAGAAATTTTTT
FANCF 1-15del cr748 evopreQ1
(SEQ ID NO: 304)
GGAATCCCTTCTGCAGCACCGTTTAAGAGCTAAGCTGGAAACAGCATAGC
AAGTTTAAATAAGGCTAGTCCGTTATCAACTCGAGAGAGTGGCACCGAGT
CGGTGCTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del cr289 evopreQ1
(SEQ ID NO: 305)
GGAATCCCTTCTGCAGCACCGTTTAAGAGCTAAGCTGGAAACAGCATAGC
AAGTTTAAATAAGGCTAGTCCGTTATCAACTGGAAACAGTGGCACCGAGT
CGGTGCTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del cr622 evopreQ1
(SEQ ID NO: 306)
GGAATCCCTTCTGCAGCACCGTTTAAGAGCTAAGCTGGAAACAGCATAGC
AAGTTTAAATAAGGCTAGTCCGTTATCAGCTGGAAACAGCGGCACCGAGT
CGGTGCTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del cr772 evopreQ1
(SEQ ID NO: 307)
GGAATCCCTTCTGCAGCACCGTTTAAGAGCTAAGCTGGAAACAGCATAGC
AAGTTTAAATAAGGCTAGTCCGTTATCAACTCGAAAGAGTGGCACCGAGT
CGGTGCTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del cr532 evopreQ1
(SEQ ID NO: 308)
GGAATCCCTTCTGCAGCACCGTTTAAGAGCTAAGCTGGAAACAGCATAGC
AAGTTTAAATAAGGCTAGTCCGTTATCAACGTGAAAACGTGACTCCGAGT
CGGAGTTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del cr961 evopreQ1
(SEQ ID NO: 309)
GGAATCCCTTCTGCAGCACCGTTTAAGAGCTAAGCTGGAAACAGCATAGC
AAGTTTAAATAAGGCTAGTCCGTTATCAACTCGAAAGAGTGCAACCGAGT
CGGTTGTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF 1-15del flip and extension
scaffold evopreQ1
(SEQ ID NO: 310)
GGAATCCCTTCTGCAGCACCGTTTAAGAGCTAAGCTGGAAACAGCATAGC
AAGTTTAAATAAGGCTAGTCCGTTATCAACTTGAAAAAGTGGCACCGAGT
CGGTGCTTAGTGCTTGAGACCGCCAGAAGCTCGGGCTGCAGAAGGGACAA
TCACTTTGACGCGGTTCTATCTAGTTACGCGTTAAACCAACTAGAAATTT
TTT
FANCF +5 G--T cr748 evopreQ1
(SEQ ID NO: 311)
GGAATCCCTTCTGCAGCACCGTTTAAGAGCTAAGCTGGAAACAGCATAGC
AAGTTTAAATAAGGCTAGTCCGTTATCAACTCGAGAGAGTGGCACCGAGT
CGGTGCTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T cr289 evopreQ1
(SEQ ID NO: 312)
GGAATCCCTTCTGCAGCACCGTTTAAGAGCTAAGCTGGAAACAGCATAGC
AAGTTTAAATAAGGCTAGTCCGTTATCAACTGGAAACAGTGGCACCGAGT
CGGTGCTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T cr622 evopreQ1
(SEQ ID NO: 313)
GGAATCCCTTCTGCAGCACCGTTTAAGAGCTAAGCTGGAAACAGCATAGC
AAGTTTAAATAAGGCTAGTCCGTTATCAGCTGGAAACAGCGGCACCGAGT
CGGTGCTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T cr772 evopreQ1
(SEQ ID NO: 314)
GGAATCCCTTCTGCAGCACCGTTTAAGAGCTAAGCTGGAAACAGCATAGC
AAGTTTAAATAAGGCTAGTCCGTTATCAACTCGAAAGAGTGGCACCGAGT
CGGTGCTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T cr532 evopreQ1
(SEQ ID NO: 315)
GGAATCCCTTCTGCAGCACCGTTTAAGAGCTAAGCTGGAAACAGCATAGC
AAGTTTAAATAAGGCTAGTCCGTTATCAACGTGAAAACGTGACTCCGAGT
CGGAGTTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T cr961 evopreQ1
(SEQ ID NO: 316)
GGAATCCCTTCTGCAGCACCGTTTAAGAGCTAAGCTGGAAACAGCATAGC
AAGTTTAAATAAGGCTAGTCCGTTATCAACTCGAAAGAGTGCAACCGAGT
CGGTTGTGGAAAAGCGATCAAGGTGCTGCAGAAGGGACAATCACTTTGAC
GCGGTTCTATCTAGTTACGCGTTAAACCAACTAGAAATTTTTT
FANCF +5 G--T flip and extension
scaffold evopreQ1
(SEQ ID NO: 317)
GGAATCCCTTCTGCAGCACCGTTTAAGAGCTAAGCTGGAAACAGCATAGC
AAGTTTAAATAAGGCTAGTCCGTTATCAACTTGAAAAAGTGGCACCGAGT
CGGTGCTGGAAAAGCGATCAAGGTGCTGCAGAAGGGACAATCACTTTGAC
GCGGTTCTATCTAGTTACGCGTTAAACCAACTAGAAATTTTTT
EMX1 1-15del standard scaffold
evopreQ1
(SEQ ID NO: 318)
GAGTCCGAGCAGAAGAAGAAGTTTTAGAGCTAGAAATAGCAAGTTAAAAT
AAGGCTAGTCCGTTATCAACTTGAAAAAGTGGCACCGAGTCGGTGCTCGT
GGCAATGCGCCACCGGTTGATGTTCTTCTGCTCGGAAACAATCTTTGACG
CGGTTCTATCTAGTTACGCGTTAAACCAACTAGAAATTTTTT
EMX1 1-15del cr748 evopreQ1
(SEQ ID NO: 319)
GAGTCCGAGCAGAAGAAGAAGTTTAAGAGCTAAGCTGGAAACAGCATAGC
AAGTTTAAATAAGGCTAGTCCGTTATCAACTCGAGAGAGTGGCACCGAGT
CGGTGCTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del cr289 evopreQ1
(SEQ ID NO: 320)
GAGTCCGAGCAGAAGAAGAAGTTTAAGAGCTAAGCTGGAAACAGCATAGC
AAGTTTAAATAAGGCTAGTCCGTTATCAACTGGAAACAGTGGCACCGAGT
CGGTGCTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del cr622 evopreQ1
(SEQ ID NO: 321)
GAGTCCGAGCAGAAGAAGAAGTTTAAGAGCTAAGCTGGAAACAGCATAGC
AAGTTTAAATAAGGCTAGTCCGTTATCAGCTGGAAACAGCGGCACCGAGT
CGGTGCTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del cr772 evopreQ1
(SEQ ID NO: 322)
GAGTCCGAGCAGAAGAAGAAGTTTAAGAGCTAAGCTGGAAACAGCATAGC
AAGTTTAAATAAGGCTAGTCCGTTATCAACTCGAAAGAGTGGCACCGAGT
CGGTGCTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del cr532 evopreQ1
(SEQ ID NO: 323)
GAGTCCGAGCAGAAGAAGAAGTTTAAGAGCTAAGCTGGAAACAGCATAGC
AAGTTTAAATAAGGCTAGTCCGTTATCAACGTGAAAACGTGACTCCGAGT
CGGAGTTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del cr961 evopreQ1
(SEQ ID NO: 324)
GAGTCCGAGCAGAAGAAGAAGTTTAAGAGCTAAGCTGGAAACAGCATAGC
AAGTTTAAATAAGGCTAGTCCGTTATCAACTCGAAAGAGTGCAACCGAGT
CGGTTGTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 1-15del flip and extension
scaffold evopreQ1
(SEQ ID NO: 325)
GAGTCCGAGCAGAAGAAGAAGTTTAAGAGCTAAGCTGGAAACAGCATAGC
AAGTTTAAATAAGGCTAGTCCGTTATCAACTTGAAAAAGTGGCACCGAGT
CGGTGCTTCGTGGCAATGCGCCACCGGTTGATGTTCTTCTGCTCGGAAAC
AATCTTTGACGCGGTTCTATCTAGTTACGCGTTAAACCAACTAGAAATTT
TTT
EMX1 +5 G--T standard scaffold
evopreQ1
(SEQ ID NO: 326)
GAGTCCGAGCAGAAGAAGAAGTTTTAGAGCTAGAAATAGCAAGTTAAAAT
AAGGCTAGTCCGTTATCAACTTGAAAAAGTGGCACCGAGTCGGTGCGTGA
TGGGAGCACTTCTTCTTCTGCTCGGAAACAATCTTTGACGCGGTTCTATC
TAGTTACGCGTTAAACCAACTAGAAATTTTTT
EMX1 +5 G--T cr748 evopreQ1
(SEQ ID NO: 327)
GAGTCCGAGCAGAAGAAGAAGTTTAAGAGCTAAGCTGGAAACAGCATAGC
AAGTTTAAATAAGGCTAGTCCGTTATCAACTCGAGAGAGTGGCACCGAGT
CGGTGCTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T cr289 evopreQ1
(SEQ ID NO: 328)
GAGTCCGAGCAGAAGAAGAAGTTTAAGAGCTAAGCTGGAAACAGCATAGC
AAGTTTAAATAAGGCTAGTCCGTTATCAACTGGAAACAGTGGCACCGAGT
CGGTGCTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T cr622 evopreQ1
(SEQ ID NO: 329)
GAGTCCGAGCAGAAGAAGAAGTTTAAGAGCTAAGCTGGAAACAGCATAGC
AAGTTTAAATAAGGCTAGTCCGTTATCAGCTGGAAACAGCGGCACCGAGT
CGGTGCTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T cr772 evopreQ1
(SEQ ID NO: 330)
GAGTCCGAGCAGAAGAAGAAGTTTAAGAGCTAAGCTGGAAACAGCATAGC
AAGTTTAAATAAGGCTAGTCCGTTATCAACTCGAAAGAGTGGCACCGAGT
CGGTGCTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T cr532 evopreQ1
(SEQ ID NO: 331)
GAGTCCGAGCAGAAGAAGAAGTTTAAGAGCTAAGCTGGAAACAGCATAGC
AAGTTTAAATAAGGCTAGTCCGTTATCAACGTGAAAACGTGACTCCGAGT
CGGAGTTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T cr961 evopreQ1
(SEQ ID NO: 332)
GAGTCCGAGCAGAAGAAGAAGTTTAAGAGCTAAGCTGGAAACAGCATAGC
AAGTTTAAATAAGGCTAGTCCGTTATCAACTCGAAAGAGTGCAACCGAGT
CGGTTGTGTGATGGGAGCACTTCTTCTTCTGCTCGGAAACAATCTTTGAC
GCGGTTCTATCTAGTTACGCGTTAAACCAACTAGAAATTTTTT
EMX1 +5 G--T flip and extension
scaffold evopreQ1
(SEQ ID NO: 333)
GAGTCCGAGCAGAAGAAGAAGTTTAAGAGCTAAGCTGGAAACAGCATAGC
AAGTTTAAATAAGGCTAGTCCGTTATCAACTTGAAAAAGTGGCACCGAGT
CGGTGCTGTGATGGGAGCACTTCTTCTTCTGCTCGGAAACAATCTTTGAC
GCGGTTCTATCTAGTTACGCGTTAAACCAACTAGAAATTTTTT
RNF2 +1FLAG standard scaffold
evopreQ1
(SEQ ID NO: 334)
GTCATCTTAGTCATTACCTGGTTTTAGAGCTAGAAATAGCAAGTTAAAAT
AAGGCTAGTCCGTTATCAACTTGAAAAAGTGGCACCGAGTCGGTGCTGAG
TTACAACGAACACCTCAGCTTATCGTCGTCATCCTTGTAATCGTAATGAC
TAAGATGTCATCTCTTTGACGCGGTTCTATCTAGTTACGCGTTAAACCAA
CTAGAAATTTTTT
RNF2 +1FLAG cr748 evopreQ1
(SEQ ID NO: 335)
GTCATCTTAGTCATTACCTGGTTTAAGAGCTAAGCTGGAAACAGCATAGC
AAGTTTAAATAAGGCTAGTCCGTTATCAACTCGAGAGAGTGGCACCGAGT
CGGTGCTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG cr289 evopreQ1
(SEQ ID NO: 336)
GTCATCTTAGTCATTACCTGGTTTAAGAGCTAAGCTGGAAACAGCATAGC
AAGTTTAAATAAGGCTAGTCCGTTATCAACTGGAAACAGTGGCACCGAGT
CGGTGCTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG cr622 evopreQ1
(SEQ ID NO: 337)
GTCATCTTAGTCATTACCTGGTTTAAGAGCTAAGCTGGAAACAGCATAGC
AAGTTTAAATAAGGCTAGTCCGTTATCAGCTGGAAACAGCGGCACCGAGT
CGGTGCTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG cr772 evopreQ1
(SEQ ID NO: 338)
GTCATCTTAGTCATTACCTGGTTTAAGAGCTAAGCTGGAAACAGCATAGC
AAGTTTAAATAAGGCTAGTCCGTTATCAACTCGAAAGAGTGGCACCGAGT
CGGTGCTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG cr532 evopreQ1
(SEQ ID NO: 339)
GTCATCTTAGTCATTACCTGGTTTAAGAGCTAAGCTGGAAACAGCATAGC
AAGTTTAAATAAGGCTAGTCCGTTATCAACGTGAAAACGTGACTCCGAGT
CGGAGTTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG cr961 evopreQ1
(SEQ ID NO: 340)
GTCATCTTAGTCATTACCTGGTTTAAGAGCTAAGCTGGAAACAGCATAGC
AAGTTTAAATAAGGCTAGTCCGTTATCAACTCGAAAGAGTGCAACCGAGT
CGGTTGTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
RNF2 +1FLAG flip and extension
scaffold evopreQ1
(SEQ ID NO: 341)
GTCATCTTAGTCATTACCTGGTTTAAGAGCTAAGCTGGAAACAGCATAGC
AAGTTTAAATAAGGCTAGTCCGTTATCAACTTGAAAAAGTGGCACCGAGT
CGGTGCTTGAGTTACAACGAACACCTCAGCTTATCGTCGTCATCCTTGTA
ATCGTAATGACTAAGATGTCATCTCTTTGACGCGGTTCTATCTAGTTACG
CGTTAAACCAACTAGAAATTTTTT
VEGFA +5 G--T cr748 evopreQ1
(SEQ ID NO: 342)
GATGTCTGCAGGCCAGATGAGTTTAAGAGCTAAGCTGGAAACAGCATAGC
AAGTTTAAATAAGGCTAGTCCGTTATCAACTCGAGAGAGTGGCACCGAGT
CGGTGCTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T cr289 evopreQ1
(SEQ ID NO: 343)
GATGTCTGCAGGCCAGATGAGTTTAAGAGCTAAGCTGGAAACAGCATAGC
AAGTTTAAATAAGGCTAGTCCGTTATCAACTGGAAACAGTGGCACCGAGT
CGGTGCTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T cr622 evopreQ1
(SEQ ID NO: 344)
GATGTCTGCAGGCCAGATGAGTTTAAGAGCTAAGCTGGAAACAGCATAGC
AAGTTTAAATAAGGCTAGTCCGTTATCAGCTGGAAACAGCGGCACCGAGT
CGGTGCTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T cr772 evopreQ1
(SEQ ID NO: 345)
GATGTCTGCAGGCCAGATGAGTTTAAGAGCTAAGCTGGAAACAGCATAGC
AAGTTTAAATAAGGCTAGTCCGTTATCAACTCGAAAGAGTGGCACCGAGT
CGGTGCTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T cr532 evopreQ1
(SEQ ID NO: 346)
GATGTCTGCAGGCCAGATGAGTTTAAGAGCTAAGCTGGAAACAGCATAGC
AAGTTTAAATAAGGCTAGTCCGTTATCAACGTGAAAACGTGACTCCGAGT
CGGAGTTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T cr961 evopreQ1
(SEQ ID NO: 347)
GATGTCTGCAGGCCAGATGAGTTTAAGAGCTAAGCTGGAAACAGCATAGC
AAGTTTAAATAAGGCTAGTCCGTTATCAACTCGAAAGAGTGCAACCGAGT
CGGTTGTAATGTGCCATCTGGAGCACTCATCTGGCCTGCAGAACAATCTC
TTGACGCGGTTCTATCTAGTTACGCGTTAAACCAACTAGAAATTTTTT
VEGFA +5 G--T flip and extension
scaffold evopreQ1
(SEQ ID NO: 348)
GATGTCTGCAGGCCAGATGAGTTTAAGAGCTAAGCTGGAAACAGCATAGC
AAGTTTAAATAAGGCTAGTCCGTTATCAACTTGAAAAAGTGGCACCGAGT
CGGTGCTAATGTGCCATCTGGAGCACTCATCTGGCCTGCAGAACAATCTC
TTGACGCGGTTCTATCTAGTTACGCGTTAAACCAACTAGAAATTTTTT
VEGFA +1FLAG standard scaffold
evopreQ1
(SEQ ID NO: 349)
GATGTCTGCAGGCCAGATGAGTTTTAGAGCTAGAAATAGCAAGTTAAAAT
AAGGCTAGTCCGTTATCAACTTGAAAAAGTGGCACCGAGTCGGTGCAATG
TGCCATCTGGAGCACTCACTTATCGTCGTCATCCTTGTAATCTCTGGCCT
GCAGAACAATCTCTTGACGCGGTTCTATCTAGTTACGCGTTAAACCAACT
AGAAATTTTTT
VEGFA +1FLAG cr748 evopreQ1
(SEQ ID NO: 350)
GATGTCTGCAGGCCAGATGAGTTTAAGAGCTAAGCTGGAAACAGCATAGC
AAGTTTAAATAAGGCTAGTCCGTTATCAACTCGAGAGAGTGGCACCGAGT
CGGTGCTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG cr289 evopreQ1
(SEQ ID NO: 351)
GATGTCTGCAGGCCAGATGAGTTTAAGAGCTAAGCTGGAAACAGCATAGC
AAGTTTAAATAAGGCTAGTCCGTTATCAACTGGAAACAGTGGCACCGAGT
CGGTGCTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG cr622 evopreQ1
(SEQ ID NO: 352)
GATGTCTGCAGGCCAGATGAGTTTAAGAGCTAAGCTGGAAACAGCATAGC
AAGTTTAAATAAGGCTAGTCCGTTATCAGCTGGAAACAGCGGCACCGAGT
CGGTGCTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG cr772 evopreQ1
(SEQ ID NO: 353)
GATGTCTGCAGGCCAGATGAGTTTAAGAGCTAAGCTGGAAACAGCATAGC
AAGTTTAAATAAGGCTAGTCCGTTATCAACTCGAAAGAGTGGCACCGAGT
CGGTGCTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG cr532 evopreQ1
(SEQ ID NO: 354)
GATGTCTGCAGGCCAGATGAGTTTAAGAGCTAAGCTGGAAACAGCATAGC
AAGTTTAAATAAGGCTAGTCCGTTATCAACGTGAAAACGTGACTCCGAGT
CGGAGTTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG cr961 evopreQ1
(SEQ ID NO: 355)
GATGTCTGCAGGCCAGATGAGTTTAAGAGCTAAGCTGGAAACAGCATAGC
AAGTTTAAATAAGGCTAGTCCGTTATCAACTCGAAAGAGTGCAACCGAGT
CGGTTGTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA +1FLAG flip and extension
scaffold evopreQ1
(SEQ ID NO: 356)
GATGTCTGCAGGCCAGATGAGTTTAAGAGCTAAGCTGGAAACAGCATAGC
AAGTTTAAATAAGGCTAGTCCGTTATCAACTTGAAAAAGTGGCACCGAGT
CGGTGCTAATGTGCCATCTGGAGCACTCACTTATCGTCGTCATCCTTGTA
ATCTCTGGCCTGCAGAACAATCTCTTGACGCGGTTCTATCTAGTTACGCG
TTAAACCAACTAGAAATTTTTT
VEGFA 1-15 del standard scaffold
evopreQ1
(SEQ ID NO: 357)
GATGTCTGCAGGCCAGATGAGTTTTAGAGCTAGAAATAGCAAGTTAAAAT
AAGGCTAGTCCGTTATCAACTTGAAAAAGTGGCACCGAGTCGGTGCGTGT
GTCCCTCTGACAATGTGCTCTGGCCTGCAGAACAATCTCTTGACGCGGTT
CTATCTAGTTACGCGTTAAACCAACTAGAAATTTTTT
VEGFA 1-15 del cr748 evopreQ1
(SEQ ID NO: 358)
GATGTCTGCAGGCCAGATGAGTTTAAGAGCTAAGCTGGAAACAGCATAGC
AAGTTTAAATAAGGCTAGTCCGTTATCAACTCGAGAGAGTGGCACCGAGT
CGGTGCTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del cr289 evopreQ1
(SEQ ID NO: 359)
GATGTCTGCAGGCCAGATGAGTTTAAGAGCTAAGCTGGAAACAGCATAGC
AAGTTTAAATAAGGCTAGTCCGTTATCAACTGGAAACAGTGGCACCGAGT
CGGTGCTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del cr622 evopreQ1
(SEQ ID NO: 360)
GATGTCTGCAGGCCAGATGAGTTTAAGAGCTAAGCTGGAAACAGCATAGC
AAGTTTAAATAAGGCTAGTCCGTTATCAGCTGGAAACAGCGGCACCGAGT
CGGTGCTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del cr772 evopreQ1
(SEQ ID NO: 361)
GATGTCTGCAGGCCAGATGAGTTTAAGAGCTAAGCTGGAAACAGCATAGC
AAGTTTAAATAAGGCTAGTCCGTTATCAACTCGAAAGAGTGGCACCGAGT
CGGTGCTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del cr532 evopreQ1
(SEQ ID NO: 362)
GATGTCTGCAGGCCAGATGAGTTTAAGAGCTAAGCTGGAAACAGCATAGC
AAGTTTAAATAAGGCTAGTCCGTTATCAACGTGAAAACGTGACTCCGAGT
CGGAGTTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del cr961 evopreQ1
(SEQ ID NO: 363)
GATGTCTGCAGGCCAGATGAGTTTAAGAGCTAAGCTGGAAACAGCATAGC
AAGTTTAAATAAGGCTAGTCCGTTATCAACTCGAAAGAGTGCAACCGAGT
CGGTTGTGTGTGTCCCTCTGACAATGTGCTCTGGCCTGCAGAACAATCTC
TTGACGCGGTTCTATCTAGTTACGCGTTAAACCAACTAGAAATTTTTT
VEGFA 1-15 del flip and extension
scaffold evopreQ1
(SEQ ID NO: 364)
GATGTCTGCAGGCCAGATGAGTTTAAGAGCTAAGCTGGAAACAGCATAGC
AAGTTTAAATAAGGCTAGTCCGTTATCAACTTGAAAAAGTGGCACCGAGT
CGGTGCTGTGTGTCCCTCTGACAATGTGCTCTGGCCTGCAGAACAATCTC
TTGACGCGGTTCTATCTAGTTACGCGTTAAACCAACTAGAAATTTTTT
RUNX1 +1FLAG standard scaffold
evopreQ1
(SEQ ID NO: 365)
GCATTTTCAGGAGGAAGCGAGTTTTAGAGCTAGAAATAGCAAGTTAAAAT
AAGGCTAGTCCGTTATCAACTTGAAAAAGTGGCACCGAGTCGGTGCTGTC
TGAAGCCATCCCTTATCGTCGTCATCCTTGTAATCCTTCCTCCTGAAAAT
AACTCTCTTTGACGCGGTTCTATCTAGTTACGCGTTAAACCAACTAGAAA
TTTTTT
RUNX1 +1FLAG cr748 evopreQ1
(SEQ ID NO: 366)
GCATTTTCAGGAGGAAGCGAGTTTAAGAGCTAAGCTGGAAACAGCATAGC
AAGTTTAAATAAGGCTAGTCCGTTATCAACTCGAGAGAGTGGCACCGAGT
CGGTGCTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG cr289 evopreQ1
(SEQ ID NO: 367)
GCATTTTCAGGAGGAAGCGAGTTTAAGAGCTAAGCTGGAAACAGCATAGC
AAGTTTAAATAAGGCTAGTCCGTTATCAACTGGAAACAGTGGCACCGAGT
CGGTGCTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG cr622 evopreQ1
(SEQ ID NO: 368)
GCATTTTCAGGAGGAAGCGAGTTTAAGAGCTAAGCTGGAAACAGCATAGC
AAGTTTAAATAAGGCTAGTCCGTTATCAGCTGGAAACAGCGGCACCGAGT
CGGTGCTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG cr772 evopreQ1
(SEQ ID NO: 369)
GCATTTTCAGGAGGAAGCGAGTTTAAGAGCTAAGCTGGAAACAGCATAGC
AAGTTTAAATAAGGCTAGTCCGTTATCAACTCGAAAGAGTGGCACCGAGT
CGGTGCTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG cr532 evopreQ1
(SEQ ID NO: 370)
GCATTTTCAGGAGGAAGCGAGTTTAAGAGCTAAGCTGGAAACAGCATAGC
AAGTTTAAATAAGGCTAGTCCGTTATCAACGTGAAAACGTGACTCCGAGT
CGGAGTTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG cr961 evopreQ1
(SEQ ID NO: 371)
GCATTTTCAGGAGGAAGCGAGTTTAAGAGCTAAGCTGGAAACAGCATAGC
AAGTTTAAATAAGGCTAGTCCGTTATCAACTCGAAAGAGTGCAACCGAGT
CGGTTGTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
RUNX1 +1FLAG flip and extension
scaffold evopreQ1
(SEQ ID NO: 372)
GCATTTTCAGGAGGAAGCGAGTTTAAGAGCTAAGCTGGAAACAGCATAGC
AAGTTTAAATAAGGCTAGTCCGTTATCAACTTGAAAAAGTGGCACCGAGT
CGGTGCTTGTCTGAAGCCATCCCTTATCGTCGTCATCCTTGTAATCCTTC
CTCCTGAAAATAACTCTCTTTGACGCGGTTCTATCTAGTTACGCGTTAAA
CCAACTAGAAATTTTTT
DNMT1 +1FLAG standard scaffold
evopreQ1
(SEQ ID NO: 373)
GATTCCTGGTGCCAGAAACAGTTTTAGAGCTAGAAATAGCAAGTTAAAAT
AAGGCTAGTCCGTTATCAACTTGAAAAAGTGGCACCGAGTCGGTGCTCTG
CCCTCCCGTCACCCCTGTCTTATCGTCGTCATCCTTGTAATCTTCTGGCA
CCAGGACCTCTTCTTTGACGCGGTTCTATCTAGTTACGCGTTAAACCAAC
TAGAAATTTTTT
DNMT1 +1FLAG cr748 evopreQ1
(SEQ ID NO: 374)
GATTCCTGGTGCCAGAAACAGTTTAAGAGCTAAGCTGGAAACAGCATAGC
AAGTTTAAATAAGGCTAGTCCGTTATCAACTCGAGAGAGTGGCACCGAGT
CGGTGCTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG cr289 evopreQ1
(SEQ ID NO: 375)
GATTCCTGGTGCCAGAAACAGTTTAAGAGCTAAGCTGGAAACAGCATAGC
AAGTTTAAATAAGGCTAGTCCGTTATCAACTGGAAACAGTGGCACCGAGT
CGGTGCTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG cr622 evopreQ1
(SEQ ID NO: 376)
GATTCCTGGTGCCAGAAACAGTTTAAGAGCTAAGCTGGAAACAGCATAGC
AAGTTTAAATAAGGCTAGTCCGTTATCAGCTGGAAACAGCGGCACCGAGT
CGGTGCTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG cr772 evopreQ1
(SEQ ID NO: 377)
GATTCCTGGTGCCAGAAACAGTTTAAGAGCTAAGCTGGAAACAGCATAGC
AAGTTTAAATAAGGCTAGTCCGTTATCAACTCGAAAGAGTGGCACCGAGT
CGGTGCTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG cr532 evopreQ1
(SEQ ID NO: 378)
GATTCCTGGTGCCAGAAACAGTTTAAGAGCTAAGCTGGAAACAGCATAGC
AAGTTTAAATAAGGCTAGTCCGTTATCAACGTGAAAACGTGACTCCGAGT
CGGAGTTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG cr961 evopreQ1
(SEQ ID NO: 379)
GATTCCTGGTGCCAGAAACAGTTTAAGAGCTAAGCTGGAAACAGCATAGC
AAGTTTAAATAAGGCTAGTCCGTTATCAACTCGAAAGAGTGCAACCGAGT
CGGTTGTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT
DNMT1 +1FLAG flip and extension
scaffold evopreQ1
(SEQ ID NO: 380)
GATTCCTGGTGCCAGAAACAGTTTAAGAGCTAAGCTGGAAACAGCATAGC
AAGTTTAAATAAGGCTAGTCCGTTATCAACTTGAAAAAGTGGCACCGAGT
CGGTGCTTCTGCCCTCCCGTCACCCCTGTCTTATCGTCGTCATCCTTGTA
ATCTTCTGGCACCAGGACCTCTTCTTTGACGCGGTTCTATCTAGTTACGC
GTTAAACCAACTAGAAATTTTTT

The present disclosure contemplates any such ways to further improve the efficacy of the prime editing systems disclosed here.

In various embodiments, it may be advantageous to limit the appearance of consecutive sequence of Ts from the extension arm as consecutive series of T's may limit the capacity of the pegRNA to be transcribed. For example, strings of at least consecutive three T's, at least consecutive four T's, at least consecutive five T's, at least consecutive six T's, at least consecutive seven T's, at least consecutive eight T's, at least consecutive nine T's, at least consecutive ten T's, at least consecutive eleven T's, at least consecutive twelve T's, at least consecutive thirteen T's, at least consecutive fourteen T's, or at least consecutive fifteen T's should be avoided when designing the pegRNA, or should be at least removed from the final designed sequence. In one embodiment, one can avoid the includes of unwanted strings of consecutive T's in pegRNA extension arms but avoiding target sites that are rich in consecutive A:T nucleobase pairs.

In other embodiments, the prime editing system may include the use of pegRNA designs and strategies that can improve prime editing efficiency. These strategies seek to overcome some issues that exist because of the multi-step process required for prime editing. For example, unfavorable RNA structures that can form within the pegRNA can result in the inhibition of DNA edits being copied from the pegRNA into the genomic locus. These limitations could be overcome through the redesign and engineering of the pegRNA component. These redesigns could improve prime editor efficiency, and could allow the installation of longer inserted sequences into the genome.

Accordingly, in various embodiments, the pegRNA designs can result in longer pegRNAs by enabling efficient expression of functional pegRNAs from non-polymerase III (pol III) promoters, which would avoid the need for burdensome sequence requirements. In other embodiments, the core, Cas9-binding pegRNA scaffold can be improved to improve efficacy of the system. In yet other embodiments, modifications can be made to the pegRNA to improve reverse transcriptase (RT) processivity, which would enable the insertion of longer sequences at the targeted genomic loci. In other embodiments, RNA motifs can be added to the 5′ and/or 3′ termini of the pegRNA to improve stability, enhance RT processivity, prevent misfolding of the pegRNA, and/or recruit additional factors important for genome editing. In yet another embodiment, a platform is provided for the evolution of pegRNAs for a given sequence target that could improve the pegRNA scaffold and enhance prime editor efficiency. These designs could be used to improve any pegRNA recognized by any Cas9 or evolved variant thereof.

This application of prime editing can be further described in Example 2.

The pegRNAs may include additional design improvements that may modify the properties and/or characteristics of pegRNAs thereby improving the efficacy of prime editing. In various embodiments, these improvements may belong to one or more of a number of different categories, including but not limited to: (1) designs to enable efficient expression of functional pegRNAs from non-polymerase III (pol III) promoters, which would enable the expression of longer pegRNAs without burdensome sequence requirements; (2) improvements to the core, Cas9-binding pegRNA scaffold, which could improve efficacy; (3) modifications to the pegRNA to improve RT processivity, enabling the insertion of longer sequences at targeted genomic loci; and (4) addition of RNA motifs to the 5′ or 3′ termini of the pegRNA that improve pegRNA stability, enhance RT processivity, prevent misfolding of the pegRNA, or recruit additional factors important for genome editing.

In one embodiment, pegRNA could be designed with polIII promoters to improve the expression of longer-length pegRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed pegRNA, which has been shown to result in markedly reduced Cas9:sgRNA activity in a site-dependent manner. Additionally, while pol III-transcribed pegRNAs can simply terminate in a run of 6-7 U's, pegRNAs transcribed from pol II or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA- (lncRNA) tagged sgRNAs¹⁸³. These platforms include RNAs expressed from pCMV and that terminate in the ENE element from the MALAT1 ncRNA from humans¹⁸⁴, the PAN ENE element from KSHViss, or the 3′ box from U1 snRNA¹⁸⁶. Notably, the MALAT1 ncRNA and PAN ENEs form triple helices protecting the polyA-tail^{184, 187}. These constructs could also enhance RNA stability. It is contemplated that these expression systems will also enable the expression of longer pegRNAs.

In addition, a series of methods have been designed for the cleavage of the portion of the pol II promoter that would be transcribed as part of the pegRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy4¹⁹³ and also leads to processing of the guide. Also, incorporation of multiple ENE motifs can lead to improved pegRNA expression and stability. Circularizing the pegRNA in the form of a circular intronic RNA (ciRNA) can lead to enhanced RNA expression and stability, as well as nuclear localization.

In various embodiments, the pegRNA may include various above elements, as exemplified by SEQ ID NOs: 241-245.

In various other embodiments, the pegRNA may be improved by introducing improvements to the scaffold or core sequences. This can be done by introducing known

The core, Cas9-binding pegRNA scaffold can be improved to enhance PE activity. In an exemplary approach, the first pairing element of the scaffold (P1) contains a GTTTT-AAAAC (SEQ ID NO: 246) pairing element. Such runs of Ts can result in pol III pausing and premature termination of the RNA transcript. Rational mutation of one of the T-A pairs to a G-C pair in this portion of P1 can enhance sgRNA activity. This approach can be used to improve pegRNA. Additionally, increasing the length of P1 can enhance sgRNA folding and can improve pegRNA activity. Example improvements to the core can include:

pegRNA containing a 6 nt extension to P1
(SEQ ID NO: 228)
GGCCCAGACTGAGCACGTGAGTTTTAGAGCTAGCTCATGAAAATGAGCTA
GCAAGTTAAAATAAGGCTAGTCCGTTATCAACTTGAAAAAGTGGGACCGA
GTCGGTCCTCTGCCATCAAAGCGTGCTCAGTCTGTTTTTTT
pegRNA containing a T-A to G-C mutation within P1
(SEQ ID NOS: 247 AND 248
GGCCCAGACTGAGCACGTGAGTTTGAGAGCTAGAAATAGCAAGTTTAAAT
AAGGCTAGTCCGTTATCAACTTGAAAAAGTGGGACCGAGTCGGTCCTCTG
CCATCAAAGCGTGCTCAGTCTGTTTTTTT.

In various other embodiments, the pegRNA may be improved by introducing modifications to the edit template region. As the size of the insertion templated by the pegRNA increases, it is more likely to be degraded by endonucleases, undergo spontaneous hydrolysis, or fold into secondary structures unable to be reverse-transcribed by the RT or that disrupt folding of the pegRNA scaffold and subsequent Cas9-RT binding. Accordingly, it is likely that modification to the template of the pegRNA might be necessary to affect large insertions, such as the insertion of whole genes. Some strategies to do so include the incorporation of modified nucleotides within a synthetic or semi-synthetic pegRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the pegRNA to enhance stability and activity. Alternatively or additionally, the template of the pegRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate pegRNAs. In such a design, a PE would be used to initiate transcription and also recruit a separate template RNA to the targeted site via an RNA-binding protein fused to Cas9 or an RNA recognition element on the pegRNA itself such as the MS2 aptamer. The RT could either directly bind to this separate template RNA, or initiate reverse transcription on the original pegRNA before swapping to the second template. Such an approach could enable long insertions by both preventing misfolding of the pegRNA upon addition of the long template and also by not requiring dissociation of Cas9 from the genome for long insertions to occur, which could possibly be inhibiting PE-based long insertions.

In still other embodiments, the pegRNA may be improved by introducing additional RNA motifs at the 5′ and 3′ termini of the pegRNAs. Several such motifs—such as the PAN ENE from KSHV and the ENE from MALAT1 were discussed above as possible means to terminate expression of longer pegRNAs from non-pol III promoters. These elements form RNA triple helices that engulf the polyA tail, resulting in their being retained within the nucleus^{184,187}. However, by forming complex structures at the 3′ terminus of the pegRNA that occlude the terminal nucleotide, these structures would also likely help prevent exonuclease-mediated degradation of pegRNAs.

Other structural elements inserted at the 3′ terminus could also enhance RNA stability, albeit without enabling termination from non-pol III promoters. Such motifs could include hairpins or RNA quadruplexes that would occlude the 3′ terminus¹⁹⁷, or self-cleaving ribozymes such as HDV that would result in the formation of a 2′-3′-cyclic phosphate at the 3′ terminus and also potentially render the pegRNA less likely to be degraded by exonucleases¹⁹⁸. Inducing the pegRNA to cyclize via incomplete splicing—to form a ciRNA—could also increase pegRNA stability and result in the pegRNA being retained within the nucleus¹⁹⁴.

Additional RNA motifs could also improve RT processivity or enhance pegRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹.

Addition of dimerization motifs—such as kissing loops or a GNRA tetraloop/tetraloop receptor pair²⁰⁰—at the 5′ and 3′ termini of the pegRNA could also result in effective circularization of the pegRNA, improving stability. Additionally, it is envisioned that addition of these motifs could enable the physical separation of the pegRNA spacer and primer, preventing occlusion of the spacer which could hinder PE activity. Short 5′ or 3′ extensions to the pegRNA that form a small toehold hairpin in the spacer region could also compete favorably against the annealing region of the pegRNA binding the spacer. Finally, kissing loops could also be used to recruit other template RNAs to the genomic site and enable swapping of RT activity from one RNA to the other. Example improvements include, but are not limited to SEQ ID NOs: 251-255.

pegRNA scaffold could be further improved via directed evolution, in an analogous fashion to how SpCas9 and prime editor (PE) have been improved. Directed evolution could enhance pegRNA recognition by Cas9 or evolved Cas9 variants. Additionally, it is likely that different pegRNA scaffold sequences would be optimal at different genomic loci, either enhancing PE activity at the site in question, reducing off-target activities, or both. Finally, evolution of pegRNA scaffolds to which other RNA motifs have been added would almost certainly improve the activity of the fused pegRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-pegRNA fusions as well. In addition, while Cas9 currently does not generally tolerate 5′ extension of the sgRNA, directed evolution will likely generate enabling mutations that mitigate this intolerance, allowing additional RNA motifs to be utilized.

The present disclosure contemplates any such ways to further improve the efficacy of the prime editing systems disclosed here.

### [7] Computational Method for Nucleotide Linker Design

In one aspect of the disclosure, the inventors have developed a novel computational technique, which may be embodied in software, for identifying one or more nucleotide linkers for coupling a prime editing guide RNA to a nucleic acid moiety, such as, but not limited to, an aptamer (e.g., prequeosin₁-1 riboswitch aptamer or “evopreQ₁-1”) or a variant thereof, a pseudoknot (the MMLV viral genome pseudoknot or “Mpknot-1”) or a variant thereof, a tRNA (e.g., the modified tRNA used by MMLV as a primer for reverse transcription) or a variant thereof, or a G-quadruplex or a variant thereof. Exemplary nucleotide sequences of such linkers are provided throughout herein, and include, but are not limited to SEQ ID NOs: 225-236.

The computational technique, which may be referred to herein as the pegRNA Linker Identification Tool (“pegLIT”), involves efficiently evaluating nucleic acid linker candidates to identify those which have lower propensity for base pairing to other regions of the pegRNA (e.g., regions comprising the primer binding site, spacer, DNA synthesis template, and/or gRNA core). In some embodiments, the propensity of a particular linker candidate to base pair with one or more regions of the pegRNA may be determined using computational tools for modeling RNA-to-RNA interactions while taking into account RNA's secondary structure. One illustrative example of such a computational tool is ViennaRNA, aspects of which are described in Lorenz, R. et al. ViennaRNA package 2.0. Algorithms Mol Biol 6, 26 (2011), which is incorporated by reference herein in its entirety.

The inventors have recognized that evaluating the fitness of each possible nucleic acid linker candidate is computationally impractical because: (1) the evaluation of fitness of a single linker candidate is computationally expensive (e.g., because it involves physics-based modeling of RNA secondary structures); and (2) the number of linker candidates to be considered increases exponentially with their length. Moreover, in the context of screening, all linker candidates would have to be re-evaluated for any change in the pegRNA (e.g., in the PBS, spacer, template, and/or core regions).

Accordingly, the inventors have developed an optimization technique to efficiently explore the space of nucleic acid linker candidates to identify linkers suitable for coupling a pegRNA to a nucleic acid moiety. In some embodiments, the optimization technique involves using an iterative optimization approach (e.g., simulated annealing) to identify a number of linker candidates. In some embodiments, the linker candidates identified using the optimization technique may be clustered to obtain linker clusters and one or more representative linkers in each cluster may be returned, which may help to promote diversity among the identified linkers.

In some embodiments, the optimization technique involves calculated multiple scores for each linker candidate being considered. Each of the multiple scores may be indicative of a degree to which the linker candidate may interact with a region of the pegRNA. In this way, the multiple scores for a single linker candidate represent the degree to which the linker candidate interacts with multiple regions of the pegRNA. Considering linker-to-pegRNA interactions on a region-by-region basis helps to determine the fitness of each linker candidate more accurately than is possible by other methods.

Indeed, the computational technique developed by the inventors not only results in computational improvements (e.g., a reduction in the utilization of processor and memory computer resources) relative to a brute-force search approach, but also identifies linkers that improve overall PE editing efficiency as compared with human-designed linkers or linkers that were predicted by the computational tool to interact with the primer binding site. The improvements in editing efficiency are shown in and described with reference to FIGS. 113A-113E.

Accordingly, some embodiments provide for a method for identifying at least one nucleic acid linker for coupling a prime editing guide RNA (pegRNA) to a nucleic acid moiety, the method comprising: (1) generating a plurality of nucleic acid linker candidates including a first nucleic acid linker candidate; (2) identifying the at least one nucleic acid linker from among the plurality of nucleic acid linker candidates at least in part by: (a) calculating multiple scores for each of at least some of the plurality of nucleic acid linker candidates, the calculating comprising calculating a first set of scores for the first nucleic acid linker candidate, the first set of scores comprising: a first score indicative of a degree of interaction between the first nucleic acid linker candidate and a first region of the pegRNA; a second score indicative of a degree of interaction between the first nucleic acid linker candidate and a second region of the pegRNA (e.g., with the first and second regions being different regions); and (b) identifying the at least one nucleic acid linker from among the at least some of the plurality of nucleic acid linker candidates using the calculated multiple scores; and (3) outputting information indicative of the at least one nucleic acid linker.

In some embodiments, the first score is indicative of a degree to which the first nucleic acid linker candidate is predicted to avoid interaction with the first region of the pegRNA, and the second score is indicative of a degree to which the first nucleic acid linker candidate is predicted to avoid interaction with the second region of the pegRNA. For example, if the first score has the value of 0.8, that may indicate that, on average, the predicted probability of a pegRNA folded state lacking base pairing between any nucleotide of the linker candidate and the first region is 80%. As another example, if the second score has the value of 0.9, that may indicate that, on average, the predicted probability of a pegRNA folded state lacking base pairing between any nucleotide of the linker candidate and the second region is 90%.

In some embodiments, the first region may comprise a primer binding site of the pegRNA, a spacer of the pegRNA, a DNA synthesis template of the pegRNA, or a gRNA core of the pegRNA. The second region also may comprise a primer binding site of the pegRNA, a spacer of the pegRNA, a DNA synthesis template of the pegRNA, or a gRNA core of the pegRNA.

In some embodiments, the first set of scores further comprises a third score indicative of a degree to which the first nucleic acid linker candidate is predicted to avoid interaction with a third region of the pegRNA and a fourth score indicative of a degree to which the first nucleic acid linker candidate is predicted to avoid interaction with a fourth region of the pegRNA. In some embodiments, the first, second, third, and fourth regions may comprise, respectively, a PBS of the pegRNA, a spacer of the pegRNA, a DNA synthesis template of the pegRNA, and a gRNA core of the pegRNA.

In some embodiments, the pegRNA is for installing a nucleotide edit in a double stranded target DNA sequence. The pegRNA may comprise: a spacer that hybridizes to a first strand of the double stranded target DNA sequence, an extension arm that hybridizes to a second strand of the double stranded target DNA sequence, the extension arm comprising a primer binding site (PBS) and a DNA synthesis template comprising the nucleotide edit, and a gRNA core that interacts with a nucleic acid programmable DNA binding protein napDNAbp. In some such embodiments, the first region comprises the PBS, the second region comprises the spacer, the third region comprises the DNA synthesis template, and the fourth region comprises the gRNA core.

In some embodiments, fitness of various linker candidates may be evaluated relative to one another based on their scores. In some embodiments, the plurality of nucleic acid linker candidates comprises a second nucleic acid linker candidate, and identifying the at least one nucleic acid linker from among the at least some of the plurality of nucleic acid linker candidates using the calculated multiple scores comprises comparing the first set of scores for the first nucleic acid linker candidate with a second set of scores for the second nucleic acid linker candidate.

There are multiple ways in which the score sets of two different linker candidates may be compared. For example, in some embodiments, each score set may have constituent scores for certain regions (e.g., the region comprising PBS, the region comprising spacer) and the candidates may be compared first based on their respective scores for a particular region (e.g., the region comprising the PBS). If the scores for the particular region are equal or are within a threshold (e.g., such that they may be considered to be close to one another), then scores for another region may be compared (e.g., the region comprising the spacer). If the scores for the other region are equal or are within a threshold, the scores for a third region (e.g., the region comprising the DNA synthesis template) may be compared. If the scores for the third region are equal or are within a threshold, the scores for a fourth region (e.g., the region comprising the gRNA core) may be compared. And so on.

Accordingly, in some embodiments, the first region comprises a primer binding site (PBS), the first score in the first set of scores is indicative of a degree to which the first nucleic acid linker candidate is predicted to avoid interaction with the first region of the pegRNA, a third score in the second set of scores is indicative of a degree to which the second nucleic acid linker candidate is predicted to avoid interaction with the first region of the pegRNA, and comparing the first set of scores with the second set of scores comprises comparing the first score with the third score. In some embodiments, when the first score is equal to or is within a threshold distance of the third score, comparing the first set of scores with the second set of scores further comprises comparing a score, other than the first score, in the first set of scores with another score, other than the third score, in the second set of scores.

In some embodiments, the technique for identifying candidate linkers may be performed iteratively. In some embodiments, generating the plurality of nucleic acid linker candidates and determining the at least one nucleic acid linker from among the plurality of nucleic acid linker candidates may be performed in accordance with an iterative optimization algorithm (e.g., one that involves simulating annealing).

In some embodiments, the plurality of nucleic acid linker candidates includes a second nucleic acid linker candidate, and performing the generating and the determining in accordance with the iterative optimization algorithm comprises: generating the first nucleic acid linker candidate; determining the first scores for the first nucleic acid linker candidate, wherein the identifying comprises determining whether to include the first nucleic acid linker candidate in the at least one nucleic acid linker based on the first scores; after determining the first scores for the first nucleic acid linker candidate, generating the second acid linker candidate; and determining second scores for the second nucleic acid linker candidate, wherein the identifying comprises determining whether to include the second nucleic acid linker candidate in the at least one nucleic acid linker based on the second scores. Aspects of such an iterative embodiment are described herein including with reference to FIG. 119.

In some embodiments, computing scores in the first set of scores is performed using software for modeling RNA-to-RNA interactions (e.g., ViennaRNA).

In some embodiments, the techniques further include filtering the plurality of nucleic acid linker candidates using one or more filtering rules. For example, linker candidates having at least a threshold number of the same nucleotide appearing consecutively (e.g., four uridines in a row) may be removed from further consideration in accordance with a filtering rule. As another example, linker candidates with AC content below a threshold percentage (e.g., below 50%) may be removed from further consideration in accordance with a filtering rule. One or more other filtering rules may be used in addition to or instead of the above two example filtering rules, as aspects of the technology described herein are not limited in this respect.

In some embodiments, the techniques further include clustering identified linker candidates and determining linkers representative of different clusters in order to obtain a diverse population of linker candidates. Accordingly, in some embodiments, identifying the at least one nucleic acid linker comprises: identifying a subset of the plurality of nucleic acid linker candidates based on their respective scores; clustering (e.g., using hierarchical agglomerative clustering or any other suitable clustering technique) the subset of nucleic acid linker candidates to obtain a plurality of clusters; and including at least one representative member of each of the plurality of clusters in the at least one nucleic acid linker.

In some embodiments, one or more of the at least one nucleic acid linker identified may be manufactured and used in various applications described herein.

FIG. 118 is a flowchart of an illustrative process 11800 for identifying one or more nucleic acid linkers for coupling a prime editing guide RNA to a nucleic acid moiety, in accordance with some embodiments of the technology described herein. The process 11800 may be implemented using any suitable computing device(s), as aspects of the technology described herein are not limited in this respect.

Process 11800 begins at act 11802 where one or more nucleic acid linker candidates may be generated. Any suitable number of linker candidates may be generated at act 11802 in any suitable way. In some embodiments, multiple linker candidates may be generated at act 11802 and some or all of these candidates may be further evaluated at act 11810 and its subacts. In other embodiments, one or a small number of linker candidates may be generated at act 11802 and, when it is determined that additional linker candidates are needed (e.g., at act 11818), then process 11800 may return to act 11802 so that additional linker candidates are generated.

Each of the linkers generated may have any suitable length. For example, a linker candidate may consist of 4 nucleotides, 8 nucleotides, 15 nucleotides, or any suitable number of nucleotides in the ranges of 4-32 nucleotides, 8-16 nucleotides, or any other suitable range within these ranges.

In some embodiments, a linker candidate may be generated by selecting each of one or more (or all) of the nucleotides at random. Each nucleotide may be selected uniformly at random or in accordance with a specified distribution (e.g., uniform or any other discrete distribution). In some embodiments, each of the nucleotides may be selected independently of the other linker nucleotides. In some embodiments, two or more of the nucleotides may be selected in a correlated way, for example, by sampling from joint distribution defined on a sequence of two or more nucleotides (whether or not consecutive).

Next, process 11800 proceeds to act 11810, where at least one nucleic acid linker is identified from among the nucleic acid linker candidates generated during act 11802. The identification involves: (1) at act 11812, calculating multiple scores for each of at least some of the linker candidates generated during act 11802; and (2) at act 11818, identifying the at least one nucleic acid linker candidate using the multiple scares calculated at act 11812. Each of these acts is described in turn. In some embodiments, the linker candidates may be filtered using one or more filtering rules (examples of which are described herein) so that there is no need to expend computing resources to calculate scores for linker candidates that are otherwise unsuitable.

As described herein, calculating multiple scores for a particular linker candidate involves determining multiple scores for a respective plurality of multiple regions of pegRNA. In some embodiments, each of the multiple scores may be indicative of a degree of interaction between the linker candidate and a respective one of the multiple regions. For example, a particular score may be indicative of a degree to which the linker is predicted to interact with or to avoid interaction with a particular region. In the illustrative example, act 11812 involves calculating at least two scores for each of at least some of linker candidates. In particular, at act 11814, a first score indicative of a degree of interaction between a first linker candidate and a first region of the pegRNA (e.g., a region comprising the PBS or any other suitable region examples of which are provided herein) may be calculated and, at act 11816, a second score indicative of a degree of interaction between the first linker candidate the a second region of the pegRNA (e.g., a region comprising the spacer or any other suitable region examples of which are provided herein) may be calculated. Each of the scores may be calculated using RNA-to-RNA interaction modeling software (e.g., ViennaRNA) or any other suitable software, as aspects of the technology described herein are not limited in this respect.

Although in the illustrative example act 11812 involves calculating two scores, in some embodiments 3, 4, 5, 6, or any other suitable number of scores may be calculated for each linker candidate to obtain a measure of a degree of interaction with 3, 4, 5, 6, or any other suitable number of pegRNA regions, as aspects of the technology described herein are not limited in this respect. For example, in one illustrative embodiment, four scores may be calculated for a linker candidate and may be indicative of a degree of interaction between the linker candidate and the PBS, spacer, DNA synthesis template, and gRNA core regions of the pegRNA.

At act 11818, the calculated scores may be used to identify the “best” nucleic acid linker candidates. For example, the scores may be used to identify a subset of the linker candidates that are predicted to have the least interaction with one or more regions of the pegRNA. Interaction with some regions of the pegRNA may be considered to be worse than interaction with other regions of the pegRNA. Accordingly, in some embodiments, the scores may be examined on a per region basis to identify linker candidates for subsequent use. For example, in some embodiments, the linker candidates may be compared based on their PBS scores—scores indicating a degree to which the linkers are predicted to avoid interacting with a region of the pegRNA comprising the PBS. A threshold number of candidate linkers interacting least with such a region may be retained (e.g., 100 linker candidates that are predicted to interact least with the PBS may be retained). Should multiple linker candidates have the same score for the same region, these candidates may be compared/ranked with using their scores for other regions, as described herein.

In some embodiments, the acts 11812 and 11818 may be performed in accordance with an iterative optimization algorithm (as indicated by the arrow from 11818 to 11812 in FIG. 118). For example, the acts 11812 and 11818 may be performed in accordance with a simulated annealing technique. Aspects of this are described herein including with reference to FIG. 119.

After a subset of the nucleic acid candidates are identified based on their corresponding scores (and there may be any suitable number of such candidates identified; for example, this may be controlled by a parameter setting indicating the desired number of candidates), in some embodiments, the identified nucleic acid linker candidates may be further sieved to identify a subset of linker candidates which are diverse in their sequence makeup. To this end, in some embodiments, the identified nucleic acid linker candidates may be clustered to obtain multiple clusters and one or more representative linkers in each cluster may be output at act 11818, which promotes sequence diversity among the output linker candidates. Any suitable clustering technique (e.g., agglomerative hierarchical clustering) may be used for this, as aspects of the technology described herein are not limited in this respect.

Information about the linker candidates identified during act 11810 may be output at act 11820. The information may include the sequences of the linker candidates, their scores, and/or any other related information. The information may be transmitted to one or more other computing devices over a communication network, stored in at least one non-transitory computer readable storage medium (e.g., in memory, on a hard drive, in a file, etc.) for subsequent access, presented in a graphical user interface, and/or output in any other suitable way.

As described above, aspects of the process 18000 may be performed iteratively. One illustrative example of this is shown in FIG. 119. FIG. 119 is a flowchart of an illustrative process 11900 for iteratively identifying one or more nucleic acid linkers for coupling a prime editing guide RNA to a nucleic acid moiety, in accordance with some embodiments of the technology described herein. The process 11900 may be implemented using any suitable computing device(s), as aspects of the technology described herein are not limited in this respect.

Process 11900 begins at act 11902, where a nucleic acid linker candidate is generated. The linker candidate may have any suitable length, and examples of lengths are provided herein. The linker candidate may be generated in any suitable way including in any of the ways described with reference to act 11802.

Next, process 11900 proceeds to decision block 11904, where it is determined whether the linker candidate generated at act 11902 passes one or more filtering rules. Any suitable filtering rules may be used to eliminate unwanted linker candidates. For example, a candidate linker having at least a threshold number of the same nucleotide appearing consecutively (e.g., four uridines in a row) may be removed from further consideration (by not passing this filtering rule). As another example, a candidate linker having AC content below a threshold percentage (e.g., below 50%) may be removed from further consideration (by not passing this filtering rule).

When it is determined, at decision block 11904, that the linker candidate does not pass one or more filter rules, the process 11900 returns to act 11902, where another linker candidate is generated. On the other hand, when it is determined, at decision block 11904, that the linker candidate passes the filter rule(s), process 11900 proceeds to act 11906, where multiple scores for the linker candidate are calculated. As described herein, the multiple scores indicate, for respective multiple pegRNA regions, a degree of interaction between the candidate linker and the regions. Aspects of how to calculate the multiple scores for a linker candidate are described herein.

Next, process 11900 proceeds to act 11908, where the scores for the linker candidate that were determined at act 11906 are compared with scores for linker candidates that were previously retained. For example, a set of linker candidates (e.g., 100 candidates) may have already been identified from among the candidate linkers examined so far and the scores for the new linker candidate (the candidate generated at act 11902) may be compared to the previously determined scores for the retained candidates. Aspects of how to compare the scores of a linker candidate with respective scores of other linker candidates are described herein.

On the basis of this comparison, at decision block 11910, it is determine whether the new linker candidate is to be retained. When the comparison of act 11908 indicates that the new linker candidate is better than at least one of the retained candidates, then the new linker candidate may be retained, at act 11912. Optionally, one of the previously retained linker candidates may be dropped from the list (e.g., if there is a fixed number of linker candidates that may be retained and adding a new linker to the list causes the total number of retained linker candidates to exceed the fixed number).

On the other hand, when the comparison of act 11908 indicates that the new linker candidate is not better than any of the retained candidates, then the new linker candidate is retained only with a certain probability at act 11912; otherwise it is dropped and process 11900 returns to act 11902 where a new linker candidate may be generated. That probability may be selected in accordance with a simulated annealing schedule, in some embodiments. In that sense, the iterative optimization scheme of process 11900 may be considered to involve simulated annealing.

After the linker candidate is retained at act 11912, process 11900 proceeds to decision block 11914 where it is determined whether additional linker candidates should be generated. This determination may be made in any suitable way, for example, based on the number of iterations/time taken, on how many candidate linkers have been retained, on an estimate of quality and/or diversity among the retained candidates, and/or any suitable metric. When it is determined that additional linker candidates are to be generated, the process 11900 returns to act 11902 where a new linker candidate may be generated. Otherwise, the process 11900 proceeds to act 11916 where information indicative of at least some (e.g., all, at least one representative member of clusters of all) of the retained linker candidates is output. Examples of outputting information about retained candidate linkers is described herein.

FIG. 120 shows an illustrative implementation of a computer system 12000 in which embodiments of the technology described herein may be implemented. For example, any of the computing devices described herein may be implemented as computing system 12000. The computing system 12000 may include one or more computer hardware processors 12002 and one or more articles of manufacture that comprise non-transitory computer-readable storage media (e.g., memory 12004 and one or more non-volatile storage devices 12006). The processor 12002(s) may control writing data to and reading data from the memory 12004 and the non-volatile storage device(s) 12006 in any suitable manner. To perform any of the functionality described herein, the processor(s) 12002 may execute one or more processor-executable instructions stored in one or more non-transitory computer-readable storage media (e.g., the memory 12004), which may serve as non-transitory computer-readable storage media storing processor-executable instructions for execution by the processor(s) 12002.

The terms “program” or “software” are used herein in a generic sense to refer to any type of computer code or set of processor-executable instructions that may be employed to program a computer or other processor to implement various aspects of embodiments as described above. Additionally, according to one aspect, one or more computer programs that when executed perform methods of the disclosure provided herein need not reside on a single computer or processor but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements);etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.[9] Split pegRNA designs for trans prime editing

The instant disclosure also contemplates trans prime editing, which refers to a modified version of prime editing which operates by separating the pegRNA into two distinct molecules: a guide RNA and a tPERT molecule. The tPERT molecule is programmed to co-localize with the prime editor complex at a target DNA site, bringing the primer binding site and the DNA synthesis template to the prime editor in trans. For example, see FIG. 3G for an embodiment of a trans prime editor (tPE) which shows a two-component system comprising (1) an recruiting protein (RP)-PE:gRNA complex and (2) a tPERT that includes a primer binding site and a DNA synthesis template joined to an RNA-protein recruitment domain (e.g., stem loop or hairpin), wherein the recruiting protein component of the RP-PE:gRNA complex recruits the tPERT to a target site to be edited, thereby associating the PBS and DNA synthesis template with the prime editor in trans. Said another way, the tPERT is engineered to contain (all or part of) the extension arm of a pegRNA, which includes the primer binding site and the DNA synthesis template. One advantage of this approach is to separate the extension arm of a pegRNA from the guide RNA, thereby minimizing annealing interactions that tend to occur between the PBS of the extension arm and the spacer sequence of the guide RNA.

Trans prime editing may be conducts with any pegRNA described herein, including the modified pegRNAs described herein which result in improved PE editing efficiency.

A key feature of trans prime editing is the ability of the trans prime editor to recruit the tPERT to the site of DNA editing, thereby effectively co-localizing all of the functions of a pegRNA at the site of prime editing. Recruitment can be achieve by installing an RNA-protein recruitment domain, such as a MS2 aptamer, into the tPERT and fusing a corresponding recruiting protein to the prime editor (e.g., via a linker to the napDNAbp or via a linker to the polymerase) that is capable of specifically binding to the RNA-protein recruitment domain, thereby recruiting the tPERT molecule to the prime editor complex. As depicted in the process described in FIG. 3H, the RP-PE:gRNA complex binds to and nicks the target DNA sequence. Then, the recruiting protein (RP) recruits a tPERT to co-localize to the prime editor complex bound to the DNA target site, thereby allowing the primer binding site, located on the tPERT, to bind to the primer sequence on the nicked strand, and subsequently, allowing the polymerase (e.g., RT) to synthesize a single strand of DNA against the DNA synthesis template, located on the tPERT, up through the 5′ end of the tPERT.

While the tPERT is shown in FIG. 3G and FIG. 3H as comprising the PBS and DNA synthesis template on the 5′ end of the RNA-protein recruitment domain, the tPERT in other configurations may be designed with the PBS and DNA synthesis template located on the 3′ end of the RNA-protein recruitment domain. However, the tPERT with the 5′ extension has the advantage that synthesis of the single strand of DNA will naturally terminate at the 5′ end of the tPERT and thus, does not risk using any portion of the RNA-protein recruitment domain as a template during the DNA synthesis stage of prime editing.

### [8] Delivery of Prime Editors

In another aspect, the present disclosure provides for the delivery of prime editors in vitro and in vivo using various strategies, including on separate vectors using split inteins and as well as direct delivery strategies of the ribonucleoprotein complex (i.e., the prime editor complexed to the pegRNA and/or the second-site gRNA) using techniques such as electroporation, use of cationic lipid-mediated formulations, and induced endocytosis methods using receptor ligands fused to the ribonucleoprotein complexes. Any such methods are contemplated herein.

### Overview of Delivery Options

In some aspects, the invention provides methods comprising delivering one or more prime editor-encoding polynucleotides, such as or one or more vectors as described herein encoding one or more components of the prime editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a prime editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a prime editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and W2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In various embodiments, the PE constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split Prime editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VPlu, which has the genome of AAV2, capsid backbone of AAV5 and VPlu of AAV1. Other non-limiting example of derivatives and pseudotypes that have chimeric VP1 proteins are rAAV2/5-8VPlu, rAAV2/9-1VPlu, and rAAV2/9-8VPlu.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VPlu region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the pegRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the pegRNA.

In various embodiments, the pegRNAs and the second-site nicking guide RNAs can be expressed from an appropriate promoter, such as a human U6 (hU6) promoter, a mouse U6 (mU6) promoter, or other appropriate promoter. The pegRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle or the herein compositions are administered to the subject by injection into the hepatic artery or portal vein.

### Split PE Vector-Based Strategies

In this aspect, the prime editors can be divided at a split site and provided as two halves of a whole/complete prime editor. The two halves can be delivered to cells (e.g., as expressed proteins or on separate expression vectors) and once in contact inside the cell, the two halves form the complete prime editor through the self-splicing action of the inteins on each prime editor half. Split intein sequences can be engineered into each of the halves of the encoded prime editor to facilitate their transplicing inside the cell and the concomitant restoration of the complete, functioning PE.

These split intein-based methods overcome several barriers to in vivo delivery. For example, the DNA encoding prime editors is larger than the rAAV packaging limit, and so requires special solutions. One such solution is formulating the editor fused to split intein pairs that are packaged into two separate rAAV particles that, when co-delivered to a cell, reconstitute the functional editor protein. Several other special considerations to account for the unique features of prime editing are described, including the optimization of second-site nicking targets and properly packaging prime editors into virus vectors, including lentiviruses and rAAV.

In this aspect, the prime editors can be divided at a split site and provided as two halves of a whole/complete prime editor. The two halves can be delivered to cells (e.g., as expressed proteins or on separate expression vectors) and once in contact inside the cell, the two halves form the complete prime editor through the self-splicing action of the inteins on each prime editor half. Split intein sequences can be engineered into each of the halves of the encoded prime editor to facilitate their transplicing inside the cell and the concomitant restoration of the complete, functioning PE.

FIG. 66 depicts one embodiment of a prime editor being provided as two PE half proteins which regenerate as whole prime editor through the self-splicing action of the split-intein halves located at the end or beginning of each of the prime editor half proteins. As used herein, the term “PE N-terminal half” refers to the N-terminal half of a complete prime editor and which comprises the “N intein” at the C-terminal end of the PE N-terminal half (i.e., the N-terminal extein) of the complete prime editor. The “N intein” refers to the N-terminal half of a complete, fully-formed split-intein moiety. As used herein, the term “PE C-terminal half” refers to the C-terminal half of a complete prime editor and which comprises the “C intein” at the N-terminal end of the C-terminal half (i.e., the C-terminal extein) of a complete prime editor. When the two half proteins, i.e., the PE N-terminal half and the PE C-terminal half, come into contact with one another, e.g., within the cell, the N intein and the C intein undergo the simultaneous process of self-excision and the formation of a peptide bond between the C-terminal end of the PE N-terminal half and the N-terminal end of the PE C-terminal half to reform the complete prime editor protein comprising the complete napDNAbp domain (e.g., Cas9 nickase) and the RT domain. Although not shown in the drawing, the prime editor may also comprise additional sequences including NLS at the N-terminus and/or C-terminus, as well as amino acid linkers sequences joining each domain.

In various embodiments, the prime editors may be engineered as two half proteins (i.e., a PE N-terminal half and a PE C-terminal half) by “splitting” the whole prime editor as a “split site.” The “split site” refers to the location of insertion of split intein sequences (i.e., the N intein and the C intein) between two adjacent amino acid residues in the prime editor. More specifically, the “split site” refers to the location of dividing the whole prime editor into two separate halves, wherein in each halve is fused at the split site to either the N intein or the C intein motifs. The split site can be at any suitable location in the prime editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

In some embodiments, the split site is located in the napDNAbp domain. In other embodiments, the split site is located in the RT domain. In other embodiments, the split site is located in a linker that joins the napDNAbp domain and the RT domain.

In various embodiments, split site design requires finding sites to split and insert an N- and C-terminal intein that are both structurally permissive for purposes of packaging the two half prime editor domains into two different AAV genomes. Additionally, intein residues necessary for trans splicing can be incorporated by mutating residues at the N terminus of the C terminal extein or inserting residues that will leave an intein “scar.”

Exemplary split configurations of split prime editors comprising either the SpCas9 nickase or the SaCas9 nickase are as follows.

S. PYOGENES PE, SPLIT AT LINKER, N TERMINAL PORTION
STRUCTURE: [N EXTEIN]-[N INTEIN]
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYF
TVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGG
DSGGSSGGSCLSYETEILTVEYGLLPIGKIVEKRIECTVYSVDNNGNIYTQPVAQWH
DRGEQEVFEYCLEDGSLIRATKDHKFMTVDGQMLPIDEIFERELDLMRVDNLPNSG
GSKRTADGSEFEPKKKRKV(SEQ ID NO: 381)
KEY:
NLS (SEQ ID NO: 29, 155)
CAS9 (SEQ ID NO: 31)
LINKER (SEQ ID NO: 9)
NPUN INTEIN (SEQ ID NO: 382)
S. PYOGENES PE, SPLIT AT LINKER, C TERMINAL PORTION
STRUCTURE: [C INTEIN]-[C EXTEIN]
MKRTADGSEFESPKKKRKVIKIATRKYLGKQNVYDIGVERDHNFALKNGFIASNCFNS
GSETPGTSESATPESSGGSSGGSSTLNIEDEYRLHETSKEPDVSLGSTWLSDFPQAWA
ETGGMGLAVRQAPLIIPLKATSTPVSIKQYPMSQEARLGIKPHIQRLLDQGILVPCQSPW
NTPLLPVKKPGTNDYRPVQDLREVNKRVEDIHPTVPNPYNLLSGLPPSHQWYTVLDLKD
AFFCLRLHPTSQPLFAFEWRDPEMGISGQLTWTRLPQGFKNSPTLFDEALHRDLADFRI
QHPDLILLQYVDDLLLAATSELDCQQGTRALLQTLGNLGYRASAKKAQICQKQVKYLGY
LLKEGQRWLTEARKETVMGQPTPKTPRQLREFLGTAGFCRLWIPGFAEMAAPLYPLTKT
GTLFNWGPDQQKAYQEIKQALLTAPALGLPDLTKPFELFVDEKQGYAKGVLTQKLGPW
RRPVAYLSKKLDPVAAGWPPCLRMVAAIAVLTKDAGKLTMGQPLVILAPHAVEALVKQP
PDRWLSNARMTHYQALLLDTDRVQFGPVVALNPATLLPLPEEGLQHNCLDILAEAHGTR
PDLTDQPLPDADHTWYTDGSSLLQEGQRKAGAAVTTETEVIWAKALPAGTSAQRAELIA
LTQALKMAEGKKLNVYTDSRYAFATAHIHGEIYRRRGLLTSEGKEIKNKDEILALLKALFL
PKRLSIIHCPGHQKGHSAEARGNRMADQAARKAAITETPDTSTLLIENSSPSGGSKRTA
DGSEFEPKKKRKV(SEQ ID NO: 383)
KEY:
NLS (SEQ ID NO: 29, 155)
LINKER 1 (SEQ ID NO: 384)
LINKER 2 (SEQ ID NO: 8)
NPUC INTEIN (SEQ ID NO: 385)
RT (SEQ ID NO: 32)
S. AUREUS PE, SPLIT BETWEEN RESIDUES 740/741, N TERMINAL
PORTION STRUCTURE: [N EXTEIN]-[N INTEIN]
MKRTADGSEFESPKKKRKVGKRNYILGLAIGITSVGYGIIDYETRDVIDAGVRLF
KEANVENNEGRRSKRGARRLKRRRRHRIQRVKKLLFDYNLLTDHSELSGINP
YEARVKGLSQKLSEEEFSAALLHLAKRRGVHNVNEVEEDTGNELSTKEQISRN
SKALEEKYVAELQLERLKKDGEVRGSINRFKTSDYVKEAKQLLKVQKAYHQL
DQSFIDTYIDLLETRRTYYEGPGEGSPFGWKDIKEWYEMLMGHCTYFPEELR
SVKYAYNADLYNALNDLNNLVITRDENEKLEYYEKFQIIENVFKQKKKPTLKQ
IAKEILVNEEDIKGYRVTSTGKPEFTNLKVYHDIKDITARKEIIENAELLDQIAKI
LTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAII
KKYGLPNDIIIELAREKNSKDAQKMINEMQKRNRQTNERIEEIIRTTGKENAK
YLIEKIKLHDMQEGKCLYSLEAIPLEDLLNNPFNYEVDHIIPRSVSFDNSFNNKV
LVKQEENSKKGNRTPFQYLSSSDSKISYETFKKHILNLAKGKGRISKTKKEYLL
EERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQ
MFEEKQAECLSYETEILTVEYGLLPIGKIVEKRIECTVYSVDNNGNIYTQPVAQWH
DRGEQEVFEYCLEDGSLIRATKDHKFMTVDGQMLPIDEIFERELDLMRVDNLPNSG
GSKRTADGSEFEPKKKRKV(SEQ ID NO: 386)
KEY:
NLS (SEQ ID NO: 29, 155)
CAS9 (SEQ ID NO: 387)
LINKER (SEQ ID NO: 8)
NPUN INTEIN (SEQ ID NO: 388)
S. AUREUS PE, SPLIT BETWEEN RESIDUES 740/741, C TERMINAL
PORTION STRUCTURE: [C INTEIN]-[C EXTEIN]
MKRTADGSEFESPKKKRKVIKIATRKYLGKQNVYDIGVERDHNFALKNGFIASNCFNE
IETEQEYKEIFITPHQIKHIKDFKDYKYSHRVDKKPNRKLINDTLYSTRKDDKG
NTLIVNNLNGLYDKDNDKLKKLINKSPEKLLMYHHDPQTYQKLKLIMEQYGD
EKNPLYKYYEETGNYLTKYSKKDNGPVIKKIKYYGNKLNAHLDITDDYPNSRN
KVVKLSLKPYRFDVYLDNGVYKFVTVKNLDVIKKENYYEVNSKCYEEAKKLK
KISNQAEFIASFYKNDLIKINGELYRVIGVNNDLLNRIEVNMIDITYREYLENMN
DKRPPHIIKTIASKTQSIKKYSTDILGNLYEVKSKKHPQIIKKGSGGSSGGSSGS
ETPGTSESATPESSGGSSGGSSTLNIEDEYRLHETSKEPDVSLGSTWLSDFPQAWAET
GGMGLAVRQAPLIIPLKATSTPVSIKQYPMSQEARLGIKPHIQRLLDQGILVPCQSPWNT
PLLPVKKPGTNDYRPVQDLREVNKRVEDIHPTVPNPYNLLSGLPPSHQWYTVLDLKDAF
FCLRLHPTSQPLFAFEWRDPEMGISGQLTWTRLPQGFKNSPTLFDEALHRDLADFRIQ
HPDLILLQYVDDLLLAATSELDCQQGTRALLQTLGNLGYRASAKKAQICQKQVKYLGYL
LKEGQRWLTEARKETVMGQPTPKTPRQLREFLGTAGFCRLWIPGFAEMAAPLYPLTKT
GTLFNWGPDQQKAYQEIKQALLTAPALGLPDLTKPFELFVDEKQGYAKGVLTQKLGPW
RRPVAYLSKKLDPVAAGWPPCLRMVAAIAVLTKDAGKLTMGQPLVILAPHAVEALVKQP
PDRWLSNARMTHYQALLLDTDRVQFGPVVALNPATLLPLPEEGLQHNCLDILAEAHGTR
PDLTDQPLPDADHTWYTDGSSLLQEGQRKAGAAVTTETEVIWAKALPAGTSAQRAELIA
LTQALKMAEGKKLNVYTDSRYAFATAHIHGEIYRRRGLLTSEGKEIKNKDEILALLKALFL
PKRLSIIHCPGHQKGHSAEARGNRMADQAARKAAITETPDTSTLLIENSSPSGGSKRTA
DGSEFEPKKKRKV(SEQ ID NO: 389)
KEY:
NLS (SEQ ID NO: 29, 155)
CAS9 (SEQ ID NO: 390)
LINKER 1 (SEQ ID NO: 11)
LINKER 2 (SEQ ID NO: 8)
NPUC INTEIN (SEQ ID NO: 385)
RT (SEQ ID NO: 32)

In various embodiments, using SpCas9 nickase (SEQ ID NO: 37, 1368 amino acids) as an example, the split can between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 37. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20, 20/21, 21/22, 22/23, 23/24, 24/25, 25/26, 26/27, 27/28, 28/29, 29/30, 30/31, 31/32, 32/33, 33/34, 34/35, 35/36, 36/37, 38/39, 39/40, 41/42, 42/43, 43/44, 44/45, 45/46, 46/47, 47/48, 48/49, 49/50, 51/52, 52/53, 53/54, 54/55, 55/56, 56/57, 57/58, 58/59, 59/60, 61/62, 62/63, 63/64, 64/65, 65/66, 66/67, 67/68, 68/69, 69/70, 71/72, 72/73, 73/74, 74/75, 75/76, 76/77, 77/78, 78/79, 79/80, 81/82, 82/83, 83/84, 84/85, 85/86, 86/87, 87/88, 88/89, 89/90, or between any two pairs of adjacent residues between 90-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, and 1350-1368, relative to SpCas9 of SEQ ID NO: 37, at between any two corresponding residues in an amino acid sequence having at least 80%, 85%, 90%, 95%, 98%, 99%, or 99.9% sequence identity with SEQ ID NO: 37, or between any two corresponding residues in a variant or equivalent of SpCas9 of any of amino acid sequences SEQ ID NOs. 31, 37-38, 40, 42, 44-99, or an amino acid sequence having at least 80%, 85%, 90%, 95%, 98%, 99%, or 99.9% sequence identity with any of SEQ ID NOs: 31, 37-38, 40, 42, 44-99.

In various embodiments, the split intein sequences can be engineered by from the intein sequences represented by SEQ ID NOs: 16-23.

In various other embodiments, the split intein sequences can be used as follows:

| INTEIN-N | INTEIN-C |
|---|---|
| NPU-N | NPU-C |
| CLSYETEILTVEYGLLPIGK | IKIATRKYLGKQNVYDIG |
| IVEKRIECTVYSVDNNGNIY | VERDHNFALKNGFIASN |
| TQPVAQWHDRGEQEVFEYCL | (SEQ ID NO: 385) |
| EDGSLIRATKDHKFMTVDGQ | |
| MLPIDEIFERELDLMRVDNL | |
| PNSGGS | |
| (SEQ ID NO: 382) | |

In various embodiments, the split inteins can be used to separately deliver separate portions of a complete Prime editor to a cell, which upon expression in a cell, become reconstituted as a complete Prime editor through the trans splicing.

In some embodiments, the disclosure provides a method of delivering a Prime editor to a cell, comprising:
(a) constructing a first expression vector encoding an N-terminal fragment of the Prime editor fused to a first split intein sequence;
(b) constructing a second expression vector encoding a C-terminal fragment of the Prime editor fused to a second split intein sequence;
(c) delivering the first and second expression vectors to a cell,
   wherein the N-terminal and C-terminal fragment are reconstituted as the Prime editor in the cell as a result of trans splicing activity causing self-excision of the first and second split intein sequences.

The split site in some embodiments can be anywhere in the prime editor fusion, including the napDNAbp domain, the linker, or the reverse transcriptase domain.

In other embodiments, the split site is in the napDNAbp domain.

In still other embodiments, the split site is in the reverse transcriptase or polymerase domain.

In yet other embodiments, the split site is in the linker.

In various embodiments, the present disclosure provides prime editors comprising a napDNAbp (e.g., a Cas9 domain) and a reverse transcriptase wherein one or both of the napDNAbp and/or the reverse transcriptase comprise an intein, for example, a ligand-dependent intein. Typically the intein is a ligand-dependent intein which exhibits no or minimal protein splicing activity in the absence of ligand (e.g., small molecules such as 4-hydroxytamoxifen, peptides, proteins, polynucleotides, amino acids, and nucleotides). Ligand-dependent inteins are known, and include those described in U.S. patent application U.S. Ser. No. 14/004,280, published as U.S. 2014/0065711 A1, the entire contents of which are incorporated herein by reference. In addition, use of split-Cas9 architecture In some embodiments, the intein comprises an amino acid sequence selected from the group consisting of SEQ ID NOs: 16-23, 382, 385, 388.

In various embodiments, the napDNAbp domains are smaller-sized napDNAbp domains as compared to the canonical SpCas9 domain of SEQ ID NO: 37.

The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In one embodiment, as depicted in Example 20, the specification embraces the following split-intein PE constructs, which are split between residues 1024 and 1025 of the canonical SpCas9 (SEQ ID NO: 37) (or which may be referred to as residues 1023 and 1024, respectively, relative to a Met-minus SEQ ID NO: 37).

First, the amino acid sequence of SEQ ID NO: 37 is shown as follows, indicating the location of the split site between 1024 (“K”) and 1025 (“S”) residues:

| Descrip- | | SEQ ID |
|---|---|---|
| tion | Sequence | NO: |
| SpCas9 | MDKKYSIGLDIGTNSVGWAV | SEQ ID |
| Strepto- | ITDEYKVPSKKFKVLGNTDR | NO: 37, |
| coccus | HSIKKNLIGALLFDSGETAE | indi- |
| pyogenes | ATRLKRTARRRYTRRKNRIC | cated |
| M1 | YLQEIFSNEMAKVDDSFFHR | with |
| Swiss- | LEESFLVEEDKKHERHPIFG | split |
| Prot | NIVDEVAYHEKYPTIYHLRK | site |
| Acces- | KLVDSTDKADLRLIYLALAH | 1024/ |
| sion | MIKFRGHFLIEGDLNPDNSD | 1025 |
| No. | VDKLFIQLVQTYNQLFEENP | in |
| Q99ZW2 | INASGVDAKAILSARLSKSR | bold |
| Wild | RLENLIAQLPGEKKNGLFGN | The M |
| type | LIALSLGLTPNFKSNFDLAE | at |
| | DAKLQLSKDTYDDDLDNLLA | posi- |
| | QIGDQYADLFLAAKNLSDAI | tion 1 |
| | LLSDILRVNTEITKAPLSAS | is not |
| | MIKRYDEHHQDLTLLKALVR | neces- |
| | QQLPEKYKEIFFDQSKNGYA | sarily |
| | GYIDGGASQEEFYKFIKPIL | present |
| | EKMDGTEELLVKLNREDLLR | in the |
| | KQRTFDNGSIPHQIHLGELH | Prime |
| | AILRRQEDFYPFLKDNREKI | editor |
| | EKILTFRIPYYVGPLARGNS | in |
| | RFAWMTRKSEETITPWNFEE | certain |
| | VVDKGASAQSFIERMTNFDK | embod- |
| | NLPNEKVLPKHSLLYEYFTV | iments. |
| | YNELTKVKYVTEGMRKPAFL | Thus, |
| | SGEQKKAIVDLLFKTNRKVT | the |
| | VKQLKEDYFKKIECFDSVEI | number- |
| | SGVEDRFNASLGTYHDLLKI | ing of |
| | IKDKDFLDNEENEDILEDIV | the |
| | LTLTLFEDREMIEERLKTYA | split |
| | HLFDDKVMKQLKRRRYTGWG | site is |
| | RLSRKLINGIRDKQSGKTIL | 1023/ |
| | DFLKSDGFANRNFMQLIHDD | 1024 |
| | SLTFKEDIQKAQVSGQGDSL | in the |
| | HEHIANLAGSPAIKKGILQT | case |
| | VKVVDELVKVMGRHKPENIV | that |
| | IEMARENQTTQKGQKNSRER | the |
| | MKRIEEGIKELGSQILKEHP | amino |
| | VENTQLQNEKLYLYYLQNGR | acid |
| | DMYVDQELDINRLSDYDVDH | se- |
| | IVPQSFLKDDSIDNKVLTRS | quence |
| | DKNRGKSDNVPSEEVVKKMK | ex- |
| | NYWRQLLNAKLITQRKFDNL | cludes |
| | TKAERGGLSELDKAGFIKRQ | Met at |
| | LVETRQITKHVAQILDSRMN | posi- |
| | TKYDENDKLIREVKVITLKS | tion 1. |
| | KLVSDFRKDFQFYKVREINN | |
| | YHHAHDAYLNAVVGTALIKK | |
| | YPKLESEFVYGDYKVYDVRK | |
| | MIAKSEQEIGKATAKYFFYS | |
| | NIMNFFKTEITLANGEIRKR | |
| | PLIETNGETGEIVWDKGRDF | |
| | ATVRKVLSMPQVNIVKKTEV | |
| | QTGGFSKESILPKRNSDKLI | |
| | ARKKDWDPKKYGGFDSPTVA | |
| | YSVLVVAKVEKGKSKKLKSV | |
| | KELLGITIMERSSFEKNPID | |
| | FLEAKGYKEVKKDLIIKLPK | |
| | YSLFELENGRKRMLASAGEL | |
| | QKGNELALPSKYVNFLYLAS | |
| | HYEKLKGSPEDNEQKQLFVE | |
| | QHKHYLDEIIEQISEFSKRV | |
| | ILADANLDKVLSAYNKHRDK | |
| | PIREQAENIIHLFTLTNLGA | |
| | PAAFKYFDTTIDRKRYTSTK | |
| | EVLDATLIHQSITGLYETRI | |
| | DLSQLGGD | |

In this configuration, the amino acid sequence of N-terminal half (amino acids 1-1024) is as follows:

(SEQ ID NO: 391)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAK.

In this configuration, the amino acid sequence of N-terminal half (amino acids 1-1023) (where the protein is Met-minus at position 1) is as follows:

(SEQ ID NO: 392)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAK.

In this configuration, the amino acid sequence of C-terminal half (amino acids 1024-1368 (or counted as amino acids 1023-1367 in a Met-minus Cas9) is as follows:

(SEQ ID NO: 393)
SEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSF
EKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAF
KYFDTTIDRKRYTSTKEVLDATLIHQSITGLYETRIDLSQLGGD.

As shown in Example 20, the PE2 (which is based on SpCas9 of SEQ ID NO: 37) construct was split at position 1023/1024 (relative to a Met-minus SEQ ID NO: 37) into two separate constructs, as follows:

SpPE2 split at 1023/1024 N terminal half
(SEQ ID NO: 394)
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANL
AGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVY

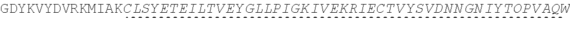

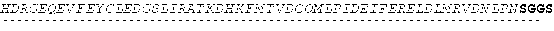

KRTADGSEFEPKKKRKV

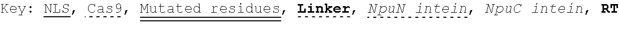

SpPE2 split at 1023/1024 C terminal half

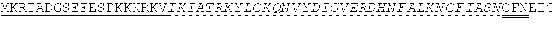

KATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLG

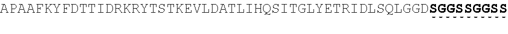

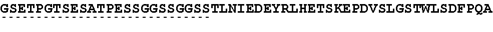

WAETGGMGLAVRQAPLIIPLKATSTPVSIKQYPMSQEARLGIKPHIQRLLDQGIL
VPCQSPWNTPLLPVKKPGTNDYRPVQDLREVNKRVEDIHPTVPNPYNLLSGLPP
SHQWYTVLDLKDAFFCLRLHPTSQPLFAFEWRDPEMGISGQLTWTRLPQGFKN
SPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLDPVAAG
WPPCLRMVAAIAVLTKDAGKLTMGQPLVILAPHAVEALVKQPPDRWLSNARM
THYQALLLDTDRVQFGPVVALNPATLLPLPEEGLQHNCLDILAEAHGTRPDLTD
QPLPDADHTWYTDGSSLLQEGQRKAGAAVTTETEVIWAKALPAGTSAQRAELI
ALTQALKMAEGKKLNVYTDSRYAFATAHIHGEIYRRRGWLTSEGKEIKNKDEI
LALLKALFLPKRLSIIHCPGHQKGHSAEARGNRMADQAARKAAITETPDTSTLLI

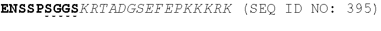

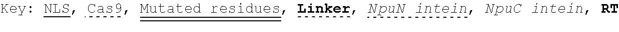

The present disclosure also contemplates methods of delivering split-intein prime editors to cells and/or treating cells with split-intein prime editors.

In some embodiments, the disclosure provides a method of delivering a Prime editor to a cell, comprising:
(a) constructing a first expression vector encoding an N-terminal fragment of the Prime editor fused to a first split intein sequence;
(b) constructing a second expression vector encoding a C-terminal fragment of the Prime editor fused to a second split intein sequence;
(c) delivering the first and second expression vectors to a cell,
   wherein the N-terminal and C-terminal fragment are reconstituted as the Prime editor in the cell as a result of trans splicing activity causing self-excision of the first and second split intein sequences.

In certain embodiments, the N-terminal fragment of the Prime editor fused to a first split intein sequence is SEQ ID NO: 394, or an amino acid sequence having at least 80%, at least 85%, at least 90%, at least 95%, at least 98%, or at least 99.9% sequence identity with SEQ ID NO: 394.

In other embodiments, the C-terminal fragment of the Prime editor fused to a first split intein sequence is SEQ ID NO: 395, or an amino acid sequence having at least 80%, at least 85%, at least 90%, at least 95%, at least 98%, or at least 99.9% sequence identity with SEQ ID NO: 395.

In other embodiments, the disclosure provides a method of editing a target DNA sequence within a cell, comprising:
(a) constructing a first expression vector encoding an N-terminal fragment of the Prime editor fused to a first split intein sequence;
(b) constructing a second expression vector encoding a C-terminal fragment of the Prime editor fused to a second split intein sequence;
(c) delivering the first and second expression vectors to a cell,
   wherein the N-terminal and C-terminal fragment are reconstituted as the Prime editor in the cell as a result of trans splicing activity causing self-excision of the first and second split intein sequences.

In certain embodiments, the N-terminal fragment of the Prime editor fused to a first split intein sequence is SEQ ID NO: 394, or an amino acid sequence having at least 80%, at least 85%, at least 90%, at least 95%, at least 98%, or at least 99.9% sequence identity with SEQ ID NO: 394.

In other embodiments, the C-terminal fragment of the Prime editor fused to a first split intein sequence is SEQ ID NO: 395, or an amino acid sequence having at least 80%, at least 85%, at least 90%, at least 95%, at least 98%, or at least 99.9% sequence identity with SEQ ID NO: 395.

### Delivery of PE Ribonucleoprotein Complexes

In this aspect, the prime editors may be delivered by non-viral delivery strategies involving delivery of a prime editor complexed with a pegRNA (i.e., a PE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional reference may be made to the following references that discuss approaches for non-viral delivery of ribonucleoprotein complexes, each of which are incorporated herein by reference.
Chen, Sean, et al. “Highly efficient mouse genome editing by CRISPR ribonucleoprotein electroporation of zygotes.” Journal of Biological Chemistry (2016): jbc-M116. PubMed
Zuris, John A., et al. “Cationic lipid-mediated delivery of proteins enables efficient protein-based genome editing in vitro and in vivo.” Nature biotechnology 33.1 (2015): 73. PubMed
Rouet, Romain, et al. “Receptor-Mediated Delivery of CRISPR-Cas9 Endonuclease for Cell-Type-Specific Gene Editing.” Journal of the American Chemical Society 140.21 (2018): 6596-6603. PubMed.

FIG. 68C provides data showing that various disclosed PE ribonucleoprotein complexes (PE2 at high concentration, PE3 at high concentration and PE3 at low concentration) can be delivered in this manner.

Delivery of PE by mRNA

Another method that may be employed to deliver prime editors and/or pegRNAs to cells in which prime editing-based genome editing is desired is by employing the use of messenger RNA (mRNA) delivery methods and technologies. Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1, each of which are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27(4): 710-728.

In contrast to DNA vector encoding prime editors, the use of RNA as delivery agent for prime editors has the advantage that the genetic material does not have to enter the nucleus to perform its function. The delivered mRNA may be directly translated in the cytoplasm into the desired protein (e.g., prime editor) and nucleic acid products (e.g., pegRNA). However, in order to be more stable (e.g., resist RNA-degrading enzymes in the cytoplasm), it is in some embodiments necessary to stabilize the mRNA to improve delivery efficiency. Certain delivery carriers such as cationic lipids or polymeric delivery carriers can also help protect the transfected mRNA from endogenous RNase enzymes that might otherwise degrade the therapeutic mRNA encoding the desired prime editor. In addition, despite the increased stability of modified mRNA, delivery of mRNA, particularly mRNA encoding full-length protein, to cells in vivo in a manner that allows therapeutic levels of protein production remains a challenge.

With some exceptions, the intracellular delivery of mRNA is generally more challenging than that of small oligonucleotides, and it requires encapsulation into a delivery nanoparticle, in part due to the significantly larger size of mRNA molecules (300-5,000 kDa, ˜1-15 kb) as compared to other types of RNAs (small interfering RNAs [siRNAs], ˜14 kDa; antisense oligonucleotides [ASOs], 4-10 kDa).

mRNA must cross the cell membrane in order to reach the cytoplasm. The cell membrane is a dynamic and formidable barrier to intracellular delivery. It is made up primarily of a lipid bilayer of zwitterionic and negatively charged phospholipids, where the polar heads of the phospholipids point toward the aqueous environment and the hydrophobic tails form a hydrophobic core.

In some embodiments, the mRNA compositions of the disclosure comprise mRNA (encoding a prime editor and/or pegRNA), a transport vehicle, and optionally an agent that facilitates contact with the target cell and subsequent transfection.

In some embodiments, the mRNA can include one or more modifications that confer stability to the mRNA (e.g., compared to the wild-type or native version of the mRNA) and is involved in the associated abnormal expression of the protein. One or more modifications to the wild type that correct the defect may also be included. For example, the nucleic acids of the invention can include modifications of one or both of a 5′ untranslated region or a 3′ untranslated region. Such modifications may include the inclusion of sequences encoding a partial sequence of the cytomegalovirus (CMV) immediate early 1 (IE1) gene, poly A tail, Cap1 structure, or human growth hormone (hGH). In some embodiments, the mRNA is modified to reduce mRNA immunogenicity.

In one embodiment, the “prime editor” mRNA in the composition of the invention can be formulated in a liposome transfer vehicle to facilitate delivery to target cells. Contemplated transfer vehicles can include one or more cationic lipids, non-cationic lipids, and/or PEG-modified lipids. For example, the transfer vehicle can include at least one of the following cationic lipids: C12-200, DLin-KC2-DMA, DODAP, HGT4003, ICE, HGT5000, or HGT5001. In embodiments, the transfer vehicle comprises cholesterol (chol) and/or PEG modified lipids. In some embodiments, the transfer vehicle comprises DMG-PEG2K. In certain embodiments, the transfer vehicle has the following lipid formulation: C12-200, DOPE, chol, DMG-PEG2K; DODAP, DOPE, cholesterol, DMG-PEG2K; HGT5000, DOPE, chol, DMG-PEG2K, HGT5001, DOPE, chol, one of DMG-PEG2K.

The present disclosure also provides compositions and methods useful for facilitating transfection of target cells with one or more PE-encoding mRNA molecules. For example, the compositions and methods of the present invention contemplate the use of targeting ligands that can increase the affinity of the composition for one or more target cells. In one embodiment, the targeting ligand is apolipoprotein B or apolipoprotein E, and the corresponding target cells express low density lipoprotein receptors and thus promote recognition of the targeting ligand. A vast number of target cells can be preferentially targeted using the methods and compositions of the present disclosure. For example, contemplated target cells include hepatocytes, epithelial cells, hematopoietic cells, epithelial cells, endothelial cells, lung cells, bone cells, stem cells, mesenchymal cells, nerve cells, heart cells, adipocytes, vascular smooth muscle Includes cells, cardiomyocytes, skeletal muscle cells, beta cells, pituitary cells, synovial lining cells, ovarian cells, testis cells, fibroblasts, B cells, T cells, reticulocytes, leukocytes, granulocytes, and tumor cells However, it is not limited to these.

In some embodiments, the PE-encoding mRNA may optionally have chemical or biological modifications which, for example, improve the stability and/or half-life of such mRNA or which improve or otherwise facilitate protein production. Upon transfection, a natural mRNA in the compositions of the invention may decay with a half-life of between 30 minutes and several days. The mRNAs in the compositions of the disclosure may retain at least some ability to be translated, thereby producing a functional protein or enzyme. Accordingly, the invention provides compositions comprising and methods of administering a stabilized mRNA. In some embodiments, the activity of the mRNA is prolonged over an extended period of time. For example, the activity of the mRNA may be prolonged such that the compositions of the present disclosure are administered to a subject on a semi-weekly or bi-weekly basis, or more preferably on a monthly, bi-monthly, quarterly or an annual basis. The extended or prolonged activity of the mRNA of the present invention is directly related to the quantity of protein or enzyme produced from such mRNA. Similarly, the activity of the compositions of the present disclosure may be further extended or prolonged by modifications made to improve or enhance translation of the mRNA. Furthermore, the quantity of functional protein or enzyme produced by the target cell is a function of the quantity of mRNA delivered to the target cells and the stability of such mRNA. To the extent that the stability of the mRNA of the present invention may be improved or enhanced, the half-life, the activity of the produced protein or enzyme and the dosing frequency of the composition may be further extended.

Accordingly, in some embodiments, the mRNA in the compositions of the disclosure comprise at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g. the wild-type version of the mRNA). Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the mRNAs used in the compositions of the disclosure have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

Other suitable polynucleotide modifications that may be incorporated into the PE-encoding mRNA used in the compositions of the disclosure include, but are not limited to, 4′-thio-modified bases: 4′-thio-adenosine, 4′-thio-guanosine, 4′-thio-cytidine, 4′-thio-uridine, 4′-thio-5-methyl-cytidine, 4′-thio-pseudouridine, and 4′-thio-2-thiouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. The term modification also includes, for example, the incorporation of non-nucleotide linkages or modified nucleotides into the mRNA sequences of the present invention (e.g., modifications to one or both of the 3′ and 5′ ends of an mRNA molecule encoding a functional protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

In some embodiments, PE-encoding mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Naturally occurring cap structures comprise a 7-methyl guanosine that is linked via a triphosphate bridge to the 5′-end of the first transcribed nucleotide, resulting in a dinucleotide cap of m7G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

Additional cap analogs include, but are not limited to, a chemical structures selected from the group consisting of m7GpppG, m7GpppA, m7GpppC; unmethylated cap analogs (e.g., GpppG); dimethylated cap analog (e.g., m2,7GpppG), trimethylated cap analog (e.g., m2,2,7GpppG), dimethylated symmetrical cap analogs (e.g., m7Gpppm7G), or anti reverse cap analogs (e.g., ARCA; m7,2′OmeGpppG, m72′dGpppG, m7,3′OmeGpppG, m7,3′dGpppG and their tetraphosphate derivatives) (see, e.g., Jemielity, J. et al., “Novel ‘anti-reverse’ cap analogs with superior translational properties”, RNA, 9: 1108-1122 (2003)).

Typically, the presence of a “tail” serves to protect the mRNA from exonuclease degradation. A poly A or poly U tail is thought to stabilize natural messengers and synthetic sense RNA. Therefore, in certain embodiments a long poly A or poly U tail can be added to an mRNA molecule thus rendering the RNA more stable. Poly A or poly U tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

Typically, the length of a poly A or poly U tail can be at least about 10, 50, 100, 200, 300, 400 at least 500 nucleotides. In some embodiments, a poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A or poly U tail or may substitute the poly-A or poly U tail.

PE-encoding mRNAs according to the present disclosure may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In embodiments involving mRNA delivery, the ratio of the mRNA encoding the Prime editor to the pegRNA may be important for efficient editing. In certain embodiments, the weight ratio of mRNA (encoding the Prime editor) to pegRNA is 1:1. In certain other embodiments, the weight ratio of mRNA (encoding the Prime editor) to pegRNA is 2:1. In still other embodiments, the weight ratio of mRNA (encoding the Prime editor) to pegRNA is 1:2. In still further embodiments, the weight ratio of mRNA (encoding the Prime editor) to pegRNA is selected from the group consisting of about 1:1000, 1:900; 1:800; 1:700; 1:600; 1:500; 1:400; 1:300; 1:200; 1:100; 1:90; 1:80; 1:70; 1:60; 1:50; 1:40; 1:30; 1:20; 1:10; and 1:1. In other embodiments, the weight ratio of mRNA (encoding the Prime editor) to pegRNA is selected from the group consisting of about 1:1000, 1:900; 800:1; 700:1; 600:1; 500:1; 400:1; 300:1; 200:1; 100:1; 90:1; 80:1; 70:1; 60:1; 50:1; 40:1; 30:1; 20:1; 10:1; and 1:1.

### [9] Methods of Treatment

The instant disclosure provides methods for the treatment of a subject diagnosed with a disease associated with or caused by a point mutation, or other mutations (e.g., deletion, insertion, inversion, duplication, etc.) that can be corrected by the prime editing system provided herein, as exemplified, but not limited to prion disease, trinucleotide repeat expansion disease, or CDKL5 Deficiency Disorder (CDD) (e.g., Example 6 herein).

Virtually any disease-causing genetic defect may be repaired by using prime editing, which includes the selection of an appropriate prime editor (including a napDNAbp and a polymerase (e.g., a reverse transcriptase), and designing of an appropriate pegRNA designed to (a) target the appropriate target DNA containing an edit site, and (b) provide a template for the synthesis of a single strand of DNA from the 3′ end of the nick site that includes the desired edit which displaces and replaces the endogenous strand immediately downstream of the nick site. Prime editing can be used, without limitation, to (a) install mutation-correcting changes to a nucleotide sequence, (b) install protein and RNA tags, (c) install immunoepitopes on proteins of interest, (d) install inducible dimerization domains in proteins, (e) install or remove sequences to alter that activity of a biomolecule, (f) install recombinase target sites to direct specific genetic changes, and (g) mutagenesis of a target sequence by using an error-prone RT.

The method of treating a disorder can involve as an early step the design of an appropriate pegRNA and prime editor in accordance with the methods described herein, which include a number of considerations that may be taken into account, such as:
(a) the target sequence, i.e., the nucleotide sequence in which one or more nucleobase modifications are desired to be installed by the prime editor;
(b) the location of the cut site within the target sequence, i.e., the specific nucleobase position at which the prime editor will induce a single-stand nick to create a 3′ end RT primer sequence on one side of the nick and the 5′ end endogenous flap on the other side of the nick (which ultimately is removed by FEN1 or equivalent thereto and replaced by the 3′ ssDNA flap. The cut site creates the 3′ end primer sequence which becomes extended by the polymerase of the Prime editor (e.g., a RT enzyme) during RNA-dependent DNA polymerization to create the 3′ ssDNA flap containing the desired edit, which then replaces the 5′ endogenous DNA flap in the target sequence.
(c) the available PAM sequences (including the canonical SpCas9 PAM sites, as well as non-canonical PAM sites recognized by Cas9 variants and equivalents with expanded or differing PAM specificities);
(d) the spacing between the available PAM sequences and the location of the cut site in the PAM strand;
(e) the particular Cas9, Cas9 variant, or Cas9 equivalent of the prime editor available to be used (which in part is dictated by the available PAM);
(f) the sequence and length of the primer binding site;
(g) the sequence and length of the edit template;
(h) the sequence and length of the homology arm;
(i) the spacer sequence and length; and
(j) the gRNA core sequence.

A suitable pegRNA, and optionally a nicking-sgRNA design guide for second-site nicking, can be designed by way of the following exemplary step-by-step set of instructions which takes into account one or more of the above considerations. The steps reference the examples shown in FIGS. 70A-70I.
1. Define the target sequence and the edit. Retrieve the sequence of the target DNA region (˜200 bp) centered around the location of the desired edit (point mutation, insertion, deletion, or combination thereof). See FIG. 70A.
2. Locate target PAMs. Identify PAMs in the proximity to the desired edit location. PAMs can be identified on either strand of DNA proximal to the desired edit location. While PAMs close to the edit position are preferred (i.e., wherein the nick site is less than 30 nt from the edit position, or less than 29 nt, 28 nt, 27 nt, 26 nt, 25 nt, 24 nt, 23 nt, 22 nt, 21 nt, 20 nt, 19 nt, 18 nt, 17 nt, 16 nt, 15 nt, 14 nt, 13 nt, 12 nt, 11 nt, 10 nt, 9 nt, 8 nt, 7 nt, 6 nt, 5 nt, 4 nt, 3 nt, or 2 nt from the edit position to the nick site), it is possible to install edits using protospacers and PAMs that place the nick ≥30 nt from the edit position. See FIG. 70B.
3. Locate the nick sites. For each PAM being considered, identify the corresponding nick site and on which strand. For Sp Cas9 H840A nickase, cleavage occurs in the PAM-containing strand between the 3^{rd} and 4^{th} bases 5′ to the NGG PAM. All edited nucleotides must exist 3′ of the nick site, so appropriate PAMs must place the nick 5′ to the target edit on the PAM-containing strand. In the example shown below, there are two possible PAMs. For simplicity, the remaining steps will demonstrate the design of a pegRNA using PAM 1 only. See FIG. 70C.
4. Design the spacer sequence. The protospacer of SpCas9 corresponds to the 20 nucleotides 5′ to the NGG PAM on the PAM-containing strand. Efficient Pol III transcription initiation requires a G to be the first transcribed nucleotide. If the first nucleotide of the protospacer is a G, the spacer sequence for the pegRNA is simply the protospacer sequence. If the first nucleotide of the protospacer is not a G, the spacer sequence of the pegRNA is G followed by the protospacer sequence. See FIG. 70D.
5. Design a primer binding site (PBS). Using the starting allele sequence, identify the DNA primer on the PAM-containing strand. The 3′ end of the DNA primer is the nucleotide just upstream of the nick site (i.e. the 4^{th} base 5′ to the NGG PAM for Sp Cas9). As a general design principle for use with PE2 and PE3, a pegRNA primer binding site (PBS) containing 12 to 13 nucleotides of complementarity to the DNA primer can be used for sequences that contain ˜40-60% GC content. For sequences with low GC content, longer (14- to 15-nt) PBSs should be tested. For sequences with higher GC content, shorter (8- to 11-nt) PBSs should be tested. Optimal PBS sequences should be determined empirically, regardless of GC content. To design a length-p PBS sequence, take the reverse complement of the first p nucleotides 5′ of the nick site in the PAM-containing strand using the starting allele sequence. See FIG. 70E.
6. Design an RT template (or DNA synthesis template). The RT template (or DNA synthesis template where the polymerase is not reverse transcriptase) encodes the designed edit and homology to the sequence adjacent to the edit. In one embodiment, these regions correspond to the DNA synthesis template of FIG. 3D and FIG. 3E, wherein the DNA synthesis template comprises the “edit template” and the “homology arm.” Optimal RT template lengths vary based on the target site. For short-range edits (positions +1 to +6), it is recommended to test a short (9 to 12 nt), a medium (13 to 16 nt), and a long (17 to 20 nt) RT template. For long-range edits (positions +7 and beyond), it is recommended to use RT templates that extend at least 5 nt (preferably 10 or more nt) past the position of the edit to allow for sufficient 3′ DNA flap homology. For long-range edits, several RT templates should be screened to identify functional designs. For larger insertions and deletions (≥5 nt), incorporation of greater 3′ homology (˜20 nt or more) into the RT template is recommended. Editing efficiency is typically impaired when the RT template encodes the synthesis of a G as the last nucleotide in the reverse transcribed DNA product (corresponding to a C in the RT template of the pegRNA). As many RT templates support efficient prime editing, avoidance of G as the final synthesized nucleotide is recommended when designing RT templates. To design a length-r RT template sequence, use the desired allele sequence and take the reverse complement of the first r nucleotides 3′ of the nick site in the strand that originally contained the PAM. Note that compared to SNP edits, insertion or deletion edits using RT templates of the same length will not contain identical homology. See FIG. 70F.
7. Assemble the full pegRNA sequence. Concatenate the pegRNA components in the following order (5′ to 3′): spacer, scaffold, RT template and PBS. See FIG. 70G.
8. Designing nicking-sgRNAs for PE3. Identify PAMs on the non-edited strand upstream and downstream of the edit. Optimal nicking positions are highly locus-dependent and should be determined empirically. In general, nicks placed 40 to 90 nucleotides 5′ to the position across from the pegRNA-induced nick lead to higher editing yields and fewer indels. A nicking sgRNA has a spacer sequence that matches the 20-nt protospacer in the starting allele, with the addition of a 5′-G if the protospacer does not begin with a G. See FIG. 70H.
9. Designing PE3b nicking-sgRNAs. If a PAM exists in the complementary strand and its corresponding protospacer overlaps with the sequence targeted for editing, this edit could be a candidate for the PE3b system. In the PE3b system, the spacer sequence of the nicking-sgRNA matches the sequence of the desired edited allele, but not the starting allele. The PE3b system operates efficiently when the edited nucleotide(s) falls within the seed region (˜10 nt adjacent to the PAM) of the nicking-sgRNA protospacer. This prevents nicking of the complementary strand until after installation of the edited strand, preventing competition between the pegRNA and the sgRNA for binding the target DNA. PE3b also avoids the generation of simultaneous nicks on both strands, thus reducing indel formation significantly while maintaining high editing efficiency. PE3b sgRNAs should have a spacer sequence that matches the 20-nt protospacer in the desired allele, with the addition of a 5′ G if needed. See FIG. 70I.

The above step-by-step process for designing a suitable pegRNA and a second-site nicking sgRNA is not meant to be limiting in any way. The disclosure contemplates variations of the above-described step-by-step process which would be derivable therefrom by a person of ordinary skill in the art.

Once a suitable pegRNA and Prime editor are selected/designed, they may be administered by a suitable methodology, such as by vector-based transfection (in which one or more vectors comprising DNA encoding the pegRNA and the Prime editor and which are expressed within a cell upon transfection with the vectors), direct delivery of the Prime editor complexed with the pegRNA (e.g., RNP delivery) in a delivery format (e.g., lipid particles, nanoparticles), or by a mRNA-based delivery system. Such methods are described herein in the present disclosure and any know method may be utilized.

The pegRNA and Prime editor (or together, referred to as the PE complex) can be delivered to a cell in a therapeutically effective amount such that upon contacting the target DNA of interest, the desired edit becomes installed therein.

Any disease is conceivably treatable by such methods so long as delivery to the appropriate cells is feasible. The person having ordinary skill in the art will be able to choose and/or select a PE delivery methodology to suit the intended purpose and the intended target cells.

For example, in some embodiments, a method is provided that comprises administering to a subject having such a disease, e.g., a cancer associated with a point mutation as described above, an effective amount of the prime editing system described herein that corrects the point mutation or introduces a deactivating mutation into a disease-associated gene as mediated by homology-directed repair in the presence of a donor DNA molecule comprising desired genetic change. In some embodiments, a method is provided that comprises administering to a subject having such a disease, e.g., a cancer associated with a point mutation as described above, an effective amount of the prime editing system described herein that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

In another aspect, a method is provided that uses a prime editor (e.g., PE1, PE2, or PE3) in combination with a guide RNAs (pegRNAs) to carry out prime editing to directly install or correct mutations in the CDKL5 gene which cause CDKL5 deficiency disorder. In various embodiments, the disclosure provides a complex comprising a prime editor (e.g., PE1, PE2, or PE3) and a pegRNA that is capable of directly installing or correcting more than one mutation in the CDKL5 gene in multiple subjects.

The instant disclosure provides methods for the treatment of additional diseases or disorders, e.g., diseases or disorders that are associated or caused by a point mutation that can be corrected by prime editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1, 9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome; Cohen syndrome; Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional cl inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, A11, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB and IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3, 7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6,and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria; Methylmalonic aciduria cblB type; Methylmalonic aciduria due to methylmalonyl-CoA mutase deficiency; METHYLMALONIC ACIDURIA, mut(0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-H/S, MPS-II, MPS-III-A, MPS-III-B, MPS-III-C, MPS-IV-A, MPS-IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP guanine oxidase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1, 10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40,and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1(nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, lb; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

The target nucleotide sequence may comprise a target sequence (e.g., a point mutation) associated with a disease, disorder, or condition. The target sequence may comprise a T to C (or A to G) point mutation associated with a disease, disorder, or condition, and wherein the deamination of the mutant C base results in mismatch repair-mediated correction to a sequence that is not associated with a disease, disorder, or condition. The target sequence may comprise a G to A (or C to T) point mutation associated with a disease, disorder, or condition, and wherein the deamination of the mutant A base results in mismatch repair-mediated correction to a sequence that is not associated with a disease, disorder, or condition. The target sequence may encode a protein, and where the point mutation is in a codon and results in a change in the amino acid encoded by the mutant codon as compared to a wild-type codon. The target sequence may also be at a splice site, and the point mutation results in a change in the splicing of an mRNA transcript as compared to a wild-type transcript. In addition, the target may be at a non-coding sequence of a gene, such as a promoter, and the point mutation results in increased or decreased expression of the gene.

Thus, in some aspects, the deamination of a mutant C results in a change of the amino acid encoded by the mutant codon, which in some cases can result in the expression of a wild-type amino acid. In other aspects, the deamination of a mutant A results in a change of the amino acid encoded by the mutant codon, which in some cases can result in the expression of a wild-type amino acid.

The methods described herein involving contacting a cell with a composition or rAAV particle can occur in vitro, ex vivo, or in vivo. In certain embodiments, the step of contacting occurs in a subject. In certain embodiments, the subject has been diagnosed with a disease, disorder, or condition.

In some embodiments, the methods disclosed herein involve contacting a mammalian cell with a composition or rAAV particle. In particular embodiments, the methods involve contacting a retinal cell, cortical cell or cerebellar cell.

The split Cas9 protein or split prime editor delivered using the methods described herein preferably have comparable activity compared to the original Cas9 protein or prime editor (i.e., unsplit protein delivered to a cell or expressed in a cell as a whole). For example, the split Cas9 protein or split prime editor retains at least 50% (e.g., at least 50%, at least 60%, at least 70%, at least 80%, at least 90%, at least 95%, at least 98%, at least 99%, or 100%) of the activity of the original Cas9 protein or prime editor. In some embodiments, the split Cas9 protein or split prime editor is more active (e.g., 2-fold, 5-fold, 10-fold, 100-fold, 1000-fold, or more) than that of an original Cas9 protein or prime editor.

The compositions described herein may be administered to a subject in need thereof in a therapeutically effective amount to treat and/or prevent a disease or disorder the subject is suffering from. Any disease or disorder that maybe treated and/or prevented using CRISPR/Cas9-based genome-editing technology may be treated by the split Cas9 protein or the split prime editor described herein. It is to be understood that, if the nucleotide sequences encoding the split Cas9 protein or the prime editor does not further encode a gRNA, a separate nucleic acid vector encoding the gRNA may be administered together with the compositions described herein.

Exemplary suitable diseases, disorders or conditions include, without limitation the disease or disorder is selected from the group consisting of: cystic fibrosis, phenylketonuria, epidermolytic hyperkeratosis (EHK), chronic obstructive pulmonary disease (COPD), Charcot-Marie-Toot disease type 4J, neuroblastoma (NB), von Willebrand disease (vWD), myotonia congenital, hereditary renal amyloidosis, dilated cardiomyopathy, hereditary lymphedema, familial Alzheimer's disease, prion disease, chronic infantile neurologic cutaneous articular syndrome (CINCA), congenital deafness, Niemann-Pick disease type C (NPC) disease, and desmin-related myopathy (DRM). In particular embodiments, the disease or condition is Niemann-Pick disease type C (NPC) disease.

In some embodiments, the disease, disorder or condition is associated with a point mutation in an NPC gene, a DNMT1 gene, a PCSK9 gene, or a TMC1 gene. In certain embodiments, the point mutation is a T3182C mutation in NPC, which results in an I1061T amino acid substitution.

In certain embodiments, the point mutation is an A545G mutation in TMC1, which results in a Y182C amino acid substitution. TMC1 encodes a protein that forms mechanosensitive ion channels in sensory hair cells of the inner ear and is required for normal auditory function. The Y182C amino acid substitution is associated with congenital deafness.

In some embodiments, the disease, disorder or condition is associated with a point mutation that generates a stop codon, for example, a premature stop codon within the coding region of a gene.

Additional exemplary diseases, disorders and conditions include cystic fibrosis (see, e.g., Schwank et al., Functional repair of CFTR by CRISPR/Cas9 in intestinal stem cell organoids of cystic fibrosis patients. Cell stem cell. 2013; 13: 653-658; and Wu et. al., Correction of a genetic disease in mouse via use of CRISPR-Cas9. Cell stem cell. 2013; 13: 659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97: 312-320, and Ali et al., Hematol. 2014; 93: 381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70: 821-828, see also accession number P04264 in the UNIPROT database at www[dot]uniprot[dot]org; chronic obstructive pulmonary disease (COPD)—e.g., leucine to proline mutation at position 54 or 55 (if counting the initiator methionine) or a homologous residue in the processed form of ai-antitrypsin or residue 78 in the unprocessed form or a homologous residue (T>C mutation)—see, e.g., Poller et al., Genomics. 1993; 17: 740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLoS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82: 66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590: 3449-3464; hereditary renal amyloidosis—e.g., stop codon to arginine mutation at position 78 or a homologous residue in the processed form of apolipoprotein A11 or at position 101 or a homologous residue in the unprocessed form (T>C mutation)—see, e.g., Yazaki et al., Kidney Int. 2003; 64: 11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19: 369-372; hereditary lymphedema—e.g., histidine to arginine mutation at position 1035 or a homologous residue in VEGFR3 tyrosine kinase (A>G mutation), see, e.g., Irrthum et al., Am. J. Hum. Genet. 2000; 67: 295-301; familial Alzheimer's disease—e.g., isoleucine to valine mutation at position 143 or a homologous residue in presenilin1 (A>G mutation), see, e.g., Gallo et. al., J. Alzheimer's disease. 2011; 25: 425-431; Prion disease—e.g., methionine to valine mutation at position 129 or a homologous residue in prion protein (A>G mutation)—see, e.g., Lewis et. al., J. of General Virology. 2006; 87: 2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109: 2903-2911; and desmin-related myopathy (DRM)—e.g., arginine to glycine mutation at position 120 or a homologous residue in αβ crystallin (A>G mutation)—see, e.g., Kumar et al., J. Biol. Chem. 1999; 274: 24137-24141. The entire contents of all references and database entries is incorporated herein by reference.

### Trinucleotide Repeat Expansion Disease

Trinucleotide repeat expansion is associated with a number of human diseases, including Huntington's Disease, Fragile X syndrome, and Friedreich's ataxia. The most common trinucleotide repeat contains CAG triplets, though GAA triplets (Friedreich's ataxia) and CGG triplets (Fragile X syndrome) also occur. Inheriting a predisposition to expansion, or acquiring an already expanded parental allele, increases the likelihood of acquiring the disease. Pathogenic expansions of trinucleotide repeats could hypothetically be corrected using prime editing.

A region upstream of the repeat region can be nicked by an RNA-guided nuclease, then used to prime synthesis of a new DNA strand that contains a healthy number of repeats (which depends on the particular gene and disease), in accordance with the general mechanism outlined in FIG. 1G or FIG. 22. After the repeat sequence, a short stretch of homology is added that matches the identity of the sequence adjacent to the other end of the repeat (red strand). Invasion of the newly synthesized strand by the prime editor, and subsequent replacement of the endogenous DNA with the newly synthesized flap, leads to a contracted repeat allele. The term “contracted” refers to a shortening of the length of the nucleotide repeat region, thereby resulting in repairing the trinucleotide repeat region.

The prime editing system or prime editing (PE) system described herein may be used to contract trinucleotide repeat mutations (or “triplet expansion diseases”) to treating conditions such as Huntington's disease and other trinucleotide repeat disorders. Trinucleotide repeat expansion disorders are complex, progressive disorders that involve developmental neurobiology and often affect cognition as well as sensori-motor functions. The disorders show genetic anticipation (i.e. increased severity with each generation). The DNA expansions or contractions usually happen meiotically (i.e. during the time of gametogenesis, or early in embryonic development), and often have sex-bias meaning that some genes expand only when inherited through the female, others only through the male. In humans, trinucleotide repeat expansion disorders can cause gene silencing at either the transcriptional or translational level, which essentially knocks out gene function. Alternatively, trinucleotide repeat expansion disorders can cause altered proteins generated with large repetitive amino acid sequences that either abrogate or change protein function, often in a dominant-negative manner (e.g. poly-glutamine diseases).

Without wishing to be bound by theory, triplet expansion is caused by slippage during DNA replication or during DNA repair synthesis. Because the tandem repeats have identical sequence to one another, base pairing between two DNA strands can take place at multiple points along the sequence. This may lead to the formation of “loop out” structures during DNA replication or DNA repair synthesis. This may lead to repeated copying of the repeated sequence, expanding the number of repeats. Additional mechanisms involving hybrid RNA:DNA intermediates have been proposed. Prime editing may be used to reduce or eliminate these triplet expansion regions by deletion one or more or the offending repeat codon triplets. In an embodiment of this use, FIG. 23, provides a schematic of a pegRNA design for contracting or reducing trinucleotide repeat sequences with prime editing.

Prime editing may be implemented to contract triplet expansion regions by nicking a region upstream of the triplet repeat region with the prime editor comprising a pegRNA appropriated targeted to the cut site. The prime editor then synthesizes a new DNA strand (ssDNA flap) based on the pegRNA as a template (i.e., the edit template thereof) that codes for a healthy number of triplet repeats (which depends on the particular gene and disease). The newly synthesized ssDNA strand comprising the healthy triplet repeat sequence also is synthesized to include a short stretch of homology (i.e., the homology arm) that matches the sequence adjacent to the other end of the repeat (red strand). Invasion of the newly synthesized strand, and subsequent replacement of the endogenous DNA with the newly synthesized ssDNA flap, leads to a contracted repeat allele.

Depending on the particular trinucleotide expansion disorder, the defect-inducing triplet expansions may occur in “trinucleotide repeat expansion proteins.” Trinucleotide repeat expansion proteins are a diverse set of proteins associated with susceptibility for developing a trinucleotide repeat expansion disorder, the presence of a trinucleotide repeat expansion disorder, the severity of a trinucleotide repeat expansion disorder or any combination thereof. Trinucleotide repeat expansion disorders are divided into two categories determined by the type of repeat. The most common repeat is the triplet CAG, which, when present in the coding region of a gene, codes for the amino acid glutamine (Q). Therefore, these disorders are referred to as the polyglutamine (polyQ) disorders and comprise the following diseases: Huntington Disease (HD); Spinobulbar Muscular Atrophy (SBMA); Spinocerebellar Ataxias (SCA types 1, 2, 3, 6, 7, and 17); and Dentatorubro-Pallidoluysian Atrophy (DRPLA). The remaining trinucleotide repeat expansion disorders either do not involve the CAG triplet or the CAG triplet is not in the coding region of the gene and are, therefore, referred to as the non-polyglutamine disorders. The non-polyglutamine disorders comprise Fragile X Syndrome (FRAXA); Fragile XE Mental Retardation (FRAXE); Friedreich Ataxia (FRDA); Myotonic Dystrophy (DM); and Spinocerebellar Ataxias (SCA types 8, and 12).

The proteins associated with trinucleotide repeat expansion disorders can be selected based on an experimental association of the protein associated with a trinucleotide repeat expansion disorder to a trinucleotide repeat expansion disorder. For example, the production rate or circulating concentration of a protein associated with a trinucleotide repeat expansion disorder may be elevated or depressed in a population having a trinucleotide repeat expansion disorder relative to a population lacking the trinucleotide repeat expansion disorder. Differences in protein levels may be assessed using proteomic techniques including but not limited to Western blot, immunohistochemical staining, enzyme linked immunosorbent assay (ELISA), and mass spectrometry. Alternatively, the proteins associated with trinucleotide repeat expansion disorders may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Non-limiting examples of proteins associated with trinucleotide repeat expansion disorders which can be corrected by prime editing include AR (androgen receptor), FMR1 (fragile X mental retardation 1), HTT (huntingtin), DMPK (dystrophia myotonica-protein kinase), FXN (frataxin), ATXN2 (ataxin 2), ATN1 (atrophin 1), FEN1 (flap structure-specific endonuclease 1), TNRC6A (trinucleotide repeat containing 6A), PABPN1 (poly(A) binding protein, nuclear 1), JPH3 (junctophilin 3), MED15 (mediator complex subunit 15), ATXN1 (ataxin 1), ATXN3 (ataxin 3), TBP (TATA box binding protein), CACNA1A (calcium channel, voltage-dependent, P/Q type, alpha 1A subunit), ATXN80S (ATXN8 opposite strand (non-protein coding)), PPP2R2B (protein phosphatase 2, regulatory subunit B, beta), ATXN7 (ataxin 7), TNRC6B (trinucleotide repeat containing 6B), TNRC6C (trinucleotide repeat containing 6C), CELF3 (CUGBP, Elav-like family member 3), MAB21L1 (mab-21-like 1 (C. elegans)), MSH2 (mutS homolog 2, colon cancer, nonpolyposis type 1 (E. coli)), TMEM185A (transmembrane protein 185A), SIX5 (SIX homeobox 5), CNPY3 (canopy 3 homolog (zebrafish)), FRAXE (fragile site, folic acid type, rare, fra(X)(q28) E), GNB2 (guanine nucleotide binding protein (G protein), beta polypeptide 2), RPL14 (ribosomal protein L14), ATXN8 (ataxin 8), INSR (insulin receptor), TTR (transthyretin), EP400 (E1A binding protein p400), GIGYF2 (GRB10 interacting GYF protein 2), OGG1 (8-oxoguanine DNA glycosylase), STC1 (stanniocalcin 1), CNDP1 (carnosine dipeptidase 1 (metallopeptidase M20 family)), C10orf2 (chromosome 10 open reading frame 2), MAML3 mastermind-like 3 (Drosophila), DKC1 (dyskeratosis congenita 1, dyskerin), PAXIP1 (PAX interacting (with transcription-activation domain) protein 1), CASK (calcium/calmodulin-dependent serine protein kinase (MAGUK family)), MAPT (microtubule-associated protein tau), SP1 (Sp1 transcription factor), POLG (polymerase (DNA directed), gamma), AFF2 (AF4/FMR2 family, member 2), THBS1 (thrombospondin 1), TP53 (tumor protein p53), ESR1 (estrogen receptor 1), CGGBP1 (CGG triplet repeat binding protein 1), ABT1 (activator of basal transcription 1), KLK3 (kallikrein-related peptidase 3), PRNP (prion protein), JUN (jun oncogene), KCNN3 (potassium intermediate/small conductance calcium-activated channel, subfamily N, member 3), BAX (BCL2-associated X protein), FRAXA (fragile site, folic acid type, rare, fra(X)(q27.3) A (macroorchidism, mental retardation)), KBTBD10 (kelch repeat and BTB (POZ) domain containing 10), MBNL1 (muscleblind-like (Drosophila)), RAD51 (RAD51 homolog (RecA homolog, E. coli) (S. cerevisiae)), NCOA3 (nuclear receptor coactivator 3), ERDAl (expanded repeat domain, CAG/CTG 1), TSC1 (tuberous sclerosis 1), COMP (cartilage oligomeric matrix protein), GCLC (glutamate-cysteine ligase, catalytic subunit), RRAD (Ras-related associated with diabetes), MSH3 (mutS homolog 3 (E. coli)), DRD2 (dopamine receptor D2), CD44 (CD44 molecule (Indian blood group)), CTCF (CCCTC-binding factor (zinc finger protein)), CCND1 (cyclin D1), CLSPN (claspin homolog (Xenopus laevis)), MEF2A (myocyte enhancer factor 2A), PTPRU (protein tyrosine phosphatase, receptor type, U), GAPDH (glyceraldehyde-3-phosphate dehydrogenase), TRIM22 (tripartite motif-containing 22), WT1 (Wilms tumor 1), AHR (aryl hydrocarbon receptor), GPX1 (glutathione peroxidase 1), TPMT (thiopurine S-methyltransferase), NDP (Norrie disease (pseudoglioma)), ARX (aristaless related homeobox), MUS81 (MUS81 endonuclease homolog (S. cerevisiae)), TYR (tyrosinase (oculocutaneous albinism IA)), EGR1 (early growth response 1), UNG (uracil-DNA glycosylase), NUMBL (numb homolog (Drosophila)-like), FABP2 (fatty acid binding protein 2, intestinal), EN2 (engrailed homeobox 2), CRYGC (crystallin, gamma C), SRP14 (signal recognition particle 14 kDa (homologous Alu RNA binding protein)), CRYGB (crystallin, gamma B), PDCD1 (programmed cell death 1), HOXA1 (homeobox A1), ATXN2L (ataxin 2-like), PMS2 (PMS2 postmeiotic segregation increased 2 (S. cerevisiae)), GLA (galactosidase, alpha), CBL (Cas-Br-M (murine) ecotropic retroviral transforming sequence), FTH1 (ferritin, heavy polypeptide 1), IL12RB2 (interleukin 12 receptor, beta 2), OTX2 (orthodenticle homeobox 2), HOXA5 (homeobox A5), POLG2 (polymerase (DNA directed), gamma 2, accessory subunit), DLX2 (distal-less homeobox 2), SIRPA (signal-regulatory protein alpha), OTX1 (orthodenticle homeobox 1), AHRR (aryl-hydrocarbon receptor repressor), MANF (mesencephalic astrocyte-derived neurotrophic factor), TMEM158 (transmembrane protein 158 (gene/pseudogene)), and ENSG00000078687.

In a particular aspect, the instant disclosure provides prime editing for the treatment of a subject diagnosed with an expansion repeat disorder (also known as a repeat expansion disorder or a trinucleotide repeat disorder). Expansion repeat disorders occur when microsatellite repeats expand beyond a threshold length. Currently, at least 30 genetic diseases are believed to be caused by repeat expansions. Scientific understanding of this diverse group of disorders came to lights in the early 1990's with the discovery that trinucleotide repeats underlie several major inherited conditions, including Fragile X, Spinal and Bulbar Muscular Atrophy, Myotonic Dystrophy, and Huntington's disease (Nelson et al, “The unstable repeats—three evolving faces of neurological disease,” Neuron, Mar. 6, 2013, Vol. 77; 825-843, which is incorporated herein by reference), as well as Haw River Syndrome, Jacobsen Syndrome, Dentatorubral-pallidoluysian atrophy (DRPLA), Machado-Joseph disease, Synpolydactyly (SPD II), Hand-foot genital syndrome (HFGS), Cleidocranial dysplasia (CCD), Holoprosencephaly disorder (HPE), Congenital central hypventilation syndrome (CCHS), ARX-nonsyndromic X-linked mental retardation (XLMR), and Oculopharyngeal muscular dystrophy (OPMD) (see. Microsatellite repeat instability was found to be a hallmark of these conditions, as was anticipation—the phenomenon in which repeat expansion can occur with each successive generation, which leads to a more severe phenotype and earlier age of onset in the offspring. Repeat expansions are believed to cause diseases via several different mechanisms. Namely, expansions may interfere with cellular functioning at the level of the gene, the mRNA transcript, and/or the encoded protein. In some conditions, mutations act via a loss-of-function mechanism by silencing repeat-containing genes. In others, disease results from gain-of-function mechanisms, whereby either the mRNA transcript or protein takes on new, aberrant functions.

In one embodiment, a method of treating a trinucleotide repeat disorder is depicted in FIG. 23. In general, the approach involves using prime editing in combination with an pegRNA that comprises a region that encodes a desired and healthy replacement trinucleotide repeat sequence that is intended to replace the endogenous diseased trinucleotide repeat sequence through the mechanism of the prime editing process. A schematic of an exemplary gRNA design for contracting trinucleotide repeat sequences and trinucleotide repeat contraction with prime editing is shown in FIG. 23.

### Prion Disease

Prime editing can also be used to prevent or halt the progression of prion disease through the installation of one or more protective mutations into prion proteins (PRNP) which become misfolded during the course of disease. Prion diseases or transmissible spongiform encephalopathies (TSEs) are a family of rare progressive neurodegenerative disorders that affect both humans and animals. They are distinguished by long incubation periods, characteristic spongiform changes associated with neuronal loss, and a failure to induce inflammatory response.

In humans, prion disease includes Creutzfeldt-Jakob Disease (CJD), Variant Creutzfeldt-Jakob Disease (vCJD), Gerstmann-Straussler-Scheinker Syndrome, Fatal Familial Insomnia, and Kuru. In animals, prion disease includes Bovine Spongiform Encephalopathy (BSE or “mad cow disease”), Chronic Wasting Disease (CWD), Scrapie, Transmissible Mink Encephalopathy, Feline Spongiform Encephalopathy, and Ungulate Spongiform Encephalopathy. Prime editing may be used to install protective point mutations into a prion protein in order to prevent or halt the progression of any one of these prion diseases.

Classic CJD is a human prion disease. It is a neurodegenerative disorder with characteristic clinical and diagnostic features. This disease is rapidly progressive and always fatal. Infection with this disease leads to death usually within 1 year of onset of illness. CJD is a rapidly progressive, invariably fatal neurodegenerative disorder believed to be caused by an abnormal isoform of a cellular glycoprotein known as the prion protein. CJD occurs worldwide and the estimated annual incidence in many countries, including the United States, has been reported to be about one case per million population. The vast majority of CJD patients usually die within 1 year of illness onset. CJD is classified as a transmissible spongiform encephalopathy (TSE) along with other prion diseases that occur in humans and animals. In about 85% of patients, CJD occurs as a sporadic disease with no recognizable pattern of transmission. A smaller proportion of patients (5 to 15%) develop CJD because of inherited mutations of the prion protein gene. These inherited forms include Gerstmann-Straussler-Scheinker syndrome and fatal familial insomnia. No treatment is currently known for CJD.

Variant Creutzfeldt-Jakob disease (vCJD) is a prion disease that was first described in 1996 in the United Kingdom. There is now strong scientific evidence that the agent responsible for the outbreak of prion disease in cows, bovine spongiform encephalopathy (BSE or ‘mad cow’ disease), is the same agent responsible for the outbreak of vCJD in humans. Variant CJD (vCJD) is not the same disease as classic CJD (often simply called CJD). It has different clinical and pathologic characteristics from classic CJD. Each disease also has a particular genetic profile of the prion protein gene. Both disorders are invariably fatal brain diseases with unusually long incubation periods measured in years, and are caused by an unconventional transmissible agent called a prion. No treatment is currently known for vCJD.

BSE (bovine spongiform encephalopathy or “mad cow disease”) is a progressive neurological disorder of cattle that results from infection by an unusual transmissible agent called a prion. The nature of the transmissible agent is not well understood. Currently, the most accepted theory is that the agent is a modified form of a normal protein known as prion protein. For reasons that are not yet understood, the normal prion protein changes into a pathogenic (harmful) form that then damages the central nervous system of cattle. There is increasing evidence that there are different strains of BSE: the typical or classic BSE strain responsible for the outbreak in the United Kingdom and two atypical strains (H and L strains). No treatment is currently known for BSE.

Chronic wasting disease (CWD) is a prion disease that affects deer, elk, reindeer, sika deer and moose. It has been found in some areas of North America, including Canada and the United States, Norway and South Korea. It may take over a year before an infected animal develops symptoms, which can include drastic weight loss (wasting), stumbling, listlessness and other neurologic symptoms. CWD can affect animals of all ages and some infected animals may die without ever developing the disease. CWD is fatal to animals and there are no treatments or vaccines.

The causative agents of TSEs are believed to be prions. The term “prions” refers to abnormal, pathogenic agents that are transmissible and are able to induce abnormal folding of specific normal cellular proteins called prion proteins that are found most abundantly in the brain. The functions of these normal prion proteins are still not completely understood. The abnormal folding of the prion proteins leads to brain damage and the characteristic signs and symptoms of the disease. Prion diseases are usually rapidly progressive and always fatal.

As used herein, the term “prion” shall mean an infectious particle known to cause diseases (spongiform encephalopathies) in humans and animals. The term “prion” is a contraction of the words “protein” and “infection” and the particles are comprised largely if not exclusively of PRNP^{Sc} molecules encoded by a PRNP gene which expresses PRNP^{C} which changes conformation to become PRNP^{Sc}. Prions are distinct from bacteria, viruses and viroids. Known prions include those which infect animals to cause scrapie, a transmissible, degenerative disease of the nervous system of sheep and goats as well as bovine spongiform encephalopathies (BSE) or mad cow disease and feline spongiform encephalopathies of cats. Four prion diseases, as discussed above, known to affect humans are (1) kuru, (2) Creutzfeldt-Jakob Disease (CJD), (3) Gerstmann-Strassler-Scheinker Disease (GSS), and (4) fatal familial insomnia (FFI). As used herein prion includes all forms of prions causing all or any of these diseases or others in any animals used—and in particular in humans and in domesticated farm animals.

In general, and without wishing to be bound by theory, prior diseases are caused by misfolding of prion proteins. Such diseases—often called deposition diseases—the misfolding of the prion proteins can be accounted for as follows. If A is the normally synthesized gene product that carries out an intended physiologic role in a monomeric or oligomeric state, A* is a conformationally activated form of A that is competent to undergo a dramatic conformational change, B is the conformationally altered state that prefers multimeric assemblies (i.e., the misfolded form which forms depositions) and B. is the multimeric material that is pathogenic and relatively difficult to recycle. For the prion diseases, PRNP^{C} and PRNP^{Sc} correspond to states A and Bₙ where A is largely helical and monomeric and Bₙ is β-rich and multimeric.

It is known that certain mutations in prion proteins can be associated with increased risk of prior disease. Conversely, certain mutations in prion proteins can be protective in nature. See Bagynszky et al., “Characterization of mutations in PRNP (prion) gene and their possible roles in neurodegenerative diseases,” Neuropsychiatr Dis Treat., 2018; 14: 2067-2085, the contents of which are incorporated herein by reference.

PRNP (NCBI RefSeq No. NP_000302.1 (SEQ ID NO: 396))—the human prion protein—is encoded by a 16 kb long gene, located on chromosome 20 (4686151-4701588). It contains two exons, and the exon 2 carries the open reading frame which encodes the 253 amino acid (AA) long PrP protein. Exon 1 is a noncoding exon, which may serve as transcriptional initiation site. The post-translational modifications result in the removal of the first 22 AA N-terminal fragment (NTF) and the last 23 AA C-terminal fragment (CTF). The NTF is cleaved after PrP transport to the endoplasmic reticulum (ER), while the CTF (glycosylphosphatidylinositol [GPI] signal peptide [GPI-SP]) is cleaved by the GPI anchor. GPI anchor could be involved in PrP protein transport. It may also play a role of attachment of prion protein into the outer surface of cell membrane. Normal PrP is composed of a long N-terminal loop (which contains the octapeptide repeat region), two short 3 sheets, three a helices, and a C-terminal region (which contains the GPI anchor). Cleavage of PrP results in a 208 AA long glyocoprotein, anchored in the cell membrane.

The 253 amino acid sequence of PRNP (NP 000302.1) is as follows:

(SEQ ID NO: 396)
MANLGCWMLVLFVATWSDLGLCKKRPKPGGWNTGGSRYPG
QGSPGGNRYPPQGGGGWGQPHGGGWGQPHGGGWGQPHGGG
WGQPHGGGWGQGGGTHSQWNKPSKPKTNMKHMAGAAAAGA
VVGGLGGYMLGSAMSRPIIHFGSDYEDRYYRENMHRYPNQ
VYYRPMDEYSNQNNFVHDCVNITIKQHTVTTTTKGENFTE
TDVKMMERVVEQMCITQYERESQAYYQRGSSMVLFSSPPV.

The 253 amino acid sequence of PRNP (NP_000302.1) is encoded by the following nucleotide sequence (NCBI Ref. Seq No. NM_000311.5, “Homo sapiens prion protein (PRNP), transcript variant 1, mRNA), is as follows:

(SEQ ID NO: 397)
GCGAACCTTGGCTGCTGGATGCTGGTTCTCTTTGTGGCCA
CATGGAGTGACCTGGGCCTCTGCAAGAAGCGCCCGAAGCC
TGGAGGATGGAACACTGGGGGCAGCCGATACCCGGGGCAG
GGCAGCCCTGGAGGCAACCGCTACCCACCTCAGGGCGGTG
GTGGCTGGGGGCAGCCTCATGGTGGTGGCTGGGGGCAGCC
TCATGGTGGTGGCTGGGGGCAGCCCCATGGTGGTGGCTGG
GGACAGCCTCATGGTGGTGGCTGGGGTCAAGGAGGTGGCA
CCCACAGTCAGTGGAACAAGCCGAGTAAGCCAAAAACCAA
CATGAAGCACATGGCTGGTGCTGCAGCAGCTGGGGCAGTG
GTGGGGGGCCTTGGCGGCTACATGCTGGGAAGTGCCATGA
GCAGGCCCATCATACATTTCGGCAGTGACTATGAGGACCG
TTACTATCGTGAAAACATGCACCGTTACCCCAACCAAGTG
TACTACAGGCCCATGGATGAGTACAGCAACCAGAACAACT
TTGTGCACGACTGCGTCAATATCACAATCAAGCAGCACAC
GGTCACCACAACCACCAAGGGGGAGAACTTCACCGAGACC
GACGTTAAGATGATGGAGCGCGTGGTTGAGCAGATGTGTA
TCACCCAGTACGAGAGGGAATCTCAGGCCTATTACCAGAG
AGGATCGAGCATGGTCCTCTTCTCCTCTCCACCTGTGATC
CTCCTGATCTCTTTCCTCATCTTCCTGATAGTGGGATGAG
GAAGGTCTTCCTGTTTTCACCATCTTTCTAATCTTTTTCC
AGCTTGAGGGAGGCGGTATCCACCTGCAGCCCTTTTAGTG
GTGGTGTCTCACTCTTTCTTCTCTCTTTGTCCCGGATAGG
CTAATCAATACCCTTGGCACTGATGGGCACTGGAAAACAT
AGAGTAGACCTGAGATGCTGGTCAAGCCCCCTTTGATTGA
GTTCATCATGAGCCGTTGCTAATGCCAGGCCAGTAAAAGT
ATAACAGCAAATAACCATTGGTTAATCTGGACTTATTTTT
GGACTTAGTGCAACAGGTTGAGGCTAAAACAAATCTCAGA
ACAGTCTGAAATACCTTTGCCTGGATACCTCTGGCTCCTT
CAGCAGCTAGAGCTCAGTATACTAATGCCCTATCTTAGTA
GAGATTTCATAGCTATTTAGAGATATTTTCCATTTTAAGA
AAACCCGACAACATTTCTGCCAGGTTTGTTAGGAGGCCAC
ATGATACTTATTCAAAAAAATCCTAGAGATTCTTAGCTCT
TGGGATGCAGGCTCAGCCCGCTGGAGCATGAGCTCTGTGT
GTACCGAGAACTGGGGTGATGTTTTACTTTTCACAGTATG
GGCTACACAGCAGCTGTTCAACAAGAGTAAATATTGTCAC
AACACTGAACCTCTGGCTAGAGGACATATTCACAGTGAAC
ATAACTGTAACATATATGAAAGGCTTCTGGGACTTGAAAT
CAAATGTTTGGGAATGGTGCCCTTGGAGGCAACCTCCCAT
TTTAGATGTTTAAAGGACCCTATATGTGGCATTCCTTTCT
TTAAACTATAGGTAATTAAGGCAGCTGAAAAGTAAATTGC
CTTCTAGACACTGAAGGCAAATCTCCTTTGTCCATTTACC
TGGAAACCAGAATGATTTTGACATACAGGAGAGCTGCAGT
TGTGAAAGCACCATCATCATAGAGGATGATGTAATTAAAA
AATGGTCAGTGTGCAAAGAAAAGAACTGCTTGCATTTCTT
TATTTCTGTCTCATAATTGTCAAAAACCAGAATTAGGTCA
AGTTCATAGTTTCTGTAATTGGCTTTTGAATCAAAGAATA
GGGAGACAATCTAAAAAATATCTTAGGTTGGAGATGACAG
AAATATGATTGATTTGAAGTGGAAAAAGAAATTCTGTTAA
TGTTAATTAAAGTAAAATTATTCCCTGAATTGTTTGATAT
TGTCACCTAGCAGATATGTATTACTTTTCTGCAATGTTAT
TATTGGCTTGCACTTTGTGAGTATTCTATGTAAAAATATA
TATGTATATAAAATATATATTGCATAGGACAGACTTAGGA
GTTTTGTTTAGAGCAGTTAACATCTGAAGTGTCTAATGCA
TTAACTTTTGTAAGGTACTGAATACTTAATATGTGGGAAA
CCCTTTTGCGTGGTCCTTAGGCTTACAATGTGCACTGAAT
CGTTTCATGTAAGAATCCAAAGTGGACACCATTAACAGGT
CTTTGAAATATGCATGTACTTTATATTTTCTATATTTGTA
ACTTTGCATGTTCTTGTTTTGTTATATAAAAAAATTGTAA
ATGTTTAATATCTGACTGAAATTAAACGAGCGAAGATGAG
CACCA

Mutation sites relative to PRNP (NP_000302.1) which are linked to CJD and FFI are reported are as follows. These mutations can be removed or installed using the prime editors disclosed herein.

| | AMINO ACID SEQUENCE OF MUTANT PRNP |
|---|---|
| | LINKED TO CJD PRION DISEASE (SEE |
| | TABLE 1 OF BAGYNSZKY ET AL., 2018) |
| | (RELATIVE TO SEQ ID NO: 396 OF |
| MUTATION | PRNP NP_000302.1) |
| D178N | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHNCV |
| | NITIKQHTVTTTTKGENFTETDVKMMERVV |
| | EQMCITQYERESQAYYQRGSSMVLFSSPPV |
| | (SEQ ID NO: 398) |
| T188K | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHKVTTTTKGENFTETDVKMMERVV |
| | EQMCITQYERESQAYYQRGSSMVLFSSPPV |
| | (SEQ ID NO: 399) |
| E196K | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGKNFTETDVKMMERVV |
| | EQMCITQYERESQAYYQRGSSMVLFSSPPV |
| | (SEQ ID NO: 400) |
| E196A | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGANFTETDVKMMERVV |
| | EQMCITQYERESQAYYQRGSSMVLFSSPPV |
| | (SEQ ID NO: 401) |
| E200K | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTKTDVKMMERVV |
| | EQMCITQYERESQAYYQRGSSMVLFSSPPV |
| | (SEQ ID NO: 402) |
| E200G | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTGTDVKMMERVV |
| | EQMCITQYERESQAYYQRGSSMVLFSSPPV |
| | (SEQ ID NO: 403) |
| V203I | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDIKMMERVV |
| | EQMCITQYERESQAYYQRGSSMVLFSSPPV |
| | (SEQ ID NO: 404) |
| R208H | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMEHVV |
| | EQMCITQYERESQAYYQRGSSMVLFSSPPV |
| | (SEQ ID NO: 405) |
| V210I | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVI |
| | EQMCITQYERESQAYYQRGSSMVLFSSPPV |
| | (SEQ ID NO: 406) |
| E211Q | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | QQMCITQYERESQAYYQRGSSMVLFSSPPV |
| | (SEQ ID NO: 407) |
| M232R | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSRVLFSSPPV |
| | (SEQ ID NO: 408) |

Mutation sites relative to PRNP (NP_000302.1) (SEQ ID NO: 396) which are linked to GSS are reported, as follows:

| | AMINO ACID SEQUENCE OF MUTANT |
|---|---|
| | PRNP LINKED TO GSS PRION |
| | DISEASE (SEE TABLE 2 OF |
| | BAGYNSZKY ET AL., 2018) |
| | (RELATIVE TO SEQ ID NO: 396 |
| MUTATION | OF PRNP NP_000302.1) |
| P102L | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKLSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 409) |
| P105L | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKLKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 410) |
| A117V | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAVAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 411) |
| G131V | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLVSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 412) |
| V176G | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFGHDCV |
| | NITIKQHTVTTTTKGENFTETDVKMMERVV |
| | EQMCITQYERESQAYYQRGSSMVLFSSPPV |
| | (SEQ ID NO: 413) |
| H187R | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQRTVTTTTKGENFTETDVKMMERVV |
| | EQMCITQYERESQAYYQRGSSMVLFSSPPV |
| | (SEQ ID NO: 414) |
| | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 396) |
| F198S | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENSTETDVKMMERVV |
| | EQMCITQYERESQAYYQRGSSMVLFSSPPV |
| | (SEQ ID NO: 415) |
| D202N | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETNVKMMERVV |
| | EQMCITQYERESQAYYQRGSSMVLFSSPPV |
| | (SEQ ID NO: 416) |
| Q212P | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EPMCITQYERESQAYYQRGSSMVLFSSPPV |
| | (SEQ ID NO: 417) |
| Q217R | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITRYERESQAYYQRGSSMVLFSSPPV |
| | (SEQ ID NO: 418) |
| M232T | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSTVLFSSPPV |
| | (SEQ ID NO: 419) |

Mutation sites relative to PRNP (NP_000302.1) (SEQ ID NO: 396) which are linked to a possible protective nature against prion disease, as follows:

| | AMINO ACID SEQUENCE OF MUTANT PRNP |
|---|---|
| | LINKED TO A PROTECTIVENATURE AGAINST |
| | PRION DISEASE (SEE TABLE 4 OF |
| | BAGYNSZKY ET AL., 2018) (RELATIVE |
| | TO SEQ ID NO: 396 OF |
| MUTATION | PRNP NP_000302.1) |
| G127S | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGSYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 420) |
| G127V | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGVYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 421) |
| M129V | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYVLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSPPV |
| | (SEQ ID NO: 422) |
| D167G | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMGEYSNQNNFVHDCV |
| | NITIKQHTVTTTTKGENFTETDVKMMERVV |
| | EQMCITQYERESQAYYQRGSSMVLFSSPPV |
| | (SEQ ID NO: 423) |
| D167N | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMNEYSNQNNFVHDCV |
| | NITIKQHTVTTTTKGENFTETDVKMMERVV |
| | EQMCITQYERESQAYYQRGSSMVLFSSPPV |
| | (SEQ ID NO: 424) |
| N171S | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSSQNNFVHDCV |
| | NITIKQHTVTTTTKGENFTETDVKMMERVV |
| | EQMCITQYERESQAYYQRGSSMVLFSSPPV |
| | (SEQ ID NO: 425) |
| E219K | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYKRESQAYYQRGSSMVLFSSPPV |
| | (SEQ ID NO: 426) |
| P238S | MANLGCWMLVLFVATWSDLGLCKKRPKPGG |
| | WNTGGSRYPGQGSPGGNRYPPQGGGGWGQP |
| | HGGGWGQPHGGGWGQPHGGGWGQPHGGGWG |
| | QGGGTHSQWNKPSKPKTNMKHMAGAAAAGA |
| | VVGGLGGYMLGSAMSRPIIHFGSDYEDRYY |
| | RENMHRYPNQVYYRPMDEYSNQNNFVHDCV |
| | NITIKQHTVTTTTKGENFTETDVKMMERVV |
| | EQMCITQYERESQAYYQRGSSMVLFSSSPV |
| | (SEQ ID NO: 427) |

Thus, in various embodiments, prime editing may be used to remove a mutation in PRNP that is linked to prion disease or install a mutation in PRNP that is considered to be protective against prion disease. For example, prime editing may be use to remove or restore a D178N, V1801, T188K, E196K, E196A, E200K, E200G, V203I, R208H, V210I, E211Q, I215V, or M232R mutation in the PRNP protein (relative to PRNP of NP_000302.1) (SEQ ID NO: 396). In other embodiments, prime editing may be use to remove or restore a P102L, P105L, A117V, G131V, V176G, H187R, F198S, D202N, Q212P, Q217R, or M232T mutation in the PRNP protein (relative to PRNP of NP_000302.1) (SEQ ID NO: 396). By removing or correcting for the presence of such mutations in PRNP using prime editing, the risk of prion disease may be reduced or eliminated.

In other embodiments, prime editing may be used to install a protective mutation in PRNP that is linked to a protective effect against one or more prion diseases. For example, prime editing may be used to install a G127S, G127V, M129V, D167G, D167N, N171S, E219K, or P238S protective mutation in PRNP (relative to PRNP of NP_000302.1) (SEQ ID NO: 396). In still other embodiments, the protective mutation may be any alternate amino acid installed at G127, G127, M129, D167, D167, N171, E219, or P238 in PRNP (relative to PRNP of NP_000302.1) (SEQ ID NO: 396).

In particular embodiments, prime editing may be used to install a G127V protective mutation in PRNP, as illustrated in FIG. 27 and discussed in Example 5.

In another embodiment, prime editing may be used to install an E219K protective mutation in PRNP.

The PRNP protein and the protective mutation site are conserved in mammals, so in addition to treating human disease it could also be used to generate cows and sheep that are immune to prion disease, or even help cure wild populations of animals that are suffering from prion disease. Prime editing can be used to achieve ˜25% installation of a naturally occurring protective allele in human cells, and mouse experiments indicate that this level of installation is sufficient to cause immunity from prion diseases. This method is the first and potentially only current way to install this allele with such high efficiency in most cell types. Another possible strategy for treatment is to use prime editing to reduce or eliminate the expression of PRNP by installing an early stop codon in the gene.

Using the principles described herein for pegRNA design, appropriate pegRNAs may be designed for installing desired protective mutations, or for removing prion disease-associated mutations from PRNP. For example, the below list of pegRNAs can be used to install the G127V protective allele and the E219K protective allele in human PRNP, as well as the G127V protective allele in PRNP of various animals.

### [10] Pharmaceutical Compositions

Other aspects of the present disclosure relate to pharmaceutical compositions comprising any of the various components of the prime editing system described herein (e.g., including, but not limited to, the napDNAbps, reverse transcriptases, fusion proteins (e.g., comprising napDNAbps and reverse transcriptases), pegRNAs, and complexes comprising fusion proteins and pegRNAs, as well as accessory elements, such as second strand nicking components and 5′ endogenous DNA flap removal endonucleases for helping to drive the prime editing process towards the edited product formation).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits, Cells, Vectors, and Delivery

### Kits

The compositions of the present disclosure may be assembled into kits. In some embodiments, the kit comprises nucleic acid vectors for the expression of the prime editors described herein. In other embodiments, the kit further comprises appropriate guide nucleotide sequences (e.g., pegRNAs and second-site gRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the prime editing system described herein (e.g., including, but not limited to, the napDNAbps, reverse transcriptases, polymerases, fusion proteins (e.g., comprising napDNAbps and reverse transcriptases (or more broadly, polymerases), pegRNAs, and complexes comprising fusion proteins and pegRNAs, as well as accessory elements, such as second strand nicking components (e.g., second strand nicking gRNA) and 5′ endogenous DNA flap removal endonucleases for helping to drive the prime editing process towards the edited product formation). In some embodiments, the nucleotide sequence(s) comprises a heterologous promoter (or more than a single promoter) that drives expression of the prime editing system components.

Other aspects of this disclosure provide kits comprising one or more nucleic acid constructs encoding the various components of the prime editing system described herein, e.g., the comprising a nucleotide sequence encoding the components of the prime editing system capable of modifying a target DNA sequence. In some embodiments, the nucleotide sequence comprises a heterologous promoter that drives expression of the prime editing system components.

Some aspects of this disclosure provides kits comprising a nucleic acid construct, comprising (a) a nucleotide sequence encoding a napDNAbp (e.g., a Cas9 domain) fused to a polymerase, such as a reverse transcriptase and (b) a heterologous promoter that drives expression of the sequence of (a).

### Cells

Cells that may contain any of the compositions described herein include prokaryotic cells and eukaryotic cells. The methods described herein are used to deliver a Cas9 protein or a prime editor into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-1OT1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Vectors

Some aspects of the present disclosure relate to using recombinant virus vectors (e.g., adeno-associated virus vectors, adenovirus vectors, or herpes simplex virus vectors) for the delivery of the prime editors or components thereof described herein, e.g., the split Cas9 protein or a split nucleobase prime editors, into a cell. In the case of a split-PE approach, the N-terminal portion of a Prime editor and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or prime editors exceeds the packaging limit of various virus vectors, e.g., rAAV (˜4.9 kb).

Thus, in one embodiment, the disclosure contemplates vectors capable of delivering split prime editor, or split components thereof. In some embodiments, a composition for delivering the split Cas9 protein or split prime editor into a cell (e.g., a mammalian cell, a human cell) is provided. In some embodiments, the composition of the present disclosure comprises: (i) a first recombinant adeno-associated virus (rAAV) particle comprising a first nucleotide sequence encoding a N-terminal portion of a Cas9 protein or prime editor fused at its C-terminus to an intein-N; and (ii) a second recombinant adeno-associated virus (rAAV) particle comprising a second nucleotide sequence encoding an intein-C fused to the N-terminus of a C-terminal portion of the Cas9 protein or prime editor. The rAAV particles of the present disclosure comprise a rAAV vector (i.e., a recombinant genome of the rAAV) encapsulated in the viral capsid proteins.

In some embodiments, the rAAV vector comprises: (1) a heterologous nucleic acid region comprising the first or second nucleotide sequence encoding the N-terminal portion or C-terminal portion of a split Cas9 protein or a split prime editor in any form as described herein, (2) one or more nucleotide sequences comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of a cell. In some embodiments, viral sequences that facilitate integration comprise Inverted Terminal Repeat (ITR) sequences. In some embodiments, the first or second nucleotide sequence encoding the N-terminal portion or C-terminal portion of a split Cas9 protein or a split prime editor is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6.

Thus, in some embodiments, the rAAV particles disclosed herein comprise at least one rAAV2 particle, rAAV6 particle, rAAV8 particle, rPHP.B particle, rPHP.eB particle, or rAAV9 particle, or a variant thereof. In particular embodiments, the disclosed rAAV particles are rPHP.B particles, rPHP.eB particles, rAAV9 particles.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators of the bovine growth hormone gene (bGH), human growth hormone gene (hGH), SV40, CW3, #, or combinations thereof. The efficiencies of several transcriptional terminators have been tested to determine their respective effects in the expression level of the split Cas9 protein or the split prime editor. In some embodiments, the transcriptional terminator used in the present disclosure is a bGH transcriptional terminator. In some embodiments, the rAAV vector further comprises a Woodchuck Hepatitis Virus Posttranscriptional Regulatory Element (WPRE). In certain embodiments, the WPRE is a truncated WPRE sequence, such as “W3.” In some embodiments, the WPRE is inserted 5′ of the transcriptional terminator. Such sequences, when transcribed, create a tertiary structure which enhances expression, in particular, from viral vectors.

In some embodiments, the vectors used herein may encode the Prime editors, or any of the components thereof (e.g., napDNAbp, linkers, or polymerases). In addition, the vectors used herein may encode the pegRNAs, and/or the accessory gRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the prime editor vectors (e.g., including any vectors encoding the prime editor and/or the pegRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor and/or the pegRNAs, and/or the accessory second strand nicking gRNAs) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the nucleotide sequence encoding the pegRNA (or any guide RNAs used in connection with prime editing) may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the Prime editor. In some embodiments, expression of the guide RNA and of the Prime editor may be driven by their corresponding promoters. In some embodiments, expression of the guide RNA may be driven by the same promoter that drives expression of the Prime editor. In some embodiments, the guide RNA and the Prime editor transcript may be contained within a single transcript. For example, the guide RNA may be within an untranslated region (UTR) of the Cas9 protein transcript. In some embodiments, the guide RNA may be within the 5′ UTR of the Prime editor transcript. In other embodiments, the guide RNA may be within the 3′ UTR of the Prime editor transcript. In some embodiments, the intracellular half-life of the Prime editor transcript may be reduced by containing the guide RNA within its 3′ UTR and thereby shortening the length of its 3′ UTR. In additional embodiments, the guide RNA may be within an intron of the Prime editor transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The prime editor vector system may comprise one vector, or two vectors, or three vectors, or four vectors, or five vector, or more. In some embodiments, the vector system may comprise one single vector, which encodes both the Prime editor and pegRNA. In other embodiments, the vector system may comprise two vectors, wherein one vector encodes the Prime editor and the other encodes the pegRNA. In additional embodiments, the vector system may comprise three vectors, wherein the third vector encodes the second strand nicking gRNA used in the herein methods.

In some embodiments, the composition comprising the rAAV particle (in any form contemplated herein) further comprises a pharmaceutically acceptable carrier. In some embodiments, the composition is formulated in appropriate pharmaceutical vehicles for administration to human or animal subjects.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Delivery Methods

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

Exemplary delivery strategies are described herein elsewhere, which include vector-based strategies, PE ribonucleoprotein complex delivery, and delivery of PE by mRNA methods.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electroporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of fusion proteins markedly increases the DNA specificity of base editing. RNP delivery of fusion proteins leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

Additional methods for the delivery of nucleic acids to cells are known to those skilled in the art. See, for example, US 2003/0087817, incorporated herein by reference.

Other aspects of the present disclosure provide methods of delivering the prime editor constructs into a cell to form a complete and functional prime editor within a cell. For example, in some embodiments, a cell is contacted with a composition described herein (e.g., compositions comprising nucleotide sequences encoding the split Cas9 or the split prime editor or AAV particles containing nucleic acid vectors comprising such nucleotide sequences). In some embodiments, the contacting results in the delivery of such nucleotide sequences into a cell, wherein the N-terminal portion of the Cas9 protein or the prime editor and the C-terminal portion of the Cas9 protein or the prime editor are expressed in the cell and are joined to form a complete Cas9 protein or a complete prime editor.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a split protein), or transfected (e.g., with a plasmid encoding a split protein) with a nucleic acid molecule that encodes a split protein, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences, for example in delivery of a split Cas9 (e.g., nCas9) protein. In some embodiments, a plasmid expressing a split protein may be introduced into cells through electroporation, transient (e.g., lipofection) and stable genome integration (e.g., piggybac) and viral transduction or other methods known to those of skill in the art.

In certain embodiments, the compositions provided herein comprise a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

The guide RNA sequence may be 15-100 nucleotides in length and comprise a sequence of at least 10, at least 15, or at least 20 contiguous nucleotides that is complementary to a target nucleotide sequence. The guide RNA may comprise a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target nucleotide sequence. The guide RNA may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the target nucleotide sequence is a DNA sequence in a genome, e.g. a eukaryotic genome. In certain embodiments, the target nucleotide sequence is in a mammalian (e.g. a human) genome.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

Treatment of a disease or disorder includes delaying the development or progression of the disease, or reducing disease severity. Treating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EMBODIMENTS

The disclosure further relates to the following non-limiting numbered paragraphs.
1. A prime editing guide RNA (PEgRNA) comprising:
   a spacer sequence that comprises a region of complementarity to a target strand of a double-stranded target DNA sequence;
   a nucleic acid extension arm comprising a DNA synthesis template core that associates with a nucleic acid programmable DNA binding protein (napDNAbp), wherein the primer binding site comprises a region of complementarity to a non-target strand of the double-stranded target DNA sequence;
   wherein the DNA synthesis template comprises a region of complementarity to the non-target strand of the double-stranded target DNA sequence and comprises one or more nucleotide edits compared to the double-stranded target DNA sequence; and wherein the extension arm further comprises a nucleic acid moiety selected from the group consisting of a toe-loop, hairpin, stem-loop, pseudoknot, aptamer. G-quadraplex, tRNA, riboswitch, or ribozyme.
2. The PEgRNA of paragraph 1, wherein the nucleic acid moiety is at the 3′ end of the extension arm.
3. The PEgRNA of paragraph 1, wherein the nucleic acid moiety is at the 5′ end of the extension arm.
4. The PEgRNA of paragraph 1, wherein the nucleic acid moiety comprises a frameshifting pseudoknot from a Moloney murine leukemia virus (M-MLV) genome (a Mpknot), optionally wherein the Mpknot is a Mpknot1 moiety having a nucleotide sequence selected from the group consisting of: SEQ ID NO: 3930 (Mpknot1), SEQ ID NO: 3931 (Mpknot1 3′ trimmed). SEQ ID NO: 3932 (Mpknot1 with 5′ extra), SEQ ID NO: 3933 (Mpknot1 U38A), SEQ ID NO: 3934 (Mpknot1 U38A A29C). SEQ ID NO: 3935 (MMLC A29C), SEQ ID NO: 3936 (Mpknot1 with 5′ extra and U38A). SEQ ID NO: 3937 (Mpknot1 with 5′ extra and U38A A29C), and SEQ ID NO: 3938 (Mpknot1 with 5′ extra and A29C), or a nucleotide sequence having at least 80% sequence identity therewith.
5. The PEgRNA of paragraph 1, wherein the nucleic acid moiety comprises a G-quadruplex, optionally wherein the G-quadruplex has a nucleotide sequence selected from the group consisting of: SEQ ID NO: 3939 (tns1), SEQ ID NO: 3940 (stk40). SEQ ID NO: 3941 (apc2). SEQ ID NO: 3942 (ceacam4). SEQ ID NO: 3943 (pitpnm3), SEQ ID NO: 3944 (rlf), SEQ ID NO: 3945 (erc1). SEQ ID NO: 3946 (ube3c), SEQ ID NO: 3947 (taf15), SEQ ID NO: 3948 (stard3), and SEQ ID NO: 3949 (g2), or a nucleotide sequence having at least 80% sequence identity therewith.
6. The PEgRNA of paragraph 1, wherein the nucleic acid moiety comprises a prequeosine1 riboswitch aptamer.
7. The PEgRNA of paragraph 6, wherein the nucleic acid moiety comprises an evolved prequeosine1-1 riboswitch aptamer (evopreQ1), optionally wherein the evopreQ1 has a nucleotide sequence selected from the group consisting of: SEQ ID NO: 3950 (evopreq1), SEQ ID NO: 3951 (evopreq1motif1), SEQ ID NO: 3952 (evopreq1motif2). SEQ ID NO: 3953 (evopreq1motif3), SEQ ID NO: 3954 (shorter preq1-1), SEQ ID NO: 3955 (preq1-1 G5C (mut1)), and SEQ ID NO: 3956 (preq1-1 G15C (mut2)), or a nucleotide sequence having at least 80% sequence identity therewith.
8. The PEgRNA of paragraph 1, wherein the nucleic acid moiety comprises a tRNA moiety having a nucleotide sequence of SEQ ID NO: 3957, or a nucleotide sequence having at least 80% sequence identity therewith.
9. The PEgRNA of paragraph 1, wherein the nucleic acid moiety has a nucleotide sequence of SEQ ID NO: 3958 (xrn1), or a nucleotide sequence having at least 80% sequence identity therewith.
10. The PEgRNA of paragraph 1, wherein the nucleic acid moiety comprises a P4-P6 domain of a group I intron, optionally wherein the P4-P6 domain has a nucleotide sequence of SEQ ID NO: 3959, or a nucleotide sequence having at least 80% sequence identity therewith.
11. The PEgRNA of any of paragraphs 1-10, wherein the PEgRNA further comprises a linker.
12. The PEgRNA of paragraph 11, wherein the linker is between the nucleic acid moiety and another component of the PEgRNA.
13. The PEgRNA of paragraph 11, wherein the linker is between the nucleic acid moiety and the primer binding site or between the gRNA core and the nucleic acid moiety. PEgRNA
14. The PEgRNA of paragraph 11, wherein the linker comprises a nucleotide sequence selected from the group consisting of SEQ ID NO: 3960, SEQ ID NO: 3961. SEQ ID NO: 3962. SEQ ID NO: 3963, SEQ ID NO: 3964. SEQ ID NO: 3965, SEQ ID NO: 3966. SEQ ID NO: 3967. SEQ ID NO: 3968, SEQ ID NO: 3969. SEQ ID NO: 3970, and SEQ ID NO: 3971.
15. The PEgRNA of paragraph 11 wherein the linker is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides in length, wherein the linker is no longer than 50 nucleotides.
16. The PEgRNA of paragraph 11, wherein the linker is 1 to 5 nucleotides, 5 to 10 nucleotides, 10 to 20 nucleotides, 15 to 25 nucleotides, 20 to 30 nucleotides, 25 to 35 nucleotides, 30 to 40 nucleotides, 35 to 45 nucleotides, or 40 to 50 nucleotides in length; or wherein the linker is 1 to 50, 3 to 50, 5 to 50, or 8 to 50 nucleotides in length.
17. The PEgRNA of paragraph 11, wherein the linker is 8 nucleotides in length.
18. The PEgRNA of any one of paragraphs 4-17, wherein the extension arm is positioned at the 3′ or 5′ end of the guide RNA, and wherein the nucleic acid extension arm comprises DNA or RNA.
19. The PEgRNA of paragraph 18, wherein the primer binding site comprises a region of complementarity to a region upstream of a nick site in the non-target strand of the target DNA sequence, wherein the nick site is characteristic of the napDNAbp.
20. The PEgRNA of paragraph 19, wherein the DNA synthesis template comprises a region of complementarity to a region downstream of the nick site in the non-target strand of the target DNA sequence.
21. The PEgRNA of paragraph 18, wherein primer binding site comprises a region of complementarity to a region immediately upstream of a nick site in the non-target strand of the target DNA sequence.
22. The PEgRNA of paragraph 18, wherein the nucleic acid extension arm is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides; or wherein the nucleic acid extension arm is 10 to 20, 20 to 30, 30 to 40.40 to 50, 50 to 60, 60 to 70, 70 to 80, 80 to 90, 90 to 100, 100 to 110, 110 to 120, 20 to 120, 40 to 120, 60 to 120, 80 to 120, 100 to 120, 40 to 100, 60 to 100, 80 to 100, or 60 to 80 nucleotides in length; or wherein the nucleic acid extension arm is 15 to 300, 20 to 250, 20 to 200, 20 to 150, 25 to 150, 15 to 100, 20 to 100 or 25 to 100 nucleotides in length; or wherein the nucleic acid extension arm is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.
23. The PEgRNA of paragraph 18, wherein the DNA synthesis template is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length; or wherein the DNA synthesis template is 1 to 10, 5 to 15, 10 to 20, 20 to 30, 30 to 40, 40 to 50, 50 to 60, 60 to 70, 70 to 80, 80 to 90, 90 to 100, 10 to 30, 10 to 40, 10 to 50, 10 to 60, 20 to 40, 20 to 60, 30 to 100, 40 to 100, 50 to 100, 60 to 100, or 70 to 100 nucleotides in length; wherein the DNA synthesis template is 5 to 300, 5 to 250, 15 to 200, 15 to 150, 5 to 100, 10 to 100, or 15 to 100 nucleotides in length; or wherein the DNA synthesis template is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length.
24. The PEgRNA of paragraph 23, wherein the DNA synthesis template is from 15 to 35 nucleotides in length.
25. The PEgRNA of paragraph 18, wherein the primer binding site is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length, or wherein the primer binding site is 1 to 10 nucleotides, 5 to 10 nucleotides, 10 to 15 nucleotides, 10 to 20 nucleotides, 8 to 20 nucleotides, 15 to 25 nucleotides, 20 to 30 nucleotides, or 25 to 30 nucleotides in length; wherein the primer binding site is 3 to 60, 5 to 60, 8 to 50, or 12 to 50 nucleotides in length, or wherein the primer binding site is 1, 2, 3, 4, 5, 6, 7, 8, 9, 10, 11, 12, 13, 14, 15, 16, 17, 18, 19, 20, 21, 22, 23, 24, or 25 nucleotides in length.
26. The PEgRNA of any one of paragraphs 1-25, wherein the gRNA core comprises a direct repeat, wherein the direct repeat does not contain four or more consecutive A-U base pairs.
27. The PEgRNA of paragraph 26, wherein the direct repeat comprises the nucleotide sequence UUUA.
28. The PEgRNA of any one of paragraphs 1-27, wherein the PEgRNA comprises a chemically or biologically modified nucleotide or a nucleotide analog.
29. The PEgRNA of paragraph 28, wherein the three consecutive nucleotides at the 5′ end of the PEgRNA comprises one or more chemically modified nucleotide, and/or wherein the three consecutive nucleotides at the 3′ end of the PEgRNA comprises one or more chemically modified nucleotide.
30. A prime editor system comprising:
(a) a nucleic acid programmable DNA binding protein (napDNAbp)
(b) a domain comprising an DNA polymerase activity; and
(c) a PEgRNA of any one of paragraphs 1-29.
31. The prime editor system of paragraph 30, wherein the PEgRNA and the napDNAbp and/or the domain comprising DNA polymerase activity form a complex.
32. The prime editor system of paragraph 30 or 31, wherein the domain having DNA polymerase activity and the napDNAbp are fused to form a fusion protein.
33. The prime editor system of any one of paragraphs 30-32, wherein the napDNAbp has a nickase activity.
34. The prime editor system of any one of paragraphs 30-32, wherein the napDNAbp is a Cas9 protein or variant thereof.
35. The prime editor system of paragraph 34, wherein the napDNAbp is a nuclease active Cas9, a nuclease inactive Cas9 (dCas9), or a Cas9 nickase (nCas9).
36. The prime editor system of paragraph 35, wherein the napDNAbp is Cas9 nickase (nCas9).
37. The prime editor system of any one of paragraphs 30-32, wherein the napDNAbp is selected from the group consisting of: Cas9, Cas12e, Cas12d, Cas12a. Cas12b1. Cas13a, Cas12c, and Argonaute and optionally has a nickase activity.
38. The prime editor system of any one of paragraphs 30-37, wherein the domain comprising an RNA-dependent DNA polymerase activity is a reverse transcriptase comprising an amino acid sequence having at least 80%, 85%, 90%, 95%, 98%, or 99% sequence identity with the amino acid sequence of any one of SEQ ID NOs: 89-100, 105-122, 128-129, 132, 139, 143, 149, 154, 159, 235, 454.471, 516, 662, 700.701-716.739-741, and 766.
39. The prime editor system of any one of paragraphs 30-37, wherein the domain comprising an RNA-dependent DNA polymerase activity is a reverse transcriptase comprising any one of the amino acid sequences of SEQ ID NO: 89-100, 105-122, 128-129, 132, 139, 143, 149, 154, 159, 235.454, 471, 516, 662.700, 701-716, 739-741, and 766.
40. The prime editor system of paragraph 38, wherein the reverse transcriptase is a Moloney-Murine Leukemia Virus reverse transcriptase (M-MLVRT).
41. The prime editor system of paragraph 40, wherein the RNA-dependent DNA polymerase domain comprises a variant Moloney-Murine Leukemia Virus reverse transcriptase (M-MLV RT) domain, wherein the variant M-MLV RT domain comprises one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X relative to the amino acid sequence of SEQ ID NO: 89, and wherein X is any amino acid.
42. The prime editor system of paragraph 41, wherein the variant M-MLV RT domain comprises one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N relative to the amino acid sequence of SEQ ID NO: 89.
43. The prime editor system of paragraph 41, wherein the variant M-MLV RT domain comprises an amino acid substitutions D200N, T330P and L603W relative to the amino acid sequence of SEQ ID NO: 89, optionally wherein the M-MLV RT domain comprises amino acid substitutions D200N, T306K, W313F, T330P, and L603W relative to the amino acid sequence of SEQ ID NO: 89.
44. The prime editor system of any one of paragraphs 30-37, wherein the domain comprising an RNA-dependent DNA polymerase activity is a naturally-occurring reverse transcriptase from a retrovirus or a retrotransposon.
45. A nucleic acid molecule encoding the PEgRNA of any one of paragraph 1-29.
46. A nucleic acid molecule encoding the napDNAbp and/or the domain having DNA polymerase activity of any one of paragraphs 30-45.
47. An expression vector comprising the nucleic acid molecule of paragraph 45 and/or the nucleic acid molecule of paragraph 46, optionally wherein the nucleic acid molecule is under the control of a promoter.
48. The expression vector of paragraph 47, wherein the promoter is a polI promoter.
49. The expression vector of paragraph 47, wherein the promoter is a U6 promoter.
50. The expression vector of paragraph 47, wherein the promoter is a U6. U6v4. U6v7, or U6v9 promoter or a fragment thereof.
51. A cell comprising the PEgRNA of any one of paragraphs 1-29.
52. A cell comprising the prime editor system of any one of paragraphs 30-44, the nucleic acid molecule of paragraph 45 or 46, or the expression vector of any one of paragraphs 47-50.
53. A lipid nanoparticle (LNP) comprising the PEgRNA of any one of paragraphs 1-29, the prime editor system of any one of paragraphs 30-44, or the nucleic acid molecule of paragraph 45 or 46.
54. A ribonucleoprotein complex (RNP) comprising the PEgRNA of any one of paragraphs 1-29, the prime editor system of any one of paragraphs 30-44, or the nucleic acid molecule of paragraph 45 or 46.
55. A pharmaceutical composition comprising: (i) PEgRNA of any one of paragraphs 1-29, the prime editor system of any one of paragraphs 30-44, or the nucleic acid molecule of paragraph 45 or 46PEgRNA, the expression vector of any one of paragraphs 47-50, the cell of paragraph 51 or 52, the LNP of paragraph 53, or the RNP of paragraph 54, and (ii) a pharmaceutically acceptable excipient.
56. A kit composition comprising: (i) the PEgRNA of any one of paragraphs 1-29, the prime editor system of any one of paragraphs 30-44, or the nucleic acid molecule of paragraph 45 or 46, the expression vector of any one of paragraphs 47-50, the cell of paragraph 51 or 52, the LNP of paragraph 53, or the RNP of paragraph 54 PEgRNA (ii) a set of instructions for conducting prime editing.
57. A method of prime editing comprising contacting a target DNA sequence with a PEgRNA of any of paragraphs 1-29 and a prime editor comprising a napDNAbp and a domain having a DNA polymerase activity, wherein the contacting installs one or more nucleotide edits in the target DNA sequence.
58. The method of paragraph 57, wherein the editing efficiency is increased as compared to the editing efficiency when the target DNA is contacted with the prime editor and a control PEgRNA that does not contain the nucleic acid moiety PEgRNA.
59. The method of paragraph 58, wherein the editing efficiency is increased by at least 1.5 fold.
60. The method of paragraph 58, wherein the editing efficiency is increased by at least 2 fold.
61. The method of paragraph 58, wherein the editing efficiency is increased by at least 3 fold, 4-fold, 5-fold, 6-fold, 7-fold, 8-fold, 9-fold, or 10-fold [1255], [1270].
62. The method of any one of paragraphs 57-61, wherein the napDNAbp has a nickase activity.
63. The method of carry one of paragraphs 57-62, wherein the napDNAbp is a Cas9 protein or variant thereof.
64. The method of paragraph 63, wherein the napDNAbp is a nuclease active Cas9, a nuclease inactive Cas9 (dCas9), or a Cas9 nickase (nCas9).
65. The method of paragraph 64, wherein the napDNAbp is Cas9 nickase (nCas9).
66. The method of any one of paragraphs 57-62, wherein the napDNAbp is selected from the group consisting of: Cas9, Cas12e, Cas12d, Cas12a, Cas12b1, Cas13a, Cas12c, and Argonaute and optionally has a nickase activity.
67. The method of any one of paragraphs 57-66, wherein the domain comprising an RNA-dependent DNA polymerase activity is a reverse transcriptase comprising any one of the amino acid sequences of SEQ ID NO: 89-100, 105-122, 128-129, 132, 139, 143, 149, 154, 159, 235, 454, 471, 516, 662, 700, 701-716, 739-741, and 766.
68. The method of any one of paragraphs 57-66, wherein the domain comprising an RNA-dependent DNA polymerase activity is a reverse transcriptase comprising an amino acid sequence having at least 80%, 85%, 90%, 95%, 98%, or 99% sequence identity with the amino acid sequence of any one of SEQ ID NOs: 89-100, 105-122, 128-129, 132, 139, 143, 149, 154, 159, 235, 454, 471, 516, 662, 700, 701-716, 739-741, and 766.
69. The method of paragraph 68, wherein the reverse transcriptase is a Moloney-Murine Leukemia Virus reverse transcriptase (M-MLVRT).
70. The method of paragraph 69, wherein the RNA-dependent DNA polymerase domain comprises a variant Moloney-Murine Leukemia Virus reverse transcriptase (M-MLV RT) domain, wherein the variant M-MLV RT domain comprises one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X relative to the amino acid sequence of SEQ ID NO: 89, and wherein X is any amino acid.
71. The method of paragraph 70, wherein the variant M-MLV RT domain comprises one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N relative to the amino acid sequence of SEQ ID NO: 89.
72. The method of paragraph 70, wherein the variant M-MLV RT domain comprises an amino acid substitutions D200N, T330P and L603W relative to the amino acid sequence of SEQ ID NO: 89, optionally wherein the M-MLV RT domain comprises amino acid substitutions D200N, T306K, W313F, T330P, and L603W relative to the amino acid sequence of SEQ ID NO: 89.
73. The method of any one of paragraphs 57-66, wherein the domain comprising an RNA-dependent DNA polymerase activity is a naturally-occurring reverse transcriptase from a retrovirus or a retrotransposon.
74. A method for installing a nucleotide edit in a double stranded target DNA sequence, the method comprising: contacting the double stranded target DNA sequence with a prime editor comprising a nucleic acid programmable DNA binding protein (napDNAbp), a DNA polymerase, and a prime editing guide RNA (PEgRNA), wherein the PEgRNA comprises:
(a) a spacer sequence that comprises a region of complementarity that hybridizes to a target strand of a double-stranded target DNA sequence;
(b) a nucleic acid extension arm comprising a DNA synthesis template and a primer binding site.
(c) a gRNA core that associates with a nucleic acid programmable DNA binding protein (napDNAbp),
(d) a nucleic acid moiety selected from the group consisting of a toe-loop, hairpin, stem-loop, pseudoknot, aptamer, G-quadraplex, tRNA, riboswitch, or ribozyme; and
(e) a linker that links the nucleic acid moiety to another component of the PEgRNA wherein the primer binding site comprises a region of complementarity to a non-target strand of the double-stranded target DNA sequence; wherein the DNA synthesis template comprises a region of complementarity to the non-target strand of the double-stranded target DNA sequence and comprises one or more nucleotide edits compared to the double-stranded target DNA sequence and wherein the linker is designed by a computational model. PEgRNA.
75. The PEgRNA of paragraph 75, wherein the linker comprises a nucleotide sequence selected from the group consisting of SEQ ID NO: 3960, SEQ ID NO: 3961. SEQ ID NO: 3962. SEQ ID NO: 3963, SEQ ID NO: 3964. SEQ ID NO: 3965, SEQ ID NO: 3966. SEQ ID NO: 3967. SEQ ID NO: 3968, SEQ ID NO: 3969. SEQ ID NO: 3970, and SEQ ID NO: 3971.
76. A method for identifying at least one nucleic acid linker for linking a component of a prime editing guide RNA (PEgRNA) to a nucleic acid moiety, the method comprising:
using at least one computer hardware processor to perform:
generating a plurality of nucleic acid linker candidates including a first nucleic acid linker candidate;
identifying the at least one nucleic acid linker from among the plurality of nucleic acid linker candidates at least in part by:
calculating multiple scores for each of at least some of the plurality of nucleic acid linker candidates, the calculating comprising calculating a first set of scores for the first nucleic acid linker candidate, the first set of scores comprising:
a first score indicative of a degree of interaction between the first nucleic acid linker candidate and a first region of the PEgRNA;
a second score indicative of a degree of interaction between the first nucleic acid linker candidate and a second region of the PEgRNA; and
identifying the at least one nucleic acid linker from among the at least some of the plurality of nucleic acid linker candidates using the calculated multiple scores; and outputting information indicative of the at least one nucleic acid linker.
77. The method of paragraph 77, wherein the first score is indicative of a degree to which the first nucleic acid linker candidate is predicted to avoid interaction with the first region of the PEgRNA, and wherein the second score is indicative of a degree to which the first nucleic acid linker candidate is predicted to avoid interaction with the second region of the PEgRNA.
78. The method of paragraph 78, wherein the first region comprises a primer binding site (PBS) of the PEgRNA.
79. The method of paragraph 79, wherein the second region comprises a spacer of the PEgRNA.
80. The method of paragraph 78, wherein the first set of scores further comprises a third score indicative of a degree to which the first nucleic acid linker candidate is predicted to avoid interaction with a third region of the PEgRNA and a fourth score indicative of a degree to which the first nucleic acid linker candidate is predicted to avoid interaction with a fourth region of the PEgRNA.
81. The method of paragraph 81, wherein the third region comprises a DNA synthesis template.
82. The method of paragraph 82, wherein the fourth region comprises a gRNA core that interacts with a nucleic acid programmable DNA binding protein (napDNAbp).
83. The method of paragraph 81, wherein the PEgRNA is for installing a nucleotide edit in a double stranded target DNA sequence,
wherein the PEgRNA comprises:
a spacer sequence that comprises a region of complementarity that hybridizes to a target strand of a double-stranded target DNA sequence,
a nucleic acid extension arm comprising a DNA synthesis template and a primer binding site, and
a gRNA core that interacts with a nucleic acid programmable DNA binding protein napDNAbp.
wherein the primer binding site comprises a region of complementarity to a non-target strand of the double-stranded target DNA sequence;
wherein the DNA synthesis template comprises a region of complementarity to the non-target strand of the double-stranded target DNA sequence and comprises one or more nucleotide edits compared to the double-stranded target DNA sequence and wherein the first region comprises the PBS, the second region comprises the spacer, the third region comprises the DNA synthesis template, and the fourth region comprises the gRNA core.
84. The method of paragraph 77, wherein the plurality of nucleic acid linker candidates comprises a second nucleic acid linker candidate, and wherein identifying the at least one nucleic acid linker from among the at least some of the plurality of nucleic acid linker candidates using the calculated multiple scores comprises: comparing the first set of scores for the first nucleic acid linker candidate with a second set of scores for the second nucleic acid linker candidate.
85. The method of paragraph 85, wherein:
the first region comprises a primer binding site (PBS), the first score in the first set of scores is indicative of a degree to which the first nucleic acid linker candidate is predicted to avoid interaction with the first region of the PEgRNA, a third score in the second set of scores is indicative of a degree to which the second nucleic acid linker candidate is predicted to avoid interaction with the first region of the PEgRNA, and comparing the first set of scores with the second set of scores comprises: comparing the first score with the third score.
86. The method of paragraph 86, wherein when the first score is equal to or is within a threshold distance of the third score, comparing the first set of scores with the second set of scores further comprises: comparing a score, other than the first score, in the first set of scores with another score, other than the third score, in the second set of scores.

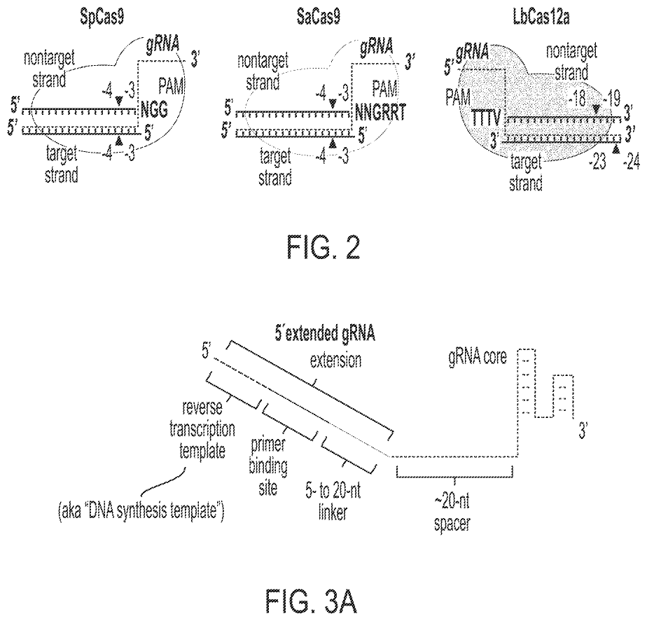

The present disclosure also provides the following numbered embodiments.
1. A method for editing two or more copies of a disease-associated gene, wherein each copy of the disease-associated gene comprises a double stranded target DNA sequence, the method comprising contacting each of the two or more copies of the disease-associated gene with a prime editor system comprising:
(a) a nucleic acid programmable DNA binding protein (napDNAbp) domain or a polynucleotide encoding the napDNAbp domain;
(b) a polymerase domain or a polynucleotide encoding the polymerase domain; and
(c) a prime editing guide RNA (PEgRNA), wherein the PEgRNA comprises:
a spacer that comprises a region of complementarity to a target strand of the double stranded DNA sequence;
a gRNA core that associates with the napDNAbp domain; and
   a nucleic acid extension arm comprising a primer binding site and a DNA synthesis template, wherein the primer binding site comprises a region of complementarity to a non-target strand of the double-stranded target DNA sequence, and wherein the DNA synthesis template comprises a region of complementarity to the non-target strand of the double-stranded target DNA sequence and comprises one or more nucleotide edits compared to the double-stranded target DNA, wherein the non-target strand is complementary to the target strand;
wherein each copy of the disease-associated gene comprises a pathogenic variant and the two or more copies of the disease-associated gene comprise two or more different pathogenic variants, wherein the contacting installs the one or more nucleotide edits in each of the two or more copies of the disease-associated gene, wherein the installation corrects the pathogenic variant in each of the disease-associated genes, thereby editing each of the two or more copies of the disease-associated gene.
2. The method of embodiment 1, wherein the two or more copies of the disease-associated gene are in one subject.
3. The method of embodiment 1, wherein the two or more copies of the disease-associated gene are in two or more different subjects.
4. A method for treating a disease in two or more subjects each comprising a disease-associated gene, wherein the disease-associated gene in of the two or more subjects comprises a double stranded target DNA sequence, the method comprising administering to the two or more subjects a prime editor system comprising:
(a) a nucleic acid programmable DNA binding protein (napDNAbp) domain or a polynucleotide encoding the napDNAbp domain;
(b) a polymerase domain or a polynucleotide encoding the polymerase domain; and
(c) a prime editing guide RNA (PEgRNA), wherein the PEgRNA comprises:
   (i) a spacer that comprises a region of complementarity to a target strand of the double stranded DNA sequence;
   (ii) a gRNA core that associates with the napDNAbp domain; and
   (iii) a nucleic acid extension arm comprising a primer binding site and a DNA synthesis template, wherein the primer binding site comprises a region of complementarity to a non-target strand of the double-stranded target DNA sequence, and wherein the DNA synthesis template comprises a region of complementarity to the non-target strand of the double-stranded target DNA sequence and comprises one or more nucleotide edits compared to the double-stranded target DNA, wherein the non-target strand is complementary to the target strand;
wherein the two or more subjects comprise two or more different pathogenic variants in the disease associated gene, wherein the administration installs the one or more nucleotide edits in the disease associated gene in each of the two or more subjects, wherein the installation corrects the pathogenic variant in the disease-associated gene in each of the two or more subjects, thereby treating the disease in the two or more subjects.
5. The method of any one of embodiments 1-4, wherein the polynucleotide encoding the napDNAbp domain and/or the polynucleotide encoding the polymerase domain comprises RNA, optionally wherein the polynucleotide encoding the napDNAbp domain and/or the polynucleotide encoding the polymerase domain is mRNA.
6. The method of any one of embodiments 1-5, wherein the polymerase domain is an RNA-dependent DNA polymerase domain.
7. The method of embodiment 6, wherein the polymerase domain is a reverse transcriptase, optionally wherein the reverse transcriptase is a reverse transcriptase from a retrovirus or a retrotransposon.
8. The method of embodiment 6, wherein the reverse transcriptase has at least 80%, 85%, 90%, 95%, 98%, or 99% sequence identity with the amino acid sequence of any one of 89-100, 105-122, 128-129, 132, 139, 143, 149, 154, 159, 235, 454, 471, 516, 662, 700, 701-716, 739-741, and 766.
9. The method of embodiment 6, wherein the reverse transcriptase is a Moloney-Murine Leukemia Virus reverse transcriptase (M-MLVRT).
10. The method of embodiment 9, wherein the RNA-dependent DNA polymerase domain comprises a variant Moloney-Murine Leukemia Virus reverse transcriptase (M-MLV RT) domain, wherein the variant M-MLV RT domain comprises one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X relative to the amino acid sequence of SEQ ID NO: 89, and wherein X is any amino acid.
11. The method of embodiment 9, wherein the variant M-MLV RT domain comprises one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N relative to the amino acid sequence of SEQ ID NO: 89.
12. The method of embodiment 11, wherein the variant M-MLV RT domain comprises an amino acid substitutions D200N, T330P and L603W relative to the amino acid sequence of SEQ ID NO: 89.
13. The method of embodiment 11, wherein the M-MLV RT domain comprises amino acid substitutions D200N, T306K, W313F, T330P, and L603W relative to the amino acid sequence of SEQ ID NO: 89.
14. The method of embodiment 11, wherein the variant M-MLV RT domain comprises any one of the amino acid sequence of SEQ ID NOs: 106-122, 143, 701-716, or 740-741.
15. The method of embodiment 11, wherein the M-MLV RT domain has the sequence of SEQ ID NO: 741.
16. The method of embodiment 9, wherein the variant M-MLV RT domain is a truncated variant of M-MLV RT that contains D200N, T306K, W313F, and T330P mutations.
17. The method of embodiment 16, wherein the variant M-MLV RT domain has the sequence of SEQ ID NO: 766.
18. The method of any one of embodiments 1-4, wherein the napDNAbp domain has a nickase activity.
19. The method of any one of embodiments 1-4, wherein the napDNAbp domain is selected from the group consisting of: Cas9, Cas12e, Cas12d, Cas12a, Cas12b1, Cas13a, Cas12c, and Argonaute and optionally has a nickase activity.
20. The method of any one of embodiments 1-4, wherein the napDNAbp domain is a Cas9 protein or variant thereof.
21. The method of embodiment 20, wherein the napDNAbp domain is a nuclease active Cas9, a nuclease inactive Cas9 (dCas9), or a Cas9 nickase (nCas9).
22. The method of embodiment 21, wherein the napDNAbp domain is Cas9 nickase (nCas9).
23. The prime editor of any one of embodiments 1-4, wherein the napDNAbp domain comprises an amino acid sequence having at least 80%, 85%, 90%, 95%, 98%, or 99% sequence identity with the amino acid sequence of any one of SEQ ID NOs: 18, 19, 21, 25, 26, 126, 137, 141, 147, 153, 157, 445, 460, 467, and 482-487.
24. The method of embodiment 23, wherein the napDNAbp domain comprises an amino acid sequence having at least 80%, 85%, 90%, 95%, 98%, or 99% sequence identity with the amino acid sequence to SEQ ID NO: 18.
25. The method of any one of embodiments 1-24, wherein the napDNAbp domain and the RNA-dependent DNA polymerase domain are connected to form a fusion protein.
26. The method of embodiment 25, wherein the napDNAbp domain and the RNA-dependent DNA polymerase domain are connected via a peptide linker to form the fusion protein.
27. The method of embodiment 25 or 26, wherein the fusion protein comprises the structure NH2-[napDNAbp domain]-[RNA-dependent DNA polymerase domain]-COOH, or NH2-[RNA-dependent DNA polymerase domain]-[napDNAbp domain]-COOH, wherein each instance of “]-[” indicates the presence of an optional linker sequence.
28. The method of embodiment 26 or 27, wherein the peptide linker comprises an amino acid sequence selected from SGGS, (2×SGGS), (3×SGGS), XTEN, EAAAK, (2×EAAAK), and (3×EAAAK).
29. The method of embodiment 28, wherein the peptide linker consists of the amino acid sequence of 1×XTEN.
30. The method of embodiment 25, wherein the fusion protein comprises the amino acid sequence of SEQ ID NO:134, or an amino acid sequence having at least 80% sequence identity with the amino acid sequence of SEQ ID NO: 134.
31. The method of any one of embodiments 1-30, wherein the nick site is within a protospacer on the non-target strand of the double stranded target DNA, wherein the protospacer is directly adjacent to a protospacer adjacent motif (PAM).
32. The method of any one of embodiments 1-31, wherein the spacer, the nucleic acid extension arm, and the gRNA core are in a single molecule.
33. The method of embodiment 32, wherein the nucleic acid extension arm is positioned at the 3′ or 5′ end of the gRNA core, or at an intramolecular position in the gRNA core, and optionally wherein the nucleic acid extension arm comprises DNA or RNA.
34. The method of embodiment 32, wherein the nucleic acid extension arm is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides, optionally wherein the nucleic acid extension arm is 10 10 to 20, 20 to 30, 30 to 40, 40 to 50, 50 to 60, 60 to 70, 70 to 80, 80 to 90, 90 to 100, 100 to 110, 110 to 120.20 to 120, 40 to 120, 60 to 120, 80 to 120, 100 to 120, 40 to 100, 60 to 100, 80 to 100, or 60 to 80 nucleotides in length.
35. The method of embodiment 32, wherein the primer binding site is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length, optionally wherein the primer binding site is 1 to 10 nucleotides, 5 to 10 nucleotides, 10 to 15 nucleotides, 10 to 20 nucleotides, 8 to 20 nucleotides, 15 to 25 nucleotides, 20 to 30 nucleotides, or 25 to 30 nucleotides in length.
36. The method of embodiment 35, wherein the primer binding site is from 8 nucleotides to 15 nucleotides in length.
37. The method of embodiment 32, wherein the primer binding site is from (a) 8 nucleotides to 11 nucleotides in length, and contains greater than about 60% GC content, (b) 12 nucleotides to 13 nucleotides in length, and comprises about 40-60% GC content, or (c) 14 nucleotides to 15 nucleotides in length, and contains less than about 40% GC content.
38. The method of embodiment 32, wherein the DNA synthesis template is a reverse transcription template sequence.
39. The method of any one of embodiments 1-38, wherein the DNA synthesis template has a wild type sequence of the disease associated gene.
40. The method of any one of embodiments 1-39, wherein the DNA synthesis template is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length.
41. The method of any one of embodiments 1-39, wherein the DNA synthesis template is 5 to 10, 5 to 15, 10 to 20, 20 to 30, 30 to 40, 40 to 50, 50 to 60, 60 to 70, 70 to 80, 80 to 90, 90 to 100, 10 to 30, 10 to 40, 10 to 50, 10 to 60, 20 to 40, 20 to 60, 30 to 100, 40 to 100, 50 to 100, 60 to 100, or 70 to 100 nucleotides in length, optionally wherein the DNA synthesis template is 10 to 35 nucleotides in length.
42. The method of any one of embodiments 1-39, wherein the DNA synthesis template is at least 3 to 58 nucleotides in length.
43. The method of any one of embodiments 1-39, wherein the DNA synthesis template is from 8 nucleotides to 31 nucleotides in length.
44. The method of any one of embodiments 1-39, wherein the DNA synthesis template is from (a) 10 nucleotides to 16 nucleotides in length or (b) 12 nucleotides to 17 nucleotides in length.
45. The method of embodiment of any one of embodiments 1-39, wherein the DNA synthesis template comprises a nucleotide sequence that is 80%, or 85%, or 90%, or 95%, or 99% identical to the double stranded target DNA sequence.
46. The method of any one of embodiments 1-45, wherein the PEgRNA further comprises at least one nucleic acid moiety selected from the group consisting of a toe-loop, hairpin, stem-loop, pseudoknot, aptamer. G-quadraplex, tRNA, riboswitch, or ribozyme.
47. The method of embodiment 46, wherein the nucleic acid moiety is located at the 3′ or 5′ end of the PEgRNA.
48. The method of embodiment 46, wherein the extension arm comprises the nucleic acid moiety.
49. The method of embodiment 48, wherein the nucleic acid moiety is located at the 3′ or 5′ end of the extension arm.
50. The method of embodiment 46, wherein the nucleic acid moiety comprises a frameshifting pseudoknot from a Moloney murine leukemia virus (M-MLV) genome (a Mpknot), optionally wherein the Mpknot is a Mpknot1 moiety having a nucleotide sequence selected from the group consisting of: SEQ ID NO: 3930 (Mpknot1), SEQ ID NO: 3931 (Mpknot1 3′ trimmed), SEQ ID NO: 3932 (Mpknot1 with 5′ extra). SEQ ID NO: 3933 (Mpknot1 U38A), SEQ ID NO: 3934 (Mpknot1 U38A A29C). SEQ ID NO: 3935 (MMLC A29C), SEQ ID NO: 3936 (Mpknot1 with 5′ extra and U38A). SEQ ID NO: 3937 (Mpknot1 with 5′ extra and U38A A29C), and SEQ ID NO: 3938 (Mpknot1 with 5′ extra and A29C), or a nucleotide sequence having at least 80% sequence identity therewith.
51. The method of embodiment 46, wherein the nucleic acid moiety comprises a G-quadruplex, optionally wherein the G-quadruplex has a nucleotide sequence selected from the group consisting of: SEQ ID NO: 3939 (tns1), SEQ ID NO: 3940 (stk40), SEQ ID NO: 3941 (apc2), SEQ ID NO: 3942 (ceacam4), SEQ ID NO: 3943 (pitpnm3), SEQ ID NO: 3944 (rlf), SEQ ID NO: 3945 (erc1). SEQ ID NO: 3946 (ube3c). SEQ ID NO: 3947 (taf15). SEQ ID NO: 3948 (stard3), and SEQ ID NO: 3949 (g2), or a nucleotide sequence having at least 80% sequence identity therewith.
52. The method of embodiment 46, wherein the nucleic acid moiety comprises a prequeosine 1 riboswitch aptamer, optionally wherein the nucleic acid moiety comprises an evolved prequeosine1-1 riboswitch aptamer (evopreQ1 comprising a nucleotide sequence selected from the group consisting of: SEQ ID NO: 3950 (evopreq1). SEQ ID NO: 3951 (evopreq1motif1), SEQ ID NO: 3952 (evopreq1motif2), SEQ ID NO: 3953 (evopreq1motif3), SEQ ID NO: 3954 (shorter preg1-1), SEQ ID NO: 3955 (preq1-1 G5C (mut1)), and SEQ ID NO: 3956 (preq1-1 G15C (mut2)), or a nucleotide sequence having at least 80% sequence identity therewith.
53. The method of embodiment 46, wherein the nucleic acid moiety comprises a tRNA moiety having a nucleotide sequence of SEQ ID NO: 3957, or a nucleotide sequence having at least 80% sequence identity therewith.
54. The method of embodiment 46, wherein the nucleic acid moiety has a nucleotide sequence of SEQ ID NO: 3958 (xrn1), or a nucleotide sequence having at least 80% sequence identity therewith.
55. The method of embodiment 46, wherein the nucleic acid moiety comprises a P4-P6 domain of a group I intron, optionally wherein the P4-P6 domain has a nucleotide sequence of SEQ ID NO: 3959, or a nucleotide sequence having at least 80% sequence identity therewith.
56. The method of any of embodiments 46-55, wherein the PEgRNA further comprises a linker.
57. The method of embodiment 56, wherein the linker is between the nucleic acid moiety and another component of the PEgRNA.
58. The method of embodiment 57, wherein the linker is between the nucleic acid moiety and the primer binding site or between the gRNA core and the nucleic acid moiety.
59. The method of embodiment 58, wherein the linker comprises a nucleotide sequence selected from the group consisting of SEQ ID NO: 3960, SEQ ID NO: 3961. SEQ ID NO: 3962, SEQ ID NO: 3963, SEQ ID NO: 3964. SEQ ID NO: 3965, SEQ ID NO: 3966. SEQ ID NO: 3967. SEQ ID NO: 3968, SEQ ID NO: 3969. SEQ ID NO: 3970, and SEQ ID NO: 3971.
60. The method of any one of embodiments 1-59, wherein the one or more nucleotide edits comprises an insertion of one or more nucleotides as compared to the double-stranded DNA sequence.
61. The method of any one of embodiments 1-59, wherein the one or more nucleotide edits comprises a deletion of one or more nucleotides as compared to the double-stranded DNA sequence.
62. The method of any one of embodiments 1-59, wherein the one or more nucleotide edits comprises a nucleotide substitution as compared to the double-stranded DNA sequence.
63. The method of any one of embodiments 1-59, wherein the wherein the one or more nucleotide edits comprises one or more insertions of one or more nucleotides, nucleotide substitutions, deletions of one or more nucleotides, or a combination of any such nucleotide edits as compared to the double-stranded target DNA sequence.
64. The method of any one of embodiments 62-63, wherein the one or more nucleotide substitutions are single-base nucleotide substitutions.
65. The method of any one of embodiments 2-65, wherein the administration corrects 2, 3, 4, 5, 6, 7, 8, 9, 10, 11, 12, 13, 14, 15, 16, 17, 18, 19 or 20 pathogenic variants in the disease-associated gene in the two or more subjects; or wherein the administration corrects 2 to 5, 2 to 7, 3 to 10, 3 to 12, 4 to 15, or 4 to 20 pathogenic variants in the disease-associated gene in the two or more subjects.
66. The method of any one of embodiments 1-63, wherein the PEgRNA comprises a modified nucleobase, a modified sugar, a modified phosphate group, or a nucleoside analog.
67. The method of embodiment 4-64, comprising administering to the two or more subjects a pharmaceutical composition comprising the method and a pharmaceutically acceptable excipient.
68. The method of any one of embodiments 1-65, wherein the disease associated gene is CDKL5.
69. The method of embodiment 66, wherein each of the different pathogenic variants encodes a mutation selected from the group consisting of V1721, A173D, R175S, W176G, W176R, Y177C, R178P, P180L, E181A, and L182P as compared to a wild type CDKL5 protein.
70. The method of embodiment 66, wherein the PEgRNA comprises the sequence of PEgRNA sequence in FIG. 2.
71. The method of embodiment 66, wherein the PEgRNA comprises the sequence of PEgRNA sequence in FIG. 4.

### EXAMPLES

### Example 1: Prime Editing: Highly Versatile and Precise Search-and-Replace Genome Editing in Human Cells without Double-Stranded DNA Breaks

### Background

Current genome editing methods can disrupt, delete, or insert target genes with accompanying byproducts of double-stranded DNA breaks using programmable nucleases, and install the four transition point mutations at target loci using base editors. Small insertions, small deletions, and the eight transversion point mutations, however, collectively represent most pathogenic genetic variants but cannot be corrected efficiently and without an excess of byproducts in most cell types. Described herein is prime editing, a highly versatile and precise genome editing method that directly writes new genetic information into a specified DNA site using a catalytically impaired Cas9 fused to an engineered reverse transcriptase, programmed with an engineered prime editing guide RNA (pegRNA) that both specifies the target site and encodes the desired edit. Greater than 175 distinct edits in human cells were performed to establish that prime editing can make targeted insertions, deletions, all 12 possible types of point mutations, and combinations thereof efficiently (typically 20-60%, up to 77% in unsorted cells) and with low byproducts (typically 1-10%), without requiring double-stranded breaks or donor DNA templates. Prime editing was applied in human cells to correct the primary genetic causes of sickle cell disease (requiring an A•T-to-T•A transversion in HBB) and Tay-Sachs disease (requiring a 4-base deletion in HEXA), in both cases efficiently reverting the pathogenic genomic alleles to wild-type with minimal byproducts. Prime editing was also used to create human cell lines with these pathogenic HBB transversion and HEXA insertion mutations, to install the G127V mutation in PRNP that confers resistance to prion disease (requiring a G•C-to-T•A transversion), and to efficiently insert a His6 tag, a FLAG epitope tag, and an extended LoxP site into target loci in human cells. Prime editing offers efficiency and product purity advantages over HDR, and complementary strengths and weaknesses compared to base editing. Consistent with its search-and-replace mechanism, which requires three distinct base-pairing events, prime editing is much less prone to off-target DNA modification at known Cas9 off-target sites than Cas9. Prime editing substantially expands the scope and capabilities of genome editing, and in principle can correct ˜89% of known pathogenic human genetic variants.

The ability to make virtually any targeted change in the genome of any living cell or organism is a longstanding aspiration of the life sciences. Despite rapid advances in genome editing technologies, the majority of the >75,000 known human genetic variants associated with diseases¹¹¹ cannot be corrected or installed in most therapeutically relevant cells (FIG. 38A). Programmable nucleases such as CRISPR-Cas9 make double-stranded DNA breaks (DSBs) that can disrupt genes by inducing mixtures of insertions and deletions (indels) at target sites¹¹²⁻¹¹⁴. Nucleases can also be used to delete target genes^{115,116}, or insert exogenous genes¹¹⁷⁻¹¹⁹, through homology-independent processes. Double-stranded DNA breaks, however, are also associated with undesired outcomes including complex mixtures of products, translocations¹²⁰, and p53 activation^{121,122}. Moreover, the vast majority of pathogenic alleles differ from their non-pathogenic counterparts by small insertions, deletions, or base substitutions that require much more precise editing technologies to correct (FIG. 38A). Homology-directed repair (HDR) stimulated by nuclease-induced DSBs¹²³ has been widely used to install a variety of precise DNA changes. HDR, however, relies on exogenous donor DNA repair templates, typically generates an excess of indel byproducts from end-joining repair of DSBs, and is inefficient in most therapeutically relevant cell types (T cells and some stem cells being important exceptions)^{124,125}. While enhancing the efficiency and precision of DSB-mediated genome editing remains the focus of promising efforts¹²⁶⁻¹³⁰, these challenges necessitate the exploration of alternative precision genome editing strategies.

Base editing can efficiently install or correct the four types of transition mutations (C to T, G to A, A to G, and T to C) without requiring DSBs in a wide variety of cell types and organisms, including mammals¹²⁸⁻¹³¹, but cannot currently achieve any of the eight transversion mutations (C to A, C to G, G to C, G to T, A to C, A to T, T to A, and T to G), such as the T•A-to-A•T mutation needed to directly correct the most common cause of sickle cell disease (HBB E6V)¹³². In addition, no DSB-free method has been reported to perform target deletions, such as the removal of the 4-base duplication that causes Tay-Sachs disease (HEXA 1278+TATC)¹³, or targeted insertions, such as the precise 3-base insertion required to directly correct the most common cause of cystic fibrosis (CFTR AF508)¹³⁴. Targeted transversion point mutations, insertions, and deletions thus are difficult to install or correct efficiently and without excess byproducts in most cell types, even though they collectively account for most known pathogenic alleles (FIG. 38A).

Described herein is the development of prime editing, a new “search-and-replace” genome editing technology that mediates targeted insertions, deletions, and all 12 possible base-to-base conversions at targeted loci in human cells without requiring double-stranded DNA breaks, or donor DNA templates. Prime editors, initially exemplified by PE1, use a reverse transcriptase fused to a programmable nickase and a prime editing pegRNA (pegRNA) to directly copy genetic information from the extension on the pegRNA into the target genomic locus. A second-generation prime editor (PE2) uses an engineered reverse transcriptase to substantially increase editing efficiencies with minimal (typically <2%) indel formation, while a third-generation PE3 system adds a second guide RNA to nick the non-edited strand, thereby favoring replacement of the non-edited strand and further increasing editing efficiency, typically, to about 20-50% in human cells with about 1-10% indel formation. PE3 offers far fewer byproducts and higher or similar efficiency compared to optimized Cas9 nuclease-initiated HDR, and offers complementary strengths and weaknesses compared to current-generation base editors.

PE3 was applied at genomic loci in human HEK293T cells to achieve efficient conversion of HBB E6V to wild-type HBB, deletion of the inserted TATC to restore HEXA 1278+TATC to wild-type HEXA, installation in PRNP of the G127V mutation that confers resistance to prion disease¹³⁵ (requiring a G•C-to-T•A transversion), and targeted insertion of a His6 tag (18 bp), FLAG epitope tag (24 bp), and extended LoxP site for Cre-mediated recombination (44 bp). Prime editing was also successful in three other human cell lines, as well as in post-mitotic primary mouse cortical neurons, with varying efficiencies. Due to a high degree of flexibility in the distance between the initial nick and location of the edit, prime editing is not substantially constrained by the PAM requirement of Cas9 and in principle can target the vast majority of genomic loci. Off-target prime editing is much rarer than off-target Cas9 editing at known Cas9 off-target loci, likely due to the requirement of three distinct DNA base pairing events in order for productive prime editing to take place. By enabling precise targeted insertions, deletions, and all 12 possible classes of point mutations at a wide variety of genomic loci without the need for DSBs or donor DNA templates, prime editing has the potential to advance the study and correction of many gene variants.

### Results

Strategy for Transferring Information from an pegRNA into a Target DNA Locus

Cas9 targets DNA using a guide RNA containing a spacer sequence that hybridizes to the target DNA site^{112-114,136,137}. The aim was to engineer guide RNAs to both specify the DNA target as in natural CRISPR systems^{138,139}, and also to contain new genetic information that replaces the corresponding DNA nucleotides at the target locus. The direct transfer of genetic information from an pegRNA into a specified DNA site, followed by replacement of the original unedited DNA, in principle could provide a general means of installing targeted DNA sequence changes in living cells, without dependence on DSBs or donor DNA templates. To achieve this direct information transfer, the aim was to use genomic DNA, nicked at the target site to expose a 3′-hydroxyl group, to prime the reverse transcription of the genetic information from an extension on the engineered guide RNA (hereafter referred to as the prime editing guide RNA, or pegRNA) directly into the target site (FIG. 38A).

These initial steps of nicking and reverse transcription, which resemble mechanisms used by some natural mobile genetic elements¹⁴⁰ result in a branched intermediate with two redundant single-stranded DNA flaps on one strand: a 5′ flap that contains the unedited DNA sequence, and a 3′ flap that contains the edited sequence copied from the pegRNA (FIG. 38B). To achieve a successful edit, this branched intermediate must be resolved so that the edited 3′ flap replaces the unedited 5′ flap. While hybridization of the 5′ flap with the unedited strand is likely to be thermodynamically favored since the edited 3′ flap can make fewer base pairs with the unedited strand, 5′ flaps are the preferred substrate for structure-specific endonucleases such as FEN¹¹⁴¹ which excises 5′ flaps generated during lagging-strand DNA synthesis and long-patch base excision repair. It was reasoned that preferential 5′ flap excision and 3′ flap ligation could drive the incorporation of the edited DNA strand, creating heteroduplex DNA containing one edited strand and one unedited strand (FIG. 38B).

Permanent installation of the edit could arise from subsequent DNA repair that resolves the mismatch between the two DNA strands in a manner that copies the information in the edited strand to the complementary DNA strand (FIG. 38C). Based on a similar strategy developed to maximize the efficiency of DNA base editing¹³¹⁻¹³³ it was envisioned that nicking the non-edited DNA strand, far enough from the site of the initial nick to minimize double-strand break formation, might bias DNA repair to preferentially replace the non-edited strand.

### Validation of Prime Editing Steps In Vitro and in Yeast Cells

Following cleavage of the PAM-containing DNA strand by the RuvC nuclease domain of Cas9, the PAM-distal fragment of this strand can dissociate from otherwise stable Cas9:sgRNA:DNA complexes¹⁴³. The 3′ end of this liberated strand can be sufficiently accessible to prime DNA polymerization. Guide RNA engineering efforts¹⁴⁴⁻¹⁴⁶ and crystal structures of Cas9:sgRNA:DNA complexes¹⁴⁷⁻¹⁴⁹ suggest that the 5′ and 3′ termini of the sgRNA can be extended without abolishing Cas9:sgRNA activity. pegRNAs were designed by extending sgRNAs to include two critical components: a primer binding site (PBS) that allows the 3′ end of the nicked DNA strand to hybridize to the pegRNA, and a reverse transcriptase (RT) template containing the desired edit that would be directly copied into the genomic DNA site as the 3′ end of the nicked DNA strand is extended across the RNA template by a polymerase (FIG. 38C).

These hypotheses were tested in vitro using purified S. pyogenes Cas9 protein. A series of pegRNA candidates were constructed by extending sgRNAs on either terminus with a PBS sequence (5 to 6 nucleotides, nt) and an RT template (7 to 22 nt). It was confirmed that 5′-extended pegRNAs direct Cas9 binding to target DNA, and that both 5′-extended pegRNAs and 3′-extended pegRNAs support Cas9-mediated target nicking in vitro and DNA cleavage activities in mammalian cells (FIGS. 44A-44C). These candidate pegRNA designs were tested using pre-nicked 5′-Cy5-labeled dsDNA substrates, catalytically dead Cas9 (dCas9), and a commercial variant of Moloney murine leukemia virus (M-MLV) reverse transcriptase (FIG. 44D). When all components were present, efficient conversion of the fluorescently labeled DNA strand into longer DNA products with gel mobilities, consistent with reverse transcription along the RT template, (FIG. 38D, FIGS. 44D-44E) was observed. Products of desired length were formed with either 5′-extended or 3′-extended pegRNAs (FIGS. 38D-38E). Omission of dCas9 led to nick translation products derived from reverse transcriptase-mediated DNA polymerization on the DNA template, with no pegRNA information transfer (FIG. 38D). No DNA polymerization products were observed when the pegRNA was replaced by a conventional sgRNA, confirming the necessity of the PBS and RT template components of the pegRNA (FIG. 38D). These results demonstrate that Cas9-mediated DNA melting exposes a single-stranded R-loop that, if nicked, is competent to prime reverse transcription from either a 5′-extended or 3′-extended pegRNA.

Next, non-nicked dsDNA substrates were tested with a Cas9 nickase (H840A mutant) that exclusively nicks the PAM-containing strand¹¹². In these reactions, 5′-extended pegRNAs generated reverse transcription products inefficiently, possibly due to impaired Cas9 nickase activity (FIG. 44F). However, 3′-extended pegRNAs enabled robust Cas9 nicking and efficient reverse transcription (FIG. 38E). The use of 3′-extended pegRNAs generated only a single apparent product, despite the potential, in principle, for reverse transcription to terminate anywhere within the remainder of the pegRNA. DNA sequencing of the products of reactions with Cas9 nickase, RT, and 3′-extended pegRNAs revealed that the complete RT template sequence was reverse transcribed into the DNA substrate (FIG. 44G). These experiments established that 3′-extended pegRNAs can template the reverse transcription of new DNA strands while retaining the ability to direct Cas9 nickase activity.

To evaluate the eukaryotic cell DNA repair outcomes of 3′ flaps produced by pegRNA-programmed reverse transcription in vitro, DNA nicking and reverse transcription using pegRNAs, Cas9 nickase, and RT in vitro on reporter plasmid substrates were performed, and the reaction products were then transformed into yeast (S. cerevisiae) cells (FIG. 45A). Encouragingly, when plasmids were edited in vitro with 3′-extended pegRNAs encoding a T•A-to-A•T transversion that corrects the premature stop codon, 37% of yeast transformants expressed both GFP and mCherry proteins (FIG. 38F, FIG. 45C). Consistent with the results in FIG. 38E and FIG. 44F, editing reactions carried out in vitro with 5′-extended pegRNAs yielded fewer GFP and mCherry double-positive colonies (9%) than those with 3′-extended pegRNAs (FIG. 38F and FIG. 45D). Productive editing was also observed using 3′-extended pegRNAs that insert a single nucleotide (15% double-positive transformants) or delete a single nucleotide (29% double-positive transformants) to correct frameshift mutations (FIG. 38F and FIGS. 45E-45F). DNA sequencing of edited plasmids recovered from double-positive yeast colonies confirmed that the encoded transversion edit occurred at the desired sequence position (FIG. 45G). These results demonstrate that DNA repair in eukaryotic cells can resolve 3′ DNA flaps arising from prime editing to incorporate precise DNA edits including transversions, insertions, and deletions.

### Design of Prime Editor 1 (PE1)

Encouraged by the results in vitro and in yeast, a prime editing system with a minimum number of components capable of editing genomic DNA in mammalian cells was sought for development. 3′-extended pegRNAs (hereafter referred to simply as pegRNAs, FIG. 39A) and direct fusions of Cas9 H840A to reverse transcriptase via a flexible linker can constitute a functional two-component prime editing system. HEK293T (immortalized human embryonic kidney) cells were transfected with one plasmid encoding a fusion of wild-type M-MLV reverse transcriptase to either terminus of Cas9 H840A nickase as well as a second plasmid encoding a pegRNA. Initial attempts led to no detectable T•A-to-A•T conversion at the HEK3 target locus.

Extension of the PBS in the pegRNA to 8-15 bases (FIG. 39A), however, led to detectable T•A-to-A•T editing at the HEK3 target site (FIG. 39B), with higher efficiencies for prime editor constructs in which the RT was fused to the C-terminus of Cas9 nickase (3.7% maximal T•A-to-A•T conversion with PBS lengths ranging from 8-15 nt) compared to N-terminal RT-Cas9 nickase fusions (1.3% maximal T•A-to-A•T conversion) (FIG. 39B; all mammalian cell data herein reports values for the entire treated cell population, without selection or sorting, unless otherwise specified). These results suggest that wild-type M-MLV RT fused to Cas9 requires longer PBS sequences for genome editing in human cells compared to what is required in vitro using the commercial variant of M-MLV RT supplied in trans. This first-generation wild-type M-MLV reverse transcriptase fused to the C-terminus of Cas9 H840A nickase was designated as PE1.

The ability of PE1 to precisely introduce transversion point mutations at four additional genomic target sites specified by the pegRNA (FIG. 39C) was tested. Similar to editing at the HEK3 locus, efficiency at these genomic sites was dependent on PBS length, with maximal editing efficiencies ranging from 0.7-5.5% (FIG. 39C). Indels from PE1 were low, averaging 0.2±0.1% for the five sites under conditions that maximized each site's editing efficiency (FIG. 46A). PE1 was also able to install targeted insertions and deletions, exemplified by a single-nucleotide deletion (4.0% efficiency), a single-nucleotide insertion (9.7%), and a three-nucleotide insertion (17%) at the HEK3 locus (FIG. 39C). These results establish the ability of PE1 to directly install targeted transversions, insertions, and deletions without requiring double-stranded DNA breaks or DNA templates.

### Design of Prime Editor 2 (PE2)

While PE1 can install a variety of edits at several loci in HEK293T cells, editing efficiencies were generally low (typically ≤5%) (FIG. 39C). Engineering the reverse transcriptase in PE1 might improve the efficiency of DNA synthesis within the unique conformational constraints of the prime editing complex, resulting in higher genome editing yields. M-MLV RT mutations have been previously reported that increase enzyme thermostability^{150,151}, processivity¹⁵⁰, and DNA:RNA heteroduplex substrate affinity¹⁵², and that inactivate RNaseH activity¹⁵³. 19 PE1 variants were constructed containing a variety of reverse transcriptase mutations to evaluate their prime editing efficiency in human cells.

First, a series of M-MLV RT variants that previously emerged from laboratory evolution for their ability to support reverse transcription at elevated temperatures¹⁵⁰ were investigated. Successive introduction of three of these amino acid substitutions (D200N, L603W, and T330P) into M-MLV RT, hereafter referred to as M3, led to a 6.8-fold average increase in transversion and insertion editing efficiency across five genomic loci in HEK293T cells compared to that of PE1 (FIGS. 47A-47S).

Next, in combination with M3, additional reverse transcriptase mutations that were previously shown to enhance binding to template:PBS complex, enzyme processivity, and thermostability¹⁵² were tested. Among the 14 additional mutants analyzed, a variant with T306K and W313F substitutions, in addition to the M3 mutations, improved editing efficiency an additional 1.3-fold to 3.0-fold compared to M3 for six transversion or insertion edits across five genomic sites in human cells (FIGS. 47A-47S). This pentamutant of M-MLV reverse transcriptase incorporated into the PE1 architecture (Cas9 H840A-M-MLV RT (D200N L603W T330P T306K W313F)) is hereafter referred to as PE2.

PE2 installs single-nucleotide transversion, insertion, and deletion mutations with substantially higher efficiency than PE1 (FIG. 39C), and is compatible with shorter PBS pegRNA sequences (FIG. 39C), consistent with an enhanced ability to productively engage transient genomic DNA:PBS complexes. On average, PE2 led to a 1.6- to 5.1-fold improvement in prime editing point mutation efficiency over PE1 (FIG. 39C), and in some cases dramatically improved editing yields up to 46-fold (FIG. 47F and FIG. 47I). PE2 also effected targeted insertions and deletions more efficiently than PE1, achieving the targeted insertion of the 24-bp FLAG epitope tag at the HEK3 locus with 4.5% efficiency, a 15-fold improvement over the efficiency of installing this insertion with PE1 (FIG. 47D), and mediated a 1-bp deletion in HEK3 with 8.6% efficiency, 2.1-fold higher than that of PE1 (FIG. 39C). These results establish PE2 as a more efficient prime editor than PE1.

Optimization of pegRNA Features

The relationship between pegRNA architecture and prime editing efficiency was systematically probed at five genomic loci in HEK293T cells with PE2 (FIG. 39C). In general, priming sites with lower GC content required longer PBS sequences (EMX1 and RNF2, containing 40% and 30% GC content, respectively, in the first 10 nt upstream of the nick), whereas those with greater GC content supported prime editing with shorter PBS sequences (HEK4 and FANCF, containing 80% and 60% GC content, respectively, in the first 10 nt upstream of the nick) (FIG. 39C), consistent with the energetic requirements for hybridization of the nicked DNA strand to the pegRNA PBS. No PBS length or GC content level was strictly predictive of prime editing efficiency, and other factors such as secondary structure in the DNA primer or pegRNA extension may also influence editing activity. It is recommended to start with a PBS length of ˜13 nt for a typical target sequence, and exploring different PBS lengths if the sequence deviates from −40-60% GC content. When necessary, optimal PBS sequences should be determined empirically.

Next, the performance determinants of the RT template portion of the pegRNA were studied. pegRNAs with RT templates ranging from 10-20 nt in length were systemically evaluated at five genomic target sites using PE2 (FIG. 39D) and with longer RT templates as long as 31 nt at three genomic sites (FIGS. 48A-48C). As with PBS length, RT template length also could be varied to maximize prime editing efficiency, although in general many RT template lengths ≥10 nt long support more efficient prime editing (FIG. 39D). Since some target sites preferred longer RT templates (>15 nt) to achieve higher editing efficiencies (FANCF, EMX1), while other loci preferred short RT templates (HEK3, HEK4) (FIG. 39D), it is recommend both short and long RT templates be tested when optimizing a pegRNA, starting with ˜10-16 nt.

Importantly, RT templates that place a C as the nucleotide adjacent to the terminal hairpin of the sgRNA scaffold generally resulted in lower editing efficiency compared to other pegRNAs with RT templates of similar length (FIGS. 48A-48C). Based on the structure of sgRNAs bound to Cas9^{148,149}, it was considered that the presence of a C as the first nucleotide of the 3′ extension of a canonical sgRNA can disrupt the sgRNA scaffold fold by pairing with G81, a nucleotide that natively forms a pi stack with Tyr 1356 in Cas9 and a non-canonical base pair with sgRNA A68. Since many RT template lengths support prime editing, it is recommended to choose pegRNAs in which the first base of the 3′ extension (the last reverse-transcribed base of the RT template) is not C.

### Design of Prime Editor 3 Systems (PE3 and PE3b)

While PE2 can transfer genetic information from the pegRNA to the target locus more efficiently than PE1, the manner in which the cell resolves the resulting heteroduplex DNA created by one edited strand and one unedited strand determines if the edit is durable. A previous development of base editing faced a similar challenge since the initial product of cytosine or adenine deamination is heteroduplex DNA containing one edited and one non-edited strand. To increase the efficiency of base editing, a Cas9 D10A nickase was used to introduce a nick into the non-edited strand and to direct DNA repair to that strand, using the edited strand as a template^{129,130,142}. To exploit this principle to enhance prime editing efficiencies, a similar strategy of nicking the non-edited strand using the Cas9 H840A nickase already present in PE2 and a simple sgRNA to induce preferential replacement of the non-edited strand by the cell (FIG. 40A) was tested. Since the edited DNA strand was also nicked to initiate prime editing, a variety of sgRNA-programmed nick locations were tested on the non-edited strand to minimize the production of double-stranded DNA breaks that lead to indels.

This PE3 strategy was first tested at five genomic sites in HEK293T cells by screening sgRNAs that induce nicks located 14 to 116 bases from the site of the pegRNA-induced nick, either 5′ or 3′ of the PAM. In four of the five sites tested, nicking the non-edited strand increased the amount of indel-free prime editing products compared to the PE2 system by 1.5- to 4.2-fold, to as high as 55% (FIG. 40B). While the optimal nicking position varied depending on the genomic site, nicks positioned 3′ of the PAM (positive distances in FIG. 40B) approximately 40-90 bp from the pegRNA-induced nick generally produced favorable increases in prime editing efficiency (averaging 41%) without excess indel formation (6.8% average indels for the sgRNA resulting in the highest editing efficiency for each of the five sites tested) (FIG. 40B). As expected, at some sites, placement of the non-edited strand nick within 40 bp of the pegRNA-induced nick led to large increases in indel formation up to 22% (FIG. 40B), presumably due to the formation of a double-strand break from nicking both strands close together. At other sites, however, nicking as close as 14 bp away from the pegRNA-induced nick produced only 5% indels (FIG. 40B), suggesting that locus-dependent factors control conversion of proximal dual nicks into double-strand DNA breaks. At one tested site (HEK4), complementary strand nicks either provided no benefit or led to indel levels that surpassed editing efficiency (up to 26%), even when placed at distances >70 bp from the pegRNA-induced nick, consistent with an unusual propensity of the edited strand at that site to be nicked by the cell, or to be ligated inefficiently. It is recommend to start with non-edited strand nicks approximately 50 bp from the pegRNA-mediated nick, and to test alternative nick locations if indel frequencies exceed acceptable levels.

This model for how complementary strand nicking improved prime editing efficiency (FIG. 40A) predicted that nicking the non-edited strand only after edited strand flap resolution could minimize the presence of concurrent nicks, decreasing the frequency of double-strand breaks that go on to form indels. To achieve temporal control over non-edited strand nicking, sgRNAs with spacer sequences that match the edited strand, but not the original allele, were designed. Using this strategy, referred to hereafter as PE3b, mismatches between the spacer and the unedited allele should disfavor nicking by the sgRNA until after the editing event on the PAM strand takes place. This PE3b approach was tested with five different edits at three genomic sites in HEK293T cells and compared outcomes to those achieved with PE2 and PE3 systems. In all cases, PE3b was associated with substantially lower levels of indels compared to PE3 (3.5- to 30-fold, averaging 12-fold lower indels, or 0.85%), without any evident decrease in overall editing efficiency compared to PE3 (FIG. 40C). Therefore, when the edit lay within a second protospacer, the PE3b system could decrease indels while still improving editing efficiency compared to PE2, often to levels similar to those of PE3 (FIG. 40C).

Together, these findings established that PE3 systems (Cas9 nickase-optimized reverse transcriptase+pegRNA+sgRNA) improved editing efficiencies ˜3-fold compared with PE2 (FIGS. 40B-40C). PE3 was accompanied by wider ranges of indels than PE2, as expected given the additional nicking activity of PE3. The use of PE3 is recommended when prioritizing prime editing efficiency. When minimization of indels is critical, PE2 offers ˜10-fold lower indel frequencies. When it is possible to use a sgRNA that recognizes the installed edit to nick the non-edited strand, the PE3b system can achieve PE3-like editing levels while greatly reducing indel formation.

To demonstrate the targeting scope and versatility of prime editing with PE3, the installation of all possible single nucleotide substitutions across the +1 to +8 positions (counting the first base 3′ of the pegRNA-induced nick as position +1) of the HEK3 target site using PE3 and pegRNAs with 10-nucleotide RT templates (FIG. 41A) was explored. Collectively, these 24 distinct edits cover all four transition mutations and all eight transversion mutations, and proceed with editing efficiencies (containing no indels) averaging 33±7.9% (ranging between 14% and 48%), with an average of 7.5±1.8% indels.

Importantly, long-distance RT templates could also give rise to efficient prime editing with PE3. For example, using PE3 with a 34-nt RT template, point mutations were installed at positions +12, +14, +17, +20, +23, +24, +26, +30, and +33 (12 to 33 bases from the pegRNA-induced nick) in the HEK3 locus with an average of 36±8.7% efficiency and 8.6±2.0% indels (FIG. 41B). Although edits beyond the +10 position at other loci were not attempted, other RT templates ≥30 nt at three alternative sites also support efficient editing (FIGS. 48A-C). The viability of long RT templates enabled efficient prime editing for dozens of nucleotides from the initial nick site. Since an NGG PAM on either DNA strand occurs on average every ˜8 bp, far less than maximum distances between the edit and the PAM that support efficient prime editing, prime editing is not substantially constrained by the availability of a nearby PAM sequence, in contrast with other precision genome editing methods^{125,142,154}. Given the presumed relationship between RNA secondary structure and prime editing efficiency, when designing pegRNAs for long-range edits it is prudent to test RT templates of various lengths and, if necessary, sequence compositions (e.g., synonymous codons) to optimize editing efficiency.

To further test the scope and limitations of the PE3 system for introducing transition and transversion point mutations, 72 additional edits covering all 12 possible types of point mutations across six additional genomic target sites (FIG. 41C-41H) were tested. Overall, indel-free editing efficiency averaged 25±14%, while indel formation averaged 8.3±7.5%. Since the pegRNA RT template included the PAM sequence, prime editing could induce changes to the PAM sequence. In these cases, higher editing efficiency (averaging 39±9.7%) and lower indel generation (averaging 5.0±2.9%) were observed (FIGS. 41A-41K, point mutations at positions +5 or +6). This increase in efficiency and decrease in indel formation for PAM edits may arise from the inability of the Cas9 nickase to re-bind and nick the edited strand prior to the repair of the complementary strand. Since prime editing supports combination edits with no apparent loss of editing efficiency, editing the PAM, in addition to other desired changes, when possible, is recommended.

Next, 14 targeted small insertions and 14 targeted small deletions at seven genomic sites using PE3 (FIG. 41I) were performed. Targeted 1-bp insertions proceeded with an average efficiency of 32±9.8%, while 3-bp insertions were installed with an average efficiency of 39±16%. Targeted 1-bp and 3-bp deletions were also efficient, proceeding with an average yield of 29±14% and 32±11%, respectively. Indel generation (beyond the targeted insertion or deletion) averaged 6.8±5.4%. Since insertions and deletions introduced between positions +1 and +6 alter the position or the structure of the PAM, it was considered that insertion and deletion edits in this range are typically more efficient due to the inability of Cas9 nickase to re-bind and nick the edited DNA strand prior to repair of the complementary strand, similar to point mutations that edit the PAM.

PE3 was also tested for its ability to mediate larger precise deletions of 5 bp to 80 bp at the HEK3 site (FIG. 41J). Very high editing efficiencies (52 to 78%) were observed for 5-, 10-, and 15-bp deletions when using a 13-nt PBS and an RT template that contained 29, 24, or 19 bp of homology to the target locus, respectively. Using a 26-nt RT template supported a larger deletion of 25 bp with 72±4.2% efficiency, while a 20-nt RT template enabled an 80-bp deletion with an efficiency of 52±3.8%. These targeted deletions were accompanied by indel frequencies averaging 11±4.8% (FIG. 41J).

Finally, the ability of PE3 to mediate 12 combinations of multiple edits at the same target locus consisting of insertions and deletions, insertions and point mutations, deletions and point mutations, or two point mutations across three genomic sites was tested. These combination edits were very efficient, averaging 55% of the target edit with 6.4% indels (FIG. 41K), and demonstrating the ability of prime editing to make combinations of precision insertions, deletions, and point mutations at individual target sites with high efficiency and low indel frequencies.

Together, the examples in FIGS. 41A-41K represent 156 distinct transition, transversion, insertion, deletion, and combination edits across seven human genomic loci. These findings establish the versatility, precision, and targeting flexibility of prime editing.

Prime Editing Compared with Base Editing

Current-generation cytidine base editors (CBEs) and adenine base editors (ABEs) can install C•G-to-T•A transition mutations and A•T-to-G•C transition mutations with high efficiency and low indels^{129,130,142}. The application of base editing can be limited by the presence of multiple cytidine or adenine bases within the base editing activity window (typically ˜5-bp wide), which gives rise to unwanted bystander edits^{129,130,142,155}, or by the absence of a PAM positioned approximately 15±2 nt from the target nucleotide^{142,156} Prime editing can be particularly useful for precise installation of transitions mutations without bystander edits, or when the lack of suitably positioned PAMs precludes favorable positioning the target nucleotide within the CBE or ABE activity window.

Prime editing and cytosine base editing was compared by editing three genomic loci that contain multiple target cytidines in the canonical base editing window (protospacer positions 4-8, counting the PAM as positions 21-23) using optimized CBEs¹⁵⁷ without nickase activity (BE2max) or with nickase activity (BE4max), or using the analogous PE2 and PE3 prime editing systems. Among the nine total target cytosines within the base editing windows of the three sites, BE4max yielded 2.2-fold higher average total C•G-to-T•A conversion than PE3 for bases in the center of the base editing window (protospacer positions 5-7, FIG. 42A). Likewise, non-nicking BE2max outperformed PE2 by 1.4-fold on average at these well-positioned bases (FIG. 42A). However, PE3 outperformed BE4max by 2.7-fold, and PE2 outperformed BE2max by 2.0-fold, for cytosines beyond the center of the base editing window (average editing of 40±17% for PE3 vs. 15±18% for BE4max, and 22±11% for PE2 vs. 11±13% for BE2max). Overall, indel frequencies for PE2 were very low (averaging 0.86±0.47%), and for PE3 were similar to or modestly higher than that of BE4max (BE4max range: 2.5% to 14%; PE3 range: 2.5% to 21%) (FIG. 42B).

When comparing the efficiency of base editing to prime editing for installation of precise C•G-to-T•A edits (without any bystander editing), the efficiency of prime editing greatly exceeded that of base editing at the above sites, which like most genomic DNA sites, contain multiple cytosines within the ˜5-bp base editing window (FIG. 42C). At these sites, such as EMX1, which contains cytosines at protospacer positions C5, C6, and C7, BE4max generated few products containing only the single target base pair conversion with no bystander edits. In contrast, prime editing at this site could be used to selectively install a C•G-to-T•A edit at any position or combination of positions (C5, C6, C7, C5+C6, C6+C7, C5+C7, or C5+C6+C7) (FIG. 42C). All precise one-base or two-base edits (that is, edits that do not modify any other nearby bases) were much more efficient with PE3 or PE2 than with BE4max or BE2, respectively, while the three-base C•G-to-T•A edit was more efficient with BE4max (FIG. 42C), reflecting the propensity of base editors to edit all target bases within the activity window. Taken together, these results demonstrate that cytosine base editors can result in higher levels of editing at optimally positioned target bases than PE2 or PE3, but prime editing can outperform base editing at non-optimally positioned target bases, and can edit with much higher precision with multiple editable bases.

A•T-to-G•C editing was compared at two genomic loci by an optimized non-nicking ABE (ABEmax¹⁵² with a dCas9 instead of a Cas9 nickase, hereafter referred to as ABEdmax) versus PE2, and by the optimized nicking adenine base editor ABEmax versus PE3. At a site that contains two target adenines in the base editing window (HEK3), ABEs were more efficient than PE2 or PE3 for conversion of A5, but PE3 was more efficient for conversion of A8, which lies at the edge of the ABEmax editing window (FIG. 42D). When comparing the efficiency of precision edits in which only a single adenine is converted, PE3 outperformed ABEmax at both A5 and A8 (FIG. 42E). Overall, ABEs produced far fewer indels at HEK3 than prime editors (0.19±0.02% for ABEdmax vs. 1.5±0.46% for PE2, and 0.53±0.16% for ABEmax vs. 11±2.3% for PE3, FIG. 42F). At FANCF, in which only a single A is present within the base editing window, ABE2 and ABEmax outperformed their prime editing counterparts in total target base pair conversion by 1.8- to 2.9-fold, with virtually all edited products from both base editing and prime editing containing only the precise edit (FIGS. 42D-42E). As with the HEK3 site, ABEs produced far fewer indels at the FANCF site (FIG. 42F).

Collectively, these results indicate that base editing and prime editing offer complementary strengths and weaknesses for making targeted transition mutations. For cases in which a single target nucleotide is present within the base editing window, or when bystander edits are acceptable, current base editors are typically more efficient and result in fewer indels than prime editors. When multiple cytosines or adenines are present and bystander edits are undesirable, or when target bases are poorly positioned for base editing relative to available PAMs, prime editors offer substantial advantages.

### Off-Target Prime Editing

To result in productive editing, prime editing requires target locus:pegRNA spacer complementary for the Cas9 domain to bind, target locus:pegRNA PBS complementarity for pegRNA-primed reverse transcription to initiate, and target locus:reverse transcriptase product complementarity for flap resolution. These three distinct DNA hybridization requirements can minimize off-target prime editing compared to that of other genome editing methods. To demonstrate this, HEK293T cells were treated with PE3 or PE2 and 16 total pegRNAs designed to target four on-target genomic loci, with Cas9 and the four corresponding sgRNAs targeting the same protospacers, or with Cas9 and the same 16 pegRNAs. These four target loci were chosen because each has at least four well-characterized off-target sites for which Cas9 and the corresponding on-target sgRNA in HEK293T cells is known to cause substantial off-target DNA modification^{118,159}. Following treatment, the four on-target loci and the top four known Cas9 off-target sites for each on-target spacer, were sequenced, for a total of 16 off-target sites (Table 1).

Consistent with previous studies¹¹⁸, Cas9 and the four target sgRNAs modified all 16 of the previously reported off-target loci (FIG. 42G). Cas9 off-target modification efficiency among the four off-target sites for the HEK3 target locus averaged 16%. Cas9 and the sgRNA targeting HEK4 resulted in an average of 60% modification of the four tested known off-target sites. Likewise, off-target sites for EMX1 and FANCF were modified by Cas9:sgRNA at an average frequency of 48% and 4.3%, respectively (FIG. 42G). It was noted that pegRNAs with Cas9 nuclease modified on-target sites at similar (1- to 1.5-fold lower) efficiency on average compared to sgRNAs, while pegRNAs with Cas9 nuclease modified off-target sites at ˜4-fold lower average efficiency than sgRNAs.

Strikingly, PE3 or PE2 with the same 16 tested pegRNAs containing these four target spacers resulted in much lower off-target editing (FIG. 42H). Of the 16 sites known to undergo off-target editing by Cas9+sgRNA, PE3+pegRNAs or PE2+pegRNAs resulted in detectable off-target prime editing at only 3 of 16 off-target sites, with only 1 of 16 showing off-target editing efficiency ≥1% (FIG. 42H). Average off-target prime editing for the pegRNAs targeting HEK3, HEK4, EMX1, and FANCF at these 16 known Cas9 off-target sites was <0.1%, <2.2±5.2%, <0.1%, and <0.13±0.11%, respectively (FIG. 42H). Notably, at the HEK4 off-target 3 site that Cas9+pegRNA1 edits with 97% efficiency, PE2+pegRNA1 results in only 0.7% off-target editing despite sharing the same spacer sequence, demonstrating how the two additional DNA hybridization events required for prime editing compared to Cas9 editing can greatly reduce off-target editing. Taken together, these results suggest that PE3 and pegRNAs induce much lower off-target DNA editing in human cells than Cas9 and sgRNAs that target the same protospacers.

Reverse transcription of 3′-extended pegRNAs in principle can proceed into the guide RNA scaffold. If the resulting 3′ flap, despite a lack of complementary at its 3′ end with the unedited DNA strand, is incorporated into the target locus, the outcome is insertion of pegRNA scaffold nucleotides that contributes to indel frequency. We analyzed sequencing data from 66 PE3-mediated editing experiments at four loci in HEK293T cells and observed pegRNA scaffold insertion at a low frequency, averaging 1.7±1.5% total insertion of any number of pegRNA scaffold nucleotides (FIGS. 56A-56D). Inaccessibility of the guide RNA scaffold to the reverse transcriptase due to Cas9 domain binding, as well as cellular excision during flap resolution of the mismatched 3′ end of the 3′ flap that results from pegRNA scaffold reverse transcription, can minimize products that incorporate pegRNA scaffold nucleotides. While such events are rare, future efforts to engineer pegRNAs or prime editor proteins that minimize pegRNA scaffold incorporation may further decrease indel frequencies.

Deaminases in some base editors can act in a Cas9-independent manner, resulting in low-level but widespread off-target DNA editing among first-generation CBEs (but not ABEs)¹⁶⁰⁻¹⁶² and off-target RNA editing among first-generation CBEs and ABEs¹⁶³⁻¹⁶⁵, although newer CBE and ABE variants with engineered deaminases greatly reduce Cas9-independent off-target DNA and RNA editing¹⁶³⁻¹⁶⁵. Prime editors lack base-modification enzymes such as deaminases, and therefore have no inherent ability to modify DNA or RNA bases in a Cas9-independent manner.

While the reverse transcriptase domain in prime editors in principle could process properly primed RNA or DNA templates in cells, it was noted that retrotransposons such as those in the LINE-1 family¹⁶⁶, endogenous retroviruses^{167,168}, and human telomerase all provided active endogenous human reverse transcriptases. Their natural presence in human cells suggests that reverse transcriptase activity itself is not substantially toxic. Indeed, no PE3-dependent differences were observed in HEK293T cell viability compared to that of controls expressing dCas9, Cas9 H840A nickase, or PE2 with R110S+K103L (PE2-dRT) mutations that inactivate the reverse transcriptase¹⁶⁹ (FIGS. 49A-49B).

The above data and analyses notwithstanding, additional studies are needed to assess off-target prime editing in an unbiased, genome-wide manner, as well as to characterize the extent to which the reverse transcriptase variants in prime editors, or prime editing intermediates, may affect cells.

### Prime Editing Pathogenic Transversion, Insertion, and Deletion Mutations in Human Cells

The ability of PE3 to directly install or correct in human cells transversion, small insertion, and small deletion mutations that cause genetic diseases, was tested. Sickle cell disease is most commonly caused by an A•T-to-T•A transversion mutation in HBB, resulting in the mutation of Glu6→Val in beta-globin. Treatment of hematopoietic stem cells ex vivo with Cas9 nuclease and a donor DNA template for HDR, followed by enrichment of edited cells, transplantation, and engraftment is a promising potential strategy for the treatment of sickle-cell disease¹⁷⁰. However, this approach still generates many indel-containing byproducts in addition to the correctly edited HBB allele¹⁷⁰⁻¹⁷¹. While base editors generally produce far fewer indels, they cannot currently make the T•A-to-A•T transversion mutation needed to directly restore the normal sequence of HBB.

PE3 was used to install the HBB E6V mutation in HEK293T cells with 44% efficiency and 4.8% indels (FIG. 43A. From the mixture of PE3-treated cells, we isolated six HEK293T cell lines that are homozygous (triploid) for the HBB E6V allele (FIGS. 53A-53D), demonstrating the ability of prime editing to generate human cell lines with pathogenic mutations. To correct the HBB E6V allele to wild-type HBB, we treated homozygous HBB E6V HEK293T cells with PE3 and a pegRNA programmed to directly revert the HBB E6V mutation to wild-type HBB. In total, 14 pegRNA designs were tested. After three days, DNA sequencing revealed that all 14 pegRNAs when combined with PE3 gave efficient correction of HBB E6V to wild-type HBB (≥26% wild-type HBB without indels), and indel levels averaging 2.8±0.70% (FIG. 50A). The best pegRNA resulted in 52% correction of HBB E6V to wild-type with 2.4% indels (FIG. 43A). Introduction of a silent mutation that modifies the PAM recognized by the pegRNA modestly improved editing efficiency and product purity, to 58% correction with 1.4% indels (FIG. 43A). These results establish that prime editing can install and correct a pathogenic transversion point mutation in a human cell line with high efficiency and minimal byproducts.

Tay-Sachs disease is most often caused by a 4-bp insertion into the HEXA gene (HEXA 1278+TATC)¹³⁶. PE3 was used to install this 4-bp insertion into HEK293T cells with 31% efficiency and 0.8% indels (FIG. 43B), and isolated two HEK293T cell lines that are homozygous for the HEXA 1278+TATC allele (FIGS. 53A-53D). These cells were used to test 43 pegRNAs and three nicking sgRNAs with PE3 or PE3b systems for correction of the pathogenic insertion in HEXA (FIG. 50B), either by perfect reversion to the wild-type allele or by a shifted 4-bp deletion that disrupts the PAM and installs a silent mutation. Nineteen of the 43 pegRNAs tested resulted in ≥20% editing. Perfect correction to wild-type HEXA with PE3 or PE3b and the best pegRNA proceeded with similar average efficiencies (30% for PE3 vs. 33% for PE3b), but the PE3b system was accompanied by 5.3-fold fewer indel products (1.7% for PE3 vs. 0.32% for PE3b) (FIG. 43B and FIG. 50B). These findings demonstrate the ability of prime editing to make precise small insertions and deletions that install or correct a pathogenic allele in mammalian cells efficiently and with a minimum of byproducts.

Finally, the installation of a protective SNP into PRNP, the gene encoding the human prion protein (PrP), was tested. PrP misfolding causes progressive and fatal neurodegenerative prion disease that can arise spontaneously, through inherited dominant mutations in the PRNP gene, or through exposure to misfolded PrP¹⁷². A PRNP G127V mutant allele confers resistance to prion disease in humans¹³⁸ and mice¹⁷³. PE3 was used to install G127V into the human PRNP allele in HEK293T cells, which requires a G•C-to-T•A transversion. Four pegRNAs and three nicking sgRNAs were evaluated with the PE3 system. After three days of exposure to the most effective PE3 and pegRNA, DNA sequencing revealed 53±11% efficiency of installing the G127V mutation and indel levels of 1.7±0.7% (FIG. 43C). Taken together, these results establish the ability of prime editing in human cells to install or correct transversion, insertion, or deletion mutations that cause or confer resistance to disease efficiently, and with a minimum of byproducts.

### Prime Editing in Various Human Cell Lines and Primary Mouse Neurons

Next, prime editing was tested for its ability to edit endogenous sites in three additional human cell lines. In K562 (leukemic bone marrow) cells, PE3 was used to perform transversion edits in the HEK3, EMX1, and FANCF sites, as well as the 18-bp insertion of a 6×His tag in HEK3. An average editing efficiency of 15-30% was observed for each of these four PE3-mediated edits, with indels averaging 0.85-2.2% (FIG. 43A). In U2OS (osteosarcoma) cells, transversion mutations in HEK3 and FANCF were installed, as well as a 3-bp insertion and 6×His tag insertion into HEK3, with 7.9-22% editing efficiency that exceeded indel formation 10- to 76 fold (FIG. 43A). Finally, in HeLa (cervical cancer) cells, a 3-bp insertion into HEK3 was performed, with 12% average efficiency and 1.3% indels (FIG. 43A). Collectively, these data indicate that multiple cell lines beyond HEK293T cells support prime editing, although editing efficiencies vary by cell type and are generally less efficient than in HEK293T cells. Editing:indel ratios remained high in all tested human cell lines.

To determine if prime editing is possible in post-mitotic, terminally differentiated primary cells, primary cortical neurons harvested from E18.5 mice were transduced with a dual split-PE3 lentiviral delivery system in which split-intein splicing²⁰³ reconstitutes PE2 protein from N-terminal and C-terminal halves, each delivered from a separate virus. To restrict editing to post-mitotic neurons, the human synapsin promoter, which is highly specific for mature neurons²⁰⁴, was used to drive expression of both PE2 protein components. GFP was fused through a self-cleaving P2A peptide²⁰⁵ to the N-terminal half of PE2. Nuclei from neurons were isolated two weeks following dual viral transduction and were sequenced directly, or sorted for GFP expression before sequencing. A 7.1±1.2% average prime editing to install a transversion at the DNMT1 locus with 0.58±0.14% average indels in sorted nuclei (FIG. 43D was observed. Cas9 nuclease in the same split-intein dual lentivirus system resulted in 31±5.5% indels among sorted cortical neuron nuclei (FIG. 43D. These data indicate that post-mitotic, terminally differentiated primary cells can support prime editing, and thus establish that prime editing does not require cell replication.

Prime Editing Compared with Cas9-Initiated HDR

The performance of PE3 was compared with that of optimized Cas9-initiated HDR^{128,125} in mitotic cell lines that support HDR¹²⁸. HEK293T, HeLa, K562 and U2OS cells were treated with Cas9 nuclease, a sgRNA, and an ssDNA donor oligonucleotide template designed to install a variety of transversion and insertion edits (FIGS. 43E-43G, and FIGS. 51A-51F). Cas9-initiated HDR in all cases successfully installed the desired edit, but with far higher levels of byproducts (predominantly indels), as expected from treatments that cause double-stranded breaks. Using PE3 in HEK293T cells, HBB E6V installation and correction proceeded with 42% and 58% average editing efficiency with 2.6% and 1.4% average indels, respectively (FIG. 43E and FIG. 43G). In contrast, the same edits with Cas9 nuclease and an HDR template resulted in 5.2% and 6.7% average editing efficiency, with 79% and 51% average indel frequency (FIG. 43E and FIG. 43G). Similarly, PE3 installed PRNP G127V with 53% efficiency and 1.7% indels, whereas Cas9-initiated HDR installed this mutation with 6.9% efficiency and 53% indels (FIG. 43E and FIG. 43G). Thus, the ratio of editing:indels for HBB E6V installation, HBB E6V correction, and PRNP G127V installation on average was 270-fold higher for PE3 than for Cas9-initiated HDR.

Comparisons between PE3 and HDR in human cell lines other than HEK293T showed similar results, although with lower PE3 editing efficiencies. For example, in K562 cells, PE3-mediated 3-bp insertion into HEK3 proceeded with 25% efficiency and 2.8% indels, compared with 17% editing and 72% indels for Cas9-initiated HDR, a 40-fold editing:indel ratio advantage favoring PE3 (FIGS. 43F-43G). In U2OS cells, PE3 performed this 3-bp insertion with 22% efficiency and 2.2% indels, while Cas9-initiated HDR resulted in 15% editing with 74% indels, a 49-fold lower editing:indel ratio (FIGS. 43F-43G). In HeLa cells, PE3 made this insertion with 12% efficiency and 1.3% indels, versus 3.0% editing and 69% indels for Cas9-initiated HDR, a 210-fold editing:indel ratio difference (FIGS. 43F-43G). Collectively, these data indicated that HDR typically results in similar or lower editing efficiencies and far higher indels than PE3 in the four cell lines tested (FIGS. 51A-51F).

### Discussion and Future Directions

The ability to insert DNA sequences with single-nucleotide precision is an especially enabling prime editing capability. For example, PE3 was used to precisely insert into the HEK3 locus in HEK293T cells a His6tag (18 bp, 65% average efficiency), a FLAG epitope tag (24 bp, 18% average efficiency), and an extended LoxP site (44 bp, 23% average efficiency) that is the native substrate for Cre recombinase. Average indels ranged between 3.0% and 5.9% for these examples (FIG. 43H). Many biotechnological, synthetic biology, and therapeutic applications are envisioned to arise from the ability to efficiently and precisely introduce new DNA sequences into target sites of interest in living cells.

Collectively, the prime editing experiments described herein installed 18 insertions up to 44 bp, 22 deletions up to 80 bp, 113 point mutations including 77 transversions, and 18 combination edits, across 12 endogenous loci in the human and mouse genomes at locations ranging from 3 bp upstream to 29 bp downstream of the start of a PAM without making explicit double-stranded DNA breaks. These results establish prime editing as a remarkably versatile genome editing method. Because the overwhelming majority (85-99%) of insertions, deletions, indels, and duplications in ClinVar are ≤30 bp (FIGS. 52A-52D), in principle prime editing can correct up to ˜89% of the 75,122 currently known pathogenic human genetic variants in ClinVar (transitions, transversions, insertions, deletions, indels, and duplications in FIG. 38A), with additional potential to ameliorate diseases caused by copy number gain or loss.

Importantly, for any desired edit the flexibility of prime editing offers many possible choices of pegRNA-induced nick locations, sgRNA-induced second nick locations, PBS lengths, RT template lengths, and which strand to edit first, as demonstrated extensively herein. This flexibility, which contrasts with more limited options typically available for other precision genome editing methods^{125,142,154}, allows editing efficiency, product purity, DNA specificity, or other parameters to be optimized to suit the needs of a given application, as shown in FIGS. 50A-50B in which testing 14 and 43 pegRNAs covering a range of prime editing strategies optimized correction of pathogenic HBB and HEXA alleles, respectively.

By enabling highly precise targeted transitions, transversions, small insertions, and small deletions in the genomes of mammalian cells without requiring double-stranded breaks or HDR, however, prime editing provides a new “search-and-replace” capability that substantially expands the scope of genome editing.

### Example 2: pegRNA Modifications

Described herein is a series of pegRNA designs and strategies that can improve prime editing (PE) efficiency.

Prime editing (PE) is a genome editing technology that can replace, insert, or remove defined DNA sequences within a targeted genetic locus using information encoded within a prime editing guide RNA (pegRNA). Prime editors (PEs) consist of a sequence-programmable DNA binding protein with nuclease activity (Cas9) fused to a polymerase, such as a reverse transcriptase (RT) enzyme. PEs form complexes with pegRNAs, which contain the information for targeting specific DNA loci within their spacer sequences, as well as information specifying the desired edit in an engineered extension built into a standard sgRNA scaffold. PE:pegRNA complexes bind and nick the programmed target DNA locus, allowing hybridization of the nicked DNA strand to the engineered primer binding sequence (PBS) of the pegRNA. The reverse transcriptase domain then copies the edit-encoding information within the RT template portion of the pegRNA, using the nicked genomic DNA as a primer for DNA polymerization. Subsequent DNA repair processes incorporate the newly synthesized edited DNA strand into the genomic locus. Improvements to the design of these pegRNAs can result in improved PE efficiency, as well as enable installation of longer inserted sequences into the genome.

Described herein is a series of pegRNA designs that are envisioned to improve the efficacy of PE. These designs take advantage of a number of previously published approaches for improving sgRNA efficacy and/or stability, as well as utilize a number of novel strategies. These improvements can belong to one or more of a number of different categories:
(1) Longer pegRNAs. This category relates to improved designs that enable efficient expression of functional pegRNAs from non-polymerase III (pol III) promoters, which would enable the expression of longer pegRNAs without burdensome sequence requirements;
(2) Core improvements. This category relates to improvements to the core, Cas9-binding pegRNA scaffold, which could improve efficacy;
(3) RT processivity. This category relates to modifications to the pegRNA that improve RT processivity, enabling the insertion of longer sequences at targeted genomic loci; and
(4) Termini motifs. This category relates to the addition of RNA motifs to the 5′ and/or 3′ termini of the pegRNA that improve pegRNA stability, enhance RT processivity, prevent mis-folding of the pegRNA, or recruit additional factors important for genome editing.

Described herein are a number of potential such pegRNA designs in each category. Several of these designs have been previously described for improving sgRNA activity with Cas9 and are indicated as such. Described herein is also a platform for the evolution of pegRNAs for given sequence targets that would enable the polishing of the pegRNA scaffold and enhance PE activity (5). Notably, these designs could also be readily applied to improve pegRNAs recognized by any Cas9 or evolved variant thereof.

### (1) Longer Peg RNAs.

sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing¹⁸³. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs¹⁸³. However, these promoters are typically partially transcribed, which would result in extra sequence 5′ of the spacer in the expressed pegRNA, which has been shown to result in markedly reduced Cas9:sgRNA activity in a site-dependent manner. Additionally, while pol III-transcribed pegRNAs can simply terminate in a run of 6-7 U's, pegRNAs transcribed from pol II or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA-(lncRNA) tagged sgRNAs¹⁸³. These platforms include RNAs expressed from pCMV and that terminate in the ENE element from the MALAT1 ncRNA from humans¹⁸⁴, the PAN ENE element from KSHViss, or the 3′ box from U1 snRNA¹⁸⁶. Notably, the MALAT1 ncRNA and PAN ENEs form triple helices protecting the polyA-tail^{184, 187}. In addition to enabling expression of RNAs, these constructs could also enhance RNA stability (see section iv). Using the promoter from the U1 snRNA to enable expression of these longer sgRNAs¹⁸³ was also explored. These expression systems will also enable the expression of longer pegRNAs. In addition, a series of methods have been designed for the cleavage of the portion of the pol II promoter that would be transcribed as part of the pegRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy4¹⁹³ and also leads to processing of the guide. Also, incorporation of multiple ENE motifs can lead to improved pegRNA expression and stability. Circularizing the pegRNA in the form of a circular intronic RNA (ciRNA) can lead to enhanced RNA expression and stability, as well as nuclear localization¹⁹⁴. Exemplary pegRNA expression platforms are represented by SEQ ID NOs: 241-245.

### (2) Core/Scaffold Improvements.

The core, Cas9-binding pegRNA scaffold can likely be improved to enhance PE activity. In an exemplary approach, the first pairing element of the scaffold (P1) contains a GTTTT-AAAAC (SEQ ID NO: 246) pairing element. Such runs of Ts can result in pol III pausing and premature termination of the RNA transcript. Rational mutation of one of the T-A pairs to a G-C pair in this portion of P1 can enhance sgRNA activity. This approach can be used to improve pegRNAs. Additionally, increasing the length of P1 can enhance sgRNA folding and lead to improved activity. Finally, it is likely the polishing of the pegRNA scaffold through directed evolution of pegRNAs on a given DNA target would also result in improved activity. This is described in section (v). Exemplary modified pegRNAs are represented by SEQ ID NOs: 247 and 248.

A number of structural modifications to the gRNA scaffold were also tested, none of which showed a significant increase in editing activity (see FIG. 82 at 3.30.13 through 3.30.19 in the X axis, as compare to 3.30). However, this data has two caveats worth noting. First, this guide already worked quite well, and a less effective guide would have been better to test. Second, in HEK cells, transfection is quite efficient, and it was noted that the amount of guide RNA transfected is in large excess compared to what is needed (reducing the amount by ˜4-8 fold has no effect on editing). These improvements might only be seen in other cell types, where transfection efficiency is lower, or with less effective guides. Many of these changes are precedented to improve sgRNA activity in other cell lines.

The sequences of the constructs of FIG. 82 are as follows:

HEK3.30 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Templateand PBS-
UCUGCCAUCAAAGCGUGCUCAGUCUG-Terminal motif-UUUU
(SEQ ID NO: 429)
HEK3.30.0 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-UUUUUU
(SEQ ID NO: 430)
HEK3.30.1 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-[none]
(SEQ ID NO: 431)
HEK3.30.2 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGCUCGAGGCGGAAACGCCUCGAGCUUUU
(SEQ ID NO: 432)
HEK3.30.2b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGCUCGAGGCGGAAACGCCUCGAGC
(SEQ ID NO: 433)
HEK3.30.3 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGCUCGAGGCGUACGCGAAAGCGUACGCCUCGAGCUUUU
(SEQ ID NO: 434)
HEK3.30.3b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGCUCGAGGCGUACGCGAAAGCGUACGCCUCGAGC
(SEQ ID NO: 435)
HEK3.30.5 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGCUCGAGGCGUACGCCCGAUGAAAAUCGGGCGUACGCCUCGAGCUUUU
(SEQ ID NO: 436)
HEK3.30.5a pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUUGGGGUUGGGGUUGGGGUUGGGGUUUU
(SEQ ID NO: 437)
HEK3.30.5b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCGUGCUCAGUCUG-Terminal motif-
UUUGGUGGUGGUGGUUUU
(SEQ ID NO: 438)
HEK3.30.13 pegRNA sequence: spacer-GGCCCAGACUGAGCACGUGA-scaffold-
GUUUUAGAGCUAGCGAAAGCUAGCAAGUUAAAAUAAGGCUAGUCCGUUAUC
AACUUGAAAAAGUGGGACCGAGUCGGUCC-Template and PBS-
UCUGCCAUCAAAGCGUGCUCAGUCUG-Terminal motif-UUUU
(SEQ ID NO: 439)
HEK3.30.15 pegRNA sequence: spacer-GGCCCAGACUGAGCACGUGA-scaffold-
GUUUUAGAGCUAGCUCGAAAGAGCUAGCAAGUUAAAAUAAGGCUAGUCCGU
UAUCAACUUGAAAAAGUGGGACCGAGUCGGUCC-Template and PBS-
UCUGCCAUCAAAGCGUGCUCAGUCUG-Terminal motif-UUUU
(SEQ ID NO: 440)
HEK3.30.15 pegRNA sequence: spacer-GGCCCAGACUGAGCACGUGA-scaffold-
GUUUUAGAGCUAGCUCAUGAAAAUGAGCUAGCAAGUUAAAAUAAGGCUAGU
CCGUUAUCAACUUGAAAAAGUGGGACCGAGUCGGUCC-Template and PBS-
UCUGCCAUCAAAGCGUGCUCAGUCUG-Terminal motif-UUUU
(SEQ ID NO: 441)
HEK3.30.16 pegRNA sequence: spacer-GGCCCAGACUGAGCACGUGA-scaffold-
GUUUUAGAGCUAGCUCAUCCGAAAGGAUGAGCUAGCAAGUUAAAAUAAGGC
UAGUCCGUUAUCAACUUGAAAAAGUGGGACCGAGUCGGUCC-Template and
PBS-UCUGCCAUCAAAGCGUGCUCAGUCUG-Terminal motif-UUUU
(SEQ ID NO: 442)
HEK3.30.17 pegRNA sequence: spacer-GGCCCAGACUGAGCACGUGA-scaffold-
GUUUUAGAGCUAGCUCAUCCUGGAAACAGGAUGAGCUAGCAAGUUAAAAUA
AGGCUAGUCCGUUAUCAACUUGAAAAAGUGGGACCGAGUCGGUCC-Template
and PBS-UCUGCCAUCAAAGCGUGCUCAGUCUG-Terminal motif-UUUU
(SEQ ID NO: 443)
HEK3.30.18 pegRNA sequence: spacer-GGCCCAGACUGAGCACGUGA-scaffold-
GUUUGAGAGCUAGAAAUAGCAAGUUUAAAUAAGGCUAGUCCGUUAUCAACU
UGAAAAAGUGGGACCGAGUCGGUCC-Template and PBS-
UCUGCCAUCAAAGCGUGCUCAGUCUG-Terminal motif-UUUU
(SEQ ID NO: 444)
HEK3.30.19 pegRNA sequence: spacer-GGCCCAGACUGAGCACGUGA-scaffold-
GUUUGAGAGCUAGCUCAUGAAAAUGAGCUAGCAAGUUUAAAUAAGGCUAGU
CCGUUAUCAACUUGAAAAAGUGGGACCGAGUCGGUCC-Template and PBS-
UCUGCCAUCAAAGCGUGCUCAGUCUG-Terminal motif-UUUU
(SEQ ID NO: 445)
HEK3.56 pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAAAGCUUCGACCGUGCUCAGUCUG-Terminal motif-UUUU
(SEQ ID NO: 446)
HEK3.56.1a pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGGCGAAAGCCUCGUGCUCAGUCUG-Terminal motif-UUUU
(SEQ
ID NO: 447)
HEK3.56.1b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGACGAAAGCCUCGUGCUCAGUCUG-Terminal motif-UUUU
(SEQ
ID NO: 448)
HEK3.56.1c pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGGCGAAAGCCCGUGCUCAGUCUG-Terminal motif-UUUU
(SEQ ID NO: 449)
HEK3.56.2a pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGAUGCGAAAGCAUCUCGUGCUCAGUCUG-Terminal motif-UUUU
(SEQ ID NO: 450)
HEK3.56.2b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGAUGCGAAAGCACCUCGUGCUCAGUCUG-Terminal motif-UUUU
(SEQ ID NO: 451)
HEK3.56.2c pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGAUGCGAAAGCAUCCGUGCUCAGUCUG-Terminal motif-UUUU
(SEQ ID NO: 452)
HEK3.56.3a pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGACAUGCGAAAGCAUGUCUCGUGCUCAGUCUG-Terminal motif-
UUUU
(SEQ ID NO: 453)
HEK3.56.3b pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGACAUGCGAAAGCAGGCCCGUGCUCAGUCUG-Terminal motif-
UUUU
(SEQ ID NO: 454)
HEK3.56.3c pegRNA sequence: spacer-GGCCCAGACUGAGCACGUGA-scaffold-
GUUUUAGAGCUAGAAAUAGCAAGUUAAAAUAAGGCUAGUCCGUUAUCAACU
UGAAAAAGUGGCACCGAGUCGGUGC-Template and PBS-
UCUGCCAUCAGACAUGCGAAAGCAUGUCUCGUGCUCAGUCUG-Terminal motif-
UUUU
(SEQ ID NO: 453)
HEK3.56.4a pegRNA sequence: spacer-GGCCCAGACUGAGCACGUGA-scaffold-
GUUUUAGAGCUAUACGUAGCAAGUUAAAAUAAGGCUAGUCCGUUAUCAACU
UUACGAAGUGGGACCGAGUCGGUCC-Template and PBS-
UCUGCCAUCAAAGCUUCGACCGUGCUCAGUCUG-Terminal motif-UUUU
(SEQ ID NO: 455)
HEK3.56.4b pegRNA sequence: 5′motif-
GCAGACCUAAGUGGUGACAUAUGGUCUG-spacer-
GGCCCAGACUGAGCACGUGA-scaffold-
GUUUUAGAGCUAUACGUAGCAAGUUAAAAUAAGGCUAGUCCGUUAUCAACU
UUACGAAGUGGGACCGAGUCGGUCC-Template and PBS--Terminal motif-UUUU
(SEQ ID NO: 456)
HEK3.56.4c pegRNA sequence: 5′motif-
GCAGACCUAAGUGGUGACAUAUGGUCUG-spacer-
GGCCCAGACUGAGCACGUGA-scaffold-
GUUUUAGAGCUAUACGUAGCAAGUUAAAAUAAGGCUAGUCCGUUAUCAACU
UUACGAAGUGGGACCGAGUCGGUCC-Template and PBS--Terminal motif-UUUU
(SEQ ID NO: 456)
HEK3.56.4d pegRNA sequence: 5′motif-
GCAGACCUAAGUGGUGACAUAUGGUCUG-spacer-
GGCCCAGACUGAGCACGUGA-scaffold-
GUUUUAGAGCUAUACGUAGCAAGUUAAAAUAAGGCUAGUCCGUUAUCAACU
UUACGAAGUGGGACCGAGUCGGUCC-Template and PBS--Terminal motif-UUUU
(SEQ ID NO: 456)

Note that where either no terminal motif or a terminal motif that does not end in a run of U's exists, transcript was terminated using the following HDV ribozyme:

(SEQ ID NO: 457)
GGCCGGCAUGGUCCCAGCCUCCUCGCUGGCGCCGG
CUGGGCAACAUGCUUCGGCAUGGCGAAUGGGAC

(3) Improvement of RT Processivity Via Modifications to the Template Region of the pegRNA

As the size of the insertion templated by the pegRNA increases, it is more likely to be degraded by endonucleases, undergo spontaneous hydrolysis, or fold into secondary structures unable to be reverse-transcribed by the RT or that disrupt folding of the pegRNA scaffold and subsequent Cas9-RT binding. Accordingly, it is likely that modification to the template of the pegRNA might be necessary to affect large insertions, such as the insertion of whole genes. Some strategies to do so include the incorporation of modified nucleotides within a synthetic or semi-synthetic pegRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the pegRNA to enhance stability and activity. Alternatively or additionally, the template of the pegRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also imagine splitting the template into two, separate pegRNAs. In such a design, a PE would be used to initiate transcription and also recruit a separate template RNA to the targeted site via an RNA-binding protein fused to Cas9 or an RNA recognition element on the pegRNA itself such as the MS2 aptamer. The RT could either directly bind to this separate template RNA, or initiate reverse transcription on the original pegRNA before swapping to the second template. Such an approach could enable long insertions by both preventing mis-folding of the pegRNA upon addition of the long template and also by not requiring dissociation of Cas9 from the genome for long insertions to occur, which could possibly be inhibiting PE-based long insertions.

### (4) Installation of Additional RNA Motifs at the 5′ or 3′ Termini

pegRNA designs could also be improved via the installation of additional motifs at either end of the terminus of the RNA. Several such motifs—such as the PAN ENE from KSHV and the ENE from MALAT1 were discussed earlier in part (i)^{184,185} as possible means to terminate expression of longer pegRNAs from non-pol III promoters. These elements form RNA triple helices that engulf the polyA tail, resulting in their being retained within the nucleus^{184,187}. However, by forming complex structures at the 3′ terminus of the pegRNA that occlude the terminal nucleotide, these structures would also likely help prevent exonuclease-mediated degradation of pegRNAs. Other structural elements inserted at the 3′ terminus could also enhance RNA stability, albeit without enabling termination from non-pol III promoters. Such motifs could include hairpins or RNA quadruplexes that would occlude the 3′ terminus 197, or self-cleaving ribozymes such as HDV that would result in the formation of a 2′-3′-cyclic phosphate at the 3′ terminus and also potentially render the pegRNA less likely to be degraded by exonucleases¹⁹⁸. Inducing the pegRNA to cyclize via incomplete splicing—to form a ciRNA—could also increase pegRNA stability and result in the pegRNA being retained within the nucleus¹⁹⁴.

Additional RNA motifs could also improve RT processivity or enhance pegRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹. Addition of dimerization motifs—such as kissing loops or a GNRA tetraloop/tetraloop receptor pair²⁰⁰—at the 5′ and 3′ termini of the pegRNA could also result in effective circularization of the pegRNA, improving stability. Additionally, it is envisioned that addition of these motifs could enable the physical separation of the pegRNA spacer and primer, prevention occlusion of the spacer which would hinder PE activity. Short 5′ extensions to the pegRNA that form a small toehold hairpin in the spacer region could also compete favorably against the annealing region of the pegRNA binding the spacer. Finally, kissing loops could also be used to recruit other template RNAs to the genomic site and enable swapping of RT activity from one RNA to the other (section iii). Exemplary pegRNA constructs are represented by SEQ ID NOs: 251-255.

(5) Evolution of pegRNAs

It is likely that the pegRNA scaffold can be further improved via directed evolution, in an analogous fashion to how SpCas9 and base editors have been improved²⁰¹. Directed evolution could enhance pegRNA recognition by Cas9 or evolved Cas9 variants. Additionally, it is likely that different pegRNA scaffold sequences would be optimal at different genomic loci, either enhancing PE activity at the site in question, reducing off-target activities, or both. Finally, evolution of pegRNA scaffolds to which other RNA motifs have been added would almost certainly improve the activity of the fused pegRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-pegRNA fusions as well. In addition, while Cas9 currently does not generally tolerate 5′ extension of the sgRNA, directed evolution will likely generate enabling mutations that mitigate this intolerance, allowing additional RNA motifs to be utilized.

As described herein, a number of these approaches have already been described for use with Cas9:sgRNA complexes, but no designs for improving pegRNA activity have been reported. Other strategies for the installation of programmable mutations into the genome include base-editing, homology-directed recombination (HDR), precise microhomology-mediated end-joining (MMEJ), or transposase-mediated editing. However, all of these approaches have significant drawbacks when compared to PEs. Current base editors, while more efficient than existing PEs, can only install certain classes of genomic mutations and can result in additional, undesired nucleotide conversions at the site of interest. HDR is only feasible in a very small minority of cell types and results in comparably high rates of random insertion and deletion mutations (indels). Precise MMEJ can lead to predictable repair of double-strand breaks, but is largely limited to installation of deletions, is very site-dependent, and can also have comparably high rates of undesired indels. Transposase-mediated editing has to date only been shown to function in bacteria. As such improvements to PE represent possibly the best path forward for the therapeutic correction of a wide-swatch of genomic mutations.

### (5) PBS Toeloops

In order to further improve PE activity, the inventors contemplated adding a toeloop sequence at the 3′ end of a pegRNA having a 3′ extension arm. FIG. 71A provides an example of a generic SpCas9 pegRNA having a 3′ extension arm (top molecule). The 3′ extension arm, in turn, comprises an RT template (that includes that the desired edit) and a primer binding site (PBS) at the 3′ end of the molecule. The molecule terminates with a poly(U) sequence comprising three U nucleobases (i.e., 5′-UUU-3′).

By contrast, the bottom portion of FIG. 71A shows the same pegRNA molecule as the top portion of FIG. 71A, but wherein a 9-nucleobase sequence of 5′-GAAANNNNN-3′ has been inserted between the 3′ end of the primer binding site and the 5′ end of the terminal poly(U) sequence. This structure folds back on itself by 180° to form a “toeloop” RNA structure, wherein the sequences of 5′-NNNNN-3′ of the 9-nucleobase insertion anneals with a complementary sequence in the primer binding site, and wherein the 5′-GAAA-3′ portion forms the 180° turn. The features of the toeloop sequence depicted in FIG. 71A is not intended to limit or narrow the scope of possible toeloops that could be used in its place. Further, the sequence of the toeloop will depend upon the complementary sequence of the primer binding site. Essentially though, the toeloop sequence, in various embodiments, may have a first sequence portion that forms a 180°, and a second sequence portion that has a sequence that is complentary to a portion of the primer binding site.

Without being bound by theory, the toeloop sequence is thought to enable pegRNA the use of pegRNAs with increasingly longer primer binding sites than would otherwise be possible. Longer PBS sequences, in turn, are thought to improve PE activity. More in particular, the likely function of the toeloop is to occlude or at least minimize the PBS from interacting with the spacer. Stable hairpin formation between the PBS and the spacer can lead to an inactive pegRNA. Without a toeloop, this interaction may require restricting the length of the PBS. Blocking or minimizing the interaction between the spacer and the PBS using a 3′ end toeloop may lead to an improvement in PE activity.

(6) Expression of pegRNAs from Non-Pol III Promoters

A variety of pegRNA expression systems were tested for their ability to generate pegRNAs, using insertion of a 102 nucleotide sequence from FKBP as a readout.

Transcription of pegRNA can be directed by a typical constitutive promoter, such as U6 promoter. Although the U6 promoter is in most cases effective at directing transcription of pegRNAs, the U6 promoter is not very effective at directing the transcription of longer pegRNAs or U-rich RNAs. U-rich RNA stretches of cause premature termination of transcription. This Example compared editing outcomes of guides expressed from the CMV promoter or U1 promoter with the U6 promoter. These promoters require a different terminator sequence, such as MASC ENE or PAN ENE, as provided below. An increase in editing was observed with the pCMV/MASC-ENE system, however these guides resulted in incomplete insertion of the sequence, while, with the U6 promoter, complete insertion was observed at lower levels of editing. See FIG. 81. The data suggests the likelihood that the alternate expression systems may be useful for long insertions.

The nucleotide sequence of the pCMV/MASC-ENE expression systems as follows (5′-to-3′ direction) (with the name of the motif in bold immediately preceding the region to which it refers):

(SEQ ID NO: 458)
-pCMV promoter-
TAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTA
CATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCA
GTACATCTACGTATTAGTCATCGCTATTACCATGGTGATGCGGTTTTGGCAGTAC
ATCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGTGGGA
GGTCTATATAAGCAGAGCTGGTTTAGTGAACCGTCAGATC-Csy4 loop-
GTTCACTGCCGTATAGGCAG-spacer-GGCCCAGACTGAGCACGTGA-scaffold-
GTTTTAGAGCTAGAAATAGCAAGTTAAAATAAGGCTAGTCCGTTATCAACTTGAA
AAAGTGGGACCGAGTCGGTCC-template-
TGGAGGAAGCAGGGCTTCCTTTCCTCTGCCATCA-insert-
AAATTTCTTTCCATCTTCAAGCATCCCGGTGTAGTGCACCACGCAGGTCTGGCCG
CGCTTGGGGAAGGTGCGCCCGTCTCCTGGGGAGATGGTTTCCACCTGCACTCC-
PBS-CGTGCTCAGTCTG-linker-TTT-MASC ENE-
TAGGGTCATGAAGGTTTTTCTTTTCCTGAGAAAACAACACGTATTGTTTTCTCAGG
TTTTGCTTTTTGGCCTTTTTCTAGCTTAAAAAAAAAAAAAGCAAAAGATGCTGGT
GGTTGGCACTCCTGGTTTCCAGGACGGGGTTCAAATCCCTGCGGCGTCTTTGCTTT
GACT-unrelated plasmid sequence-
TTTTTTTAAGCTTGGGCCGCTCGAGGTAGCAGC-Ubc promoter-
GGCCTCCGCGCCGGGTTTTGGCGCCTCCCGCGGGCGCCCCCCTCCTCACGGCGAG
CGCTGCCACGTCAGACGAAGGGCGCAGGAGCGTTCCTGATCCTTCCGCCCGGAC
GCTCAGGACAGCGGCCCGCTGCTCATAAGACTCGGCCTTAGAACCCCAGTATCA
GCAGAAGGACATTTTAGGACGGGACTTGGGTGACTCTAGGGCACTGGTTTTCTTT
CCAGAGAGCGGAACAGGCGAGGAAAAGTAGTCCCTTCTCGGCGATTCTGCGGAG
GGATCTCCGTGGGGCGGTGAACGCCGATGATTATATAAGGACGCGCCGGGTGTG
GCACAGCTAGTTCCGTCGCAGCCGGGATTTGGGTCGCGGTTCTTGTTTGTGGATC
GCTGTGATCGTCACTTGGTGAGTTGCGGGCTGCTGGGCTGGCCGGGGCTTTCGTG
GCCGCCGGGCCGCTCGGTGGGACGGAAGCGTGTGGAGAGACCGCCAAGGGCTGT
AGTCTGGGTCCGCGAGCAAGGTTGCCCTGAACTGGGGGTTGGGGGGAGCGCACA
AAATGGCGGCTGTTCCCGAGTCTTGAATGGAAGACGCTTGTAAGGCGGGCTGTG
AGGTCGTTGAAACAAGGTGGGGGGCATGGTGGGCGGCAAGAACCCAAGGTCTTG
AGGCCTTCGCTAATGCGGGAAAGCTCTTATTCGGGTGAGATGGGCTGGGGCACC
ATCTGGGGACCCTGACGTGAAGTTTGTCACTGACTGGAGAACTCGGGTTTGTCGT
CTGGTTGCGGGGGCGGCAGTTATGCGGTGCCGTTGGGCAGTGCACCCGTACCTTT
GGGAGCGCGCGCCTCGTCGTGTCGTGACGTCACCCGTTCTGTTGGCTTATAATGC
AGGGTGGGGCCACCTGCCGGTAGGTGTGCGGTAGGCTTTTCTCCGTCGCAGGACG
CAGGGTTCGGGCCTAGGGTAGGCTCTCCTGAATCGACAGGCGCCGGACCTCTGGT
GAGGGGAGGGATAAGTGAGGCGTCAGTTTCTTTGGTCGGTTTTATGTACCTATCT
TCTTAAGTAGCTGAAGCTCCGGTTTTGAACTATGCGCTCGGGGTTGGCGAGTGTG
TTTTGTGAAGTTTTTTAGGCACCTTTTGAAATGTAATCATTTGGGTCAATATGTAA
TTTTCAGTGTTAGACTAGTAAATTGTCCGCTAAATTCTGGCCGTTTTTGGCTTTTT
TGTTAGACAGGATCCCCGGGTACCGGTCGCCACC-Csy4
and
NLS-ATGGGCTCTTTTACTATGGACCACTACCTGGATATTAGACTGAGACCTGACCCTG
AGTTCCCACCCGCCCAGCTGATGAGCGTGCTGTTCGGCAAGCTGCACCAGGCCCT
GGTGGCACAGGGAGGCGACCGGATCGGCGTGAGCTTCCCCGACCTGGATGAGAG
CAGATCCAGGCTGGGAGAGCGCCTGAGGATCCACGCATCCGCCGACGATCTGCG
CGCCCTGCTGGCCCGGCCATGGCTGGAGGGCCTGCGCGACCACCTGCAGTTTGG
AGAGCCAGCAGTGGTGCCACACCCTACCCCATACAGGCAGGTGTCCAGGGTGCA
GGCAAAGTCTAACCCTGAGCGGCTGCGGAGAAGGCTGATGCGCCGGCACGATCT
GTCTGAGGAGGAGGCCAGAAAGAGGATCCCCGACACCGTGGCCAGAACACTGG
ATCTGCCTTTCGTGACCCTGCGGAGCCAGAGCACAGGCCAGCACTTCAGACTGTT
TATCAGGCACGGCCCACTGCAGGTGACAGCCGAGGAAGGAGGATTCACTTGTTA
CGGACTGTCTAAAGGAGGATTCGTGCCCTGGTTCAGCAGCCTGAGGCCTCCTAAG
AAGAAGAGGAAGGTTTAA-SV40 terminator-
TGATCATAATCAAGCCATATCACATCTGTAGAGGTTTACTTGCTTTAAAAAACCT
CCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATG
TATCTTATCATGTCTGGATCTGC.

### Key:

[pCMV promoter]—binds pol II RNA polymerase
[Csy4 loop]—bound by Csy4 protein, results in cleavage 3′ of the loop. Required because part of [CMV promoter] is transcribed, and if this sequence is attached 5′ of the gRNA it will lower/eliminate activity (previously known).
[Spacer sequence] of pegRNA
[pegRNA scaffold]
[DNA synthesis template]
[insertion edit (108 nt from FKBP)]
[primer binding site]
[Linker] (highly variable)—connects PBS and terminator element
[MASC ENE transcription terminator]—transcription of this element results in termination of transcription; a polyA tail is encoded and then sequestered by the ENE element
[Unimportant sequence]
[Ubc promoter]—required for expression of the Csy4 protein
[Csy4 protein and NLS]—required for processing of the 5′ end of the guide. Other strategies could also be used that don't require expression of a large protein (such as ribozyme-mediated cleavage of the spacer), but these would require more individual tuning for different spacer sequences, so we used this.
[SV40 terminator]—for termination of the Csy4 protein.

### (7) Additional RNA Motifs

See FIG. 82 for details on certain motifs, such as an HDV ribozyme 3′ of the pegRNA, or G-quadruplex insertion, P1 extensions, template hairpins, and tetraloop circ'd, that may be introduced into a pegRNA to improve its performance.

In particular, this Example tested the effect of installing a tRNA motif 3′ of the primer binding site. This element was chosen because of multiple potential functions:
(1) the tRNA motif is a very stable RNA motif, and so could potentially reduce pegRNA degradation;
(2) the MMLV RT uses a prolyl-tRNA as a primer when converting the viral genome into DNA during transcription, so it was suspected the same cap could be bound by the RT, improving binding of the pegRNA by PE, RNA stability, and bringing the PBS back in closer proximity to the genomic site, potentially also improving activity.

In these constructs, the P1 of the tRNA (see FIG. 84) was extended. P1 refers to the first stem/base-pairing element of the tRNA (see FIG. 84). This was believed to be necessary to prevent RNAseP-mediated cleavage of the tRNA 5′ of the P1, which would result in its removal from the pegRNA.

In this design a prolyl-tRNA (codon CGG) with an extended P1 and short 3 nt linker between the tRNA and the PBS was used. A variety of tRNA designs were tested and the editing efficiency was tested compared to a pegRNA having no tRNA cap—see the comparative data in FIG. 83 (depicting a PE experiment that targeted editing of the HEK3 gene, specifically targeting the insertion of a 10 nt insertion at position +1 relative to the nick site and using PE3), FIG. 85 (depicting a PE experiment that targeted editing of the FANCF gene, specifically targeting a G-to-T conversion at position +5 relative to the nick site and using PE3 construct) and FIG. 86 (depicting a PE experiment that targeted editing of the HEK3 gene, specifically targeting the insertion of a 71 nt FLAG tag insertion at position +1 relative to the nick site and using PE3 construct). tRNA-modified pegRNAs were tested against a non-modified pegRNA control.

UGG/CGG refers to the codon used, the number refers to the length of the added P1 extension, long indicates an 8 nt linker, no designation a 3 nt linker.

The data suggest that the installation of a tRNA may enable use of shorter PBSs, which would likely result in additional activity improvements. In the case of RNF2, it is possible/likely that the linker used resulted in improved PBS binding to the spacer, and the resulting diminishment in activity.

Some sequences used:

HEK3 +1 FLAG-tag insertion, proly-tRNA
{UGG} P1 ext 5 nt, linker 3 nt
(SEQ ID NO: 459)
GGCCCAGACUGAGCACGUGAGUUUUAGAGCUAGAA
AUAGCAAGUUAAAAUAAGGCUAGUCCGUUAUCAA
CUUGAAAAAGUGGCACCGAGUCGGUGCUGGAGGAA
GCAGGGCUUCCUUUCCUCUGCCAUCACUUAUCGUC
GUCAUCCUUGUAAUCCGUGCUCAGUCUGUCUGGCG
GGGCUCGUUGGUCUAGGGGUAUGAUUCUCGCUUCG
GGUGCGAGAGGUCCCGGGUUCAAAUCCCGGACGAG
CCCCGCCUUUU
FANCF +5 G to T proly-tRNA {CGG}+10
P1 ext 5 nt, linker 3 nt
(SEQ ID NO: 460)
GGAAUCCCUUCUGCAGCACCGUUUUAGAGCUAGAA
AUAGCAAGUUAAAAUAAGGCUAGUCCGUUAUCAA
CUUGAAAAAGUGGCACCGAGUCGGUGCGGAAAAGC
GAUCAAGGUGCUGCAGAAGGGAUCUGGCGGGGCUC
GUUGGUCUAGGGGUAUGAUUCUCGCUUCGGGUGCG
AGAGGUCCCGGGUUCAAAUCCCGGACGAGCCCCGC
CUUUU
HEK3 ++1 10 nt insertion, proly-tRNA
{UGG} P1 ext 5 nt, linker 3 nt
(SEQ ID NO: 461)
GGCCCAGACUGAGCACGUGAGUUUUAGAGCUAGAA
AUAGCAAGUUAAAAUAAGGCUAGUCCGUUAUCAA
CUUGAAAAAGUGGGACCGAGUCGGUCCUCUGCCAU
CAAAGCUUCGACCGUGCUCAGUCUUCUGCUCGAGG
CGGGGCUCGUUGGUCUAGGGGUAUGAUUCUCGCUU
CGGGUGCGAGAGGUCCCGGGUUCAAAUCCCGGACG
AGCCCCGCCUCGAGCUUUU

The sequences reported in the data of FIGS. 85 and 86 are as follows:

FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-partial CGG tRNA linker 8-
UCUCUCUCUGGUCUAGGGGUAUGAUUCUCGCUUCGGGUGCGAGAGGUCCCG
GGUUCAAUUUU
(SEQ ID NO: 462)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 5 linker 3-
UCUGGCGGGGCUCGUUGGUCUAGGGGUAUGAUUCUCGCUUUGGGUGCGAGA
GGUCCCGGGUUCAAAUCCCGGACGAGCCCCGCCUUUU
(SEQ ID NO: 463)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 5 linker 8-
UCUCUCUCGGCGGGGCUCGUUGGUCUAGGGGUAUGAUUCUCGCUUUGGGUG
CGAGAGGUCCCGGGUUCAAAUCCCGGACGAGCCCCGCCUUUU
(SEQ ID NO: 464)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 8 linker 3-
UCUCGAGGCGGGGCUCGUUGGUCUAGGGGUAUGAUUCUCGCUUUGGGUGCG
AGAGGUCCCGGGUUCAAAUCCCGGACGAGCCCCGCCUCGUUUU
(SEQ ID NO: 465)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 8 linker 8-
UCUCUCUCCGAGGCGGGGCUCGUUGGUCUAGGGGUAUGAUUCUCGCUUUGG
GUGCGAGAGGUCCCGGGUUCAAAUCCCGGACGAGCCCCGCCUCGUUUU
(SEQ ID NO: 466)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 11 linker 3-
UCUGCUCGAGGCGGGGCUCGUUGGUCUAGGGGUAUGAUUCUCGCUUUGGGU
GCGAGAGGUCCCGGGUUCAAAUCCCGGACGAGCCCCGCCUCGAGCUUUU
(SEQ ID NO: 467)
FANCF +5 G to T pegRNA sequence:
space, scaffold template and PBSr-
GGAAUCCCUUCUGCAGCACCGUUUUAGAGCUAGAAAUAGCAAGUUAAAAUA
AGGCUAGUCCGUUAUCAACUUGAAAAAGUGGCACCGAGUCGGUGCGGAAAA
GCGAUCAAGGUGCUGCAGAAGGGA-UGG P1 ext 11 linker 8-
UCUCUCUCGCUCGAGGCGGGGCUCGUUGGUCUAGGGGUAUGAUUCUCGCUU
UGGGUGCGAGAGGUCCCGGGUUCAAAUCCCGGACGAGCCCCGCCUCGAGCU
UUU
(SEQ ID NO: 468)
HEK3 +1 10 nt insertion pegRNA sequence:
space, scaffold template and PBSr-
GGCCCAGACTGAGCACGTGAGTTTTAGAGCTAGAAATAGCAAGTTAAAATA
AGGCTAGTCCGTTATCAACTTGAAAAAGTGGCACCGAGTCGGTGCTCTGCC
ATCAAAGCTTCGACCGTGCTCAGTCTG-UGG P1 ext 5 linker 3-
UCUGGCGGGGCUCGUUGGUCUAGGGGUAUGAUUCUCGCUUUGGGUGCGAGA
GGUCCCGGGUUCAAAUCCCGGACGAGCCCCGCCUUUU
(SEQ ID NO: 469)
HEK3 +1 FLAG insertion pegRNA sequence:
space, scaffold template and PBSr-
GGCCCAGACUGAGCACGUGAGUUUUAGAGCUAGAAAUAGCAAGUUAAAAUA
AGGCUAGUCCGUUAUCAACUUGAAAAAGUGGCACCGAGUCGGUGCUGGAGG
AAGCAGGGCUUCCUUUCCUCUGCCAUCACUUAUCGUCGUCAUCCUUGUAAU
CCGUGCUCAGUCUG-partial CGG tRNA linker 8-
UCUCUCUCUGGUCUAGGGGUAUGAUUCUCGCUUCGGGUGCGAGAGGUCCCG
GGUUCAAUUUU
(SEQ ID NO: 470)
HEK3 +1 FLAG insertion pegRNA sequence:
space, scaffold template and PBSr-
GGCCCAGACUGAGCACGUGAGUUUUAGAGCUAGAAAUAGCAAGUUAAAAUA
AGGCUAGUCCGUUAUCAACUUGAAAAAGUGGCACCGAGUCGGUGCUGGAGG
AAGCAGGGCUUCCUUUCCUCUGCCAUCACUUAUCGUCGUCAUCCUUGUAAU
CCGUGCUCAGUCUG-UGG P1 ext 5 linker 3-
UCUGGCGGGGCUCGUUGGUCUAGGGGUAUGAUUCUCGCUUUGGGUGCGAGA
GGUCCCGGGUUCAAAUCCCGGACGAGCCCCGCCUUUU
(SEQ ID NO: 471)
HEK3 +1 FLAG insertion pegRNA sequence:
space, scaffold template and PBSr-
GGCCCAGACUGAGCACGUGAGUUUUAGAGCUAGAAAUAGCAAGUUAAAAUA
AGGCUAGUCCGUUAUCAACUUGAAAAAGUGGCACCGAGUCGGUGCUGGAGG
AAGCAGGGCUUCCUUUCCUCUGCCAUCACUUAUCGUCGUCAUCCUUGUAAU
CCGUGCUCAGUCUG-UGG P1 ext 5 linker 8-
UCUCUCUCGGCGGGGCUCGUUGGUCUAGGGGUAUGAUUCUCGCUUUGGGUG
CGAGAGGUCCCGGGUUCAAAUCCCGGACGAGCCCCGCCUUUU
(SEQ ID NO: 472)
HEK3 +1 FLAG insertion pegRNA sequence:
space, scaffold template and PBSr-
GGCCCAGACUGAGCACGUGAGUUUUAGAGCUAGAAAUAGCAAGUUAAAA
UAAGGCUAGUCCGUUAUCAACUUGAAAAAGUGGCACCGAGUCGGUGCUGG
AGGAAGCAGGGCUUCCUUUCCUCUGCCAUCACUUAUCGUCGUCAUCCUUG
UAAUCCGUGCUCAGUCUG-UGG P1 ext 8 linker 8-
UCUCUCUCCGAGGCGGGGCUCGUUGGUCUAGGGGUAUGAUUCUCGCUUUG
GGUGCGAGAGGUCCCGGGUUCAAAUCCCGGACGAGCCCCGCCUCGUUUU
(SEQ ID NO: 473)
HEK3 +1 FLAG insertion pegRNA sequence:
space, scaffold template and PBSr-
GGCCCAGACUGAGCACGUGAGUUUUAGAGCUAGAAAUAGCAAGUUAAAAUA
AGGCUAGUCCGUUAUCAACUUGAAAAAGUGGCACCGAGUCGGUGCUGGAGG
AAGCAGGGCUUCCUUUCCUCUGCCAUCACUUAUCGUCGUCAUCCUUGUAAU
CCGUGCUCAGUCUG-UGG P1 ext 11 linker 8-
UCUCUCUCGCUCGAGGCGGGGCUCGUUGGUCUAGGGGUAUGAUUCUCGCUU
UGGGUGCGAGAGGUCCCGGGUUCAAAUCCCGGACGAGCCCCGCCUCGAGCU
UUU
(SEQ ID NO: 474)
HEK3 +1 FLAG insertion pegRNA sequence:
space, scaffold template and PBSr-
GGCCCAGACUGAGCACGUGAGUUUUAGAGCUAGAAAUAGCAAGUUAAAAUA
AGGCUAGUCCGUUAUCAACUUGAAAAAGUGGCACCGAGUCGGUGCUGGAGG
AAGCAGGGCUUCCUUUCCUCUGCCAUCACUUAUCGUCGUCAUCCUUGUAAU
CCGUGCUCAGUCUG-UGG P1 ext 14 linker 8-
UCUCUCUCGGUGCUCGAGGCGGGGCUCGUUGGUCUAGGGGUAUGAUUCUCG
CUUUGGGUGCGAGAGGUCCCGGGUUCAAAUCCCGGACGAGCCCCGCCUCGA
GCACCUUUU
(SEQ ID NO: 475)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{CGG}-5-
UCUGGCGGGGCUCGUUGGUCUAGGGGUAUGAUUCUCGCUUCGGGUGCGAGA
GGUCCCGGGUUCAAAUCCCGGACGAGCCCCGCC
(SEQ ID NO: 476)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{CGG}-8-
UCUCGAGGCGGGGCUCGUUGGUCUAGGGGUAUGAUUCUCGCUUCGGGUGCG
AGAGGUCCCGGGUUCAAAUCCCGGACGAGCCCCGCCUCGUUUU
(SEQ ID NO: 477)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{CGG}-11-
UCUGCUCGAGGCGGGGCUCGUUGGUCUAGGGGUAUGAUUCUCGCUUCGGGU
GCGAGAGGUCCCGGGUUCAAAUCCCGGACGAGCCCCGCCUCGAGCUUUU
(SEQ ID NO: 478)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{Lys}-5-
UCUGGCGGGCCCGGAUAGCUCAGUCGGUAGAGCAUCAGACUUUUAAUCUGA
GGGUCCAGGGUUCAAGUCCCUGUUCGGGCCCGCCUUUU
(SEQ ID NO: 479)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{Lys}-8-
UCUCGAGGCGGGCCCGGAUAGCUCAGUCGGUAGAGCAUCAGACUUUUAAUC
UGAGGGUCCAGGGUUCAAGUCCCUGUUCGGGCCCGCCUCGUUUU
(SEQ ID NO: 480)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{UGG}-8-
UCUCGAGGCGGGGCUCGUUGGUCUAGGGGUAUGAUUCUCGCUUUGGGUGCG
AGAGGUCCCGGGUUCAAAUCCCGGACGAGCCCCGCCUCGUUUU
(SEQ ID NO: 481)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{UGG}-11-
UCUGCUCGAGGCGGGGCUCGUUGGUCUAGGGGUAUGAUUCUCGCUUUGGGU
GCGAGAGGUCCCGGGUUCAAAUCCCGGACGAGCCCCGCCUCGAGCUUUU
(SEQ ID NO: 482)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{UGG}-8-longer linker-
UCUCUCUCCGAGGCGGGGCUCGUUGGUCUAGGGGUAUGAUUCUCGCUUUGG
GUGCGAGAGGUCCCGGGUUCAAAUCCCGGACGAGCCCCGCCUCGUUUU
(SEQ ID NO: 483)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{UGG}-11-longer linker-
UCUCUCUCGCUCGAGGCGGGGCUCGUUGGUCUAGGGGUAUGAUUCUCGCUU
UGGGUGCGAGAGGUCCCGGGUUCAAAUCCCGGACGAGCCCCGCCUCGAGCUU
UU
(SEQ ID NO: 484)
RNF2 +1 C to A pegRNA sequence:
space, scaffold template and PBSr-
GUCAUCUUAGUCAUUACCUGGUUUUAGAGCUAGAAAUAGCAAGUUAAAAUA
AGGCUAGUCCGUUAUCAACUUGAAAAAGUGGCACCGAGUCGGUGCGAACAC
CUCAUGUAAUGACUAAGAUG-tRNA-Pro{UGG}-14-longer linker-
UCUCUCUCGGUGCUCGAGGCGGGGCUCGUUGGUCUAGGGGUAUGAUUCUCG
CUUUGGGUGCGAGAGGUCCCGGGUUCAAAUCCCGGACGAGCCCCGCCUCGA
GCACCUUUU
(SEQ ID NO: 485)

### Example 3: Next-Generation PEGRNA Modifications for Improving Prime Editing Efficiency

### Background

The prime editor complex consists of two components. The first, the prime editor (PE) itself, in one embodiment, is a programmable nuclease, such as Streptococcus pyogenes Cas9 (SpCas9), fused to a polymerase, such as a reverse transcriptase, and which bears a mutation that inactivates the HNH nuclease domain. The second component is a pegRNA that both targets the editor to a programmed genomic site and contains the template used by the reverse transcriptase to install the programmed edit. Despite its power to create virtually any programmable edit, PE generally has lower activity than base editors (BE) for comparable edits. It is considered that rational engineering of the pegRNA could also lead to improved editing outcomes and enable a wider variety of prime edits in the genomes of cells, e.g., in non-HEK293T cell lines.

It is considered that pegRNAs suffer from both reduced Cas9 affinity and reduced stability relative to canonical single guide RNAs (sgRNAs). This reduction in Cas9 affinity is likely due not only to a 3′ extension, but also due to formation of an RNA duplex between the spacer and primer binding site (PBS) that would inhibit Cas9 binding. Indeed, longer PBS lengths completely obviate PE activity at all sites tested, presumably through this mechanism. Additionally, transfection of pegRNA with SpCas9 nuclease results in fewer indels than sgRNAs targeting the same site, further suggesting that the 3′ extension reduces Cas9 binding and, potentially, catalytic activity. It also seemed likely that the 3′ extension, not being bound and protected by Cas9, could potentially be degraded by exonucleases or be bound by other cellular factors that could compete with Cas9 or RT binding, or otherwise inhibit prime editing. Indeed, upon examining the cellular lifespan of pegRNAs via RT-qPCR, it was observed a significant decrease in stability for the 3′ extension relative to the scaffold region (FIG. 90A-C).

Existing sgRNA Improvement Strategies

Numerous modifications to sgRNAs that improve editing in human cells have been reported. Among the most common such modifications are the ‘flip’ and ‘extension’ mutations to the sgRNA scaffold. It has previously been noted that a four uridine (U) nucleotide stretch in the direct repeat (DR) of the scaffold is a possible polymerase III (pol III) termination sequence, and that flipping of the terminal uridine•adenosine (A) basepair for an A•U basepair results in increased expression and activity of sgRNAs. Similarly, extension of the DR has also been shown to improve sgRNA activity, presumably via stabilization of the Cas9-binding competent structure of the sgRNA. It has been found that such modifications can increase pegRNA activity, just as they have for sgRNA activity (FIG. 91A-D). This is likely due to efficient transfection of the pegRNA-encoding plasmid in HEK293T cells, and it would be expected that these modifications broadly improve activity in other cell types. Another modification sought was to reduce interaction between the spacer and PBS via incorporation of toehold stems.

Next-Generation sgRNA Improvement Strategies

Given the above findings, it was decided to focus on strategies to improve the stability of the 3′ extension. Degradation of the PBS is especially deleterious for pegRNAs because any degradation renders the pegRNA unable to be bound by the RT but could still enable binding by Cas9. Thus, degraded pegRNAs can compete both for Cas9 and for binding to the targeted site, as well as still enable nicking at the site, potentially reducing editing and increasing indel formation.

Thus, it was discovered that the incorporation of structural motifs 3′ of the PBS could lead to improved stability as has been reported for RNA G-quadruplexes appended to sgRNAs. However, it was also decided to screen additional structural motifs since purine-rich sequences could potentially lead to misfolding of the pegRNA. Accordingly, several other structural motifs appended to the PBS by a short, unstructured nucleotide linker were screened.

First, a prequeosine₁-1 riboswitch aptamer—one of the smallest natural tertiary RNA structures—that had been evolved to be more stable, hereafter termed evopreQ₁-1. Second, two structural motifs that could possibly interact with the MMLV RT and thereby result in both improved stability and affinity to PE were selected, namely, the pseudoknot from the MMLV viral genome (here referred to as Mpknot-1) and a modified tRNA that is used by the MMLV RT as a primer for reverse transcription.

The test assay involved screening pegRNA guides configured to encode a FLAG tag insertion sequence—a challenging edit—to be installed at a variety of genomic loci (FIG. 92A-C). Intriguingly, a significant increase in editing activity was observed for a short (G2) quadruplex, as well as both evopreQ1-1 and Mpknot-1 at all sites tested in HEK293T cells, suggesting that these motifs improve activity at a variety of genomic loci.

It was considered that appending structural motifs to the 3′ end of the pegRNA might only improve activity for pegRNAs with longer extensions. To determine if this was the case, a small library of pegRNAs that encode either point mutations or deletions and contain templates of increasing length at 6 additional genomic sites was screened. Broadly improved editing was observed for virtually all guides tested (FIG. 93A-H), with improvement ranging from 1.5-6 fold, irrespective of site, edit type or template length, suggesting their general utility. Interestingly, although incorporation of structural motifs resulted in improved editing versus addition of linker alone, addition of a linker often resulted in improved editing activity relative to the parent pegRNA (FIG. 94). To determine if these structure-tagged pegRNAs result in improved editing in other cell lines, the ability of modified pegRNAs to install a FLAG tag at the HEK3 locus in K562, U20S, and HeLa cells was tested. Large improvements in editing efficacy was observed in these cells when appending either an evopreQ1-1 or Mpknot-1 pseudoknot 3′ of the PBS (FIG. 95A-B).

In order to improve the initial design, it was sought to understand how these motifs were improving activity. Although it seemed likely that they would function via improved cellular lifespan, it was observed that addition of a short, unstructured linker was sometimes sufficient to improve pegRNA activity relative to the parent pegRNA (FIG. 94). Simultaneously, mutations predicted to disrupt the motif structure resulted in reduced editing (FIG. 96) evoPreQ1(mut1) and evoPreQ1(mut2), suggesting that the structure of the motif is important for activity. This in turn suggested that there might be multiple possible mechanisms by which these motifs were increasing PE efficiency.

As a first step, it was sought to confirm that the modified pegRNAs improve the cellular lifetime of pegRNAs. So far, RT-qPCR was used to measure the relative amount of pegRNA scaffold and template, and it was found that appending structured motifs to the 3′ tail of the pegRNA lead to significantly improved amounts of template (FIG. 97). It is considered that the PBS length of these pegRNAs might be able to be increased, further improving editing activity.

It is considered that further improvements to the design of these next generation pegRNAs can be made. To do so, a number of additional 3′ motifs will be screened. These include additional evolved preq1—aptamers, modifications to Mpknot-1, additional natural G-quadruplexes with improved stability, the P4-P6 domain of the group I intron, and the self-cleaving HDV ribozyme. This ribozyme results in RNA processing immediately 5′ of itself, leaving a 2′-3′-cyclicphosphate at the 3′ terminus of the RNA that is resistant to exonucleases. In addition, mutations to the canonical sgRNA scaffold will be tested that have been reported to increase editing efficacy for Cas9 nuclease cutting to see if they improve activity in HEK cells and in other cell types.

These studies have involved a the linker length of 8 nucleotides (nt), however, other linker lengths are possible, including for example, 4, 5, 6, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more nucleotides can be used. In some cases, linker length and sequence will have to be empirically determined for each site. In other cases, a single linker may be used that is pegRNA sequence-agnostic. To aid in this process, a computational script may be used for designing linker sequences that do not interfere with pegRNA structure.

### Additional Designs

As a final step, additional designs of pegRNA were sought. Several aspects of pegRNA structure were considered, including: the pol III promoter used to express pegRNAs, the pegRNA scaffold, and the nicking guide used in PE3 to enhance editing efficacy by nicking the opposing strand. A variety of pol III promoters have been used to express small RNAs in human cells. Historically, two—u6 and h1—have been used for the expression of pre-microRNAs. Of these two, u6 was found to be superior for sgRNA expression. However, other promoters can lead to improved expression of pegRNAs. To determine if this were the case, a number of pol III promoters were screened, including hi and other homologs of u6 for editing efficacy in HEK293T cells. Several promoters, including one homolog of u6 termed u6-9, were found to drastically improve editing efficacy (see FIG. 100A-100E). These promoters are identified as:

Non-limiting examples of U6 promoters include those represented by SEQ ID NOs: 237-240.

### Conclusions

Apart from generally improving PE activity, the modified pegRNAs could simplify the process of designing pegRNAs. Currently, the design of optimal pegRNAs can often require screening 10s-100s of pegRNA constructs. Such testing is especially time-consuming, expensive, and not feasible when constructing a library of pegRNAs. One potential application of such libraries is the systematic tagging of all proteins in a given set. A significant benefit of the modified pegRNAs described herein is that they simplify pegRNA design by limiting the negative effect of poor template choices, as seen for editing at HEK3 (FIG. 93B; FIG. 93E). Additionally, if 3′ motifs enable lengthening the PBS to its maximum possible length (17), this should greatly simplify pegRNA design.

In conclusion, the design of modified pegRNAs with improved editing activity having been validated. These pegRNAs contain a structured RNA 3′ of the PBS and their improved activity is derived from improved cellular lifespan and Cas9 binding activity. These modifications broadly improve PE activity at a wide variety of genomic loci, encoded edits, and cell types.

### Example 4: Engineered pegRNAS that Improve Prime Editing Efficiency

The ability to make targeted changes to the genome of living systems continues to advance the life sciences and medicine. Double-strand break (DSB)-mediated DNA editing strategies that use programmable nucleases such as ZFNs, TALENs, or CRISPR-Cas nucleases can efficiently disrupt genes by inducing insertions or deletions (indels) at the target site, but DSBs also result in outcomes that are often undesired, including uncontrolled mixtures of editing outcomes^{1,2}, larger DNA rearrangements³⁻⁵, p53 activation⁶⁻⁸, and chromothrypsis^{9,10}. Although targeted DSBs can stimulate precise gene correction through homology-directed repair, the process is inefficient in most therapeutically relevant cell types¹¹. In contrast, base editors^{12,13} and prime editors¹⁴ can of efficiently install precise changes in therapeutically relevant cells without requiring DSBs. Cytosine and adenosine base editors enable the conversion of C•G to T•A, and A•T to G•C, respectively, while prime editors enable the installation of virtually any local mutation, including the substitution, insertion, and/or deletion of up to dozens of base pairs at targeted DNA sites.

Prime editing (PE) systems minimally consist of two components: a protein containing a programmable DNA nickase fused to an engineered reverse transcriptase (RT), and a prime editing guide RNA, or pegRNA (FIG. 104A)¹⁴. The pegRNA contains a spacer that specifies the target site, an sgRNA scaffold, and a 3′ extension that encodes the desired edit. This extension contains a primer-binding site (PBS) that is complementary to a portion of the DNA protospacer, and an RT template that encodes the desired edit and downstream genomic sequence. After the PE ribonucleoprotein (RNP) binds the target site and nicks the PAM-containing DNA strand, the resulting nicked DNA strand base pairs to the PBS in the pegRNA, priming the reverse transcription of the RT template directly into the target DNA site¹⁴. The newly synthesized 3′ flap of edited DNA is then resolved by cellular DNA repair pathways, leading to installation of the desired edit at the target site.

The versatility of prime editing arises from the ability of the 3′ extension of the pegRNA to encode a wide variety of edited sequences. Despite its versatility, the efficiency of current prime editors varies substantially among target sites and cell types¹⁴. In this example, it is described that putative degradation of the 3′ extension of pegRNAs can erode prime editing efficiency. Although the resulting truncated pegRNAs compete for target site engagement, they are incompetent for prime editing. To address this vulnerability, RNA motifs that protect pegRNA integrity and broadly improve prime editing efficiencies were identified at a variety of target sites in multiple cell lines and via multiple delivery modalities. The resulting engineered pegRNAs (epegRNAs) substantially advanced the effectiveness, and the application scope, of prime editing.

### Results

RNA Stability Limits pegRNA Efficacy

Unprotected nuclear RNAs are susceptible to degradation from both the 5′ and 3′ termini by exonucleasesis. In contrast to sgRNAs in which the entire guide RNA is protected by an associated Cas9 protein¹⁶, the 3′ extension of pegRNAs is likely to be exposed in cells and thus more susceptible to exonucleolytic degradation. While partially degraded pegRNAs can retain their ability to bind Cas9 and engage the target DNA site, loss or truncation of the PBS might prevent their ability to install the desired edit, thereby occupying PE proteins and target sites with guide RNAs that cannot mediate prime editing.

To demonstrate this possibility, HEK293T cells were transfected with mixtures of two plasmids in varying ratios that generate either a full-length pegRNA containing an RT template encoding a T•A-to-A•T transversion, or a truncated pegRNA containing an RT template encoding a T•A-to-G•C transversion but lacking the PBS at the 3′ terminus. The two pegRNAs targeted either the same or different genomic loci in human cells. The effect of adding a plasmid that generated a non-interacting SaCas9 pegRNA that should compete for transcription with the SpCas9 pegRNA-encoding plasmids, but not interact with the prime editor protein, was also tested. Increasing the production of truncated pegRNA inhibited PE activity when the full-length and truncated pegRNAs were targeted to the same site (FIG. 104B). In contrast, neither a truncated pegRNA targeted to a different genomic site nor a non-targeting SpCas9 sgRNA impeded PE activity any more than the SaCas9 pegRNA (FIG. 104B). These data suggest that degraded pegRNAs with truncated 3′ extensions inhibit PE activity by enabling editing-incompetent prime editor ribonucleoproteins (RNPs) to compete for the targeted genomic locus.

Design of Engineered pegRNAs (epegRNAs) that Improve Prime Editing Efficiency

Having identified truncated pegRNAs as a potent inhibitor of prime editing, it was next sought to minimize pegRNA degradation. It was envisioned that structured RNA motifs at the 3′ end of the pegRNA might improve pegRNA stability, consistent with the ability of RNA structures at the 5′ or 3′ termini to enhance mRNA stability in human cells and in yeast^{17,18}. For instance, the long-noncoding RNA MALAT1 is stabilized by a triple helix that sequesters its poly(A) tail, limiting both degradation and nuclear export¹⁹.

Whether prime editing efficiency could be improved by incorporating additional RNAstructures was tested using one of two stable pseudoknots at the 3′ end of the pegRNA: either a modified prequeosine1-1 riboswitch aptamer20,21, (evopreQ1), or the frameshifting pseudoknot from Moloney murine leukemia virus (MMLV)22, hereafter referred to as “mpknot” (FIG. 108). EvopreQ1 was chosen because it is one of the smallest naturally derived RNA structural motifs with a defined tertiary structure (42 nucleotides, nt, in length)^{20,21}. It was reasoned that smaller motifs would minimize the formation of secondary structures that could interfere with pegRNA function. Furthermore, shorter pegRNAs can be more easily produced by chemical synthesis. Mpknot was chosen because of its tertiary structure and because it is an endogenous a template for the MMLV RT from which the RT in canonical prime editors was engineered, raising the possibility that mpknot might help recruit the RT.

It was tested if these epegRNAs could insert a FLAG epitope tag sequence using PE3 at five genomic loci in HEK293T cells (FIG. 105A). To reduce the potential for the motif to interfere with pegRNA function during prime editing, an 8-nt linker was included to connect either evopreQ₁ or mpknot to the 3′ end of the epegRNA PBS. Linker sequences were designed using ViennaRNA²³ to avoid potential base pairing interactions between the linker and PBS, or between the linker and the pegRNA spacer¹⁴. An average of 2.1-fold increased efficiency of FLAG tag insertion was observed when using epegRNAs compared to canonical pegRNAs across all five genomic sites tested, with no apparent change in edit:indel ratios (FIGS. 109A-109C), suggesting that 3′ terminal pseudoknot motifs can improve PE efficacy.

The role of the linker sequence in editing efficiency was characterized by comparing the ability of epegRNAs with or without 8-nt linkers to mediate transversions or FLAG tag insertions. A decrease in PE3 editing efficiency was observed upon removing the linker for epegRNAs containing the mpknot (p=0.022), but no significant difference for epegRNAs that contain evopreQ₁ was observed (FIG. 110), perhaps because evopreQ1 is smaller than mpknot and is less prone to steric clashes with the RT. While the overall average editing efficiencies for epegRNAs with evopreQ₁ were similar (with or without a linker) occasional reduced performance for epegRNAs without a linker were noted (FIG. 110). Therefore, an 8-nt linker was used unless otherwise noted for all subsequent epegRNA designs.

To ensure that this improvement in PE efficacy was not limited to epegRNAs with longer extensions, 148 additional epegRNAs were tested that encoded a variety of point mutations or deletions with various RT template lengths at seven different genomic sites in HEK293T cells using PE3. Use of either motif resulted in a 1.5-fold average improvement in prime editing efficiency relative to that of canonical pegRNAs across all tested sites and pegRNAs in HEK293T cells, with no apparent change in edit:indel ratios (FIGS. 105B-105C, FIGS. 111A-111K, and FIGS. 112A-112C). Together, these results establish that epegRNAs broadly improve PE efficacy in HEK293T cells.

Engineered pegRNAs Improve Prime Editing in Multiple Mammalian Cell Lines

It was previously observed that PE efficiency varies substantially between mammalian cell types¹⁴, highlighting the need to test improved PE systems in a variety of cells. The ability of epegRNAs containing a 3′ evopreQ₁ or mpknot motif to insert a 24-bp FLAG epitope tag at HEK3, delete 15 bp at DNMT1, or install a C•G-to-A•T transversion at RNF2 via PE3 in K562, U2OS, and HeLa cells were tested. In each of these cell lines, epegRNAs resulted in large improvements in editing efficiency compared to pegRNAs, averaging 2.4-fold higher editing in K562 cells, 3.1-fold higher editing in HeLa cells, and 5.6-fold higher editing in U2OS cells across all tested edits (FIG. 105D) with no decrease in edit:indel ratios (FIGS. 109A-109C). These results indicate that epegRNAs can be used to enhance prime editing in multiple mammalian cell lines. Additionally, epegRNAs improved editing efficiencies to a greater degree in non-HEK293T cells than in HEK293T cells, (FIG. 105A and FIG. 111A-111K compared to FIG. 105D), suggesting that epegRNAs are especially beneficial in cell lines that are less efficiently transfected or edited by the original PE systems.

Effect of Engineered pegRNAs on Off-Target Prime Editing

It was previously demonstrated that prime editing results in substantially less off-target editing than other CRISPR gene editing strategies^{14,24-27}. To determine if the addition of evopreQ₁ or mpknot changed the extent of off-target editing, HEK293T cells were treated with pegRNAs or epegRNAs targeting HEK3, EMX1, or FANCF that template either a transversion (T•A-to-A•T at HEK3 or G•C-to-T•A at EMX1 and FANCF) or a 15-bp deletion using PE3. The extent of indel generation was measured, as well as any nucleotide changes that could reasonably arise from prime editing at the top four experimentally confirmed off-target sites²⁸, for each targeted locus, and the extent of off-target editing between epegRNAs and unmodified pegRNAs was compared following treatment with PE3. In all cases epegRNAs and pegRNAs both exhibited ≤0.1% off-target prime editing and or indels at the examined sites (FIG. 113), suggesting that epegRNAs and pegRNAs exhibit similar levels of off-target editing.

Basis of Enhanced Prime Editing with Engineered pegRNAs

EpegRNAs may enhance prime editing outcomes through a variety of mechanisms, including resistance to degradation, higher expression levels, more efficient Cas9 binding, and/or target DNA engagement when complexed with Cas9. Each of these possibilities were probed.

To determine whether evopreQ₁ or mpknot impede degradation of the pegRNA 3′ extension, the stability of epegRNAs and pegRNAs were compared following in vitro incubation with HEK293T nuclear lysates containing endogenous exonucleases. It was found that pegRNAs were degraded to a greater extent from this treatment compared to epegRNAs (1.9-fold compared to evopreQ₁ and 1.8-fold compared to mpknot, p<0.005, FIG. 106A). Conversely, addition of Cas9, which binds the guide RNA scaffold and is likely to protect the core sgRNA from degradation, rescued pegRNA abundance compared to either epegRNA as determined by RT-qPCR quantification of the guide RNA scaffold (FIG. 106B).

The ability of 3′ structural motifs to increase the abundance of the upstream scaffold region (FIG. 106B) suggests that pegRNA degradation in the nucleus is dominated by 3′-directed degradation. This model is consistent with the characterized behavior of the nuclear exosome, the major source of RNA turnover in the nucleus²⁹. However, partially degraded pegRNAs would generate editing-incompetent RNPs previously shown to inhibit prime editing (FIG. 104C). To detect partially degraded RNAs in cells, lysates of HEK293T cells transfected with plasmids encoding PE2 and either pegRNAs or epegRNAs templating either a +1 FLAG tag insertion at HEK3 or a nucleotide transversion at EMX1 were analyzed via northern blot. RNA species were observed containing the sgRNA scaffold and equivalent in size to the sgRNA, consistent with previous finding (FIG. 106B) that Cas9 binding protects the scaffold from 3′-directed degradation (FIGS. 114A-114C). However, lysates with different total levels of pegRNA or epegRNA had similar levels of sgRNA-like truncated species, which represented only a minority of the guide RNA content of the lysate (FIGS. 114A-114C). Since robust degradation of pegRNAs exposed to nuclear lysate was observed in vitro (FIGS. 106A-106B), and pegRNA is present in levels greater than PE2 in HEK293T cells (FIG. 104B), partially degraded pegRNA species likely do not accumulate at levels amenable to northern blot detection.

Next, genomic prime editing intermediates were examined to better understand how epegRNAs might be mediating improved editing efficiency. In the current model, the 3′ flap intermediate generated by RT extension of the nicked targeted site is converted into a 5′ flap intermediate, replacing the original genomic sequence with the newly synthesized one¹⁴. This 5′ flap is then removed by 5′-3′ exonucleases and the resulting genomic nick undergoes ligation to install the prime edit¹⁴. While full-length pegRNAs would be expected to efficiently template RT extension of the nicked genomic strand, truncated pegRNAs without a PBS should be unable to do so, resulting instead in nicking of the targeted strand followed by chew-back or extension of the strand by DNA repair enzymes (lacking the templated edit in either case). If a greater fraction of RT-extended prime editing intermediates is observed with epegRNAs than with pegRNAs, this would suggest that addition of 3′ RNA motifs improve the integrity of the PBS.

To capture these intermediates, HEK293T cells were transfected with plasmids encoding PE2 and either unmodified pegRNAs or epegRNAs containing evopreQ1 or mpknot that template transversions at HEK3, DNMT1, EMX1, or RNF2. Next, terminal transferase was used to label with oligo-dG the 3′ termini of genomic DNA, which should include intermediates of prime editing that have not yet undergone ligation. In each case, epegRNAs reduced the extent of editing-incompetent intermediates at the targeted site by an average of 2.2-fold across the four sites (FIGS. 106C and 115A-115C). The dominant reverse transcription product contained the full sequence templated by the 3′ extension and two nucleotides templated by the last two nucleotides of the pegRNA scaffold, consistent with previous in vitro characterization of PE intermediates¹⁴. The scaffold-templated nucleotides are presumably removed during DNA repair of the targeted locus to produce the cleanly edited alleles that represent the dominant product of PE. These data are consistent with a model in which epegRNAs improve reverse transcription of the pegRNA extension into the target site by reducing the frequency of unproductive target-site nicking from prime editors bound to truncated pegRNAs.

Because single-stranded 3′ termini are a common feature of 3′ exonuclease substrates³⁰, whether the degradation resistance conferred by these motifs could be explained by the more mechanically stable tertiary structures of pseudoknots was tested next. Notably, appending 15-bp (34-nt) hairpins to the 3′ terminus resulted in inconsistent improvements to PE efficiency compared to appending pseudoknots (FIGS. 116A-116D), suggesting that tertiary structure is indeed an important feature of epegRNAs.

To test if tertiary pseudoknot structure is required for epegRNA-mediated improvements in PE efficiency, editing efficiency of epegRNAs containing the G15C point mutation within evopreQ₁, a mutation known to disrupt pseudoknot formation, was examined (M1 in FIG. 108)²³. epegRNAs were used to install either a 24-bp FLAG epitope tag insertion, a 15-bp deletion, or transversions at HEK3 or RNF2 in HEK293T cells using PE3. Indeed, incorporation of the G15C mutation into evopreQ₁ abolished the increases in editing efficiency (FIG. 106D). These results establish that the secondary or tertiary structure of the motifs are critical for epegRNA-mediated PE improvements, likely by stabilizing the 3′ extension.

Next, the structured 3′ motifs in epegRNAs was tested to determine whether they might increase their expression level compared to pegRNAs. RT-qPCR quantification of the pegRNA scaffold revealed target-dependent differences in epegRNA expression levels relative to unmodified pegRNAs (FIGS. 114A-114C). For a pegRNA that templates a +1 FLAG tag insertion at HEK3, it was observed that addition of evopreQ1 or mpknot decreased pegRNA expression 9.2- to 9.6-fold, despite yielding a 1.9-fold improvement in the efficiency of FLAG tag epitope insertion at HEK3 (FIG. 105A). Similarly, epegRNAs that template a transversion at DNMT1 also exhibited reduced expression (1.6- to 2.1-fold). However, epegRNAs that template transversions at RNF2 or EMX1 were expressed to greater levels than those of unmodified pegRNA (2.2- to 2.4-fold and 1.4- to 3.7-fold, respectively, FIGS. 114A-114C). These data suggest that the 3′ motifs affect pegRNA expression inconsistently, concordant with the earlier finding (FIG. 104B) that PE efficiency under these transfection conditions is not limited by pegRNA expression in HEK293T cells. When epegRNA expression is more limiting, however, improving epegRNA expression might further improve editing efficiency.

Next, it was tested if the addition of a 3′ RNA structural motif reduced engagement of the target DNA site by comparing the ability of epegRNAs and pegRNAs to support transcriptional activation by dCas9-VP64-p65-Rta (dCas9-VPR) fusions was tested^{32,33} HEK293T cells were transfected with plasmids encoding dCas9-VPR, GFP downstream of either the HEK3, DNMT1, RNF2, or EMX1 target protospacer, and either pegRNAs, epegRNAs, or sgRNAs targeting the corresponding site. Transcriptional activation was measured via cellular GFP fluorescence after three days. In contrast to their ability to enhance PE activity (FIG. 105A), epegRNAs showed similar Cas9-dependent transcriptional activation in HEK293T cells as pegRNAs (FIG. 106F). Both epegRNAs and canonical pegRNAs resulted in lower transcriptional activation compared to an sgRNA targeting the same site (3.0-fold for pegRNA, 2.3-fold for evopreQ1 epegRNA, and 1.9-fold for mpknot epegRNA across four sites), suggesting that the 3′ extension in pegRNAs and epegRNAs modestly impedes target site engagement.

To deconvolute potential changes in target site engagement and differences in pegRNA and epegRNA expression, microscale thermophoresis (MST) was performed to measure the affinity of pre-incubated RNP complexes of catalytically inert Cas9 (dCas9) and pegRNAs or epegRNAs for a dsDNA substrate. It was found that addition of mpknot or evopreQ1 resulted in comparable or modestly reduced binding affinity for dsDNA compared to unmodified pegRNA respectively (K_{D}=10 nM for evopreQ1 epegRNA and 21 nM for mpknot pegRNA versus 8.1 nM for unmodified pegRNA, FIG. 106E). Affinity of pegRNAs for Cas9 H840A nickase was also modestly reduced by either motif (K_{D}=18 nM for evopreQ1 epegRNA, 11 nM for mpknot pegRNA, and 5 nM for unmodified pegRNA; FIG. 106G). These findings suggest that increased PE efficiency from epegRNAs does not arise from improved binding of the pegRNA to Cas9, or of the PE RNP complex to the targeted site.

Taken together, these results suggest that epegRNAs are more resistant to cellular degradation than pegRNAs and thus generate fewer truncated pegRNA species that erode prime editing efficiency. Additional mechanisms behind improvements from epegRNAs cannot be excluded.

Optimization of Engineered pegRNA 3′ Motifs

Having established that epegRNAs improve editing efficiency by resisting exonucleolytic degradation, it was hypothesized that more stable RNA motifs canmight further improve PE activity. Twenty-five additional structured RNA motifs were screened for their ability to improve epegRNA editing efficiency across epegRNAs encoding either the installation of a 24-bp FLAG epitope tag insertion, a 15-bp deletion, or a transversion at HEK3 or RNF2, were examined (FIGS. 116A-116D, FIGS. 117A-117C). These motifs included additional evolved prequeosine₁-1 riboswitch aptamers²¹, mpknot variants with improved pseudoknot stability²², G-quadruplexes of increasing stability³⁴, 15-bp hairpins, an xrRNA³⁵, and the P4-P6 domain of the group I intron³⁶. While 123 of the 137 epegRNAs tested exhibited improved overall prime editing compared to the corresponding pegRNAs, none demonstrated consistent improvements over evopreQ₁ or mpknot across the majority of edits tested (FIGS. 116A-116D, FIGS. 117A-117C).

Next, trimming unnecessary sequence from the added evopreQ₁ and mpknot motifs can further improve the epegRNA design because removing extraneous sequences within a structured RNA can reduce the propensity for misfolding³⁷. It was found that trimming 5 nt of excess sequence from evopreQ1 or mpknot resulted in marginal gains in average PE3-editing efficiency relative to the full-length epegRNAs (FIGS. 117A-117C). Since trimming these RNA motifs did not adversely affect editing efficiency and shorter epegRNAs are more readily prepared by chemical synthesis, trimmed evopreQ₁ (tevopreQ₁) was used in epegRNAs when applying epegRNAs to install therapeutically relevant mutations (see below).

It was also examined whether the “flip and extension” (F+E) sgRNA scaffold³⁸ would further improve epegRNA editing efficiency. This guide RNA scaffold mutates the fourth base pair of the direct repeat from U•A to A•U to remove a potential pol III terminator and extends the direct repeat by five base pairs to improve Cas9 binding³⁸. HEK293T cells were transduced with lentiviruses encoding either an unmodified (F+E) pegRNA, an (F+E) epegRNA containing tevopreQ1, or a tevopreQ1 epegRNA with the standard scaffold that templates a transversion at HEK3 or DNMT1, or a 3-nt insertion at HEK3. Use of tevopreQ1 substantially improved editing efficiency (3.8-fold for the nucleotide transversion and 2.6-fold for the 3-nt insertion at HEK3 and 6.8-fold at DNMT1) (FIG. 118). Use of the (F+E) scaffold in a tevopreQ1 epegRNA further improved editing efficiency (1.1-fold for the nucleotide transversion, 1.5-fold for the 3-nt insertion at HEK3, and 2.5-fold at DNMT1). sgRNA scaffold variants previously shown to increase Cas9-nuclease activity³⁹ under transfection conditions with reduced amounts of plasmid were also characterized, and similar overall benefits were observed, albeit with greater variability (FIG. 119). These findings further suggest that epegRNAs mediate greater improvements in PE efficiency when expression is limited. Additionally, these data highlight the potential for modified scaffolds to improve PE efficiency in conjunction with epegRNAs.

A Computational Tool to Design epegRNA Linkers

In contrast with protein linkers, RNA linkers more likely to be sequence-dependent, such that the same linker might function for one epegRNA but impede another. To minimize the possibility of interference from the epegRNA linker, pegLIT (pegRNA Linker Identification Tool) was developed (FIGS. 120A-120F), a computational tool that identifies linker sequences predicted to minimally base pair with the remainder of the epegRNA. For an initial validation, two sets of 15 evopreQ1 epegRNAs were tested with different linkers templating either a C•G-to-A•T transversion at RNF2 or a 15-bp deletion at DNMT1. Within each set, five linkers were recommended by pegLIT; five were predicted to base pair with the spacer, and five were predicted to base pair with the PBS. The use of pegLIT-designed linkers resulted in a modest increase in PE3 editing efficiency over the use of manually designed linkers (1.2-fold higher for RNF2 and 1.1-fold higher for DNMT1) (FIGS. 120A-120F). While spacer interactions did not significantly impact editing efficiency, linker-PBS interactions correlated with reduced PE3-editing efficiency, resulting in 1.3- and 1.1-fold lower editing efficiency compared to pegLIT linkers for RNF2 and DNMT1 respectively. The two worst-performing linkers, which resulted in 1.9- and 3.4-fold less efficient PE3 editing at RNF2 relative to optimal linker sequences, were correctly identified by pegLIT as scoring poorly for PBS interactions (FIGS. 120A-120F). The closer proximity of the linker to the PBS compared to the spacer may give linker:PBS interactions an entropic advantage compared to linker:spacer pairing.

PegLIT-designed linker sequences were studied to determine whether they could improve the efficacy of two epegRNAs (templating a G•C-to-T•A transversion at EMX1 and a 15-bp deletion at VEGFA) which initially failed to exhibit improved editing (FIGS. 111A-111K). Indeed, using pegLIT-designed linkers increased PE3 editing efficiency by 1.3-fold and 1.4-fold, respectively, over that of pegRNAs for these two edits (FIGS. 120A-120G). Collectively, these findings demonstrate that pegLIT facilitates the use of epegRNAs to consistently improve prime editing outcomes.

PegLIT-designed linkers were also studied to determine whether they improved the activity of epegRNAs compared to epegRNAs without linkers. Compared to mpknot epegRNAs without a linker, adding a pegLIT-designed linker resulted in a slightly increased editing efficiency than when using manually designed linkers (FIGS. 110 and 120A-120F). In contrast, the use of pegLIT linkers with evopreQ1 or tevopreQ1 epegRNAs did not significantly increase editing relative to epegRNAs without a linker (FIGS. 120A-120G).

Improved Editing Efficiency with Chemically Modified epegRNAs

Chemically synthesized gRNAs are commonly used when transfecting cells with mRNA or RNPs⁴⁰. Although synthetic gRNAs can incorporate chemical modifications that promote resistance to exonucleolytic-degradation^{16,40}, it was considered speculated that structural motifs might still mediate additional improvements in conjunction with such modification.

To demonstrate this possibility, prime editing efficiencies of synthetic tevopreQ₁ epegRNAs were compared with those of synthetic pegRNAs that install either a point mutation or 15-bp deletion at five genomic sites (HEK3, RNF2, DNMT1, RUNX1, and EMX1) in HEK293T cells. Both the epegRNAs and pegRNAs contained 2′-O-methyl modifications and phosphorothioate linkages between the first and last three nucleotides of the RNA. For six of the seven pegRNAs tested, the corresponding epegRNAs exhibited 1.1- to 3.1-fold higher editing with unchanged edit:indel ratios (FIGS. 121A-121B). These data suggest that epegRNAs also enhance PE outcomes compared to pegRNAs in applications that use chemically synthesized and modified pegRNAs.

Engineered pegRNAs Improve Prime Editing of Therapeutically Relevant Mutations

Having validated the use of epegRNAs as a strategy for broadly improving PE activity, we next compared the activity of epegRNAs containing tevopreQ₁ with that of pegRNAs to install a variety of protective or therapeutic genetic mutations. epegRNAs were successfully used to install the PRNP G127V allele that protects against human prion disease^{41,42} in HEK293T cells with 1.4-fold higher efficiency over the canonical pegRNA (FIG. 107A). In addition, epegRNAs were used to correct the most common cause of Tay-Sachs disease (HEXA^{1278+TATC}), both in previously constructed HEXA^{1278+TATC} HEK293T cell lines¹⁴ via plasmid lipofection and in primary patient-derived fibroblasts via nucleofection of in vitro transcribed mRNA and synthetic pegRNA (FIGS. 107B-107C). In both cases, improved editing efficiencies were observed for tevopreQ1epegRNAs containing pegLIT-designed 8-nt linkers over canonical pegRNAs (2.8-fold higher in HEK293T cells and 2.3-fold higher in patient-derived fibroblasts).

Installation of Therapeutically Relevant Edits Using Unoptimized epegRNAs

The design and screening of many pegRNAs with different PBS and RT templates is an important first step in the successful use of prime editing¹⁴. Although general rules to guide PBS and RT template length and composition have been described^{14,43}, identifying optimal pegRNAs often requires extensive screening of pegRNA constructs. It was considered that epegRNAs can support more efficient installation of therapeutically relevant prime edits even without extensive pegRNA optimization. The ability of unoptimized pegRNAs and epegRNAs to template the installation of nine protective or pathogenic point mutations using PE2. In all cases, the pegRNAs and epegRNAs used in this experiment contained a 13-nt PBS and an RT template containing 10 nt of homology to the targeted site after the last edited nucleotide, except when the 3′ extension would begin with cytosine¹⁴, in which case it was extended to the nearest non-C nucleotide.

pegRNAs that install therapeutically relevant mutations associated with Alzheimer's disease⁴⁴, coronary heart disease^{45,46}, type-2 diabetes⁴⁷, innate immunity⁴⁸, CDKL5 deficiency disorder⁴⁹, lamin A deficiency⁵⁰, and Rett syndrome^{51,52} were examined. These nine mutations include protective alleles in APP, PCSK9, SLC30A8, CD209, and CETP, as well as pathogenic mutations in CDKL5, LMNA, and MECP2⁵⁴. The outcomes of prime editing with pegRNAs and corresponding tevopreQ1 epegRNAs with 8-nt pegLIT linkers in HEK293T cells (FIG. 111D) were compared. Only a single pegRNA or epegRNA design was tested per target. In every case, epegRNAs outperformed pegRNAs in editing efficiency. For five of the nine therapeutically relevant edits tested, epegRNAs resulted in ≥20% editing efficiency, which is typically sufficient to generate model cell lines. By comparison, only three of the nine pegRNAs achieved this level of editing efficiency. The higher editing efficiencies mediated by epegRNAs (2.8-fold higher than pegRNAs on average) should streamline the production of homozygous cell lines, an important consideration for modeling recessive mutations. Similarly, unoptimized epegRNAs mediated insertion of a 24-bp FLAG tag with ≥10% efficiency at 5 of 15 tested sites; the corresponding pegRNAs did not achieve ≥10% efficiency at any site tested (FIGS. 122A-122B). Taken together, these findings demonstrate that epegRNAs streamline the production of model cell lines with PE.

### Discussion

Presented herein are the design, characterization, and validation of engineered pegRNAs to address a key bottleneck in prime editing. These epegRNAs contain a structured RNA motif 3′ of the PBS that prevents degradation of the pegRNA extension and the subsequent formation of editing-incompetent PE complexes that compete for access to the targeted genomic site. It was found that epegRNAs broadly improve prime editing efficiency in all five cell lines and primary cell types tested, with larger improvements observed in cell lines that are more difficult to transfect. Additionally, it was observed that the use of epegRNAs can enhance prime editing performance when using chemically modified pegRNAs, when installing therapeutically relevant edits in human cells, and when using unoptimized pegRNA designs. Finally, a computational program that expedites epegRNA design by identifying linkers that minimize the risk of counterproductive secondary structure was described. In total, these findings establish that epegRNAs broadly improve prime editing outcomes at a wide variety of genomic loci, edit types (substitutions, insertions, and deletions), and cell types.

Improvements in prime editing enabled by epegRNAs are likely to depend on delivery strategy. Lower-expression delivery modalities such as some viral vectors might benefit more strongly from the use of epegRNAs when pegRNA concentration is limiting (FIG. 119). Similarly, further improvements in the synthesis of chemically modified RNAs might decrease the benefits of epegRNAs by mitigating pegRNA 3′ degradation. Additionally, the longer length of epegRNAs (an additional 37 nt when using tevopreQ₁) is an important consideration when using synthetic epegRNAs given current challenges of chemically synthesizing longer RNAs.

The use of epegRNAs is recommended for prime editing experiments that can support a modestly longer pegRNA. Extensive screening may not be needed when maximizing editing efficiency is not the priority. In these cases, an epegRNA containing the trimmed evopreQ₁ motif and an 8-nt pegLIT-designed linker with a PBS length of 13 and a template that includes either 10 nt of homology past the targeted edit for small insertions, deletions, and point mutations-or 25 nt of homology for larger insertions or deletions-provides a promising starting point for epegRNA designs. PBS, RT template length, and nicking sgRNA can then be optimized if observed editing efficiencies are insufficient.

pegLIT Strategy for Identifying Optimal Linker Sequences

pegLIT uses simulated annealing to sample the analyzed linker space efficiently¹. Linkers that are adenosine- or cytosine-rich are preferred by pegLIT since these nucleotides have been reported to function better as flexible RNA linkers². Additionally, pegLIT filters out linkers that contain runs of four or more uridines, since such sequences could cause premature transcriptional termination³.

The pegLIT tool then analyzes linkers that pass these requirements using ViennaRNA⁴ to predict potential interactions between the linker sequence and the pegRNA spacer, PBS, template, or scaffold. The base pair probabilities of these predicted interactions are used to generate subscores for each region of the pegRNA, each of which represents the degree to which the linker is predicted to avoid interaction with the associated region. For example, a subscore of 0.95 for the PBS essentially indicates that, on average, the predicted probability of a pegRNA folded state lacking base pairing between any linker nucleotide and the PBS is 95%.

The use of pegLIT was validated for linker design and which interactions identified by pegLIT were most detrimental to editing efficiency was examined. 30 linker sequences were generated (10 recommended by pegLIT, 10 interacting with the spacer, and 10 interacting with the PBS) to test with evopreQ₁ epegRNAs templating either a C•G-to-A•T transversion at RNF2 or a 15-bp deletion at DNMT1. The average spacer and PBS subscores were 0.94 and 0.97 for the optimal sequences, 0.66 and 0.95 for the spacer sequences, and 0.86 and 0.21 for the PBS sequences. Relative to the recommended designs, use of the PBS-interacting linkers was associated with 1.3- and 1.1-fold lower editing efficiency at RNF2 and DNMT1 respectively (FIGS. 120A-120G), whereas the spacer-interacting linkers had a negligible effect on editing efficiency. This difference may be because the closer proximity of the linker to the PBS compared to the spacer may give linker:PBS interactions an entropic advantage compared to linker:spacer pairing.

epegRNAs Delivered Via Plasmid Transfection with Optimized Guide RNA Scaffolds in HEK293T Cells

To mimic lower expression conditions, HEK293T cells were transfected with 20 ng of PE2 plasmid and 4 ng of pegRNA or epegRNA plasmid when assessing the applicability of “flip and extension” (F+E) sgRNA scaffold variants for PE. The editing efficiency of epegRNAs targeted to PRNP, HEK3, RUNX1, and EMX1 that contained the canonical sgRNA scaffold, an (F+E) scaffold⁵, or one of six (F+E) scaffolds bearing mutations previously shown to increase Cas9-nuclease activity⁶ were compared. It was found that these alternative scaffolds overall either maintained or improved PE efficiency relative to the standard scaffold, with cr772 exhibiting the best improvement (FIG. 119). While efficiency improvements were less consistent under these conditions compared to lentiviral transduction (FIG. 118), this may stem from differences in expression. EpegRNA expression is likely several-fold higher following plasmid transfection than that following single-copy lentiviral transduction, which may partially obfuscate the benefits of more efficient transcription and Cas9 binding affinity. Testing cr772 or the original (F+E) scaffold to further improve PE efficiency with epegRNAs is recommended, especially for applications with lower expression than plasmid transfection.

Installation of FLAG Tags Using Unoptimized epegRNAs

epegRNAs and pegRNAs were compared for the installation of more challenging edits, such as insertion of the 24-bp FLAG epitope tag (FIG. 105A). The ability of unoptimized pegRNAs and tevopreQ₁ epegRNAs containing one of two loci-specific pegLIT-designed 8-nt linkers to template the installation of a FLAG epitope tag at 15 loci in HEK293T cells using PE2 was assessed (FIGS. 122A-122B). The unoptimized epegRNAs and pegRNAs were designed with a 13-nt PBS and an RT template containing 25 nt of homology downstream of the inserted FLAG epitope tag, except when the 3′ extension would begin with cytosine7, in which case it was extended to the nearest non-C nucleotide. The use of epegRNAs enabled FLAG tags to be installed with PE2 at ≥10% efficiency with no PBS and RT template optimization at 5 of the 15 sites, while ≥10% efficiency was not observed with any pegRNAs (FIGS. 122A-122B). These observations further demonstrate that epegRNAs can enhance prime editing performance for a variety of edits at many different endogenous human genomic loci.

### Methods

General Methods. Plasmids expressing pegRNAs and epegRNAs were cloned either by Gibson assembly, Golden Gate assembly using either a previously described custom acceptor plasmid¹⁴ or newly designed custom acceptor plasmids that contain trimmed evopreQ₁ or mpknot (the use of which is described below), or they were synthesized and cloned by Twist Biosciences. Plasmids expressing sgRNAs were cloned via Gibson or USER assembly. DNA amplification was accomplished by PCR with Phusion U or High Fidelity Phusion Green Hot Start II (New England Biolabs). Plasmids expressing pegRNAs were purified using PureYield plasmid miniprep kits (Promega) when transfecting HEK293T cells or Plasmid Plus Midiprep kits (Qiagen) when transfecting other cell types, while plasmids expressing prime editors were purified exclusively using Plasmid Plus Midiprep kits. Plasmids ordered from Twist Biosciences were resuspended in nuclease-free water and used directly. Primers and dsDNA fragments were ordered from Integrated DNA Technologies (IDT).

Guidelines for epegRNA cloning via Golden Gate DNA assembly⁶¹. When cloning epegRNAs using the Golden Gate method, the same protocol as previously described¹⁴ is appropriate with the important note that the junction sequence between the 3′ extension oligo and the plasmid backbone is different for epegRNAs using tevopreQ₁ and trimmed mpknot (tmpknot), as shown below. More details on pegRNA design and cloning are available at liugroup.us. Plasmid backbones used for Golden Gate cloning have been deposited with Addgene. SEQ ID NOs: 486-489 (top-bottom):

| | |
|---|---|
| Forward oligo for | 5′-GTGCNNNNNNNNNNNN |
| 3′ extension of | NNNNNNNNNNNN -3' |
| pegRNAs and | |
| epegRNAs | |
| Reverse oligo for | 3′- NNNNNNNNNNNN |
| 3′ extension | NNNNNNNNNNNNAAAA-5′ |
| of pegRNAs | |
| Reverse oligo for | 3′- NNNNNNNNNNNN |
| 3′ extension of | NNNNNNNNNNNNGCGC-5′ |
| epegRNAs with | |
| tevopreq, −1 | |
| Reverse oligo for | 3′- NNNNNNNNNNNN |
| 3′ extension | NNNNNNNNNNNNGGGAGTC-5′ |
| of epegRNAs | |
| with tmpknot | |

Synthetic pegRNAs and in vitro transcribed mRNA generation. Synthetic pegRNAs were ordered from IDT and contained 2′-O-methyl modifications at the first and last three nucleotides and phosphorothioate linkages between the three first and last nucleotides and were used directly. Synthetic nicking sgRNAs were ordered from Synthego and contained 2′-O-methyl modifications at the three first and last nucleotides and phosphorothioate linkages between the first three and last two nucleotides. PE-encoded mRNA was transcribed in vitro using the protocol in Gaudelli et al. (2020). Briefly, the PE2 cassette—consisting of a 5′ UTR, Kozak sequence, PE2 ORF and 3′ UTR—was cloned into a plasmid containing an inactive T7 (dT7) promoter. The mRNA transcription template was generated via PCR using a primer to install the correct T7 promoter sequence and a reverse primer which installed the poly-A tail. mRNA was generated using a HiScribe T7 High-Yield RNA Kit (New England Biolabs) according to the manufacturer's instructions, with the exception that N1-methylpseudouridine triphosphate (Trilink) was substituted for uridine triphosphate and CleanCapAG (Trilink) was added to enable co-transcriptional capping. The resulting mRNA was purified via lithium chloride precipitation and reconstituted in TE buffer (10 mM Tris, 1 mM EDTA, pH 8.0 at 25° C.). Sequences of pegRNAs and sgRNAs used in this example can be found in Table E1. A list of structured RNA motifs examined in this example can be found in Table E2.

General mammalian cell culture conditions. HEK293T (ATCC CRL-3216), U20S (ATCC HTB-96), K562 (CCL-243), and HeLa (CCL-2) cells were purchased from ATCC and cultured and passaged in Dulbecco's Modified Eagle's Medium (DMEM) supplemented with GlutaMax (Thermo Fisher Scientific), McCoy's 5A Medium (Gibco), RPMI Medium 1640 plus GlutaMAX (Gibco), or Eagle's Minimal Essential Medium (EMEM, ATCC), respectively, each supplemented with 10% (v/v) fetal bovine serum (Gibco, qualified). Primary Tay Sachs disease patient fibroblast cells were obtained from the Coriell Institute (Cat. ID GM00221) and grown in low-glucose DMEM (Sigma Aldrich) and 10% (v/v) FBS, supplemented with an additional 2 mM L-glutamine (Thermo Fisher Scientific). All cell types were incubated, maintained, and cultured at 37° C. with 5% CO2. Each cell line was authenticated by its respective supplier and tested negative for mycoplasma.

Tissue culture transfection and nucleofection protocols and genomic DNA preparation. For transfections, 10,000 HEK293T cells were seeded per well on 96-well plates (Corning). 16-24 hours post-seeding, cells were transfected at approximately 60% confluency with 0.5 μL of Lipofectamine 2000 (Thermo Fisher Scientific) according to the manufacturer's protocols and 200 ng of PE plasmid, 40 ng of pegRNA plasmid, and 13 ng of sgRNA plasmid (for PE3).

For nucleofections, HEK293T cells were electroporated with in vitro transcribed mRNA and synthetic pegRNA using a Lonza 4D Nucleofector with an SF cell line kit (Lonza). 200,000 cells per electroporation were centrifuged for 8 min at 120×g, then washed in 1 mL PBS (Thermo Fisher Scientific). After a second centrifugation, cells were resuspended in 5 μL reconstituted SF buffer per sample and added to microcuvettes.

For each cuvette, 17 μL of cargo mix (1 ug of PE2 mRNA in 0.5 μL, 90 pmol of pegRNA in 0.9 μL, and 60 pmol of nicking sgRNA in 0.6 μL, and 15 μL of reconstituted SF buffer) was added and pipetted up and down three times to mix. Cells were electroporated using program CM-130, then 80 μL of warm media was added and cells were incubated for 10 min at room temperature. The mixture was then pipetted to mix and 25 μL was added to the well of a 48-well plate, with a final culture volume of 250 μL per well. For experiments in HeLa, U20S, and K562 cells, 800 ng PE2-expressing plasmid, 200 ng pegRNA-expressing plasmid, and 83 ng nicking sgRNA-expressing plasmid were nucleofected in a final volume of 20 μL in a 16-well nucleovette strip (Lonza). HeLa cells were nucleofected using the SE Cell Line 4D-Nucleofector X Kit (Lonza) with 2×10⁵ cells per sample (program CN-114), according to the manufacturer's protocol. U20S cells were nucleofected using the SE Cell Line 4D-Nucleofector X Kit (Lonza) with 2×10⁵ cells per sample (program DN-100), according to the manufacturer's protocol. K562 cells were nucleofected using the SE Cell Line 4D-Nucleofector X Kit (Lonza) with 2×10⁵ cells per sample (program FF-120), according to the manufacturer's protocol.

Patient-derived fibroblasts were electroporated with mRNA-encoding PE2 and synthetic pegRNA and nicking sgRNA as described above for HEK293T cells using an SE cell line kit and 100,000 cells which were centrifuged at 100×g for 10 min. Additionally, 40 μL of recovered cells were added to a 48 well plate instead of 25. In all cases, cells were cultured 3 days following transfection, after which the media was removed, and cells were washed with PBS and subsequently lysed by the addition of 50 μL for 96-well plates or 150 μL for 48-well plates of freshly prepared lysis buffer (10 mM Tris-HCl, pH 8 at 25° C.; 0.05% SDS; 25 μg mL⁻¹ Proteinase K (Qiagen)), and incubating at 37° C. for 1 hour or more, after which Proteinase K was inactivated over 30 minutes at 80° C. The resulting gDNA was stored at −20° C. until used.

High-throughput DNA sequencing of genomic DNA samples. Genomic sites of interest were amplified from genomic DNA samples and sequenced on an Illumina MiSeq as previously described¹⁴. Cas9 off-target sites for HEK3, EMX1, and FANCF were previously identified via Guide-Seq²⁹. Primers used for mammalian cell genomic DNA amplification are listed in Table E3 and amplicons are listed in Table E4. Sequencing reads were demultiplexed using MiSeq Reporter (Illumina). Alignment of amplicon sequences to a reference sequence was performed using CRISPResso2⁵⁹. For all prime editing yield quantifications, editing efficiency was calculated as the percentage of reads with the desired editing without indels out of the total number of reads with an average phred score of at least thirty. For quantification of point mutation editing, CRISPResso2 was run in standard mode with “discard_indel_reads” on. Editing yield was calculated as the percentage of non-discarded reads containing the edit divided by total reads. For insertion or deletion edits, CRISPResso2 was run in HDR mode using the desired allele as the expected allele, and with “discard_indel_reads” on. Editing yield was calculated as the percentage of HDR aligned reads divided by total reads. For all experiments, indel frequency was calculated as the number of discarded reads divided by the total number of reads. For experiments involving PE2, reads were analyzed for indels within 10 nucleotides up- and downstream of the pegRNA nick site, inclusive. For experiments involving PE3, reads were analyzed for indels between 10 nucleotides upstream of the pegRNA nick site and downstream from the sgRNA nick site, inclusive. Off-target editing was quantified as described previously¹⁴.

In vitro exonuclease susceptibility assays, pegRNAs or epegRNAs containing either mpknot or evopreQ₁ were prepared using the HiScribe T7 Quick High Yield RNA synthesis kit (New England Biolabs) from PCR-amplified templates containing a T7 promoter sequence per the manufacturer's protocols. Nuclear extracts were prepared from 3 million HEK293T cells grown to 70-80% confluency per the manufacturer's protocols using the EpiQuik Nuclear Extraction kit (EpiGentek). Assays were carried out in 10 μL reactions containing 20 mM Tris-HCl (pH 7.5), 5 mM MgCl2, 50 mM NaCl, 2 mM DTT, 1 mM NTP and 0.8 U/l RNaseOUT Recombinant Ribonuclease Inhibitor (40 U/L; ThermoFisher Scientific) to inhibit endonuclease activity. 3 μL of fresh nuclear lysate was used to degrade 0.5 μg of RNA substrate per reaction. Followed by the incubation of reaction mixtures at 37° C. for 20 min, degradation products were resolved on 2.0% agarose gels stained with SYBR Gold. The extent of degradation was determined using ImageJ software (NIH).

RTqPCR of total RNA. 10,000 HEK293T cells per well were seeded in 96-well plates. 16-24 hours post-seeding, cells were transfected at approximately 60% confluency with 0.5 μL of Lipofectamine 2000 according to the manufacturer's protocols and 200 ng of PE2 plasmid and 40 ng of either pegRNA or epegRNA plasmid. After three days, the Power SYBR Green Cells-to-C_{T} kit (Thermo Fisher Scientific) was used to extract total RNA, to reverse transcribe total cDNA with random hexamers, and to perform qPCR with forward and reverse primers that amplify the sgRNA scaffold, according to the manufacturer's protocols. Primer sequences are available in Table E5.

Cas9-based transcriptional activation. 10,000 HEK293T cells per well were seeded in 96-well black-wall plates (Corning). 16-24 hours post-seeding, cells were transfected at approximately 60% confluency with 0.5 μL of Lipofectamine 2000 according to the manufacturer's protocols and 100 ng of dCas9-VPR plasmid, 30 ng of GFP reporter plasmid, 15 ng of iRFP plasmid, and 20 ng of sgRNA, pegRNA, or epegRNA plasmid. After three days, cells were measured for GFP and iRFP fluorescence using an Infinite M1000 Pro microplate reader (Tecan). GFP fluorescence was normalized to iRFP fluorescence after subtracting background fluorescence signal from untreated cells.

Linker design via pegLIT. To design epegRNA linker sequences, a custom algorithm, pegRNA Linker Identification Tool, or pegLIT, was written that searches for linker sequences of a specified length that minimize base pairing with the remainder of the pegRNA. This procedure uses simulated annealing to maximize subscores, each of which corresponds to a subsequence of the pegRNA: spacer, PBS, template, or scaffold. During optimization, the higher-scoring linker in any pair of linkers was determined by comparing their discretized subscores in order of the following subsequence priority: spacer, PBS, template, and then scaffold. Each subscore is calculated, using base pair probabilities calculated by ViennaRNA 2.0²⁵ under standard parameters (37° C., 1 M NaCl, 0.05 M MgCl2), as the complement of the mean probability that a nucleotide in the linker forms a base pair with any nucleotide in the pegRNA subsequence under consideration, where the mean is taken over all bases in the linker. Linker sequences with AC content <50% and those that would result in a pegRNA containing four of the same nucleotide consecutively are removed from consideration^{39,40}. Optionally, the algorithm performs hierarchical agglomerative clustering on the 100 highest-scoring linkers and outputs one linker per cluster in order to promote sequence diversity in the final output. The code for pegLITis shown below:

```
            from math import prod
            from random import choice, randint, random
            import heapq
            import numpy as np
            from tqdm import trange
            from scipy.special import expit as sigmoid
            from sklearn.cluster import AgglomerativeClustering as HAC
            from Levenshtein import distance as levenshtein_distance
            import RNA
            BASE_SYMBOLS = {
              “A”: (“A”,), “C”: (“C”,), “G”: (“G”,), “T”: (“T”,), “U”: (“T”,),
              “W”: (“A”, “T”), “S”: (“C”, “G”), “M”: (“A”, “C”),
              “K”: (“G”, “T”), “R”: (“A”, “G”), “Y”: (“C”, “T”),
              “B”: (“C”, “G”, “T”), “D”: (“A”, “G”, “T”), “H”: (“A”, “C”, “T”), “V”: (“A”, “C”,
            “G”),
              “N”: (“A”, “C”, “G”, “T”)}
            def apply_filters(seq_pre, seq_linker, seq_post, ac_thresh, u_thresh, n_thresh):
              ″″″
              Returns False if any filter is failed i.e. AC content < ac_thresh OR consecutive Us >
            u_thresh.
              OR consecutive Ns > n_thresh. Otherwise, True if all filters are passed. All
            thresholds have
              units nt (i.e. ac_thresh is not a percent). Ts are treated as Us.
              ″″″
              # AC content
              if seq_linker.count(“A”) + seq_linker.count(“C”) < ac_thresh:
                return False
              # Consecutive U
              seq_neighborhood = seq_pre[-(u_thresh):] + seq_linker + seq_post[:u_thresh]
              seq_neighborhood = seq_neighborhood.replace(“T”, “U”)
              if “U” * (u_thresh + 1) in seq_neighborhood:
                return False
              # Consecutive N
              seq_neighborhood = seq_pre[-(n_thresh):] + seq_linker + seq_post[:n_thresh]
              seq_neighborhood = seq_neighborhood.replace(“T”, “U”)
              if any(nt * (n_thresh + 1) in seq_neighborhood for nt in set(seq_linker)):
                return False
              return True
            def calc_subscores(linker_pos, *sequence_components):
              ″″″
              Calculate base-pairing probs marginalized for each nucleotide
              ″″″
              # Calculate bpp from ViennaRNA
              pegrna = RNA.fold_compound(“”.join(sequence_components))
              _ = pegrna.pf( ) # need to first internally calculate partition function
              basepair_probs = np.array(pegrna.bpp( ))[1:, 1:]
              # Fill in lower-triangle and diagonal of ViennaRNA's upper-triangular bpp matrix
              unpaired_probs = 1. - (basepair_probs.sum(axis=0) + basepair_probs.sum(axis=1))
              # copy data to make symmetric
              i_lower = np.tril_indices(len(basepair_probs), -1)
              i_diag = np.eye(len(basepair_probs), dtype=bool)
              basepair_probs[i_lower] = basepair_probs.T[i_lower]
              basepair_probs[i_diag] = unpaired_probs
              # Track indices of subsequences
              idx_cur = 0
              seq_idx = [ ]
              for subseq in sequence_components:
                idx_prev = idx_cur
                idx_cur += len(subseq)
                seq_idx.append(slice(idx_prev, idx_cur))
              # Extract subscores for subsequences
              bpp_subseq = np.ma.masked_all(len(sequence_components))
              for i, subseq in enumerate(sequence_components):
                bpp_within_subseq = basepair_probs[seq_idx[i], seq_idx[linker_pos]]
                bpp_subseq[i] = np.mean(np.sum(bpp_within_subseq, axis=0))
              return bpp_subseq
            def apply_score(seq_spacer, seq_scaffold, seq_template, seq_pbs, seq_linker,
                    score_to_beat=None, epsilon=0.01):
              ″″″
              Calculates subscores then outputs hashed score. Terminates calculation early if score
            will
              be less than score_to_beat. Prioritize PBS, spacer, template, scaffold.
              ″″″
              # Cas9 complex at R loop subscore
              bpp_subseq1 = calc_subscores(2, seq_template, seq_pbs, seq_linker)
              subscore_pbs = 1. - bpp_subseq1[1]
              subscore_template = 1. - bpp_subseq1[0]
              # Free pegRNA subscore
              if ((score_to_beat is not None)
                  and (epsilon * int(subscore_pbs / epsilon) < score_to_beat[0])):
                subscore_spacer = 0.
                subscore_scaffold = 0.
              else:
                bpp_subseq2 = calc_subscores(4, seq_spacer, seq_scaffold,
                              seq_template, seq_pbs, seq_linker)
                subscore_spacer = 1. - bpp_subseq2[0]
                subscore_scaffold = 1. - bpp_subseq2[1]
              # Turn subscores into a single score
              return tuple(
                epsilon * int(val / epsilon)
                if val is not None else 0
                for val in (subscore_pbs, subscore_spacer, subscore_template, subscore_scaffold)
                )
            def optimize(seq_spacer, seq_scaffold, seq_template, seq_pbs, seq_motif,
                  linker_pattern, ac_thresh, u_thresh, n_thresh, topn, epsilon,
                  num_repeats, num_steps, temp_init, temp_decay):
              ″″″
              Simulated annealing optimization of linkers
              ″″″
              ## Pre-process inputs
              seq_pre = seq_spacer + seq_scaffold + seq_template + seq_pbs
              seq_post = seq_motif
              ac_thresh = ac_thresh * len(linker_pattern)
              ## Simulated annealing to optimize linker sequence
              # Initialize hashmap of sequences already considered
              linker_skip = { }
              len_sequence_space = prod(len(BASE_SYMBOLS[nt]) for nt in linker_pattern)
              # Initialize min heap of topn linkers
              linker_heap = [ ]
              for _ in trange(num_repeats, desc=“Repeats”, position=0, leave=False):
                # Initialize simulated annealing
                seq_linker_prev = “”.join([choice(BASE_SYMBOLS[nt]) for nt in linker_pattern])
                score_prev = None
                temp = temp_init
                for _ in trange(num_steps, desc=“Steps”, position=1, leave=False):
                  # Generate new sequence by substituting characters in sequence until pass filters
                  seq_linker = seq_linker_prev
                  keep_going = True
                  while keep_going:
                    char_pos = randint(0, len(linker_pattern) - 1)
                    seq_linker = (
                      seq_linker[:char_pos]
                      + choice(BASE_SYMBOLS[linker_pattern[char_pos]])
                      + seq_linker[(char_pos + 1):])
                    keep_going = (
                      seq_linker in linker_skip
                      or not apply_filters(seq_pre, seq_linker, seq_post,
                                ac_thresh, u_thresh, n_thresh)
                      or len(linker_skip) >= len_sequence_space) # already screened whole seq space
                    linker_skip[seq_linker] = True
                  # Calculate score for linker sequence
                  score_to_beat = linker_heap[0][0] if len(linker_heap) >= topn else None
                  score = apply_score(seq_spacer, seq_scaffold, seq_template, seq_pbs, seq_linker,
                            score_to_beat=score_to_beat, epsilon=epsilon)
                  # Add to min heap i.e. maintains the top `topn` largest entries
                  if score_to_beat is None: # heap is not yet full
                    heapq.heappush(linker_heap, (score, seq_linker))
                  elif score > score_to_beat:
                    heapq.heapreplace(linker_heap, (score, seq_linker))
                  # Decide if keep proposal
                  if (score_prev is None  # initialize
                    or score > score_prev  # exploit improvement
                    or random( ) < sigmoid(  # explore
                      sum((s1 - s2) * (epsilon ** i)
                        for i, (s1, s2) in enumerate(zip(score, score_prev))) / temp
                      )):
                    seq_linker_prev = seq_linker
                    score_prev = score
                  # Update simulated annealing param
                  temp *= temp_decay
              linker_heap_scores, linker_heap = zip(*linker_heap)
              return linker_heap_scores, linker_heap
            def apply_bottleneck(heap_scores, heap, bottleneck):
              ″″″
              Cluster sequences and output top-scoring sequence per cluster.
              ″″″
              # Can just pick best output
              if bottleneck == 1:
                return heap[np.argmax(heap_scores)]
              # Calculate features for each linker sequence i.e. edit distance to all other linker
            sequences
              features = np.zeros((len(heap), len(heap)), dtype=int)
              for i, seq_x in enumerate(heap):
                for j, seq_y in enumerate(heap):
                  features[i, j] = levenshtein_distance(seq_x, seq_y)
              # Cluster linker sequences
              clusters = HAC(n_clusters=bottleneck, linkage=“complete”).fit_predict(features)
              # Output highest-scoring linker sequence from each cluster
              output = [ ]
              heap_scores = np.array(heap_scores)
              for cluster_num in range(bottleneck):
                cluster_scores = clusters == cluster_num
                for subscore_num in range(heap_scores.shape[1]):
                  subscore_maxed = (heap_scores[:, subscore_num]
                            == np.max(heap_scores[cluster_scores, subscore_num]))
                  cluster_scores = np.logical_and(cluster_scores, subscore_maxed)
                idx_maxed = np.where(cluster_scores)[0]
                output.append(heap[np.random.choice(idx_maxed)])
              return output
            def pegLIT(seq_spacer, seq_scaffold, seq_template, seq_pbs, seq_motif,
                  linker_pattern=“NNNNNNNN”, ac_thresh=0.5, u_thresh=3, n_thresh=3,
            topn=100,
                  epsilon=1e-2, num_repeats=10, num_steps=250, temp_init=0.15,
            temp_decay=0.95,
                  bottleneck=1):
              ″″″
              Optimizes+bottlenecks linker for an inputted pegRNA. Outputs linker
            recommendation(s).
              ″″″
              # Simulated annealing to optimize linker sequence
              linker_heap_scores, linker_heap = optimize(
                seq_spacer, seq_scaffold, seq_template, seq_pbs, seq_motif,
                linker_pattern=linker_pattern, ac_thresh=ac_thresh, u_thresh=u_thresh,
                n_thresh=n_thresh, topn=topn, epsilon=epsilon, num_repeats=num_repeats,
                num_steps=num_steps, temp_init=temp_init, temp_decay=temp_decay)
              # Sample diverse sequences
              linker_output = apply_bottleneck(linker_heap_scores, linker_heap,
            bottleneck=bottleneck)
              return linker_output
            # Example usage for HEK3 +1 FLAG ins
            print(pegLIT(
              seq_spacer=“GGCCCAGACTGAGCACGTGA”,
            seq_scaffold=“GTTTTAGAGCTAGAAATAGCAAGTTAAAATAAGGCTAGTCC
            GTTAT”
                    “CAACTTGAAAAAGTGGCACCGAGTCGGTGC”,
            seq_template=“TGGAGGAAGCAGGGCTTCCTTTCCTCTGCCATCACTTATCG”
                    “TCGTCATCCTTGTAATC”,
              seq_pbs=“CGTGCTCAGTCTG”,
              seq_motif=“CGCGGTTCTATCTAGTTACGCGTTAAACCAACTAGAA”))
Sequences shown are: seq_spacer (SEQ ID NO: 490); seq_scaffold (top) (SEQ ID NO: 491);
seq_scaffold (bottom) (SEQ ID NO: 492); seq_template (top) (SEQ ID NO: 493); seq_template
(bottom) (SEQ ID NO: 494); seq_pbs (SEQ ID NO: 495); seq_motif (SEQ ID NO: 219).
```

### Example 5: Additional Strategies for Improving Prime Editing

Additional strategies for improving prime editing were also developed. These include three broad areas in which prime editing can be improved, as shown in FIG. 131: 1) recognition of the target nucleic acid; 2) installation of the edit(s); and 3) resolution of the edited DNA heteroduplex. The following example focuses on increasing editing efficiency by improving recognition of the target nucleic acid, specifically through reducing interactions between the PBS and spacer sequences on the pegRNA. Relative to sgRNAs, pegRNAs and epegRNAs may sometimes have reduced target site engagement and reduced binding to Cas9. PBS:spacer interactions can limit prime editing efficiency by reducing Cas9 affinity (FIG. 132). Such interactions are also necessary, however, in order for PBS:protospacer binding to occur. As shown in FIG. 132, a shorter PBS can result in improved binding affinity to Cas9. Strategies for reducing PBS:spacer interactions were therefore explored, including 1) occlusion of the PBS with toeholds that dissociate upon Cas9 binding; 2) delivery of the pegRNA template in trans via the nicking sgRNA; and 3) introducing chemical and/or genetic modifications that differentially affect PBS:spacer and PBS:protospacer interactions.

First, the strategy of occluding the PBS with toeholds that dissociate upon Cas9 binding was explored. It was observed that toeholds can inhibit both PBS:spacer and PBS:protospacer interactions if independent of Cas9 binding (FIG. 133). The MS2 hairpin was fused to the 3′ end of the pegRNA, while the MS2 bacteriophage coat protein was fused to the reverse transcriptase of the prime editor. As shown in FIG. 134, the toehold can be competed off by PE2 binding due to competitive RNA-protein interactions. Several design considerations should be taken account when using this strategy, including 1) the interdependence of the lengths of both the Cas9-RT and the RT-MS2 linkers, the pegRNA extension and PBS linkers, the toehold linker, and the linker between the MS2 aptamer and toehold; 2) the toehold length dependence upon PBS melting temperature and site accessibility; 3) optimization for each site; and 4) tolerance for a non-interacting 17 nucleotide PBS. Both N-terminal and C-terminal fusions of MS2 to PE2 were tested. It was discovered that use of C-terminal MS2 fusions results in superior editing efficiency to N-terminal fusions at HEK3 (FIG. 135). It was observed that MS2 tagging of PE2 provides benefits in editing efficiency compared to untagged PE2 using various pegRNAs (FIG. 136). PE2-MS2 fusions comprising either an xten-16aa linker or an xten-33aa linker were both tested relative to PE2-xten without an MS2 fusion. It was also observed that MS2 and toeloop tagging rescues long primer binding sites (FIG. 137). Overall, the strategy of occluding the PBS with toeholds that dissociate upon Cas9 binding shows some benefits in editing efficiency at various genomic sites, and particularly ones that are normally edited at lower efficiency due to low PBS:protospacer stability. It was also shown that epegRNAs motifs can be bi-functional, improving pegRNA stability.

Next, the strategy of delivering the pegRNA template in trans via the nicking sgRNA was explored. It was found hypothesized that the pegRNA extension can could be moved onto the nicking guide to completely avoid PBS-spacer interactions (FIG. 138). Several design considerations should also be taken into account when using this strategy, including: 1) the impact of an extended template as a linker on flap resolution; 2) optimization of the nicking spacer; and 3) the need for both PE complexes to be present on the genome simultaneously. It was observed that this strategy enables prime editing at DMNT1, HEK3, PRNP, RUNX1, and VEGFA (FIG. 139).

FIG. 140 shows a model based on mismatch identity and position within the PBS relative to the nick.

FIG. 141 shows that mutations to the PBS are tolerated or in some circumstances enhance PE activity and fit an initial model where mutation location and identity determine PE efficiency.

FIG. 142 shows that longer PBS (RNF2, 15 nt) do not tolerate mutations, potentially because they inhibit PBS:protospacer interactions excessively.

FIG. 143 shows that mutations to the PBS can improve PE efficiency for pegRNAs with shorter optimal PBS's. MutPBS epegRNAs have a mutPBS of 17 with 4 consecutive mutations (HEK3, DNMT1, PRNP) or a mutPBS of fifteen with four consecutive mutations (RNF2), followed by an 8 nt linker and tevopreQ₁.

FIG. 144 shows that mutPBS improvements can provide additional enhancements in editing efficiency when used in combination with epegRNAs.

FIG. 145 provides a schematic of twin prime editing. Twin prime editing is particularly useful for making large edits because the flaps are exogenous and can only base pair to one another.

FIG. 146 shows nicking of the intervening region in twin prime editing to reduce competitive homology for improved editing efficiency. The extra nick (or multiple nicks) degrades the region of the genome between the two flaps, reducing the complexity of intermediates and improving yield.

FIG. 147 shows MECP2 twin prime editing with an accessory nick.

### Example 6: Treating CDKL5 Deficiency Disorder by Prime Editing

CDKL5 deficiency disorder is a genetic disease characterized by seizures that begin early in life after birth, with subsequent delays in many aspects of development. The seizures associated with CDKL5 deficiency disorder are often found to change in character with age. The most common type of seizure experienced by affected individuals is called generalized tonic-clonic seizure (aka Grand-mal seizure), which involves a loss of consciousness, muscle rigidity, and bodily convulsions. Tonic seizures represent another major type of seizure affiliated with CDKL5 deficiency disorder and can be characterized by abnormal muscle contractions. Another common seizure type is an epileptic spasm, which involves short episodes of involuntary muscle jerks. Seizures occur daily in most people with CDKL5 deficiency disorder, although seizure-free periods can be experienced. Seizures in CDKL5 deficiency disorder are typically resistant to treatment.

CDKL5 deficiency disorder is also associated with impaired development in children. Such children have severe intellectual disability with significantly limited speech. In addition, the development of gross motor skills (e.g., walking, sitting, and standing) is delayed or completely absent in certain individuals. In fact, only about one-third of affected individuals are able to walk unassisted. Fine motor skills are also impaired, with only about half of affected individuals having meaningful use of their hands. Vision, too, is impaired in many individuals affected by CDKL5 deficiency disorder.

CDKL5 deficiency disorder is caused by mutations in the CDKL5 gene. This gene provides instructions for making a protein that is essential for normal brain development and function. In particular, mutations in the CDKL5 gene reduce the amount of functional CDKL5 protein or alter its activity in neurons. A shortage (deficiency) of CDKL5 or impairment of its function disrupts brain development, but it is unclear how these changes cause the specific features of CDKL5 deficiency disorder.

Current treatments for CDKL5 mutations/deficiencies are primarily focused on managing symptoms. However, there are currently no treatments that improve the neurological outcome of subjects with CDKL5 mutations or deficiencies or that can correct the mutations in the CDKL5 gene that cause the disorder. As such, there exists a need for gene therapy approaches for treating CDKL5 deficiency disorder.

In the present disclosure, a prime editor (e.g., PE2) was used in complex with a pegRNA as shown in FIG. 148 to correct multiple pathogenic mutations in the CDKL5 gene simultaneously (including correcting the V172I, A173D, R175S, W176G, W176R, Y177C, R178P, P180L, E181A, and L182P mutations). A single prime editor (e.g., PE2) complexed with a single pegRNA was also shown to be capable of correcting a multitude of pathogenic mutations at positions +4, +8, +12, +17, +21, and +25 relative to position 1 of the PAM sequence (i.e., the nucleotide in the 5′-most position; FIG. 149).

### Example 7: Method of Correcting Multiple Mutations in CDKL5 in Mice Using a Single Guide RNA

The following Example describes the optimization of installation of the pathogenic 1412delA mutation in mouse cells. N2A cells were used for this work as these cells are derived from a neuroblastoma, and CDKL5 Deficiency Disorder (CDD) is largely a neurological disease. Extensive efforts were undertaken to optimize the pegRNA and nicking guides and install this mutation. One such example of this optimization using DNA plasmid transfection is provided in FIG. 150. The PE system, nicking guides, and pegRNA parameters are detailed on the X-axis. The “13_20” pegRNA was used for subsequent synthetic pegRNAs for electroporations.

N2A cells were next electroporated with either in vitro transcribed PE mRNA, synthetic epegRNA, and synthetic guide RNA (PE3), or the aforementioned substrates with mMLH1neg mRNA (PE5) (FIG. 151). The nicking guide location (NG1 vs NG3) was also varied. It was concluded that the PE5 system with NG1 gives the highest installation percentage, and PE5 with NG3 gives the best desired edit to indel ratio.

A similar experiment was then performed with the addition of a seed edit being encoded by the epegRNAs, as well as the desired 1412delA mutation (FIG. 152). The seed edit is silent, and therefore not anticipated to be pathogenic since the amino acid sequence is not changed. Two standard nicking guides were used for PE5 (NG1, NG3), in addition to testing a nicking guide for the PE5b strategy. The length of the reverse transcriptase template differs between pegRNA 081 and 082. It was concluded that pegRNA 081 was the most efficient, installing the desired mutation at an efficiency of about 70%.

Because CDKL5 is caused by dominant mutations on the X chromosome, female patients usually have one healthy allele. The effect of indels at this healthy allele is unknown, and it could be targeted with the canonical PE (SpCas9 PE). New pegRNAs were designed (Table E7) that require an SpCas9-NRCH PE and 1) would not target the healthy allele, and 2) would not be good substrates for subsequent editing once the first editing event has happened because the PAM would be disrupted.

Finally, one pegRNA was used to correct multiple pathogenic alleles. Since mutations are de novo, it is extremely rare for any two patients to have the same mutation. However, there are loci in the CDKL5 gene that are more likely to harbor these pathogenic mutations than others. One such locus is Exon 8. Multiple pathogenic CDKL5 alleles were installed in HEK293T cells via plasmid transfection (FIG. 153). The two pegRNA parameters are described on the X-axis.

### Example 8: Prime Editing of the CDKL5 Locus with PE4 and PE5

PE4max and PE5max (FIGS. 154 and 155) were applied to introduce a silent C•G-to-T•A mutation at a CDKL5 site known to contain a causative mutation for CDKL5 deficiency disorder, a severe neurodevelopmental condition (Olson et al., 2019). It was observed that PE4max increased average prime editing efficiency over PE2 by 29-fold in HeLa cells and 2.1-fold in HEK293T cells (FIG. 156). Notably, PE4max editing efficiencies (8.6% editing with 0.19% indels in HeLa cells, and 20% editing with 0.26% indels in HEK293T cells) were similar to or exceeded those of PE3 (4.5% editing with 1.5% indels in HeLa cells, and 24% editing with 5.4% indels in HEK293T cells), but with far fewer indels. In addition, PE5max improved disease-relevant allele conversion over PE3 by an average of 6.1-fold in HeLa cells and 1.5-fold in HEK293T cells, and enhanced edit:indel purity by 6.4-fold in HeLa cells and by 3.5-fold in HEK293T cells (FIG. 156).

Next, PE4 and PE5 editing systems were evaluated in a cell model of genetic disease and in primary human cells. The pathogenic CDKL5 c.1412delA mutation in human induced pluripotent stem cells (iPSCs) derived from a patient heterozygous for this allele was corrected (Chen et al., 2021). Electroporation of these iPSCs with PE3 components (in vitro-transcribed PE2 mRNA and synthetic pegRNAs and nicking sgRNAs) yielded 17% correction of editable pathogenic alleles and 20% total indel products (FIGS. 157 and 158). Co-electroporation of these components with MLH1dn mRNA for PE5 editing elevated correction efficiency to 34% and lowered the frequency of indels to 6.1%. To further minimize indels, PE4 and PE5b strategies were also used. Without complementary-strand nicking, it was observed that MLH1dn improved allele correction from 4.0% (PE2) to 10% (PE4) with few indels (<0.34%) (FIGS. 157 and 158). Similarly, PE3b resulted in 13% editing of the mutant allele with 4.8% indels, while PE5b elevated editing to 27% with 3.8% indels. Across the PE4, PE5, and PE5b systems tested, MLH1dn enhances correction of the pathogenic CDKL5 c.1412delA mutation by 2.2-fold in efficiency and 3.6-fold in outcome purity in patient-derived iPSCs.

MLH1dn and epegRNAs were also combined for CDKL5 editing (FIG. 159). Editing efficiency of a CDKL5 c.1412 A to G mutation in HEK293T cells was increased through the use of MLH1dn (PE4 and PE5) and epegRNAs. Finally, the nicking sgRNA was also optimized for prime editing at CDKL5 (FIG. 160). The editing efficiency of installation of a CDKL5 silent +1 C to T mutation (c.1412delA site) in HEK293T cells was increased through such optimization. Sequences of the guide RNAs used in this Example are provided in Table E8.

### Example 9: PAM Variant Prime Editors for Editing the CDKL5 Locus

A high level of insertion and deletion byproducts (indels) in addition to the intended prime edit was observed when PE4 and PE5 were used to correct the CDKL5 c.1412delA mutation in heterozygous human patient-derived induced pluripotent stem cells. It was hypothesized that many of these indels are caused by attempted prime editing of the wild-type allele, which does not contain the c.1412delA mutation. Specifically, because the c.1412delA mutation is far from the targeted protospacer for SpCas9-PE, this protospacer will still be nicked even for the wild-type allele, thereby generating indel byproducts (FIG. 161).

To mitigate indels, prime editors were developed that target and nick the DNA only in the presence of the c.1412delA mutation. Prime editors were thus generated that use NRCH and NRTH SpCas9 variants (as described in International Patent Application Publication No. WO 2020/041751). NRCH SpCas9-PE and NRTH SpCas9-PE can target the c.1412delA mutation specifically, such that they are unable bind and nick the wild-type CDKL5 allele (FIGS. 162 and 163).

Thus, NRCH SpCas9-PE and NRTH SpCas9-PE will only correct the CDKL5 c.1412delA mutation if the mutation is present, which should minimize indel byproducts. The pegRNA and nicking sgRNA sequences used in this strategy are provided in Table E9.

### Example 10: Prime Editors for Installation of CDKL5 Mutations

Prime editing guide RNAs (pegRNAs) with varying primer binding site (PBS) and template lengths were screened to identify those that enabled the most efficient installation of a transition point mutation at c.1412 in the CDKL5 gene in HEK293T cells using the PE2 prime editor (FIG. 164). Next, the choice of nicking guide used in the PE3 prime editor system was optimized, further improving the efficiency of editing at c.1412 (FIG. 165). A coding-silent transition was also incorporated within the seed region of the protospacer targeted by the pegRNA to further improve editing efficiency (FIG. 165). The guide RNA sequences used in this strategy are provided in Table E10. Overall, the pegRNA CDKL5h37 and the epegRNA JNpeg0953 showed the highest editing efficiency.

### SEQUENCES

The following sequences in Tables E1-E6 are referred to throughout Example 4 and in the associated Drawings.

Lengthy table referenced here
US20230357766A1-20231109-T00001
Please refer to the end of the specification for access instructions.

Lengthy table referenced here
US20230357766A1-20231109-T00002
Please refer to the end of the specification for access instructions.

Lengthy table referenced here
US20230357766A1-20231109-T00003
Please refer to the end of the specification for access instructions.

Lengthy table referenced here
US20230357766A1-20231109-T00004
Please refer to the end of the specification for access instructions.

Lengthy table referenced here
US20230357766A1-20231109-T00005
Please refer to the end of the specification for access instructions.

Lengthy table referenced here
US20230357766A1-20231109-T00006
Please refer to the end of the specification for access instructions.

Lengthy table referenced here
US20230357766A1-20231109-T00007
Please refer to the end of the specification for access instructions.

Lengthy table referenced here
US20230357766A1-20231109-T00008
Please refer to the end of the specification for access instructions.

Lengthy table referenced here
US20230357766A1-20231109-T00009
Please refer to the end of the specification for access instructions.

Lengthy table referenced here
US20230357766A1-20231109-T00010
Please refer to the end of the specification for access instructions.

### REFERENCES FOR EXAMPLE 4

1. Komor, A. C., Badran, A. H. & Liu, D. R. CRISPR-Based Technologies for the Manipulation of Eukaryotic Genomes. Cell 168, 20-36 (2017).
2. Anzalone, A. V., Koblan, L. W. & Liu, D. R. Genome editing with CRISPR-Cas nucleases, base editors, transposases and prime editors. Nat Biotechnol 38, 824-844 (2020).
3. Cullot, G. et al. CRISPR-Cas9 genome editing induces megabase-scale chromosomal truncations. Nat Commun 10, 1136 (2019).
4. Kosicki, M., Tomberg, K. & Bradley, A. Repair of double-strand breaks induced by CRISPR-Cas9 leads to large deletions and complex rearrangements. Nat Biotechnol 36, 765-771 (2018).
5. Boroviak, K., Fu, B., Yang, F., Doe, B. & Bradley, A. Revealing hidden complexities of genomic rearrangements generated with Cas9. Sci Rep 7, 12867 (2017).
6. Enache, O. M. et al. Cas9 activates the p53 pathway and selects for p53-inactivating mutations. Nat Genet 52, 662-668 (2020).
7. Haapaniemi, E., Botla, S., Persson, J., Schmierer, B. & Taipale, J. CRISPR-Cas9 genome editing induces a p53-mediated DNA damage response. Nat Med 24, 927-930 (2018).
8. Ihry, R. J. et al. p53 inhibits CRISPR-Cas9 engineering in human pluripotent stem cells. Nat Med 24, 939-946 (2018).
9. Leibowitz, M. L. et al. Chromothripsis as an on-target consequence of CRISPR-Cas9 genome editing. Preprint at https://www.biorxiv.org/content/10.1101/2020.07.13.200998v1 (2020).
10. Burgio, G. & Teboul, L. Anticipating and Identifying Collateral Damage in Genome Editing. Trends Genet 36, 905-914 (2020).
11. Cox, D. B., Platt, R. J. & Zhang, F. Therapeutic genome editing: prospects and challenges. Nat Med 21, 121-131 (2015).
12. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
13. Gaudelli, N. M. et al. Programmable base editing of A*T to G*C in genomic DNA without DNA cleavage. Nature 551, 464-471 (2017).
14. Anzalone, A. V. et al. Search-and-replace genome editing without double-strand breaks or donor DNA. Nature 576, 149-157 (2019).
15. Houseley, J. & Tollervey, D. The many pathways of RNA degradation. Cell 136,763-776 (2009).
16. Hendel, A. et al. Chemically modified guide RNAs enhance CRISPR-Cas genome editing in human primary cells. Nat Biotechnol 33, 985-989 (2015).
17. Geisberg, J. V., Moqtaderi, Z., Fan, X., Ozsolak, F. & Struhl, K. Global analysis of mRNA isoform half-lives reveals stabilizing and destabilizing elements in yeast. Cell 156, 812-824 (2014).
18. Wu, X. & Bartel, D. P. Widespread Influence of 3′-End Structures on Mammalian mRNA Processing and Stability. Cell 169, 905-917 e911 (2017).
19. Brown, J. A. et al. Structural insights into the stabilization of MALAT1 noncoding RNA by a bipartite triple helix. Nat Struct Mol Biol 21, 633-640 (2014).
20. MacFadden, A. et al. Mechanism and structural diversity of exoribonuclease-resistant RNA structures in flaviviral RNAs. Nat Commun 9, 119 (2018).
21. Pijlman, G. P. et al. A highly structured, nuclease-resistant, noncoding RNA produced by flaviviruses is required for pathogenicity. Cell Host Microbe 4, 579-591 (2008).
22. Roth, A. et al. A riboswitch selective for the queuosine precursor preQ1 contains an unusually small aptamer domain. Nat Struct Mol Biol 14, 308-317 (2007).
23. Anzalone, A. V., Lin, A. J., Zairis, S., Rabadan, R. & Cornish, V. W. Reprogramming eukaryotic translation with ligand-responsive synthetic RNA switches. Nat Methods 13, 453-458 (2016).
24. Houck-Loomis, B. et al. An equilibrium-dependent retroviral mRNA switch regulates translational recoding. Nature 480, 561-564 (2011).
25. Lorenz, R. et al. ViennaRNA package 2.0. Algorithms Mol Biol 6, 26 (2011).
26. Schene, I. F. et al. Prime editing for functional repair in patient-derived disease models. Nat Commun 11, 5352 (2020).
27. Kim, D. Y., Moon, S. B., Ko, J. H., Kim, Y. S. & Kim, D. Unbiased investigation of specificities of prime editing systems in human cells. Nucleic Acids Res 48, 10576-10589 (2020).
28. Gao, P. et al. Prime editing in mice reveals the essentiality of a single base in driving tissue specific gene expression. Preprint at www.biorxiv.org/content/10.1101/2020.11.07.372748v3.full.pdf (2020).
29. Tsai, S. Q. et al. GUIDE-seq enables genome-wide profiling of off-target cleavage by CRISPR-Cas nucleases. Nat Biotechnol 33, 187-197 (2015).
30. Ibrahim, H., Wilusz, J. & Wilusz, C. J. RNA recognition by 3′-to-5′ exonucleases: the substrate perspective. Biochim biophys acta 1779, 256-265 (2008).
31. Green, L., Kim, C. H., Bustamante, C. & Tinoco, I., Jr. Characterization of the mechanical unfolding of RNA pseudoknots. J Mol Biol 375, 511-528 (2008).
32. Chavez, A. et al. Highly efficient Cas9-mediated transcriptional programming. Nat Methods 12, 326-328 (2015).
33. Hu, J. H. et al. Evolved Cas9 variants with broad PAM compatibility and high DNA specificity. Nature 556, 57-63 (2018).
34. Nahar, S. et al. A G-quadruplex motif at the 3′ end of sgRNAs improves CRISPR-Cas9 based genome editing efficiency. Chem Commun 54, 2377-2380 (2018).
35. Steckelberg, A. L. et al. A folded viral noncoding RNA blocks host cell exoribonucleases through a conformationally dynamic RNA structure. Proc Natl Acad Sci USA 115, 6404-6409 (2018).
36. Cate, J. H. et al. Crystal structure of a group I ribozyme domain: principles of RNA packing. Science 273, 1678-1685 (1996).
37. Fedor, M. J. & Westhof, E. Ribozymes: the first 20 years. Mol Cell 10, 703-704 (2002).
38. Chen, X., Zaro, J. L. & Shen, W. C. Fusion protein linkers: property, design and functionality. Adv Drug Deliv Rev 65, 1357-1369 (2013).
39. Win, M. N. & Smolke, C. D. A modular and extensible RNA-based gene-regulatory platform for engineering cellular function. Proc Natl Acad Sci USA 104, 14283-14288 (2007).
40. Nielsen, S., Yuzenkova, Y. & Zenkin, N. Mechanism of eukaryotic RNA polymerase III transcription termination. Science 340, 1577-1580 (2013).
41. Huang, T. P., Newby, G. A. & Liu, D. R. Precision genome editing using cytosine and adenine base editors in mammalian cells. Nat Protoc (2021).
42. Basila, M., Kelley, M. L. & Smith, A. V. B. Minimal 2′-O-methyl phosphorothioate linkage modification pattern of synthetic guide RNAs for increased stability and efficient CRISPR-Cas9 gene editing avoiding cellular toxicity. PLoS One 12, e0188593 (2017).
43. Mead, S. et al. A novel protective prion protein variant that colocalizes with Kuru exposure. N Engl J Med 361, 2056-2065 (2009).
44. Asante, E. A. et al. A naturally occurring variant of the human prion protein completely prevents prion disease. Nature 522, 478-481 (2015).
45. Kim, H. K. et al. Predicting the efficiency of prime editing guide RNAs in human cells. Nat Biotechnol (2020).
46. Jonsson, T. et al. A mutation in APP protects against Alzheimer's disease and age-related cognitive decline. Nature 488, 96-99 (2012).
47. Abifadel, M. et al. Mutations in PCSK9 cause autosomal dominant hypercholesterolemia. Nat Genet 34, 154-156 (2003).
48. Bustami, J. et al. Cholesteryl ester transfer protein (CETP) I405V polymorphism and cardiovascular disease in eastern European Caucasians—a cross-sectional study. BMC Geriatr 16, 144 (2016).
49. Flannick, J. et al. Loss-of-function mutations in SLC30A8 protect against type 2 diabetes. Nat Genet 46, 357-363 (2014).
50. Sakuntabhai, A. et al. A variant in the CD209 promoter is associated with severity of dengue disease. Nat Genet 37, 507-513 (2005).
51. Olson, H. E. et al. Cyclin-Dependent Kinase-Like 5 Deficiency Disorder: Clinical Review. Pediatr Neurol 97, 18-25 (2019).
52. Al-Saaidi, R. et al. The LMNA mutation p.Arg321Ter associated with dilated cardiomyopathy leads to reduced expression and a skewed ratio of lamin A and lamin C proteins. Exp Cell Res 319, 3010-3019 (2013).
53. Ip, J. P. K., Mellios, N. & Sur, M. Rett syndrome: insights into genetic, molecular and circuit mechanisms. Nat Rev Neurosci 19, 368-382 (2018).
54. Christodoulou, J., Grimm, A., Maher, T. & Bennetts, B. RettBASE: The IRSA MECP2 variation database—a new mutation database in evolution. Hum Mutat 21, 466-472 (2003).
55. Dwivedi, O. P. et al. Loss of ZnT8 function protects against diabetes by enhanced insulin secretion. Nat Genet 51, 1596-1606 (2019).
56. Thyme, S. B., Akhmetova, L., Montague, T. G., Valen, E. & Schier, A. F. Internal guide RNA interactions interfere with Cas9-mediated cleavage. Nat Commun 7, 11750 (2016).
57. Boyle, E. A. et al. Quantification of Cas9 binding and cleavage across diverse guide sequences maps landscapes of target engagement. Science Advances, in press (2021).
58. Gaudelli, N. M. et al. Directed evolution of adenine base editors with increased activity and therapeutic application. Nat Biotechnol 38, 892-900 (2020).
59. Clement, K. et al. CRISPResso2 provides accurate and rapid genome editing sequence analysis. Nat Biotechnol 37, 224-226 (2019).
60. Pandey, S., Agarwala, P. & Maiti, S. Effect of loops and G-quartets on the stability of RNA G-quadruplexes. J Phys Chem B 117, 6896-6905 (2013).
61. Engler, C., Gruetzner, R., Kandzia, R. & Marillonnet, S. Golden gate shuffling: a one-pot DNA shuffling method based on type IIs restriction enzymes. PLoS One 4, e5553 (2009).

### REFERENCES

All of the following references are each incorporated herein by reference in their entireties.
1. Jinek, M. et al. A Programmable Dual-RNA-Guided DNA Endonuclease in Adaptive Bacterial Immunity. Science 337, 816-821 (2012).
2. Cong, L. et al. Multiplex Genome Engineering Using CRISPR/Cas Systems. Science 339, 819-823 (2013).
3. Komor, A. C., Badran, A. H. & Liu, D. R. CRISPR-Based Technologies for the Manipulation of Eukaryotic Genomes. Cell 168, 20-36 (2017).
4. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
5. Nishida, K. et al. Targeted nucleotide editing using hybrid prokaryotic and vertebrate adaptive immune systems. Science 353, aaf8729 (2016).
6. Gaudelli, N. M. et al. Programmable base editing of A•T to G•C in genomic DNA without DNA cleavage. Nature 551, 464-471 (2017).
7. ClinVar, July 2019.
8. Dunbar, C. E. et al. Gene therapy comes of age. Science 359, eaan4672 (2018).
9. Cox, D. B. T., Platt, R. J. & Zhang, F. Therapeutic genome editing: prospects and challenges. Nat. Med. 21, 121-131 (2015).
10. Adli, M. The CRISPR tool kit for genome editing and beyond. Nat. Commun. 9, 1911 (2018).
11. Kleinstiver, B. P. et al. Engineered CRISPR-Cas9 nucleases with altered PAM specificities. Nature 523, 481-485 (2015).
12. Kleinstiver, B. P. et al. High-fidelity CRISPR-Cas9 nucleases with no detectable genome-wide off-target effects. Nature 529, 490-495 (2016).
13. Hu, J. H. et al. Evolved Cas9 variants with broad PAM compatibility and high DNA specificity. Nature 556, 57-63 (2018).
14. Nishimasu, H. et al. Engineered CRISPR-Cas9 nuclease with expanded targeting space. Science 361, 1259-1262 (2018).
15. Jasin, M. & Rothstein, R. Repair of strand breaks by homologous recombination. Cold Spring Harb. Perspect. Biol. 5, a012740 (2013).
16. Paquet, D. et al. Efficient introduction of specific homozygous and heterozygous mutations using CRISPR/Cas9. Nature 533, 125-129 (2016).
17. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
18. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
19. Ihry, R. J. et al. p53 inhibits CRISPR-Cas9 engineering in human pluripotent stem cells. Nat. Med. 24, 939-946 (2018).
20. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
21. Srivastava, M. et al. An Inhibitor of Nonhomologous End-Joining Abrogates Double-Strand Break Repair and Impedes Cancer Progression. Cell 151, 1474-1487 (2012).
22. Chu, V. T. et al. Increasing the efficiency of homology-directed repair for CRISPR-Cas9-induced precise gene editing in mammalian cells. Nat. Biotechnol. 33, 543-548 (2015).
23. Maruyama, T. et al. Increasing the efficiency of precise genome editing with CRISPR-Cas9 by inhibition of nonhomologous end joining. Nat. Biotechnol. 33, 538-542 (2015).
24. Kim, Y. B. et al. Increasing the genome-targeting scope and precision of base editing with engineered Cas9-cytidine deaminase fusions. Nat. Biotechnol. 35, 371-376 (2017).
25. Li, X. et al. Base editing with a Cpf1-cytidine deaminase fusion. Nat. Biotechnol. 36, 324-327 (2018).
26. Gehrke, J. M. et al. An APOBEC3A-Cas9 base editor with minimized bystander and off-target activities. Nat. Biotechnol. (2018). doi:10.1038/nbt.4199
27. Rees, H. A. & Liu, D. R. Base editing: precision chemistry on the genome and transcriptome of living cells. Nat. Rev. Genet. 1 (2018). doi:10.1038/s41576-018-0059-1.
28. Ostertag, E. M. & Kazazian Jr, H. H. Biology of Mammalian L1 Retrotransposons. Annu. Rev. Genet. 35, 501-538 (2001).
29. Zimmerly, S., Guo, H., Perlman, P. S. & Lambowltz, A. M. Group II intron mobility occurs by target DNA-primed reverse transcription. Cell 82, 545-554 (1995).
30. Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
31. Feng, Q., Moran, J. V., Kazazian, H. H. & Boeke, J. D. Human L1 retrotransposon encodes a conserved endonuclease required for retrotransposition. Cell 87, 905-916 (1996).
32. Jinek, M. et al. Structures of Cas9 Endonucleases Reveal RNA-Mediated Conformational Activation. Science 343, 1247997 (2014).
33. Jiang, F. et al. Structures of a CRISPR-Cas9 R-loop complex primed for DNA cleavage. Science aad8282 (2016). doi:10.1126/science.aad8282
34. Qi, L. S. et al. Repurposing CRISPR as an RNA-Guided Platform for Sequence-Specific Control of Gene Expression. Cell 152, 1173-1183 (2013).
35. Tang, W., Hu, J. H. & Liu, D. R. Aptazyme-embedded guide RNAs enable ligand-responsive genome editing and transcriptional activation. Nat. Commun. 8, 15939 (2017).
36. Shechner, D. M., Hacisuleyman, E., Younger, S. T. & Rinn, J. L. Multiplexable, locus-specific targeting of long RNAs with CRISPR-Display. Nat. Methods 12, 664-670 (2015).
37. Anders, C. & Jinek, M. Chapter One—In vitro Enzymology of Cas9. in Methods in Enzymology (eds. Doudna, J. A. & Sontheimer, E. J.) 546, 1-20 (Academic Press, 2014).
38. Briner, A. E. et al. Guide RNA Functional Modules Direct Cas9 Activity and Orthogonality. Mol. Cell 56, 333-339 (2014).
39. Nowak, C. M., Lawson, S., Zerez, M. & Bleris, L. Guide RNA engineering for versatile Cas9 functionality. Nucleic Acids Res. 44, 9555-9564 (2016).
40. Sternberg, S. H., Redding, S., Jinek, M., Greene, E. C. & Doudna, J. A. DNA interrogation by the CRISPR RNA-guided endonuclease Cas9. Nature 507, 62-67 (2014).
41. Mohr, S. et al. Thermostable group II intron reverse transcriptase fusion proteins and their use in cDNA synthesis and next-generation RNA sequencing. RNA 19, 958-970 (2013).
42. Stamos, J. L., Lentzsch, A. M. & Lambowitz, A. M. Structure of a Thermostable Group II Intron Reverse Transcriptase with Template-Primer and Its Functional and Evolutionary Implications. Mol. Cell 68, 926-939.e4 (2017).
43. Zhao, C. & Pyle, A. M. Crystal structures of a group II intron maturase reveal a missing link in spliceosome evolution. Nat. Struct. Mol. Biol. 23, 558-565 (2016).
44. Zhao, C., Liu, F. & Pyle, A. M. An ultraprocessive, accurate reverse transcriptase encoded by a metazoan group II intron. RNA 24, 183-195 (2018).
45. Ran, F. A. et al. Genome engineering using the CRISPR-Cas9 system. Nat. Protoc. 8, 2281-2308 (2013).
46. Liu, Y., Kao, H.-I. & Bambara, R. A. Flap endonuclease 1: a central component of DNA metabolism. Annu. Rev. Biochem. 73, 589-615 (2004).
47. Krokan, H. E. & Bjpris, M. Base Excision Repair. Cold Spring Harb. Perspect. Biol.
5, (2013).
48. Kelman, Z. PCNA: structure, functions and interactions. Oncogene 14, 629-640 (1997).
49. Choe, K. N. & Moldovan, G.-L. Forging Ahead through Darkness: PCNA, Still the Principal Conductor at the Replication Fork. Mol. Cell 65, 380-392 (2017).
50. Li, X., Li, J., Harrington, J., Lieber, M. R. & Burgers, P. M. Lagging strand DNA synthesis at the eukaryotic replication fork involves binding and stimulation of FEN-1 by proliferating cell nuclear antigen. J. Biol. Chem. 270, 22109-22112 (1995).
51. Tom, S., Henricksen, L. A. & Bambara, R. A. Mechanism whereby proliferating cell nuclear antigen stimulates flap endonuclease 1. J. Biol. Chem. 275, 10498-10505 (2000).
52. Tanenbaum, M. E., Gilbert, L. A., Qi, L. S., Weissman, J. S. & Vale, R. D. A protein-tagging system for signal amplification in gene expression and fluorescence imaging. Cell 159, 635-646 (2014).
53. Bertrand, E. et al. Localization of ASH1 mRNA particles in living yeast. Mol. Cell 2, 437-445 (1998).
54. Dahlman, J. E. et al. Orthogonal gene knockout and activation with a catalytically active Cas9 nuclease. Nat. Biotechnol. 33, 1159-1161 (2015).
55. Tsai, S. Q. et al. GUIDE-seq enables genome-wide profiling of off-target cleavage by CRISPR-Cas nucleases. Nat. Biotechnol. 33, 187-197 (2015).
56. Tsai, S. Q. et al. CIRCLE-seq: a highly sensitive in vitro screen for genome-wide CRISPR-Cas9 nuclease off-targets. Nat. Methods 14, 607-614 (2017).
57. Schek N, Cooke C, Alwine J C. Molecular and Cellular Biology. (1992).
58. Gil A, Proudfoot NJ. Cell. (1987).
59. Zhao, B. S., Roundtree, I. A., He, C. Nat Rev Mol Cell Biol. (2017).
60. Rubio, M. A. T., Hopper, A. K. Wiley Interdiscip Rev RNA (2011).
61. Shechner, D. M., Hacisuleyman E., Younger, S. T., Rinn, J. L. Nat Methods. (2015).
62. Paige, J. S., Wu, K. Y., Jaffrey, S. R. Science (2011).
63. Ray D., . . . Hughes T R. Nature (2013).
64. Chadalavada, D. M., Cerrone-Szakal, A. L., Bevilacqua, P. C. RNA (2007).
65. Forster A C, Symons R H. Cell. (1987).
66. Weinberg Z, Kim P B, Chen T H, Li S, Harris K A, Lunse C E, Breaker R R. Nat. Chem. Biol. (2015).
67. Feldstein P A, Buzayan J M, Bruening G. Gene (1989).
68. Saville B J, Collins R A. Cell. (1990).
69. Winkler W C, Nahvi A, Roth A, Collins J A, Breaker R R. Nature (2004).
70. Roth A, Weinberg Z, Chen A G, Kim P G, Ames T D, Breaker R R. Nat Chem Biol. (2013).
71. Choudhury R, Tsai Y S, Dominguez D, Wang Y, Wang Z. Nat Commun. (2012).
72. MacRae IJ, Doudna J A. Curr Opin Struct Biol. (2007).
73. Bernstein E, Caudy A A, Hammond S M, Hannon G J Nature (2001).
74. Filippov V, Solovyev V, Filippova M, Gill S S. Gene (2000).
75. Cadwell R C and Joyce G F. PCR Methods Appl. (1992).
76. McInerney P, Adams P, and Hadi M Z. Mol Biol Int. (2014).
77. Esvelt K M, Carlson J C, and Liu D R. Nature. (2011).
78. Naorem S S, Hin J, Wang S, Lee W R, Heng X, Miller J F, Guo H. Proc Natl Acad Sci USA (2017).
79. Martinez M A, Vartanian J P, Wain-Hobson S. Proc Natl Acad Sci USA (1994).
80. Meyer A J, Ellefson J W, Ellington A D. Curr Protoc Mol Biol. (2014).
81. Wang H H, Isaacs F J, Carr P A, Sun Z Z, Xu G, Forest C R, Church G M. Nature. (2009).
82. Nyerges A et al. Proc Natl Acad Sci USA. (2016).
83. Mascola J R, Haynes B F. Immunol Rev. (2013).
84. X. Wen, K. Wen, D. Cao, G. Li, R. W. Jones, J. Li, S. Szu, Y. Hoshino, L. Yuan, Inclusion of a universal tetanus toxoid CD4(+) T cell epitope P2 significantly enhanced the immunogenicity of recombinant rotavirus ΔVP8* subunit parenteral vaccines. Vaccine 32, 4420-4427 (2014).
85. G. Ada, D. Isaacs, Carbohydrate-protein conjugate vaccines. Clin Microbiol Infect 9, 79-85 (2003).
86. E. Malito, B. Bursulaya, C. Chen, P. L. Surdo, M. Picchianti, E. Balducci, M. Biancucci, A. Brock, F. Berti, M. J. Bottomley, M. Nissum, P. Costantino, R. Rappuoli, G. Spraggon, Structural basis for lack of toxicity of the diphtheria toxin mutant CRM197. Proceedings of the National Academy of Sciences 109, 5229 (2012).
87. J. de Wit, M. E. Emmelot, M. C. M. Poelen, J. Lanfermeijer, W. G. H. Han, C. van Els, P. Kaaijk, The Human CD4(+) T Cell Response against Mumps Virus Targets a Broadly Recognized Nucleoprotein Epitope. J Virol 93, (2019).
88. M. May, C. A. Rieder, R. J. Rowe, Emergent lineages of mumps virus suggest the need for a polyvalent vaccine. Int J Infect Dis 66, 1-4 (2018).
89. M. Ramamurthy, P. Rajendiran, N. Saravanan, S. Sankar, S. Gopalan, B. Nandagopal, Identification of immunogenic B-cell epitope peptides of rubella virus E1 glycoprotein towards development of highly specific immunoassays and/or vaccine. Conference Abstract, (2019).
90. U. S. F. Tambunan, F. R. P. Sipahutar, A. A. Parikesit, D. Kerami, Vaccine Design for H5N1 Based on B- and T-cell Epitope Predictions. Bioinform Biol Insights 10, 27-35 (2016).
91. Asante, E A. et. al. “A naturally occurring variant of the human prion protein completely prevents prion disease”. Nature. (2015).
92. Crabtree, G. R. & Schreiber, S. L. Three-part inventions: intracellular signaling and induced proximity. Trends Biochem. Sci. 21, 418-22 (1996).
93. Liu, J. et al. Calcineurin Is a Common Target of A and FKBP-FK506 Complexes. Cell 66, 807-815 (1991).
94. Keith, C. T. et al. A mammalian protein targeted by G1-arresting rapamycin-receptor complex. Nature 369, 756-758 (2003).
95. Spencer, D. M., Wandless, T. J., Schreiber, S. L. S. & Crabtree, G. R. Controlling signal transduction with synthetic ligands. Science 262, 1019-24 (1993).
96. Pruschy, M. N. et al. Mechanistic studies of a signaling pathway activated by the organic dimerizer FK1012. Chem. Biol. 1, 163-172 (1994).
97. Spencer, D. M. et al. Functional analysis of Fas signaling in vivo using synthetic inducers of dimerization. Curr. Biol. 6, 839-847 (1996).
98. Belshaw, P. J., Spencer, D. M., Crabtree, G. R. & Schreiber, S. L. Controlling programmed cell death with a cyclophilin-cyclosporin-based chemical inducer of dimerization. Chem. Biol. 3, 731-738 (1996).
99. Yang, J. X., Symes, K., Mercola, M. & Schreiber, S. L. Small-molecule control of insulin and PDGF receptor signaling and the role of membrane attachment. Curr. Biol. 8, 11-18 (1998).
100. Belshaw, P. J., Ho, S. N., Crabtree, G. R. & Schreiber, S. L. Controlling protein association and subcellular localization with a synthetic ligand that induces heterodimerization of proteins. Proc. Natl. Acad. Sci. 93, 4604-4607 (2002).
101. Stockwell, B. R. & Schreiber, S. L. Probing the role of homomeric and heteromeric receptor interactions in TGF-β signaling using small molecule dimerizers. Curr. Biol. 8, 761-773 (2004).
102. Spencer, D. M., Graef, I., Austin, D. J., Schreiber, S. L. & Crabtree, G. R. A general strategy for producing conditional alleles of Src-like tyrosine kinases. Proc. Natl. Acad. Sci. 92, 9805-9809 (2006).
103. Holsinger, L. J., Spencer, D. M., Austin, D. J., Schreiber, S. L. & Crabtree, G. R. Signal transduction in T lymphocytes using a conditional allele of Sos. Proc. Natl. Acad. Sci. 92, 9810-9814 (2006).
104. Myers, M. G. Insulin Signal Transduction and the IRS Proteins. Annu. Rev. Pharmacol. Toxicol. 36, 615-658 (1996).
105. Watowich, S. S. The erythropoietin receptor: Molecular structure and hematopoietic signaling pathways. J. Investig. Med. 59, 1067-1072 (2011).
106. Blau, C. A., Peterson, K. R., Drachman, J. G. & Spencer, D. M. A proliferation switch for genetically modified cells. Proc. Natd. Acad. Sci. 94, 3076-3081 (2002).
107. Clackson, T. et al. Redesigning an FKBP-ligand interface to generate chemical dimerizers with novel specificity. Proc. Natd. Acad. Sci. 95, 10437-10442 (1998).
108. Diver, S. T. & Schreiber, S. L. Single-step synthesis of cell-permeable protein dimerizers that activate signal transduction and gene expression. J. Am. Chem. Soc. 119,5106-5109(1997).
109. Guo, Z. F., Zhang, R. & Liang, F. Sen. Facile functionalization of FK506 for biological studies by the thiol-ene ‘click’ reaction. RSC Adv. 4, 11400-11403 (2014).
110. Robinson, D. R., Wu, Y.-M. & Lin, S.-F. The protein tyrosine kinase family of the human genome. Oncogene 19, 5548-5557 (2000).
111. Landrum, M. J. et al. ClinVar: public archive of interpretations of clinically relevant variants. Nucleic Acids Res. 44, D862-D868 (2016).
112. Jinek, M. et al. A Programmable Dual-RNA-Guided DNA Endonuclease in Adaptive Bacterial Immunity. Science 337, 816-821 (2012).
113. Cong, L. et al. Multiplex Genome Engineering Using CRISPR/Cas Systems. Science 339, 819-823 (2013).
114. Mali, P. et al. RNA-Guided Human Genome Engineering via Cas9. Science 339, 823-826 (2013).
115. Yang, H. et al. One-Step Generation of Mice Carrying Reporter and Conditional Alleles by CRISPR/Cas-Mediated Genome Engineering. Cell 154, 1370-1379 (2013).
116. Kim, S., Kim, D., Cho, S. W., Kim, J. & Kim, J.-S. Highly efficient RNA-guided genome editing in human cells via delivery of purified Cas9 ribonucleoproteins. Genome Res. 24, 1012-1019 (2014).
117. Orlando, S. J. et al. Zinc-finger nuclease-driven targeted integration into mammalian genomes using donors with limited chromosomal homology. Nucleic Acids Res. 38, e152-e152 (2010).
118. Tsai, S. Q. et al. GUIDE-seq enables genome-wide profiling of off-target cleavage by CRISPR-Cas nucleases. Nat. Biotechnol. 33, 187-197 (2015).
119. Suzuki, K. et al. In vivo genome editing via CRISPR/Cas9 mediated homology-independent targeted integration. Nature 540, 144-149 (2016).
120. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
121. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
122. Ihry, R. J. et al. p53 inhibits CRISPR-Cas9 engineering in human pluripotent stem cells. Nat. Med. 24, 939-946 (2018).
123. Chapman, J. R., Taylor, M. R. G. & Boulton, S. J. Playing the end game: DNA double-strand break repair pathway choice. Mol. Cell 47, 497-510 (2012).
124. Cox, D. B. T., Platt, R. J. & Zhang, F. Therapeutic genome editing: prospects and challenges. Nat. Med. 21, 121-131 (2015).
125. Paquet, D. et al. Efficient introduction of specific homozygous and heterozygous mutations using CRISPR/Cas9. Nature 533, 125-129 (2016).
126. Chu, V. T. et al. Increasing the efficiency of homology-directed repair for CRISPR-Cas9-induced precise gene editing in mammalian cells. Nat. Biotechnol. 33, 543-548 (2015).
127. Maruyama, T. et al. Increasing the efficiency of precise genome editing with CRISPR-Cas9 by inhibition of nonhomologous end joining. Nat. Biotechnol. 33, 538-542 (2015).
128. Rees, H. A., Yeh, W.-H. & Liu, D. R. Development of hRad51-Cas9 nickase fusions that mediate HDR without double-stranded breaks. Nat. Commun. 10, 1-12 (2019).
129. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
130. Gaudelli, N. M. et al. Programmable base editing of A•T to G•C in genomic DNA without DNA cleavage. Nature 551, 464-471 (2017).
131. Gao, X. et al. Treatment of autosomal dominant hearing loss by in vivo delivery of genome editing agents. Nature 553, 217-221 (2018).
132. Ingram, V. M. A specific chemical difference between the globins of normal human and sickle-cell anaemia haemoglobin. Nature 178, 792-794 (1956).
133. Myerowitz, R. & Costigan, F. C. The major defect in Ashkenazi Jews with Tay-Sachs disease is an insertion in the gene for the alpha-chain of beta-hexosaminidase. J. Biol. Chem. 263, 18587-18589 (1988).
134. Zielenski, J. Genotype and Phenotype in Cystic Fibrosis. Respiration 67, 117-133 (2000).
135. Mead, S. et al. A Novel Protective Prion Protein Variant that Colocalizes with Kuru Exposure. N. Engl. J. Med. 361, 2056-2065 (2009).
136. Marraffini, L. A. & Sontheimer, E. J. CRISPR interference limits horizontal gene transfer in staphylococci by targeting DNA. Science 322, 1843-1845 (2008).
137. Barrangou, R. et al. CRISPR provides acquired resistance against viruses in prokaryotes. Science 315, 1709-1712 (2007).
138. Jiang, F. & Doudna, J. A. CRISPR-Cas9 Structures and Mechanisms. Annu. Rev. Biophys. 46, 505-529 (2017).
139. Hille, F. et al. The Biology of CRISPR-Cas: Backward and Forward. Cell 172, 1239-1259 (2018).
140. Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
141. Liu, Y., Kao, H.-I. & Bambara, R. A. Flap endonuclease 1: a central component of DNA metabolism. Annu. Rev. Biochem. 73, 589-615 (2004).
142. Rees, H. A. & Liu, D. R. Base editing: precision chemistry on the genome and transcriptome of living cells. Nat. Rev. Genet. 19, 770 (2018).
143. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
144. Qi, L. S. et al. Repurposing CRISPR as an RNA-Guided Platform for Sequence-Specific Control of Gene Expression. Cell 152, 1173-1183 (2013).
145. Shechner, D. M., Hacisuleyman, E., Younger, S. T. & Rinn, J. L. Multiplexable, locus-specific targeting of long RNAs with CRISPR-Display. Nat. Methods 12, 664-670 (2015).
146. Tang, W., Hu, J. H. & Liu, D. R. Aptazyme-embedded guide RNAs enable ligand-responsive genome editing and transcriptional activation. Nat. Commun. 8, 15939 (2017).
147. Jinek, M. et al. Structures of Cas9 Endonucleases Reveal RNA-Mediated Conformational Activation. Science 343, 1247997 (2014).
148. Nishimasu, H. et al. Crystal Structure of Cas9 in Complex with Guide RNA and Target DNA. Cell 156, 935-949 (2014).
149. Jiang, F., Zhou, K., Ma, L., Gressel, S. & Doudna, J. A. A Cas9-guide RNA complex preorganized for target DNA recognition. Science 348, 1477-1481 (2015).
150. Baranauskas, A. et al. Generation and characterization of new highly thermostable and processive M-MuLV reverse transcriptase variants. Protein Eng. Des. Sel. 25, 657-668 (2012).
151. Gerard, G. F. et al. The role of template-primer in protection of reverse transcriptase from thermal inactivation. Nucleic Acids Res. 30, 3118-3129 (2002).
152. Arezi, B. & Hogrefe, H. Novel mutations in Moloney Murine Leukemia Virus reverse transcriptase increase thermostability through tighter binding to template-primer. Nucleic Acids Res. 37, 473-481 (2009).
153. Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res. 16, 265-277 (1988).
154. Shen, M. W. et al. Predictable and precise template-free CRISPR editing of pathogenic variants. Nature 563, 646-651 (2018).
155. Thuronyi, B. W. et al. Continuous evolution of base editors with expanded target compatibility and improved activity. Nat. Biotechnol. (2019). doi:10.1038/s41587-019-0193-0
156. Kim, Y. B. et al. Increasing the genome-targeting scope and precision of base editing with engineered Cas9-cytidine deaminase fusions. Nat. Biotechnol. 35, 371-376 (2017).
157. Koblan, L. W. et al. Improving cytidine and adenine base editors by expression optimization and ancestral reconstruction. Nat. Biotechnol. (2018). doi:10.1038/nbt.4172
158. Komor, A. C. et al. Improved base excision repair inhibition and bacteriophage Mu Gam protein yields C:G-to-T:A base editors with higher efficiency and product purity. Sci. Adv. 3, eaao4774 (2017).
159. Kleinstiver, B. P. et al. High-fidelity CRISPR-Cas9 nucleases with no detectable genome-wide off-target effects. Nature 529, 490-495 (2016).
160. Zuo, E. et al. Cytosine base editor generates substantial off-target single-nucleotide variants in mouse embryos. Science 364, 289-292 (2019).
161. Jin, S. et al. Cytosine, but not adenine, base editors induce genome-wide off-target mutations in rice. Science 364, 292-295 (2019).
162. Kim, D., Kim, D., Lee, G., Cho, S.-I. & Kim, J.-S. Genome-wide target specificity of CRISPR RNA-guided adenine base editors. Nat. Biotechnol. 37, 430-435 (2019).
163. Grunewald, J. et al. Transcriptome-wide off-target RNA editing induced by CRISPR-guided DNA base editors. Nature 569, 433-437 (2019).
164. Zhou, C. et al. Off-target RNA mutation induced by DNA base editing and its elimination by mutagenesis. Nature 571, 275-278 (2019).
165. Rees, H. A., Wilson, C., Doman, J. L. & Liu, D. R. Analysis and minimization of cellular RNA editing by DNA adenine base editors. Sci. Adv. 5, eaax5717 (2019).
166. Ostertag, E. M. & Kazazian Jr, H. H. Biology of Mammalian L1 Retrotransposons. Annu. Rev. Genet. 35, 501-538 (2001).
167. Griffiths, D. J. Endogenous retroviruses in the human genome sequence. Genome Biol. 2, REVIEWS1017 (2001).
168. Berkhout, B., Jebbink, M. & Zsiros, J. Identification of an Active Reverse Transcriptase Enzyme Encoded by a Human Endogenous HERV-K Retrovirus. J. Virol. 73, 2365-2375 (1999).
169. Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. J. Virol. 74, 10349-10358 (2000).
170. Dever, D. P. et al. CRISPR/Cas9 Beta-globin Gene Targeting in Human Hematopoietic Stem Cells. Nature 539, 384-389 (2016).
171. Park, S. H. et al. Highly efficient editing of the β-globin gene in patient-derived hematopoietic stem and progenitor cells to treat sickle cell disease. Nucleic Acids Res. doi:10.1093/nar/gkz475
172. Collinge, J. Prion diseases of humans and animals: their causes and molecular basis. Annu. Rev. Neurosci. 24, 519-550 (2001).
173. Asante, E. A. et al. A naturally occurring variant of the human prion protein completely prevents prion disease. Nature 522, 478-481 (2015).
174. Anzalone, A. V., Lin, A. J., Zairis, S., Rabadan, R. & Cornish, V. W. Reprogramming eukaryotic translation with ligand-responsive synthetic RNA switches. Nat. Methods 13, 453-458 (2016).
175. Badran, A. H. et al. Continuous evolution of Bacillus thuringiensis toxins overcomes insect resistance. Nature 533, 58-63 (2016).
176. Anders, C. & Jinek, M. Chapter One—In Vitro Enzymology of Cas9. in Methods in Enzymology (eds. Doudna, J. A. & Sontheimer, E. J.) 546, 1-20 (Academic Press, 2014).
177. Pirakitikulr, N., Ostrov, N., Peralta-Yahya, P. & Cornish, V. W. PCRless library mutagenesis via oligonucleotide recombination in yeast. Protein Sci. Publ. Protein Soc. 19, 2336-2346 (2010).
178. Clement, K. et al. CRISPResso2 provides accurate and rapid genome editing sequence analysis. Nat. Biotechnol. 37, 224-226 (2019).
179. Tsai, S. Q. et al. GUIDE-seq enables genome-wide profiling of off-target cleavage by CRISPR-Cas nucleases. Nat. Biotechnol. 33, 187-197 (2015).
180. Kleinstiver, B. P. et al. High-fidelity CRISPR-Cas9 nucleases with no detectable genome-wide off-target effects. Nature 529, 490-495 (2016).
181. Koblan, L. W. et al. Improving cytidine and adenine base editors by expression optimization and ancestral reconstruction. Nat. Biotechnol. (2018). doi:10.1038/nbt.4172
182. Baranauskas, A. et al. Generation and characterization of new highly thermostable and processive M-MuLV reverse transcriptase variants. Protein Eng. Des. Sel. 25, 657-668 (2012).
183. Schechner, D M, Hacisuleyman E., Younger S T, Rinn J L. sNat Methods 664-70 (2015).
184. Brown J A, et al. Nat Struct Mol Biol 633-40 (2014).
185. Conrad N A and Steitz J A. EMBO J 1831-41 (2005).
186. Bartlett J S, et al. Proc Natl Acad Sci USA 8852-7 (1996).
187. Mitton-Fry R M, DeGregorio S J, Wang J, Steitz T A, Steitz J A. Science 1244-7 (2010).
188. Forster A C, Symons R H. Cell. 1987.
189. Weinberg Z, Kim P B, Chen T H, Li S, Harris K A, Lunse C E, Breaker R R. Nat. Chem. Biol. 2015.
190. Feldstein P A, Buzayan J M, Bruening G. Gene 1989.
191. Saville B J, Collins R A. Cell. 1990.
192. Roth A, Weinberg Z, Chen A G, Kim P G, Ames T D, Breaker R R. Nat Chem Biol. 2013.
193. Borchardt E K, et al. RNA 1921-30 (2015).
194. Zhang Y, et al. Mol Cell 792-806 (2013).
195. Dang Y, et al. Genome Biol 280 (2015).
196. Schaefer M, Kapoor U, and Jantsch M F. Open Biol 170077 (2017).
197. Nahar S, et al. Chem Comm 2377-80 (2018).
198. Gao Y and Zhao Y. J Integr Plant Biol 343-9 (2014).
199. Dubois N, Marquet R, Paillart J, Bernacchi S. Front Microbiol 527 (2018).
200. Costa M and Michel F. EMBO J 1276-85 (1995).
201. Hu J H, et al. Nature 57-63 (2018).
202. Furukawa K, Gu H, Breaker R R. Methods Mol Biol 209-20 (2014).
203. Zettler, J., Schutz, V. & Mootz, H. D. The naturally split Npu DnaE intein exhibits an extraordinarily high rate in the protein trans-splicing reaction. FEBS Lett. 583, 909-914 (2009).
204. Kugler, S., Kilic, E. & Bahr, M. Human synapsin 1 gene promoter confers highly neuron-specific long-term transgene expression from an adenoviral vector in the adult rat brain depending on the transduced area. Gene Ther. 10, 337-347 (2003).
205. de Felipe, P., Hughes, L. E., Ryan, M. D. & Brown, J. D. Co-translational, intraribosomal cleavage of polypeptides by the foot-and-mouth disease virus 2A peptide. J. Biol. Chem. 278, 11441-11448 (2003).
206. Levy, J. M. & Nicoll, R. A. Membrane-associated guanylate kinase dynamics reveal regional and developmental specificity of synapse stability. J. Physiol. 595, 1699-1709 (2017).
207. Li, B. & Dewey, C. N. RSEM: accurate transcript quantification from RNA-Seq data with or without a reference genome. BMC Bioinformatics 12, 323 (2011).
208. Ritchie, M. E. et al. limma powers differential expression analyses for RNA-sequencing and microarray studies. Nucleic Acids Res. 43, e47-e47 (2015).

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

LENGTHY TABLES
The patent application contains a lengthy table section. A copy of the table is available in electronic form from the USPTO web site (https://seqdata.uspto.gov/?pageRequest=docDetail&DocID=US20230357766A1). An electronic copy of the table will also be available from the USPTO upon request and payment of the fee set forth in 37 CFR 1.19(b)(3).

1. A pegRNA for prime editing comprising a guide RNA and at least one nucleic acid extension arm comprising a DNA synthesis template and a primer binding site, wherein the extension arm comprises a nucleic acid moiety attached thereto selected from the group consisting of a toe-loop, hairpin, stem-loop, pseudoknot, aptamer, G-quadraplex, tRNA, riboswitch, or ribozyme.

2. The pegRNA of claim 1, wherein the nucleic acid moiety is attached to the 3′ end of the extension arm.

3. The pegRNA of claim 1, wherein the nucleic acid moiety is attached to the 5′ end of the extension arm.

4. The pegRNA of claim 1, wherein the pseudoknot is a Mpknot1 moiety having a nucleotide sequence selected from the group consisting of: SEQ ID NO: 195 (Mpknot1), SEQ ID NO: 196 (Mpknot1 3′ trimmed), SEQ ID NO: 197 (Mpknot1 with 5′ extra), SEQ ID NO: 198 (Mpknot1 U38A), SEQ ID NO: 199 (Mpknot1 U38A A29C), SEQ ID NO: 200 (MMLC A29C), SEQ ID NO: 201 (Mpknot1 with 5′ extra and U38A), SEQ ID NO: 202 (Mpknot1 with 5′ extra and U38A A29C), and SEQ ID NO: 203 (Mpknot1 with 5′ extra and A29C), or a nucleotide sequence having at least 80% sequence identity therewith.

5. The pegRNA of claim 1, wherein the G-quadruplex has a nucleotide sequence selected from the group consisting of: SEQ ID NO: 204 (tns1), SEQ ID NO: 205 (stk40), SEQ ID NO: 206 (apc2), SEQ ID NO: 207 (ceacam4), SEQ ID NO: 208 (pitpnm3), SEQ ID NO: 209 (rlf), SEQ ID NO: 210 (erc1), SEQ ID NO: 211 (ube3c), SEQ ID NO: 212(taf15), SEQ ID NO: 213 (stard3), and SEQ ID NO: 214 (g2), or a nucleotide sequence having at least 80% sequence identity therewith.

6. The pegRNA of claim 1, wherein the evopreq1 has a nucleotide sequence selected from the group consisting of: SEQ ID NO: 215 (evopreq1), SEQ ID NO: 216 (evopreq1motif1), SEQ ID NO: 217 (evopreq1motif2), SEQ ID NO: 218 (evopreq1motif3), SEQ ID NO: 219 (shorter preq1-1), SEQ ID NO: 220 (preq1-1 G5C (mut1)), and SEQ ID NO: 221 (preq1-1 G15C (mut2)), or a nucleotide sequence having at least 80% sequence identity therewith.

7. The pegRNA of claim 1, wherein the tRNA moiety has a nucleotide sequence of SEQ ID NO: 222, or a nucleotide sequence having at least 80% sequence identity therewith.

8. The pegRNA of claim 1, wherein the nucleic acid moiety has a nucleotide sequence of SEQ ID NO: 223 (xrn1), or a nucleotide sequence having at least 80% sequence identity therewith.

9. The pegRNA of claim 1, wherein the nucleic acid moiety has a nucleotide sequence of SEQ ID NO: 224 (grp1 intron P4P6), or a nucleotide sequence having at least 80% sequence identity therewith.

10. The pegRNA of any of claims 1-9, wherein the nucleic acid moiety is attached to the pegRNA by a linker.

11. The pegRNA of claim 10, wherein the linker has a nucleotide sequence selected from the group consisting of SEQ ID NOs: 225-236.

12. The pegRNA of claim 10, wherein the linker is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides in length, wherein the linker is no longer than 50 nucleotides.

13. The pegRNA of claim 10, wherein the linker is 8 nucleotides in length.

14. The pegRNA of claim 1, wherein the extension arm is positioned at the 3′ or 5′ end of the guide RNA, and wherein the nucleic acid extension arm is DNA or RNA.

15. The pegRNA of claim 1, wherein the pegRNA is capable of binding to a napDNAbp and directing the napDNAbp to a target DNA sequence.

16. The pegRNA of claim 15, wherein the target DNA sequence comprises a target strand and a complementary non-target strand.

17. The pegRNA of claim 16, wherein the guide RNA hybridizes to the target strand to form an RNA-DNA hybrid and an R-loop.

18. The pegRNA of claim 1, wherein the nucleic acid extension arm is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides.

19. The pegRNA of claim 1, wherein the DNA synthesis template is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length.

20. The pegRNA of claim 1, wherein the DNA synthesis template encodes a desired edit.

21. The pegRNA of claim 1, wherein the primer binding site is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides in length.

22. A complex for prime editing comprising:
(a) fusion protein comprising a nucleic acid programmable DNA binding protein (napDNAbp) and a domain comprising an RNA-dependent DNA polymerase activity; and
(b) a pegRNA of any one of claims 1-21.

23. The complex of claim 22, wherein the napDNAbp has a nickase activity.

24. The complex of claim 22, wherein the napDNAbp is a Cas9 protein or variant thereof.

25. The complex of claim 22, wherein the napDNAbp is a nuclease active Cas9, a nuclease inactive Cas9 (dCas9), or a Cas9 nickase (nCas9).

26. The complex of claim 22, wherein the napDNAbp is Cas9 nickase (nCas9).

27. The complex of claim 22, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (Cas Φ), and Argonaute and optionally has a nickase activity.

28. The complex of claim 22, wherein the domain comprising an RNA-dependent DNA polymerase activity is a reverse transcriptase comprising any one of the amino acid sequences of SEQ ID NOs: 32, 34, 36, 102-128, and 132.

29. The complex of claim 22, wherein the domain comprising an RNA-dependent DNA polymerase activity is a reverse transcriptase comprising an amino acid sequence having at least 80%, 85%, 90%, 95%, 98%, or 99% sequence identity with the amino acid sequence of any one of SEQ ID NOs: 32, 34, 36, 102-128, and 132.

30. The complex of claim 22, wherein the domain comprising an RNA-dependent DNA polymerase activity is a naturally-occurring reverse transcriptase from a retrovirus or a retrotransposon.

31. A nucleic acid molecule encoding the pegRNA of any one of claim 1-19.

32. An expression vector comprising the nucleic acid molecule of claim 31, wherein the nucleic acid molecule is under the control of a promoter.

33. The expression vector of claim 32, wherein the promoter is a polIII promoter.

34. The expression vector of claim 32, wherein the promoter is a U6 promoter.

35. The expression vector of claim 32, wherein the promoter is a U6, U6v4, U6v7, or U6v9 promoter, or a fragment thereof.

36. A cell comprising the pegRNA of any one of claims 1-21.

37. A cell comprising the complex of any one of claims 22-30.

38. A cell comprising the nucleic acid molecule of claim 31.

39. A cell comprising the expression vector of any one of claims 32-35.

40. A pharmaceutical composition comprising: (i) a pegRNA of any one of claims 1-21, a complex of any one of claims 22-30, a nucleic acid molecule of claim 31, an expression vector of any one of claims 32-35, or a cell of any one of claims 36-39, and (ii) a pharmaceutically acceptable excipient.

41. A kit composition comprising: (i) a pegRNA of any one of claims 1-21, a complex of any one of claims 22-30, a nucleic acid molecule of claim 31, an expression vector of any one of claims 32-35, or a cell of any one of claims 36-39, and (ii) a set of instructions for conducting prime editing.

42. A method of prime editing comprising contacting a target DNA sequence with a pegRNA of any of claims 1-21 and a prime editor comprising a napDNAbp and a domain having an RNA-dependent DNA polymerase activity, wherein the editing efficiency is increased as compared to the same method using a pegRNA not comprising the modification.

43. The method of claim 42, wherein the editing efficiency is increased by at least 1.5 fold.

44. The method of claim 42, wherein the editing efficiency is increased by at least 2 fold.

45. The method of claim 42, wherein the editing efficiency is increased by at least 3 fold.

46. The method of claim 42, wherein the napDNAbp has a nickase activity.

47. The method of claim 42, wherein the napDNAbp is a Cas9 protein or variant thereof.

48. The method of claim 47, wherein the napDNAbp is a nuclease active Cas9, a nuclease inactive Cas9 (dCas9), or a Cas9 nickase (nCas9).

49. The method of claim 48, wherein the napDNAbp is Cas9 nickase (nCas9).

50. The method of claim 42, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity.

51. The method of claim 42, wherein the domain comprising an RNA-dependent DNA polymerase activity is a reverse transcriptase comprising any one of the amino acid sequences of SEQ ID NOs: 32, 34, 36, 102-128, and 132.

52. The method of claim 42, wherein the domain comprising an RNA-dependent DNA polymerase activity is a reverse transcriptase comprising an amino acid sequence having at least 80%, 85%, 90%, 95%, 98%, or 99% sequence identity with the amino acid sequence of any one of SEQ ID NOs: 32, 34, 36, 102-128, and 132.

53. The method of claim 42, wherein the domain comprising an RNA-dependent DNA polymerase activity is a naturally-occurring reverse transcriptase from a retrovirus or a retrotransposon.

54. The pegRNA of claim 10, wherein the linker is designed by a computational method of claim 56.

55. A method for precisely installing a nucleotide edit in a double stranded target DNA sequence, the method comprising: contacting the double stranded target DNA sequence with a prime editor comprising a nucleic acid programmable DNA binding protein (napDNAbp), a DNA polymerase, and a prime editing guide RNA (PEgRNA), wherein the PEgRNA comprises:
(a) a spacer that hybridizes to a first strand of the double stranded target DNA sequence;
(b) an extension arm that hybridizes to a second strand of the double stranded target DNA sequence;
(c) a DNA synthesis template comprising the nucleotide edit;
(d) a gRNA core that interacts with the napDNAbp;
(e) a nucleic acid moiety attached thereto selected from the group consisting of a toe-loop, hairpin, stem-loop, pseudoknot, aptamer, G-quadraplex, tRNA, riboswitch, or ribozyme; and
(f) a linker that couples the nucleic acid moiety to the pegRNA,
wherein the linker is designed by a computational model; and
wherein the PEgRNA directs the prime editor to install the nucleotide edit in the double stranded target DNA sequence.

56. A method for identifying at least one nucleic acid linker for coupling a prime editing guide RNA (pegRNA) to a nucleic acid moiety, the method comprising:
using at least one computer hardware processor to perform:
generating a plurality of nucleic acid linker candidates including a first nucleic acid linker candidate;
identifying the at least one nucleic acid linker from among the plurality of nucleic acid linker candidates at least in part by:
calculating multiple scores for each of at least some of the plurality of nucleic acid linker candidates, the calculating comprising calculating a first set of scores for the first nucleic acid linker candidate, the first set of scores comprising:
a first score indicative of a degree of interaction between the first nucleic acid linker candidate and a first region of the pegRNA;
a second score indicative of a degree of interaction between the first nucleic acid linker candidate and a second region of the pegRNA; and
identifying the at least one nucleic acid linker from among the at least some of the plurality of nucleic acid linker candidates using the calculated multiple scores; and
outputting information indicative of the at least one nucleic acid linker.

57. The method of claim 56, wherein the first score is indicative of a degree to which the first nucleic acid linker candidate is predicted to avoid interaction with the first region of the pegRNA, and wherein the second score is indicative of a degree to which the first nucleic acid linker candidate is predicted to avoid interaction with the second region of the pegRNA.

58. The method of claim 57, wherein the first region comprises a primer binding site (PBS) of the pegRNA.

59. The method of claim 58, wherein the second region comprises a spacer of the pegRNA.

60. The method of claim 57, wherein the first set of scores further comprises a third score indicative of a degree to which the first nucleic acid linker candidate is predicted to avoid interaction with a third region of the pegRNA and a fourth score indicative of a degree to which the first nucleic acid linker candidate is predicted to avoid interaction with a fourth region of the pegRNA.

61. The method of claim 60, wherein the third region comprises a DNA synthesis template.

62. The method of claim 61, wherein the fourth region comprises a gRNA core that interacts with a nucleic acid programmable DNA binding protein (napDNAbp).

63. The method of claim 60,
wherein the pegRNA is for installing a nucleotide edit in a double stranded target DNA sequence,
wherein the pegRNA comprises:
a spacer that hybridizes to a first strand of the double stranded target DNA sequence,
an extension arm that hybridizes to a second strand of the double stranded target DNA sequence, the extension arm comprising a primer binding site (PBS) and a DNA synthesis template comprising the nucleotide edit, and
a gRNA core that interacts with a nucleic acid programmable DNA binding protein napDNAbp, and
wherein the first region comprises the PBS, the second region comprises the spacer, the third region comprises the DNA synthesis template, and the fourth region comprises the gRNA core.

64. The method of claim 56, wherein the plurality of nucleic acid linker candidates comprises a second nucleic acid linker candidate, and wherein identifying the at least one nucleic acid linker from among the at least some of the plurality of nucleic acid linker candidates using the calculated multiple scores comprises:
comparing the first set of scores for the first nucleic acid linker candidate with a second set of scores for the second nucleic acid linker candidate.

65. The method of claim 64, wherein:
the first region comprises a primer binding site (PBS),
the first score in the first set of scores is indicative of a degree to which the first nucleic acid linker candidate is predicted to avoid interaction with the first region of the pegRNA,
a third score in the second set of scores is indicative of a degree to which the second nucleic acid linker candidate is predicted to avoid interaction with the first region of the pegRNA, and
comparing the first set of scores with the second set of scores comprises:
comparing the first score with the third score.

66. The method of claim 65, wherein when the first score is equal to or is within a threshold distance of the third score, comparing the first set of scores with the second set of scores further comprises:
comparing a score, other than the first score, in the first set of scores with another score, other than the third score, in the second set of scores.

67. A PEgRNA for prime editing comprising (i) a guide RNA comprising a spacer and (ii) at least one nucleic acid extension arm comprising a DNA synthesis template, a primer binding site, a toehold motif, and an additional nucleic acid moiety.

68. The PEgRNA of claim 67, wherein the toehold motif and the additional nucleic acid moiety are attached to the 3′ end of the extension arm.

69. The PEgRNA of claim 67 or 68, wherein the toehold motif is attached to the 3′ end of the extension arm, and the additional nucleic acid moiety is attached to the 3′ end of the toehold motif.

70. The PEgRNA of any one of claims 67-69, wherein the toehold motif is attached to the PEgRNA by a linker.

71. The PEgRNA of claim 70, wherein the linker is at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length.

72. The PEgRNA of any one of claims 67-71, wherein the PEgRNA is capable of binding to a nucleic acid programmable DNA binding protein (napDNAbp) of a prime editor and directing the napDNAbp to a target DNA sequence.

73. A prime editing system for site specific genome modification comprising (a) a PEgRNA of any one of claims 67-72, and (b) a prime editor comprising (i) a napDNAbp, (ii) a DNA polymerase, and (iii) a portion that binds to the toehold motif of the PEgRNA.

74. The system of claim 73, wherein the portion of the prime editor that binds to the toehold motif of the PEgRNA is fused to the N-terminal end of the prime editor.

75. The system of claim 73, wherein the portion of the prime editor that binds to the toehold motif of the PEgRNA is fused to the C-terminal end of the prime editor.

76. The system of any one of claims 73-75, wherein the portion of the prime editor that binds to the toehold motif of the PEgRNA is fused to the prime editor by a linker.

77. The system of claim 76, wherein the linker is at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more than 30 amino acids in length.

78. The system of claim 76 or 77, wherein the linker comprises an xten linker.

79. The system of any one of claims 73-78, wherein the portion of the prime editor that binds to the toehold motif of the PEgRNA comprises an MS2 bacteriophage coat protein.

80. The system of any one of claims 73-79, wherein the napDNAbp has a nickase activity.

81. The system of any one of claims 73-80, wherein the napDNAbp is a Cas9 protein or a variant thereof.

82. The system of any one of claims 73-79, wherein the napDNAbp is a nuclease active Cas9, a nuclease inactive Cas9 (dCas9), or a Cas9 nickase (nCas9).

83. The system of any one of claims 73-79, wherein the napDNAbp is a Cas9 nickase (nCas9).

84. The system of any one of claims 73-79, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity.

85. A polynucleotide comprising the PEgRNA of any one of claims 67-72.

86. A vector comprising the polynucleotide of claim 85.

87. A cell comprising the PEgRNA of any one of claims 67-72, the system of any one of claims 73-84, the polynucleotide of claim 85, or the vector of claim 86.

88. A pharmaceutical composition comprising (i) the PEgRNA of any one of claims 67-72, the system of any one of claims 73-84, the polynucleotide of claim 85, or the vector of claim 86, and (ii) a pharmaceutically acceptable excipient.

89. A kit comprising the PEgRNA of any one of claims 67-72, the system of any one of claims 73-84, the polynucleotide of claim 85, the vector of claim 86, or the cell of claim 87.

90. A method of prime editing comprising providing a target DNA sequence to the system of any one of claims 73-84, wherein the target DNA sequence is contacted with the PEgRNA and the prime editor of the system.

91. A pair of PEgRNAs for prime editing comprising
(i) a first PEgRNA comprising a guide RNA and at least one nucleic acid extension arm comprising a DNA synthesis template and a primer binding site, wherein the extension arm comprises a nucleic acid moiety attached thereto selected from the group consisting of a toe-loop, hairpin, stem-loop, pseudoknot, aptamer, G-quadraplex, tRNA, riboswitch, or ribozyme; and
(ii) a second PEgRNA comprising a second strand nicking guide RNA, wherein the second strand nicking guide RNA comprises at least one nucleic acid extension arm comprising a DNA synthesis template and a primer binding site.

92. The pair of PEgRNAs of claim 91, wherein the first PEgRNA and the second PEgRNA are each capable of binding to a nucleic acid programmable DNA binding protein (napDNAbp) of a prime editor and directing the napDNAbp to a target DNA sequence.

93. A prime editing system for site specific genome modification comprising (a) a pair of PEgRNAs of claim 91 or 92, and (b) at least one prime editor comprising a napDNAbp and a DNA polymerase.

94. The system of claim 93, wherein the system comprises a first prime editor and second prime editor, each comprising a napDNAbp and a DNA polymerase.

95. The system of claim 94, wherein the napDNAbp of the first prime editor binds to the first PEgRNA of the pair of PEgRNAs, and wherein the napDNAbp of the second prime editor binds to the second PEgRNA of the pair of PEgRNAs.

96. The system of any one of claims 93-95, wherein the napDNAbp has a nickase activity.

97. The system of any one of claims 93-95, wherein the napDNAbp is a Cas9 protein or a variant thereof.

98. The system of any one of claims 93-95, wherein the napDNAbp is a nuclease active Cas9, a nuclease inactive Cas9 (dCas9), or a Cas9 nickase (nCas9).

99. The system of any one of claims 93-95, wherein the napDNAbp is a Cas9 nickase (nCas9).

100. The system of any one of claims 93-95, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity.

101. A polynucleotide comprising the PEgRNA of claim 91 or 92.

102. A vector comprising the polynucleotide of claim 101.

103. A cell comprising the PEgRNA of claim 91 or 92, the system of any one of claims 93-100, the polynucleotide of claim 101, or the vector of claim 102.

104. A pharmaceutical composition comprising (i) the PEgRNA of claim 91 or 92, the system of any one of claims 93-100, the polynucleotide of claim 101, or the vector of claim 102, and (ii) a pharmaceutically acceptable excipient.

105. A kit comprising the PEgRNA of claim 91 or 92, the system of any one of claims 93-100, the polynucleotide of claim 101, the vector of claim 102, or the cell of claim 103.

106. A method of prime editing comprising providing a target DNA sequence to the system of any one of claims 93-100, wherein the target DNA sequence is contacted with the pair of PEgRNAs and the one or more prime editors of the system.

107. A PEgRNA comprising (i) a guide RNA comprising a spacer and (ii) at least one nucleic acid extension arm comprising a DNA synthesis template and a primer binding site, wherein the primer binding site comprises one or more modified nucleotides which result in a greater reduction in binding affinity of the primer binding site to the spacer than of the primer binding site to a protospacer sequence on a target DNA molecule.

108. The PEgRNA of claim 107, wherein the one or more modified nucleotides comprise genetic mutations.

109. The PEgRNA of claim 107, wherein the one or more modified nucleotides comprise chemically-modified nucleotides.

110. A prime editing system for site specific genome modification comprising (a) a pair of PEgRNAs of any one of claims 107-109, and (b) at least one prime editor comprising a napDNAbp and a DNA polymerase.

111. The system of claim 110, wherein the system comprises a first prime editor and second prime editor, each comprising a napDNAbp and a DNA polymerase.

112. The system of claim 111, wherein the napDNAbp of the first prime editor binds to the first PEgRNA of the pair of PEgRNAs, and wherein the napDNAbp of the second prime editor binds to the second PEgRNA of the pair of PEgRNAs.

113. The system of any one of claims 110-112, wherein the napDNAbp has a nickase activity.

114. The system of any one of claims 110-112, wherein the napDNAbp is a Cas9 protein or a variant thereof.

115. The system of any one of claims 110-112, wherein the napDNAbp is a nuclease active Cas9, a nuclease inactive Cas9 (dCas9), or a Cas9 nickase (nCas9).

116. The system of any one of claims 110-112, wherein the napDNAbp is a Cas9 nickase (nCas9).

117. The system of any one of claims 110-112, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity.

118. A polynucleotide comprising the PEgRNA of any one of claims 107-109.

119. A vector comprising the polynucleotide of claim 118.

120. A cell comprising the PEgRNA of any one of claims 107-109, the system of any one of claims 110-117, the polynucleotide of claim 118, or the vector of claim 119.

121. A pharmaceutical composition comprising (i) the PEgRNA of any one of claims 107-109, the system of any one of claims 110-117, the polynucleotide of claim 118, or the vector of claim 119, and (ii) a pharmaceutically acceptable excipient.

122. A kit comprising the PEgRNA of any one of claims 107-109, the system of any one of claims 110-117, the polynucleotide of claim 118, the vector of claim 119, or the cell of claim 120.

123. A method of prime editing comprising providing a target DNA sequence to the system of any one of claims 110-117, wherein the target DNA sequence is contacted with the pair of PEgRNAs and the one or more prime editors of the system.

124. A method of correcting one or more mutations in a CDKL5 gene by prime editing using a single pegRNA comprising contacting a target DNA sequence with a prime editor comprising (i) a napDNAbp and (ii) a domain having an RNA-dependent DNA polymerase activity, and a pegRNA, wherein the pegRNA targets the prime editor to a CDKL5 gene comprising one or more mutations.

125. The method of claim 124, wherein the pegRNA is provided in FIG. 146.

126. The method of claim 124, wherein the pegRNA is provided in FIG. 148.

127. The method of claim 124, wherein the mutation in the CDKL5 gene comprises a 1412delA mutation.

128. The method of claim 124, wherein the one or more mutations encodes a V1721, A173D, R175S, W176G, W176R, Y177C, R178P, P180L, E181A, or L182P substitution.

129. The method of claim 124, wherein the napDNAbp has a nickase activity.

130. The method of claim 124, wherein the napDNAbp is a Cas9 protein or variant thereof.

131. The method of claim 124, wherein the napDNAbp is a nuclease active Cas9, a nuclease inactive Cas9 (dCas9), or a Cas9 nickase (nCas9).

132. The method of claim 124, wherein the napDNAbp is Cas9 nickase (nCas9).

133. The method of claim 124, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity.

134. The method of claim 124, wherein the domain comprising an RNA-dependent DNA polymerase activity is a reverse transcriptase.

135. A method of treating a plurality of subjects having CDKL5 deficiency disorder caused by different mutations in the CDKL5 gene comprising contacting a target DNA sequence with a prime editor comprising (i) a napDNAbp and (ii) a domain having an RNA-dependent DNA polymerase activity, and a singular pegRNA, wherein the singular pegRNA is capable of targeting the prime editor to the CDKL5 gene in any of the plurality of subjects to result in a repaired CDKL5 gene in a mutation-agnostic manner.

136. The method of claim 135, wherein the pegRNA is provided in FIG. 148.

137. The method of claim 135, wherein the pegRNA is provided in FIG. 150.

138. The method of claim 135, wherein the mutation in the CDKL5 gene comprises a 1412delA mutation.

139. The method of claim 135, wherein the one or more mutations encodes a V1721, A173D, R175S, W176G, W176R, Y177C, R178P, P180L, E181A, or L182P substitution.

140. The method of claim 135, wherein the napDNAbp has a nickase activity.

141. The method of claim 135, wherein the napDNAbp is a Cas9 protein or variant thereof.

142. The method of claim 135, wherein the napDNAbp is a nuclease active Cas9, a nuclease inactive Cas9 (dCas9), or a Cas9 nickase (nCas9).

143. The method of claim 135, wherein the napDNAbp is Cas9 nickase (nCas9).

144. The method of claim 135, wherein the napDNAbp is selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute, and optionally has a nickase activity.

145. The method of claim 135, wherein the domain comprising an RNA-dependent DNA polymerase activity is a reverse transcriptase.
